(19) United States
(12) Patent Application Publication
(10) Pub. No.: US 2007/0037807 A1
(43) Pub. Date: Feb. 15, 2007

(54) Pyridine compounds as inhibitors of dipeptidyl peptidase IV

(76) Inventors: Satoru Oi, Osaka (JP); Hironobu Maezaki, Osaka (JP); Nobuhiro Suzuki, Ibaraki (JP)

Correspondence Address: Mark Chao;Takeda Pharmaceuticals North America Inc, Suite 500, 475 Half Day Road, Lincolnshire, IL, 60069, US

(21) Appl. No.: 10/577,561

(22) PCT Filed: Oct. 29, 2004
(86) PCT No.: PCT/JP04/16457

§ 371 Date: Apr. 28, 2006

(30) Foreign Application Priority Data
Oct. 31, 2003 (JP) 2003-373776
Feb. 6, 2004 (JP) 2004-030491
Jun. 3, 2004 (JP) 2004-165977

(51) Int. Cl. A61K 31/5377; A61K 31/4439; A61K 31/44; C07D 413/02

(52) U.S. Cl. 514/235.2; 514/352; 514/340; 514/341; 514/355; 544/128; 546/272.1; 546/310

(57) ABSTRACT

A compound represented by the formula 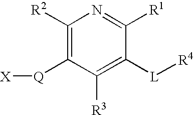 wherein
R¹ and R² are the same or different and each is an optionally substituted hydrocarbon group or an optionally substituted hydroxy group; R³ is an optionally substituted aromatic group; R⁴ is an optionally substituted amino group; L is a divalent chain hydrocarbon group; Q is a bond or a divalent chain hydrocarbon group; and X is a hydrogen atom, a cyano group, a nitro group, an acyl group, a substituted hydroxy group, an optionally substituted thiol group, an optionally substituted amino group or an optionally substituted cyclic group; provided that when X is an ethoxycarbonyl group, then Q is a divalent chain hydrocarbon group. The compound has a peptidase inhibitory action, is useful as an agent for the prophylaxis or treatment of diabetes and the like, and is superior in efficacy, duration of action, specificity, lower toxicity and the like.

### TECHNICAL FIELD

The present invention relates to a pyridine compound having a peptidase inhibitory activity, which is useful as an agent for the prophylaxis or treatment of diabetes and the like.

### BACKGROUND ART

Peptidase is known to relate to various diseases. Dipeptidyl dipeptidase-IV (hereinafter sometimes to be abbreviated as DPP-IV), which is one kind of peptidases, is serine protease that specifically binds with a peptide containing proline (or alanine) at the 2nd from the N-terminal and cleaves the C-terminal side of the proline (or alanine) to produce dipeptide. DPP-IV has been shown to be the same molecule as CD26, and reported to be also involved in the immune system. While the role of DPP-IV in mammals has not been entirely clarified, it is considered to play an important role in the metabolism of neuropeptides, activation of T cells, adhesion of cancerous cells to endothelial cells, invasion of HIV into cells and the like. Particularly, from the aspect of glycometabolism, DPP-IV is involved in the inactivation of GLP-1 (glucagon-like peptide-1) and GIP (Gastric inhibitory peptide/Glucose-dependent insulinotropic peptide), which are incretins. With regard to GLP-1, moreover, it is known that the physiological activity of GLP-1 is markedly impaired because it has a short plasma half-life of 1-2 minutes, and GLP-1(9-36)amide, which is a degradation product by DPP-IV, acts on GLP-1 receptor as an antagonist, thus decomposing GLP-1 by DPP-IV. It is also known that suppression of degradation of GLP-1 by inhibiting DPP-IV activity leads to potentiation of physiological activity that GLP-1 shows, such as glucose concentration-dependent insulin secretagogue effect and the like. From these facts, a compound having a DPP-IV inhibitory activity is expected to show effect on impaired glucose tolerance, postprandial hyperglycemia and fasting hyperglycemia observed in type I and type II diabetes and the like, obesity or diabetic complications associated therewith and the like.

As pyridine compound, the following compounds have been reported.
(1) A compound represented by the formula 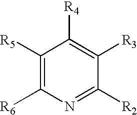
   wherein R₂ and R₆ are each independently hydrogen, hydroxy, alkyl and the like; R₃ is hydroxy, amido and the like; R₄ is hydrogen, hydroxy, halogen and the like; and R₅ is hydrogen, hydroxy, halogen and the like, which has a cholesterol·ester·transfer·protein (hereinafter to be abbreviated as CETP) inhibitory action (see WO99/41237).
(2) A compound represented by the formula 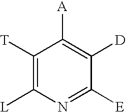
   wherein A is C₆₋₁₀ aryl optionally substituted by halogen and the like; D is straigh-chain or branched alkyl having 8 or less carbon atoms optionally substituted by hydroxy; E and L are the same or different and each is straigh-chain or branched alkyl having 8 or less carbon atoms optionally substituted by C₃₋₈ cycloalkyl, and the like; T is R⁷—X— or R⁸—(R⁹)(R¹⁰)C— (wherein R⁷and R⁸ are the same or different and each is C₃₋₈ cycloalkyl, C₆₋₁₀ aryl and the like; R⁹ is hydrogen and the like; R¹⁰ is hydrogen, halogen, azido and the like), which has a CETP inhibitory action or a glucagon antagonistic action; a compound represented by the formula 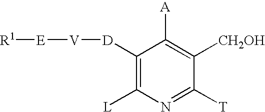
   wherein A is C₆₋₁₀ aryl optionally substituted by halogen and the like; D and E are the same or different and each is straigh-chain or branched alkyl having 8 or less carbon atoms optionally substituted by hydroxy; V is O, S or NR⁵ (wherein R⁵ is hydrogen, straigh-chain or branched alkyl having 6 or less carbon atoms, or phenyl); R¹ is C₃₋₆ cycloalkyl, C₆₋₁₀ aryl and the like; L and T are the same or different and each is trifluoromethyl and the like; and a compound represented by the formula 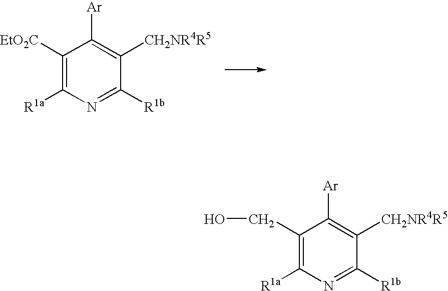
   wherein Ar is optionally substituted aromatic or heteroaromatic group; R⁴ and R⁵ are independently hydrogen, C₁₋₆ alkyl and the like; R^{1a} and R^{1b} are independently trifluoromethyl, C₁₋₆ alkyl and the like (see WO98/04528, U.S. Pat. No. 6,218,431).
(3) A compound represented by the formula 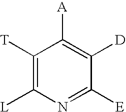
   wherein A and E are the same or different and each is C₆₋₁₀ aryl optionally substituted by halogen and the like; D is straigh-chain or branched alkyl having 8 or less carbon atoms optionally substituted by hydroxy; L is C₃₋₈ cycloalkyl, straigh-chain or branched alkyl having 8 or less carbon atoms, and the like; T is R³—X— or R⁴—(R⁵)(R⁶)C— (wherein R³ and R⁴ are the same or different and each is C₃₋₈ cycloalkyl, C₆₋₁₀ aryl and the like; R⁵ is hydrogen and the like; R⁶ is hydrogen, halogen, azido and the like), or a salt thereof, having a CETP inhibitory action (see U.S. Pat. No. 5,925,645).
(4) A compound represented by the formula 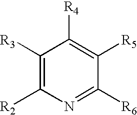
   wherein R₂ and R₆ are independent bromoalkyl, chloroalkyl and the like; R₄ is alkyl, cycloalkylalkyl, alkylthioalkyl, cycloalkyl, alkoxyalkyl or dialkylaminoalkyl; the one of R₃ and R₅ is CO—Y (wherein Y is alkylthio, alkoxy or N-containing heterocyclic group), the other is —(—C(R⁹)(R¹⁰)-)n-X (wherein n is an integer of 1-3; R⁹ and R¹⁰ are independently hydrogen, alkyl and the like; X is halogen, OH and the like) and the like, or a salt thereof, which has a herbicide action (see WO92/20659).
(5) A compound represented by the formula 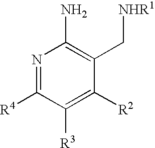
   wherein R¹ is hydrogen or lower alkyl; R² is heterocyclic group or aryl each optionally substituted by lower alkyl and the like; R³ and R⁴ may form a phenyl ring and the like each optionally substituted by halogen and the like, together with the carbon atoms bonded thereto, or a salt thereof, which has a DPP-IV inhibitory action (see WO03/068748).
(6) A compound represented by the formula 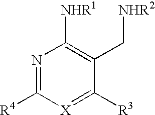
   wherein X is N or CR⁵ (wherein R⁵ is hydrogen or lower alkyl); R¹ and R² are independently hydrogen or lower alkyl; R³ is heterocyclic group or aryl each optionally substituted by lower alkyl and the like; R⁴ is lower alkyl and the like, or a salt thereof, which has a DPP-IV inhibitory action (see WO03/068757).

However, there is no report on the compound of the present invention.

### DISCLOSURE OF THE INVENTION

There is a demand for the development of a compound having a peptidase inhibitory action, which is useful as an agent for the prophylaxis or treatment of diabetes and the like and superior in efficacy, duration of action, specificity, lower toxicity and the like.

The present inventors have first found that a compound represented by the formula 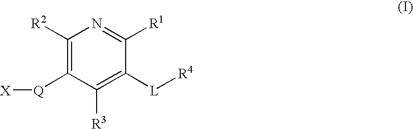
wherein
R¹ and R² are the same or different and each is an optionally substituted hydrocarbon group or an optionally substituted hydroxy group;
R³ is an optionally substituted aromatic group;
R⁴ is an optionally substituted amino group;
L is a divalent chain hydrocarbon group;
Q is a bond or a divalent chain hydrocarbon group; and
X is a hydrogen atom, a cyano group, a nitro group, an acyl group, a substituted hydroxy group, an optionally substituted thiol group, an optionally substituted amino group or an optionally substituted cyclic group; provided that
when X is an ethoxycarbonyl group, then Q is a divalent chain hydrocarbon group, and that the compound is not 2,6-diisopropyl-3-methylaminomethyl-4-(4-fluorophenyl)-5-pentylpyridine;
2,6-diisopropyl-3-aminomethyl-4-(4-fluorophenyl)-5-pentylpyridine;
2,6-diisopropyl-3-(dimethylamino)methyl-4-(4-fluorophenyl)-5-pentylpyridine;
2,6-diisopropyl-3-(ethylamino)methyl-4-(4-fluorophenyl)-5-pentylpyridine; and
3-(tert-butyldimethylsilyloxymethyl)-2,6-diisopropyl-4-(4-fluorophenyl)-5-(indolyl-5-aminomethyl)pyridine, or a salt thereof
   [hereinafter sometimes to be abbreviated as compound (I)], which is characterized by a chemical structure wherein an optionally substituted amino group is bonded to the 3-position of pyridine ring via a divalent chain hydrocarbon group and an optionally substituted aromatic group is bonded to the 4-position, has a superior peptidase inhibitory action and is useful as an agent for the prophylaxis or treatment of diabetes and the like. Based on this finding, the present inventors have conducted intensive studies and completed the present invention.

Accordingly, the present invention relates to
1) compound (I);
2) compound (I), wherein R¹ and R² are the same or different and each is an optionally substituted hydrocarbon group, and X is a cyano group, a nitro group, an acyl group, a substituted hydroxy group, an optionally substituted thiol group or an optionally substituted cyclic group;
3) compound (I), wherein the acyl group for X is a carboxyl group;
4) compound (I), wherein R¹ and R² are the same or different and each is a C₁₋₁₀ alkyl group optionally substituted by 1 to 3 substituent(s) selected from a C₃₋₁₀ cycloalkyl group, a C₁₋₆ alkoxy-carbonyl group and a C₁₋₆ alkoxy group;
5) compound (I), wherein R³ is a C₆₋₁₄ aryl group optionally substituted by 1 to 3 substituent(s) selected from a C₁₋₆ alkyl group optionally substituted by 1 to 3 halogen atom(s) and a halogen atom;
6) compound (I), wherein R⁴ is an amino group;
7) compound (I), wherein L is a C₁₋₁₀ alkylene group;
8) compound (I), wherein Q is a bond;
9) compound (I), wherein X is an acyl group, a substituted hydroxy group, an optionally substituted thiol group or an optionally substituted amino group;
10) compound (I), wherein X is a carboxyl group;
11) compound (I), which is 5-(aminomethyl)-2-methyl-4-(4-methylphenyl)-6-neopentylnicotinic acid;
5-(aminomethyl)-6-isobutyl-2-methyl-4-(4-methylphenyl)nicotinic acid;
methyl 3-{[5-(aminomethyl)-6-isobutyl-2-methyl-4-(4-methylphenyl)pyridin-3-yl]methoxy}-1-methyl-1H-pyrazole-4-carboxylate;
{[2-isobutyl-6-methyl-4-(4-methylphenyl)-5-(2-morpholin-4-yl-2-oxoethyl)pyridin-3-yl]methyl}amine;
methyl 3-({[5-(aminomethyl)-6-isobutyl-2-methyl-4-(4-methylphenyl)pyridin-3-yl]acetyl}amino)benzoate;
N-[5-(aminomethyl)-6-isobutyl-2-methyl-4-(4-methylphenyl)pyridin-3-yl]isoxazole-4-carboxamide, or a salt thereof;
12) a prodrug of compound (I);
13) a pharmaceutical agent comprising compound (I) or a prodrug thereof;
14) the pharmaceutical agent of 13) above, which is an agent for the prophylaxis or treatment of diabetes, diabetic complications, impaired glucose tolerance or obesity;
15) a peptidase inhibitor comprising compound (I) or a prodrug thereof;
16) the inhibitor of 15) above, wherein the peptidase is dipeptidyl dipeptidase-IV;
17) use of compound (I) or a prodrug thereof for the production of an agent for the prophylaxis or treatment of diabetes, diabetic complications, impaired glucose tolerance or obesity;
18) use of compound (I) or a prodrug thereof for the production of a peptidase inhibitor;
19) a method for the prophylaxis or treatment of diabetes, diabetic complications, impaired glucose tolerance or obesity in a mammal, which comprises administering compound (I) or a prodrug thereof to the mammal;
20) a method of inhibiting peptidase in a mammal, which comprises administering compound (I) or a prodrug thereof to the mammal;
21) a production method of a compound represented by the formula 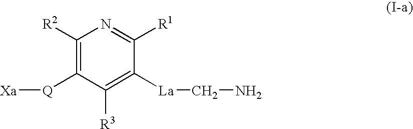
   wherein
   R¹, R², R³ and Q are as defined in compound (I);
   La is a bond or a divalent chain hydrocarbon group; and
   Xa is a hydrogen atom, a nitro group, an acyl group, a substituted hydroxy group, an optionally substituted thiol group, an optionally substituted amino group or an optionally substituted cyclic group;
      or a salt thereof, which comprises subjecting a compound represented by the formula 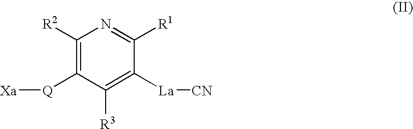
      wherein each symbol is as defined above, or a salt thereof to a reduction reaction; and the like.

The compound of the present invention has a superior peptidase inhibitory action and is useful as an agent for the prophylaxis or treatment of diabetes and the like.

### BEST MODE FOR CARRYING OUT THE INVENTION

Each symbol in the formula (I) is described in detail in the following.

As the “hydrocarbon group” of the “optionally substituted hydrocarbon group” for R¹ or R², for example, a C₄₋₁₀ alkyl group, a C₂₋₁₀ alkenyl group, a C₂₋₁₀ alkynyl group, a C₃₋₁₀ cycloalkyl group, a C₃₋₁₀ cycloalkenyl group, a C₄₋₁₀ cycloalkadienyl group, a C₆₋₁₄ aryl group, a C₇₋₁₃ aralkyl group, a C₈₋₁₃ arylalkenyl group, a C₃₋₁₀ cycloalkyl-C₁₋₆ alkyl group and the like can be mentioned.

As the C₁₋₁₀ alkyl group here, for example, methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl, heptyl, octyl, nonyl, decyl and the like can be mentioned.

As the C₂₋₁₀ alkenyl group, for example, ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl, 5-hexenyl, 1-heptenyl, 1-octenyl and the like can be mentioned.

As the C₂₋₁₀ alkynyl group, for example, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 1-heptynyl, 1-octynyl and the like can be mentioned.

As the C₃₋₁₀ cycloalkyl group, for example, cyclopropyl, cyclobutyl, cyclopentyl, cyclohexyl, cycloheptyl, cyclooctyl, bicyclo[2.2.1]heptyl, bicyclo[2.2.2]octyl, bicyclo[3.2.1]octyl, bicyclo[3.2.2]nonyl, bycyclo[3.3.1]nonyl, bicyclo[4.2.1]nonyl, bicyclo[4.3.1]decyl and the like can be mentioned.

As the C₃₋₁₀ cycloalkenyl group, for example, 2-cyclopenten-1-yl, 3-cyclopenten-1-yl, 2-cyclohexen-1-yl, 3-cyclohexen-1-yl and the like can be mentioned.

As the C₄₋₁₀ cycloalkadienyl group, for example, 2,4-cyclopentadien-1-yl, 2,4-cyclohexadien-1-yl, 2,5-cyclohexadien-1-yl and the like can be mentioned.

As the C₆₋₁₄ aryl group, for example, phenyl, naphthyl, anthryl, phenanthryl, acenaphthylenyl, biphenylyl and the like can be mentioned. Of these, phenyl, 1-naphthyl, 2-naphthyl and the like are preferable.

As the C₇₋₁₃ aralkyl group, for example, benzyl, phenethyl, naphthylmethyl, biphenylylmethyl and the like can be mentioned.

As the C₈₋₁₃ arylalkenyl group, for example, styryl and the like can be mentioned.

As the C₃₋₁₀ cycloalkyl-C₁₋₆ alkyl group, for example, cyclohexylmethyl and the like can be mentioned.

The aforementioned C₁₋₁₀ alkyl group, C₂₋₁₀ alkenyl group and C₂₋₁₀ alkynyl group optionally have 1 to 3 substituent(s) at substitutable position(s).

As these substituents, for example,
(1) a C₃₋₁₀ cycloalkyl group (e.g., cyclopropyl, cyclohexyl);
(2) a C₆₋₁₄ aryl group (e.g., phenyl, naphthyl);
(3) an aromatic heterocyclic group (e.g., thienyl, furyl, pyridyl, oxazolyl, thiazolyl, tetrazolyl, oxadiazolyl, pyrazinyl, quinolyl, indolyl) optionally substituted by 1 to 3 substituent(s) selected from a carboxyl group, a carbamoyl group, a thiocarbamoyl group and a C₁₋₆ alkoxy-carbonyl group (e.g., methoxycarbonyl, ethoxycarbonyl, propoxycarbonyl, tert-butoxycarbonyl);
(4) a non-aromatic heterocyclic group (e.g., tetrahydrofuryl, morpholino, thiomorpholino, piperidino, pyrrolidinyl, piperazinyl, oxodioxolyl, oxodioxolanyl, oxo-2-benzofuranyl, oxooxadiazolyl) optionally substituted by a C₁₋₆ alkyl group (e.g., methyl, ethyl);
(5) an amino group optionally mono- or di-substituted by substituent(s) selected from a C₁₋₆ alkyl group (e.g., methyl, ethyl), a C₁₋₆ alkyl-carbonyl group (e.g., acetyl, isobutanoyl, isopentanoyl) and a C₁₋₆ alkoxy-carbonyl group (e.g., methoxycarbonyl, ethoxycarbonyl, propoxycarbonyl, tert-butoxycarbonyl);
(6) a C₁₋₆ alkylsulfonylamino group (e.g., methylsulfonylamino);
(7) an amidino group;
(8) a C₁₋₆ alkyl-carbonyl group (e.g., acetyl, isobutanoyl, isopentanoyl);
(9) a C₁₋₆ alkoxy-carbonyl group (e.g., methoxycarbonyl, ethoxycarbonyl, propoxycarbonyl, tert-butoxycarbonyl);
(10) a C₁₋₆ alkylsulfonyl group (e.g., methylsulfonyl);
(11) a carbamoyl group optionally mono- or di-substituted by a C₁₋₆ alkyl group (e.g., methyl, ethyl) optionally substituted by 1 to 3 halogen atom(s) (e.g., fluorine, chlorine, bromine, iodine);
(12) a thiocarbamoyl group optionally mono- or di-substituted by a C₁₋₆ alkyl group (e.g., methyl, ethyl) optionally substituted by 1 to 3 halogen atom(s) (e.g., fluorine, chlorine, bromine, iodine);
(13) a sulfamoyl group optionally mono- or di-substituted by a C₁₋₆ alkyl group (e.g., methyl, ethyl) optionally substituted by 1 to 3 halogen atom(s) (e.g., fluorine, chlorine, bromine, iodine);
(14) a carboxyl group;
(15) a hydroxy group;
(16) a C₁₋₆ alkoxy group (e.g., methoxy, ethoxy) optionally substituted by 1 to 3 halogen atom(s) (e.g., fluorine, chlorine, bromine, iodine);
(17) a C₂₋₆ alkenyloxy group (e.g., ethenyloxy) optionally substituted by 1 to 3 halogen atom(s) (e.g., fluorine, chlorine, bromine, iodine);
(18) a C₃₋₁₀ cycloalkyloxy group (e.g., cyclohexyloxy);
(19) a C₇₋₁₃ aralkyloxy group (e.g., benzyloxy);
(20) a C₆₋₁₄ aryloxy group (e.g., phenyloxy, naphthyloxy);
(21) a C₁₋₆ alkyl-carbonyloxy group (e.g., acetyloxy, tert-butylcarbonyloxy);
(22) a thiol group;
(23) a C₁₋₆ alkylthio group (e.g., methylthio, ethylthio) optionally substituted by 1 to 3 halogen atom(s) (e.g., fluorine, chlorine, bromine, iodine);
(24) a C₇₋₁₃ aralkylthio group (e.g., benzylthio);
(25) a C₆₋₁₄ arylthio group (e.g., phenylthio, naphthylthio);
(26) a sulfo group;
(27) a cyano group;
(28) a azido group;
(29) a nitro group;
(30) a nitroso group;
(31) a halogen atom (e.g., fluorine, chlorine, bromine, iodine);
(32) a C₁₋₆ alkylsulfinyl group (e.g., methylsulfinyl); and the like can be mentioned.

The C₃₋₁₀ cycloalkyl group, C₃₋₁₀ cycloalkenyl group, C₄₋₁₀ cycloalkadienyl group, C₆₋₁₄ aryl group, C₇₋₁₃ aralkyl group, C₈₋₁₃ arylalkenyl group and C₃₋₁₀ cycloalkyl-C₁₋₆ alkyl group, which are exemplarily recited for the aforementioned “hydrocarbon group”, optionally have 1 to 3 substituent(s) at substitutable position(s).

As these substituents, for example, those exemplarily recited for the substituents for the aforementioned C₁₋₁₀ alkyl group and the like;
a C₁₋₆ alkyl group (e.g., methyl, ethyl) optionally substituted by 1 to 3 substituent(s) selected from a halogen atom (e.g., fluorine, chlorine, bromine, iodine), a carboxyl group, a C₁₋₆ alkoxy-carbonyl group (e.g., methoxycarbonyl, ethoxycarbonyl) and a carbamoyl group;
a C₂₋₆ alkenyl group (e.g., ethenyl, 1-propenyl) optionally substituted by 1 to 3 substituent(s) selected from a halogen atom (e.g., fluorine, chlorine, bromine, iodine), a carboxyl group, a C₁₋₆ alkoxy-carbonyl group (e.g., methoxycarbonyl, ethoxycarbonyl) and a carbamoyl group;
a C₇₋₁₃ aralkyl group (e.g., benzyl); and the like can be mentioned.

The “hydrocarbon group” of the “optionally substituted hydrocarbon group” for R¹ or R² is preferably a C₁₋₁₀ alkyl group, a C₆₋₁₄ aryl group or a C₇₋₁₃ aralkyl group, more preferably a C₁₋₁₀ alkyl group.

The “optionally substituted hydrocarbon group” for R¹ or R² is preferably
(1) a C₁₋₁₀ alkyl group optionally substituted by 1 to 3 substituent(s) selected from a C₃₋₁₀ cycloalkyl group, a C₁₋₆ alkoxy-carbonyl group, a C₁₋₆ alkoxy group and the like;
(2) a C₆₋₁₄ aryl group optionally substituted by 1 to 3 substituent(s) selected from a halogen atom, a carboxyl group, a C₁₋₆ alkoxy-carbonyl group, a carbamoyl group and the like; or
(3) a C₇₋₁₃ aralkyl group.

Of these, a C₁₋₁₀ alkyl group optionally substituted by 1 to 3 substituent(s) selected from a C₃₋₁₀ cycloalkyl group, a C₁₋₆ alkoxy-carbonyl group, a C₁₋₆ alkoxy group and the like, is preferable.

As the “substituted hydroxy group” of the “optionally substituted hydroxy group” for R¹ or R², those exemplarily recited for X below can be used.

R¹ and R² are each preferably an “optionally substituted hydrocarbon group”, more preferably a C₁₋₁₀ alkyl group optionally substituted by 1 to 3 substituent(s) selected from a C₃₋₁₀ cycloalkyl group, a C₁₋₆ alkoxy-carbonyl group, a C₁₋₆ alkoxy group and the like.

As the “aromatic group” of the “optionally substituted aromatic group” for R³, for example, an aromatic hydrocarbon group, an aromatic heterocyclic group and the like can be mentioned.

As the aromatic hydrocarbon group, for example, a C₋₁₄ aryl group which is exemplarily recited for the “hydrocarbon group” of the “optionally substituted hydrocarbon group” for the aforementioned R¹ or R², and the like can be mentioned.

As the aromatic heterocyclic group, for example, a 5- to 7-membered monocyclic aromatic heterocyclic group containing 1 to 4 heteroatom(s) selected from an oxygen atom, a sulfur atom and a nitrogen atom as a ring-constituting atom, besides carbon atoms, and fused aromatic heterocyclic group can be mentioned. As the fused aromatic heterocyclic group, for example, a group wherein these 5- to 7-membered monocyclic aromatic heterocyclic groups and a 6-membered ring containing 1 or 2 nitrogen atom(s), a benzene ring or a 5-membered ring containing one sulfur atom are fused, and the like can be mentioned.

As preferable examples of the aromatic heterocyclic group, monocyclic aromatic heterocyclic groups such as furyl (e.g., 2-furyl, 3-furyl), thienyl (e.g., 2-thienyl, 3-thienyl), pyridyl (e.g., 2-pyridyl, 3-pyridyl, 4-pyridyl), pyrimidinyl (e.g., 2-pyrimidinyl, 4-pyrimidinyl, 5-pyrimidinyl, 6-pyrimidinyl), pyridazinyl (e.g., 3-pyridazinyl, 4-pyridazinyl), pyrazinyl (e.g., 2-pyrazinyl), pyrrolyl (e.g., 1-pyrrolyl, 2-pyrrolyl, 3-pyrrolyl), imidazolyl (e.g., 1-imidazolyl, 2-imidazolyl, 4-imidazolyl, 5-imidazolyl), pyrazolyl (e.g., 1-pyrazolyl, 3-pyrazolyl, 4-pyrazolyl), thiazolyl (e.g., 2-thiazolyl, 4-thiazolyl, 5-thiazolyl), isothiazolyl, oxazolyl (e.g., 2-oxazolyl, 4-oxazolyl, 5-oxazolyl), isoxazolyl, oxadiazolyl (e.g., 1,2,4-oxadiazol-5-yl, 1,3,4-oxadiazol-2-yl), thiadiazolyl (e.g., 1,3,4-thiadiazol-2-yl), triazolyl (e.g., 1,2,4-triazol-1-yl, 1,2,4-triazol-3-yl, 1,2,3-triazol-1-yl, 1,2,3-triazol-2-yl, 1,2,3-triazol-4-yl), tetrazolyl (e.g., tetrazol-1-yl, tetrazol-5-yl) and the like; fused aromatic heterocyclic groups such as quinolyl (e.g., 2-quinolyl, 3-quinolyl, 4-quinolyl), quinazolyl (e.g., 2-quinazolyl, 4-quinazolyl), quinoxalyl (e.g., 2-quinoxalyl), benzofuryl (e.g., 2-benzofuryl, 3-benzofuryl), benzothienyl (e.g., 2-benzothienyl, 3-benzothienyl), benzoxazolyl (e.g., 2-benzoxazolyl), benzothiazolyl (e.g., 2-benzothiazolyl), benzimidazolyl (e.g., benzimidazol-1-yl, benzimidazol-2-yl), indolyl (e.g., indol-1-yl, indol-3-yl), indazolyl (e.g., 1H-indazol-3-yl), pyrrolopyrazinyl (e.g., 1H-pyrrolo[2,3-b]pyrazin-2-yl, 1H-pyrrolo[2,3-b]pyrazin-6-yl), imidazopyridinyl (e.g., 1H-imidazo[4, 5-b]pyridin-2-yl, 1H-imidazo[4,5-c]pyridin-2-yl), imidazopyrazinyl (e.g., 1H-imidazo[4,5-b]pyrazin-2-yl) and the like, and the like can be mentioned.

The “aromatic group” of the “optionally substituted aromatic group” for R³ is preferably an aromatic hydrocarbon group, more preferably a C₆₋₁₄ aryl group, still more preferably phenyl.

The “aromatic group” of the “optionally substituted aromatic group” for R³ optionally has 1 to 3 substituent(s) at substitutable position(s).

As these substituents, for example, those exemplarily recited for the substituents for the C₃₋₁₀ cycloalkyl group exemplarily recited for the “hydrocarbon group” of the “optionally substituted hydrocarbon group” for the aforementioned R¹ or R² can be mentioned.

The substituents are preferably
a C₁₋₆ alkyl group optionally substituted by 1 to 3 halogen atom(s) (e.g., fluorine, chlorine, bromine, iodine);
a halogen atom (e.g., fluorine, chlorine, bromine, iodine);
a C₁₋₆ alkoxy-carbonyl group;
a carboxyl group;
a hydroxy group;
a C₁₋₆ alkoxy group optionally substituted by 1 to 3 halogen atom(s); and the like, more preferably
a C₁₋₆ alkyl group (e.g., methyl, ethyl) optionally substituted by 1 to 3 halogen atom(s) (e.g., fluorine, chlorine, bromine, iodine);
a halogen atom (e.g., fluorine, chlorine, bromine, iodine); and the like.

The “optionally substituted aromatic group” for R³ is preferably a C₆₋₁₄ aryl group (wherein the C₆₋₁₄ aryl group is preferably a phenyl) optionally substituted by 1 to 3 substituent(s) selected from a C₁₋₆ alkyl group (e.g., methyl, ethyl) optionally substituted by 1 to 3 halogen atom(s) (e.g., fluorine, chlorine, bromine, iodine), a halogen atom (e.g., fluorine, chlorine, bromine, iodine), and the like.

As the “optionally substituted amino group” for R⁴, for example, an amino group optionally substituted by 1 or 2 substituent(s) selected from a C₁₋₁₀ alkyl group, a C₂₋₁₀ alkenyl group, a C₃₋₁₀ cycloalkyl group, a C₃₋₁₀ cycloalkenyl group, a C₆₋₁₄ aryl group, a C₇₋₁₃ aralkyl group and a C₈₋₁₃ arylalkenyl group, each of which is optionally substituted; an acyl group and the like can be mentioned.

As the C₁₋₁₀ alkyl group, C₂₋₁₀ alkenyl group, C₃₋₁₀ cycloalkyl group, C₃₋₁₀ cycloalkenyl group, C₆₋₁₄ aryl group, C₇₋₁₃ aralkyl group and C₈₋₁₃ arylalkenyl group here, those exemplarily recited for the “hydrocarbon group” of the “optionally substituted hydrocarbon group” for the aforementioned R¹ or R² can be used.

These C₁₋₁₀ alkyl group, C₂₋₁₀ alkenyl group, C₃₋₁₀ cycloalkyl group, C₃₋₁₀ cycloalkenyl group, C₆₋₁₄ aryl group, C₇₋₁₃ aralkyl group and C₈₋₁₃ arylalkenyl group each optionally have 1 to 3 substituent(s) at substitutable position(s). As these substituents, for example,
a halogen atom (e.g., fluorine, chlorine, bromine, iodine);
a C₁₋₆ alkoxy-carbonyl group (e.g., methoxycarbonyl, ethoxycarbonyl, tert-butoxycarbonyl);
a C₁₋₆ alkyl-carbonyl group;
a cyano group;
a carbamoyl group optionally mono- or di-substituted by a C₁₋₁₀ alkyl group (e.g., methyl, ethyl, propyl, isopropyl, neopentyl);
a hydroxy group;
a carboxyl group;
and the like can be mentioned.

As the acyl group exemplarily recited for the substituent of the “optionally substituted amino group”, those exemplarily recited for X below can be used. Of these,
(1) a C₁₋₆ alkyl-carbonyl group (e.g., acetyl, isobutanoyl, isopentanoyl);
(2) a C₁₋₆ alkoxy-carbonyl group (e.g., methoxycarbonyl, ethoxycarbonyl, propoxycarbonyl, tert-butoxycarbonyl) optionally substituted by a C₁₋₆ alkoxy-carbonyl group;
(3) a C₃₋₁₀ cycloalkyl-carbonyl group (e.g., cyclopentylcarbonyl, cyclohexylcarbonyl);
(4) a C₆₋₁₄ aryl-carbonyl group (e.g., benzoyl) optionally substituted by 1 to 3 substituent(s) selected from a halogen atom, a cyano group, an optionally halogenated C₁₋₆ alkyl group, a C₁₋₆ alkoxy group, a carboxyl group, a C₁₋₆ alkoxy-carbonyl group, an aromatic heterocyclic group (e.g., tetrazolyl, oxadiazolyl), a non-aromatic heterocyclic group (e.g., oxooxadiazolyl) and a carbamoyl group;
(5) a C₇₋₁₃ aralkyloxy-carbonyl group (e.g., benzyloxycarbonyl) optionally substituted by 1 to 3 substituent(s) selected from a carboxyl group, a C₁₋₆ alkoxy-carbonyl group and a carbamoyl group;
(6) a carbamoyl group;
(7) a mono- or di-C₁₋₆ alkyl-carbamoyl group (e.g., dimethylcarbamoyl);
(8) a C₁₋₆ alkylsulfonyl group (e.g., methylsulfonyl);
(9) a C₆₋₁₄ arylsulfonyl group optionally substituted by a C₁₋₆ alkylsulfonyl group (e.g., phenylsulfonyl, methylsulfonylphenylsulfonyl);
(10) an aromatic heterocyclic (e.g., pyridyl, thiazolyl, oxazolyl, indolyl)-sulfonyl group optionally substituted by 1 to 3 substituent(s) selected from a C₁₋₆ alkyl group and a mono- or di-(C₁₋₆ alkyl-carbonyl)-amino group (e.g., 2-acetylamino-4-methyl-5-thiazolylsulfonyl);
(11) a C₇₋₁₃ aralkyl-carbonyl group (e.g., benzylcarbonyl, phenethylcarbonyl);
(12) a C₈₋₁₃ arylalkenyl-carbonyl group (e.g., styrylcarbonyl);
(13) an aromatic heterocyclic (e.g., furyl, thienyl, oxazolyl, thiazolyl, isoxazolyl, isothiazolyl, pyrazolyl, pyridyl, pyrazinyl, benzofuryl, benzothienyl, quinoxalinyl)-carbonyl group (e.g., furylcarbonyl, thienylcarbonyl, thiazolylcarbonyl, pyrazolylcarbonyl, pyridylcarbonyl, pyrazinylcarbonyl, benzofurylcarbonyl, benzothienylcarbonyl, quinoxalinylcarbonyl) optionally substituted by 1 to 3 substituent(s) selected from a C₁₋₆ alkyl group, a C₆₋₁₄ aryl group, a C₇₋₁₃ aralkyl group, a C₁₋₆ alkoxy group, a carboxyl group, a C₁₋₆ alkoxy-carbonyl group and a carbamoyl group;
(14) a nitrogen-containing heterocyclic (e.g., pyrrolidinyl, piperidinyl, piperazinyl, morpholino, oxopiperazinyl)-carbonyl group optionally substituted by 1 to 3 substituent(s) selected from a C₁₋₆ alkyl group (the C₁₋₆ alkyl group is optionally substituted by 1 to 3 substituent(s) selected from carboxyl group, a C₁₋₆ alkoxy-carbonyl group and a carbamoyl group), a carboxyl group, a C₁₋₆ alkoxy-carbonyl group and a carbamoyl group;
(15) a C₆₋₁₄ aryl-nitrogen-containing heterocyclic (e.g., pyrrolidinyl, piperidinyl, piperazinyl, morpholino)-carbonyl group;
(16) a 4-oxo-4,5,6,7-tetrahydro-1-benzofuranyl-carbonyl group;
(17) a tetrahydropyranylcarbonyl group;
(18) a C₆₋₁₄ aryloxy-carbonyl group optionally substituted by 1 to 3 substituent(s) selected from a carboxyl group, a C₁₋₆ alkoxy-carbonyl group and a carbamoyl group;
(19) a C₇₋₁₃ aralkyl-carbamoyl group (e.g., benzylcarbamoyl);
(20) an aromatic heterocyclic (e.g., pyridyl, thiazolyl, oxazolyl, indolyl)-carbamoyl group (e.g., thiazolylcarbamoyl, oxazolylcarbamoyl) optionally substituted by 1 to 3 substituent(s) selected from a carboxyl group, a C₁₋₆ alkoxy-carbonyl group and a carbamoyl group;
and the like, are preferable.

As preferable examples of the substituted amino group,
(1) a mono- or di-C₁₋₁₀ alkylamino group (e.g., methylamino, dimethylamino, ethylamino, diethylamino, propylamino, dibutylamino);
(2) a mono- or di-C₂₋₁₀ alkenylamino group (e.g., diallylamino);
(3) a mono- or di-C₃₋₁₀ cycloalkylamino group (e.g., cyclohexylamino);
(4) a C₆₋₁₄ arylamino group (e.g., phenylamino);
(5) a mono- or di-(C₁₋₆ alkyl-carbonyl)-amino group (e.g., acetylamino, propionylamino, butanoylamino, isobutanoylamino, isopentanoylamino);
(6) a C₁₋₆ alkoxy-carbonylamino group (e.g., methoxycarbonylamino) optionally substituted by C₁₋₆ alkoxy-carbonyl group;
(7) a carbamoyl-C₁₋₁₀ alkylamino group (e.g., carbamoylmethylamino);
(8) a C₁₋₆ alkoxy-carbonyl-C₁₋₁₀ alkylamino group (e.g., methoxycarbonylmethylamino, ethoxycarbonylmethylamino, tert-butoxycarbonylmethylamino);
(9) a carboxy-C₁₋₁₀ alkylamino group (e.g., carboxymethylamino);
(10) a C₃₋₁₀ cycloalkyl-carbonylamino group (e.g., cyclopentylcarbonylamino, cyclohexylcarbonylamino);
(11) a C₆₋₁₄ aryl-carbonylamino group (e.g., benzoylamino) optionally substituted by 1 to 3 substituent(s) selected from a halogen atom, a cyano group, an optionally halogenated C₁₋₆ alkyl group, a C₁₋₆ alkoxy group, a carboxyl group, a C₁₋₆ alkoxy-carbonyl group, an aromatic heterocyclic group (e.g., tetrazolyl, oxadiazolyl), a non-aromatic heterocyclic group (e.g., oxooxadiazolyl) and a carbamoyl group;
(12) a C₇₋₁₃ aralkyloxy-carbonylamino group (e.g., benzyloxycarbonylamino) optionally substituted by 1 to 3 substituent(s) selected from a carboxyl group, a C₁₋₆ alkoxy-carbonyl group and a carbamoyl group;
(13) a carbamoylamino group;
(14) a mono- or di-C₁₋₆ alkyl-carbamoylamino group (e.g., dimethylcarbamoylamino);
(15) a C₁₋₆ alkylsulfonylamino group (e.g., methylsulfonylamino);
(16) a C₆₋₁₄ arylsulfonylamino group optionally substituted by a C₁₋₆ alkylsulfonyl group (e.g., phenylsulfonylamino, methylsulfonylphenylsulfonylamino);
(17) an aromatic heterocyclic (e.g., pyridyl, thiazolyl, oxazolyl, indolyl)-sulfonylamino group optionally substituted by 1 to 3 substituent(s) selected from a C₁₋₆ alkyl group and a mono- or di-(Cl₁₋₆ alkyl-carbonyl)-amino group (e.g., 2-acetylamino-4-methyl-5-thiazolylsulfonylamino);
(18) a C₇₋₁₃ aralkyl-carbonylamino group (e.g., benzylcarbonylamino, phenethylcarbonylamino);
(19) a C₈₋₁₃ arylalkenyl-carbonylamino group (e.g., styrylcarbonylamino);
(20) an aromatic heterocyclic (e.g., furyl, thienyl, oxazolyl, thiazolyl, isoxazolyl, isothiazolyl, pyrazolyl, pyridyl, pyrazinyl, benzofuryl, benzothienyl, quinoxalinyl)-carbonylamino group optionally substituted by 1 to 3 substituent(s) selected from a C₁₋₆ alkyl group, a C₆₋₁₄ aryl group, a C₇₋₁₃ aralkyl group, a C₁₋₆ alkoxy group, a carboxyl group, a C₁₋₆ alkoxy-carbonyl group and a carbamoyl group;
(21) a nitrogen-containing heterocyclic (e.g., pyrrolidinyl, piperidinyl, piperazinyl, morpholino, oxopiperazinyl)-carbonylamino group optionally substituted by 1 to 3 substituent(s) selected from a C₁₋₆ alkyl group (the C1-6 alkyl group is optionally substituted by 1 to 3 substituent(s) selected from a carboxyl group, a C₁₋₆ alkoxy-carbonyl group and a carbamoyl group), a carboxyl group, a C₁₋₆ alkoxy-carbonyl group and a carbamoyl group;
(22) a C₆₋₁₄ aryl-nitrogen-containing heterocyclic (e.g., pyrrolidinyl, piperidinyl, piperazinyl, morpholino)-carbonylamino group;
(23) a tetrahydropyranylcarbonylamino group;
(24) a 4-oxo-4,5,6,7-tetrahydro-1-benzofuranyl-carbonylamino group;
(25) a C₆₋₁₄ aryloxy-carbonylamino group optionally substituted by 1 to 3 substituent(s) selected from a carboxyl group, a C₁₋₆ alkoxy-carbonyl group and a carbamoyl group;
(26) a C₇₋₁₃ aralkyl-carbamoylamino group (e.g., benzylcarbamoylamino);
(27) an aromatic heterocyclic (e.g., pyridyl, thiazolyl, oxazolyl, indolyl)-carbamoylamino group optionally substituted by 1 to 3 substituent(s) selected from a carboxyl group, a C₁₋₆ alkoxy-carbonyl group and a carbamoyl group;
and the like can be mentioned.

The “optionally substituted amino group” for R⁴ is preferably an amino group optionally mono- or di-substituted by a C₁₋₆ alkyl group (e.g., methyl, ethyl, propyl, isopropyl). R⁴ is particularly preferably an amino group.

As the “divalent chain hydrocarbon group” for L or Q, for example, a divalent chain hydrocarbon group having 1 to 10 carbon atoms can be mentioned. Specific examples include
(1) a C₁₋₁₀ alkylene group (e.g., —CH₂—, —(CH₂)₂—, —(CH₂)₃—, —(CH₂)₄—, —(CH₂)₅—, —(CH₂)₆—, —CHCH₃—, —C(CH₃)₂—, —(CH(CH₃))₂—, —(CH₂)₂C(CH₃)₂—, —(CH₂)₃C(CH₃)₂—);
(2) a C₂₋₁₀ alkenylene group (e.g., —CH═CH—, —CH₂—-CH═CH—, —CH═CH—CH₂—, —CH═CH—CH₂—CH₂—, —C(CH₃)₂—CH═CH—, —CH₂—CH═CH—CH₂—, —CH₂—CH₂—CH═CH—, —CH═CH—CH═CH—, —CH═CH—CH₂—CH₂—CH₂—);
(3) a C₂₋₁₀ alkynylene group (e.g., —C≡C—, —CH₂—C≡C—, —CH₂—C≡C—CH₂—CH₂—) and the like.

The “divalent chain hydrocarbon group” is preferably a C₁₋₁₀ alkylene group or a C₂₋₁₀ alkenylene group, more preferably —CH₂—, —(CH₂)₂—, —CH═CH— and the like.

L is preferably a C₁₋₁₀ alkylene group, more preferably —CH₂— and the like.

Q is preferably a bond, a C₁₋₁₀ alkylene group or a C₂₋₁₀ alkenylene group, more preferably a bond, —CH₂—, —(CH₂)₂—, —CH═CH— and the like. Q is particularly preferably a bond.

As the “acyl group” for X, for example, a group represented by the formula: —COR⁵, —CO—OR⁵, —So₂R⁵, —SOR⁵, —PO₃R⁵R⁶, —CO—-NR^{5a}R^{6a}, —CS—NR^{5a}R^{6a}, [wherein R⁵ and R⁶ are the same or different and each is a hydrogen atom, an optionally substituted hydrocarbon group or an optionally substituted heterocyclic group; R^{5a} and R^{6a} are the same or different and each is a hydrogen atom, an optionally substituted hydrocarbon group or an optionally substituted heterocyclic group, or R^{5a} and R^{6a} may form an optionally substituted nitrogen-containing heterocycle together with the adjacent nitrogen atom], and the like can be mentioned.

As the “optionally substituted hydrocarbon group” for R⁵, R⁶, R^{5a} or R^{6a}, those exemplarily recited for the aforementioned R¹ or R² can be used.

As the “heterocyclic group” of the “optionally substituted heterocyclic group” for R⁵, R⁶, R^{5a} or R^{6a}, an aromatic heterocyclic group and a non-aromatic heterocyclic group can be mentioned.

As the aromatic heterocyclic group, those exemplarily recited for the “aromatic group” of the “optionally substituted aromatic group” for the aforementioned R³ can be mentioned.

As the non-aromatic heterocyclic group, for example, a 5- to 7-membered monocyclic non-aromatic heterocyclic group containing 1 to 4 heteroatom(s) selected from an oxygen atom, a sulfur atom and a nitrogen atom as a ring-constituting atom, besides carbon atoms, and a fused non-aromatic heterocyclic group can be mentioned. As the fused non-aromatic heterocyclic group, for example, a group wherein these 5- to 7-membered monocyclic non-aromatic heterocyclic groups and a 6-membered ring containing 1 or 2 nitrogen atom(s), a benzene ring or a 5-membered ring containing one sulfur atom are fused, and the like can be mentioned.

As preferable examples of the non-aromatic heterocyclic group, pyrrolidinyl (e.g., 1-pyrrolidinyl), piperidinyl (e.g., piperidino), morpholinyl (e.g., morpholino), thiomorpholinyl (e.g., thiomorpholino), piperazinyl (e.g., 1-piperazinyl), hexamethyleniminyl (e.g., hexamethylenimin-1-yl), oxazolidinyl (e.g., oxazolidin-3-yl), thiazolidinyl (e.g., thiazolidin-3-yl), imidazolidinyl (e.g., imidazolidin-3-yl), oxoimidazolidinyl (e.g., 2-oxoimidazolidin-1-yl), dioxoimidazolidinyl (e.g., 2,4-dioxoimidazolidin-3-yl), dioxooxazolidinyl (e.g., 2,4-dioxooxazolidin-3-yl, 2,4-dioxooxazolidin-5-yl, 2,4-dioxooxazolidin-1-yl), dioxothiazolidinyl (e.g., 2,4-dioxothiazolidin-3-yl, 2,4-dioxothiazolidin-5-yl), dioxoisoindolyl (e.g., 1,3-dioxoisoindol-2-yl), oxooxadiazolyl (e.g., 5-oxooxadiazol-3-yl), oxothiadiazolyl (e.g., 5-oxothiadiazol-3-yl), oxopiperazinyl (e.g., 3-oxopiperazin-1-yl), dioxopiperazinyl (e.g., 2,3-dioxopiperazin-1-yl, 2,5-dioxopiperazin-1-yl), oxodioxolyl (e.g., 2-oxo-1,3-dioxol-4-yl), oxodioxolanyl (e.g., 2-oxo-1,3-dioxolan-4-yl), oxo-2-benzofuranyl (e.g., 3-oxo-2-benzofuran-1-yl), oxodihydrooxadiazolyl (e.g., 5-oxo-4,5-dihydro-1,2,4-oxadiazol-3-yl), 4-oxo-2-thioxo-1,3-thiazolidin-5-yl, 4-oxo-2-thioxo-1,3-oxazolidin-5-yl, tetrahydropyranyl (e.g., 4-tetrahydropyranyl), 4-oxo-4,5,6,7-tetrahydro-1-benzofuranyl (e.g., 4-oxo-4,5,6,7-tetrahydro-1-benzofuran-3-yl), 1,3(2H,5H)-dioxo-tetrahydroimidazo[1,5-a]pyridinyl, 1,3(2H,5H)-dioxo-10,10a-dihydroimidazo[1,5-b]isoquinolinyl and the like can be mentioned.

The “heterocyclic group” of the “optionally substituted heterocyclic group” for R⁵, R⁶, R^{5a} or R^{6a} optionally has 1 to 3 substituent(s) at substitutable position(s).

As these substituents, for example, those exemplarily recited for the substituents for the C₃₋₁₀ cycloalkyl group exemplarily recited for the “hydrocarbon group” of the “optionally substituted hydrocarbon group” for the aforementioned R¹ or R² can be mentioned.

The substituents are preferably a C₁₋₆ alkyl group (e.g., methyl, ethyl) optionally substituted by 1 to 3 halogen atom(s) (e.g., fluorine, chlorine, bromine, iodine);
a halogen atom (e.g., fluorine, chlorine, bromine, iodine);
a C₆₋₁₄ aryl group;
a C₇₋₁₃ aralkyl group;
a hydroxy group;
a C₁₋₆ alkoxy group;
a carboxyl group;
a C₁₋₆ alkoxy-carbonyl group;
a carbamoyl group;
a C₁₋₆ alkyl group substituted by 1 to 3 substituent(s) selected from a carboxyl group, a C₁₋₆ alkoxy-carbonyl group and a carbamoyl group;
a mono- or di-(C₁₋₆ alkyl-carbonyl)-amino group;
and the like.

As the “nitrogen-containing heterocycle” of the “optionally substituted nitrogen-containing heterocycle” formed by R^{5a} and R^{6a} together with the adjacent nitrogen atom, for example, a 5- to 7-membered nitrogen-containing heterocycle containing at least one nitrogen atom and optionally further containing 1 to 2 heteroatom(s) selected from an oxygen atom, a sulfur atom and a nitrogen atom as a ring-constituting atom, besides carbon atoms can be mentioned. As preferable examples of the “nitrogen-containing heterocycle”, pyrrolidine, imidazolidine, pyrazolidine, piperidine, piperazine, morpholine, thiomorpholine, oxopiperazine and the like can be mentioned.

The nitrogen-containing heterocycle optionally has 1 to 3 (preferably 1 or 2) substituent(s) at substitutable position(s). As these substituents,
a hydroxy group;
a C₁₋₆ alkyl group optionally substituted by 1 to 3 halogen atom(s) (e.g., fluorine, chlorine, bromine, iodine);
a C₇₋₁₃ aralkyl group (e.g., benzyl, diphenylmethyl) optionally substituted by 1 to 3 halogen atom(s) (e.g., fluorine, chlorine, bromine, iodine);
a C₆₋₁₄ aryl group (e.g., phenyl) optionally substituted by 1 to 3 halogen atom(s) (e.g., fluorine, chlorine, bromine, iodine);
a C₁₋₆ alkoxy-carbonyl group (e.g., methoxycarbonyl, ethoxycarbonyl);
a C₁₋₆ alkyl group substituted by 1 to 3 substituent(s) selected from a carboxyl group, a C₁₋₆ alkoxy-carbonyl group and a carbamoyl group;
a carboxyl group;
a carbamoyl group;
and the like can be mentioned.

As preferable examples of the “acyl group”,
(1) a formyl group;
(2) a carboxyl group;
(3) a carbamoyl group;
(4) a C₁₋₆ alkyl-carbonyl group (e.g., acetyl, isobutanoyl, isopentanoyl);
(5) a C₁₋₆ alkoxy-carbonyl group optionally substituted by 1 to 3 substituent(s) selected from a carboxyl group, a carbamoyl group, a thiocarbamoyl group, a C₁₋₆ alkoxy-carbonyl group and a C₁₋₆ alkyl-carbonyloxy group (e.g., methoxycarbonyl, ethoxycarbonyl, propoxycarbonyl, tert-butoxycarbonyl; carboxymethoxycarbonyl, carboxyethoxycarbonyl, carboxybutoxycarbonyl; carbamoylmethoxycarbonyl; thiocarbamoylmethoxycarbonyl; ethoxycarbonylmethoxycarbonyl, ethoxycarbonylethoxycarbonyl, methoxycarbonylbutoxycarbonyl, ethoxycarbonylbutoxycarbonyl; tert-butylcarbonyloxymethoxycarbonyl);
(6) an aromatic heterocyclic (e.g., furyl, thienyl, pyridyl, thiazolyl, oxazolyl, pyrazinyl, indolyl)-C₁₋₆ alkoxy-carbonyl group optionally substituted by 1 to 3 substituent(s) selected from a carboxyl group, a carbamoyl group, a thiocarbamoyl group and a C₁₋₆alkoxy-carbonyl group (e.g., pyridylmethoxycarbonyl; carboxythiazolylmethoxycarbonyl; carbamoylthiazolylmethoxycarbonyl; ethoxycarbonylthiazolylmethoxycarbonyl);
(7) a non-aromatic heterocyclic (e.g., oxodioxolyl, oxodioxolanyl, oxo-2-benzofuranyl)-C₁₋₆ alkoxy-carbonyl group optionally substituted by a C₁₋₆ alkyl group (e.g., methyloxodioxolylmethoxycarbonyl, oxo-2-benzofuranylethoxycarbonyl);
(8) a C₃₋₁₀ cycloalkyl-carbonyl group (e.g., cyclopentylcarbonyl, cyclohexylcarbonyl);
(9) a C₆₋₁₄ aryl-carbonyl group (e.g., benzoyl, 1-naphthoyl, 2-naphthoyl) optionally substituted by 1 to 3 substituent(s) selected from a halogen atom, a cyano group, an optionally halogenated C₁₋₆alkyl group (i.e., C₁₋₆ alkyl group optionally substituted by 1 to 3 halogen atom(s) (e.g., fluorine, chlorine, bromine, iodine)), a C₁₋₆ alkoxy group, a carboxyl group, a C₁₋₆ alkoxy-carbonyl group, an aromatic heterocyclic group (e.g., tetrazolyl, oxadiazolyl), a non-aromatic heterocyclic group (e.g., oxooxadiazolyl) and a carbamoyl group;
(10) a C₆₋₁₄ aryloxy-carbonyl group (e.g., phenyloxycarbonyl, naphthyloxycarbonyl) optionally substituted by 1 to 3 substituent(s) selected from a carboxyl group, a C₁₋₆ alkoxy-carbonyl group and a carbamoyl group;
(11) a C₇₋₁₃ aralkyloxy-carbonyl group optionally substituted by 1 to 3 substituent(s) selected from a carboxyl group, a carbamoyl group, a thiocarbamoyl group, a C₁₋₆ alkoxy-carbonyl group, a halogen atom, a cyano group, a nitro group, a C₁₋₆ alkoxy group, a C₁₋₆ alkylsulfonyl group and a C₁₋₆ alkyl group (the C₁₋₆ alkyl group is optionally substituted by 1 to 3 substituent(s) selected from a halogen atom, a carboxyl group, a C₁₋₆ alkoxy-carbonyl group and a carbamoyl group) (e.g., benzyloxycarbonyl, phenethyloxycarbonyl; carboxybenzyloxycarbonyl; methoxycarbonylbenzyloxycarbonyl, biphenylylmethoxycarbonyl);
(12) a carbamoyl group mono- or di-substituted by a C₁₋₆ alkyl group optionally substituted by 1 to 3 substituent(s) selected from halogen atoms (e.g., fluorine, chlorine, bromine, iodine) and a C₁₋₆ alkoxy group (e.g., methylcarbamoyl, ethylcarbamoyl, dimethylcarbamoyl, diethylcarbamoyl, ethylmethylcarbamoyl, propylcarbamoyl, isopropylcarbamoyl, butylcarbamoyl, isobutylcarbamoyl, trifluoroethylcarbamoyl, N-methoxyethyl-N-methylcarbamoyl);
(13) a carbamoyl-C₁₋₆ alkyl-carbamoyl group optionally mono- or di-substituted by a C₁₋₆ alkyl group optionally substituted by 1 to 3 halogen atom(s) (e.g., fluorine, chlorine, bromine, iodine) (e.g., carbamoylmethylcarbamoyl, carbamoylethylcarbamoyl, dimethylcarbamoylmethylcarbamoyl, dimethylcarbamoylethylcarbamoyl);
(14) a C₁₋₆ alkoxy-carbonyl-C₁₋₆ alkyl-carbamoyl group optionally substituted by a C₁₋₆ alkyl group (e.g., methoxycarbonylmethylcarbamoyl, ethoxycarbonylethylcarbamoyl, N-ethoxycarbonylmethyl-N-methylcarbamoyl);
(15) a C₆₋₁₄ aryl-carbamoyl group (e.g., phenylcarbamoyl) optionally substituted by 1 to 3 substituent(s) selected from an amino group optionally mono- or di-substituted by a C₁₋₆ alkyl group, a carboxyl group, a C₁₋₆ alkoxy-carbonyl group, an aromatic heterocyclic group (e.g., tetrazolyl, oxadiazolyl), a non-aromatic heterocyclic group (e.g., oxooxadiazolyl) and a carbamoyl group;
(16) a mono- or di-C₃₋₁₀ cycloalkyl-carbamoyl group optionally substituted by a C₁₋₆ alkyl group (e.g., cyclopropylcarbamoyl, cyclopentylcarbamoyl, dicyclohexylcarbamoyl, N-cyclohexyl-N-methylcarbamoyl);
(17) a C₇₋₁₃ aralkyl-carbamoyl group optionally substituted by 1 to 3 substituent(s) selected from a halogen atom (e.g., fluorine, chlorine, bromine, iodine), a hydroxy group, a carboxyl group, a C₁₋₆ alkoxy-carbonyl group and a C₁₋₆ alkyl group (e.g., benzylcarbamoyl, phenethylcarbamoyl, phenylpropylcarbamoyl, hydroxyphenethylcarbamoyl, chlorobenzylcarbamoyl, methoxycarbonylbenzylcarbamoyl, N-benzyl-N-methylcarbamoyl);
(18) an aromatic heterocyclic (e.g., pyridyl, thienyl, furyl, thiazolyl, oxazolyl, indolyl)-C₁₋₆ alkyl-carbamoyl group (e.g., indolylethylcarbamoyl, pyridylmethylcarbamoyl, thienylmethylcarbamoyl, thiazolylmethylcarbamoyl) optionally substituted by 1 to 3 substituent(s) selected from a carboxyl group, a carbamoyl group and a C₁₋₆ alkoxy-carbonyl group;
(19) a C₁₋₆ alkylsulfonyl group optionally substituted by 1 to 3 substituent(s) selected from a carboxyl group, a carbamoyl group and a C₁₋₆ alkoxy-carbonyl group (e.g., methylsulfonyl, carboxymethylsulfonyl);
(20) a C₆₋₁₄ arylsulfonyl group optionally substituted by 1 to 3 substituent(s) selected from a C₁₋₆ alkyl group, a carboxyl group, a carbamoyl group, a thiocarbamoyl group, a C₁₋₆ alkoxy-carbonyl group and a C₁₋₆ alkylsulfonyl group (e.g., phenylsulfonyl; methylphenylsulfonyl; carboxyphenylsulfonyl; methoxycarbonylphenylsulfonyl; methylsulfonylphenylsulfonyl);
(21) a nitrogen-containing heterocyclic (e.g., pyrrolidinyl, piperidinyl, piperazinyl, morpholino, oxopiperazinyl)-carbonyl group optionally substituted by 1 to 3 substituent(s) selected from a hydroxy group, a C₁₋₆ alkyl group (the C₁₋₆ alkyl group is optionally substituted by 1 to 3 substituent(s) selected from a carboxyl group, a C₁₋₆ alkoxy-carbonyl group and a carbamoyl group), a carboxyl group, a C₁₋₆ alkoxy-carbonyl group and a carbamoyl group (e.g., pyrrolidinylcarbonyl, piperidinylcarbonyl, piperazinylcarbonyl, oxopiperazinylcarbonyl, morpholinocarbonyl, methoxycarbonylpyrrolidinylcarbonyl);
(22) a C₆₋₁₄ aryl-nitrogen-containing heterocyclic (e.g., pyrrolidinyl, piperidinyl, piperazinyl, morpholino)-carbonyl group (e.g., phenylpiperazinylcarbonyl, phenylpiperidinylcarbonyl) optionally substituted by 1 to 3 halogen atom(s) (e.g., fluorine, chlorine, bromine, iodine);
(23) a C₇₋₁₃ aralkyl-nitrogen-containing heterocyclic (e.g., pyrrolidinyl, piperidinyl, piperazinyl, morpholino)-carbonyl group (e.g., benzylpiperazinylcarbonyl) optionally substituted by 1 to 3 halogen atom(s) (e.g., fluorine, chlorine, bromine, iodine);
(24) an aromatic heterocyclic (e.g., pyridyl, thiazolyl, oxazolyl, indolyl)-sulfonyl group optionally substituted by 1 to 3 substituent(s) selected from a C₁₋₆ alkyl group and a mono- or di-(C₁₋₆ alkyl-carbonyl)-amino group (e.g., 2-acetylamino-4-methyl-5-thiazolylsulfonyl);
(25) a non-aromatic heterocyclic (e.g., oxodioxolyl, oxodioxolanyl, oxo-2-benzofuranyl)oxy-carbonyl group (e.g., oxodioxolanyloxycarbonyl, oxo-2-benzofuranyloxycarbonyl);
(26) a C₁₋₆ alkylsulfinyl group (e.g., methylsulfinyl);
(27) a thiocarbamoyl group;
(28) a phosphono group optionally mono- or di-substituted by a C₁₋₆ alkyl group (e.g., dimethyl phosphono, diethyl phosphono);
(29) a C₇₋₁₃ aralkyl-carbonyl group (e.g., benzylcarbonyl, phenethylcarbonyl);
(30) a C₈₋₁₃ arylalkenyl-carbonyl group (e.g., styrylcarbonyl);
(31) an aromatic heterocyclic (e.g., furyl, thienyl, oxazolyl, thiazolyl, isoxazolyl, isothiazolyl, pyrazolyl, pyridyl, pyrazinyl, benzofuryl, benzothienyl, quinoxalinyl)-carbonyl group (e.g., furylcarbonyl, thienylcarbonyl, thiazolylcarbonyl, pyrazolylcarbonyl, pyridylcarbonyl, pyrazinylcarbonyl, benzofurylcarbonyl, benzothienylcarbonyl, quinoxalinylcarbonyl) optionally substituted by 1 to 3 substituent(s) selected from a C₁₋₆ alkyl group, a C₆₋₁₄ aryl group, a C₇₋₁₃ aralkyl group, a C₁₋₆ alkoxy group, a carboxyl group, a C₁₋₆ alkoxy-carbonyl group and a carbamoyl group;
(32) a tetrahydropyranylcarbonyl group;
(33) a 4-oxo-4,5,6,7-tetrahydro-1-benzofuranyl-carbonyl group;
(34) a C₃₋₁₀ cycloalkyl-C₁₋₆ alkoxy-carbonyl group (e.g., cyclohexylmethoxycarbonyl) optionally substituted by 1 to 3 substituent(s) selected from a carboxyl group, a C₁₋₆ alkoxy-carbonyl group and a carbamoyl group;
(35) an aromatic heterocyclic (e.g., thienyl, furyl, pyridyl, oxazolyl, thiazolyl, tetrazolyl, pyridyl, quinolyl, indolyl)-C₇₋₁₃ aralkyloxy-carbonyl group (e.g., tetrazolylbenzyloxycarbonyl);
(36) an aromatic heterocyclic (e.g., thienyl, furyl, pyridyl, thiazolyl, oxazolyl, indolyl)-carbamoyl group (e.g., thienylcarbamoyl, furylcarbamoyl, thiazolylcarbamoyl, oxazolylcarbamoyl) optionally substituted by 1 to 3 substituent(s) selected from a carboxyl group, a C₁₋₆ alkoxy-carbonyl group and a carbamoyl group;
and the like can be mentioned.

The “acyl group” for X is preferably
(1) a carboxyl group;
(2) a carbamoyl group;
(3) a C₁₋₆ alkoxy-carbonyl group optionally substituted by 1 to 3 substituent(s) selected from a carboxyl group, a carbamoyl group, a thiocarbamoyl group, a C₁₋₆ alkoxy-carbonyl group and a C₁₋₆ alkyl-carbonyloxy group (e.g., methoxycarbonyl, ethoxycarbonyl, propoxycarbonyl, tert-butoxycarbonyl; carboxymethoxycarbonyl, carboxyethoxycarbonyl, carboxybutoxycarbonyl; carbamoylmethoxycarbonyl; thiocarbamoylmethoxycarbonyl; ethoxycarbonylmethoxycarbonyl, ethoxycarbonylethoxycarbonyl, methoxycarbonylbutoxycarbonyl, ethoxycarbonylbutoxycarbonyl; tert-butylcarbonyloxymethoxycarbonyl);
(4) a carbamoyl group mono- or di-substituted by a C₁₋₆ alkyl group optionally substituted by 1 to 3 substituent(s) selected from a halogen atom and a C₁₋₆ alkoxy group (e.g., methylcarbamoyl, ethylcarbamoyl, dimethylcarbamoyl, diethylcarbamoyl, ethylmethylcarbamoyl, propylcarbamoyl, isopropylcarbamoyl, butylcarbamoyl, isobutylcarbamoyl, trifluoroethylcarbamoyl, N-methoxyethyl-N-methylcarbamoyl);
(5) a carbamoyl-C₁₋₆ alkyl-carbamoyl group optionally mono- or di-substituted by a C₁₋₆ alkyl group optionally substituted by 1 to 3 halogen atom(s) (e.g., carbamoylmethylcarbamoyl, carbamoylethylcarbamoyl, dimethylcarbamoylmethylcarbamoyl, dimethylcarbamoylethylcarbamoyl);
and the like. Of these, a carboxyl group is preferable.

As the “substituted hydroxy group” for X, for example, a hydroxy group substituted by a substituent selected from a C₁₋₁₀ alkyl group, a C₂₋₁₀ alkenyl group, a C₃₋₁₀ cycloalkyl group, a C₃₋₁₀ cycloalkenyl group, a C₆₋₁₄ aryl group, a C₇₋₁₃ aralkyl group, a C₈₋₁₃ arylalkenyl group, a C₁₋₆ alkyl-carbonyl group (e.g., acetyl, isobutanoyl, isopentanoyl), a 5- or 6-membered aromatic heterocyclic group (e.g., furyl, thienyl, thiazolyl, oxazolyl, imidazolyl, triazolyl, pyrazolyl, pyrimidinyl), a fused aromatic heterocyclic group (e.g., indolyl) and the like, each of which is optionally substituted, can be mentioned.

As the C₁₋₁₀ alkyl group, a C₂₋₁₀ alkenyl group, a C₃₋₁₀ cycloalkyl group, a C₃₋₁₀ cycloalkenyl group, a C₆₋₁₄ aryl group, a C₇₋₁₃ aralkyl group and a C₈₋₁₃ arylalkenyl group here, those exemplarily recited for the “hydrocarbon group” of the “optionally substituted hydrocarbon group” for the aforementioned R¹ or R² can be used.

The aforementioned C₁₋₁₀ alkyl group, C₂₋₁₀ alkenyl group, C₃₋₁₀ cycloalkyl group, C₃₋₁₀ cycloalkenyl group, C₆₋₁₄ aryl group, C₇₋₁₃ aralkyl group, C₈₋₁₃ arylalkenyl group, C₁₋₆ alkyl-carbonyl group, 5- or 6-membered aromatic heterocyclic group and fused aromatic heterocyclic group each optionally have 1 to 3 substituent(s) at substitutable position(s). As these substituents, for example,
a halogen atom (e.g., fluorine, chlorine, bromine, iodine);
a hydroxy group;
a cyano group;
a C₁₋₆ alkyl group optionally substituted by 1 or 2 substituent(s) selected from a halogen atom (e.g., fluorine, chlorine, bromine, iodine), a carboxyl group, a C₁₋₆ alkoxy-carbonyl group (e.g., methoxycarbonyl, tert-butoxycarbonyl) and a carbamoyl group;
a C₁₋₆ alkoxy group optionally substituted by 1 or 2 substituent(s) selected from a halogen atom (e.g., fluorine, chlorine, bromine, iodine), a carboxyl group and a C₁₋₆ alkoxy-carbonyl group (e.g., tert-butoxycarbonyl);
a C₁₋₆ alkylthio group (e.g., methylthio, ethylthio);
a C₁₋₆ alkyl-carbonyl group;
a carboxyl group;
a C₁₋₆ alkoxy-carbonyl group (e.g., methoxycarbonyl, ethoxycarbonyl);
a carbamoyl group optionally mono- or di-substituted by a C₁₋₁₀ alkyl group (e.g., methyl, ethyl, propyl, isopropyl, neopentyl);
an amino group optionally mono- or di-substituted by a C₁₋₁₀ alkyl group (e.g., methyl, ethyl, propyl, isopropyl, neopentyl);
a C₁₋₆ alkyl-carbonylamino group;
an aromatic heterocyclic group (e.g., furyl, thienyl, oxazolyl, thiazolyl, isoxazolyl, tetrazolyl, oxadiazolyl, thiadiazolyl, pyridyl) optionally substituted by 1 to 3 substituent(s) selected from a C₁₋₆ alkyl group (e.g., methyl, ethyl), carboxyl group, a C₁₋₆ alkoxy-carbonyl group (e.g., methoxycarbonyl, ethoxycarbonyl) and a carbamoyl group;
a C₁₋₆ alkylsulfinyl group (e.g., methylsulfinyl);
a C₁₋₆ alkylsulfonyl group (e.g., methylsulfonyl);
and the like can be mentioned.

As preferable examples of the “substituted hydroxy group”,
(1) a C₁₋₆ alkyl-carbonyloxy group;
(2) a C₁₋₁₀ alkoxy group optionally substituted by 1 to 3 substituent(s) selected from a hydroxy group, a carboxyl group, a carbamoyl group and a C₁₋₆ alkoxy-carbonyl group;
(3) a C₆₋₁₄ aryloxy group optionally substituted by 1 to 3 substituent(s) selected from a halogen atom, a carboxyl group, a C₁₋₆ alkoxy-carbonyl group, a C₁₋₆ alkylthio group, a carbamoyl group, a C₁₋₆ alkoxy group, a C₁₋₆ alkylsulfonyl group, a C₁₋₆ alkylsulfinyl group and a C₁₋₆ alkyl group (the C₁₋₆ alkyl group is optionally substituted by 1 or 2 substituent(s) selected from a carboxyl group, a C₁₋₆ alkoxy-carbonyl group and a carbamoyl group);
(4) a 5- or 6-membered aromatic heterocyclyloxy group (preferably thienyloxy, thiazolyloxy, oxazolyloxy, imidazolyloxy, triazolyloxy, pyrazolyloxy, pyridyloxy, pyrimidinyloxy) optionally substituted by 1 to 3 substituent(s) selected from a C₁₋₆ alkyl group (the C₁₋₆ alkyl group is optionally substituted by 1 to 2 substituent(s) selected from a carboxyl group, a C₁₋₆ alkoxy-carbonyl group and a carbamoyl group), a carboxyl group, a C₁₋₆ alkoxy-carbonyl group and a carbamoyl group;
(5) a fused aromatic heterocyclyloxy group (preferably indolyloxy) optionally substituted by 1 to 3 substituent(s) selected from a carboxyl group, a C₁₋₆ alkoxy-carbonyl group and a carbamoyl group;
(6) an aromatic heterocyclic (preferably pyridyl)-C₁₋₆ alkoxy group optionally substituted by 1 to 3 substituent(s) selected from a carboxyl group, a C₁₋₆ alkoxy-carbonyl group and a carbamoyl group;
(7) an aromatic heterocyclic (preferably tetrazolyl)-C₆₋₁₄ aryloxy group;
and the like can be mentioned.

As the “optionally substituted thiol group” for X, for example, a thiol group optionally substituted by a substituent selected from a C₁₋₁₀ alkyl group, a C₂₋₁₀ alkenyl group, a C₃₋₁₀ cycloalkyl group, a C₃₋₁₀ cycloalkenyl group, a C₆₋₁₄ aryl group, a C₇₋₁₃ aralkyl group, a C₈₋₁₃ arylalkenyl group, a C₁₋₆ alkyl-carbonyl group (e.g., acetyl, isobutanoyl, isopentanoyl), a 5- or 6-membered aromatic heterocyclic group (e.g., furyl, thienyl, thiazolyl, oxazolyl, imidazolyl, triazolyl, pyrazolyl, pyrimidinyl), a fused aromatic heterocyclic group (e.g., indolyl) and the like, each of which is optionally substituted, can be mentioned.

As the C₁₋₁₀ alkyl group, C₂₋₁₀ alkenyl group, C₃₋₁₀ cycloalkyl group, C₃₋₁₀ cycloalkenyl group, C₆₋₁₄ aryl group, C₇₋₁₃ aralkyl group and C₈₋₁₃ arylalkenyl group here, those exemplarily recited for the “hydrocarbon group” of the “optionally substituted hydrocarbon group” for the aforementioned R¹ or R² can be used.

The aforementioned C₁₋₁₀ alkyl group, C₂₋₁₀ alkenyl group, C₃₋₁₀ cycloalkyl group, C₃₋₁₀ cycloalkenyl group, C₆₋₁₄ aryl group, C₇₋₁₃ aralkyl group, C₈₋₁₃ arylalkenyl group, C₁₋₆ alkyl-carbonyl group, 5- or 6-membered aromatic heterocyclic group and fused aromatic heterocyclic group each optionally have 1 to 3 substituent(s) at substitutable position(s). As these substituents, the substituents for the C₁₋₁₀ alkyl group and the like for the “substituted hydroxy group” for the aforementioned X can be used.

As preferable examples of the “optionally substituted thiol group”,
(1) a C₁₋₆ alkylthio group optionally substituted by 1 to 3 substituent(s) selected from a hydroxy group, a carboxyl group, a carbamoyl group and a C₁₋₆ alkoxy-carbonyl group;
(2) a C₆₋₁₄ arylthio group optionally substituted by 1 to 3 substituent(s) selected from a carboxyl group, a C₁₋₆ alkoxy-carbonyl group, a C₁₋₆ alkylthio group and a carbamoyl group;
(3) a 5 or 6-membered aromatic heterocyclylthio group (preferably thienylthio, thiazolylthio, oxazolylthio, imidazolylthio, triazolylthio, pyrazolylthio, pyridylthio, pyrimidinylthio) optionally substituted by 1 to 3 substituent(s) selected from a C₁₋₆ alkyl group, a carboxyl group, a C₁₋₆ alkoxy-carbonyl group and a carbamoyl group; and the like can be mentioned.

As the “optionally substituted amino group” for X, those exemplarily recited for the aforementioned R⁴ can be used.

As the “cyclic group” of the “optionally substituted cyclic group” for X, for example, an aromatic hydrocarbon group, a non-aromatic cyclic hydrocarbon group, an aromatic heterocyclic group, a non-aromatic heterocyclic group and the like can be mentioned.

As the aromatic hydrocarbon group and the aromatic heterocyclic group, those exemplarily recited for the “aromatic group” of the “optionally substituted aromatic group” for the aforementioned R³ can be used.

In addition, as the non-aromatic heterocyclic group, those exemplarily recited for the “heterocyclic group” of the “optionally substituted heterocyclic group” for the aforementioned R⁵ can be used.

As the non-aromatic cyclic hydrocarbon group, for example, a C₃₋₁₀ cycloalkyl group, a C₃₋₁₀ cycloalkenyl group, a C₄₋₁₀ cycloalkadienyl group and the like, each of which is optionally fused with a benzene ring, can be mentioned.

As the C₃₋₁₀ cycloalkyl group, C₃₋₁₀ cycloalkenyl group and C₄₋₁₀ cycloalkadienyl group here, those exemplarily recited for the “hydrocarbon group” of the “optionally substituted hydrocarbon group” for the aforementioned R¹ or R² can be used.

The “cyclic group” of the “optionally substituted cyclic group” for X optionally has 1 to 3 substituent(s) at substitutable position(s).

As these substituents, for example, those exemplarily recited for the substituents for the C₃₋₁₀ cycloalkyl group exemplarily recited for the “hydrocarbon group” of the “optionally substituted hydrocarbon group” for the aforementioned R¹ or R² can be mentioned.

The substituents are preferably
a C₁₋₆ alkyl group (e.g., methyl, ethyl) optionally substituted by 1 to 3 substituent(s) selected from a halogen atom (e.g., fluorine, chlorine, bromine, iodine), a carbamoyl group, a carboxyl group and a C₁₋₆ alkoxy-carbonyl group (e.g., methoxycarbonyl, ethoxycarbonyl);
a halogen atom (e.g., fluorine, chlorine, bromine, iodine);
a carboxyl group;
a C₁₋₆ alkoxy-carbonyl group;
a carbamoyl group;
and the like.

X is preferably an acyl group, a substituted hydroxy group, an optionally substituted thiol group or an optionally substituted amino group, more preferably an acyl group. Of these,
(1) a carboxyl group;
(2) a carbamoyl group;
(3) a C₁₋₆ alkoxy-carbonyl group optionally substituted by 1 to 3 substituent(s) selected from a carboxyl group, a carbamoyl group, a thiocarbamoyl group, a C₁₋₆ alkoxy-carbonyl group and a C₁₋₆ alkyl-carbonyloxy group;
(4) a carbamoyl group mono- or di-substituted by a C₁₋₆ alkyl group optionally substituted by 1 to 3 substituent(s) selected from a halogen atom and a C₁₋₆ alkoxy group;
(5) a carbamoyl-C₁₋₆ alkyl-carbamoyl group optionally mono- or di-substituted by a C₁₋₆ alkyl group optionally substituted by 1 to 3 halogen atom(s);
and the like are preferable, and a carboxyl group is particularly preferable.

Of compound (I), when X is an ethoxycarbonyl group, then Q is a divalent chain hydrocarbon group.

Moreover, compound (I) does not comprise 2,6-diisopropyl-3-methylaminomethyl-4-(4-fluorophenyl)-5-pentylpyridine [this compound is also designated as {[4-(4-fluorophenyl)-2,6-diisopropyl-5-pentylpyridin-3-yl]methyl}methylamine];
2,6-diisopropyl-3-aminomethyl-4-(4-fluorophenyl)-5-pentylpyridine [this compound is also designated as {[4-(4-fluorophenyl)-2,6-diisopropyl-5-pentylpyridin-3-yl]methyl}amine];
2,6-diisopropyl-3-(dimethylamino)methyl-4-(4-fluorophenyl)-5-pentylpyridine [this compound is also designated as 1-[4-(4-fluorophenyl)-2,6-diisopropyl-5-pentylpyridin-3-yl]-N,N-dimethylmethaneamine];
2,6-diisopropyl-3-(ethylamino)methyl-4-(4-fluorophenyl)-5-pentylpyridine [this compound is also designated as N-{[4-(4-fluorophenyl)-2,6-diisopropyl-5-pentylpyridin-3-yl]methyl}ethaneamine]; and
3-(tert-butyldimethylsilyloxymethyl)-2,6-diisopropyl-4-(4-fluorophenyl)-5-(indolyl-5-aminomethyl)pyridine [this compound is also designated as N-{[5-({[tert-butyl(dimethyl)silyl]oxy}methyl)-4-(4-fluorophenyl)-2,6-diisopropylpyridin-3-yl]methyl}-1H-indol-5-amine].

As preferable examples of compound (I), the following compounds can be mentioned.

[Compound A]

A compound wherein R¹ and R² are the same or different and each is a C₁₋₁₀ alkyl group (preferably methyl, ethyl, propyl, isopropyl, butyl, isobutyl, neopentyl) optionally substituted by 1 to 3 substituent(s) selected from a C₃₋₁₀ cycloalkyl group (preferably cyclopropyl), a C₁₋₆ alkoxy-carbonyl group (preferably methoxycarbonyl) and the like;
R³ is a C₆₋₁₄ aryl group (the C₆₋₁₄ aryl group is preferably phenyl) optionally substituted by 1 to 3 substituent(s) selected from a C₁₋₆ alkyl group (e.g., methyl, ethyl) optionally substituted by 1 to 3 halogen atom(s) (e.g., fluorine, chlorine, bromine, iodine), a halogen atom (e.g., fluorine, chlorine, bromine, iodine) and the like;
R⁴ is an amino group optionally mono- or di-substituted by a C₁₋₆ alkyl group (e.g., methyl, ethyl, propyl, isopropyl);
L is a C₁₋₁₀ alkylene group (preferably —CH₂—);
Q is a bond, a C₁₋₁₀ alkylene group or a C₂₋₁₀ alkenylene group (preferably a bond, —CH₂—, —(CH₂)₂—, —CH═CH—); and
X is a carboxyl group;
   a carbamoyl group;
   a C₁₋₆ alkoxy-carbonyl group;
   a carbamoyl group mono- or di-substituted by a C₁₋₆ alkyl group optionally substituted by 1 to 3 halogen atom(s); or
   a carbamoyl-C₁₋₆ alkyl-carbamoyl group optionally mono- or di-substituted by a C₁₋₆ alkyl group optionally substituted by 1 to 3 halogen atom(s).
      [Compound B]

A compound wherein
R¹ and R² are the same or different and each is
(1) a C₁₋₁₀ alkyl group optionally substituted by 1 to 3 substituent(s) selected from a C₃₋₁₀ cycloalkyl group (preferably cyclopropyl), a C₁₋₆ alkoxy-carbonyl group, a C₁₋₆ alkoxy group and the like;
(2) a C₆₋₁₄ aryl group (preferably phenyl) optionally substituted by 1 to 3 substituent(s) selected from a halogen atom, a carboxyl group, a C₁₋₆ alkoxy-carbonyl group, a carbamoyl group and the like; or
(3) a C₇₋₁₃ aralkyl group (preferably benzyl);
R³ is a C₆₋₁₄ aryl group (the C₆₋₁₄ aryl group is preferably phenyl) optionally substituted by 1 to 3 substituent(s) selected from a C₁₋₆ alkyl group optionally substituted by 1 to 3 halogen atom(s), a halogen atom, a C₁₋₆ alkoxy-carbonyl group, a carboxyl group, a hydroxy group, a C₁₋₆ alkoxy group optionally substituted by 1 to 3 halogen atom(s), and the like;
R⁴ is an amino group optionally mono- or di-substituted by a C₁₋₆ alkyl group (preferably an amino group);
L is a C₁₋₁₀ alkylene group (preferably —CH₂—);
Q is a bond, a C₁₋₁₀ alkylene group or a C₂₋₁₀ alkenylene group (preferably a bond, —CH₂—, —(CH₂)₂—, —CH═CH—); and
X is
(1) a hydrogen atom;
(2) a cyano group;
(3) (3a) a carboxyl group;

(3b) a carbamoyl group;

(3c) a C₁₋₆ alkoxy-carbonyl group optionally substituted by substituent(s) selected from a carboxyl group, a carbamoyl group, a thiocarbamoyl group, a C₁₋₆ alkoxy-carbonyl group and a C₁₋₆ alkyl-carbonyloxy group;

(3d) an aromatic heterocyclic (preferably pyridyl, thiazolyl, oxazolyl, indolyl)-C₁₋₆ alkoxy-carbonyl group optionally substituted by substituent(s) selected from a carboxyl group, a carbamoyl group, a thiocarbamoyl group and a C₁₋₆ alkoxy-carbonyl group;

(3e) a non-aromatic heterocyclic (preferably oxodioxolyl, oxodioxolanyl, oxo-2-benzofuranyl)-C₁₋₆ alkoxy-carbonyl group optionally substituted by a C₁₋₆ alkyl group;

(3f) a C₇₋₁₃ aralkyloxy-carbonyl group optionally substituted by substituent(s) selected from a carboxyl group, carbamoyl group, a thiocarbamoyl group and a C₁₋₆ alkoxy-carbonyl group;

(3g) a carbamoyl group mono- or di-substituted by a C₁₋₆ alkyl group optionally substituted by substituent(s) selected from 1 to 3 halogen atom(s) and a C₁₋₆ alkoxy group;

(3h) a carbamoyl-C₁₋₆ alkyl-carbamoyl group optionally mono- or di-substituted by a C₁₋₆ alkyl group optionally substituted by 1 to 3 halogen atom(s);

(3i) a C₁₋₆ alkoxy-carbonyl-C₁₋₆ alkyl-carbamoyl group optionally substituted by a C₁₋₆ alkyl group;

(3j) a mono- or di-C₃₋₁₀ cycloalkyl-carbamoyl group optionally substituted by a C₁₋₆ alkyl group;

(3k) a C₇₋₁₃ aralkyl-carbamoyl group optionally substituted by substituent(s) selected from a halogen atom, a hydroxy group, a C₁₋₆ alkoxy-carbonyl group and a C₁₋₆ alkyl group;

(3l) an aromatic heterocyclic (preferably pyridyl, thiazolyl, oxazolyl, indolyl)-C₁₋₆ alkyl-carbamoyl group;

(3m) a C₁₋₆ alkylsulfonyl group optionally substituted by substituent(s) selected from a carboxyl group, a carbamoyl group and a C₁₋₆ alkoxy-carbonyl group;

(3n) a C₆₋₁₄ arylsulfonyl group optionally substituted by substituent(s) selected from a C₁₋₆ alkyl group, a carboxyl group, a carbamoyl group, a thiocarbamoyl group, a C₁₋₆ alkoxy-carbonyl group and a C₁₋₆ alkylsulfonyl group;

(3o) a nitrogen-containing heterocyclic (preferably pyrrolidinyl, piperidino, piperazinyl, morpholino)-carbonyl group optionally substituted by substituent(s) selected from a hydroxy group and a C₁₋₆ alkoxy-carbonyl group;

(3p) a C₆₋₁₄ aryl-nitrogen-containing heterocyclic (preferably pyrrolidinyl, piperidino, piperazinyl, morpholino)-carbonyl group optionally substituted by a halogen atom;

(3q) a C₇₋₁₃ aralkyl-nitrogen-containing heterocyclic (preferably pyrrolidinyl, piperidino, piperazinyl, morpholino)-carbonyl group optionally substituted by a halogen atom;

(3r) a non-aromatic heterocyclic (preferably oxodioxolyl, oxodioxolanyl, oxo-2-benzofuranyl)oxy-carbonyl group; or

(3s) a phosphono group optionally mono- or di-substituted by a C₁₋₆ alkyl group;
(4) a C₁₋₆ alkyl-carbonyloxy group;
(5) (5a) a C₁₋₆ alkylthio group optionally substituted by substituent(s) selected from a carboxyl group, a carbamoyl group and a C₁₋₆ alkoxy-carbonyl group;

(5b) a C₆₋₁₄ arylthio group (preferably phenylthio) optionally substituted by substituent(s) selected from a carboxyl group, a C₁₋₆ alkoxy-carbonyl group and a C₁₋₆ alkylthio group; or

(5c) a 5-membered aromatic heterocyclylthio group (preferably thiazolylthio, oxazolylthio, triazolylthio) optionally substituted by a C₁₋₆ alkyl group;
(6) (6a) an amino group;

(6b) a C₁₋₆ alkoxy-carbonyl-C₁₋₁₀ alkylamino group (preferably methoxycarbonylmethylamino, ethoxycarbonylmethylamino, tert-butoxycarbonylmethylamino);

(6c) a carboxy-C₁₋₁₀ alkylamino group;

(6d) a C₇₋₁₃ aralkyloxy-carbonylamino group;

(6e) a carbamoylamino group;

(6f) a mono- or di-C₁₋₆ alkyl-carbamoylamino group;

(6g) a C₁₋₆ alkylsulfonylamino group;

(6h) a C₆₋₁₄ arylsulfonylamino group optionally substituted by a C₁₋₆ alkylsulfonyl group; or

(6i) an aromatic heterocyclic (e.g., pyridyl, thiazolyl, oxazolyl, indolyl)-sulfonylamino group optionally substituted by substituent(s) selected from a C₁₋₆ alkyl group and a mono- or di-(C₁₋₆ alkyl-carbonyl)-amino group; or
(7) tetrazolyl, oxoimidazolidinyl (preferably 2-oxoimidazolidin-1-yl), dioxoimidazolidinyl (preferably 2,4-dioxoimidazolidin-3-yl), oxopiperazinyl (preferably 3-oxopiperazin-1-yl), dioxopiperazinyl (preferably 2,3-dioxopiperazin-1-yl, 2,5-dioxopiperazin-1-yl) or oxodihydrooxadiazolyl (preferably 5-oxo-4,5-dihydro-1,2,4-oxadiazol-3-yl).
   [Compound C]

A compound wherein R⁴ is an amino group, and X is any of the aforementioned (3a)-(3s) in the aforementioned Compound B.

[Compound D]

A compound wherein
R¹, R², R³, R⁴, L and Q are as defined for the aforementioned Compound B, X is
(1) a hydrogen atom;
(2) a cyano group;
(3) (3a) a carboxyl group;

(3b) a carbamoyl group;

(3c) a C₁₋₆ alkoxy-carbonyl group optionally substituted by 1 to 3 substituent(s) selected from a carboxyl group, a carbamoyl group, a thiocarbamoyl group, a C₁₋₆ alkoxy-carbonyl group and a C₁₋₆ alkyl-carbonyloxy group;

(3d) an aromatic heterocyclic (preferably furyl, thienyl, pyridyl, thiazolyl, oxazolyl, pyrazinyl, indolyl)-C₁₋₆ alkoxy-carbonyl group optionally substituted by 1 to 3 substituent(s) selected from a carboxyl group, a carbamoyl group, a thiocarbamoyl group and a C₁₋₆ alkoxy-carbonyl group;

(3e) a non-aromatic heterocyclic (preferably oxodioxolyl, oxodioxolanyl, oxo-2-benzofuranyl)-C₁₋₆ alkoxy-carbonyl group optionally substituted by a C₁₋₆ alkyl group;

(3f) a C₇₋₁₃ aralkyloxy-carbonyl group optionally substituted by 1 to 3 substituent(s) selected from a carboxyl group, a carbamoyl group, a thiocarbamoyl group, a C₁₋₆ alkoxy-carbonyl group, a halogen atom, a cyano group, a nitro group, a C₁₋₆ alkoxy group, a C₁₋₆ alkylsulfonyl group and a C₁₋₆ alkyl group (the C₁₋₆ alkyl group is optionally substituted by 1 to 3 substituent(s) selected from a halogen atom, a carboxyl group, C₁₋₆ alkoxy-carbonyl group and a carbamoyl group);

(3g) a carbamoyl group mono- or di-substituted by a C₁₋₆ alkyl group optionally substituted by 1 to 3 substituent(s) selected from a halogen atom and a C₁₋₆ alkoxy group;

(3h) a carbamoyl-C₁₋₆ alkyl-carbamoyl group optionally mono- or di-substituted by a C₁₋₆ alkyl group optionally substituted by 1 to 3 halogen atom(s);

(3i) a C₁₋₆ alkoxy-carbonyl-C₁₋₆ alkyl-carbamoyl group optionally substituted by a C₁₋₆ alkyl group;

(3j) a mono- or di-C₃₋₁₀ cycloalkyl-carbamoyl group optionally substituted by a C₁₋₆ alkyl group;

(3k) a C₇₋₁₃ aralkyl-carbamoyl group optionally substituted by 1 to 3 substituent(s) selected from a halogen atom, a hydroxy group, a carboxyl group, a C₁₋₆ alkoxy-carbonyl group and a C₁₋₆ alkyl group;

(3l) an aromatic heterocyclic (preferably pyridyl, thienyl, furyl, thiazolyl, oxazolyl, indolyl)-C₁₋₆ alkyl-carbamoyl group optionally substituted by 1 to 3 substituent(s) selected from a carboxyl group, a carbamoyl group and a C₁₋₆ alkoxy-carbonyl group;

(3m) a C₁₋₆ alkylsulfonyl group optionally substituted by 1 to 3 substituent(s) selected from a carboxyl group, a carbamoyl group and a C₁₋₆ alkoxy-carbonyl group;

(3n) a C₆₋₁₄ arylsulfonyl group optionally substituted by 1 to 3 substituent(s) selected from a C₁₋₆ alkyl group, a carboxyl group, a carbamoyl group, a thiocarbamoyl group, a C₁₋₆ alkoxy-carbonyl group and a C₁₋₆ alkylsulfonyl group;

(3o) a nitrogen-containing heterocyclic (preferably pyrrolidinyl, piperidinyl, piperazinyl, morpholino)-carbonyl group optionally substituted by 1 to 3 substituent(s) selected from a hydroxy group, a carboxyl group and a C₁₋₆ alkoxy-carbonyl group;

(3p) a C₆₋₁₄ aryl-nitrogen-containing heterocyclic (preferably pyrrolidinyl, piperidinyl, piperazinyl, morpholino)-carbonyl group optionally substituted by 1 to 3 halogen atom(s);

(3q) a C₇₋₁₃ aralkyl-nitrogen-containing heterocyclic (preferably pyrrolidinyl, piperidinyl, piperazinyl, morpholino)-carbonyl group optionally substituted by 1 to 3 halogen atom(s);

(3r) a non-aromatic heterocyclic (preferably oxodioxolyl, oxodioxolanyl, oxo-2-benzofuranyl)oxy-carbonyl group;

(3s) a phosphono group optionally mono- or di-substituted by a C₁₋₆ alkyl group;

(3t) an aromatic heterocyclic (preferably tetrazoyly)-C₇₋₁₃ aralkyloxy-carbonyl group;

(3u) a C₃₋₁₀ cycloalkyl-C₁₋₆ alkoxy-carbonyl group optionally substituted by 1 to 3 substituent(s) selected from a carboxyl group, a C₁₋₆ alkoxy-carbonyl group and a carbamoyl group;

(3v) a C₆₋₁₄ aryl-carbamoyl group optionally substituted by 1 to 3 substituent(s) selected from an amino group optionally mono- or di-substituted by a C₁₋₆ alkyl group, a carboxyl group, a C₁₋₆ alkoxy-carbonyl group, an aromatic heterocyclic group (preferably tetrazolyl, oxadiazolyl), a non-aromatic heterocyclic group (preferably oxooxadiazolyl) and a carbamoyl group; or

(3w) an aromatic heterocyclic (preferably thienyl, furyl)-carbamoyl group optionally substituted by 1 to 3 substituent(s) selected from a carboxyl group, a C₁₋₆ alkoxy-carbonyl group and a carbamoyl group;
(4) (4a) a C₁₋₆ alkyl-carbonyloxy group;

(4b) a C₁₋₁₀ alkoxy group optionally substituted by 1 to 3 substituent(s) selected from a hydroxy group, a carboxyl group, a carbamoyl group and a C₁₋₆ alkoxy-carbonyl group;

(4c) a C₆₋₁₄ aryloxy group optionally substituted by 1 to 3 substituent(s) selected from a halogen atom, a carboxyl group, a C₁₋₆ alkoxy-carbonyl group, a C₁₋₆ alkylthio group, a carbamoyl group, a C₁₋₆ alkoxy group, a C₁₋₆ alkylsulfonyl group, a C₁₋₆ alkylsulfinyl group and a C₁₋₆ alkyl group (the C₁₋₆ alkyl group is optionally substituted by 1 or 2 substituent(s) selected from a carboxyl group, a C₁₋₆ alkoxy-carbonyl group and a carbamoyl group);

(4d) a 5- or 6-membered aromatic heterocyclyloxy group (preferably thienyloxy, thiazolyloxy, oxazolyloxy, imidazolyloxy, triazolyloxy, pyrazolyloxy, pyridyloxy, pyrimidinyloxy) optionally substituted by 1 to 3 substituent(s) selected from a C₁₋₆ alkyl group (the C₁₋₆ alkyl group is optionally substituted by 1 or 2 substituent(s) selected from a carboxyl group, a C₁₋₆ alkoxy-carbonyl group and a carbamoyl group), a carboxyl group, a C₁₋₆ alkoxy-carbonyl group and a carbamoyl group;

(4e) a fused aromatic heterocyclyloxy group (preferably indolyloxy) optionally substituted by 1 to 3 substituent(s) selected from a carboxyl group, a C₁₋₆ alkoxy-carbonyl group and a carbamoyl group;

(4f) an aromatic heterocyclic (preferably pyridyl)-C₁₋₆ alkoxy group optionally substituted by 1 to 3 substituent(s) selected from a carboxyl group, a C₁₋₆ alkoxy-carbonyl group and a carbamoyl group; or

(4g) an aromatic heterocyclic (preferably tetrazolyl)-C₆₋₁₄ aryloxy group;
(5) (5a) a C₁₋₆ alkylthio group optionally substituted by 1 to 3 substituent(s) selected from a hydroxy group, a carboxyl group, a carbamoyl group and a C₁₋₆ alkoxy-carbonyl group;

(5b) a C₆₋₁₄ arylthio group optionally substituted by 1 to 3 substituent(s) selected from a carboxyl group, a C₁₋₆ alkoxy-carbonyl group, a C₁₋₆ alkylthio group and a carbamoyl group; or

(5c) a 5- or 6-membered aromatic heterocyclylthio group (preferably thienylthio, thiazolylthio, oxazolylthio, imidazolylthio, triazolylthio, pyrazolylthio, pyridylthio, pyrimidinylthio) optionally substituted by 1 to 3 substituent(s) selected from a C₁₋₆ alkyl group, a carboxyl group, a C₁₋₆ alkoxy-carbonyl group and a carbamoyl group;
(6) (6a) an amino group;

(6b) a C₁₋₆ alkoxy-carbonyl-C₁₋₁₀ alkylamino group;

(6c) a carboxy-C₁₋₁₀ alkylamino group;

(6d) a C₇₋₁₃ aralkyloxy-carbonylamino group optionally substituted by 1 to 3 substituent(s) selected from a carboxyl group, a C₁₋₆ alkoxy-carbonyl group and a carbamoyl group;

(6e) a carbamoylamino group;

(6f) a mono- or di-C₁₋₆ alkyl-carbamoylamino group;

(6g) a C₁₋₆ alkylsulfonylamino group;

(6h) a C₆₋₁₄ arylsulfonylamino group optionally substituted by a C₁₋₆ alkylsulfonyl group;

(6i) an aromatic heterocyclic (e.g., pyridyl, thiazolyl, oxazolyl, indolyl)-sulfonylamino group optionally substituted by 1 to 3 substituent(s) selected from a C₁₋₆alkyl group and a mono- or di-(C₁₋₆ alkyl-carbonyl)-amino group;

(6j) a mono- or di-(C₁₋₆ alkyl-carbonyl)-amino group;

(6k) a C₃₋₁₀ cycloalkyl-carbonylamino group;

(6l) a C₆₋₁₄ aryl-carbonylamino group optionally substituted by 1 to 3 substituent(s) selected from a halogen atom, a cyano group, an optionally halogenated C₁₋₆ alkyl group, a C₁₋₆ alkoxy group, a carboxyl group, a C₁₋₆ alkoxy-carbonyl group, an aromatic heterocyclic group (preferably tetrazolyl, oxadiazolyl), a non-aromatic heterocyclic group (preferably oxooxadiazolyl) and a carbamoyl group;

(6m) a C₇₋₁₃ aralkyl-carbonylamino group;

(6n) a C₈₋₁₃ arylalkenyl-carbonylamino group;

(6o) an aromatic heterocyclic (preferably furyl, thienyl, oxazolyl, thiazolyl, isoxazolyl, isothiazolyl, pyrazolyl, pyridyl, pyrazinyl, benzofuryl, benzothienyl, quinoxalinyl)-carbonylamino group optionally substituted by 1 to 3 substituent(s) selected from a C₁₋₆ alkyl group, a C₆₋₁₄ aryl group, a C₇₋₁₃ aralkyl group, a C₁₋₆ alkoxy group, a carboxyl group, a C₁₋₆ alkoxy-carbonyl group and a carbamoyl group;

(6p) a nitrogen-containing heterocyclic (preferably pyrrolidinyl, piperidinyl, piperazinyl, morpholino)-carbonylamino group optionally substituted by 1 to 3 substituent(s) selected from a C₁₋₆ alkyl group (the C₁₋₆ alkyl group is optionally substituted by 1 to 3 substituent(s) selected from a carboxyl group, a C₁₋₆ alkoxy-carbonyl group and a carbamoyl group), a carboxyl group, a C₁₋₆ alkoxy-carbonyl group and a carbamoyl group;

(6q) a C₆₋₁₄ aryl-nitrogen-containing heterocyclic (e.g., pyrrolidinyl, piperidinyl, piperazinyl, morpholino)-carbonylamino group;

(6r) a tetrahydropyranylcarbonylamino group;

(6s) a 4-oxo-4,5,6,7-tetrahydro-1-benzofuranyl-carbonylamino group;

(6t) a C₁₋₆ alkoxy-carbonylamino group optionally substituted by a C₁₋₆ alkoxy-carbonyl group;

(6u) a C₆₋₁₄ aryloxy-carbonylamino group optionally substituted by 1 to 3 substituent(s) selected from a carboxyl group, a C₁₋₆ alkoxy-carbonyl group and a carbamoyl group;

(6v) a C₇₋₁₃ aralkyl-carbamoylamino group; or

(6w) an aromatic heterocyclic (preferably thiazolyl, oxazolyl)-carbamoylamino group optionally substituted by 1 to 3 substituent(s) selected from a carboxyl group, a C₁₋₆ alkoxy-carbonyl group and a carbamoyl group; or
(7) (7a) tetrazolyl;

(7b) oxoimidazolidinyl (preferably 2-oxoimidazolidin-1-yl);

(7c) dioxoimidazolidinyl (preferably 2,4-dioxoimidazolidin-3-yl, 2,4-dioxoimidazolidin-1-yl) optionally substituted by a C₁₋₆ alkyl group optionally substituted by 1 to 3 substituent(s) selected from a carboxyl group and a C₁₋₆ alkoxy-carbonyl group;

(7d) oxopiperazinyl (preferably 3-oxopiperazin-1-yl);

(7e) dioxopiperazinyl (preferably 2,3-dioxopiperazin-1-yl, 2,5-dioxopiperazin-1-yl);

(7f) oxodihydrooxadiazolyl (preferably 5-oxo-4,5-dihydro-1,2,4-oxadiazol-3-yl);

(7g) dioxoisoindolyl;

(7h) oxazolyl optionally substituted by a C₁₋₆ alkoxy-carbonyl group;

(7i) dioxooxazolidinyl (preferably 2,4-dioxooxazolidin-5-yl) or dioxothiazolidinyl (preferably 2,4-dioxothiazolidin-5-yl), each of which is optionally substituted by a C₁₋₆ alkyl group optionally substituted by 1 to 3 substituent(s) selected from a carboxyl group and a C₁₋₆ alkoxy-carbonyl group;

(7j) 4-oxo-2-thioxo-1,3-thiazolidin-5-yl or 4-oxo-2-thioxo-1,3-oxazolidin-5-yl, each of which is optionally substituted by a C₁₋₆ alkyl group optionally substituted by 1 to 3 substituent(s) selected from a carboxyl group and a C₁₋₆ alkoxy-carbonyl group;

(7k) 1,3(2H,5H)-dioxo-tetrahydroimidazo[1,5-a]pyridinyl;

(7l) 1,3(2H,5H)-dioxo-10,10a-dihydroimidazo[1,5-b]isoquinolinyl; or

(7m) a C₆₋₁₄ aryl group optionally substituted by a C₁₋₆ alkoxy-carbonyl group.

[Compound E]

The aforementioned Compound D wherein
R¹ and R² are the same or different and each is a C₁₋₁₀ alkyl group (preferably R¹ is isobutyl or neopentyl;
R² is methyl);
R³ is a C₆₋₁₄ aryl group optionally substituted by a C₁₋₆ alkyl group (R³ is preferably 4-methylphenyl);
R⁴ is an amino group; and
X is the aforementioned (3a), (3c), (3f), (3o), (3v), (4d), (5b), (6l) or (6o) [preferably (3a), (3o), (3v), (4d) or (6o)].
   [Compound F]
5-(Aminomethyl)-2-methyl-4-(4-methylphenyl)-6-neopentylnicotinic acid (Example 22);
5-(aminomethyl)-6-isobutyl-2-methyl-4-(4-methylphenyl)nicotinic acid (Example 40);
methyl 3-{[5-(aminomethyl)-6-isobutyl-2-methyl-4-(4-methylphenyl)pyridin-3-yl]methoxy}-1-methyl-1H-pyrazole-4-carboxylate (Example 305);
{[2-isobutyl-6-methyl-4-(4-methylphenyl)-5-(2-morpholin-4-yl-2-oxoethyl)pyridin-3-yl]methyl}amine (Example 312);
methyl 3-({[5-(aminomethyl)-6-isobutyl-2-methyl-4-(4-methylphenyl)pyridin-3-yl]acetyl}amino)benzoate (Example 336);
N-[5-(aminomethyl)-6-isobutyl-2-methyl-4-(4-methylphenyl)pyridin-3-yl]isoxazole-4-carboxamide (Example 350); or a salt thereof (preferably hydrochloride, trifluoroacetate, fumarate).

As a salt of compound (I), a pharmacologically acceptable salt is preferable. Examples of such salt include salts with inorganic bases, salts with organic bases, salts with inorganic acids, salts with organic acids, salts with basic or acidic amino acids and the like.

Preferable examples of the salt with inorganic base include alkali metal salts such as sodium salt, potassium salt and the like; alkaline earth metal salts such as calcium salt, magnesium salt and the like; aluminum salt; ammonium salt and the like.

Preferable examples of the salt with organic base include a salt with trimethylamine, triethylamine, pyridine, picoline, ethanolamine, diethanolamine, triethanolamine, tromethamine[tris(hydroxymethyl)methylamine], tert-butylamine, cyclohexylamine, benzylamine, dicyclohexylamine, N,N-dibenzylethylenediamine and the like.

Preferable examples of the salt with inorganic acid include a salt with hydrochloric acid, hydrobromic acid, nitric acid, sulfuric acid, phosphoric acid and the like.

Preferable examples of the salt with organic acid include a salt with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like.

Preferable examples of the salt with basic amino acid include a salt with arginine, lysin, ornithine and the like.

Preferable examples of the salt with acidic amino acid include a salt with aspartic acid, glutamic acid and the like.

Of the above-mentioned salts, the salt with inorganic acid and the salt with organic acid are preferable, hydrochloride, trifluoroacetate, fumarate and the like are more preferable.

A prodrug of compound (I) is a compound that converts to compound (I) due to the reaction by enzyme, gastric acid and the like under the physiological conditions in the body; that is, a compound that converts to compound (I) by enzymatic oxidation, reduction, hydrolysis and the like, and a compound that converts to compound (I) by hydrolysis and the like by gastric acid and the like. Examples of a prodrug of compound (I) include a compound wherein an amino group of compound (I) is acylated, alkylated, phosphorylated (e.g., compound where amino group of compound (I) is eicosanoylated, alanylated, pentylaminocarbonylated, (5-methyl-2-oxo-1,3-dioxolen-4-yl)methoxycarbonylated, tetrahydrofuranylated, pyrrolidylmethylated, pivaloyloxymethylated, tert-butylated and the like); a compound wherein a hydroxy group of compound (I) is acylated, alkylated, phosphorylated, borated (e.g., a compound where a hydroxy group of compound (I) is acetylated, palmitoylated, propanoylated, pivaloylated, succinylated, fumarylated, alanylated, dimethylaminomethylcarbonylated and the like); a compound wherein a carboxyl group of compound (I) is esterified or amidated (e.g., a compound where a carboxyl group of compound (I) is ethyl esterified, phenyl esterified, carboxymethyl esterified, dimethylaminomethyl esterified, pivaloyloxymethyl esterified, ethoxycarbonyloxyethyl esterified, phthalidyl esterified, (5-methyl-2-oxo-1,3-dioxolen-4-yl)methyl esterified, cyclohexyloxycarbonylethyl esterified, methylamidated and the like) and the like. These compounds can be produced from compound (I) by a method known per se.

A prodrug of compound (I) may be a compound that converts to compound (I) under physiological conditions as described in Development of Pharmaceutical Products, vol. 7, Molecule Design, 163-198, Hirokawa Shoten (1990).

The compound (I) may be labeled with an isotope (e.g., ³H, ¹⁴C, ³⁵S, ¹²⁵I and the like) and the like.

The compound (I) may be an anhydride or a hydrate.

The compound (I) and a prodrug thereof (hereinafter sometimes to be simply referred to as the compound of the present invention) show low toxicity and can be used as an agent for the prophylaxis or treatment of various diseases to be mentioned later for mammals (e.g., human, mouse, rat, rabbit, dog, cat, cattle, horse, swine, simian and the like) as they are or by admixing with a pharmacologically acceptable carrier and the like to give a pharmaceutical composition.

Here, various organic or inorganic carriers conventionally used as materials for pharmaceutical preparations are used as a pharmacologically acceptable carrier, which are added as excipient, lubricant, binder, disintegrant for solid preparations; and solvent, dissolution aids, suspending agent, isotonicity agent, buffer, soothing agent and the like for liquid preparations. Where necessary, additive for pharmaceutical preparations such as preservative, antioxidant, coloring agent, sweetening agent and the like can be used.

Preferable examples of the excipient include lactose, sucrose, D-mannitol, D-sorbitol, starch, pregelatinized starch, dextrin, crystalline cellulose, low-substituted hydroxypropyl cellulose, sodium carboxymethylcellulose, powdered acacia, dextrin, pullulan, light silicic anhydride, synthetic aluminum silicate, magnesium aluminate metasilicate and the like.

Preferable examples of the lubricant include magnesium stearate, calcium stearate, talc, colloidal silica and the like.

Preferable examples of the binder include pregelatinized starch, saccharose, gelatin, powdered acacia, methylcellulose, carboxymethylcellulose, sodium carboxymethylcellulose, crystalline cellulose, sucrose, D-mannitol, trehalose, dextrin, pullulan, hydroxypropyl cellulose, hydroxypropyl methylcellulose, polyvinylpyrrolidone and the like.

Preferable examples of the disintegrant include lactose, sucrose, starch, carboxymethylcellulose, calcium carboxymethylcellulose, sodium croscarmellose, sodium carboxymethyl starch, light silicic anhydride, low-substituted hydroxypropyl cellulose and the like.

Preferable examples of the solvent include water for injection, physiological brine, Ringer's solution, alcohol, propylene glycol, polyethylene glycol, sesame oil, corn oil, olive oil, cottonseed oil and the like.

Preferable examples of the dissolution aids include polyethylene glycol, propylene glycol, D-mannitol, trehalose, benzyl benzoate, ethanol, trisaminomethane, cholesterol, triethanolamine, sodium carbonate, sodium citrate, sodium salicylate, sodium acetate and the like.

Preferable examples of the suspending agent include surfactants such as stearyltriethanolamine, sodium lauryl sulfate, lauryl aminopropionate, lecithin, benzalkonium chloride, benzethonium chloride, glycerol monostearate and the like; hydrophilic polymers such as polyvinyl alcohol, polyvinylpyrrolidone, sodium carboxymethylcellulose, methylcellulose, hydroxymethylcellulose, hydroxyethylcellulose, hydroxypropyl cellulose and the like; polysorbates, polyoxyethylene hydrogenated castor oil; and the like.

Preferable examples of the isotonicity agent include sodium chloride, glycerol, D-mannitol, D-sorbitol, glucose and the like.

Preferable examples of the buffer include phosphate buffer, acetate buffer, carbonate buffer, citrate buffer and the like.

Preferable examples of the soothing agent include benzyl alcohol and the like.

Preferable examples of the preservative include p-oxybenzoates, chlorobutanol, benzyl alcohol, phenethyl alcohol, dehydroacetic acid, sorbic acid and the like.

Preferable examples of the antioxidant include sulfite, ascorbate and the like.

Preferable examples of the coloring agent include water-soluble edible tar pigments (e.g., foodcolors such as Food Color Red Nos. 2 and 3, Food Color Yellow Nos. 4 and 5, Food Color Blue Nos. 1 and 2 and the like), water insoluble lake pigments (e.g., aluminum salt of the aforementioned water-soluble edible tar pigment and the like), natural pigments (e.g., beta carotene, chlorophil, red iron oxide etc.) and the like.

Preferable examples of the sweetening agent include saccharin sodium, dipotassium glycyrrhizinate, aspartame, stevia and the like.

The dosage form of the aforementioned pharmaceutical composition is, for example, an oral agent such as tablets (inclusive of sublingual tablets and orally disintegrable tablets), capsules (inclusive of soft capsules and micro capsules), granules, powders, troches, syrups, emulsions, suspensions and the like; or a parenteral agent such as injections (e.g., subcutaneous injections, intravenous injections, intramuscular injections, intraperitoneal injections, drip infusions etc.), external agents (e.g., transdermal preparations, ointments etc.), suppositories (e.g., rectal suppositories, vaginal suppositories etc.), pellets, nasal preparations, pulmonary preparations (inhalations), ophthalmic preparations and the like. These may be administered safely via an oral or parenteral route.

These agents may be controlled-release preparations such as rapid-release preparations and sustained-release preparations (e.g., sustained-release microcapsules).

The pharmaceutical composition can be produced according to a method conventionally used in the field of pharmaceutical preparation, such as the method described in Japan Pharmacopoeia and the like. Specific production methods of the pharmaceutical preparation are described in detail in the following.

While the content of the compound of the present invention in the pharmaceutical composition varies depending on the dosage form, dose of the compound of the present invention and the like, it is, for example, about 0.1-100 wt %.

For example, an oral agent is produced by adding, to the active ingredient, excipients (e.g., lactose, sucrose, starch, D-mannitol and the like), disintegrants (e.g., calcium carboxymethylcellulose and the like), binders (e.g., pregelatinized starch, powdered acacia, carboxymethylcellulose, hydroxypropyl cellulose, polyvinylpyrrolidone and the like), lubricants (e.g., talc, magnesium stearate, polyethylene glycol 6000 and the like) and the like, compression-molding the obtained mixture, and where necessary, coating the same using a coating base for masking of taste, enteric property or sustained release according to a method known per se.

Examples of the coating base include a sugar-coating base, a water-soluble film coating base, an enteric film coating base, a sustained release film coating base and the like.

As the sugar-coating base, sucrose may be used, if necessary, along with one or more species selected from talc, precipitated calcium carbonate, gelatin, powdered acacia, pullulan, carnauba wax and the like.

As the water-soluble film coating base, for example, cellulose polymers such as hydroxypropyl cellulose, hydroxypropyl methylcellulose, hydroxyethylcellulose, methylhydroxyethylcellulose and the like; synthetic polymers such as polyvinyl acetal diethylaminoacetate, aminoalkyl methacrylate copolymer E [Eudragit E, trade name, Roehm Pharma], polyvinylpyrrolidone and the like; polysaccharides such as pullulan and the like; and the like are used.

As the enteric film coating base, for example, cellulose polymers such as hydroxypropyl methylcellulose phthalate, hydroxypropyl methylcellulose acetate succinate, carboxymethylethylcellulose, cellulose acetate phthalate and the like; acrylic acid polymers such as methacrylic acid copolymer L [Eudragit L, trademark, Roehm Pharma], methacrylic acid copolymer LD [Eudragit L-30D55, trade name, Roehm Pharma], methacrylic acid copolymer S [Eudragit S, trade name, Roehm Pharma] and the like; natural products such as shellac and the like; and the like are used.

As the sustained release film coating base, for example, cellulose polymers such as ethylcellulose and the like; acrylic acid polymers such as aminoalkyl methacrylate copolymer RS [Eudragit RS, trade name, Roehm Pharma], ethyl acrylate-methyl methacrylate copolymer suspension [Eudragit NE, trade name, Roehm Pharma] and the like, and the like are used.

Two or more kinds of the above-mentioned coating bases may be mixed in an appropriate ratio for use. In addition, a light shielding agent such as titanium oxide, ferric oxide and the like may be used during coating.

An injection is produced by dissolving, suspending or emulsifying an active ingredient in an aqueous solvent (e.g., distilled water, physiological saline, Ringer's solution and the like) or an oily solvent (e.g., vegetable oil such as olive oil, sesame oil, cottonseed oil, corn oil and the like, propylene glycol and the like) and the like, together with a dispersing agent (e.g., polysorbate 80, polyoxyethylene hydrogenated castor oil 60, polyethylene glycol, carboxymethylcellulose, sodium alginate and the like), preservative (e.g., methylparaben, propylparaben, benzyl alcohol, chlorobutanol, phenol and the like), isotonicity agent (e.g., sodium chloride, glycerol, D-mannitol, D-sorbitol, glucose and the like) and the like. In this step, additives such as dissolution aids (e.g., sodium salicylate, sodium acetate and the like), stabilizers (e.g., human serum albumin and the like), soothing agents (e.g., benzyl alcohol and the like) and the like may be used on demand.

The compound of the present invention shows low toxicity (e.g., acute toxicity, chronic toxicity, genetic toxicity, reproductive toxicity, vascular toxicity, carcinogenic), causes fewer side effects and can be used as an agent for the prophylaxis or treatment or diagnosis of various diseases for mammals (e.g., human, cattle, horse, dog, cat, simian, mouse, rat, especially human).

The compound of the present invention has a superior peptidase inhibitory activity and can suppress peptidase-caused degradation of a physiologically active substance such as peptide hormones, cytokines, neurotransmitters and the like.

Examples of the peptide hormones include glucagon-like peptide-1 (GLP-1), glucagon-like peptide-2 (GLP-2), GIP, growth hormone release hormone (GHRH) and the like.

Examples of the cytokines include chemokine such as RANTES and the like.

Examples of the neurotransmitters include neuropeptide Y and the like.

Examples of the peptidases include EC 3.4.11.1 (Leucyl aminopeptidase), EC 3.4.11.2 (Membrane alanine aminopeptidase), EC 3.4.11.3 (Cystinyl aminopeptidase), EC 3.4.11.4 (Tripeptide aminopeptidase), EC 3.4.11.5 (Prolyl aminopeptidase), EC 3.4.11.6 (Aminopeptidase B), EC 3.4.11.7 (Glutamyl aminopeptidase), EC 3.4.11.9 (Xaa-Pro aminopeptidase), EC 3.4.11.10 (Bacterial leucyl aminopeptidase), EC 3.4.11.13 (Clostridial aminopeptidase), EC 3.4.11.14 (Cytosol alanyl aminopeptidase), EC 3.4.11.15 (Lysyl aminopeptidase), EC 3.4.11.16 (Xaa-Trp aminopeptidase), EC 3.4.11.17 (Tryptophanyl aminopeptidase), EC 3.4.11.18 (Methionyl aminopeptidase), EC 3.4.11.19 (D-stereospecific aminopeptidase), EC 3.4.11.20 (Aminopeptidase Ey), EC 3.4.11.21 (Aspartyl aminopeptidase), EC 3.4.11.22 (Aminopeptidase I), EC 3.4.13.3 (Xaa-His dipeptidase), EC 3.4.13.4 (Xaa-Arg dipeptidase), EC 3.4.13.5 (Xaa-methyl-His dipeptidase), EC 3.4.13.7 (Glu-Glu dipeptidase), EC 3.4.13.9 (Xaa-Pro dipeptidase), EC 3.4.13.12 (Met-Xaa dipeptidase), EC 3.4.13.17 (Non-stereospecific dipeptidase), EC 3.4.13.18 (Cytosol nonspecific dipeptidase), EC 3.4.13.19 (Membrane dipeptidase), EC 3.4.13.20 (Beta-Ala-His dipeptidase), EC 3.4.14.1 (Dipeptidyl-peptidase I), EC 3.4.14.2 (Dipeptidyl-peptidase II), EC 3.4.14.4 (Dipeptidyl-peptidase III), EC 3.4.14.5 (Dipeptidyl-peptidase IV), EC 3.4.14.6 (Dipeptidyl-dipeptidase), EC 3.4.14.9 (Tripeptidyl-peptidase I), EC 3.4.14.10 (Tripeptidyl-peptidase II), EC 3.4.14.11 (Xaa-Pro dipeptidyl-peptidase) and the like as classified by International Union of Biochemistry and Molecular Biology. As peptidase, FAPα, DPP8, DPP9 and the like can be also mentioned.

Of these, EC 3.4.14.1, EC 3.4.14.2, EC 3.4.14.4, EC 3.4.14.5, EC 3.4.14.6, EC 3.4.14.9, EC 3.4.14.10 and EC 3.4.14.11 are preferable. Especially preferred is EC 3.4.14.5 (Dipeptidyl-peptidase IV).

The compound of the present invention may concurrently have a glucagon antagonistic action or a CETP inhibitory action in addition to a peptidase inhibitory action. When the compound of the present invention concurrently has these actions, the compound of the present invention is more effective as an agent for the prophylaxis or treatment of diabetes (e.g., type 1 diabetes, type 2 diabetes, gestational diabetes mellitus etc.) and hyperlipidemia (e.g., hypertriglyceridemia, hypercholesteremia, hypoHDLemia, postprandial hyperlipidemia etc.).

The compound of the present invention is useful as an agent for the prophylaxis or treatment of diabetes (e.g., type 1 diabetes, type 2 diabetes, gestational diabetes and the like); an agent for the prophylaxis or treatment of hyperlipidemia (e.g., hypertriglyceridemia, hypercholesterolemia, hypoHDLemia, postprandial hyperlipidemia and the like); an agent for the prophylaxis or treatment of arteriosclerosis; an agent for the prophylaxis or treatment of impaired glucose tolerance [IGT]; an insulin secretagogue; and an agent for preventing progress of impaired glucose tolerance into diabetes.

For diagnostic criteria of diabetes, Japan Diabetes Society reported new diagnostic criteria in 1999.

According to this report, diabetes is a condition showing any of a fasting blood glucose level (glucose concentration of intravenous plasma) of not less than 126 mg/dl, a 75 g oral glucose tolerance test (75 g OGTT) 2 h level (glucose concentration of intravenous plasma) of not less than 200 mg/dl, and a non-fasting blood glucose level (glucose concentration of intravenous plasma) of not less than 200 mg/dl. A condition not falling under the above-mentioned diabetes and different from “a condition showing a fasting blood glucose level (glucose concentration of intravenous plasma) of less than 110 mg/dl or a 75 g oral glucose tolerance test (75 g OGTT) 2 h level (glucose concentration of intravenous plasma) of less than 140 mg/dl” (normal type) is called a “borderline type”.

In addition, ADA (American Diabetes Association) reported new diagnostic criteria of diabetes in 1997 and WHO in 1998.

According to these reports, diabetes is a condition showing a fasting blood glucose level (glucose concentration of intravenous plasma) of not less than 126 mg/dl and a 75 g oral glucose tolerance test 2 h level (glucose concentration of intravenous plasma) of not less than 200 mg/dl.

According to the above-mentioned reports, impaired glucose tolerance is a condition showing a fasting blood glucose level (glucose concentration of intravenous plasma) of less than 126 mg/dl and a 75 g oral glucose tolerance test 2 h level (glucose concentration of intravenous plasma) of not less than 140 mg/dl and less than 200 mg/dl. According to the report of ADA, a condition showing a fasting blood glucose level (glucose concentration of intravenous plasma) of not less than 110 mg/dl and less than 126 mg/dl is called IFG (Impaired Fasting Glucose). According to the report of WHO, among the IFG (Impaired Fasting Glucose), a condition showing a 75 g oral glucose tolerance test 2 h level (glucose concentration of intravenous plasma) of less than 140 mg/dl is called IFG (Impaired Fasting Glycemia).

The compound of the present invention can be also used as an agent for the prophylaxis or treatment of diabetes, borderline type, impaired glucose tolerance, IFG (Impaired Fasting Glucose) and IFG (Impaired Fasting Glycemia), as determined according to the above-mentioned new diagnostic criteria. Moreover, the compound of the present invention can prevent progress of borderline type, impaired glucose tolerance, IFG (Impaired Fasting Glucose) or IFG (Impaired Fasting Glycemia) into diabetes.

The compound of the present invention can be also used as an agent for the prophylaxis or treatment of, for example, diabetic complications [e.g., neuropathy, nephropathy, retinopathy, cataract, macroangiopathy, osteopenia, hyperosmolar diabetic coma, infectious disease (e.g., respiratory infection, urinary tract infection, gastrointestinal infection, dermal soft tissue infections, inferior limb infection and the like), diabetic gangrene, xerostomia, hypacusis, cerebrovascular disorder, peripheral blood circulation disorder and the like], obesity, osteoporosis, cachexia (e.g., cancerous cachexia, tuberculous cachexia, diabetic cachexia, blood disease cachexia, endocrine disease cachexia, infectious disease cachexia or cachexia due to acquired immunodeficiency syndrome), fatty liver, hypertension, polycystic ovary syndrome, kidney disease (e.g., diabetic nephropathy, glomerular nephritis, glomerulosclerosis, nephrotic syndrome, hypertensive nephrosclerosis, end stage kidney disease and the like), muscular dystrophy, myocardial infarction, angina pectoris, cerebrovascular accident (e.g., cerebral infarction, cerebral apoplexy), Alzheimer's disease, Parkinson's syndrome, anxiety, dementia, insulin resistance syndrome, Syndrome X, metabolic syndrome, hyperinsulinemia, hyperinsulinemia-induced sensory disorder, tumor (e.g., leukemia, breast cancer, prostatic cancer, skin cancer and the like), irritable bowel syndrome, acute or chronic diarrhea, inflammatory diseases (e.g., chronic rheumatoid arthritis, spondylitis deformans, osteoarthritis, lumbago, gout, postoperative or traumatic inflammation, tumentia, neuralgia, pharyngolaryngitis, cystitis, hepatitis (inclusive of nonalcoholic steatohepatitis), pneumonia, pancreatitis, enteritis, inflammatory bowel diseases (including inflammatory disease of large intestine), ulcerative colitis, gastric mucosal injury (inclusive of gastric mucosal injury caused by aspirin) and the like), small intestine mucous membrane trauma, malabsorption, testis function disorder, visceral obesity syndrome and the like.

The compound of the present invention can be also used for decreasing visceral fat, suppressing visceral fat accumulation, improving glycometabolism, improving lipid metabolism, suppressing production of oxidized LDL, improving lipoprotein metabolism, improving coronary artery metabolism, prophylaxis and treatment of cardiovascular complications, prophylaxis and treatment of heart failure complications, lowering blood remnant, prophylaxis and treatment of anovulation, prophylaxis and treatment of hypertrichosis, prophylaxis and treatment of hyperandrogenemia, improving pancreatic (β cell) function, regeneration of pancreatic (β cell), promotion of pancreatic (β cell) regeneration, appetite control and the like.

The compound of the present invention can be also used for secondary prophylaxis and prevention of progression of the above-mentioned various diseases (e.g., cardiovascular event such as myocardial infarction and the like).

The compound of the present invention is a glucose dependent insulin secretagogue that selectively promotes insulin secretion in hyperglycemic patients (e.g., patients showing fasting blood glucose level of not less than 126 mg/dl or 75 g oral glucose tolerance test (75 g OGTT) 2 h level of not less than 140 mg/dl and the like). Therefore, the compound of the present invention is useful as a safe agent for the prophylaxis or treatment of diabetes with a low risk of vascular complications, hypoglycemia induction and the like caused by insulin.

The compound of the present invention is also useful as a therapeutic agent for diabetes with sulfonylurea secondary failure_and affords a superior insulin secretion effect and a hypoglycemic effect for diabetic patients for whom sulfonylurea compounds and fast-acting insulin secretagogues fail to provide an insulin secretion effect, and therefore, fail to provide a sufficient hypoglycemic effect.

As the sulfonylurea compound here, a compound having a sulfonylurea skeleton or a derivative thereof, such as tolbutamide, glibenclamide, gliclazide, chlorpropamide, tolazamide, acetohexamide, glyclopyramide, glimepiride, glipizide, glybuzole and the like can be mentioned.

As the fast-acting insulin secretagogue, a compound that promotes insulin secretion from pancreatic β cell in the same manner as a sulfonylurea compound, though it does not have a sulfonylurea skeleton, such as glinide compounds (e.g., repaglinide, senaglinide, nateglide, mitiglinide, a calcium salt hydrate thereof etc.), and the like, can be mentioned.

While the dose of the compound of the present invention varies depending on the administration subject, administration route, target disease, condition and the like, the compound of the present invention as an active ingredient is generally given in a single dose of about 0.01-100 mg/kg body weight, preferably 0.05-30 mg/kg body weight, more preferably 0.1-10 mg/kg body weight, in the case of, for example, oral administration to adult diabetic patients. This dose is desirably given 1 to 3 times a day.

The compound of the present invention can be used in combination with drugs such as a therapeutic agent of diabetes, a therapeutic agent of diabetic complications, an antihyperlipemic agent, an antihypertensive agent, an antiobestic agent, a diuretic, a chemotherapeutic agent, an immunotherapeutic agent, an antithrombotic agent, a therapeutic agent of osteoporosis, an antidementia agent, an agent for improving erectile dysfunction, a therapeutic agent for incontinentia or pollakiuria, a therapeutic agent for dysurea and the like (hereinafter to be referred to as a combination drug). In this case, the timing of administration of the compound of the present invention and a combination drug is not limited. These may be simultaneously administered to an administration subject or administered in a staggered manner. Moreover, the compound of the present invention and a combination drug may be administered as two kinds of preparations each containing an active ingredient, or may be administered as a single preparation containing both active ingredients.

The dose of the combination drug can be determined as appropriate based on the dose clinically employed. The proportion of the compound of the present invention and combination drug can be appropriately determined depending on the administration subject, administration route, target disease, condition, combination and the like. When, for example, the administration subject is human, a combination drug is used in an amount of 0.01-100 parts by weight per 1 part by weight of the compound of the present invention.

As the therapeutic agent for diabetes, insulin preparations (e.g., animal insulin preparations extracted from the pancreas of bovine and pig; human insulin preparations genetically synthesized using Escherichia coli or yeast; zinc insulin; protamine zinc insulin; fragment or derivative of insulin (e.g., INS-1 etc.), oral insulin preparation and the like), insulin sensitizers (e.g., pioglitazone or a salt thereof (preferably hydrochloride), rosiglitazone or a salt thereof (preferably maleate), Reglixane (JTT-501), GI-262570, Netoglitazone (MCC-555), YM-440, DRF-2593, BM-13.1258, KRP-297, R-119702, Rivoglitazone (CS-011), FK-614, compounds described in WO99/58510 (e.g., (E)-4-[4-(5-methyl-2-phenyl-4-oxazolylmethoxy)benzyloxyimino]-4-phenylbutyric acid), compounds described in WO01/38325, Tesaglitazar (AZ-242), Ragaglitazar (NN-622), Muraglitazar (BMS-298585), ONO-5816, BM-13-1258, LM-4156, MBX-102, LY-519818, MX-6054, LY-510929, Balaglitazone (NN-2344), T-131 or a salt thereof, THR-0921 etc.), PPARγ agonist, PPARγ antagonist, PPARγ/α dual agonist, α-glucosidase inhibitors (e.g., voglibose, acarbose, miglitol, emiglitate etc.), biguanides (e.g., phenformin, metformin, buformin or salts thereof (e.g., hydrochloride, fumarate, succinate) etc.), insulin secretagogues (sulfonylurea (e.g., tolbutamide, glibenclamide, gliclazide, chlorpropamide, tolazamide, acetohexamide, glyclopyramide, glimepiride, glipizide, glybuzole etc.), repaglinide, senaglinide, nateglide, mitiglinide or calcium salt hydrate thereof], GPR40 agonist, GLP-1 receptor agonists [e.g., GLP-1, GLP-1MR, NN-2211, AC-2993 (exendin-4), BIM-51077, Aib(8, 35)hGLP-1(7, 37)NH₂, CJC-1131], amylin agonists (e.g., pramlintide etc.), phosphotyrosine phosphatase inhibitors (e.g., sodium vanadate etc.), dipeptidyl peptidase IV inhibitors (e.g., NVP-DPP-278, PT-100, P32/98, LAF-237, P93/01, TS-021, MK-431, BMS-477118 etc.), β3 agonist (e.g., CL-316243, SR-58611-A, UL-TG-307, SB-226552, AJ-9677, BMS-196085, AZ40140 etc.), gluconeogenesis inhibitors (e.g., glycogen phosphorylase inhibitor, glucose-6-phosphatase inhibitor, glucagon antagonist etc.), SGLT (sodium-glucose cotransporter) inhibitors (e.g., T-1095 etc.), 11β-hydroxysteroid dehydrogenase inhibitors (e.g., BVT-3498 etc.), adiponectin or agonist thereof, IKK inhibitors (e.g., AS-2868 etc.), leptin resistance improving drugs, somatostatin receptor agonists (compounds described in WO01/25228, WO03/42204, WO98/44921, WO98/45285, WO99/22735 etc.), glucokinase activators (e.g., Ro-28-1675) and the like can be mentioned.

Examples of the therapeutic agent for diabetic complications include aldose reductase inhibitors (e.g., Tolrestat, Epalrestat, Zenarestat, Zopolrestat, Minalrestat, Fidarestat (SNK-860), CT-112 etc.), neurotrophic factors and increasing drugs thereof (e.g., NGF, NT-3, BDNF, neurotrophin production-secretion promoters described in WO01/14372 (e.g., 4-(4-chlorophenyl)-2-(2-methyl-1-imidazolyl)-5-[3-(2-methylphenoxy)propyl]oxazole etc.) and the like), neuranagenesis stimulators (e.g., Y-128 etc.), PKC inhibitors (e.g., ruboxistaurin mesylate; LY-333531 etc.), AGE inhibitors (e.g., ALT946, pimagedine, pyratoxanthine, N-phenacylthiazolium bromide (ALT766), ALT-711, EXO-226, Pyridorin, Pyridoxamine etc.), reactive oxygen scavengers (e.g., thioctic acid etc.), cerebral vasodilators (e.g., tiapride, mexiletine etc.), somatostatin receptor agonists (BIM23190) and apoptosis signal regulating kinase-1 (ASK-1) inhibitors.

Examples of the antihyperlipemic agent include statin compounds which are cholesterol synthesis inhibitors (e.g., cerivastatin, pravastatin, simvastatin, lovastatin, atorvastatin, fluvastatin, itavastatin, rosuvastatin, pitavastatin and salts thereof (e.g., sodium salt, calcium salt) etc.), squalene synthase inhibitors (e.g., compounds described in WO97/10224, such as N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid etc.), fibrate compounds (e.g., bezafibrate, clofibrate, simfibrate, clinofibrate etc.), ACAT inhibitors (e.g., Avasimibe, Eflucimibe etc.), anion exchange resins (e.g., colestyramine etc.), probucol, nicotinic acid drugs (e.g., nicomol, niceritrol and the like), ethyl icosapentate, plant sterols (e.g., soysterol, γ-oryzanol etc.) and the like.

Examples of the antihypertensive agent include angiotensin converting enzyme inhibitors (e.g., captopril, enalapril, delapril etc.), angiotensin II antagonists (e.g., candesartan cilexetil, losartan, eprosartan, valsartan, telmisartan, irbesartan, tasosartan, 1-[[2′-(2,5-dihydro-5-oxo-4H-1,2,4-oxadiazol-3-yl)biphenyl-4-yl]methyl]-2-ethoxy-1H-benzimidazole-7-carboxylic acid etc.), calcium antagonists (e.g., manidipine, nifedipine, amlodipine, efonidipine, nicardipine etc.), potassium channel openers (e.g., levcromakalim, L-27152, AL 0671, NIP-121 etc.), Clonidine and the like.

Examples of the antiobestic agent include antiobestic agents acting on the central nervous system (e.g., Dexfenfluramine, fenfluramine, phentermine, Sibutramine, amfepramone, dexamphetamine, Mazindol, phenylpropanolamine, clobenzorex; MCH receptor antagonists (e.g., SB-568849; SNAP-7941; compounds encompassed in WO01/82925 and WO01/87834 etc.); neuropeptide Y antagonists (e.g., CP-422935 etc.); cannabinoid receptor antagonists (e.g., SR-141716, SR-147778 etc.); ghrelin antagonist; 11β-hydroxysteroid dehydrogenase inhibitors (e.g., BVT-3498 etc.) and the like), pancreatic lipase inhibitors (e.g., orlistat, ATL-962 etc.), β3 agonists (e.g., CL-316243, SR-58611-A, UL-TG-307, SB-226552, AJ-9677, BMS-196085, AZ40140 etc.), peptidic anorexiants (e.g., leptin, CNTF (Ciliary Neurotropic Factor) etc.), cholecystokinin agonists (e.g., lintitript, FPL-15849 etc.), feeding deterrent (e.g., P-57 etc.) and the like.

Examples of the diuretic include xanthine derivatives (e.g., sodium salicylate and theobromine, calcium salicylate and theobromine etc.), thiazide preparations (e.g., ethiazide, cyclopenthiazide, trichloromethyazide, hydrochlorothiazide, hydroflumethiazide, benzylhydrochlorothiazide, penflutizide, polythiazide, methyclothiazide etc.), antialdosterone preparations (e.g., spironolactone, triamterene etc.), carbonate dehydratase inhibitors (e.g., acetazolamide and the like), chlorobenzenesulfonamide preparations (e.g., chlortalidone, mefruside, indapamide etc.), azosemide, isosorbide, etacrynic acid, piretanide, bumetanide, furosemide and the like.

Examples of the chemotherapeutic agent include alkylation agents (e.g., cyclophosphamide, ifosfamide etc.), metabolic antagonists (e.g., methotrexate, 5-fluorouracil or its derivative, etc.), anti-cancer antibiotics (e.g., mitomycin, adriamycin etc.), plant-derived anti-cancer agents (e.g., vincristin, vindesine, taxol etc.), cisplatin, carboplatin, etoposide and the like. Of these, furtulon and neofurtulon, which are 5-fluorouracil derivatives, and the like are preferable.

Examples of the immunotherapeutic agent include microorganism or bacterial components (e.g., muramyl dipeptide derivative, picibanil etc.), polysaccharides having immunity potentiating activity (e.g., lentinan, sizofiran, krestin etc.), cytokines obtained by genetic engineering techniques (e.g., interferon, interleukin (IL) etc.), colony stimulating factors (e.g., granulocyte colony stimulating factor, erythropoietin etc.) and the like, with preference given to interleukins such as IL-1, IL-2, IL-12 and the like.

Examples of the antithrombotic agent include heparin (e.g., heparin sodium, heparin calcium, dalteparin sodium etc.), warfarin (e.g., warfarin potassium etc.), anti-thrombin drugs (e.g., aragatroban etc.), thrombolytic agents (e.g., urokinase, tisokinase, alteplase, nateplase, monteplase, pamiteplase etc.), platelet aggregation inhibitors (e.g., ticlopidine hydrochloride, cilostazol, ethyl icosapentate, beraprost sodium, sarpogrelate hydrochloride etc.) and the like.

Examples of the therapeutic agent of osteoporosis include alfacalcidol, calcitriol, elcatonin, calcitonin salmon, estriol, ipriflavone, pamidronate disodium, alendronate sodium hydrate, incadronate disodium and the like.

Examples of the antidementia agent include tacrine, donepezil, rivastigmine, galanthamine and the like.

Examples of the agent for improving erectile dysfunction include apomorphine, sildenafil citrate and the like.

Examples of the therapeutic agent for incontinentia or pollakiuria include flavoxate hydrochloride, oxybutynin hydrochloride, propiverine hydrochloride and the like.

Examples of the therapeutic agent for dysurea include acetylcholine esterase inhibitors (e.g., distigmine) and the like can be mentioned.

Furthermore, drugs having a cachexia-improving action established in animal models and clinical situations, such as cyclooxygenase inhibitors (e.g., Indometacin etc.), Progesterone derivatives (e.g., Megesterol acetate), glucosteroid (e.g., dexamethasone etc.), metoclopramide agents, tetrahydrocannabinol agents, fat metabolism improving agents (e.g., eicosapentaenoic acid etc.), growth hormones, IGF-1, or antibodies to a cachexia-induced factor such as TNF-α, LIF, IL-6, Oncostatin M and the like, can be used in combination with the compound of the present invention.

The combination drug is preferably an insulin preparation, an insulin sensitizer, an α-glucosidase inhibitor, a biguanide, an insulin secretagogue (preferably sulfonylurea) and the like.

Two or more of the above-mentioned combination drugs can be used in combination in an appropriate ratio. Preferable combinations in the case of using two or more combination drugs are, for example, as shown in the following.

1) an insulin secretagogue (preferably sulfonylurea) and an α-glucosidase inhibitor;

2) an insulin secretagogue (preferably sulfonylurea) and a biguanide;

3) an insulin secretagogue (preferably sulfonylurea), a biguanide and an (-glucosidase inhibitor;

4) an insulin sensitizer and an α-glucosidase inhibitor;

5) an insulin sensitizer and a biguanide;

6) an insulin sensitizer, a biguanide and an α-glucosidase inhibitor.

When the compound of the present invention is used in combination with a combination drug, the amount thereof can be reduced within a safe range in consideration of counteraction of these agents. Particularly, the dose of an insulin sensitizer, an insulin secretagogue (preferably sulfonylurea) and a biguanide can be reduced as compared with the normal dose. Therefore, an adverse effect, which may be caused by these agents, can be prevented safely. In addition, the dose of the therapeutic agent of diabetic complications, antihyperlipemic agent and antihypertensive agent can be reduced whereby an adverse effect, which may be caused by these agents, can be prevented effectively.

Hereinafter the production methods of the compound of the present invention are explained.

The compound of the present invention can be produced according to a method known per se, such as a method to be described in detail in the following, or an analogous method thereto.

Compound (I-a), which is a compound of the formula (I) wherein L is La—CH₂—, (wherein La is a bond or a divalent chain hydrocarbon group), X is Xa (wherein Xa is a hydrogen atom, a nitro group, an acyl group, a substituted hydroxy group, an optionally substituted thiol group, an optionally substituted amino group or an optionally substituted cyclic group), and R⁴ is an amino group, can be produced according the following Method A or an analogous method thereto.

As the “divalent chain hydrocarbon group” for La, those similar to the “divalent chain hydrocarbon group” exemplarily recited for the aforementioned L can be mentioned. La is preferably a bond or C₁₋₉ alkylene group.

In addition, as the “acyl group”, “substituted hydroxy group”, “optionally substituted thiol group”, “optionally substituted amino group” and “optionally substituted cyclic group”, each for Xa, those exemplarily recited for the aforementioned X can be used.

When Xa is an ethoxycarbonyl group, then Q is preferably a divalent chain hydrocarbon group 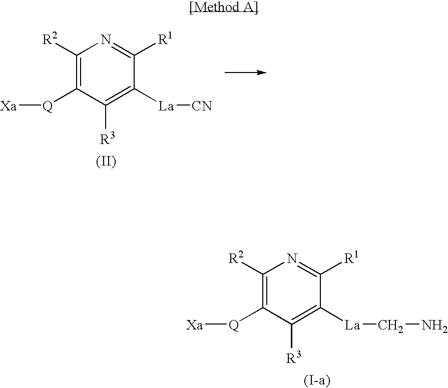
wherein the symbols in the formula are as defined above.

In this method, compound (II) is subjected to a reduction reaction to give compound (I-a).

The reduction reaction is carried out in the presence of a reducing agent, in a solvent that does not adversely influence the reaction, according a conventional method.

As the reducing agent, for example, metal hydrides such as sodium bis(2-methoxyethoxy)aluminum hydride, diisobutylaluminum hydride and the like; metal hydride complexes such as sodium borohydride, sodium cyanoborohydride, lithium aluminum hydride, sodium aluminum hydride and the like; and the like can be mentioned.

The amount of the reducing agent to be used is generally 0.1 to 20 equivalents relative to compound (II).

As the solvent that does not adversely influence the reaction, for example, alcohols such as methanol, ethanol, propanol, 2-propanol, butanol, isobutanol, tert-butanol and the like; aromatic hydrocarbons such as benzene, toluene, xylene and the like; aliphatic hydrocarbons such as hexane, heptane and the like; ethers such as diethyl ether, diisopropyl ether, tert-butylmethyl ether, tetrahydrofuran, dioxane, dimethoxyethane and the like; esters such as methyl acetate, ethyl acetate, n-butyl acetate, tert-butyl acetate and the like; amides such as dimethylformamide, dimethylacetamide, N-methylpyrrolidone and the like, can be used. These solvents may be used in a mixture of two or more kinds thereof mixed at an appropriate ratio.

The reaction temperature is generally -70 to 150° C., preferably −20 to 100° C.

The reaction time is generally 0.1 to 100 hrs, preferably 0.1 to 40 hrs.

The reduction reaction can be also carried out in the presence of a metal catalyst such as palladium-carbon, palladium black, palladium chloride, platinum oxide, platinum black, platinum-palladium, Raney-nickel, Raney-cobalt and the like, and a hydrogen source, in a solvent that does not adversely influence the reaction.

The amount of the metal catalyst to be used is generally, 0.001 to 1000 equivalents, preferably 0.01 to 100 equivalents relative to compound (II).

As the hydrogen source, for example, hydrogen gas, formic acid, formic acid amine salt, phosphinic acid salt, hydrazine and the like can be mentioned.

As the solvent that does not adversely influence the reaction, those used in the aforementioned reduction reaction using the reducing agent can be mentioned.

The reaction temperature and the reaction time are the same as those for the aforementioned reduction reaction using the reducing agent.

This reaction may be carried out in the presence of ammonia (e.g., aqueous ammonia, ammonia-ethanol and the like) where necessary. By the reaction in the presence of ammonia, the side reaction can be suppressed and compound (I-a) can be produced in a high yield.

Compound (I-a) thus obtained can be isolated and purified by a known separation and purification means, such as concentration, concentration under reduced pressure, solvent extraction, crystallization, recrystallization, phase transfer, chromatography and the like.

Compound (II) used as the starting compound in the above-mentioned Method A, can be produced according to a method known per se.

For example, compound (II-a), which is a compound of the formula (II) wherein Q and La are a bond and Xa is an acyl group, can be produced according to the following Method B. 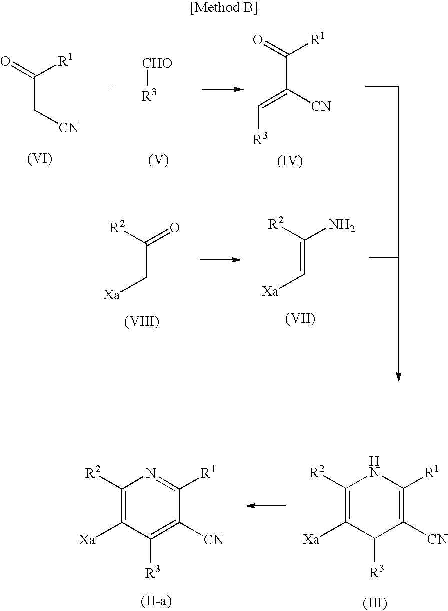
wherein the symbols in the formula are as defined above.

Compound (II-a) can be produced according to a method known per se; for example, by reacting compound (III) and a oxidant such as diluted nitric acid, diammonium cerium nitrate and the like, in a solvent that does not adversely influence the reaction such as 1,4-dioxane, acetone and the like.

Compound (III) can be produced according to a method known per se; for example, from compound (IV) and compound (VII) according to a pyridine synthetic method by Hantzch as described in “Shin Jikken Kagaku Kouza (The Chemical Society of Japan ed.), Vol. 14, Synthesis and Reaction of Organic Compound IV, Maruzen (1978), page 2057, or a method analogous thereto.

Compound (IV) can be produced according to a method known per se, for example, by subjecting compound (VI) and compound (V) to the known Knoevenagel method.

Compound (VII) can be produced according to a method known per se, for example, from compound (VIII) according to the method described in Synthesis (1999), vol. 11, pages 1951-1960; Journal of Chemical Society Perkin Transactions 1, (2002), pages 1663-1671 and the like, or a method analogous thereto.

The aforementioned compound (V), compound (VI) and compound (VIII) can be produced according to a method known per se.

Compound (I-b), which is a compound of the formula (I) wherein R⁴ is an amino group mono- or di-substituted by C₁₋₁₀ alkyl group, can be produced by subjecting compound (I-c), which is a compound of the formula (I) wherein R⁴ is an amino group, to an alkylation reaction.

This reaction is carried out (1) in the presence of base where necessary, using an alkylating agent in a solvent that does not adversely influence the reaction, or (2) in the presence of reducing agent where necessary, using a carbonyl compound in a solvent that does not adversely influence the reaction, according to a method known.

As the alkylating agent here, for example, C₁₋₁₀ alkylhalide, C₁₋₁₀ alkyl sulfonate and the like can be mentioned.

As the carbonyl compound, for example, aldehydes, ketones and the like can be mentioned.

The amount of the alkylating agent and the carbonyl compound to be used are preferably about 1 to about 5 equivalents relative to compound (I-c).

As the base, for example, alkali metal salts such as sodium hydroxide, potassium carbonate and the like; amines such as pyridine, triethylamine and the like; metal hydrides such as sodium hydride and the like; alkali metal alkoxides such as sodium methoxide, potassium t-butoxide and the like, and the like can be mentioned.

The amount of the base to be used is preferably about 1 to about 5 equivalents relative to compound (I-c).

As the reducting agent, for example, metal hydrides such as diisobutylaluminum hydride and the like; metal hydride complexes such as sodium cyanoborohydride and the like; and the like can be mentioned.

The amount of the reducting agent to be used is generally 0.1 to 20 equivalents relative to compound (I-c).

The reaction using the aforementioned carbonyl compound can be also carried out in the presence of a metal catalyst such as palladium-carbon and the like and a hydrogen source, without the reducing agent, in a solvent that does not adversely influence the reaction.

The amount of the metal catalyst to be used is preferably 0.01 to 100 equivalents relative to compound (I-c).

As the hydrogen source, for example, hydrogen gas, formic acid, formic acid amine salt and the like can be mentioned.

As ‘the solvent that does not adversely influence the reaction’ used for the alkylation reaction, for example, aromatic hydrocarbons such as toluene and the like; ethers such as tetrahydrofuran and the like; halogenated hydrocarbons such as chloroform and the like; amides such as N,N-dimethylformamide and the like; sulfoxides such as dimethyl sulfoxide and the like, and the like can be mentioned. These solvents may be used in a mixture thereof mixed at an appropriate ratio.

In the alkylation reaction, the reaction temperature is preferably about −10 to about 100° C.

In the alkylation reaction, the reaction time is generally about 0.5 to about 20 hrs.

Compound (I-b) thus obtained can be isolated and purified by a known separation and purification means, such as concentration, concentration under reduced pressure, solvent extraction, crystallization, recrystallization, phase transfer, chromatography and the like.

Upon producing the compound of the present invention, when the starting compound has amino group, carboxyl group, hydroxy group or carbonyl group as a substituent, a protecting group generally used in peptide chemistry and the like may be introduced into these groups. By removing the protecting group as necessary after the reaction, the objective compound can be obtained.

The amino-protecting group includes, for example, formyl group, C₁₋₆ alkyl-carbonyl group (e.g., acetyl, propionyl and the like), C₁₋₆ alkoxy-carbonyl group (e.g., methoxycarbonyl, ethoxycarbonyl, tert-butoxycarbonyl and the like), benzoyl group, C₇₋₁₃ aralkyl-carbonyl group (e.g., benzylcarbonyl and the like), C₇₋₁₃ aralkyloxy-carbonyl group (e.g., benzyloxycarbonyl, 9-fluorenylmethoxycarbonyl and the like), trityl group, phthaloyl group, N,N-dimethylaminomethylene group, silyl group (e.g., trimethylsilyl, triethylsilyl, dimethylphenylsilyl, tert-butyldimethylsilyl, tert-butyldiethylsilyl and the like), C₂₋₆ alkenyl group (e.g., 1-allyl and the like) and the like. These groups are optionally substituted by 1 to 3 halogen atom(s) (e.g., fluorine, chlorine, bromine, iodine and the like), C₁₋₆ alkoxy group (e.g., methoxy, ethoxy, propoxy and the like), nitro group and the like.

The carboxy-protecting group is, for example, C₁₋₆ alkyl group (e.g., methyl, ethyl, propyl, isopropyl, butyl, tert-butyl and the like), C₇₋₁₃ aralkyl group (e.g., benzyl and the like), phenyl group, trityl group, silyl group (e.g., trimethylsilyl, triethylsilyl, dimethylphenylsilyl, tert-butyldimethylsilyl, tert-butyldiethylsilyl and the like), C₂₋₆ alkenyl group (e.g., 1-allyl and the like) and the like. These groups are optionally substituted by 1 to 3 halogen atom(s) (e.g., fluorine, chlorine, bromine, iodine and the like), C₁₋₆ alkoxy group (e.g., methoxy, ethoxy, propoxy and the like) or nitro group and the like.

The hydroxy-protecting group is, for example, C₁₋₆ alkyl group (e.g., methyl, ethyl, propyl, isopropyl, butyl, tert-butyl and the like), phenyl group, trityl group, C₇₋₁₃ aralkyl group (e.g., benzyl and the like), formyl group, C₁₋₆ alkyl-carbonyl group (e.g., acetyl, propionyl and the like), benzoyl group, C₇₋₁₃ aralkyl-carbonyl group (e.g., benzylcarbonyl and the like), 2-tetrahydropyranyl group, 2-tetrahydrofuranyl group, silyl group (e.g., trimethylsilyl, triethylsilyl, dimethylphenylsilyl, tert-butyldimethylsilyl, tert-butyldiethylsilyl and the like), C₂₋₆ alkenyl group (e.g., 1-allyl and the like) and the like. These groups are optionally substituted by 1 to 3 halogen atom(s) (e.g., fluorine, chlorine, bromine, iodine and the like), C₁₋₆ alkyl group (e.g., methyl, ethyl, propyl and the like), C₁₋₆ alkoxy group (e.g., methoxy, ethoxy, propoxy and the like) or nitro group and the like.

The carbonyl-protecting group is, for example, cyclic acetal (e.g., 1,3-dioxane and the like), non-cyclic acetal (e.g., di-C₁₋₆ alkyl acetal and the like) and the like.

Introduction and removal of these protecting groups can follow a method known per se, for example, a method described in Protective Groups in Organic Synthesis, John Wiley and Sons (1980) and the like. For example, employed is a method using acid, base, UV light, hydrazine, phenyl hydrazine, sodium N-methyldithiocarbamate, tetrabutylammonium fluoride, palladium acetate, trialkylsilyl halide (e.g., trimethylsilyl iodide, trimethylsilyl bromide and the like) and the like, reduction and the like.

When the starting compound can form a salt upon producing the compound of the present invention, the compound in the form of a salt may be used. As such salt, those exemplarily recited above for the salt of compound (I) can be used.

When compound (I) contains an optical isomer, a stereoisomer, a positional isomer or a rotational isomer, these are also encompassed in compound (I), and can be obtained as a single product according to a synthetic method and separation method known per se. For example, when compound (I) has an optical isomer, an optical isomer resolved from this compound is also encompassed in compound (I).

The optical isomer can be produced by a method known per se. To be specific, an optically active synthetic intermediate is used, or the final racemate product is subjected to optical resolution according to a conventional method to give an optical isomer.

The method of optical resolution may be a method known per se, such as a fractional recrystallization method, a chiral column method, a diastereomer method and the like.

1) Fractional Recrystallization Method

A salt of a racemate with an optically active compound (e.g., (+)-mandelic acid, (−)-mandelic acid, (+)-tartaric acid, (−)-tartaric acid, (+)-1-phenethylamine, (−)-1-phenethylamine, cinchonine, (−)-cinchonidine, brucine and the like) is formed, which is separated by a fractional recrystallization method, and a free optical isomer is obtained by a neutralization step where desired.

2) Chiral Column Method

A racemate or a salt thereof is applied to a column for separation of an optical isomer (chiral column) to allow separation. In the case of a liquid chromatography, for example, a mixture of an optical isomer is applied to a chiral column such as ENANTIO-OVM (manufactured by Tosoh Corporation) or CHIRAL series (manufactured by Daicel Chemical Industries, Ltd.) and the like, and developed with water, various buffers (e.g., phosphate buffer) and organic solvents (e.g., ethanol, methanol, isopropanol, acetonitrile, trifluoroacetic acid, diethylamine and the like) solely or in admixture to separate the optical isomer. In the case of a gas chromatography, for example, a chiral column such as CP-Chirasil-DeX CB (manufactured by GL Sciences Inc.) and the like is used to allow separation.

3) Diastereomer Method

A racemic mixture is prepared into a diastereomeric mixture by chemical reaction with an optically active reagent, which is prepared into a single substance by a typical separation means (e.g., fractional recrystallization, chromatography method and the like) and the like, and subjected to a chemical treatment such as hydrolysis and the like to separate an optically active reagent moiety, whereby an optical isomer is obtained. For example, when compound (I) contains hydroxy group or primary or secondary amino group in a molecule, the compound and an optically active organic acid (e.g., MTPA [α-methoxy-α-(trifluoromethyl)phenylacetic acid], (−)-menthoxyacetic acid and the like) and the like are subjected to condensation reaction to give an ester form diastereomer or amide form diastereomer, respectively. When compound (I) has a carboxyl group, this compound and an optically active amine or an optically alcohol reagent are subjected to condensation reaction to give an amide form diastereomer or ester form diastereomer, respectively. The separated diastereomer is converted to an optical isomer of the original compound by acidic hydrolysis or basic hydrolysis reaction.

The compound (I) may be in the form of a crystal.

The crystal of compound (I) (hereinafter sometimes to be referred to as crystal of the present invention) can be produced by crystallization of compound (I) by a crystallization method known per se.

Examples of the crystallization method include crystallization from a solution, crystallization from vapor, crystallization from a molten form and the like.

The “crystallization from a solution” is typically a method including shifting a non-saturated state to supersaturated state by varying factors involved in solubility of compounds (solvent composition, pH, temperature, ionic strength, redox state etc.) or the amount of solvent. To be specific, for example, concentration method, annealing method, reaction method (diffusion method, electrolysis method), hydrothermal growth method, fusing agent method and the like can be mentioned. Examples of the solvent to be used include aromatic hydrocarbons (e.g., benzene, toluene, xylene etc.), halogenated hydrocarbons (e.g., dichloromethane, chloroform etc.), saturated hydrocarbons (e.g., hexane, heptane, cyclohexane etc.), ethers (e.g., diethyl ether, diisopropyl ether, tetrahydrofuran, dioxane etc.), nitriles (e.g., acetonitrile etc.), ketones (e.g., acetone etc.), sulfoxides (e.g., dimethyl sulfoxide etc.), acid amides (e.g., N,N-dimethylformamide and the like), esters (e.g., ethyl acetate etc.), alcohols (e.g., methanol, ethanol, isopropyl alcohol etc.), water and the like. These solvents are used alone or in combination of two or more at a suitable ratio (e.g., 1:1 to 1:100 (volume ratio)).

The “crystallization from vapor” is, for example, vaporization method (sealed tube method, gas stream method), gas phase reaction method, chemical transportation method and the like.

The “crystallization from a molten form” is, for example, normal freezing method (Czockralski method, temperature gradient method, Bridgman method), zone melting method (zone leveling method, floating zone method), special growth method (VLS method, liquid phase epitaxy method) and the like.

Preferable examples of the crystallization method include a method including dissolving compound (I) in a suitable solvent (e.g., alcohols such as methanol, ethanol etc., and the like) at a temperature of 20 to 120° C. and cooling the resulting solution to a temperature not higher than the temperature of dissolution (e.g., 0 to 50° C., preferably 0 to 20° C.) and the like.

The thus-obtained crystals of the present invention can be isolated by, for example, filtration and the like.

In the present specification, the melting point refers to that measured using, for example, micromelting point measuring apparatus (Yanako, MP-500D or Buchi, B-545) or DSC (differential scanning calorimetry) device (SEIKO, EXSTAR6000) and the like.

In general, melting points vary depending on measurement apparatuses, measurement conditions and the like. The crystal in the present specification may show a different melting point described in the present specification, as long as it is within general error range.

The crystal of the present invention is superior in physicochemical properties (e.g., melting point, solubility, stability etc.) and biological properties (e.g., pharmacokinetics (absorption, distribution, metabolism, excretion), efficacy expression etc.), and is extremely useful as a pharmaceutical agent.

### EXAMPLES

The present invention is explained in more detail by the following Examples, Experimental Examples and Formulation Examples. These do not limit the present invention and the present invention can be modified within the range that does not deviate from the scope of the invention.

Abbreviations in the Examples have the following meanings:
s: singlet, d: doublet, t: triplet, q: quartet, m: multiplet, brs: broad singlet, J: coupling constant, 4-Me-Phenyl: 4-methylphenyl, 4-F-Phenyl: 4-fluorophenyl, 2,6-di-F-Phenyl: 2,6-difluorophenyl.

In the Examples, room temperature means the temperature of 1 to 30° C., and % means percent by weight, unless mentioned otherwise.

### Example 1

### methyl 5-(aminomethyl)-6-isobutyl-2-methyl-4-(4-methylphenyl)nicotinate

1) A suspension of sodium hydride (60% in oil, 8.0 g, 0.2 mol) in tetrahydrofuran (80 mL) was heated under reflux with stirring vigorously. A mixture of methyl isovalerate (11.6 g, 0.1 mol), acetonirtile (10.5 mL, 0.2 mol) and tetrahydrofuran (25 mL) was added dropwise to the obtained suspension over 30 min., and the mixture was heated under reflux for 5 hrs. The reaction mixture was allowed to cool to room temperature, and 2-propanol (5 mL) was added thereto. The mixture was stirred at room temperature for 30 min. The reaction mixture was concentrated under reduced pressure, and the residue was dissolved in water (100 mL) and washed successively with hexane and a mixed solution of hexane-diethyl ether. The aqueous layer was acidified with concentrated hydrochloric acid and extracted with diethyl ether. The extract was washed with water and dried over anhydrous magnesium sulfate. The solvent was evaporated under reduced pressure to give 5-methyl-3-oxohexanenitrile (12.6 g, yield 100%) as a yellow oil. The obtained yellow oil was used in the next step without further purification.

¹H-NMR (CDCl₃) δ:0.96 (6H, d, J=6.6 Hz), 2.05-2.30 (1H, m), 2.50 (2H, d, J=7.0 Hz), 3.43 (2H, s).

2) A mixture of 5-methyl-3-oxohexanenitrile (5.0 g, 40 mmol), p-tolualdehyde (4.8 g, 40 mmol), piperidine (0.34 g, 4.0 mmol), acetic acid (0.48 g, 8.0 mmol) and toluene (200 mL) was heated under reflux for 12 hrs. using a Dean-Stark trap. The reaction mixture was allowed to cool to room temperature, washed with saturated brine and dried over anhydrous magnesium sulfate. The solvent was evaporated under reduced pressure and the obtained residue was dissolved in methanol (50 mL). Methyl 3-aminocrotonate (4.6 g, 40 mmol) was added thereto and the mixture was heated under reflux for 6 hrs. The reaction mixture was concentrated under reduced pressure, and the residue was purified by silica gel column chromatography to give methyl 5-cyano-6-isobutyl-2-methyl-4-(4-methylphenyl)-1,4-dihydropyridine-3-carboxylate (7.45 g, yield 57%) as colorless crystals.

¹H-NMR (CDCl₃) δ:0.93 (3H, d, J=6.6 Hz), 0.98 (3H, d, J=6.6 Hz), 1.80-2.00 (1H, m), 2.10-2.35 (2H, m), 2.30 (3H, s), 2.36 (3H, s), 3.58 (3H, s), 4.57 (1H, s), 5.68 (1H, brs), 7.00-7.20 (4H, m).

3) Methyl 5-cyano-6-isobutyl-2-methyl-4-(4-methylphenyl)-1,4-dihydropyridine-3-carboxylate (7.3 g, 22.5 mmol) was dissolved in 1,4-dioxane (20 mL), and 2N nitric acid (100 mL) was added thereto and the mixture was stirred at 70° C. for 1 hr. While stirring in an ice bath, ethyl acetate (100 mL) and 2N aqueous sodium hydroxide solution (100 mL) were added thereto. The aqueous layer was separated and extracted with ethyl acetate. The organic layer and the extract were combined, and the mixture was washed with saturated brine and dried over anhydrous magnesium sulfate. The solvent was evaporated under reduced pressure and the residue was purified by silica gel column chromatography to give methyl 5-cyano-6-isobutyl-2-methyl-4-(4-methylphenyl)nicotinate (5.94 g, yield 82%) as a white powder.

¹H-NMR (CDCl₃) δ:1.01 (6H, d, J=6.6 Hz), 2.20-2.35 (1H, m), 2.41 (3H, s), 2.63 (3H, s), 2.95 (2H, d, J=7.4 Hz), 3.60 (3H, s), 7.20-7.30 (4H, m).

4) A mixture of methyl 5-cyano-6-isobutyl-2-methyl-4-(4-methylphenyl)nicotinate (1.00 g, 3.10 mmol), Raney-nickel (4 mL), 25% aqueous ammonia (6 mL), tetrahydrofuran (15 mL), methanol (45 mL) was stirred in a sealed tube under 0.5 MPa hydrogen atmosphere at room temperature for 6 hrs. The reaction mixture was filtered and the filtrate was concentrated under reduced pressure. The residue was partitioned between ethyl acetate and 10% aqueous potassium carbonate solution. The organic layer was washed with saturated brine and dried over anhydrous magnesium sulfate. The solvent was evaporated under reduced pressure and the residue was purified by silica gel column chromatography to give methyl 5-(aminomethyl)-6-isobutyl-2-methyl-4-(4-methylphenyl)nicotinate (0.97 g, yield 95%) as yellow crystals.

¹H-NMR (CDCl₃) δ:0.98 (6H, d, J=6.6 Hz), 1.39 (2H, brs), 2.15-2.30 (1H, m), 2.39 (3H, s), 2.53 (3H, s), 2.80 (2H, d, J=7.2 Hz), 3.50 (3H, s), 3.66 (2H, s), 7.11 (2H, d, J=8.0 Hz), 7.21 (2H, d, J=8.0 Hz).

melting point: 56-57° C.

### Example 2

### 5-(aminomethyl)-6-isobutyl-2-methyl-4-(4-methylphenyl)nicotinic acid dihydrochloride

1) To a solution of methyl 5-(aminomethyl)-6-isobutyl-2-methyl-4-(4-methylphenyl)nicotinate (0.90 g, 2.76 mmol) in tetrahydrofuran (25 mL) was added di-tert-butyl dicarbonate (0.76 mL, 3.31 mmol), and the mixture was stirred at room temperature for 12 hrs. The reaction mixture was concentrated under reduced pressure, and the residue was purified by silica gel column chromatography to give methyl 5-{[(tert-butoxycarbonyl)amino]methyl}-6-isobutyl-2-methyl-4-(4-methylphenyl)nicotinate (1.16 g, yield 98%) as a white powder.

¹H-NMR (CDCl₃) δ:0.97 (6H, d, J=6.8 Hz), 1.39 (9H, s), 2.10-2.30 (1H, m), 2.39 (3H, s), 2.54 (3H, s), 2.78 (2H, d, J=7.2 Hz), 3.50 (3H, s), 4.15 (2H, d, J=4.9 Hz), 4.24 (1H, t, J=4.9 Hz), 7.06 (2H, d, J=7.9 Hz), 7.20 (2H, d, J=7.9 Hz).

2) To a solution of methyl 5-{[(tert-butoxycarbonyl)amino]methyl}-6-isobutyl-2-methyl-4-(4-methylphenyl)nicotinate (1.0 g, 2.34 mmol) in methanol (30 mL) was added 1N aqueous sodium hydroxide solution (10 mL), and the mixture was heated under reflux for 3 days. The reaction mixture was allowed to cool to room temperature, acidified with 0.5N hydrochloric acid and extracted with ethyl acetate. The extract was washed with saturated brine and dried over anhydrous magnesium sulfate. The solvent was evaporated under reduced pressure and the residue was crystallized from water-methanol to give 5-{[(tert-butoxycarbonyl)amino]methyl}-6-isobutyl-2-methyl-4-(4-methylphenyl)nicotinic acid (0.58 g, yield 60%) as a white powder.

¹H-NMR (CDCl₃) δ:0.87 (6H, d, J=6.4 Hz), 1.39 (9H, s), 1.95-2.10 (1H, m), 2.38 (3H, s), 2.67 (3H, s), 2.75 (2H, d, J=7.2 Hz), 4.13 (2H, d, J=4.7 Hz), 4.30 (1H, t, J=4.7 Hz), 7.15 (2H, d, J=7.9 Hz), 7.22 (2H, d, J=7.9 Hz).

3) To a solution of 5-{[(tert-butoxycarbonyl)amino]methyl}-6-isobutyl-2-methyl-4-(4-methylphenyl)nicotinic acid (0.20 g, 0.48 mmol) in 1,4-dioxane (4 mL) was added 4N hydrogen chloride 1,4-dioxane solution (4 mL, 16 mmol), and the mixture was stirred at room temperature for 2 hrs. The reaction mixture was concentrated under reduced pressure, and the obtained white solid was washed with diisopropyl ether to give 5-(aminomethyl)-6-isobutyl-2-methyl-4-(4-methylphenyl)nicotinic acid dihydrochloride(0.18 g, yield 95%) as a white powder.

¹H-NMR (DMSO-d₆) δ:0.98 (6H, d, J=6.6 Hz), 2.05-2.30 (1H, m), 2.38 (3H, s), 2.65 (3H, s), 3.02 (2H, s), 3.83 (2H, d, J=5.5 Hz), 7.26 (2H, d, J=8.2 Hz), 7.32 (2H, d, J=8.2 Hz), 8.45 (3H, brs).

### Example 3

### 5-(aminomethyl)-6-isobutyl-2-methyl-4-(4-methylphenyl)nicotinamide dihydrochloride

1) A mixture of 5-{[(tert-butoxycarbonyl)amino]methyl}-6-isobutyl-2-methyl-4-(4-methylphenyl)nicotinic acid (0.11 g, 0.27 mmol), 1-hydroxy-1H-benzotriazole ammonium salt (0.10 g, 0.65 mmol), 1-ethyl-3-(3-dimethylaminopropyl)carbodiimide hydrochloride (0.13 g, 0.65 mmol) and N,N-dimethylformamide (10 mL) was stirred at room temperature for 2.5 days. The reaction mixture was partitioned between ethyl acetate (100 mL) and 0.1 M aqueous citric acid solution (50 mL). The organic layer and an extract obtained by extracting the aqueous layer with ethyl acetate were combined, and the mixture was washed successively with saturated aqueous sodium hydrogen carbonate and saturated brine and dried over anhydrous magnesium sulfate. The solvent was evaporated under reduced pressure and the residue was purified by silica gel column chromatography to give tert-butyl{[5-(aminocarbonyl)-2-isobutyl-6-methyl-4-(4-methylphenyl)pyridin-3-yl]methyl}carbamate (0.090 g, yield 82%) as a white powder.

¹H-NMR (CDCl₃) δ:0.97 (6H, d, J=6.6 Hz), 1.39 (9H, s), 2.10-2.30 (1H, m), 2.39 (3H, s), 2.61 (3H, s), 2.78 (2H, d, J=7.4 Hz), 4.14 (2H, d, J=4.7 Hz), 4.15-4.30 (1H, m), 5.22 (1H, brs), 5.41 (1H, brs), 7.11 (2H, d, J=7.9 Hz), 7.23 (2H, d, J=7.9 Hz).

2) 5-(Aminomethyl)-6-isobutyl-2-methyl-4-(4-methylphenyl)nicotinamide dihydrochloride (0.050 g, yield 82%) was obtained as a white powder from tert-butyl{[5-(aminocarbonyl)-2-isobutyl-6-methyl-4-(4-methylphenyl)pyridin-3-yl]methyl}carbamate (0.065 g, 0.16 mmol) according to a method similar to the method of Example 2-3).

¹H-NMR (DMSO-d₆) δ:0.98 (6H, d, J=6.6 Hz), 2.05-2.30 (1H, m), 2.37 (3H, s), 2.66 (3H, s), 3.02 (2H, s), 3.82 (2H, d, J=4.9 Hz), 7.20-7.35 (4H, m), 7.54 (1H, brs), 7.84 (1H, brs), 8.32 (3H, brs).

### Example 4

### 5-(aminomethyl)-N-(3-amino-3-oxopropyl)-6-isobutyl-2-methyl-4-(4-methylphenyl)nicotinamide dihydrochloride

1) A mixture of 5-{[(tert-butoxycarbonyl)amino]methyl}-6-isobutyl-2-methyl-4-(4-methylphenyl)nicotinic acid (0.12 g, 0.29 mmol), β-alaninamide hydrochloride (0.055 g, 0.44 mmol), 1-hydroxy-1H-benzotriazole (0.059 g, 0.44 mmol), 1-ethyl-3-(3-dimethylaminopropyl)carbodiimide hydrochloride (0.084 g, 0.44 mmol), triethylamine (0.061 mL, 0.44 mmol) and N,N-dimethylformamide (5 mL) was stirred at room temperature for 14 hrs. The reaction mixture was partitioned between ethyl acetate-tetrahydrofuran (1:1, 100 mL) and 0.1 M aqueous citric acid solution (100 mL). The organic layer and an extract obtained by extracting the aqueous layer with ethyl acetate were combined, and the mixture was washed successively with saturated aqueous sodium hydrogen carbonate and saturated brine and dried over anhydrous magnesium sulfate. The solvent was evaporated under reduced pressure and the residue was purified by silica gel column chromatography to give tert-butyl{[5-[(3-amino-3-oxopropyl)amino]carbonyl-2-isobutyl-6-methyl-4-(4-methylphenyl)pyridin-3-yl]methyl}carbamate (0.075 g, yield 54%) as a white powder.

¹H-NMR (CDCl₃) δ:0.97 (6H, d, J=6.8 Hz), 1.38 (9H, s), 1.98 (2H, t, J=6.0 Hz), 2.10-2.25 (1H, m), 2.38 (3H, s), 2.55 (3H, s), 2.76 (2H, d, J=7.2 Hz), 3.36 (2H, q, J=6.0 Hz), 4.11 (2H, d, J=5.5 Hz), 4.23 (1H, brs), 5.23 (1H, brs), 5.38 (1H, brs), 6.22 (1H, t, J=5.5 Hz), 7.09 (2H, d, J=8.1 Hz), 7.19 (2H, d, J=8.1 Hz).

2) 5-(Aminomethyl)-N-(3-amino-3-oxopropyl)-6-isobutyl-2-methyl-4-(4-methylphenyl)nicotinamide dihydrochloride (0.048 g, 99%) was obtained as a white powder from tert-butyl{[5-[(3-amino-3-oxopropyl)amino]carbonyl-2-isobutyl-6-methyl-4-(4-methylphenyl)pyridin-3-yl]methyl}carbamate (0.050 g, 0.10 mmol) according to a method similar to the method of Example 2-3).

¹H-NMR (DMSO-d₆) δ:0.97 (6H, d, J=6.6 Hz), 1.98 (2H, t, J=6.7 Hz), 2.10-2.25 (1H, m), 2.37 (3H, s), 2.57 (3H, s), 2.96 (2H, brs), 3.09 (2H, q, J=6.7 Hz), 3.82 (2H, d, J=5.3 Hz), 6.82 (1H, brs), 7.21 (2H, d, J=8.0 Hz), 7.27 (2H, d, J=8.0 Hz), 7.28 (1H, brs), 8.24 (3H, brs), 8.36 (1H, brs).

### Example 5

### [5-(aminomethyl)-6-isobutyl-2-methyl-4-(4-methylphenyl)pyridin-3-yl]acetonitrile

1) A suspension of methyl 5-{[(tert-butoxycarbonyl)amino]methyl}-6-isobutyl-2-methyl-4-(4-methylphenyl)nicotinate (3.4 g, 7.9 mmol) in toluene (80 mL) was cooled to −78° C., and 0.95 M diisobutylaluminum hydride toluene solution (33 mL, 32 mmol) was added dropwise thereto over 15 min. After stirring at −78° C. for 1.5 hrs., the mixture was allowed to warm to 0° C., and further stirred for 30 min. Methanol (1 mL) and sodium sulfate 10 hydrate (10.2 g, 32 mmol) were added successively to the reaction mixture, and the mixture was stirred at room temperature for 1 hr. The insoluble material was filtered off, and the filtrate was concentrated under reduced pressure. The residue was purified by silica gel column chromatography to give tert-butyl{[5-(hydroxymethyl)-2-isobutyl-6-methyl-4-(4-methylphenyl)pyridin-3-yl]methyl}carbamate (1.9 g, yield 60%) as an oil.

¹H-NMR (CDCl₃) δ:0.97 (6H, d, J=6.6 Hz), 1.32 (9H, s), 2.13-2.25 (1H, m), 2.42 (3H, s), 2.68 (3H, s), 2.75 (2H, d, J=7.4 Hz), 4.05 (2H, d, J=4.7 Hz), 4.19 (1H, brs), 4.36 (2H, d, J=5.7 Hz), 7.05 (2H, d, J=7.9 Hz), 7.24-7.26 (2H, m).

2) A mixture of tert-butyl{[5-(hydroxymethyl)-2-isobutyl-6-methyl-4-(4-methylphenyl)pyridin-3-yl]methyl}carbamate (0.50 g, 1.3 mmol), triethylamine (0.35 mL, 2.5 mmol) and tetrahydrofuran (10 mL) was cooled to 0° C., and methanesulfonyl chloride (0.22 g, 1.9 mmol) was added dropwise thereto. After stirring at room temperature for 30 min, the reaction mixture was poured into saturated aqueous sodium hydrogen carbonate, and the mixture was extracted with ethyl acetate. The extract was dried over anhydrous magnesium sulfate and the solvent was evaporated under reduced pressure. The residue was dissolved in dimethyl sulfoxide (5 mL), and potassium cyanide (0.41 g, 6.3 mmol) was added thereto. The mixture was stirred at 60° C. for 30 min. Ethyl acetate was added to the reaction mixture, and the mixture was washed successively with water and saturated brine and dried over anhydrous magnesium sulfate. The solvent was evaporated under reduced pressure and the residue was purified by silica gel column chromatography to give tert-butyl{[5-(cyanomethyl)-2-isobutyl-6-methyl-4-(4-methylphenyl)pyridin-3-yl]methyl}carbamate (0.36 g, yield 72%) as an oil.

¹H-NMR (CDCl₃) δ:0.97 (6H, d, J=6.8 Hz), 1.38 (9H, s), 2.16-2.25 (1H, m), 2.43 (3H, s), 2.66 (3H, s), 2.77 (2H, d, J=7.2 Hz), 3.31 (2H, s), 4.07 (2H, d, J=4.7 Hz), 7.04 (2H, d, J=8.0 Hz), 7.31 (2H, d, J=8.0 Hz).

3) Trifluoroacetic acid (5 mL) was added to tert-butyl{[5-(cyanomethyl)-2-isobutyl-6-methyl-4-(4-methylphenyl)pyridin-3-yl]methyl}carbamate (0.11 g, 0.27 mmol), and the mixture was stirred at room temperature for 15 min. The reaction mixture was poured into saturated aqueous sodium hydrogen carbonate, and the mixture was extracted with ethyl acetate-tetrahydrofuran. The extract was dried over anhydrous magnesium sulfate, and the solvent was evaporated under reduced pressure. The residue was purified by silica gel column chromatography to give [5-(aminomethyl)-6-isobutyl-2-methyl-4-(4-methylphenyl)pyridin-3-yl]acetonitrile (0.084 g, yield 99%) as an oil.

¹H-NMR (CDCl₃) δ:0.99 (6H, d, J=6.6 Hz), 2.11-2.22 (1H, m), 2.45 (3H, s), 2.66 (3H, s), 2.80 (2H, d, J=7.2 Hz), 3.47 (2H, s), 3.74 (2H, brs), 7.17 (2H, d, J=7.8 Hz), 7.42 (2H, d, J=7.8 Hz).

### Example 6

### 2-[5-(aminomethyl)-6-isobutyl-2-methyl-4-(4-methylphenyl)pyridin-3-yl]acetamide dihydrochloride

1) To a solution of tert-butyl{[5-(cyanomethyl)-2-isobutyl-6-methyl-4-(4-methylphenyl)pyridin-3-yl]methyl}carbamate (0.90 g, 2.2 mmol) in ethanol (20 mL) was added 2N aqueous sodium hydroxide solution (5.5 mL, 11 mmol), and the mixture was heated under reflux for 2 hrs. 6N Hydrochloric acid was added to acidify the reaction mixture, and the mixture was extracted with ethyl acetate. The extract was washed with saturated brine and dried over anhydrous magnesium sulfate. The solvent was evaporated under reduced pressure to give tert-butyl{[5-(2-amino-2-oxoethyl)-2-isobutyl-6-methyl-4-(4-methylphenyl)pyridin-3-yl]methyl}carbamate (0.25 g, yield 27%) as a colorless solid.

2) Trifluoroacetic acid (5 mL) was added to tert-butyl{[5-(2-amino-2-oxoethyl)-2-isobutyl-6-methyl-4-(4-methylphenyl)pyridin-3-yl]methyl}carbamate (0.25 g, 0.59 mmol), and the mixture was stirred at room temperature for 20 min. The reaction mixture was poured into saturated aqueous sodium hydrogen carbonate, and the mixture was extracted with ethyl acetate-tetrahydrofuran. The extract was dried over anhydrous magnesium sulfate, and the solvent was evaporated under reduced pressure. 4N Hydrogen chloride 1,4-dioxane solution (4 mL, 16 mmol) was added to the residue, and the solvent was evaporated under reduced pressure. The residue was washed with diisopropyl ether to give 2-[5-(aminomethyl)-6-isobutyl-2-methyl-4-(4-methylphenyl)pyridin-3-yl]acetamide dihydrochloride (0.19 g, yield 81%) as a white powder.

¹H-NMR (CD₃OD) δ:1.09-1.13 (6H, m), 2.09-2.22 (1H, m), 2.46 (3H, s), 2.77-2.80 (3H, m), 3.00-3.09 (2H, m), 3.51-3.55 (2H, m), 4.08 (2H, brs), 7.15-7.22 (2H, m), 7.47 (2H, d, J=8.1 Hz).

### Example 7

### methyl[5-(aminomethyl)-6-isobutyl-2-methyl-4-(4-methylphenyl)pyridin-3-yl]acetate dihydrochloride

1) To a solution of tert-butyl{[5-(cyanomethyl)-2-isobutyl-6-methyl-4-(4-methylphenyl)pyridin-3-yl]methyl}carbamate (0.90 g, 2.2 mmol) in ethanol (20 mL) was added 2N aqueous sodium hydroxide solution (5.5 mL, 11 mmol), and the mixture was heated under reflux for 1.5 days. 6N Hydrochloric acid was added to acidify the reaction mixture, and the mixture was extracted with ethyl acetate. The extract was washed with saturated brine and dried over anhydrous magnesium sulfate. The solvent was evaporated under reduced pressure and the residue was dissolved in N,N-dimethylformamide (5 mL). Methyl iodide (0.65 g, 4.4 mmol) and potassium carbonate (0.61 g, 4.4 mmol) were added thereto, and the mixture was stirred at room temperature for 1 hr. Ethyl acetate was added to the reaction mixture, and the mixture was washed successively with water and saturated brine and dried over anhydrous magnesium sulfate. The solvent was evaporated under reduced pressure and the residue was purified by silica gel column chromatography to give methyl[5-{[(tert-butoxycarbonyl)amino]methyl}-6-isobutyl-2-methyl-4-(4-methylphenyl)pyridin-3-yl]acetate (0.097 g, yield 10%) as an oil.

¹H-NMR (CDCl₃) δ:0.97 (6H, d, J=6.6 Hz), 1.38 (9H, s), 2.13-2.28 (1H, m), 2.40 (3H, s), 2.49 (3H, s), 2.75 (2H, d, J=7.4 Hz), 3.36 (2H, s), 3.61 (3H, s), 4.04-4.05 (2H, m), 4.27 (1H, brs), 6.98 (2H, d, J=7.8 Hz), 7.23 (2H, d, J=7.8 Hz).

2) Methyl[5-(aminomethyl)-6-isobutyl-2-methyl-4-(4-methylphenyl)pyridin-3-yl]acetate dihydrochloride (0.069 g, yield 76%) was obtained as a white powder from methyl[5-{[(tert-butoxycarbonyl)amino]methyl}-6-isobutyl-2-methyl-4-(4-methylphenyl)pyridin-3-yl]acetate (0.097 g, 0.22 mmol) according to a method similar to the method of Example 2-3).

¹H-NMR (CD₃OD) δ:1.09-1.13 (6H, m), 2.12-2.26 (1H, m), 2.47 (3H, s), 2.84 (3H, s), 3.12 (2H, d, J=7.4 Hz), 3.29-3.31 (2H, m), 3.63 (3H, s), 4.08 (2H, s), 7.19 (2H, d, J=7.7 Hz), 7.48 (2H, d, J=7.7 Hz).

### Example 8

### ethyl(2E)-3-[5-(aminomethyl)-6-isobutyl-2-methyl-4-(4-methylphenyl)pyridin-3-yl]acrylate

1) To a solution of tert-butyl{[5-(hydroxymethyl)-2-isobutyl-6-methyl-4-(4-methylphenyl)pyridin-3-yl]methyl}carbamate (1.95 g, 4.9 mmol) in tetrahydrofuran (50 mL) was added manganese dioxide (4.9 g, 56 mmol), and the mixture was stirred at room temperature for 19 hrs. The reaction mixture was filtered and the filtrate was concentrated under reduced pressure. The residue was purified by silica gel column chromatography to give tert-butyl{[5-formyl-2-isobutyl-6-methyl-4-(4-methylphenyl)pyridin-3-yl]methyl}carbamate (1.25 g, yield 65%) as a yellow solid.

¹H-NMR (CDCl₃) δ:0.98 (6H, d, J=6.6 Hz), 1.39 (9H, s), 2.21-2.35 (1H, m), 2.43 (3H, s), 2.79 (3H, s), 2.82 (2H, d, J=7.2 Hz), 4.15 (2H, d, J=4.9 Hz), 4.38 (1H, brs), 7.10 (2H, d, J=8.1 Hz), 7.29 (2H, d, J=8.1 Hz), 9.71 (1H, s).

2) To a solution of triethyl phosphonoacetate (0.033 g, 1.5 mmol) in tetrahydrofuran (10 mL) was added sodium hydride (60% in oil, 0.060 g, 1.5 mmol) at 0° C., and the mixture was stirred for 20 min. A solution of tert-butyl{[5-formyl-2-isobutyl-6-methyl-4-(4-methylphenyl)pyridin-3-yl]methyl}carbamate (0.38 g, 0.98 mmol) in tetrahydrofuran (5 mL) was added to the reaction mixture, and the mixture was stirred at room temperature for 45 min. Ethyl acetate was added to the reaction mixture, and the mixture was washed successively with saturated brine, saturated aqueous ammonium chloride solution and saturated brine, and dried over anhydrous magnesium sulfate. The solvent was evaporated under reduced pressure and the residue was purified by silica gel column chromatography to give ethyl(2E)-3-[5-{[(tert-butoxycarbonyl)amino]methyl}-6-isobutyl-2-methyl-4-(4-methylphenyl)pyridin-3-yl]acrylate (0.44 g, yield 96%) as an oil.

¹H-NMR (CDCl₃) δ:0.98 (6H, d, J=6.6 Hz), 1.23 (3H, t, J=7.2 Hz), 1.39 (9H, s), 2.16-2.27 (1H, m), 2.40 (3H, s), 2.64 (3H, s), 2.77 (2H, d, J=7.4 Hz), 4.08-4.17 (4H, m), 4.21 (1H, brs), 5.76 (1H, d, J=16.4 Hz), 6.95 (2H, d, J=8.1 Hz), 7.23 (2H, d, J=8.1 Hz), 7.37 (1H, d, J=16.4 Hz).

3) A mixture of ethyl(2E)-3-[5-{[(tert-butoxycarbonyl)amino]methyl}-6-isobutyl-2-methyl-4-(4-methylphenyl)pyridin-3-yl]acrylate (0.12 g, 0.25 mmol) and 4N hydrogen chloride 1,4-dioxane solution (5 mL, 20 mmol) was stirred at room temperature for 10 min. The solvent was evaporated under reduced pressure, and the residue was partitioned between ethyl acetate-tetrahydrofuran and saturated aqueous sodium hydrogen carbonate. The organic layer and an extract obtained by extracting the aqueous layer with ethyl acetate-tetrahydrofuran were combined, and the mixture was dried over anhydrous magnesium sulfate. The solvent was evaporated under reduced pressure, and the residue was purified by silica gel column chromatography to give ethyl(2E)-3-[5-(aminomethyl)-6-isobutyl-2-methyl-4-(4-methylphenyl)pyridin-3-yl]acrylate (0.059 g, yield 64%).

¹H-NMR (CDCl₃) δ:0.99 (6H, d, J=6.6 Hz), 1.23 (3H, t, J=7.2 Hz), 1.30 (2H, brs), 2.18-2.33 (1H, m), 2.40 (3H, s), 2.63 (3H, s), 2.79 (2H, d, J=7.1 Hz), 3.60 (2H, s), 4.13 (2H, q, J=7.2 Hz), 5.76 (1H, d, J=16.4 Hz), 7.01 (2H, d, J=8.0 Hz), 7.24 (2H, d, J=8.0 Hz), 7.39 (1H, d, J=16.4 Hz).

### Example 9

### (2E)-3-[5-(aminomethyl)-6-isobutyl-2-methyl-4-(4-methylphenyl)pyridin-3-yl]acrylic acid dihydrochloride

1) To a solution of ethyl(2E)-3-[5-{[(tert-butoxycarbonyl)amino]methyl}-6-isobutyl-2-methyl-4-(4-methylphenyl)pyridin-3-yl]acrylate (0.32 g, 0.69 mmol) in tetrahydrofuran (10 mL) was added 1N aqueous sodium hydroxide solution (3.4 mL, 3.4 mmol), and the mixture was stirred at 60° C. for 12 hrs. The reaction mixture was acidified with 1N hydrochloric acid and extracted with ethyl acetate. The extracts were combined, and the mixture was washed with saturated brine and dried over anhydrous magnesium sulfate. The solvent was evaporated under reduced pressure and the residue was purified by silica gel column chromatography to give (2E)-3-[5-{[(tert-butoxycarbonyl)amino]methyl}-6-isobutyl-2-methyl-4-(4-methylphenyl)pyridin-3-yl]acrylic acid (0.28 g, yield 93%) as a white solid.

¹H-NMR (CDCl₃) δ:0.96 (6H, d, J=6.4 Hz), 1.39 (9H, s), 2.10-2.20 (1H, m), 2.39 (3H, s), 2.64 (3H, s), 2.79 (2H, d, J=7.2 Hz), 4.00-4.20 (2H, m), 4.34 (1H, brs), 5.76 (1H, d, J=16.4 Hz), 6.97 (2H, d, J=7.5 Hz), 7.22 (2H, d, J=7.5 Hz), 7.41 (1H, d, J=16.4 Hz).

2) (2E)-3-[5-(Aminomethyl)-6-isobutyl-2-methyl-4-(4-methylphenyl)pyridin-3-yl]acrylic acid dihydrochloride (0.077 g, yield 90%) was obtained as a white powder from (2E)-3-[5-{[(tert-butoxycarbonyl)amino]methyl}-6-isobutyl-2-methyl-4-(4-methylphenyl)pyridin-3-yl]acrylic acid (0.093 g, 0.21 mmol) according to a method similar to the method of Example 2-3).

¹H-NMR (CD₃OD) δ:1.10 (6H, d, J=6.6 Hz), 2.12-2.27 (1H, m), 2.46 (3H, brs), 2.84 (3H, s), 3.05 (2H, d, J=7.5 Hz), 4.13 (2H, s), 5.98 (1H, d, J=16.3 Hz), 7.20 (2H, d, J=8.0 Hz), 7.25 (1H, d, J=16.3 Hz), 7.46 (2H, d, J=8.0 Hz).

### Example 10

### (2E)-3-[5-(aminomethyl)-6-isobutyl-2-methyl-4-(4-methylphenyl)pyridin-3-yl]acrylamide dihydrochloride

1) tert-Butyl{[5-[(1E)-3-amino-3-oxoprop-1-en-1-yl]-2-isobutyl-6-methyl-4-(4-methylphenyl)pyridin-3-yl]methyl}carbamate (0.19 g, yield 99%) was obtained from (2E)-3-[5-{[(tert-butoxycarbonyl)amino]methyl}-6-isobutyl-2-methyl-4-(4-methylphenyl)pyridin-3-yl]acrylic acid (0.19 g, 0.43 mmol) according to a method similar to the method of Example 3-1).

¹H-NMR (CD₃OD) δ:0.97 (6H, d, J=6.6 Hz), 1.39 (9H, s), 2.09-2.20 (1H, m), 2.37 (3H, s), 2.59 (3H, s), 2.74 (2H, d, J=7.2 Hz), 3.99 (2H, s), 4.34 (1H, brs), 6.00 (1H, d, J=16.2 Hz), 7.06 (2H, d, J=8.1 Hz), 7.22-7.28 (3H, m).

2) (2E)-3-[5-(Aminomethyl)-6-isobutyl-2-methyl-4-(4-methylphenyl)pyridin-3-yl]acrylamide dihydrochloride (0.078 g, yield 99%) was obtained from tert-butyl{[5-[(1E)-3-amino-3-oxoprop-1-en-1-yl]-2-isobutyl-6-methyl-4-(4-methylphenyl)pyridin-3-yl]methyl}carbamate (0.083 g, 0.19 mmol) according to a method similar to the method of Example 2-3).

¹H-NMR (CD₃OD) δ:1.11 (6H, d, J=6.6 Hz), 2.13-2.22 (1H, m), 2.45 (3H, s), 2.87 (3H, s), 3.10 (2H, d, J=7.5 Hz), 4.15 (2H, S), 6.12 (1H, d, J=16.2 Hz), 7.11 (1H, d, J=16.2 Hz), 7.23 (2H, d, J=7.9 Hz), 7.45 (2H, d, J=7.9 Hz).

### Example 11

### methyl 5-(aminomethyl)-6-isobutyl-2-methyl-4-phenylnicotinate

1) Methyl 5-cyano-6-isobutyl-2-methyl-4-phenyl-1,4-dihydropyridine-3-carboxylate (10.7 g, yield 86%) was obtained as a white powder from 5-methyl-3-oxohexanenitrile (5.0 g, 40 mmol), benzaldehyde (4.2 g, 40 mmol) and methyl 3-aminocrotonate (4.6 g, 40 mmol) according to a method similar to the method of Example 1-2).

¹H-NMR (CDCl₃) δ:0.93 (3H, d, J=6.6 Hz), 0.99 (3H, d, J=6.6 Hz), 1.82-1.97 (1H, m), 2.18-2.34 (2H, m), 2.38 (3H, s), 3.57 (3H, s), 4.61 (1H, s), 5.69 (1H, brs), 7.18-7.32 (5H, m).

2) Methyl 5-cyano-6-isobutyl-2-methyl-4-phenylnicotinate (8.4 g, yield 80%) was obtained as a white powder from methyl 5-cyano-6-isobutyl-2-methyl-4-phenyl-1,4-dihydropyridine-3-carboxylate (10.7 g, 34 mmol) according to a method similar to the method of Example 1-3).

¹H-NMR (CDCl₃) δ:1.01 (6H, d, J=6.8 Hz), 2.21-2.35 (1H, m), 2.64 (3H, s), 2.96 (2H, d, J=7.2 Hz), 3.57 (3H, s), 7.33-7.39 (2H, m), 7.44-7.50 (3H, m).

3) Methyl 5-(aminomethyl)-6-isobutyl-2-methyl-4-phenylnicotinate (0.21 g, yield 2.5%) was obtained as a white powder from methyl 5-cyano-6-isobutyl-2-methyl-4-phenylnicotinate (8.4 g, 27 mmol) according to a method similar to the method of Example 1-4).

¹H-NMR (CDCl₃) δ:1.02 (6H, d, J=6.6 Hz), 2.17-2.33 (1H, m), 2.54 (3H, s), 2.81 (2H, d, J=7.4 Hz), 3.46 (3H, s), 3.65 (2H, s), 7.20-7.25 (2H, m), 7.38-7.46 (3H, m).

### Example 12

### methyl 5-(aminomethyl)-6-isobutyl-4-(4-methylphenyl)-2-propylnicotinate

1) A mixture of methyl 3-oxohexanoate (7.2 g, 50 mmol), ammonium acetate (19.3 g, 250 mmol), acetic acid (3.0 g, 50 mmol) and toluene (500 mL) was heated under reflux using a Dean-Stark trap for 11 hrs. The reaction mixture was concentrated under reduced pressure, and the residue was partitioned between ethyl acetate and saturated brine. The organic layer was dried over anhydrous magnesium sulfate, and the solvent was evaporated under reduced pressure to give methyl 3-aminohex-2-enoate as a colorless oil. Methyl 5-cyano-6-isobutyl-4-(4-methylphenyl)-2-propyl-1,4-dihydropyridine-3-carboxylate (11.8 g, yield 84%) was obtained as an oil from 5-methyl-3-oxohexanenitrile (5.0 g, 40 mmol), p-tolualdehyde (4.8 g, 40 mmol) and the aforementioned colorless oil of methyl 3-aminohex-2-enoate, according to a method similar to the method of Example 1-2).

¹H-NMR (CDCl₃) δ:0.93-1.05 (6H, m), 1.26 (3H, q, J=7.2 Hz), 1.59-1.69 (2H, m), 1.83-1.96 (1H, m), 2.23-2.47 (2H, m), 2.30 (3H, s), 2.69-2.74 (2H, m), 3.57 (3H, s), 4.58 (1H, s), 5.65 (1H, brs), 7.09 (2H, d, J=8.1 Hz), 7.13 (2H, d, J=8.1 Hz).

2) Methyl 5-cyano-6-isobutyl-4-(4-methylphenyl)-2-propylnicotinate (9.4 g, yield 80%) was obtained as an oil from methyl 5-cyano-6-isobutyl-4-(4-methylphenyl)-2-propyl-1,4-dihydropyridine-3-carboxylate (11.8 g, 33 mmol) according to a method similar to the method of Example 1-3).

¹H-NMR (CDCl₃) δ:0.98 (3H, t, J=7.4 Hz), 1.01 (6H, d, J=6.6 Hz), 1.73-1.85 (2H, m), 2.22-2.35 (1H, m), 2.41 (3H, s), 2.78 (2H, m), 2.96 (2H, d, J=7.4 Hz), 3.58 (3H, s), 7.23-7.32 (4H, m)

3) Methyl 5-(aminomethyl)-6-isobutyl-4-(4-methylphenyl)-2-propylnicotinate (0.78 g, yield 88%) was obtained as an oil from methyl 5-cyano-6-isobutyl-4-(4-methylphenyl)-2-propylnicotinate (0.88 g, 2.6 mmol) according to a method similar to the method of Example 1-4).

¹H-NMR (CDCl₃) δ:0.94-0.99 (9H, m), 1.70-1.83 (2H, m), 2.18-2.31 (1H, m), 2.39 (3H, s), 2.69-2.74 (2H, m), 2.81 (2H, d, J=7.2 Hz), 3.48 (3H, s), 3.65 (2H, s), 7.12 (2H, d, J=8.1 Hz), 7.21 (2H, d, J=8.1 Hz).

### Example 13

### [5-(aminomethyl)-6-isobutyl-2-methyl-4-(4-methylphenyl)pyridin-3-yl]acetic acid dihydrochloride

1) To a solution of methyl[5-{[(tert-butoxycarbonyl)amino]methyl}-6-isobutyl-2-methyl-4-(4-methylphenyl)pyridin-3-yl]acetate (0.25 g, 0.56 mmol) in tetrahydrofuran (15 mL) were added ethanol (10 mL) and 8N aqueous sodium hydroxide solution (3.0 mL, 24 mmol), and the mixture was heated under reflux for 3 hrs. The reaction mixture was acidified with 6N hydrochloric acid and extracted with ethyl acetate. The extract was washed with saturated brine and dried over anhydrous magnesium sulfate. The solvent was evaporated under reduced pressure, and the residue was purified by silica gel column chromatography to give [5-{[(tert-butoxycarbonyl)amino]methyl}-6-isobutyl-2-methyl-4-(4-methylphenyl)pyridin-3-yl]acetic acid (0.16 g, yield 65%) as a white powder.

2) [5-(Aminomethyl)-6-isobutyl-2-methyl-4-(4-methylphenyl)pyridin-3-yl]acetic acid dihydrochloride (0.15 g, yield 99%) was obtained as a white powder from [5-{[(tert-butoxycarbonyl)amino]methyl}-6-isobutyl-2-methyl-4-(4-methylphenyl)pyridin-3-yl]acetic acid (0.16 g, 0.36 mmol) according to a method similar to the method of Example 2-3).

¹H-NMR (CD₃OD) δ:1.10 (6H, d, J=6.4 Hz), 2.09-2.25 (1H, m), 2.48 (3H, s), 2.84 (3H, s), 3.10 (2H, d, J=7.4 Hz), 3.60 (2H, s), 4.09 (2H, s), 7.20 (2H, d, J=7.9 Hz), 7.49 (2H, d, J=7.9 Hz).

### Example 14

### methyl 5-(aminomethyl)-6-isobutyl-2-(2-methoxy-2-oxoethyl)-4-(4-methylphenyl)nicotinate

1) Dimethyl 3-aminopent-2-enedioate was obtained from dimethyl 1,3-acetonedicarboxylate (7.0 g, 40 mmol) according to a method similar to the method of Example 12-1).

Methyl 5-cyano-6-isobutyl-2-(2-methoxy-2-oxoethyl)-4-(4-methylphenyl)-1,4-dihydropyridine-3-carboxylate (11.5 g, yield 75%) was obtained as a yellow oil from the obtained dimethyl 3-aminopent-2-enedioate, 5-methyl-3-oxohexanenitrile (5.0 g, 40 mmol) and p-tolualdehyde (4.8 g, 40 mmol).

¹H-NMR (CDCl₃) δ:0.94 (3H, d, J=6.6 Hz), 0.98 (3H, d, J=6.6 Hz), 1.85-2.00 (1H, m), 2.20-2.40 (2H, m), 2.31 (3H, s), 3.58 (3H, s), 3.77 (3H, s), 3.85-4.10 (2H, m), 4.59 (1H, s), 7.01 (1H, brs), 7.10 (2H, d, J=8.1 Hz), 7.16 (2H, d, J=8.1 Hz).

2) Methyl 5-cyano-6-isobutyl-2-(2-methoxy-2-oxoethyl)-4-(4-methylphenyl)nicotinate (3.2 g, yield 28%) was obtained as yellow-orange oil from methyl 5-cyano-6-isobutyl-2-(2-methoxy-2-oxoethyl)-4-(4-methylphenyl)-1,4-dihydropyridine-3-carboxylate (11.5 g, 30 mmol) according to a method similar to the method of Example 1-3).

¹H-NMR (CDCl₃) δ:1.01 (6H, d, J=6.6 Hz), 2.20-2.35 (1H, m), 2.41 (3H, s), 2.97 (2H, d, J=7.2 Hz), 3.54 (3H, s), 3.71 (3H, s), 4.04 (2H, s), 7.20-7.30 (4H, m).

3) Methyl 5-(aminomethyl)-6-isobutyl-2-(2-methoxy-2-oxoethyl)-4-(4-methylphenyl)nicotinate (2.5 g, yield 77%) was obtained as a pale-yellow oil from methyl 5-cyano-6-isobutyl-2-(2-methoxy-2-oxoethyl)-4-(4-methylphenyl)nicotinate (3.2 g, 8.4 mmol) according to a method similar to the method of Example 1-4).

¹H-NMR (CDCl₃) δ:0.98 (6H, d, J=6.8 Hz), 1.39 (2H, brs), 2.15-2.35 (1H, m), 2.39 (3H, s), 2.82 (2H, d, J=7.4 Hz), 3.45 (3H, s), 3.67 (2H, s), 3.70 (3H, s), 3.94 (2H, s), 7.05-7.25 (4H, m).

### Example 15

### methyl 5-(aminomethyl)-4-(2,6-difluorophenyl)-6-isobutyl-2-methylnicotinate

1) Methyl 5-cyano-4-(2,6-difluorophenyl)-6-isobutyl-2-methyl-1,4-dihydropyridine-3-carboxylate (14.8 g, yield 36%) was obtained as yellow crystals from 5-methyl-3-oxohexanenitrile (15.0 g, 120 mmol) and 2,6-difluorobenzaldehyde (17.0 g, 120 mmol) and methyl 3-aminocrotonate (13.8 g, 120 mmol) according to a method similar to the method of Example 1-2).

¹H-NMR (CDCl₃) δ:0.95-1.05 (6H, m), 1.80-2.05 (1H, m), 2.10-2.45 (2H, m), 2.31 (3H, s), 3.56 (3H, s), 5.21 (1H, s), 5.87 (1H, brs), 6.75-6.90 (2H, m), 7.05-7.25 (1H, m).

2) Methyl 5-cyano-4-(2,6-difluorophenyl)-6-isobutyl-2-methylnicotinate (11.7 g, yield 80%) was obtained as yellow crystals from methyl 5-cyano-4-(2,6-difluorophenyl)-6-isobutyl-2-methyl-1,4-dihydropyridine-3-carboxylate (14.8 g, 43 mmol) according to a method-similar to the method of Example 1-3).

¹H-NMR (CDCl₃) δ:1.15 (6H, d, J=6.6 Hz), 2.15-2.40 (1H, m), 2.72 (3H, s), 2.97 (2H, d, J=7.0 Hz), 3.65 (3H, s), 6.95-7.10 (2H, m), 7.35-7.55 (1H, m).

3) Methyl 5-(aminomethyl)-4-(2,6-difluorophenyl)-6-isobutyl-2-methylnicotinate (9.8 g, yield 83%) was obtained as pale-yellow solid from methyl 5-cyano-4-(2,6-difluorophenyl)-6-isobutyl-2-methylnicotinate (11.7 g, 34 mmol) according to a method similar to the method of Example 1-4).

¹H-NMR (CDCl₃) δ:0.99 (6H, d, J=6.6 Hz), 1.51 (2H, brs), 2.15-2.35 (1H, m), 2.60 (3H, s), 2.83 (2H, d, J=7.5 Hz), 3.56 (3H, s), 3.62 (2H, s), 6.95-7.05 (2H, m), 7.35-7.50 (1H, m).

melting point: 48-49° C.

### Example 16

### methyl 5-(aminomethyl)-4-(4-fluorophenyl)-6-isobutyl-2-methylnicotinate

1) Methyl 5-cyano-4-(4-fluorophenyl)-6-isobutyl-2-methyl-1,4-dihydropyridine-3-carboxylate (27.4 g, yield 70%) was obtained as a yellow oil from 5-methyl-3-oxohexanenitrile (15.0 g, 120 mmol), 4-fluorobenzaldehyde (14.9 g, 120 mmol) and methyl 3-aminocrotonate (13.8 g, 120 mmol) according to a method similar to the method of Example 1-2).

2) Methyl 5-cyano-4-(4-fluorophenyl)-6-isobutyl-2-methylnicotinate (24.0 g, yield 61%) was obtained as a yellow oil from methyl 5-cyano-4-(4-fluorophenyl)-6-isobutyl-2-methyl-1,4-dihydropyridine-3-carboxylate (27 g, 82 mmol) according to a method similar to the method of Example 1-3).

1H-NMR (CDCl₃) δ:1.01 (6H, d, J=6.6 Hz), 2.15-2.40 (1H, m), 2.64 (3H, s), 2.96 (2H, d, J=7.2 Hz), 3.61 (3H, s), 7.10-7.40 (4H, m).

3) Methyl 5-(aminomethyl)-4-(4-fluorophenyl)-6-isobutyl-2-methylnicotinate (11.2 g, yield 85%) was obtained as a pale yellow solid from methyl 5-cyano-4-(4-fluorophenyl)-6-isobutyl-2-methylnicotinate (13.0 g, 40 mmol) according to a method similar to the method of Example 1-4).

¹H-NMR (CDCl₃) δ:0.98 (6H, d, J=6.6 Hz), 1.26 (2H, brs), 2.15-2.35 (1H, m), 2.54 (3H, s), 2.81 (2H, d, J=7.2 Hz), 3.51 (3H, s), 3.65 (2H, s), 7.00-7.30 (4H, m).

melting point: 55-57° C.

### Example 17

### 5-(aminomethyl)-6-isobutyl-4-(4-methylphenyl)-2-propylnicotinic acid dihydrochloride

1) Methyl 5-{[(tert-butoxycarbonyl)amino]methyl}-6-isobutyl-4-(4-methylphenyl)-2-propylnicotinate (0.71 g, yield 71%) was obtained as a white solid from methyl 5-(aminomethyl)-6-isobutyl-4-(4-methylphenyl)-2-propylnicotinate (0.78 g, 2.2 mmol) according to a method similar to the method of Example 2-1).

¹H-NMR (CDCl₃) δ:0.94-0.99 (9H, m), 1.39 (9H, s), 1.70-1.83 (2H, m), 2.16-2.27 (1H, m), 2.38 (3H, s), 2.70-2.75 (2H, m), 2.79 (2H, d, J=7.2 Hz), 3.48 (3H, s), 4.14 (2H, d, J=4.9 Hz), 4.24 (1H, brs), 7.06 (2H, d, J=7.9 Hz), 7.20 (2H, d, J=7.9 Hz).

2) 5-{[(tert-Butoxycarbonyl)amino]methyl}-6-isobutyl-4-(4-methylphenyl)-2-propylnicotinic acid (0.59 g, yield 86%) was obtained from methyl 5-{[(tert-butoxycarbonyl)amino]methyl}-6-isobutyl-4-(4-methylphenyl)-2-propylnicotinate (0.71 g, 1.6 mmol) according to a method similar to the method of Example 2-2).

¹H-NMR (CDCl₃) δ:0.94-1.05 (9H, m), 1.39 (9H, s), 1.72-1.84 (2H, m), 2.12-2.22 (1H, m), 2.38 (3H, s), 2.81-2.92 (4H, m), 4.40-4.09 (2H, m), 7.20 (2H, d, J=8.3 Hz), 7.26 (2H, d, J=8.3 Hz).

3) 5-(Aminomethyl)-6-isobutyl-4-(4-methylphenyl)-2-propylnicotinic acid dihydrochloride (0.50 g, yield 90%) was obtained as a white powder from 5-{[(tert-butoxycarbonyl)amino]methyl}-6-isobutyl-4-(4-methylphenyl)-2-propylnicotinic acid (0.59 g, 1.3 mmol) according to a method similar to the method of Example 2-3).

¹H-NMR (CD₃OD) δ:1.04-1.13 (9H, m), 1.76-1.91 (2H, m), 2.13-2.25 (1H, m), 2.44 (3H, s), 3.01-3.18 (4H, m), 4.20 (2H, brs), 7.28-7.36 (2H, m), 7.43 (2H, d, J=7.9 Hz).

### Example 18

### 5-(aminomethyl)-6-isobutyl-2-methyl-4-phenylnicotinic acid dihydrochloride

1) Methyl 5-{[(tert-butoxycarbonyl)amino]methyl}-6-isobutyl-2-methyl-4-phenylnicotinate (9.4 g, yield 83%) was obtained as a white solid from methyl 5-(aminomethyl)-6-isobutyl-2-methyl-4-phenylnicotinate (8.5 g, 27 mmol) according to a method similar to the method of Example 2-1).

¹H-NMR (CDCl₃) δ:0.98 (6H, d, J=6.6 Hz), 1.39 (9H, s), 2.15-2.20 (1H, m), 2.55 (3H, s), 2.79 (2H, d, J=7.2 Hz), 3.46 (3H, s), 4.14 (2H, d, J=4.9 Hz), 4.24 (1H, brs), 7.14-7.21 (2H, m), 7.37-7.44 (3H, m).

2) 5-{[(tert-Butoxycarbonyl)amino]methyl}-6-isobutyl-2-methyl-4-phenylnicotinic acid (0.39 g, yield 40%) was obtained as a white solid from methyl 5-{[(tert-butoxycarbonyl)amino]methyl}-6-isobutyl-2-methyl-4-phenylnicotinate (1.0 g, 2.4 mmol) according to a method similar to the method of Example 2-2).

3) 5-(Aminomethyl)-6-isobutyl-2-methyl-4-phenylnicotinic acid dihydrochloride (0.25 g, yield 86%) was obtained as a white powder from 5-{[(tert-butoxycarbonyl)amino]methyl}-6-isobutyl-2-methyl-4-phenylnicotinic acid (0.39 g, 0.98 mmol) according to a method similar to the method of Example 2-3).

¹H-NMR (CD₃OD) δ:1.04-1.15 (6H, m), 2.12-2.28 (1H, m), 2.78-2.89 (3H, m), 3.01-3.14 (2H, m), 4.13-4.20 (2H, m), 7.38-7.47 (2H, m), 7.56-7.63 (3H, m).

### Example 19

### methyl 5-((dimethylamino)methyl]-6-isobutyl-2-methyl-4-(4-methylphenyl)nicotinate

A mixture of methyl 5-(aminomethyl)-6-isobutyl-2-methyl-4-(4-methylphenyl)nicotinate (0.50 g, 1.6 mmol), formic acid (5 mL) and formalin (5 mL) was stirred at 100° C. for 12 hrs. The reaction mixture was poured into saturated aqueous sodium hydrogen carbonate, and the mixture was extracted with ethyl acetate. The extract was dried over anhydrous magnesium sulfate and the solvent was evaporated under reduced pressure. The residue was purified by silica gel column chromatography to give methyl 5-[(dimethylamino)methyl]-6-isobutyl-2-methyl-4-(4-methylphenyl)nicotinate (0.10 g, yield 19%).

¹H-NMR (CDCl₃) δ:0.98 (6H, d, J=6.8 Hz), 1.97 (6H, s), 2.14-2.28 (1H, m), 2.39 (3H, s), 2.53 (3H, s), 2.89 (2H, d, J=7.4 Hz), 3.23 (2H, s), 3.48 (3H, s), 7.04 (2H, d, J=8.0 Hz), 7.17 (2H, d, J=8.0 Hz).

### Example 20

### methyl 5-(aminomethyl)-2-methyl-6-isobutyl-[4,4′-bipyridine]-3-carboxylate

1) Methyl 5-cyano-6-isobutyl-2-methyl-1,4-dihydro-4,4′-bipyridine-3-carboxylate (26.4 g, yield 71%) was obtained as a yellow oil from 5-methyl-3-oxohexanenitrile (15.0 g, 120 mmol), isonicotinaldehyde (12.8 g, 120 mmol) and methyl 3-aminocrotonate (13.8 g, 120 mmol) according to a method similar to the method of Example 1-2).

2) To a solution of methyl 5-cyano-6-isobutyl-2-methyl-1,4-dihydro-4,4′-bipyridine-3-carboxylate (20 g, 64 mmol) in acetone (150 mL) was added diammonium cerium nitrate (45 g, 82 mmol), and the mixture was stirred at room temperature for 1 hr. The reaction mixture was cooled to 0° C. and partitioned between ethyl acetate and 2N sodium hydroxide. The organic layer and an extract obtained by extracting the aqueous layer with ethyl acetate were combined and the mixture was dried over anhydrous magnesium sulfate. The solvent was evaporated under reduced pressure, and the residue was purified by silica gel column chromatography to give methyl 5-cyano-6-isobutyl-2-methyl-4,4′-bipyridine-3-carboxylate (10.2 g, yield 51%) as a yellow oil.

3) Methyl 5-(aminomethyl)-2-methyl-6-isobutyl-[4,4′-bipyridine]-3-carboxylate (10.9 g, yield 72%) was obtained as pale-yellow solid from methyl 5-cyano-6-isobutyl-2-methyl-4,4′-bipyridine-3-carboxylate (15.0 g, 48 mmol) according to a method similar to the method of Example 1-4).

¹H-NMR (CDCl₃) δ:0.99 (6H, d, J=6.6 Hz), 1.33 (2H, brs), 2.15-2.40 (1H, m), 2.57 (3H, s), 2.82 (2H, d, J=7.2 Hz), 3.49 (3H, s), 3.61 (2H, s), 7.15-7.25 (2H, m), 8.65-8.70 (2H, m).

melting point: 63-65° C.

### Example 21

### methyl 5-(aminomethyl)-2-methyl-4-(4-methylphenyl)-6-neopentylnicotinate

1) 5,5-Dimethyl-3-oxohexanenitrile (92.0 g, yield 99%) was obtained as an oil from methyl 3,3-dimethylbutanoate (86.0 g, 0.66 mol) according to a method similar to the method of Example 1-1).

¹H-NMR (CDCl₃) δ:1.05 (9H, s), 2.49 (2H, s), 3.43 (2H, s).

2) A mixture of 5,5-dimethyl-3-oxohexanenitrile (22.0 g, 158 mmol), p-tolualdehyde (19 g, 158 mmol), piperidine (1.3 g, 15.8 mmol), acetic acid (1.9 g, 31.6 mmol) and toluene (300 mL) was heated under reflux for 12 hrs. using a Dean-Stark trap. After allowing to cool to room temperature, the reaction mixture was washed with saturated brine and dried over anhydrous magnesium sulfate. The solvent was evaporated under reduced pressure and the obtained residue was dissolved in methanol (50 mL). Methyl 3-aminocrotonate (18.2 g, 158 mmol) was added thereto and the mixture was heated under reflux for 6 hrs. The reaction mixture was concentrated under reduced pressure, and the residue was purified by silica gel column chromatography to give methyl 5-cyano-2-methyl-4-(4-methylphenyl)-6-neopentyl-1,4-dihydropyridine-3-carboxylate (23 g, yield 43%) as an oil.

¹H-NMR (CDCl₃) δ:1.01 (9H, s), 0.98 (3H, d, J=6.6 Hz), 1.80-2.00 (1H, m), 2.14-2.41 (2H, m), 2.31 (3H, s), 2.37 (3H, s), 3.58 (3H, s), 4.57 (1H, s), 5.56 (1H, brs), 7.06-7.16 (4H, m).

3) Methyl 5-cyano-2-methyl-4-(4-methylphenyl)-6-neopentylnicotinate (12 g, yield 60%) was obtained as colorless crystals from methyl 5-cyano-2-methyl-4-(4-methylphenyl)-6-neopentyl-1,4-dihydropyridine-3-carboxylate (20 g, 59.4 mmol) according to a method similar to the method of Example 1-3).

¹H-NMR (CDCl₃) δ:1.06 (9H, s), 2.41 (3H, s), 2.63 (3H, s), 3.01 (2H, s), 3.61 (3H, s), 7.26 (4H, m).

melting point: 139-140° C.

4) Methyl 5-(aminomethyl)-2-methyl-4-(4-methylphenyl)-6-neopentylnicotinate (2.3 g, yield 56%) was obtained as colorless crystals from methyl 5-cyano-2-methyl-4-(4-methylphenyl)-6-neopentylnicotinate (4 g, 11.9 mmol) according to a method similar to the method of Example 1-4).

¹H-NMR (CDCl₃) δ:1.02 (9H, s), 1.44 (2H, brs), 2.39 (3H, s), 2.53 (3H, s), 2.88 (2H, s), 3.50 (3H, s), 3.72 (2H, s), 7.12 (2H, m), 7.21 (2H, m).

melting point: 119-120° C.

### Example 22

### 5-(aminomethyl)-2-methyl-4-(4-methylphenyl)-6-neopentylnicotinic acid dihydrochloride

1) To a solution of methyl 5-(aminomethyl)-2-methyl-4-(4-methylphenyl)-6-neopentylnicotinate (1.0 g, 2.9 mmol) in tetrahydrofuran (25 mL) was added di-tert-butyl dicarbonate (0.65 g, 3.0 mmol), and the mixture was stirred at room temperature for 1 hr. 8N Aqueous sodium hydroxide solution (2 mL) and methanol (10 mL) were added to the reaction mixture, and the mixture was heated under reflux for 3 days. The reaction mixture was allowed to cool to room temperature, acidified with 1N hydrochloric acid, and extracted with ethyl acetate. The extract was washed with saturated brine and dried over anhydrous magnesium sulfate. The solvent was evaporated under reduced pressure and the residue was crystallized from diisopropyl ether to give 5-{[(tert-butoxycarbonyl)amino]methyl}-2-methyl-4-(4-methylphenyl)-6-neopentylnicotinic acid (0.5 g, yield 42%) as crystals.

¹H-NMR (CDCl₃) δ:0.88 (9H, s), 1.36 (9H, s), 2.38 (3H, s), 2.72 (3H, s), 2.88 (2H, s), 4.21 (2H, brs), 4.29 (1H, brs), 7.18 (2H, d, J=8.3 Hz), 7.23 (2H, d, J=8.3 Hz).

melting point: 216-217° C.

2) 4N Hydrogen chloride 1,4-dioxane solution (5 mL) was added to 5-{[(tert-butoxycarbonyl)amino]methyl}-2-methyl-4-(4-methylphenyl)-6-neopentylnicotinic acid (0.30 g, 0.7 mmol), and the mixture was stirred at room temperature for 17 hr. The reaction mixture was concentrated under reduced pressure and the obtained white solid was washed with diethyl ether to give 5-(aminomethyl)-2-methyl-4-(4-methylphenyl)-6-neopentylnicotinic acid dihydrochloride (0.2 g, yield 71%) as a white powder.

¹H-NMR (DMSO-d₆) δ:1.02 (9H, s), 2.37 (3H, s), 2.59 (3H, s), 3.04 (2H, s), 3.86 (2H, d, J=5.5 Hz), 7.23 (2H, d, J=8.1 Hz), 7.30 (2H, d, J=8.1 Hz), 8.24 (3H, brs).

### Example 23

### tert-butyl 5-(aminomethyl)-4-(4-chlorophenyl)-6-isobutyl-2-methylnicotinate

1) A mixture of tert-butyl acetoacetate (580 mL, 3.5 mol), 25% aqueous ammonia (1200 mL) and methanol (1000 mL) was stirred at room temperature for 14 hrs. After concentrating under reduced pressure, the reaction mixture was partitioned between ethyl acetate and water. The organic layer was dried over anhydrous magnesium sulfate, and the solvent was evaporated under reduced pressure to give tert-butyl 3-aminocrotonate (550 g, yield 99%) as a pale-yellow powder.

¹H-NMR (CDCl₃) δ:1.47 (9H, s), 1.87 (3H, s), 4.46 (1H, s).

2) tert-Butyl 4-(4-chlorophenyl)-5-cyano-6-isobutyl-2-methyl-1,4-dihydropyridine-3-carboxylate (7.6 g, yield 62%) was obtained as a white powder from 5-methyl-3-oxohexanenitrile (4.0 g, 32 mmol), 4-chlorobenzaldehyde (4.5 g, 32 mmol) and tert-butyl 3-aminocrotonate (5.0 g, 32 mmol) according to a method similar to the method of Example 1-2).

¹H-NMR (CDCl₃) δ:0.93 (3H, d, J=6.6 Hz), 0.99 (3H, d, J=6.6 Hz), 1.29 (9H, s), 1.80-1.95 (1H, m), 2.10-2.30 (2H, m), 2.34 (3H, s), 4.54 (1H, s), 5.56 (1H, brs), 7.10-7.20 (2H, m), 7.25-7.30 (2H, m).

melting point: 185-186° C.

3) To a solution of tert-butyl 4-(4-chlorophenyl)-5-cyano-6-isobutyl-2-methyl-1,4-dihydropyridine-3-carboxylate (7.6 g, 20 mmol) in acetone (200 mL) was added an aqueous solution (40 mL) of diammonium cerium nitrate (27 g, 49 mmol) at room temperature over 5 min. The reaction mixture was partitioned between ethyl acetate and water. The organic layer and an extract obtained by extracting the aqueous layer with ethyl acetate were combined, and the mixture was dried over anhydrous magnesium sulfate. The solvent was evaporated under reduced pressure and the residue was purified by silica gel column chromatography to give tert-butyl 4-(4-chlorophenyl)-5-cyano-6-isobutyl-2-methylnicotinate (7.2 g, yield 95%) as a white powder.

¹H-NMR (CDCl₃) δ:1.01 (6H, d, J=6.8 Hz), 1.27 (9H, s), 2.15-2.35 (1H, m), 2.65 (3H, s), 2.94 (2H, d, J=7.2 Hz), 7.30-7.35 (2H, m), 7.40-7.50 (2H, m).

melting point: 70-72° C.

4) A mixture of tert-butyl 4-(4-chlorophenyl)-5-cyano-6-isobutyl-2-methylnicotinate (1.0 g, 2.6 mmol), Raney-cobalt (4 mL), 25% aqueous ammonia (2 mL), tetrahydrofuran (20 mL) and methanol (40 mL) was stirred in a sealed tube under 0.5 MPa hydrogen atmosphere at room temperature for 5 hrs. The reaction mixture was filtered and the filtrate was concentrated under reduced pressure. The residue was partitioned between ethyl acetate and 10% aqueous potassium carbonate solution. The organic layer was washed with saturated brine and dried over anhydrous magnesium sulfate. The solvent was evaporated under reduced pressure and the residue was purified by silica gel column chromatography to give tert-butyl 5-(aminomethyl)-4-(4-chlorophenyl)-6-isobutyl-2-methylnicotinate (0.98 g, yield 97%) as a white powder.

¹H-NMR (CDCl₃) δ:0.98 (6H, d, J=6.8 Hz), 1.22 (9H, s), 1.42 (2H, brs), 2.15-2.30 (1H, m), 2.55 (3H, s), 2.79 (2H, d, J=7.2 Hz), 3.61 (2H, s), 7.21 (2H, d, J=8.3 Hz), 7.41 (2H, d, J=8.3 Hz).

melting point: 81-83° C.

### Example 24

### 5-(aminomethyl)-4-(4-chlorophenyl)-6-isobutyl-2-methylnicotinic acid hydrochloride

1) A mixture of tert-butyl 5-(aminomethyl)-4-(4-chlorophenyl)-6-isobutyl-2-methylnicotinate (0.60 g, 1.5 mmol) and trifluoroacetic acid (4 mL) was stirred at 50° C. for 4 hrs. The reaction mixture was concentrated under reduced pressure, and the residue was dissolved in 1,4-dioxane (4 mL). 4N Hydrogen chloride 1,4-dioxane solution (4 mL, 16 mmol) was added to the obtained solution, and the mixture was concentrated under reduced pressure. The residue was washed with diisopropyl ether to give 5-(aminomethyl)-4-(4-chlorophenyl)-6-isobutyl-2-methylnicotinic acid dihydrochloride (0.63 g, yield 99%) as a colorless oil.

2) 5-(Aminomethyl)-4-(4-chlorophenyl)-6-isobutyl-2-methylnicotinic acid dihydrochloride (0.63 g, 1.5 mmol) was dissolved in isopropanol (10 mL), and propylene oxide (0.27 g, 4.6 mmol) was added thereto. The mixture was stirred at room temperature for 3 hrs. The reaction mixture was concentrated under reduced pressure, and the obtained oil was crystallized from isopropanol-diisopropyl ether to give 5-(aminomethyl)-4-(4-chlorophenyl)-6-isobutyl-2-methylnicotinic acid hydrochloride (0.43 g, 76%) as a white powder.

¹H-NMR (DMSO-d₆) δ:0.96 (6H, d, J=6.6 Hz), 2.15-2.30 (1H, m), 2.49 (3H, s), 2.78 (2H, d, J=7.2 Hz), 3.75 (2H, s), 7.34 (2H, d, J=7.5 Hz), 7.54 (2H, d, J=7.5 Hz), 8.43 (1H, brs).

### Example 25

### tert-butyl 5-(aminomethyl)-6-isobutyl-2-isopropyl-4-(4-methylphenyl)nicotinate

1) To a solution of Meldrum's acid (14.41 g, 0.1 mol) and pyridine (16.2 mL, 0.2 mol) in dichloromethane (100 mL) was added dropwise isobutyryl chloride (13.4 mL, 0.11 mol) at 0° C. over 30 min., and the mixture was stirred at 0° C. for 2 hrs. The reaction mixture was poured into 0.5N hydrochloric acid, and the mixture was extracted with dichloromethane. The extract was washed with saturated brine and dried over anhydrous magnesium sulfate. The solvent was evaporated under reduced pressure. A mixture of the obtained residue, tert-butanol (11.2 g, 150 mmol) and toluene (100 mL) was heated under reflux for 6 hrs. After allowing to cool to room temperature, the reaction mixture was washed with saturated brine and dried over anhydrous magnesium sulfate. The solvent was evaporated under reduced pressure to give tert-butyl 4-methyl-3-oxopentanoate as a crude product (9.31 g). A mixture of the crude product (9.31 g), 25% aqueous ammonia (100 mL) and methanol (100 mL) was stirred at room temperature for 12 hrs. The reaction mixture was concentrated under reduced pressure, and partitioned between ethyl acetate and water. The organic layer was dried over anhydrous magnesium sulfate, and the solvent was evaporated under reduced pressure to give tert-butyl 3-amino-4-methylpent-2-enoate as a crude product (9.26 g).

2) tert-Butyl 5-cyano-6-isobutyl-2-isopropyl-4-(4-methylphenyl)-1,4-dihydropyridine-3-carboxylate (12.11 g, yield 76%) was obtained as colorless crystals from 5-methyl-3-oxohexanenitrile (5.0 g, 40 mmol), p-tolualdehyde (4.8 g, 40 mmol) and the crude product (9.26 g) of tert-butyl 3-amino-4-methylpent-2-enoate obtained in the aforementioned 1), according to a method similar to the method of Example 1-2).

3) tert-Butyl 5-cyano-6-isobutyl-2-isopropyl-4-(4-methylphenyl)nicotinate (2.88 g, yield 73%) was obtained as an oil from tert-butyl 5-cyano-6-isobutyl-2-isopropyl-4-(4-methylphenyl)-1,4-dihydropyridine-3-carboxylate (3.94 g, 10 mmol) according to a method similar to the method of Example 23-3).

¹H-NMR (CDCl₃) δ:1.01 (6H, d, J=6.6 Hz), 1.25 (9H, s), 1.32 (6H, d, J=6.6 Hz), 2.26-2.35 (1H, m), 2.40 (3H, s), 2.94 (2H, d, J=7.2 Hz), 3.14-3.23 (1H, m), 7.26-7.35 (4H, m).

4) tert-Butyl 5-(aminomethyl)-6-isobutyl-2-isopropyl-4-(4-methylphenyl)nicotinate (2.15 g, yield 77%) was obtained as a white powder from tert-butyl 5-cyano-6-isobutyl-2-isopropyl-4-(4-methylphenyl)nicotinate (2.74 g, 7 mmol) according to a method similar to the method of Example 1-4).

¹H-NMR (CDCl₃) δ:0.99 (6H, d, J=6.6 Hz), 1.18 (9H, s), 1.30 (6H, d, J=6.6 Hz), 1.39 (2H, brs), 2.26-2.35 (1H, m), 2.39 (3H, s), 2.78 (2H, d, J=6.9 Hz), 3.04-3.14 (1H, m), 3.60 (2H, S), 7.13 (2H, d, J=8.2 Hz), 7.20 (2H, d, J=8.2 Hz).

### Example 26

### 5-(aminomethyl)-6-isobutyl-2-isopropyl-4-(4-methylphenyl)nicotinic acid dihydrochloride

5-(Aminomethyl)-6-isobutyl-2-isopropyl-4-(4-methylphenyl)nicotinic acid dihydrochloride (0.37 g, yield 90%) was obtained as a white powder from tert-butyl 5-(aminomethyl)-6-isobutyl-2-isopropyl-4-(4-methylphenyl)nicotinate (0.40 g, 1 mmol) according to a method similar to the method of Example 24-1).

¹H-NMR (DMSO-d₆) δ:0.99 (6H, d, J=6.6 Hz), 1.03 (6H, d, J=6.6 Hz), 2.23-2.37 (4H, m), 2.85 (2H, d, J=6.9 Hz), 3.04-3.13 (1H, m), 3.77 (2H, d, J=5.4 Hz), 7.22 (2H, d, J=8.1 Hz), 7.28 (2H, d, J=8.1 Hz), 8.21 (3H, brs).

### Example 27

### tert-butyl 5-(aminomethyl)-4-(4-chlorophenyl)-2-methyl-6-neopentylnicotinate

1) tert-Butyl 4-(4-chlorophenyl)-5-cyano-2-methyl-6-neopentyl-1,4-dihydropyridine-3-carboxylate (2.5 g, yield 38%) was obtained as a white powder from 5,5-dimethyl-3-oxohexanenitrile (2.6 g, 18.0 mmol), 4-chlorobenzaldehyde (2.3 g, 16.0 mmol) and tert-butyl 3-aminocrotonate (2.5 g, 16.0 mmol) according to a method similar to the method of Example 1-2).

¹H-NMR (CDCl₃) δ:1.01 (9H, s), 1.29 (9H, s), 2.17 (1H, d, J=13.9 Hz), 2.34 (3H, s), 2.35 (1H, d, J=13.9 Hz), 4.55 (1H, s), 5.46 (1H, brs), 7.10-7.35 (4H, m).

melting point: 208-210° C.

2) tert-Butyl 4-(4-chlorophenyl)-5-cyano-2-methyl-6-neopentylnicotinate (2.1 g, yield 90%) was obtained as a pale-yellow powder from tert-butyl 4-(4-chlorophenyl)-5-cyano-2-methyl-6-neopentyl-1,4-dihydropyridine-3-carboxylate (2.4 g, 5.9 mmol) according to a method similar to the method of Example 23-3).

¹H-NMR (CDCl₃) δ:1.06 (9H, s), 1.28 (9H, s), 2.65 (3H, s), 3.00 30 (2H, s), 7.30-7.35 (2H, m), 7.45-7.50 (2H, m).

melting point: 94-95° C.

3) tert-Butyl 5-(aminomethyl)-4-(4-chlorophenyl)-2-methyl-6-neopentylnicotinate (0.93 g, yield 92%) was obtained as a white powder from tert-butyl 4-(4-chlorophenyl)-5-cyano-2-methyl-6-neopentylnicotinate (1.0 g, 2.5 mmol) according to a method similar to the method of Example 23-4).

¹H-NMR (CDCl₃) δ:1.02 (9H, s), 1.22 (9H, s), 1.43(2H, brs), 2.55 (3H, s), 2.86 (2H, s), 3.66 (2H, s), 7.15-7.25 (2H, m), 7.35-7.45 (2H, m).

melting point: 116-118° C.

### Example 28

### 5-(aminomethyl)-4-(4-chlorophenyl)-2-methyl-6-neopentylnicotinic acid dihydrochloride

5-(Aminomethyl)-4-(4-chlorophenyl)-2-methyl-6-neopentylnicotinic acid dihydrochloride (1.0 g, yield 98%) was obtained as a white powder from tert-butyl 5-(aminomethyl)-4-(4-chlorophenyl)-2-methyl-6-neopentylnicotinate (0.95 g, 2.4 mmol) according to a method similar to the method of Example 24-1).

¹H-NMR (DMSO-d₆) δ:1.02 (9H, s), 2.56 (3H, s), 2.94 (2H, s), 3.84 (2H, d, J=5.5 Hz), 7.35-7.40 (2H, m), 7.55-7.60 (2H, m), 8.20 (3H, brs).

melting point: 246-248° C.

### Example 29

### tert-butyl 5-(aminomethyl)-4-(4-chlorophenyl)-2,6-dineopentylnicotinate

1) To a solution (30 mL) of piperidine (0.94 g, 11 mmol) and acetic acid (0.66 g, 11 mmol) in isopropanol was added dropwise a solution (300 mL) of 5,5-dimethyl-3-oxohexanenitrile (17.0 g, 110 mmol) and p-chlorobenzaldehyde (15.5 g, 110 mmol) in isopropanol at room temperature over 30 min. and the mixture was stirred for 3 days. The solvent was evaporated under reduced pressure, and the residue was partitioned between ethyl acetate and saturated brine. The organic layer was dried over anhydrous magnesium sulfate and the solvent was evaporated under reduced pressure to give 3-(4-chlorophenyl)-2-(3,3-dimethylbutanoyl)acrylonitrile as a crude product (35.2 g).

2) tert-Butyl 3-amino-5,5-dimethylhex-2-enoate was obtained as a crude product (13 g) from Meldrum's acid (8.65 g, 60 mmol) and tert-butylacetyl chloride (9.2 mL, 66 mmol) according to a method similar to the method of Example 25-1).

3) tert-Butyl 4-(4-chlorophenyl)-5-cyano-2,6-dineopentyl-1,4-dihydropyridine-3-carboxylate (2.03 g, yield 15%) was obtained as a yellow oil from the crude product (11.7 g) obtained in the aforementioned 1), and the crude product (13.0 g) obtained in the aforementioned 2), according to a method similar to the method of Example 1-2). That is, the aforementioned two kinds of crude products were dissolved in methanol (40 mL) and the mixture was heated under reflux for 3.5 hrs. The reaction mixture was concentrated under reduced pressure and the residue was purified by silica gel column chromatography to give tert-butyl 4-(4-chlorophenyl)-5-cyano-2,6-dineopentyl-1,4-dihydropyridine-3-carboxylate.

¹H-NMR (CDCl₃) δ:1.00 (9H, s), 1.03 (9H, s), 1.29 (9H, s), 2.24 (4H, s), 4.58 (1H, brs), 5.37 (1H, brs), 7.20-7.32 (4H, m).

4) tert-Butyl 4-(4-chlorophenyl)-5-cyano-2,6-dineopentylnicotinate (0.75 g, yield 38%) was obtained from tert-butyl 4-(4-chlorophenyl)-5-cyano-2,6-dineopentyl-1,4-dihydropyridine-3-carboxylate (2.03 g, 4.44 mmol) according to a method similar to the method of Example 23-3).

¹H-NMR (CDCl₃) δ:1.04 (9H, s), 1.07 (9H, s), 1.24 (9H, s), 2.84 (2H, s), 3.00 (2H, s), 7.31 (2H, d, J=8.67 Hz), 7.45 (2H, d, J=8.67 Hz).

5) tert-Butyl 5-(aminomethyl)-4-(4-chlorophenyl)-2,6-dineopentylnicotinate (0.35 g, yield 46%) was obtained as a pale-yellow solid from tert-butyl 4-(4-chlorophenyl)-5-cyano-2,6-dineopentylnicotinate (0.75 g, 1.65 mmol) according to a method similar to the method of Example 23-4).

¹H-NMR (CDCl₃) δ:1.02 (9H, s), 1.04 (9H, s), 1.18 (9H, s), 2.74 (2H, s), 2.86 (2H, s), 3.64 (2H, s), 7.21 (2H, d, J=8.48 Hz), 7.40 (2H, d, J=8.48 Hz).

### Example 30

### 5-(aminomethyl)-4-(4-chlorophenyl)-2,6-dineopentylnicotinic acid dihydrochloride

5-(Aminomethyl)-4-(4-chlorophenyl)-2,6-dineopentylnicotinic acid dihydrochloride (0.21 g, yield 69%) was obtained as a white solid from tert-butyl 5-(aminomethyl)-4-(4-chlorophenyl)-2,6-dineopentylnicotinate (0.30 g, 0.653 mmol) according to a method similar to the method of Example 24-1).

¹H-NMR (CDCl₃) δ:0.99 (9H, s), 1.03 (9H, s), 2.77 (2H, s), 2.91 (2H, s), 3.83 (2H, d, J=5.65 Hz), 7.35 (2H, d, J=8.48 Hz), 7.54 (2H, d, J=8.29 Hz), 8.12 (2H, brs).

### Example 31

5-(aminomethyl)-2-methyl-4-(4-methylphenyl)-6-neopentylnicotinic acid hemifumarate (to be sometimes referred to as bis[5-(aminomethyl)-2-methyl-4-(4-methylphenyl)-6-neopentylnicotinic acid] fumarate in this specification)

1) To a mixture of 5-(aminomethyl)-2-methyl-4-(4-methylphenyl)-6-neopentylnicotinic acid dihydrochloride (5.99 g, 15.0 mmol), tetrahydrofuran (50 mL) and 1 M aqueous sodium hydroxide solution (50 mL) was added dropwise benzyl chloroformate (95%, 2.48 mL, 16.5 mmol) at room temperature. The obtained mixture was stirred for 2 hrs., and 0.1 M hydrochloric acid (100 mL) was added. The mixture was extracted with ethyl acetate-tetrahydrofuran (1:1). The organic layer was washed with water and saturated brine, dried over anhydrous magnesium sulfate and concentrated under reduced pressure. The residue was recrystallized from tetrahydrofuran to give 5-({[(benzyloxy)carbonyl]amino}methyl)-2-methyl-4-(4-methylphenyl)-6-neopentylnicotinic acid (5.57 g, 81%) as colorless powder crystals.

¹H-NMR (DMSO-d₆) δ:0.98 (9H, s), 2.33 (3H, s), 2.44 (3H, s), 2.70 (2H, s), 3.97 (2H, d, J=4.1 Hz), 4.98 (2H, s), 7.15-7.20 (4H, m), 7.27-7.42 (6H, m), 12.96 (1H, brs).

2) A mixture of 5-({[(benzyloxy)carbonyl]amino}methyl)-2-methyl-4-(4-methylphenyl)-6-neopentylnicotinic acid (5.5 g, 12 mmol), 5% palladium-carbon (11.0 g), tetrahydrofuran (100 mL) and ethanol (100 mL) was stirred overnight under a hydrogen atmosphere at room temperature. The reaction mixture was filtered, and the filtrate was concentrated under reduced pressure. The residue was recrystallized from methanol to give 5-(aminomethyl)-2-methyl-4-(4-methylphenyl)-6-neopentylnicotinic acid (2.46 g, 63%) as colorless powder crystals.

¹H-NMR (DMSO-d₆) δ:0.96 (9H, s), 2.33 (3H, s), 2.36 (3H, s), 2.76 (2H, s), 3.56 (2H, s), 7.12-7.18 (4H, m).

3) 5-(Aminomethyl)-2-methyl-4-(4-methylphenyl)-6-neopentylnicotinic acid (1.14 g, 3.50 mmol) and fumaric acid (0.203 g, 1.75 mmol) were dissolved in water (150 mL) with heating. The obtained aqueous solution was concentrated under reduced pressure. The residue was washed with ethanol and recrystallized from water to give 5-(aminomethyl)-2-methyl-4-(4-methylphenyl)-6-neopentylnicotinic acid hemifumarate (0.902 g, 67%) as colorless powder crystals.

¹H-NMR (DMSO-d₆) δ:0.97 (9H, s), 2.34 (3H, s), 2.40 (3H, s), 2.77 (2H, s), 3.65 (2H, s), 6.45 (1H, s), 7.14-7.21 (4H, m).

### Example 32

### tert-butyl 5-(aminomethyl)-6-isobutyl-2-methyl-4-(4-methylphenyl)nicotinate

1) tert-Butyl 5-cyano-6-isobutyl-2-methyl-4-(4-methylphenyl)-1,4-dihydropyridine-3-carboxylate (159 g, yield 27%) was obtained as a white solid from tert-butyl 3-aminocrotonate (253 g, 1.60 mol) according to a method similar to the method of Example 1-2). Subsequently, tert-butyl 5-cyano-6-isobutyl-2-methyl-4-(4-methylphenyl)nicotinate (40.8 g, yield 99%) was obtained as a yellow solid from tert-butyl 5-cyano-6-isobutyl-2-methyl-4-(4-methylphenyl)-1,4-dihydropyridine-3-carboxylate (41.0 g, 112 mmol) according to a method similar to the method of Example 23-3).

1H-NMR (CDCl₃) δ:1.01 (6H, d, J=6.9 Hz), 1.26 (9H, S), 2.21-2.32 (1H, m), 2.41 (3H, s), 2.64 (3H, s), 2.93 (2H, d, J=7.5 Hz), 7.18-7.32 (4H, m).

2) tert-Butyl 5-(aminomethyl)-6-isobutyl-2-methyl-4-(4-methylphenyl)nicotinate (502 g, yield 96%) was obtained as a white solid from tert-butyl 5-cyano-6-isobutyl-2-methyl-4-(4-methylphenyl)nicotinate (515 g, 1.42 mmol) according to a method similar to the method of Example 1-4).

¹H-NMR (CDCl₃) δ:0.98 (6H, d, J=6.6 Hz), 1.19 (9H, s), 2.13-2.31 (1H, m), 2.39 (3H, s), 2.56 (3H, s), 2.79 (2H. d, J=7.4 Hz), 3.64 (2H, brs), 7.13 (2H, d, J=7.9 Hz), 7.22 (2H, d, J=7.9 Hz).

### Example 33

### ({[5-(aminomethyl)-6-isobutyl-2-methyl-4-(4-methylphenyl)pyridin-3-yl]carbonyl}oxy)acetic acid dihydrochloride

1) To a solution (10 mL) of 5-{[(tert-butoxycarbonyl)amino]methyl}-6-isobutyl-2-methyl-4-(4-methylphenyl)nicotinic acid (510 mg, 1.24 mmol) in N,N-dimethylformamide were added benzyl bromoacetate (568 mg, 2.48 mmol) and potassium carbonate (343 mg, 2.48 mmol), and the mixture was stirred at room temperature for 30 min. The reaction mixture was diluted with ethyl acetate (100 mL) and washed with saturated brine. The organic layer was dried over anhydrous magnesium sulfate. The solvent was evaporated under reduced pressure and the obtained residue was purified by silica gel column chromatography to give 2-(benzyloxy)-2-oxoethyl 5-{[(tert-butoxycarbonyl)amino]methyl}-6-isobutyl-2-methyl-4-(4-methylphenyl)nicotinate (690 mg, yield 99%) as an oil.

¹H-NMR (CDCl₃) δ:0.97 (6H, d, J=6.6 Hz), 1.39 (9H, s), 2.14-2.26 (1H, m), 2.36 (3H, s), 2.59 (3H, s), 2.79 (2H, d, J=7.4 Hz), 4.11-4.17 (2H, m), 4.22 (1H, brs), 4.40 (2H, s), 5.16 (2H, s), 7.05 (2H, d, J=8.1 Hz), 7.17 (2H, d, J=7.9 Hz), 7.29-7.39 (5H, m).

2) A mixture of 2-(benzyloxy)-2-oxoethyl 5-{[(tert-butoxycarbonyl)amino]methyl}-6-isobutyl-2-methyl-4-(4-methylphenyl)nicotinate (690 mg, 1.23 mmol), palladium-carbon (10%, dry) (132 mg, 0.124 mmol) and ethanol (10 mL) was stirred under a hydrogen atmosphere at room temperature for 30 min. After filtration, the solvent was evaporated under reduced pressure to give ({[5-{[(tert-butoxycarbonyl)amino}methyl}-6-isobutyl-2-methyl-4-(4-methylphenyl)pyridin-3-yl]carbonyl}oxy)acetic acid as a crude product (580 mg).

¹H-NMR (CDCl₃) δ:0.96 (6H, d, J=6.6 Hz), 1.39 (9H, s), 2.37 (3H, s), 2.62 (3H, s), 2.81 (2H, d, J=7.0 Hz), 4.11-4.17 (2H, m), 4.30 (1H, brs), 4.36 (2H, s), 7.06 (2H, d, J=7.7 Hz), 7.19 (2H, d, J=7.7 Hz).

3) ({[5-(Aminomethyl)-6-isobutyl-2-methyl-4-(4-methylphenyl)pyridin-3-yl]carbonyl}oxy)acetic acid dihydrochloride (517 mg, yield 94%) was obtained as a white powder from the crude product (580 mg) obtained in the aforementioned 2) according to a method similar to the method of Example 2-3).

¹H-NMR (CD₃OD) δ:1.11 (6H, d, J=6.6 Hz), 2.15-2.27 (1H, m), 2.45 (3H, s), 2.94 (3H, s), 3.11 (2H, d, J=7.5 Hz), 4.20 (2H, s), 4.50 (2H, s), 7.30 (2H, d, J=8.1 Hz), 7.42 (2H, d, J=7.9 Hz).

### Example 34

### 2-amino-2-oxoethyl 5-(aminomethyl)-6-isobutyl-2-methyl-4-(4-methylphenyl)nicotinate

1) To a solution (10 mL) of 5-{[(tert-butoxycarbonyl)amino]methyl}-6-isobutyl-2-methyl-4-(4-methylphenyl)nicotinic acid (500 mg, 1.22 mmol) in N,N-dimethylformamide were added 2-iodoacetamide (673 mg, 3.64 mmol) and potassium carbonate (337 mg, 2.44 mmol) and the mixture was stirred at room temperature for 30 min. The reaction mixture was diluted with ethyl acetate (100 mL) and washed with saturated brine. The organic layer was dried over anhydrous magnesium sulfate and the solvent was evaporated under reduced pressure. The obtained residue was purified by silica gel column chromatography to give 2-amino-2-oxoethyl 5-{[(tert-butoxycarbonyl)amino]methyl}-6-isobutyl-2-methyl-4-(4-methylphenyl)nicotinate (570 mg, yield 99%) as an oil.

¹H-NMR (CDCl₃) δ:0.98 (6H, d, J=6.8 Hz), 1.39 (9H, s), 2.17-2.31 (1H, m), 2.39 (3H, s), 2.57 (3H, s), 2.80 (2H, d, J=7.2 Hz), 4.13-4.18 (2H, m), 4.23 (1H, brs), 4.40 (2H, s), 5.12 (2H, brs), 7.12 (2H, d, J=7.7 Hz), 7.25 (2H, d, J=7.9 Hz).

2) 2-Amino-2-oxoethyl 5-(aminomethyl)-6-isobutyl-2-methyl-4-(4-methylphenyl)nicotinate (370 mg, yield 82%) was obtained as an oil from 2-amino-2-oxoethyl 5-{[(tert-butoxycarbonyl)amino]methyl}-6-isobutyl-2-methyl-4-(4-methylphenyl)nicotinate (570 mg, 1.21 mmol) according to a method similar to the method of Example 8-3).

¹H-NMR (CDCl₃) δ:0.99 (6H, d, J=6.6 Hz), 2.17-2.32 (1H, m), 2.40 (3H, s), 2.57 (3H, s), 2.82 (2H, d, J=7.2 Hz), 3.70 (2H, s), 4.39 (2H, s), 5.20 (2H, brs), 7.19 (2H, d, J=8.1 Hz), 7.27 (2H, d, J=7.9 Hz).

### Example 35

### 4-ethoxy-4-oxobutyl 5-(aminomethyl)-6-isobutyl-2-methyl-4-(4-methylphenyl)nicotinate dihydrochloride

1) A mixture of 5-{[(tert-butoxycarbonyl)amino]methyl}-6-isobutyl-2-methyl-4-(4-methylphenyl)nicotinic acid (0.41 g, 1.0 mmol), ethyl 4-bromobutyrate (0.21 g, 1.1 mmol), potassium carbonate (0.15 g, 1.1 mmol) and N,N-dimethylformamide (20 mL) was stirred at room temperature for 1 hr., and the reaction mixture was partitioned between ethyl acetate and water. The organic layer was washed successively with water and saturated brine and dried over anhydrous magnesium sulfate. The solvent was evaporated under reduced pressure and the residue was purified by silica gel column chromatography to give 4-ethoxy-4-oxobutyl 5-{[(tert-butoxycarbonyl)amino]methyl}-6-isobutyl-2-methyl-4-(4-methylphenyl)nicotinate (0.45 g, yield 85%) as a white powder.

¹H-NMR (CDCl₃) δ:0.97 (6H, d, J=6.6 Hz), 1.25 (3H, t, J=7.2 Hz), 1.39 (9H, s), 1.55-1.70 (2H, m), 2.08 (2H, t, J=7.5 Hz), 2.15-2.30 (1H, m), 2.38 (3H, s), 2.54 (3H, s), 2.78 (2H, d, J=7.3 Hz), 3.95 (2H, t, J=6.2 Hz), 4.11 (2H, q, J=7.2 Hz), 4.53 (2H, d, J=5.3 Hz), 4.23 (1H, brs), 7.07 (2H, d, J=8.0 Hz), 7.21 (2H, d, J=8.0 Hz).

2) 4-Ethoxy-4-oxobutyl 5-(aminomethyl)-6-isobutyl-2-methyl-4-(4-methylphenyl)nicotinate dihydrochloride (0.12 g, yield 95%) was obtained as a white powder from 4-ethoxy-4-oxobutyl 5-{[(tert-butoxycarbonyl)amino]methyl}-6-isobutyl-2-methyl-4-(4-methylphenyl)nicotinate (0.13 g, 0.25 mmol) according to a method similar to the method of Example 2-3).

¹H-NMR (DMSO-d₆) δ:0.96 (6H, d, J=6.6 Hz), 1.17 (3H, t, J=7.2 Hz), 1.45-1.60 (2H, m), 2.05 (2H, t, J=7.4 Hz), 2.15-2.30 (1H, m), 2.36 (3H, s), 2.51 (3H, brs), 2.85 (2H, t, J=6.3 Hz), 3.82 (2H, d, J=5.7 Hz), 3.92 (2H, t, J=6.3 Hz), 4.03 (2H, q, J=7.2 Hz), 7.19 (2H, d, J=7.9 Hz), 7.28 (2H, d, J=7.9 Hz), 8.21 (3H, brs).

melting point: 193-195° C.

### Example 36

### 4-({[5-(aminomethyl)-6-isobutyl-2-methyl-4-(4-methylphenyl)pyridin-3-yl]carbonyl}oxy)butanoic acid dihydrochloride

1) 4-Ethoxy-4-oxobutyl 5-{[(tert-butoxycarbonyl)amino]methyl}-6-isobutyl-2-methyl-4-(4-methylphenyl)nicotinate (0.30 g, 0.57 mmol) was dissolved in ethanol (20 mL) and 1N aqueous sodium hydroxide solution (4.0 mL) was added. The mixture was stirred at room temperature for 1 hr. The reaction mixture was poured into 0.5N hydrochloric acid (20 mL) and the mixture was extracted with ethyl acetate. The organic layer was washed with saturated brine and dried over anhydrous magnesium sulfate. The solvent was evaporated under reduced pressure and the obtained crude crystals were recrystallized from diisopropyl ether-ethyl acetate to give 4-({[5-{[(tert-butoxycarbonyl)amino]methyl}-6-isobutyl-2-methyl-4-(4-methylphenyl)pyridin-3-yl]carbonyl}oxy)butanoic acid (0.23 g, yield 82%) as a white powder.

¹H-NMR (CDCl₃) δ:1.02 (6H, d, J=6.4 Hz), 1.39 (9H, s), 1.55-1.70 (2H, m), 2.12 (2H, t, J=7.1 Hz), 2.15-2.30 (1H, m), 2.39 (3H, s), 2.75 (3H, brs), 2.85-3.20 (2H, m), 4.00 (2H, t, J=6.2 Hz), 4.20 (2H, d, J=3.6 Hz), 4.37 (1H, brs), 7.10 (2H, d, J=7.7 Hz), 7.26 (2H, d, J=7.7 Hz).

2) 4-({[5-(Aminomethyl)-6-isobutyl-2-methyl-4-(4-methylphenyl)pyridin-3-yl]carbonyl}oxy)butanoic acid dihydrochloride (0.20 g, yield 99%) was obtained as a white powder from 4-({[5-{[(tert-butoxycarbonyl)amino]methyl}-6-isobutyl-2-methyl-4-(4-methylphenyl)pyridin-3-yl]carbonyl}oxy)butanoic acid (0.20 g, 0.40 mmol) according to a method similar to the method of Example 2-3).

¹H-NMR (DMSO-d₆) δ:0.97 (6H, d, J=6.6 Hz), 1.40-1.55 (2H, m), 2.00 (2H, t, J=7.4 Hz), 2.15-2.30 (1H, m), 2.36 (3H, s), 2.52 (3H, brs), 2.80-2.95 (2H, m), 3.83 (2H, d, J=4.3 Hz), 3.92 (2H, t, J=6.2 Hz), 7.20 (2H, d, J=7.7 Hz), 7.29 (2H, d, J=7.7 Hz), 8.29 (3H, brs).

melting point: 221-223° C.

### Example 37

### pyridin-2-ylmethyl 5-(aminomethyl)-6-isobutyl-2-methyl-4-(4-methylphenyl)nicotinate trihydrochloride

1) To a solution (15 mL) of 5-{[(tert-butoxycarbonyl)amino]methyl}-6-isobutyl-2-methyl-4-(4-methylphenyl)nicotinic acid (1.00 g, 2.42 mmol) in N,N-dimethylformamide were added 2-(bromomethyl)pyridine hydrobromide (0.92 g, 3.64 mmol) and potassium carbonate (66.9 mg, 4.84 mmol), and the mixture was stirred for 30 min. The reaction mixture was diluted with ethyl acetate (100 mL) and washed with saturated brine. The organic layer was dried over anhydrous magnesium sulfate and the solvent was evaporated under reduced pressure. The obtained residue was purified by silica gel column chromatography to give pyridin-2-ylmethyl 5-{[(tert-butoxycarbonyl)amino]methyl}-6-isobutyl-2-methyl-4-(4-methylphenyl)nicotinate (1.20 g, yield 98%) as a pale-pink solid.

¹H-NMR (CDCl₃) δ:0.97 (6H, d, J=6.6 Hz), 1.39 (9H, s), 2.14-2.25 (1H, m), 2.35 (3H, s), 2.56 (3H, s), 2.78 (2H, d, J=7.2 Hz), 4.14 (2H, brs), 4.25 (1H, brs), 5.06 (2H, s), 6.89 (1H, d, J=7.7 Hz), 7.06 (2H, d, J=7.9 Hz), 7.13 (2H, d, J=7.9 Hz), 7.17-7.22 (1H, m), 7.57 (1H, t, J=7.7 Hz), 8.52 (1H, d, J=4.7 Hz).

2) Pyridin-2-ylmethyl 5-(aminomethyl)-6-isobutyl-2-methyl-4-(4-methylphenyl)nicotinate trihydrochloride (1.22 g, yield 99%) was obtained as a pale-pink solid from pyridin-2-ylmethyl 5-{[(tert-butoxycarbonyl)amino]methyl}-6-isobutyl-2-methyl-4-(4-methylphenyl)nicotinate (1.20 g, 2.38 mmol) according to a method similar to the method of Example 2-3).

¹H-NMR (DMSO-d₆) δ:0.97 (6H, d, J=6.4 Hz), 2.17-2.28 (1H, m), 2.34 (3H, s), 2.61 (3H, s), 2.94 (2H, d, J=6.8 Hz), 3.81 (2H, d, J=4.9 Hz), 5.20 (2H, s), 7.19 (4H, s), 7.23 (1H, brs), 7.62-7.66 (1H, m), 8.06 (1H, t, J=7.9 Hz), 8.39 (3H, brs), 8.68 (1H, d, J=4.9 Hz).

### Example 38

### 2-ethoxy-1-methyl-2-oxoethyl 5-(aminomethyl)-2-methyl-4-(4-methylphenyl)-6-neopentylnicotinate dihydrochloride

1) 2-Ethoxy-1-methyl-2-oxoethyl 5-{[(tert-butoxycarbonyl)amino]methyl}-2-methyl-4-(4-methylphenyl)-6-neopentylnicotinate (0.35 g, yield 56%) was obtained as a white powder from 5-{[(tert-butoxycarbonyl)amino]methyl}-2-methyl-4-(4-methylphenyl)-6-neopentylnicotinic acid (0.5 g, 1.2 mmol) and ethyl 2-bromopropionate (0.43 g, 2.4 mmol) according to a method similar to the method of Example 33-1).

¹H-NMR (CDCl₃) δ:1.02 (9H, s), 1.11 (3H, d, J=7.0 Hz), 1.25 (3H, t, J=7.1 Hz), 1.37 (9H, s), 2.38 (3H, s), 2.62 (3H, d, J=4.9 Hz), 2.83-2.93 (2H, m), 4.17 (2H, q, J=7.0 Hz), 4.21 (3H, s), 4.82 (1H, q, J=7.1 Hz), 7.04-7.12 (2H, m), 7.19-7.21 (2H, m).

2) 2-Ethoxy-1-methyl-2-oxoethyl 5-(aminomethyl)-2-methyl-4-(4-methylphenyl)-6-neopentylnicotinate dihydrochloride (0.16 g, yield 85%) was obtained as a white powder from 2-ethoxy-1-methyl-2-oxoethyl 5-{[(tert-butoxycarbonyl)amino]methyl}-2-methyl-4-(4-methylphenyl)-6-neopentylnicotinate (0.2 g, 0.38 mmol) according to a method similar to the method of Example 22-2).

¹H-NMR (DMSO-d₆) δ:1.02 (9H, s), 1.06 (3H, d, J=7.0 Hz), 1.16 (3H, t, J=7.1 Hz), 2.37 (3H, s), 2.58 (3H, s), 2.95 (2H, s), 3.88 (2H, s), 4.11 (2H, q, J=7.0 Hz.), 4.77 (1H, q, J=7.1 Hz) 7.13-7.16 (1H, m), 7.23-7.32 (3H, m), 8.24 (3H, s).

### Example 39

### (5-methyl-2-oxo-1,3-dioxol-4-yl)methyl 5-(aminomethyl)-2-methyl-4-(4-methylphenyl)-6-neopentylnicotinate dihydrochloride

1) (5-Methyl-2-oxo-1,3-dioxol-4-yl)methyl 5-{[(tert-butoxycarbonyl)amino]methyl}-2-methyl-4-(4-methylphenyl)-6-neopentylnicotinate (0.9 g, yield 73%) was obtained as a white powder from 5-{[(tert-butoxycarbonyl)amino]methyl}-2-methyl-4-(4-methylphenyl)-6-neopentylnicotinic acid (1.0 g, 2.3 mmol) and 4-chloromethyl-5-methyl-1,3-dioxol-2-one (0.42 g, 2.8 mmol) according to a method similar to the method of Example 33-1).

¹H-NMR (CDCl₃) δ:1.01 (9H, s) 1.36 (9H, s), 1.97 (3H, s), 2.39 (3H, s), 2.53 (3H, s), 2.88 (2H, s), 4.16 (3H, s), 4.74 (2H, s), 7.02 (2H, d, J=7.8 Hz), 7.17 (2H, d, J=7.8 Hz).

2) To a solution (2 mL) of (5-methyl-2-oxo-1,3-dioxol-4-yl)methyl 5-{[(tert-butoxycarbonyl)amino]methyl}-2-methyl-4-(4-methylphenyl)-6-neopentylnicotinate (0.8 g, 1.5 mmol) in ethyl acetate was added 4N hydrogen chloride ethyl acetate solution (8 mL) and the mixture was stirred at room temperature for 4 hrs. The reaction mixture was concentrated under reduced pressure and the obtained white solid was recrystallized from methanol-ethyl acetate to give (5-methyl-2-oxo-1,3-dioxol-4-yl)methyl 5-(aminomethyl)-2-methyl-4-(4-methylphenyl)-6-neopentylnicotinate dihydrochloride (0.6 g, yield 77%) as a white powder.

¹H-NMR (DMSO-d₆) δ:1.00 (9H, s), 1.99 (3H, s), 2.34 (3H, s), 2.52 (3H, s), 2.93 (2H, s), 3.83 (2H, d, J=5.5 Hz), 4.93 (2H, s), 7.13 (2H, d, J=7.9 Hz), 7.20 (2H, d, J=7.9 Hz), 8.18 (3H, s).

### Example 40

5-(aminomethyl)-6-isobutyl-2-methyl-4-(4-methylphenyl)nicotinic acid hemifumarate (to be sometimes referred to as bis[5-(aminomethyl)-6-isobutyl-2-methyl-4-(4-methylphenyl)nicotinic acid] fumarate in this specification)

1) A mixed solution of 5-{[(tert-butoxycarbonyl)amino]methyl}-6-isobutyl-2-methyl-4-(4-methylphenyl)nicotinic acid (53.7 g, 130 mmol) and 4N hydrogen chloride 1,4-dioxane,solution (400 mL) was stirred at room temperature for 3 hrs. The precipitated solid was collected by filtration and washed with diisopropyl ether (200 mL). The obtained white solid was dissolved in isopropanol (500 mL) and the mixture was stirred at 50° C. for 30 min. The obtained mixture was allowed to cool to room temperature, and the mixture was stirred at room temperature for 1 hr. The precipitated solid was collected by filtration and washed with isopropanol (50 mL) to give 5-(aminomethyl)-6-isobutyl-2-methyl-4-(4-methylphenyl)nicotinic acid dihydrochloride propan-2-ol solvate (1:1) (46.5 g, yield 80%) as a white solid.

¹H-NMR (DMSO-d₆) δ:0.97 (6H, d, J=6.6 Hz), 1.04 (6H, d, J=6.0 Hz), 2.16-2.27 (1H, m), 2.37 (3H, s), 2.58 (3H, s), 2.90 (2H, d, J=7.0 Hz), 3.73-3.86 (3H, m), 7.23 (2H, d, J=8.1 Hz), 7.30 (2H, d, J=7.9 Hz), 8.26 (3H, brs).

2) 5-(Aminomethyl)-6-isobutyl-2-methyl-4-(4-methylphenyl)nicotinic acid dihydrochloride propan-2-ol solvate (1:1) (35.6 g, 80 mmol) was suspended in water (80 mL) and 1N aqueous sodium hydroxide solution (160 mL, 160 mmol) was added at room temperature. The mixture was stirred for 1 hr. The precipitated solid was collected by filtration and washed with ethanol (10 mL) to give 5-(aminomethyl)-6-isobutyl-2-methyl-4-(4-methylphenyl)nicotinic acid (13.3 g, yield 53%) as a white solid.

¹H-NMR (DMSO-d₆) δ:0.93 (6H, d, J=6.8 Hz), 2.14-2.25 (1H, m), 2.34 (3H, s), 2.38 (3H, s), 2.70 (2H, d, J=7.2 Hz), 3.49 (2H, s), 7.14-7.20 (4H, m).

3) 5-(Aminomethyl)-6-isobutyl-2-methyl-4-(4-methylphenyl)nicotinic acid (15.4 g, 49.3 mmol) was suspended in water (400 mL) and the mixture was heated under reflux with stirring for 30 min. Fumaric acid (3.43 g, 29.6 mmol) was added to the obtained suspension and the mixture was stirred at room temperature for 1 hr. The precipitated solid was collected by filtration and the filtrate was washed with water (50 mL) to give 5-(aminomethyl)-6-isobutyl-2-methyl-4-(4-methylphenyl)nicotinic acid hemifumarate (13.9 g, yield 76%) as white crystals.

¹H-NMR (DMSO-d₆) δ:0.93 (6H, d, J=6.6 Hz), 2.26-2.28 (1H, m), 2.35 (3H, s), 2.42 (3H, s), 2.72 (2H, d, J=7.2 Hz), 3.55 (2H, s), 6.49 (1H, s), 7.17 (2H, d, J=8.3 Hz), 7.21 (2H, d, J=8.3 Hz).

### Example 41

### 3-[5-(aminomethyl)-6-isobutyl-2-methyl-4-(4-methylphenyl)pyridin-3-yl]propionamide dihydrochloride

A mixture of tert-butyl{[5-[(1E)-3-amino-3-oxoprop-1-en-1-yl]-2-isobutyl-6-methyl-4-(4-methylphenyl)pyridin-3-yl]methyl}carbamate (97.6 mg, 0.223 mmol), 10% palladium-carbon (24 mg, 0.0223 mmol) and ethanol (5 mL) was stirred under a hydrogen atmosphere at room temperature for 16 hrs. After filtration, the solvent was evaporated under reduced pressure to give tert-butyl{[5-(3-amino-3-oxopropyl)-2-isobutyl-6-methyl-4-(4-methylphenyl)pyridin-3-yl]methyl}carbamate as a crude product. The crude product was dissolved in 4N hydrogen chloride 1,4-dioxane solution (10 mL) and the mixture was stirred at room temperature for 30 min. The solvent was evaporated under reduced pressure and the obtained white solid was washed with diisopropyl ether to give 3-[5-(aminomethyl)-6-isobutyl-2-methyl-4-(4-methylphenyl)pyridin-3-yl]propionamide dihydrochloride (72.7 mg, yield 79%) as a white powder.

¹H-NMR (CD₃OD) δ:1.09 (6H, d, J=6.2 Hz), 2.07-2.19 (1H, m), 2.24-2.29 (2H, m), 2.48 (3H, s), 2.84 (2H, t, J=7.8 Hz), 2.90 (3H, s), 3.06 (2H, d, J=7.7 Hz), 4.04 (2H, s), 7.29 (2H, d, J=7.9 Hz), 7.50 (2H, d, J=7.7 Hz).

### Example 42

### ethyl 3-[5-(aminomethyl)-6-isobutyl-2-methyl-4-(4-methylphenyl)pyridin-3-yl]propionate dihydrochloride

1) A mixture of ethyl(2E)-3-[5-{[(tert-butoxycarbonyl)amino]methyl}-6-isobutyl-2-methyl-4-(4-methylphenyl)pyridin-3-yl]acrylate (700 mg, 1.50 mmol), 10% palladium-carbon (160 mg, 0.15 mmol) and ethanol (15 mL) was stirred under a hydrogen atmosphere at room temperature for 1 hr. After filtration, the solvent was evaporated under reduced pressure and the obtained residue was purified by silica gel column chromatography to give ethyl 3-[5-{[(tert-butoxycarbonyl)amino]methyl}-6-isobutyl-2-methyl-4-(4-methylphenyl)pyridin-3-yl]propionate (480 mg, yield 68%) as a white powder.

¹H-NMR (CDCl₃) δ:0.96 (6H, d, J=6.6 Hz), 1.18 (3H, t, J=7.2 Hz), 1.38 (9H, s), 2.11-2.30 (3H, m), 2.40 (3H, s), 2.57 (3H, s), 2.62-2.68 (2H, m), 2.72 (2H, d, J=7.4 Hz), 3.96-4.07 (4H, m), 4.18 (1H, brs), 6.98 (2H, d, J=7.91), 7.24 (2H, d, J=7.9 Hz).

2) Ethyl 3-[5-(aminomethyl)-6-isobutyl-2-methyl-4-(4-methylphenyl)pyridin-3-yl]propionate dihydrochloride (58.3 mg, yield 85%) was obtained as a white powder from ethyl 3-[5-{[(tert-butoxycarbonyl)amino]methyl}-6-isobutyl-2-methyl-4-(4-methylphenyl)pyridin-3-yl]propionate (73.0 mg, 0.156 mmol) according to a method similar to the method of Example 2-3).

¹H-NMR (CD₃OD) δ:1.08 (6H, d, J=6.6 Hz), 1.17 (3H, t, J=7.2 Hz), 2.08-2.21 (1H, m), 2.34-2.39 (2H, m), 2.48 (3H, s), 2.82-2.85 (2H, m), 2.88 (3H, s), 3.05 (2H, d, J=7.5 Hz), 4.00-4.07 (4H, m), 7.27 (2H, d, J=7.9 Hz), 7.50 (2H, d, J=7.9 Hz).

### Example 43

### 3-[5-(aminomethyl)-6-isobutyl-2-methyl-4-(4-methylphenyl)pyridin-3-yl]propionic acid dihydrochloride

1) To a mixed solution (10 mL) of ethyl 3-[5-{[(tert-butoxycarbonyl)amino]methyl}-6-isobutyl-2-methyl-4-(4-methylphenyl)pyridin-3-yl]propionate (407 mg, 0.868 mmol) in tetrahydrofuran was added 1N aqueous sodium hydroxide solution (4.30 mL, 4.30 mmol) and the mixture was stirred at 50° C. for 5 hrs. The reaction mixture was neutralized with 6N hydrochloric acid (0.8 mL) and extracted with ethyl acetate. The organic layer was washed with saturated brine and dried over anhydrous magnesium sulfate. The solvent was evaporated under reduced pressure and the obtained residue was purified by silica gel column chromatography to give 3-[5-{[(tert-butoxycarbonyl)amino]methyl}-6-isobutyl-2-methyl-4-(4-methylphenyl)pyridin-3-yl]propionic acid (255 mg, yield 60%) as a yellow powder.

¹H-NMR (CD₃OD) δ:1.04 (6H, d, J=6.6 Hz), 2.05-2.17 (1H, m), 2.26-2.36 (2H, m), 2.44 (3H, s), 2.75-2.87 (5H, m), 2.97 (2H, d, J=7.5 Hz), 4.05 (2H, s), 7.17 (2H, d, J=8.1 Hz), 7.40 (2H, d, J=7.7 Hz).

2) 3-[5-(Aminomethyl)-6-isobutyl-2-methyl-4-(4-methylphenyl)pyridin-3-yl]propionic acid dihydrochloride (94.2 mg, yield 97%) was obtained as a white powder from 3-[5-{[(tert-butoxycarbonyl)amino]methyl}-6-isobutyl-2-methyl-4-(4-methylphenyl)pyridin-3-yl]propionic acid (100 mg, 0.234 mmol) according to a method similar to the method of Example 2-3).

¹H-NMR (CD₃OD) δ:1.09 (6H, d, J=6.6 Hz), 2.09-2.22 (1H, m), 2.30-2.38 (2H, m), 2.48 (3H, s), 2.80-2.88 (2H, m), 2.90 (3H, s), 3.05 (2H, d, J=7.5 Hz), 4.05 (2H, s), 7.26 (2H, d, J=7.9 Hz), 7.51 (2H, d, J=8.1 Hz).

### Example 44

### 2-[5-(aminomethyl)-6-isobutyl-4-(4-methylphenyl)-2-propylpyridin-3-yl]acetamide

1) tert-Butyl{[5-(hydroxymethyl)-2-isobutyl-4-(4-methylphenyl)-6-propylpyridin-3-yl]methyl}carbamate (1.40 g, yield 60%) was obtained as a pale-pink powder from methyl 5-{[(tert-butoxycarbonyl)amino]methyl}-6-isobutyl-4-(4-methylphenyl)-2-propylnicotinate (2.50 g, 5.50 mmol) according to a method similar to the method of Example 5-1).

¹H-NMR (CDCl₃) δ:0.96 (6H, d, J=6.6 Hz), 1.02 (3H, d, J=7.4 Hz), 1.38 (9H, s), 1.73-1.86 (2H, m), 2.14-2.28 (1H, m), 2.41 (3H, s), 2.76 (2H, d, J=7.2 Hz), 2.88-2.93 (2H, m), 4.04 (2H, d, J=5.1 Hz), 4.20 (1H, brs), 4.36 (2H, d, J=5.8 Hz), 7.06 (2H, d, J=7.9 Hz), 7.26 (2H, d, J=7.35 Hz).

2) tert-Butyl {[5-(cyanomethyl)-2-isobutyl-4-(4-methylphenyl)-6-propylpyridin-3-yl]methyl}carbamate (0.82 g, yield 67%) was obtained as an oil from tert-butyl{[5-(hydroxymethyl)-2-isobutyl-4-(4-methylphenyl)-6-propylpyridin-3-yl]methyl}carbamate (1.20 g, 2.81 mmol) according to a method similar to the method of Example 5-2).

¹H-NMR (CDCl₃) δ:0.97 (6H, d, J=6.6 Hz), 1.05 (3H, t, J=7.4 Hz), 1.38 (9H, s), 1.78-1.90 (2H, m), 2.18-2.27 (1H, m), 2.43 (3H, s), 2.77 (2H, d, J=7.4 Hz), 2.81-2.86 (2H, m), 3.33 (2H, s), 4.05-4.06 (2H, m), 4.20 (1H, brs), 7.05 (2H, d, 7.9 Hz), 7.30 (2H, d, J=7.7 Hz),

3) tert-Butyl {[5-(2-amino-2-oxoethyl)-2-isobutyl-4-(4-methylphenyl)-6-propylpyridin-3-yl]methyl}carbamate (814 mg, yield 95%) was obtained as a white powder from tert-butyl{[5-(cyanomethyl)-2-isobutyl-4-(4-methylphenyl)-6-propylpyridin-3-yl]methyl}carbamate (0.82 g, 1.88 mmol) according to a method similar to the method of Example 6-1).

¹H-NMR (CD₃OD) δ:0.98-1.05 (9H, m), 1.38 (9H, s), 1.66-1.77 (2H, m), 2.08-2.19 (1H, m), 2.39 (3H, s), 2.76-2.80 (4H, m), 3.37 (2H, s), 3.92-3.97 (2H, m), 4.59 (1H, brs), 7.70 (2H, d, J=8.1 Hz), 7.27 (2H, d, J=7.7 Hz).

4) 2-[5-(Aminomethyl)-6-isobutyl-4-(4-methylphenyl)-2-propylpyridin-3-yl]acetamide (31 mg, yield 10%) was obtained as an oil from tert-butyl{[5-(2-amino-2-oxoethyl)-2-isobutyl-4-(4-methylphenyl)-6-propylpyridin-3-yl]methyl}carbamate (300 mg, 0.84 mmol) according to a method similar to the method of Example 8-3).

¹H-NMR (CD₃OD) δ:0.99 (6H, d, J=6.6 Hz), 1.01 (3H, t, J=7.4 Hz), 1.63-1.71 (2H, m), 2.04-2.18 (1H, m), 2.40 (3H, s), 2.71-2.76 (2H, m), 2.79 (2H, d, J=7.4 Hz), 3.33 (2H, s), 3.53 (2H, S), 7.11 (2H, d, J=7.9 Hz), 7.30 (2H, d, J=7.9 Hz).

### Example 45

### tert-butyl 5-(aminomethyl)-2,6-diisobutyl-4-(4-methylphenyl)nicotinate

1) tert-Butyl 3-amino-5-methylhex-2-enoate was obtained as a crude product (10 g) from Meldrum's acid (14.41 g, 100 mmol) and isovaleryl chloride (11.5 mL, 110 mmol) according to a method similar to the method of Example 25-1).

2) tert-Butyl 5-cyano-2,6-diisobutyl-4-(4-methylphenyl)-1,4-dihydropyridine-3-carboxylate (12.11 g, yield 74%) was obtained as an oil from 5-methyl-3-oxohexanenitrile (5.0 g, 40 mmol), p-tolualdehyde (4.8 g, 40 mmol), and the crude product (9.96 g) obtained in the aforementioned 1), according to a method similar to the method of Example 1-2).

3) tert-Butyl 5-cyano-2,6-diisobutyl-4-(4-methylphenyl)nicotinate (3.39 g, yield 83%) was obtained from tert-butyl 5-cyano-2,6-diisobutyl-4-(4-methylphenyl)-1,4-dihydropyridine-3-carboxylate (4.09 g, 10 mmol) according to a method similar to the method of Example 23-3).

¹H-NMR (CDCl₃) δ:0.95 (6H, d, J=6.6 Hz), 1.00 (6H, d, J=6.6 Hz), 1.23 (9H, s), 2.19-2.33 (1H, m), 2.41 (3H, s), 2.76 (2H, d, J=7.5 Hz), 2.94 (2H, d, J=7.2 Hz), 7.20-7.35 (4H, m).

4) tert-Butyl 5-(aminomethyl)-2,6-diisobutyl-4-(4-methylphenyl)nicotinate (2.85 g, yield 86%) was obtained as an oil from tert-butyl 5-cyano-2,6-diisobutyl-4-(4-methylphenyl)nicotinate (3.25 g, 8 mmol) according to a method similar to the method of Example 1-4).

¹H-NMR (CDCl₃) δ:0.93 (6H, d, J=6.6 Hz), 0.97 (6H, d, J=6.6 Hz), 1.17 (9H, s), 1.38 (2H, brs), 2.16-2.30 (2H, m), 2.39 (3H, s), 2.67 (2H, d, J=7.5 Hz), 2.79 (2H, d, J=7.2 Hz), 3.62 (2H, s), 7.13 (2H, d, J=8.1 Hz), 7.21 (2H, d, J=8.1 Hz).

### Example 46

### 5-(aminomethyl)-2,6-diisobutyl-4-(4-methylphenyl)nicotinic acid dihydrochloride

5-(Aminomethyl)-2,6-diisobutyl-4-(4-methylphenyl)nicotinic acid dihydrochloride (0.39 g, yield 92%) was obtained as a white powder from tert-butyl 5-(aminomethyl)-2,6-diisobutyl-4-(4-methylphenyl)nicotinate (0.41 g, 1 mmol) according to a method similar to the method of Example 24-1).

¹H-NMR (DMSO-d₆) δ:0.90 (6H, d, J=6.6 Hz), 0.96 (6H, d, J=6.6 Hz), 2.16-2.29 (2H, m), 2.37 (3H, s), 2.68 (2H, d, J=7.2 Hz), 2.88 (2H, d, J=7.2 Hz), 3.79 (2H, d, J=5.1 Hz), 7.22 (2H, d, J=8.1 Hz), 7.29 (2H, d, J=8.1 Hz), 8.12 (3H, brs).

### Example 47

### ({2-isobutyl-6-methyl-4-(4-methylphenyl)-5-[(4-methylphenyl)sulfonyl]pyridin-3-yl}methyl)amine p-toluenesulfonate

1) To a suspension of sodium p-toluenesulfinate (9.0 g, 50.5 mmol) in ethanol (50 mL) was added dropwise bromoacetone (6.92 g, 50.5 mmol). The obtained mixture was heated under reflux for 30 min., allowed to cool to room temperature and partitioned between ethyl acetate and water. The organic layer was washed with saturated brine and dried over anhydrous magnesium sulfate. The solvent was evaporated under reduced pressure. The residue was purified by silica gel column chromatography to give 1-[(4-methylphenyl)sulfonyl]acetone (8.0 g, yield 75%) as a colorless oil.

¹H-NMR (CDCl₃) δ:2.41 (3H, s), 2.46 (3H, s), 4.14 (2H, s), 7.37 (2H, d, J=8.2 Hz), 7.77 (2H, d, J=8.2 Hz).

2) A mixture of 1-[(4-methylphenyl)sulfonyl]acetone (2.0 g, 9.4 mmol), p-tolualdehyde (1.14 g, 9.4 mmol), piperidine (0.093 mL, 0.94 mmol), acetic acid (0.11 mL, 1.9 mmol) and toluene (100 mL) was heated under reflux using a Dean-Stark trap for 3 hrs. The reaction mixture was allowed to cool to room temperature, washed with saturated brine and dried over anhydrous magnesium sulfate. The solvent was evaporated under reduced pressure to give 4-(4-methylphenyl)-3-[(4-methylphenyl)sulfonyl]but-3-en-2-one as a crude product (3.5 g).

3) A mixture of 5-methyl-3-oxohexanenitrile (14.3 g, 100 mmol), acetic acid (6.0 g, 10 mmol), ammonium acetate (38.5 g, 500 mmol) and toluene (200 mL) was heated under reflux using a Dean-Stark trap for 17 hrs. The reaction mixture was allowed to cool to room temperature, washed with saturated brine and dried over anhydrous magnesium sulfate. The solvent was evaporated under reduced pressure and the obtained residue was purified by silica gel column chromatography to give 3-amino-5-methylhex-2-enenitrile as a mixture (8.2 g). The mixture (0.65 g) and the crude product (1.7 g) obtained in the aforementioned 2) were dissolved in ethanol (50 mL) and the mixture was heated under reflux for 12 hrs. The reaction mixture was concentrated under reduced pressure, and the obtained residue was purified by silica gel column chromatography to give 2-isobutyl-6-methyl-4-(4-methylphenyl)-5-[(4-methylphenyl)sulfonyl]-1,4-dihydropyridine-3-carbonitrile (1.3 g, yield 64%) as a white powder.

EIMS (M+1): 421

4) 2-Isobutyl-6-methyl-4-(4-methylphenyl)-5-[(4-methylphenyl)sulfonyl]nicotinonitrile (0.77 g, yield 68%) was obtained as a white powder from 2-isobutyl-6-methyl-4-(4-methylphenyl)-5-[(4-methylphenyl)sulfonyl]-1,4-dihydropyridine-3-carbonitrile (1.13 g, 2.7 mmol) according to a method similar to the method of Example 23-3).

¹H-NMR (CDCl₃) δ:0.99 (6H, d, J=6.6 Hz), 2.20-2.35 (1H, m), 2.38 (3H, s), 2.39 (3H, s), 2.91 (2H, d, J=7.2 Hz), 3.07 (3H, s), 6.86 (2H, d, J=8.1 Hz), 7.08 (4H, d, J=8.1 Hz), 7.23 (2H, d, J=8.1 Hz).

melting point: 129-131° C.

5) ({2-Isobutyl-6-methyl-4-(4-methylphenyl)-5-[(4-methylphenyl)sulfonyl]pyridin-3-yl}methyl)amine (0.64 g, yield 93%) was obtained as a colorless oil from 2-isobutyl-6-methyl-4-(4-methylphenyl)-5-[(4-methylphenyl)sulfonyl]nicotinonitrile (0.69 g, 1.6 mmol) according to a method similar to the method of Example 1-4).

¹H-NMR (CDCl₃) δ:0.96 (6H, d, J=6.6 Hz), 1.41 (2H, brs), 2.20-2.35 (1H, m), 2.38 (6H, s), 2.79 (2H, d, J=7.2 Hz), 2.96 (3H, s), 3.40 (2H, s), 6.76 (2H, d, J=8.1 Hz), 7.03 (2H, d, J=8.3 Hz), 7.09 (2H, d, J=8.1 Hz), 7.27 (2H, d, J=8.3 Hz).

6) To a solution of ({2-isobutyl-6-methyl-4-(4-methylphenyl)-5-[(4-methylphenyl)sulfonyl]pyridin-3-yl}methyl)amine (0.64 g, 1.5 mmol) in ethanol (5 mL) was added dropwise a solution of p-toluenesulfonic acid monohydrate (0.29 g, 1.5 mmol) in ethanol (5 mL) at room temperature. The precipitated crystals were collected by filtration, washed with cold ethanol and dried to give ({2-isobutyl-6-methyl-4-(4-methylphenyl)-5-[(4-methylphenyl)sulfonyl]pyridin-3-yl}methyl)amine p-toluenesulfonate (0.57 g, yield 63%) as a white powder.

¹H-NMR (DMSO-d₆) δ:0.94 (6H, d, J=6.6 Hz), 2.15-2.30 (1H, m), 2.29 (3H, s), 2.37 (6H, s), 2.78 (2H, d, J=7.0 Hz), 2.84 (3H, s), 3.57 (2H, s), 6.87 (2H, d, J=7.9 Hz), 7.11 (4H, d, J=8.5 Hz), 7.25-7.30 (4H, m), 7.47 (2H, d, J=7.9 Hz), 7.76 (3H, brs).

melting point: 234-235° C.

### Example 48

### tert-butyl 5-(aminomethyl)-2-benzyl-6-isobutyl-4-(4-methylphenyl)nicotinate

1) tert-Butyl 3-amino-4-phenylbut-2-enoate was obtained as a crude product (16 g) from Meldrum's acid (14.41 g, 100 mmol) and phenylacetyl chloride (14.5 mL, 110 mmol) according to a method similar to the method of Example 25-1).

2) tert-Butyl 2-benzyl-5-cyano-6-isobutyl-4-(4-methylphenyl)-1,4-dihydropyridine-3-carboxylate (14.1 g, yield 79%) was obtained as an oil from 5-methyl-3-oxohexanenitrile (5.0 g, 40 mmol), p-tolualdehyde (4.8 g, 40 mmol), and the crude product (16 g) obtained in the aforementioned 1), according to a method similar to the method of Example 1-2).

3) tert-Butyl 2-benzyl-5-cyano-6-isobutyl-4-(4-methylphenyl)nicotinate (2.92 g, yield 66%) was obtained from tert-butyl 2-benzyl-5-cyano-6-isobutyl-4-(4-methylphenyl)-1,4-dihydropyridine-3-carboxylate (4.43 g, 10 mmol) according to a method similar to the method of Example 23-3).

¹H-NMR (CDCl₃) δ:0.98 (6H, d, J=6.6 Hz), 1.10 (9H, s), 2.19-2.35 (1H, m), 2.40 (3H, s), 2.94 (2H, d, J=7.2 Hz), 4.28 (2H, s), 7.16-7.32 (9H, m).

4) tert-Butyl 5-(aminomethyl)-2-benzyl-6-isobutyl-4-(4-methylphenyl)nicotinate (2.45 g, yield 55%) was obtained as an oil from tert-butyl 2-benzyl-5-cyano-6-isobutyl-4-(4-methylphenyl)nicotinate (4.40 g, 10 mmol) according to a method similar to the method of Example 1-4).

¹H-NMR (CDCl₃) δ:0.95 (6H, d, J=6.6 Hz), 1.05 (9H, s), 1.26 (2H, brs), 2.21-2.30 (1H, m), 2.38 (3H, s), 2.79 (2H, d, J=7.5 Hz), 3.62 (2H, s), 4.20 (2H, s), 7.11-7.31 (9H, m).

### Example 49

### 5-(aminomethyl)-2-benzyl-6-isobutyl-4-(4-methylphenyl)nicotinic acid dihydrochloride

5-(Aminomethyl)-2-benzyl-6-isobutyl-4-(4-methylphenyl)nicotinic acid dihydrochloride (0.38 g, yield 82%) was obtained as a white powder from tert-butyl 5-(aminomethyl)-2-benzyl-6-isobutyl-4-(4-methylphenyl)nicotinate (0.44 g, 1 mmol) according to a method similar to the method of Example 24-1).

¹H-NMR (DMSO-d₆) δ:0.93 (6H, d, J=6.3 Hz), 2.16-2.29 (1H, m), 2.37 (3H, s), 2.82 (2H, d, J=6.6 Hz), 3.77 (2H, d, J=4.8 Hz), 4.13 (2H, s), 7.15-7.31 (9H, m), 8.16 (3H, brs).

### Example 50

### 5-(aminomethyl)-6-isobutyl-4-(4-methylphenyl)-2-phenylnicotinic acid dihydrochloride

1) Ethyl 3-amino-3-phenylacrylate was obtained as a crude product (9.5 g) from ethyl 3-oxo-3-phenylpropanoate (9.61 g, 50 mmol) and ammonium acetate (19.27 g, 250 mmol) according to a method similar to the method of Example 12-1).

2) Ethyl 5-cyano-6-isobutyl-4-(4-methylphenyl)-2-phenyl-1,4-dihydropyridine-3-carboxylate (9.52 g, yield 59%) was obtained as an oil from 5-methyl-3-oxohexanenitrile (5.0 g, 40 mmol), p-tolualdehyde (4.8 g, 40 mmol) and the crude product (9.5 g) obtained in the aforementioned 1), according to a method similar to the method of Example 1-2).

3) Ethyl 5-cyano-6-isobutyl-4-(4-methylphenyl)-2-phenylnicotinate (4.11 g, yield 85%) was obtained as an oil from ethyl 5-cyano-6-isobutyl-4-(4-methylphenyl)-2-phenyl-1,4-dihydropyridine-3-carboxylate (4.81 g, 12 mmol) according to a method similar to the method of Example 23-3).

¹H-NMR (CDCl₃) δ:0.85 (3H, t, J=7.2 Hz), 1.05 (6H, d, J=6.6 Hz), 2.29-2.44 (4H, m), 3.05 (2H, d, J=7.2 Hz), 3.91 (2H, q, J=7.2 Hz), 7.26-7.33 (4H, m), 7.43-7.48 (3H, m), 7.624-7.69 (2H, m).

4) Ethyl 5-(aminomethyl)-6-isobutyl-4-(4-methylphenyl)-2-phenylnicotinate (3.63 g, yield 90%) was obtained as an oil from ethyl 5-cyano-6-isobutyl-4-(4-methylphenyl)-2-phenylnicotinate (4.40 g, 10 mmol) according to a method similar to the method of Example 1-4).

¹H-NMR (CDCl₃) δ: 0.80 (3H, t, J=7.2 Hz), 1.03 (6H, d, J=6.6 Hz), 1.36 (2H, bs), 2.29-2.42 (4H, m), 2.90 (2H, d, J=7.2 Hz), 3.70 (2H, s), 3.81 (2H, q, J=7.2 Hz), 7.17 (2H, d, J=8.1 Hz), 7.23 (2H, d, J=8.1 Hz), 7.35-7.43 (3H, m), 7.62-7.65 (2H, m).

5) A mixture of ethyl 5-(aminomethyl)-6-isobutyl-4-(4-methylphenyl)-2-phenylnicotinate (0.80 g, 2 mmol), 6N hydrochloric acid (20 mL) and acetic acid (10 mL) was heated under reflux for 3 days. The reaction mixture was concentrated under reduced pressure. Tetrahydrofuran (20 mL) and 1N aqueous sodium hydroxide solution (30 mL) were added to the residue. To the obtained mixture was added di-tert-butyl dicarbonate (0.55 mL, 2.4 mmol) and the resulting mixture was stirred at room temperature for 2 hrs. The reaction mixture was acidified with 1N hydrochloric acid and extracted with ethyl acetate. The extract was washed with saturated brine and dried over anhydrous magnesium sulfate. The solvent was evaporated under reduced pressure and the residue was purified by silicagel column chromatography to give 5-{((tert-butoxycarbonyl)amino]methyl}-6-isobutyl-4-(4-methylphenyl)-2-phenylnicotinic acid (0.38 g, 0.8 mmol) as an oil. Then, 5-(Aminomethyl)-6-isobutyl-4-(4-methylphenyl)-2-phenylnicotinic acid dihydrochloride (0.31 g, yield 88%) was obtained as a white powder from the oil according to a method similar to the method of Example 2-3).

¹H-NMR (DMSO-d₆) δ:1.01 (6H, d, J=6.6 Hz), 2.24-2.35 (1H, m), 2.38 (3H, s), 2.93 (2H, d, J=6.9 Hz), 3.82 (2H, d, J=5.1 Hz), 7.26-7.32 (4H, m), 7.44-7.52 (3H, m), 7.66-7.69 (2H, m), 8.38 (3H, brs).

### Example 51

### methyl 5-(aminomethyl)-2-ethyl-6-isobutyl-4-(4-methylphenyl)nicotinate

1) Methyl 3-aminopent-2-enoate was obtained as a crude product (6.4 g) from methyl 3-oxopentaneoate (6.50 g, 50 mmol) and ammonium acetate (19.27 g, 250 mmol) according to a method similar to the method of Example 12-1).

2) Methyl 5-cyano-2-ethyl-6-isobutyl-4-(4-methylphenyl)-1,4-dihydropyridine-3-carboxylate (4.12 g, yield 48%) was obtained as an oil from 5-methyl-3-oxohexanenitrile (5.0 g, 40 mmol), p-tolualdehyde (4.8 g, 40 mmol) and the crude product (3.2 g) obtained in the aforementioned 1), according to a method similar to the method of Example 1-2).

3) Methyl 5-cyano-2-ethyl-6-isobutyl-4-(4-methylphenyl)nicotinate (3.41 g, yield 84%) was obtained from methyl 5-cyano-2-ethyl-6-isobutyl-4-(4-methylphenyl)-1,4-dihydropyridine-3-carboxylate (4.06 g, 12 mmol) according to a method similar to the method of Example 23-3).

¹H-NMR (CDCl₃) δ:1.01 (6H, d, J=6.6 Hz), 1.32 (3H, t, J=7.5 Hz), 2.24-2.36 (1H, m), 2.41 (3H, s), 2.85 (2H, q, J=7.5 Hz), 2.96 (2H, d, J=6.9 Hz), 3.59 (3H, s), 7.24-7.30 (4H, m).

4) Methyl 5-(aminomethyl)-2-ethyl-6-isobutyl-4-(4-methylphenyl)nicotinate (2.49 g, yield 73%) was obtained as a white powder from methyl 5-cyano-2-ethyl-6-isobutyl-4-(4-methylphenyl)nicotinate (4.40 g, 10 mmol) according to a method similar to the method of Example 1-4).

¹H-NMR (CDCl₃) δ:0.98 (6H, d, J=6.6 Hz), 1.29 (3H, t, J=7.5 Hz), 2.18-2.31 (1H, m), 2.34 (3H, s), 2.77 (2H, q, J=7.5 Hz), 2.81 (2H, d, J=7.2 Hz), 3.49 (3H, s), 3.65 (2H, s), 7.11 (2H, d, J=8.0 Hz), 7.21 (2H, d, J=8.0 Hz).

### Example 52

### 5-(aminomethyl)-2-ethyl-6-isobutyl-4-(4-methylphenyl)nicotinic acid dihydrochloride

5-(Aminomethyl)-2-ethyl-6-isobutyl-4-(4-methylphenyl)nicotinic acid dihydrochloride (0.30 g, yield 82%) was obtained as a white powder from methyl 5-(aminomethyl)-2-ethyl-6-isobutyl-4-(4-methylphenyl)nicotinate (0.34 g, 1 mmol) according to a method similar to the method of Example 50-5).

¹H-NMR (DMSO-d₆) δ:0.97 (6H, d, J=6.6 Hz), 1.26 (3H, t, J=7.5 Hz), 2.17-2.26 (1H, m), 2.37 (3H, s), 2.89 (2H, q, J=7.3 Hz), 3.00 (2H, d, J=6.9 Hz), 3.81 (2H, d, J=6.0 Hz), 7.25 (2H, d, J=8.2 Hz), 7.30 (2H, d, J=8.2 Hz), 8.38 (3H, brs).

### Example 53

### 5-(aminomethyl)-2-methyl-4-(4-methylphenyl)-6-neopentylnicotinic acid maleate

To a mixed solution of 5-(aminomethyl)-2-methyl-4-(4-methylphenyl)-6-neopentylnicotinic acid (114 mg, 0.350 mmol), acetonitrile (2 mL) and water (2 mL) was added maleic acid (40.6 mg, 0.350 mmol) and the mixture was stirred at room temperature. After dissolution of maleic acid, acetonitrile (8 mL) was added, and the mixture was stirred at room temperature for 1 hr. The obtained solution was concentrated under reduced pressure, and acetonitrile (10 mL) was added to the residue. The mixture was stirred at room temperature for 1 hr. The precipitated crystals were collected by filtration to give 5-(aminomethyl)-2-methyl-4-(4-methylphenyl)-6-neopentylnicotinic acid maleate (92.6 mg, 60%) as colorless powder crystals.

¹H-NMR (DMSO-d₆) δ:1.00 (9H, s), 2.36 (3H, s), 2.49 (3H, s), 2.81 (2H, s), 3.84 (2H, s), 6.01 (2H, s), 7.17-7.21 (2H, m), 7.27-7.31 (2H, m).

### Example 54

### 5-(aminomethyl)-2-methyl-4-(4-methylphenyl)-6-neopentylnicotinic acid tartarate

To a mixed solution of 5-(aminomethyl)-2-methyl-4-(4-methylphenyl)-6-neopentylnicotinic acid (114 mg, 0.350 mmol), acetonitrile (2 mL) and water (2 mL) was added tartaric acid (40.6 mg, 0.350 mmol), and the mixture was stirred at room temperature. After dissolution of tartaric acid, acetonitrile (8 mL) was added, and the mixture was stirred at room temperature for 1 hr. The obtained solution was concentrated under reduced pressure, and acetonitrile (10 mL) was added to the residue. The mixture was stirred at room temperature for 1 hr. The precipitated crystals were collected by filtration to give 5-(aminomethyl)-2-methyl-4-(4-methylphenyl)-6-neopentylnicotinic acid tartarate (129 mg, 77%) as colorless powder crystals.

¹H-NMR (DMSO-d₆) δ:0.98 (9H, s), 2.35 (3H, s), 2.44 (3H, s), 2.79 (2H, s), 3.75 (2H, s), 3.96 (2H, s), 7.15-7.19 (2H, m), 7.21-7.25 (2H, m).

### Example 55

### tert-butyl 5-(aminomethyl)-2-isobutyl-4-(4-methylphenyl)-6-neopentylnicotinate

1) tert-Butyl 3-amino-5-methylhex-2-enoate was obtained as a crude product (10 g) from Meldrum's acid (14.41 g, 100 mmol) and isovaleryl chloride (11.5 mL, 110 mmol) according to a method similar to the method of Example 25-1).

2) tert-Butyl 5-cyano-2-isobutyl-4-(4-methylphenyl)-6-neopentyl-1,4-dihydropyridine-3-carboxylate (3.75 g, yield 22%) was obtained as an oil from 5,5-dimethyl-3-oxohexanenitrile (5.57 g, 40 mmol), p-tolualdehyde (4.81 g, 40 mmol) and the crude product (10 g) obtained in the aforementioned 1), according to a method similar to the method of Example 1-2).

3) tert-Butyl 5-cyano-2-isobutyl-4-(4-methylphenyl)-6-neopentylnicotinate (1.66 g, yield 49%) was obtained from tert-butyl 5-cyano-2-isobutyl-4-(4-methylphenyl)-6-neopentyl-1,4-dihydropyridine-3-carboxylate (3.38 g, 10 mmol) according to a method similar to the method of Example 23-3).

¹H-NMR (CDCl₃) δ:0.95 (6H, d, J=6.6 Hz), 1.06 (9H, s), 1.24 (9H, s), 2.22-2.35 (1H, m), 2.40 (3H, s), 2.76 (2H, d, J=7.2 Hz), 3.00 (2H, s), 7.19-7.35 (4H, m).

4) tert-Butyl 5-(aminomethyl)-2-isobutyl-4-(4-methylphenyl)-6-neopentylnicotinate (1.34 g, yield 89%) was obtained as white crystals from tert-butyl 5-cyano-2-isobutyl-4-(4-methylphenyl)-6-neopentylnicotinate (3.25 g, 8 mmol) according to a method similar to the method of Example 1-4).

¹H-NMR (CDCl₃) δ:0.93 (6H, d, J=6.6 Hz), 1.02 (9H, s), 1.17 (9H, s), 1.24 (2H, brs), 2.22-2.31 (1H, m), 2.39 (3H, s), 2.66 (2H, d, J=7.5 Hz), 2.87 (2H, s), 3.68 (2H, s), 7.13 (2H, d, J=8.0 Hz), 7.21 (2H, d, J=8.0 Hz).

### Example 56

### tert-butyl 5-(aminomethyl)-2-benzyl-4-(4-methylphenyl)-6-neopentylnicotinate

1) tert-Butyl 3-amino-4-phenylbut-2-enoate was obtained as a crude product (16 g) from Meldrum's acid (14.41 g, 100 mmol) and phenylacetyl chloride (14.5 mL, 110 mmol) according to a method similar to the method of Example 25-1).

2) tert-Butyl 2-benzyl-5-cyano-4-(4-methylphenyl)-6-neopentyl-1,4-dihydropyridine-3-carboxylate (12.5 g, yield 68%) was obtained as an oil from 5,5-dimethyl-3-oxohexanenitrile (5.57 g, 40 mmol), p-tolualdehyde (4.81 g, 40 mmol), and the crude product (11.6 g) obtained in the aforementioned 1), according to a method similar to the method of Example 1-2).

3) tert-Butyl 2-benzyl-5-cyano-4-(4-methylphenyl)-6-neopentylnicotinate (6.8 g, yield 100%) was obtained from tert-butyl 2-benzyl-5-cyano-4-(4-methylphenyl)-6-neopentyl-1,4-dihydropyridine-3-carboxylate (6.8 g, 10 mmol) according to a method similar to the method of Example 23-3).

4) tert-Butyl 5-(aminomethyl)-2-benzyl-4-(4-methylphenyl)-6-neopentylnicotinate (0.48 g, yield 15%) was obtained as white crystals from tert-butyl 2-benzyl-5-cyano-4-(4-methylphenyl)-6-neopentylnicotinate (3.18 g, 7 mmol) according to a method similar to the method of Example 1-4).

¹H-NMR (CDCl₃) δ:0.96 (9H, s), 1.07 (9H, s), 2.39 (3H, s), 2.85 (2H, s), 3.67 (2H, s), 4.18 (2H, s), 7.11-7.32 (9H, m).

### Example 57

### tert-butyl 5-(aminomethyl)-2-ethyl-4-(4-methylphenyl)-6-neopentylnicotinate

1) tert-Butyl 3-aminopent-2-enoate was obtained as a crude product (8.5 g) from Meldrum's acid (14.41 g, 100 mmol) and propionyl chloride (9.6 mL, 110 mmol) according to a method similar to the method of Example 25-1).

2) tert-Butyl 5-cyano-2-ethyl-4-(4-methylphenyl)-6-neopentyl-1,4-dihydropyridine-3-carboxylate (6.0 g, yield 38%) was obtained as an oil from 5,5-dimethyl-3-oxohexanenitrile (5.57 g, 40 mmol), p-tolualdehyde (4.81 g, 40 mmol) and the crude product (8.5 g) obtained in the aforementioned 1), according to a method similar to the method of Example 1-2).

3) tert-Butyl 5-cyano-2-ethyl-4-(4-methylphenyl)-6-neopentylnicotinate (2.58 g, yield 43%) was obtained as a pale-yellow solid from tert-butyl 5-cyano-2-ethyl-4-(4-methylphenyl)-6-neopentyl-1,4-dihydropyridine-3-carboxylate (5.92 g, 15 mmol) according to a method similar to the method of Example 23-3).

¹H-NMR (CDCl₃) δ:1.07 (9H, s), 1.26 (9H, s), 1.34 (3H, t, J=7.5 Hz), 2.41 (3H, s), 2.89 (2H, q, J=7.5 Hz), 3.01 (2H, s), 7.20-7.29 (4H, m).

4) tert-Butyl 5-(aminomethyl)-2-ethyl-4-(4-methylphenyl)-6-neopentylnicotinate (1.56 g, yield 65%) was obtained as an oil from tert-butyl 5-cyano-2-ethyl-4-(4-methylphenyl)-6-neopentylnicotinate (2.36 g, 6 mmol) according to a method similar to the method of Example 1-4).

¹H-NMR (CDCl₃) δ:1.03 (9H, s), 1.19 (9H, s), 1.28 (2H, brs), 1.32 (3H, t, J=7.5 Hz), 2.39 (3H, s), 2.80 (2H, q, J=7.5 Hz), 2.87 (2H, s), 3.68 (2H, s), 7.13 (2H, d, J=8.1 Hz), 7.21 (2H, d, J=8.1 Hz).

### Example 58

### 5-(aminomethyl)-2-ethyl-4-(4-methylphenyl)-6-neopentylnicotinic acid dihydrochloride

5-(Aminomethyl)-2-ethyl-4-(4-methylphenyl)-6-neopentylnicotinic acid dihydrochloride (0.37 g, yield 90%) was obtained as a white powder from tert-butyl 5-(aminomethyl)-2-ethyl-4-(4-methylphenyl)-6-neopentylnicotinate (0.39 g, 1 mmol) according to a method similar to the method of Example 24-1).

¹H-NMR (DMSO-d₆) δ:1.02 (9H, s), 1.26 (3H, t, J=7.5 Hz), 2.37 (3H, s), 2.78 (2H, q, J=7.5 Hz), 2.92 (2H, s), 3.83 (2H, d, J=5.4 Hz), 7.21 (2H, d, J=8.0 Hz), 7.29 (2H, d, J=8.0 Hz), 8.13 (3H, brs).

### Example 59

### tert-butyl 5-(aminomethyl)-4-(4-methylphenyl)-6-neopentyl-2-propylnicotinate

1) tert-Butyl 3-aminohex-2-enoate was obtained as a crude product (9.2 g) from Meldrum's acid (14.41 g, 100 mmol) and butyryl chloride (11.4 mL, 110 mmol) according to a method similar to the method of Example 25-1).

2) tert-Butyl 5-cyano-4-(4-methylphenyl)-6-neopentyl-2-propyl-1,4-dihydropyridine-3-carboxylate (10.1 g, yield 61%) was obtained as an oil from 5,5-dimethyl-3-oxohexanenitrile (5.57 g, 40 mmol), p-tolualdehyde (4.81 g, 40 mmol) and the crude product (16 g) obtained in the aforementioned 1), according to a method similar to the method of Example 1-2).

3) tert-Butyl 5-cyano-4-(4-methylphenyl)-6-neopentyl-2-propylnicotinate (5.74 g, yield 58%) was obtained as an oil from tert-butyl 5-cyano-4-(4-methylphenyl)-6-neopentyl-2-propyl-1,4-dihydropyridine-3-carboxylate (9.8 g, 24 mmol) according to a method similar to the method of Example 23-3).

¹H-NMR (CDCl₃) δ:1.00 (3H, t, J=7.5 Hz), 1.06 (9H, s), 1.26 (9H, s), 1.75-1.88 (2H, m), 2.41 (3H, s), 2.81-2.86 (2H, m), 3.00 (2H, s), 7.18-7.30 (4H, m).

4) tert-Butyl 5-(aminomethyl)-4-(4-methylphenyl)-6-neopentyl-2-propylnicotinate (3.36 g, yield 74%) was obtained as white crystals from tert-butyl 5-cyano-4-(4-methylphenyl)-6-neopentyl-2-propylnicotinate (4.47 g, 11 mmol) according to a method similar to the method of Example 1-4).

¹H-NMR (CDCl₃) δ:0.98 (3H, t, J=7.3 Hz), 1.02 (9H, s), 1.14 (2H, brs), 1.14 (9H, s), 1.73-1.86 (2H, m), 2.39 (3H, s), 2.72-2.77 (2H, m), 2.87 (2H, s), 3.68 (2H, s), 7.13 (2H, d, J=8.1 Hz), 7.21 (2H, d, J=8.1 Hz).

### Example 60

### 5-(aminomethyl)-4-(4-methylphenyl)-6-neopentyl-2-propylnicotinic acid dihydrochloride

5-(Aminomethyl)-4-(4-methylphenyl)-6-neopentyl-2-propylnicotinic acid dihydrochloride (0.38 g, yield 90%) was obtained as a white powder from tert-butyl 5-(aminomethyl)-4-(4-methylphenyl)-6-neopentyl-2-propylnicotinate (0.41 g, 1 mmol) according to a method similar to the method of Example 24-1).

¹H-NMR (DMSO-d₆) δ:0.93 (3H, t, J=7.3 Hz), 1.02 (9H, s), 1.69-1.81 (2H, m), 2.37 (3H, s), 2.74-2.79 (2H, m), 2.94 (2H, brs), 3.84 (2H, d, J=5.1 Hz), 7.22 (2H, d, J=8.0 Hz), 7.29 (2H, d, J=8.0 Hz), 8.14 (3H, brs).

### Example 61

### tert-butyl 5-(aminomethyl)-2-isopropyl-4-(4-methylphenyl)-6-neopentylnicotinate

1) tert-Butyl 3-amino-4-methylpent-2-enoate was obtained as a crude product (9.2 g) from Meldrum's acid (14.41 g, 100 mmol) and isobutyryl chloride (11.4 mL, 110 mmol) according to a method similar to the method of Example 25-1).

2) tert-Butyl 5-cyano-2-isopropyl-4-(4-methylphenyl)-6-neopentyl-1,4-dihydropyridine-3-carboxylate (4.91 g, yield 30%) was obtained as an oil from 5,5-dimethyl-3-oxohexanenitrile (5.57 g, 40 mmol), p-tolualdehyde (4.81 g, 40 mmol) and the crude product (9.2 g) obtained in the aforementioned 1), according to a method similar to the method of Example 1-2).

3) tert-Butyl 5-cyano-2-isopropyl-4-(4-methylphenyl)-6-neopentylnicotinate (2.48 g, yield 50%) was obtained from tert-butyl 5-cyano-2-isopropyl-4-(4-methylphenyl)-6-neopentyl-1,4-dihydropyridine-3-carboxylate (4.90 g, 12 mmol) according to a method similar to the method of Example 23-3).

4) tert-Butyl 5-(aminomethyl)-2-isopropyl-4-(4-methylphenyl)-6-neopentylnicotinate (1.26 g, yield 51%) was obtained as white crystals from tert-butyl 5-cyano-2-isopropyl-4-(4-methylphenyl)-6-neopentylnicotinate (3.25 g, 8 mmol) according to a method similar to the method of Example 1-4).

¹H-NMR (CDCl₃) δ:1.04 (9H, s), 1.18 (9H, s), 1.30 (6H, d, J=6.9 Hz), 1.32 (2H, brs), 2.39 (3H, s), 2.85 (2H, s), 3.04-3.13 (1H, m), 3.66 (2H, s), 7.13 (2H, d, J=8.0 Hz), 7.20 (2H, d, J=8.0 Hz).

### Example 62

### 5-(aminomethyl)-2-isopropyl-4-(4-methylphenyl)-6-neopentylnicotinic acid dihydrochloride

5-(Aminomethyl)-2-isopropyl-4-(4-methylphenyl)-6-neopentylnicotinic acid dihydrochloride (0.37 g, yield 88%) was obtained as a white powder from tert-butyl 5-(aminomethyl)-2-isopropyl-4-(4-methylphenyl)-6-neopentylnicotinate (0.42 g, 1 mmol) according to a method similar to the method of Example 24-1).

¹H-NMR (DMSO-d₆) δ:1.04 (9H, s), 1.25 (6H, d, J=6.6 Hz), 2.36 (3H, s), 2.90 (2H, s), 3.03-3.13 (1H, m), 3.81 (2H, d, J=5.4 Hz), 7.22 (2H, d, J=8.2 Hz), 7.28 (2H, d, J=8.2 Hz), 8.18 (3H, brs).

### Example 63

### 5-(aminomethyl)-2-isobutyl-4-(4-methylphenyl)-6-neopentylnicotinic acid dihydrochloride

5-(Aminomethyl)-2-isobutyl-4-(4-methylphenyl)-6-neopentylnicotinic acid dihydrochloride (0.41 g, yield 93%) was obtained as a white powder from tert-butyl 5-(aminomethyl)-2-isobutyl-4-(4-methylphenyl)-6-neopentylnicotinate (0.42 g, 1 mmol) according to a method similar to the method of Example 24-1).

¹H-NMR (DMSO-d₆) δ:0.89 (6H, d, J=6.6 Hz), 1.02 (9H, s), 2.18-2.31 (1H, m), 2.37 (3H, s), 2.66 (2H, d, J=7.2 Hz), 2.91 (2H, s), 3.84 (2H, d,J=5.1 Hz), 7.21 (2H, d, J=8.1 Hz), 7.29 (2H, d, J=8.1 Hz), 8.08 (3H, brs).

### Example 64

### 5-(aminomethyl)-2-benzyl-4-(4-methylphenyl)-6-neopentylnicotinic acid dihydrochloride

5-(Aminomethyl)-2-benzyl-4-(4-methylphenyl)-6-neopentylnicotinic acid dihydrochloride (0.43 g, yield 91%) was obtained as a white powder from tert-butyl 5-(aminomethyl)-2-benzyl-4-(4-methylphenyl)-6-neopentylnicotinate (0.45 g, 1 mmol) according to a method similar to the method of Example 24-1).

¹H-NMR (DMSO-d₆) δ:0.95 (9H, s), 2.37 (3H, s), 2.89 (2H, s), 3.82 (2H, d, J=5.4 Hz), 4.14 (2H, s), 7.18-7.31 (9H, m), 8.17 (3H, brs).

### Example 65

### methyl 5-(aminomethyl)-6-butyl-2-methyl-4-(4-methylphenyl)nicotinate dihydrochloride

1) Methyl 6-butyl-5-cyano-2-methyl-4-(4-methylphenyl)-1,4-dihydropyridine-3-carboxylate (39 g, yield 24%) was obtained as crystals from 3-oxoheptanenitrile (64 g, 500 mmol) according to a method similar to the method of Example 1-2).

¹H-NMR (CDCl₃) δ:0.92 (3H, t, J=7.3 Hz), 1.30-1.42 (2H, m), 1.49-1.60 (2H, m), 2.30 (3H, s), 2.34-2.39 (2H, m), 2.35 (3H, s), 3.58 (3H, s), 4.56 (1H, s), 5.77 (1H, s), 7.07-7.14 (4H, m)

2) Methyl 6-butyl-5-cyano-2-methyl-4-(4-methylphenyl)nicotinate (25 g, yield 65%) was obtained as crystals from methyl 6-butyl-5-cyano-2-methyl-4-(4-methylphenyl)-1,4-dihydropyridine-3-carboxylate (25 g, 77 mmol) according to a method similar to the method of Example 1-3).

¹H-NMR (CDCl₃) δ:0.97 (3H, t, J=7.3 Hz), 1.40-1.52 (2H, m), 1.74-1.84(2H, m), 2.41 (3H, s), 2.62 (3H, s), 3.04-3.09 (2H, m), 3.60 (3H, s), 7.23-7.29 (4H, m).

3) Methyl 5-(aminomethyl)-6-butyl-2-methyl-4-(4-methylphenyl)nicotinate (17 g, yield 68%) was obtained as an oil from methyl 6-butyl-5-cyano-2-methyl-4-(4-methylphenyl)nicotinate (4 g, 11.9 mmol) according to a method similar to the method of Example 1-4). The oil (3 g) was dissolved in ethyl acetate (10 mL) and 4N hydrogen chloride ethyl acetate solution (10 mL) was added. The mixture was concentrated under reduced pressure to give methyl 5-(aminomethyl)-6-butyl-2-methyl-4-(4-methylphenyl)nicotinate dihydrochloride as a powder.

¹H-NMR (DMSO-d₆) δ:0.95 (3H, t, J=7.3 Hz), 1.38-1.51 (2H, m), 1.65-1.75 (2H, m), 2.37 (3H, s), 2.53 (3H, s), 2.98-3.03 (2H, m), 3.47 (3H, s), 3.82 (2H, d, J=5.5 Hz), 7.19 (2H, d, J=8.1 Hz), 7.30 (2H, d, J=8.1 Hz), 8.38 (3H, s).

### Example 66

### 5-(aminomethyl)-6-butyl-2-methyl-4-(4-methylphenyl)nicotinic acid dihydrochloride

1) Methyl 5-{[(tert-butoxycarbonyl)amino]methyl}-6-butyl-2-methyl-4-(4-methylphenyl)nicotinate (16.3 g, yield 89%) was obtained as crystals from methyl 5-(aminomethyl)-6-butyl-2-methyl-4-(4-methylphenyl)nicotinate (14 g, 42.9 mmol) according to a method similar to the method of Example 2-1).

2) 5-{[(tert-Butoxycarbonyl)amino]methyl}-6-butyl-2-methyl-4-(4-methylphenyl)nicotinic acid (1.5 g, yield 77%) was obtained as crystals from methyl 5-{[(tert-butoxycarbonyl)amino]methyl}-6-butyl-2-methyl-4-(4-methylphenyl)nicotinate (2.0 g, 4.7 mmol) according to a method similar to the method of Example 2-2).

3) 5-(Aminomethyl)-6-butyl-2-methyl-4-(4-methylphenyl)nicotinic acid dihydrochloride (0.56 g, yield 86%) was obtained as a white powder from 5-{[(tert-butoxycarbonyl)amino]methyl}-6-butyl-2-methyl-4-(4-methylphenyl)nicotinic acid (0.7 g, 1.7 mmol) according to a method similar to the method of Example 2-3).

¹H-NMR (DMSO-d₆) δ:0.95 (3H, t, J=7.4 Hz), 1.39-1.49 (2H, m), 1.65-1.75 (2H, m), 2.37 (3H, s), 2.61 (3H, s), 3.03-3.08 (2H, m), 3.81 (2H, d, J=5.3 Hz), 7.24 (2H, d, J=8.1 Hz), 7.31 (2H, d, J=8.1 Hz), 8.40 (3H, s).

### Example 67

### methyl 5-(aminomethyl)-2-methyl-4-(4-methylphenyl)-6-propylnicotinate dihydrochloride

1) Methyl 5-cyano-2-methyl-4-(4-methylphenyl)-6-propyl-1,4-dihydropyridine-3-carboxylate (60 g, yield 39%) was obtained as an oil from 3-oxohexanenitrile (60 g, 500 mmol) according to a method similar to the method of Example 1-2).

¹H-NMR (CDCl₃) δ:0.96 (3H, t, J=7.4 Hz), 1.54-1.66 (2H, m), 2.30 (3H, s), 2.32-2.41 (2H, m), 2.35 (3H, s), 3.58 (3H, s), 4.56 (1H, s), 5.80 (1H, s), 7.09 (2H, d, J=8.1 Hz), 7.13 (2H, d, J=8.1 Hz).

2) Methyl 5-cyano-2-methyl-4-(4-methylphenyl)-6-propylnicotinate (34.8 g, yield 58%) was obtained as crystals from methyl 5-cyano-2-methyl-4-(4-methylphenyl)-6-propyl-1,4-dihydropyridine-3-carboxylate (60 g, 193 mmol) according to a method similar to the method of Example 1-3).

¹H-NMR (CDCl₃) δ:1.05 (3H, t, J=7.4 Hz), 1.79-1.91 (2H, m), 2.41 (3H, s), 2.62 (3H, s), 3.02-3.07 (2H, m), 3.60 (3H, s), 7.23-7.29 (4H, m).

3) Methyl 5-(aminomethyl)-2-methyl-4-(4-methylphenyl)-6-propylnicotinate (15 g, yield 67%) was obtained as an oil from methyl 5-cyano-2-methyl-4-(4-methylphenyl)-6-propylnicotinate (22 g, 71.3 mmol) according to a method similar to the method of Example 1-4). The oil (2 g) was dissolved in ethyl acetate (10 mL) and 4N hydrogen chloride ethyl acetate solution (10 mL) was added. The mixture was concentrated under reduced pressure to give methyl 5-(aminomethyl)-2-methyl-4-(4-methylphenyl)-6-propylnicotinate dihydrochloride as a powder.

¹H-NMR (DMSO-d₆) δ:1.02 (3H, t, J=7.4 Hz), 1.69-1.82 (2H, m), 2.37 (3H, s), 2.53 (3H, s), 2.96-3.02 (2H, m), 3.47 (3H, s), 3.82 (2H, d, J=5.5 Hz), 7.19 (2H, d, J=8.1 Hz), 7.31 (2H, d, J=8.1 Hz), 8.38 (3H, s).

### Example 68

### 5-(aminomethyl)-2-methyl-4-(4-methylphenyl)-6-propylnicotinic acid dihydrochloride

1) Methyl 5-{[(tert-butoxycarbonyl)amino]methyl}-2-methyl-4-(4-methylphenyl)-6-propylnicotinate (12 g, yield 70%) was obtained as crystals from methyl 5-(aminomethyl)-2-methyl-4-(4-methylphenyl)-6-propylnicotinate (13 g, 41.6 mmol) according to a method similar to the method of Example 2-1).

¹H-NMR (CDCl₃) δ:1.03 (3H, t, J=7.4 Hz), 1.39 (9H, s), 1.72-1.79 (2H, m), 2.38 (3H, s), 2.53 (3H, s), 2.84-2.90 (2H, m), 3.49 (3H, s), 4.15 (2H, d, J=5.1 Hz), 4.25 (1H, s), 7.05 (2H, d, J=8.1 Hz), 7.20 (2H, d, J=8.1 Hz).

2) 5-{[(tert-Butoxycarbonyl)amino]methyl}-2-methyl-4-(4-methylphenyl)-6-propylnicotinic acid (1.6 g, yield 83%) was obtained as crystals from methyl 5-{[(tert-butoxycarbonyl)amino]methyl}-2-methyl-4-(4-methylphenyl)-6-propylnicotinate (2 g, 4.8 mmol) according to a method similar to the method of Example 2-2).

¹H-NMR (DMSO-d₆) δ:0.96 (3H, t, J=7.4 Hz), 1.35 (9H, s), 1.64-1.76 (2H, m), 2.33 (3H, s), 2.44 (3H, s) 2.67-2.72 (2H, m), 3.87 (2H, d, J=4.5 Hz), 6.99 (1H, s), 7.16-7.22 (4H, m), 12.92 (1H, s).

3) 5-(Aminomethyl)-2-methyl-4-(4-methylphenyl)-6-propylnicotinic acid dihydrochloride (0.75 g, yield 96%) was obtained as a white powder from 5-{[(tert-butoxycarbonyl)amino]methyl}-2-methyl-4-(4-methylphenyl)-6-propylnicotinic acid (0.7 g, 2.1 mmol) according to a method similar to the method of Example 2-3).

¹H-NMR (DMSO-d₆) δ:1.02 (3H, t, J=7.4 Hz), 1.69-1.82 (2H, m), 2.37 (3H, s), 2.62 (3H, s), 3.01-3.07 (2H, m), 3.82 (2H, d, J=5.3 Hz), 7.24 (2H, d, J=8.1 Hz), 7.31 (2H, d, J=8.1 Hz), 8.41 (3H, s).

### Example 69

### 5-(aminomethyl)-4-(4-fluorophenyl)-6-isobutyl-2-methylnicotinic acid dihydrochloride

1) Methyl 5-{[(tert-butoxycarbonyl)amino]methyl}-4-(4-fluorophenyl)-6-isobutyl-2-methylnicotinate (2.60 g, yield 99%) was obtained as a white solid from methyl 5-(aminomethyl)-4-(4-fluorophenyl)-6-isobutyl-2-methylnicotinate (2.00 g, 6.05 mmol) according to a method similar to the method of Example 2-1).

¹H-NMR (CDCl₃) δ:0.97 (6H, d, J=6.8 Hz), 1.39 (9H, s), 2.16-2.26 (1H, m), 2.54 (3H, s), 2.78 (2H, d, J=7.2 Hz), 3.51 (3H, s), 4.08-4.17 (2H, m), 4.22 (1H, brs), 7.07-7.20 (4H, m).

2) 5-{[(tert-Butoxycarbonyl)amino]methyl}-4-(4-fluorophenyl)-6-isobutyl-2-methylnicotinic acid (2.01 g, yield 79%) was obtained as a yellow solid from methyl 5-{[(tert-butoxycarbonyl)amino]methyl}-4-(4-fluorophenyl)-6-isobutyl-2-methylnicotinate (2.60 g, 6.24 mmol) according to a method similar to the method of Example 2-2).

¹H-NMR (CD₃OD) δ:1.04 (6H, d, J=6.6 Hz), 1.38 (9H, s), 2.12-2.22 (1H, m), 2.71 (3H, s), 2.94 (2H, d, J=7.4 Hz), 4.13 (2H, s), 7.17-7.25 (2H, m), 7.32-7.39 (2H, m).

3) 5-(Aminomethyl)-4-(4-fluorophenyl)-6-isobutyl-2-methylnicotinic acid dihydrochloride (0.20 g, yield 76%) was obtained as a white solid from 5-{[(tert-butoxycarbonyl)amino]methyl}-4-(4-fluorophenyl)-6-isobutyl-2-methylnicotinic acid (0.28 g, 0.673 mmol) according to a method similar to the method of Example 2-3).

¹H-NMR (CD₃OD) δ:1.04-1.13 (6H, m), 2.13-2.28 (1H, m), 2.78-2.86 (3H, m), 3.02-3.11 (2H, m), 4.13-4.20 (2H, m), 7.30-7.38 (2H, m), 7.42-7.51 (2H, m).

### Example 70

### 5-(aminomethyl)-4-(2,6-difluorophenyl)-6-isobutyl-2-methylnicotinic acid dihydrochloride

1) Methyl 5-{[(tert-butoxycarbonyl)amino]methyl}-4-(2,6-difluorophenyl)-6-isobutyl-2-methylnicotinate (2.49 g, yield 87%) was obtained as a white solid from methyl 5-(aminomethyl)-4-(2,6-difluorophenyl)-6-isobutyl-2-methylnicotinate (2.00 g, 6.38 mmol) according to a method similar to the method of Example 2-1).

¹H-NMR (CDCl₃) δ:0.97 (6H, d, J=6.8 Hz), 1.39 (9H, s), 2.16-2.27 (1H, m), 2.61 (3H, s), 2.79 (2H, d, J=7.4 Hz), 3.57 (3H, s), 4.13 (2H, d, J=5.3 Hz), 4.36 (1H, brs), 6.97-7.02 (2H, m), 7.34-7.44 (1H, m).

2) 5-([(tert-Butoxycarbonyl)amino]methyl}-4-(2,6-difluorophenyl)-6-isobutyl-2-methylnicotinic acid (2.22 g, yield 92%) was obtained as a yellow solid from methyl 5-{[(tert-butoxycarbonyl)amino]methyl}-4-(2,6-difluorophenyl)-6-isobutyl-2-methylnicotinate (2.49 g, 5.55 mmol) according to a method similar to the method of Example 2-2).

¹H-NMR (CDCl₃) δ:0.96 (6H, d, J=6.8 Hz), 1.39 (9H, s), 2.11-2.26 (1H, m), 2.64 (3H, s), 2.81 (2H, d, J=7.2 Hz), 4.11-4.16 (2H, m), 4.37 (1H, brs), 6.96-7.01 (2H, m), 7.34-7.43 (1H, m).

3) 5-(Aminomethyl)-4-(2,6-difluorophenyl)-6-isobutyl-2-methylnicotinic acid dihydrochloride (185 mg, yield 70%) was obtained as a white solid from 5-{[(tert-butoxycarbonyl)amino]methyl}-4-(2,6-difluorophenyl)-6-isobutyl-2-methylnicotinic acid (0.28 g, 0.635 mmol) according to a method similar to the method of Example 2-3).

¹H-NMR (CD₃OD) δ:1.08 (6H, d, J=6.8 Hz), 2.19-2.29 (1H, m), 2.81-2.88 (3H, m), 2.98-3.08 (2H, m), 4.09-4.16 (2H, m), 7.20-7.27 (2H, m), 7.64-7.72 (1H, m).

### Example 71

### tert-butyl 5-(aminomethyl)-6-isobutyl-2-methyl-4-[4-(trifluoromethyl)phenyl]nicotinate

1) 2-(3-Methylbutanoyl)-3-[4-(trifluoromethyl)phenyl]acrylonitrile was obtained as a crude product (9.8 g) from 5-methyl-3-oxohexanenitrile (4.0 g, 32 mmol) and 4-(trifluoromethyl)benzaldehyde (5.6 g, 32 mmol) according to a method similar to the method of Example 29-1).

2) tert-Butyl 5-cyano-6-isobutyl-2-methyl-4-[4-(trifluoromethyl)phenyl]-1,4-dihydropyridine-3-carboxylate (4.8 g, yield 36%) was obtained as a white powder from the crude product (9.8 g) obtained in the aforementioned 1) and tert-butyl 3-aminocrotonate (5.47 g, 35 mmol) according to a method similar to the method of Example 1-2). That is, the aforementioned crude product and tert-butyl 3-aminocrotonate were dissolved in methanol (200 mL) and the mixture was heated under reflux for 1 hr. The reaction mixture was concentrated under reduced pressure and the residue was purified by silica gel column chromatography to give tert-butyl 5-cyano-6-isobutyl-2-methyl-4-[4-(trifluoromethyl)phenyl]-1,4-dihydropyridine-3-carboxylate.

¹H-NMR (CDCl₃) δ:0.93 (3H, d, J=6.6 Hz), 0.99 (3H, d, J=6.5 Hz), 1.28 (9H, s), 1.75-2.00 (1H, m), 2.10-2.35 (2H, m), 2.36 (3H, s), 4.64 (1H, s), 5.60 (1H, brs), 7.36 (2H, d, J=8.1 Hz), 7.56 (2H, d, J=8.1 Hz).

melting point: 199-201° C.

3) tert-Butyl 5-cyano-6-isobutyl-2-methyl-4-[4-(trifluoromethyl)phenyl]nicotinate (3.5 g, yield 76%) was obtained as a white powder from tert-butyl 5-cyano-6-isobutyl-2-methyl-4-[4-(trifluoromethyl)phenyl]-1,4-dihydropyridine-3-carboxylate (4.7 g, 11 mmol) according to a method similar to the method of Example 23-3).

¹H-NMR (CDCl₃) δ:1.02 (6H, d, J=6.6 Hz), 1.23 (9H, s), 2.20-2.40 (1H, m), 2.67 (3H, s), 2.95 (2H, d, J=7.4 Hz), 7.51 (2H, d, J=8.2 Hz), 7.76 (2H, d, J=8.2 Hz).

melting point: 108-110° C.

4) tert-Butyl 5-(aminomethyl)-6-isobutyl-2-methyl-4-[4-(trifluoromethyl)phenyl]nicotinate (3.3 g, yield 96%) was obtained as a white powder from tert-butyl 5-cyano-6-isobutyl-2-methyl-4-[4-(trifluoromethyl)phenyl]nicotinate (3.5 g, 8.2 mmol) according to a method similar to the method of Example 1-4).

¹H-NMR (CDCl₃) δ:0.99 (6H, d, J=6.6 Hz), 1.17 (9H, s), 1.38 (2H, brs), 2.15-2.35 (1H, m), 2.57 (3H, s), 2.80 (2H, d, J=7.4 Hz), 3.60 (2H, s), 7.42 (2H, d, J=8.0 Hz), 7.70 (2H, d, J=8.0 Hz).

melting point: 88-90° C.

### Example 72

### 5-(aminomethyl)-6-isobutyl-2-methyl-4-[4-(trifluoromethyl)phenyl]nicotinic acid hydrochloride

5-(Aminomethyl)-6-isobutyl-2-methyl-4-[4-(trifluoromethyl)phenyl]nicotinic acid hydrochloride (0.51 g, yield 53%) was obtained as a white powder from tert-butyl 5-(aminomethyl)-6-isobutyl-2-methyl-4-[4-(trifluoromethyl)phenyl]nicotinate (1.0 g, 2.3 mmol) according to a method similar to the method of Example 24.

¹H-NMR (DMSO-d₆) δ:0.97 (6H, d, J=6.6 Hz), 2.15-2.35 (1H, m), 2.51 (3H, s), 2.78 (2H, d, J=7.2 Hz), 3.75 (2H, s), 7.56 (2H, d, J=8.0 Hz), 7.87 (2H, d, J=8.0 Hz), 8.01 (2H, brs).

### Example 73

### tert-butyl 5-(aminomethyl)-6-isobutyl-4-[4-(methoxycarbonyl)phenyl]-2-methylnicotinate

1) Methyl 4-(2-cyano-5-methyl-3-oxohex-1-en-1-yl)benzoate was obtained as a crude product (10.1 g) from 5-methyl-3-oxohexanenitrile (4.0 g, 32 mmol) and methyl 4-formylbenzoate (5.3 g, 32 mmol) according to a method similar to the method of Example 29-1).

2) tert-Butyl 5-cyano-6-isobutyl-4-[4-(methoxycarbonyl)phenyl]-2-methyl-1,4-dihydropyridine-3-carboxylate (5.9 g, yield 45%) was obtained as a white powder from the crude product (10.1 g) obtained in the aforementioned 1) and tert-butyl 3-aminocrotonate (5.25 g, 33 mmol) according to a method similar to the method of Example 1-2). That is, the aforementioned crude product and tert-butyl 3-aminocrotonate were dissolved in methanol (200 mL) and the mixture was heated under reflux for 2 hrs. The reaction mixture was concentrated under reduced pressure and the residue was purified by silica gel column chromatography to give tert-butyl 5-cyano-6-isobutyl-4-[4-(methoxycarbonyl)phenyl]-2-methyl-1,4-dihydropyridine-3-carboxylate.

¹H-NMR (CDCl₃) δ:0.91 (3H, d, J=6.6 Hz), 0.98 (3H, d, J=6.6 Hz), 1.26 (9H, s), 1.75-2.00 (1H, m), 2.15-2.35 (2H, m), 2.36 (3H, s), 3.90 (3H, s), 4.63 (1H, s), 5.69 (1H, brs), 7.32 (2H, d, J=8.3 Hz), 7.99 (2H, d, J=8.3 Hz).

melting point: 191-193° C.

3) tert-Butyl 5-cyano-6-isobutyl-4-[4-(methoxycarbonyl)phenyl]-2-methylnicotinate (5.4 g, yield 95%) was obtained as a white powder from tert-butyl 5-cyano-6-isobutyl-4-[4-(methoxycarbonyl)phenyl]-2-methyl-1,4-dihydropyridine-3-carboxylate (5.7 g, 14 mmol) according to a method similar to the method of Example 23-3).

¹H-NMR (CDCl₃) δ:1.01 (6H, d, J=6.6 Hz), 1.23 (9H, s), 2.20-2.35 (1H, m), 2.67 (3H, s), 2.94 (2H, d, J=7.4 Hz), 3.96 (3H, s), 7.40-7.50 (2H, m), 8.10-8.20 (2H, m).

melting point: 108-109° C.

4) tert-Butyl 5-(aminomethyl)-6-isobutyl-4-[4-(methoxycarbonyl)phenyl]-2-methylnicotinate (5.0 g, yield 94%) was obtained as a white powder from tert-butyl 5-cyano-6-isobutyl-4-[4-(methoxycarbonyl)phenyl]-2-methylnicotinate (5.3 g, 13 mmol) according to a method similar to the method of Example 1-4).

¹H-NMR (CDCl₃) δ:0.99 (6H, d, J=6.6 Hz), 1.17 (9H, s), 1.49 (2H, brs), 2.15-2.35 (1H, m), 2.57 (3H, s), 2.79 (2H, d, J=7.2 Hz), 3.59 (2H, s), 3.96 (3H, s), 7.30-7.40 (2H, m), 8.05-8.15 (2H, m).

melting point: 77-81° C.

### Example 74

### 5-(aminomethyl)-6-isobutyl-4-[4-(methoxycarbonyl)phenyl]-2-methylnicotinic acid hydrochloride

5-(Aminomethyl)-6-isobutyl-4-[4-(methoxycarbonyl)phenyl]-2-methylnicotinic acid hydrochloride (0.50 g, yield 66%) was obtained as a white powder from tert-butyl 5-(aminomethyl)-6-isobutyl-4-[4-(methoxycarbonyl)phenyl]-2-methylnicotinate (0.80 g, 1.9 mmol) according to a method similar to the method of Example 24.

¹H-NMR (DMSO-d₆) δ:0.93 (6H, d, J=6.6 Hz), 2.05-2.25 (1H, m), 2.41 (3H, s), 2.70 (2H, d, J=7.0 Hz), 3.54 (2H, s), 3.88 (3H, s), 7.41 (2H, d, J=8.1 Hz), 7.95 (2H, d, J=8.1 Hz).

### Example 75

### tert-butyl 5-(aminomethyl)-4-(4-ethylphenyl)-6-isobutyl-2-methylnicotinate

1) 3-(4-Ethylphenyl)-2-(3-methylbutanoyl)acrylonitrile was obtained as a crude product (8.8 g) from 5-methyl-3-oxohexanenitrile (4.0 g, 32 mmol) and 4-ethylbenzaldehyde (4.3 g, 32 mmol) according to a method similar to the method of Example 29-1).

2) tert-Butyl 5-cyano-4-(4-ethylphenyl)-6-isobutyl-2-methyl-1,4-dihydropyridine-3-carboxylate (7.8 g, yield 64%) was obtained as a white powder from the crude product (8.8 g) obtained in the aforementioned 1) and tert-butyl 3-aminocrotonate (5.47 g, 35 mmol) according to a method similar to the method of Example 1-2). That is, the aforementioned crude product and tert-butyl 3-aminocrotonate were dissolved in methanol (200 mL) and the mixture was heated under reflux for 4 hrs. The reaction mixture was concentrated under reduced pressure and the residue was purified by silica gel column chromatography to give tert-butyl 5-cyano-4-(4-ethylphenyl)-6-isobutyl-2-methyl-1,4-dihydropyridine-3-carboxylate.

¹H-NMR (CDCl₃) δ:0.94 (3H, d, J=6.5 Hz), 0.99 (3H, d, J=6.5 Hz), 1.20 (3H, t, J=7.6 Hz), 1.28 (9H, s), 1.80-2.00 (1H, m), 2.10-2.30 (2H, m), 2.32 (3H, s), 2.61 (2H, q, J=7.6 Hz), 4.52 (1H, s), 5.55 (1H, brs), 7.10 (2H, d, J=8.3 Hz), 7.14 (2H, d, J=8.3 Hz).

melting point: 165-166° C.

3) tert-Butyl 5-cyano-4-(4-ethylphenyl)-6-isobutyl-2-methylnicotinate (5.2 g, yield 67%) was obtained as a white powder from tert-butyl 5-cyano-4-(4-ethylphenyl)-6-isobutyl-2-methyl-1,4-dihydropyridine-3-carboxylate (7.8 g, 21 mmol) according to a method similar to the method of Example 23-3).

¹H-NMR (CDCl₃) δ:1.01 (6H, d, J=6.6 Hz), 1.23 (9H, s), 1.26 (3H, t, J=7.6 Hz), 2.20-2.35 (1H, m), 2.64 (3H, s), 2.71 (2H, q, J=7.6 Hz), 2.94 (2H, d, J=7.4 Hz), 7.20-7.35 (4H, m).

melting point: 85-86° C.

4) tert-Butyl 5-(aminomethyl)-4-(4-ethylphenyl)-6-isobutyl-2-methylnicotinate (7.0 g, yield 97%) was obtained as a white powder from tert-butyl 5-cyano-4-(4-ethylphenyl)-6-isobutyl-2-methylnicotinate (7.2 g, 19 mmol) according to a method similar to the method of Example 1-4).

¹H-NMR (CDCl₃) δ:0.98 (6H, d, J=6.6 Hz), 1.17 (9H, s), 1.25 (3H, t, J=7.5 Hz), 1.38 (2H, brs), 2.15-2.30 (1H, m), 2.55 (3H, s), 2.69 (2H, q, J=7.5 Hz), 2.78 (2H, d, J=7.4 Hz), 3.63 (2H, s), 7.15 (2H, d, J=7.9 Hz), 7.24 (2H, d, J=7.9 Hz).

melting point: 50-52° C.

### Example 76

### 5-(aminomethyl)-4-(4-ethylphenyl)-6-isobutyl-2-methylnicotinic acid hydrochloride

5-(Aminomethyl)-4-(4-ethylphenyl)-6-isobutyl-2-methylnicotinic acid hydrochloride (0.52 g, yield 79%) was obtained as a white powder from tert-butyl 5-(aminomethyl)-4-(4-ethylphenyl)-6-isobutyl-2-methylnicotinate (0.70 g, 1.8 mmol) according to a method similar to the method of Example 24.

¹H-NMR (DMSO-d₆) δ:0.95 (6H, d, J=7.5 Hz), 1.23 (3H, t, J=7.5 Hz), 2.10-2.30 (1H, m), 2.47 (3H, s), 2.67 (2H, q, J=7.5 Hz), 2.77 (2H, d, J=7.0 Hz), 3.74 (2H, s), 7.22 (2H, d, J=8.0 Hz), 7.30 (2H, d, J=8.0 Hz), 8.81 (1H, brs).

### Example 77

### methyl 5-(aminomethyl)-4-(4-chlorophenyl)-2-ethyl-6-neopentylnicotinate

1) Methyl 3-aminopent-2-enoate was obtained as a crude product (20 g) from methyl 3-oxopentanoate (13 g, 100 mmol) and ammonium acetate (38.5 g, 500 mmol) according to a method similar to the method of Example 12-1).

2) Methyl 4-(4-chlorophenyl)-5-cyano-2-ethyl-6-neopentyl-1,4-dihydropyridine-3-carboxylate (1.4 g, yield 23%) was obtained as a yellow powder from 5,5-dimethyl-3-oxohexanenitrile (5.1 g, 32 mmol), 4-chlorobenzaldehyde (4.5 g, 32 mmol) and the crude product (3.2 g) obtained in the aforementioned 1), according to a method similar to the method of Example 1-2).

¹H-NMR (CDCl₃) δ:0.95-1.05 (3H, m), 1.01 (9H, s), 2.20 (1H, d, J=13.8 Hz), 2.37 (1H, d, J=13.8 Hz), 2.77 (2H, q, J=7.5 Hz), 3.58 (3H, s), 4.60 (1H, s), 5.63 (1H, brs), 7.10-7.20 (2H, m), 7.25-7.30 (2H, m).

3) Methyl 4-(4-chlorophenyl)-5-cyano-2-ethyl-6-neopentylnicotinate (0.58 g, yield 43%) was obtained as a pale-yellow powder from methyl 4-(4-chlorophenyl)-5-cyano-2-ethyl-6-neopentyl-1,4-dihydropyridine-3-carboxylate (1.4 g, 3.7 mmol) according to a method similar to the method of Example 23-3).

¹H-NMR (CDCl₃) δ:1.07 (9H, s), 1.33 (3H, t, J=7.5 Hz), 2.87 (2H, q, J=7.5 Hz), 3.03 (2H, s), 3.61 (3H, s), 7.25-7.35 (2H, m), 7.45-7.50 (2H, m).

melting point: 120-121° C.

4) Methyl 5-(aminomethyl)-4-(4-chlorophenyl)-2-ethyl-6-neopentylnicotinate (0.49 g, yield 85%) was obtained as a pale-yellow oil from methyl 4-(4-chlorophenyl)-5-cyano-2-ethyl-6-neopentylnicotinate (0.57 g, 1.5 mmol) according to a method similar to the method of Example 23-4).

¹H-NMR (CDCl₃) δ:1.03 (9H, s), 1.30 (3H, t, J=7.5 Hz), 1.42 (2H, brs), 2.77 (2H, q, J=7.5 Hz), 2.89 (2H, s), 3.51 (3H, s), 3.69 (2H, s), 7.15-7.25 (2H, m), 7.35-7.45 (2H, m).

### Example 78

### 5-(aminomethyl)-4-(4-chlorophenyl)-2-ethyl-6-neopentylnicotinic acid dihydrochloride

1) Methyl 5-{[(tert-butoxycarbonyl)amino]methyl}-4-(4-chlorophenyl)-2-ethyl-6-neopentylnicotinate (0.52 g, yield 97%) was obtained as a white powder from methyl 5-(aminomethyl)-4-(4-chlorophenyl)-2-ethyl-6-neopentylnicotinate (0.42 g, 1.1 mmol) according to a method similar to the method of Example 2-1).

¹H-NMR (CDCl₃) δ:1.02 (9H, s), 1.30 (3H, t, J=7.5 Hz), 1.38 (9H, s), 2.78 (2H, q, J=7.5 Hz), 2.87 (2H, s), 3.51 (3H, s), 4.18 (3H, brs), 7.10-7.20 (2H, m), 7.30-7.45 (2H, m).

2) 5-{[(tert-Butoxycarbonyl)amino]methyl}-4-(4-chlorophenyl)-2-ethyl-6-neopentylnicotinic acid (0.37 g, yield 81%) was obtained as a white powder from methyl 5-{[(tert-butoxycarbonyl)amino]methyl}-4-(4-chlorophenyl)-2-ethyl-6-neopentylnicotinate (0.47 g, 0.99 mmol) according to a method similar to the method of Example 2-2).

¹H-NMR (CDCl₃) δ:1.01 (9H, s), 1.24 (3H, t, J=7.4 Hz), 1.33 (9H, s), 2.73 (2H, q, J=7.4 Hz), 2.73 (2H, s), 3.92 (2H, d, J=4.5 Hz), 6.96 (1H, t, J=4.5 Hz), 7.25-7.35 (2H, m), 7.47 (2H, d, J=8.3 Hz), 13.05 (1H, brs).

melting point: 71-72° C.

3) 5-(Aminomethyl)-4-(4-chlorophenyl)-2-ethyl-6-neopentylnicotinic acid dihydrochloride (0.24 g, yield 83%) was obtained as a white powder from 5-{[(tert-butoxycarbonyl)amino]methyl}-4-(4-chlorophenyl)-2-ethyl-6-neopentylnicotinic acid (0.30 g, 0.65 mmol) according to a method similar to the method of Example 2-3).

¹H-NMR (DMSO-d₆) δ:1.03 (9H, s), 1.26 (3H, t, J=7.4 Hz), 2.79 (2H, q, J=7.4 Hz), 2.90 (2H, brs), 3.83 (2H, d, J=5.7 Hz), 7.36 (2H, d, J=8.5 Hz), 7.50-7.60 (2H, m), 8.12 (3H, brs).

melting point: 230-235° C.

### Example 79

### tert-butyl 5-(aminomethyl)-4-(4-chlorophenyl)-2-isopropyl-6-neopentylnicotinate

1) tert-Butyl 4-(4-chlorophenyl)-5-cyano-2-isopropyl-6-neopentyl-1,4-dihydropyridine-3-carboxylate (2.00 g, yield 16%) was obtained as a white solid from 5,5-dimethyl-3-oxohexanenitrile (5.67 g, 36.7 mmol), 4-chlorobenzaldehyde (5.16 g, 36.7 mmol) and tert-butyl 3-amino-4-methylpent-2-enoate (5.98 g, 30 mmol) according to a method similar to the method of Example 1-2).

¹H-NMR (CDCl₃) δ:1.02 (9H, s), 1.04 (3H, d, J=6.8 Hz), 1.21 (3H, d, J=7.0 Hz), 1.28 (9H, s), 2.20 (1H, d, J=13.9 Hz), 2.33 (1H, d, J=14.1 Hz), 4.07-4.30 (1H, m), 4.55 (1H, s), 5.65 (1H, s), 7.16 (2H, d, J=8.3 Hz), 7.22-7.35 (2H, m).

2) tert-Butyl 4-(4-chlorophenyl)-5-cyano-2-isopropyl-6-neopentylnicotinate (1.91 g, yield 96%) was obtained as a yellow solid from tert-butyl 4-(4-chlorophenyl)-5-cyano-2-isopropyl-6-neopentyl-1,4-dihydropyridine-3-carboxylate (2.00 g, 4.66 mmol) according to a method similar to the method of Example 23-3).

¹H-NMR (CDCl₃) δ:1.06 (9H, s), 1.27 (9H, s), 1.32 (6H, d, J=6.6 Hz), 3.00 (2H, s), 3.13-3.25 (1H, m), 7.32 (2H, d, J=8.5 Hz), 7.45 (2H, d, J=8.5 Hz).

3) tert-Butyl 5-(aminomethyl)-4-(4-chlorophenyl)-2-isopropyl-6-neopentylnicotinate (1.24 g, yield 67%) was obtained as a white solid from tert-butyl 4-(4-chlorophenyl)-5-cyano-2-isopropyl-6-neopentylnicotinate (1.80 g, 4.27 mmol) according to a method similar to the method of Example 23-4).

¹H-NMR (CDCl₃) δ:1.04 (9H, s), 1.21 (9H, s), 1.30 (6H, d, J=6.6 Hz), 2.85 (2H, s), 3.01-3.16 (1H, m), 3.64 (2H, s), 7.22 (2H, d, J=8.5 Hz), 7.40 (2H, d, J=8.5 Hz).

### Example 80

### 5-(aminomethyl)-4-(4-chlorophenyl)-2-isopropyl-6-neopentylnicotinic acid dihydrochloride

5-(Aminomethyl)-4-(4-chlorophenyl)-2-isopropyl-6-neopentylnicotinic acid dihydrochloride (393 mg, yield 93%) was obtained as a yellow solid from tert-butyl 5-(aminomethyl)-4-(4-chlorophenyl)-2-isopropyl-6-neopentylnicotinate (406 mg, 0.941 mmol) according to a method similar to the method of Example 24-1).

¹H-NMR (DMSO-d₆) δ:1.04 (9H, s), 1.25 (6H, d, J=6.8 Hz), 2.88 (2H, s), 3.05-3.14 (1H, m), 3.81 (2H, d, J=5.3 Hz), 7.36 (2H, d, J=8.5 Hz), 7.55 (2H, d, J=8.5 Hz), 8.11 (3H, brs).

### Example 81

### tert-butyl 5-(aminomethyl)-4-(4-chlorophenyl)-6-isobutyl-2-isopropylnicotinate

1) tert-Butyl 4-(4-chlorophenyl)-5-cyano-6-isobutyl-2-isopropyl-1,4-dihydropyridine-3-carboxylate (6.18 g, yield 50%) was obtained as a yellow solid from 5-methyl-3-oxohexanenitrile (4.14 g, 33 mmol), 4-chlorobenzaldehyde (4.64 g, 33 mmol) and tert-butyl 3-amino-4-methylpent-2-enoate (5.98 g, 30 mmol) according to a method similar to the method of Example 1-2).

¹H-NMR (CDCl₃) δ:0.97 (6H, dd, J=8.5, 6.8 Hz), 1.14 (3H, d, J=7.0 Hz), 1.22 (3H, d, J=7.0 Hz), 1.28 (9H, s), 1.81-1.98 (1H, m), 2.25 (2H, d, J=7.4 Hz), 4.09-4.26 (1H, m), 4.55 (1H, s), 5.71 (1H, s), 7.15 (2H, d, J=8.3 Hz), 7.25-7.27 (2H, m).

2) tert-Butyl 4-(4-chlorophenyl)-5-cyano-6-isobutyl-2-isopropylnicotinate (6.10 g, yield 99%) was obtained as a yellow oil from tert-butyl 4-(4-chlorophenyl)-5-cyano-6-isobutyl-2-isopropyl-1,4-dihydropyridine-3-carboxylate (6.16 g, 14.8 mmol) according to a method similar to the method of Example 23-3).

¹H-NMR (CDCl₃) δ:1.01 (6H, d, J=6.6 Hz), 1.26 (9H, s), 1.32 (6H, d, J=6.8 Hz), 2.22-2.39 (1H, m), 2.95 (2H, d, J=7.2 Hz), 3.19-3.25 (1H, m), 7.33 (2H, d, J=8.7 Hz), 7.46 (2H, d, J=8.7 Hz).

3) tert-Butyl 5-(aminomethyl)-4-(4-chlorophenyl)-6-isobutyl-2-isopropylnicotinate (5.52 g, yield 89%) was obtained as a white solid from tert-butyl 4-(4-chlorophenyl)-5-cyano-6-isobutyl-2-isopropylnicotinate (6.10 g, 1.48 mmol) according to a method similar to the method of Example 23-4).

¹H-NMR (CDCl₃) δ:0.99 (6H, d, J=6.8 Hz), 1.21 (9H, s), 1.30 (6H, d, J=6.8 Hz), 2.23-2.39 (1H, m), 2.78 (2H, d, J=7.2 Hz), 3.01-3.16 (1H, m), 3.59 (1H, s), 7.22 (2H, d, J=8.5 Hz), 7.39 (2H, d, J=8.5 Hz).

### Example 82

### 5-(aminomethyl)-4-(4-chlorophenyl)-6-isobutyl-2-isopropylnicotinic acid dihydrochloride

5-(Aminomethyl)-4-(4-chlorophenyl)-6-isobutyl-2-isopropylnicotinic acid dihydrochloride (263 mg, yield 62%) was obtained as a yellow solid from tert-butyl 5-(aminomethyl)-4-(4-chlorophenyl)-6-isobutyl-2-isopropylnicotinate (404 mg, 0.969 mmol) according to a method similar to the method of Example 24-1).

¹H-NMR (DMSO-d₆) δ:0.99 (6H, d, J=6.6 Hz), 1.25 (6H, d, J=6.8 Hz), 2.20-2.39 (1H, m), 2.83 (2H, d, J=7.0 Hz), 3.01-3.19 (1H, m), 3.77 (2H, d, J=5.3 Hz), 7.36 (2H, d, 8.5 Hz), 7.55 (2H, d, J=8.3 Hz), 8.14 (3H, brs).

### Example 83

### tert-butyl 5-(aminomethyl)-4-(4-chlorophenyl)-2,6-diisobutylnicotinate

1) tert-Butyl 3-amino-5-methylhex-2-enoate was obtained as a crude product (20.2 g) from Meldrum's acid (17.3 g, 120 mmol) and isovaleryl chloride (15.8 mL, 132 mmol) according to a method similar to the method of Example 25-1).

2) tert-Butyl 4-(4-chlorophenyl)-5-cyano-2,6-diisobutyl-1,4-dihydropyridine-3-carboxylate (10.2 g, yield 72%) was obtained as a pale-yellow powder from 5-methyl-3-oxohexanenitrile (4.1 g, 33 mmol), 4-chlorobenzaldehyde (4.6 g, 33 mmol) and the crude product (10.1 g) obtained in the aforementioned 1), according to a method similar to the method of Example 1-2).

¹H-NMR (CDCl₃) δ:0.95-1.05 (12H, m), 1.29 (9H, s), 1.80-2.05 (2H, m), 2.15-2.35 (2H, m), 2.55-2.70 (2H, m), 4.60 (1H, s), 5.51 (1H, brs), 7.15-7.25 (2H, m), 7.25-7.30 (2H, m).

melting point: 166-168° C.

3) tert-Butyl 4-(4-chlorophenyl)-5-cyano-2,6-diisobutylnicotinate (9.6 g, yield 99%) was obtained as a white powder from tert-butyl 4-(4-chlorophenyl)-5-cyano-2,6-diisobutyl-1,4-dihydropyridine-3-carboxylate (9.8 g, 23 mmol) according to a method similar to the method of Example 23-3).

¹H-NMR (CDCl₃) δ:0.95 (6H, d, J=6.8 Hz), 1.00 (6H, d, J=6.6 Hz), 1.25 (9H, s), 2.15-2.40 (2H, m), 2.76 (2H, d, J=7.2 Hz), 2.95 (2H, d, J=7.4 Hz), 7.30-7.35 (2H, m), 7.40-7.50 (2H, m). 4) tert-Butyl 5-(aminomethyl)-4-(4-chlorophenyl)-2,6-diisobutylnicotinate (0.97 g, yield 96%) was obtained as a white powder from tert-butyl 4-(4-chlorophenyl)-5-cyano-2,6-diisobutylnicotinate (1.0 g, 2.3 mmol) according to a method similar to the method of Example 23-4).

¹H-NMR (CDCl₃) δ:0.94 (6H, d, J=6.6 Hz), 0.98 (6H, d, J=6.6 Hz), 1.20 (9H, s), 1.48 (2H, brs), 2.15-2.35 (2H, m), 2.67 (2H, d, J=7.4 Hz), 2.80 (2H, d, J=7.4 Hz), 3.61 (2H, s), 7.20-7.25 (2H, m), 7.35-7.45 (2H, m).

### Example 84

### 5-(aminomethyl)-4-(4-chlorophenyl)-2,6-diisobutylnicotinic acid dihydrochloride

5-(Aminomethyl)-4-(4-chlorophenyl)-2,6-diisobutylnicotinic acid dihydrochloride (0.92 g, yield 98%) was obtained as a white powder from tert-butyl 5-(aminomethyl)-4-(4-chlorophenyl)-2,6-diisobutylnicotinate (0.90 g, 2.1 mmol) according to a method similar to the method of Example 24-1).

¹H-NMR (DMSO-d₆) δ:0.90 (6H, d, J=6.6 Hz), 0.97 (6H, d, J=6.6 Hz), 2.10-2.35 (2H, m), 2.66 (2H, d, J=6.4 Hz), 2.84 (2H, d, J=6.2 Hz), 3.79 (2H, d, J=5.5 Hz), 7.36 (2H, d, J=8.5 Hz), 7.50-7.60 (2H, m), 8.17 (3H, brs).

melting point: 205° C. (dec.)

### Example 85

### tert-butyl 5-(aminomethyl)-4-(4-chlorophenyl)-2-isobutyl-6-neopentylnicotinate

1) tert-Butyl 4-(4-chlorophenyl)-5-cyano-2-isobutyl-6-neopentyl-1,4-dihydropyridine-3-carboxylate was obtained as a crude product (7.9 g) from 5,5-dimethyl-3-oxohexanenitrile (4.6 g, 33 mmol), 4-chlorobenzaldehyde (4.6 g, 33 mmol) and the crude product (10.1 g) of tert-butyl 3-amino-5-methylhex-2-enoate obtained in Example 83-1), according to a method similar to the method of Example 1-2).

2) tert-Butyl 4-(4-chlorophenyl)-5-cyano-2-isobutyl-6-neopentylnicotinate (5.5 g, yield 37%) was obtained as a white powder from the crude product (7.9 g) obtained in the aforementioned 1) according to a method similar to the method of Example 23-3).

¹H-NMR (CDCl₃) δ:0.95 (6H, d, J=6.6 Hz), 1.06 (9H, s), 1.26 (9H, s), 2.20-2.35 (1H, m), 2.76 (2H, d, J=7.2 Hz), 3.01 (2H, s), 7.30-7.35 (2H, m), 7.40-7.50 (2H, m).

3) tert-Butyl 5-(aminomethyl)-4-(4-chlorophenyl)-2-isobutyl-6-neopentylnicotinate (4.5 g, yield 86%) was obtained as a yellow powder from tert-butyl 4-(4-chlorophenyl)-5-cyano-2-isobutyl-6-neopentylnicotinate (5.2 g, 12 mmol) according to a method similar to the method of Example 23-4).

¹H-NMR (CDCl₃) δ:0.93 (6H, d, J=6.8 Hz), 1.02 (9H, s), 1.20 (9H, s), 1.86 (2H, brs), 2.15-2.35 (1H, m), 2.67 (2H, d, J=7.4 Hz), 2.87 (2H, s), 3.71 (2H, s), 7.20-7.25 (2H, m), 7.35-7.45 (2H, m).

### Example 86

### 5-(aminomethyl)-4-(4-chlorophenyl)-2-isobutyl-6-neopentylnicotinic acid dihydrochloride

5-(Aminomethyl)-4-(4-chlorophenyl)-2-isobutyl-6-neopentylnicotinic acid dihydrochloride (0.29 g, yield 56%) was obtained as a white powder from tert-butyl 5-(aminomethyl)-4-(4-chlorophenyl)-2-isobutyl-6-neopentylnicotinate (0.50 g, 1.1 mmol) according to a method similar to the method of Example 24-1).

¹H-NMR (DMSO-d₆) δ:0.90 (6H, d, J=6.6 Hz), 1.02 (9H, s), 2.15-2.30 (1H, m), 2.66 (2H, q, J=7.2 Hz), 2.91 (2H, s), 3.84 (2H, d, J=5.5 Hz), 7.30-7.40 (2H, m), 7.50-7.60 (2H, m), 8.12 (3H, brs).

melting point: 251° C. (dec.)

### Example 87

### [5-(aminomethyl)-2-methyl-4-(4-methylphenyl)-6-neopentylpyridin-3-yl]acetonitrile dihydrochloride

1) tert-Butyl {[5-(hydroxymethyl)-6-methyl-4-(4-methylphenyl)-2-neopentylpyridin-3-yl]methyl}carbamate (4.5 g, yield 48%) was obtained as a white powder from methyl 5-{[(tert-butoxycarbonyl)amino]methyl}-2-methyl-4-(4-methylphenyl)-6-neopentylnicotinate (10 g, 22.7 mmol) according to a method similar to the method of Example 5-1).

¹H-NMR (CDCl₃) δ:1.01 (9H, s), 1.37 (9H, s), 2.41 (3H, s), 2.67 (3H, s), 2.84 (2H, s), 4.10 (2H, d, J=4.9 Hz), 4.16 (1H, s), 4.36 (2H, d, J=5.7 Hz), 7.05 (2H, d, J=8.1 Hz), 7.26 (2H, d, J=8.1 Hz).

2) A mixture of tert-butyl {[5-(hydroxymethyl)-6-methyl-4-(4-methylphenyl)-2-neopentylpyridin-3-yl]methyl}carbamate (0.9 g, 2.2 mmol), triethylamine (0.4 g, 4.0 mmol) and tetrahydrofuran (30 mL) was cooled to 0° C. and methanesulfonyl chloride (0.3 g, 2.6 mmol) was added dropwise. After stirring at room temperature for 30 min., the reaction mixture was poured into saturated aqueous sodium hydrogen carbonate. The mixture was extracted with ethyl acetate and the extract was dried over anhydrous magnesium sulfate. The solvent was evaporated under reduced pressure to give [5-{[(tert-butoxycarbonyl)amino]methyl}-2-methyl-4-(4-methylphenyl)-6-neopentylpyridin-3-yl]methyl methanesulfonate (0.85 g, yield 79%) as a white powder.

¹H-NMR (CDCl₃) δ:1.01 (9H, s), 1.37 (9H, s), 2.41 (3H, s), 2.67 (3H, s), 2.75 (3H, s), 2.86 (2H, s), 4.11 (2H, d, J=4.9 Hz), 4.17 (1H, s), 4.91 (2H, s), 7.04 (2H, d, J=8.1 Hz), 7.27 (2H, d, J=8.1 Hz).

3) [5-{[(tert-Butoxycarbonyl)amino]methyl}-2-methyl-4-(4-methylphenyl)-6-neopentylpyridin-3-yl]methyl methanesulfonate (0.84 g, 1.7 mmol) was dissolved in dimethyl sulfoxide (10 mL) and potassium cyanide (0.14 g, 2.0 mmol) was added. The mixture was stirred at 60° C. for 1 hr. Ethyl acetate was added to the reaction mixture, and the mixture was washed successively with water and saturated brine and dried over anhydrous magnesium sulfate. The solvent was evaporated under reduced pressure. The residue was purified by silica gel column chromatography to give tert-butyl {[5-(cyanomethyl)-6-methyl-4-(4-methylphenyl)-2-neopentylpyridin-3-yl]methyl}carbamate (0.45 g, yield 63%) as a powder.

¹H-NMR (CDCl₃) δ:1.01 (9H, s), 1.37 (9H, s), 2.43 (3H, s), 2.65 (3H, s), 2.85 (2H, s), 3.30 (2H, s), 4.11 (2H, d, J=4.5 Hz), 4.17 (1H, s), 7.05 (2H, d, J=8.0 Hz), 7.30 (2H, d, J=8.0 Hz).

4) [5-(Aminomethyl)-2-methyl-4-(4-methylphenyl)-6-neopentylpyridin-3-yl]acetonitrile dihydrochloride (0.28 g, 76%) was obtained as a powder from tert-butyl {(5-(cyanomethyl)-6-methyl-4-(4-methylphenyl)-2-neopentylpyridin-3-yl]methyl}carbamate (0.4 g, 0.95 mmol) according to a method similar to the method of Example 2-3).

¹H-NMR (DMSO-d₆) δ:1.01 (9H, s), 2.42 (3H, s), 2.76 (3H, s), 3.06 (2H, s), 3.59 (2H, s), 3.80 (2H, d, J=5.3 Hz), 7.24 (2H, d, J=7.9 Hz), 7.42 (2H, d, J=7.9 Hz), 8.20 (3H, s).

### Example 88

### 2-[5-(aminomethyl)-2-methyl-4-(4-methylphenyl)-6-neopentylpyridin-3-yl]acetamide dihydrochloride

1) tert-Butyl {[5-(2-amino-2-oxoethyl)-6-methyl-4-(4-methylphenyl)-2-neopentylpyridin-3-yl]methyl}carbamate (0.3 g, 82%) was obtained as a powder from tert-butyl {[5-(cyanomethyl)-6-methyl-4-(4-methylphenyl)-2-neopentylpyridin-3-yl]methyl}carbamate (0.35 g, 0.83 mmol) according to a method similar to the method of Example 6-1).

¹H-NMR (CDCl₃) δ:1.02 (9H, s), 1.37 (9H, s), 2.40 (3H, s), 2.56 (3H, s), 2.84 (2H, s), 3.30 (2H, s), 4.10 (2H, d, J=4.9 Hz), 4.19 (1H, s), 5.15 (1H, s), 5.20 (1H, s), 7.00 (2H, d, J=7.9 Hz), 7.24 (2H, d, J=7.9 Hz).

2) 2-[5-(Aminomethyl)-2-methyl-4-(4-methylphenyl)-6-neopentylpyridin-3-yl]acetamide dihydrochloride (0.18 g, 85%) was obtained as a powder from tert-butyl {[5-(2-amino-2-oxoethyl)-6-methyl-4-(4-methylphenyl)-2-neopentylpyridin-3-yl]methyl}carbamate (0.22 g, 0.5 mmol) according to a method similar to the method of Example 6-2).

¹H-NMR (DMSO-d₆) δ:1.03 (9H, s), 2.41 (3H, s), 2.77 (2H, s), 3.29 (3H, s), 3.87 (2H, s), 4.28 (2H, s), 7.03 (1H, s), 7.20 (2H, d, J=7.8 Hz), 7.38 (2H, d, J=7.8 Hz), 7.39 (1H, s), 8.24 (3H, s).

### Example 89

### [5-(aminomethyl)-2-methyl-4-(4-methylphenyl)-6-neopentylpyridin-3-yl]methyl acetate dihydrochloride

1) A mixture of tert-butyl {[5-(hydroxymethyl)-6-methyl-4-(4-methylphenyl)-2-neopentylpyridin-3-yl]methyl}carbamate (0.3 g, 0.73 mmol), triethylamine (0.1 g, 1.0 mmol) and tetrahydrofuran (20 mL) was cooled to 0° C. and acetyl chloride (0.06 g, 0.8 mmol) was added dropwise. After stirring at room temperature for 30 min., the reaction mixture was poured into saturated aqueous sodium hydrogen carbonate. The mixture was extracted with ethyl acetate and the extract was dried over anhydrous magnesium sulfate. The solvent was evaporated under reduced pressure to give [5-{[(tert-butoxycarbonyl)amino]methyl}-2-methyl-4-(4-methylphenyl)-6-neopentylpyridin-3-yl]methyl acetate (0.26 g, yield 76%) as a white powder.

¹H-NMR (CDCl₃) δ:1.02 (9H, s), 1.37 (9H, s), 2.00 (3H, s), 2.40 (3H, s), 2.57 (3H, s), 2.85 (2H, s), 4.11 (2H, d, J=4.9 Hz), 4.17 (1H, s), 4.76 (2H, s), 7.00 (2H, d, J=8.1 Hz), 7.22 (2H, d, J=8.1 Hz).

2) [5-(Aminomethyl)-2-methyl-4-(4-methylphenyl)-6-neopentylpyridin-3-yl]methyl acetate dihydrochloride (99 mg, 90%) was obtained as a powder from [5-{[(tert-butoxycarbonyl)amino]methyl}-2-methyl-4-(4-methylphenyl)-6-neopentylpyridin-3-yl]methyl acetate (0.12 g, 0.26 mmol) according to a method similar to the method of Example 2-3).

¹H-NMR (DMSO-d₆) δ:1.02 (9H, s), 1.96 (3H, s), 2.40 (3H, s), 2.78 (3H, s), 3.14 (2H, s), 3.82 (2H, s), 4.72 (2H, s), 7.21 (2H, d, J=7.8 Hz), 7.36 (2H, d, J=7.8 Hz), 8.23 (3H, s).

### Example 90

### {[2-isobutyl-6-methyl-4-(4-methylphenyl)-5-({[4-(methylthio)phenyl]thio}methyl)pyridin-3-yl]methyl}amine dihydrochloride

1) A mixture of tert-butyl {[5-(hydroxymethyl)-2-isobutyl-6-methyl-4-(4-methylphenyl)pyridin-3-yl]methyl}carbamate (3.06 g, 7.68 mmol), triethylamine (1.8 mL, 12.9 mmol) and tetrahydrofuran (30 mL) was cooled to 0° C., and methanesulfonyl chloride (0.89 mL, 11.5 mmol) was added dropwise. After stirring at room temperature for 30 min., the reaction mixture was poured into saturated aqueous sodium hydrogen carbonate, and the mixture was extracted with ethyl acetate. The extract was dried over anhydrous magnesium sulfate and the solvent was evaporated under reduced pressure to give [5-{[(tert-butoxycarbonyl)amino]methyl}-6-isobutyl-2-methyl-4-(4-methylphenyl)pyridin-3-yl]methyl methanesulfonate as a crude product. The crude product was dissolved in N,N-dimethylformamide (30 mL). Potassium carbonate (1.77 g, 12.8 mmol) and 4-(methylthio)benzenethiol (1.00 g, 6.40 mmol) were added and the mixture was stirred with heating at 50° C. for 1 hr. The reaction mixture was diluted with ethyl acetate (100 mL) and washed with saturated brine. The organic layer was dried over anhydrous magnesium sulfate and the solvent was evaporated under reduced pressure. The obtained residue was purified by silica gel column chromatography to give tert-butyl {[2-isobutyl-6-methyl-4-(4-methylphenyl)-5-({[4-(methylthio)phenyl]thio}methyl)pyridin-3-yl]methyl}carbamate (3.43 g, yield 99%) as a yellow solid.

¹H-NMR (CDCl₃) δ:0.97 (6H, d, J=6.6 Hz), 1.38 (9H, s), 2.15-2.24 (1H, m), 2.40 (3H, s), 2.45 (3H, s), 2.63 (3H, s), 2.75 (2H, d, J=7.4 Hz), 3.75 (2H, s), 4.02 (2H, d, J=5.1 Hz), 4.18 (1H, brs), 6.98 (2H, d, J=8.1 Hz), 7.03 (2H, d, J=8.7 Hz), 7.08 (2H, d, J=8.7 Hz), 7.20 (2H, d, J=7.9 Hz).

2) {[2-Isobutyl-6-methyl-4-(4-methylphenyl)-5-({[4-(methylthio)phenyl]thio}methyl)pyridin-3-yl]methyl}amine dihydrochloride (380 mg, yield 79%) was obtained as a yellow solid from tert-butyl {[2-isobutyl-6-methyl-4-(4-methylphenyl)-5-({[4-(methylthio)phenyl]thio}methyl)pyridin-3-yl]methyl}carbamate (508 mg, 0.947 mmol) according to a method similar to the method of Example 2-3).

¹H-NMR (DMSO-d₆) δ:0-98 (6H, d, J=6.6 Hz), 2.13-2.22 (1H, m), 2.40 (3H, s), 2.46 (3H, s), 2.78 (3H, s), 3.11 (2H, brs), 3.76 (2H, d, J=4.5 Hz), 3.87 (2H, s), 7.12 (2H, d, J=8.7 Hz), 7.16 (2H, d, J=8.7 Hz), 7.22 (2H, d, J=7.9 Hz), 7.33 (2H, d, J=7.9 Hz), 8.38 (3H, brs).

### Example 91

### {[2-isobutyl-6-methyl-4-(4-methylphenyl)-5-({[4-(methylsulfonyl)phenyl]sulfonyl}methyl)pyridin-3-yl]methyl}amine dihydrochloride

1) To a solution of tert-butyl {[2-isobutyl-6-methyl-4-(4-methylphenyl)-5-({[4-(methylthio)phenyl]thio}methyl)pyridin-3-yl]methyl}carbamate (1.10 g, 2.05 mmol) in methanol (15 mL), water (1.5 mL) and tetrahydrofuran (1.5 mL) were added sulfuric acid (121 mg, 1.23 mmol) and Oxone (trademark, 3.78 g, 6.15 mmol) and the mixture was stirred at room temperature for 2 hrs. The reaction mixture was diluted with ethyl acetate (100 mL) and washed successively with saturated aqueous sodium hydrogen carbonate and saturated brine. The organic layer was dried over anhydrous magnesium sulfate and the solvent was evaporated under reduced pressure. The obtained white solid was washed with diisopropyl ether to give tert-butyl {[2-isobutyl-6-methyl-4-(4-methylphenyl)-5-({[4-(methylsulfonyl)phenyl]sulfonyl}methyl)pyridin-3-yl]methyl}carbamate (1.06 g, yield 86%) as a white powder.

¹H-NMR (CDCl₃) δ:0.98 (6H, d, J=6.8 Hz), 1.38 (9H, s), 2.17-2.27 (1H, m), 2.42 (3H, s), 2.70 (3H, s), 2.78 (2H, d, J=7.2 Hz), 3.09 (3H, s), 4.00 (2H, d, J=5.1 Hz), 4.19 (1H, brs), 4.36 (2H, s), 6.87 (2H, d, J=7.9 Hz), 7.19 (2H, d, J=7.9 Hz), 7.69 (2H, d, J=8.3 Hz), 8.00 (2H, d, J=8.5 Hz).

2) {[2-Isobutyl-6-methyl-4-(4-methylphenyl)-5-({[4-(methylsulfonyl)phenyl]sulfonyl}methyl)pyridin-3-yl]methyl}amine dihydrochloride (480 mg, yield 98%) was obtained as a white powder from tert-butyl {[2-isobutyl-6-methyl-4-(4-methylphenyl)-5-({[4-(methylsulfonyl)phenyl]sulfonyl}methyl)pyridin-3-yl]methyl}carbamate (511 mg, 0.851 mmol) according to a method similar to the method of Example 2-3).

¹H-NMR (DMSO-d₆) δ:0.97 (6H, d, J=6.6 Hz), 2.17-2.27 (1H, m), 2.38 (3H, s), 2.81 (3H, brs), 3.00 (2H, brs), 3.34 (3H, s), 3.68 (2H, brs), 7.03 (2H, d, J=7.4 Hz), 7.22 (2H, d, J=7.9 Hz), 7.77 (2H, d, J=7.0 Hz), 8.11 (2H, d, J=8.5 Hz), 8.26 (3H, brs).

### Example 92

### (6-methyl-4-(4-methylphenyl)-5-{[(4-methyl-4H-1,2,4-triazol-3-yl)thio]methyl}-2-neopentylpyridin-3-yl)methylamine dihydrochloride

1) tert-Butyl [(6-methyl-4-(4-methylphenyl)-5-{[(4-methyl-4H-1,2,4-triazol-3-yl)thio]methyl}-2-neopentylpyridin-3-yl)methyl]carbamate (0.28 g, 77%) was obtained as a powder from [5-{[(tert-butoxycarbonyl)amino]methyl}-2-methyl-4-(4-methylphenyl)-6-neopentylpyridin-3-yl]methyl methanesulfonate (0.35 g, 0.71 mmol) and 4-methyl-4H-1,2,4-triazole-3-thiol (99 mg, 0.86 mmol) according to a method similar to the method of Example 33-1).

¹H-NMR (CDCl₃) δ:1.02 (9H, s), 1.37 (9H, s), 2.39 (3H, s), 2.65 (3H, s), 2.84 (2H, s), 3.41 (3H, s), 4.07 (2H, d, J=5.3 Hz), 4.17 (3H, s), 7.02 (2H, d, J=7.9 Hz), 7.22 (2H, d, J=7.9 Hz), 8.08 (1H, s).

2) (6-Methyl-4-(4-methylphenyl)-5-{[(4-methyl-4H-1,2,4-triazol-3-yl)thio]methyl}-2-neopentylpyridin-3-yl)methylamine dihydrochloride (0.12 g, 72%) was obtained as a powder from tert-butyl [(6-methyl-4-(4-methylphenyl)-5-{[(4-methyl-4H-1,2,4-triazol-3-yl)thio]methyl}-2-neopentylpyridin-3-yl)methyl]carbamate (0.18 g, 0.35 mmol) according to a method similar to the method of Example 2-3).

¹H-NMR (DMSO-d₆) δ:1.02 (9H, s), 2.39 (3H, s), 2.80 (3H, s), 3.19 (2H, s), 3.41 (3H, s), 3.79 (2H, s), 4.05 (2H, s), 7.13 (2H, d, J=8.1 Hz), 7.35 (2H, d, J=8.1 Hz), 8.25 (3H, s), 8.74 (1H, s).

### Example 93

### {6-methyl-4-(4-methylphenyl)-2-neopentyl-5-[(1,3-thiazol-2-ylthio)methyl]pyridin-3-yl}methylamine dihydrochloride

1) tert-Butyl ({6-methyl-4-(4-methylphenyl)-2-neopentyl-5-[(1,3-thiazol-2-ylthio)methyl]pyridin-3-yl}methyl)carbamate (0.25 g, 69%) was obtained as a powder from [5-{[(tert-butoxycarbonyl)amino]methyl}-2-methyl-4-(4-methylphenyl)-6-neopentylpyridin-3-yl]methyl methanesulfonate (0.35 g, 0.71 mmol) and 2-mercaptothiazole (100 mg, 0.86 mmol) according to a method similar to the method of Example 33-1).

¹H-NMR (CDCl₃) δ:1.02 (9H, s), 1.37 (9H, s), 2.38 (3H, s), 2.64 (3H, s), 2.84 (2H, s), 4.08 (2H, d, J=5.1 Hz), 4.17 (3H, s), 7.03 (2H, d, J=7.9 Hz), 7.18 (1H, d, J=3.4 Hz), 7.20 (2H, d, J=7.9 Hz), 7.60 (1H, d, J=3.4 Hz).

2) {6-Methyl-4-(4-methylphenyl)-2-neopentyl-5-[(1,3-thiazol-2-ylthio)methyl]pyridin-3-yl}methylamine dihydrochloride (0.11 g, 80%) was obtained as a powder from tert-butyl ({6-methyl-4-(4-methylphenyl)-2-neopentyl-5-[(1,3-thiazol-2-ylthio)methyl]pyridin-3-yl}methyl)carbamate (0.15 g, 0.29 mmol) according to a method similar to the method of Example 2-3).

¹H-NMR (DMSO-d₆) δ:1.01 (9H, s), 2.38 (3H, s), 2.78 (3H, s), 3.10 (2H, s), 3.78 (2H, s), 4.20 (2H, s), 7.20 (2H, d, J=8.1 Hz), 7.33 (2H, d, J=8.1 Hz), 7.69 (1H, d, J=3.4 Hz), 7.71 (1H, d, J=3.4 Hz), 8.17 (3H, s).

### Example 94

### 5-(aminomethyl)-6-isobutyl-2-methyl-4-(4-methylphenyl)nicotinonitrile dihydrochloride

1) To a solution (20 mL) of tert-butyl {[5-(aminocarbonyl)-2-isobutyl-6-methyl-4-(4-methylphenyl)pyridin-3-yl]methyl}carbamate (1750 mg, 4.2 mmol) in dichloromethane was added triethylamine (1.2 mL, 8.4 mmol), and trifluoromethanesulfonic anhydride (780 μL, 8.4 mmol) was added dropwise under ice-cooling. The mixture was stirred for 30 min. and the reaction mixture was washed successively with water and saturated brine. The organic layer was dried over anhydrous magnesium sulfate and the solvent was evaporated under reduced pressure. The obtained residue was purified by silica gel column chromatography to give tert-butyl {[5-cyano-2-isobutyl-6-methyl-4-(4-methylphenyl)pyridin-3-yl]methyl}carbamate (1130 mg, yield 68%) as white crystals.

¹H-NMR (CDCl₃) δ:0.97 (6H, d, J=6.6 Hz), 1.40 (9H, s), 2.20-2.29 (1H, m), 2.43 (3H, s), 2.77 (3H, s), 2.83 (2H, d, J=9.0 Hz), 4.18 (2H, s), 4.20 (1H, brs), 7.13 (2H, d, J=6.0 Hz), 7.31 (2H, d, J=6.0 Hz).

2) 5-(Aminomethyl)-6-isobutyl-2-methyl-4-(4-methylphenyl)nicotinonitrile dihydrochloride (81 mg, yield 88%) was obtained as a white powder from tert-butyl {[5-cyano-2-isobutyl-6-methyl-4-(4-methylphenyl)pyridin-3-yl]methyl}carbamate (100 mg, 0.25 mmol) according to a method similar to the method of

### Example 2-3).

¹H-NMR (DMSO-d₆) δ:0.95 (6H, d, J=6.6 Hz), 2.21-2.27(1H, m), 2.42 (3H, s), 2.71 (3H, s), 2.89 (2H, d, J=6.9 Hz), 3.82 (2H, d, J=5.4 Hz), 7.33-7.40 (4H, m), 8.50 (3H, brs).

### Example 95

### N-[5-(aminomethyl)-6-isobutyl-2-methyl-4-(4-methylphenyl)pyridin-3-yl]urea dihydrochloride

1) To a solution (3 mL) of 5-{[(tert-butoxycarbonyl)amino]methyl}-6-isobutyl-2-methyl-4-(4-methylphenyl)nicotinic acid (412 mg, 1.0 mmol) in N,N-dimethylformamide was added triethylamine (170 μl, 1.5 mmol), and diphenylphosphoryl azide (260 μL, 1.5 mmol) was added dropwise under ice-cooling. The mixture was stirred for 30 min. and water was added to the reaction mixture. The mixture was extracted with ethyl acetate, and the organic layer was washed with saturated brine and dried over anhydrous magnesium sulfate. The solvent was evaporated under reduced pressure and the obtained residue was dissolved in toluene (3 mL). The mixture was heated under reflux with stirring for 1 hr. 25% Aqueous ammonia (3 mL) was added to the reaction mixture and the mixture was stirred at 100° C. for 1 hr. Water was added to the reaction mixture, and the mixture was extracted with ethyl acetate. The organic layer was washed with saturated brine and dried over anhydrous magnesium sulfate. The solvent was evaporated under reduced pressure and the obtained residue was purified by silica gel column chromatography to give tert-butyl {[5-[(aminocarbonyl)amino]-2-isobutyl-6-methyl-4-(4-methylphenyl)pyridin-3-yl]methyl}carbamate (101 mg, yield 24%) as white crystals.

¹H-NMR (CDCl₃) δ:0.98 (6H, d, J=6.6 Hz), 1.39 (9H, s), 2.15-2.26 (1H, m), 2.39 (3H, s), 2.56 (3H, s), 2.76 (2H, d, J=7.2 Hz), 4.10 (2H, d, J=5.1 Hz), 4.24 (1H, brs), 4.38 (2H, s), 5.50 (1H, s), 7.01 (2H, d, J=7.5 Hz), 7.24 (2H, d, J=7.5 Hz).

2) N-[5-(Aminomethyl)-6-isobutyl-2-methyl-4-(4-methylphenyl)pyridin-3-yl]urea dihydrochloride (84 mg, yield 92%) was obtained as a white powder from tert-butyl {[5-[(aminocarbonyl)amino]-2-isobutyl-6-methyl-4-(4-methylphenyl)pyridin-3-yl]methyl}carbamate (100 mg, 0.23 mmol) according to a method similar to the method of Example 2-3).

¹H-NMR (DMSO-d₆) δ:0.98 (6H, d, J=5.4 Hz), 2.14-2.19 (1H, m), 2.40 (3H, s), 2.53 (3H, s), 3.0. (2H, brs), 3.80 (2H, brs), 3.83 (1H, brs), 5.94 (1H, brs), 7.20 (2H, d, J=7.8 Hz), 7.36 (2H, d, J=7.8 Hz), 8.28 (3H, brs).

### Example 96

### N′-[5-(aminomethyl)-6-isobutyl-2-methyl-4-(4-methylphenyl)pyridin-3-yl]-N,N-dimethylurea dihydrochloride

1) tert-Butyl {[5-{[(dimethylamino)carbonyl]amino}-2-isobutyl-6-methyl-4-(4-methylphenyl)pyridin-3-yl]methyl}carbamate (158 mg, yield 35%) was obtained as a white powder from 5-{[(tert-butoxycarbonyl)amino]methyl}-6-isobutyl-2-methyl-4-(4-methylphenyl)nicotinic acid (412 mg, 1.0 mmol) and 2M dimethylamine tetrahydrofuran solution (0.6 mL, 1.2 mmol) according to a method similar to the method of Example 95-1).

¹H-NMR (CDCl₃) δ:0.97 (6H, d, J=6.6 Hz), 1.38 (9H, s), 2.15-2.25 (1H, m), 2.41 (3H, s), 2.51 (3H, s), 2.71 (6H, s), 2.75 (2H, d, J=9.0 Hz), 4.08 (2H, d, J=5.1 Hz), 4.23 (1H, brs), 5.32 (1H, s), 7.02 (2H, d, J=7.8 Hz), 7.24 (2H, d, J=7.8 Hz).

2) N′-[5-(Aminomethyl)-6-isobutyl-2-methyl-4-(4-methylphenyl)pyridin-3-yl]-N,N-dimethylurea dihydrochloride (108 mg, yield 73%) was obtained as a white powder from tert-butyl {[5-{[(dimethylamino)carbonyl]amino}-2-isobutyl-6-methyl-4-(4-methylphenyl)pyridin-3-yl]methyl}carbamate (158 mg, 0.35 mmol) according to a method similar to the method of Example 2-3).

¹H-NMR (DMSO-d₆) δ:0.98 (6H, d, J=6.3 Hz), 2.17-2.20 (1H, m), 2.39 (3H, s), 2.64 (9H, s), 3.09 (2H, brs), 3.83 (2H, brs), 7.20 (2H, d, J=7.8 Hz), 7.31 (2H, d, J=7.8 Hz), 7.86 (1H, brs), 8.39 (3H, brs).

### Example 97

### benzyl [5-(aminomethyl)-6-isobutyl-2-methyl-4-(4-methylphenyl)pyridin-3-yl]carbamate dihydrochloride

1) Benzyl [5-{[(tert-butoxycarbonyl)amino]methyl}-6-isobutyl-2-methyl-4-(4-methylphenyl)pyridin-3-yl]carbamate (1600 mg, yield 35%) was obtained as a white powder from 5-{[(tert-butoxycarbonyl)amino]methyl}-6-isobutyl-2-methyl-4-(4-methylphenyl)nicotinic acid (3700 mg, 8.9 mmol) and benzyl alcohol (2.3 mL, 10.7 mmol) according to a method similar to the method of Example 95-1).

¹H-NMR (CDCl₃) δ:0.97 (6H, d, J=6.6 Hz), 1.38 (9H, s), 2.13-2.16 (1H, m), 2.39 (3H, s), 2.51 (3H, s), 2.75 (2H, d, J=7.2 Hz), 4.08 (2H, s), 4.22 (1H, brs), 5.07 (2H, s), 5.70 (1H, brs), 6.95 (2H, brs), 7.17 (2H, d, J=7.8 Hz), 7.20-7.26 (2H, m), 7.31-7.36 (3H, m).

2) Benzyl [5-(aminomethyl)-6-isobutyl-2-methyl-4-(4-methylphenyl)pyridin-3-yl]carbamate dihydrochloride (54 mg, yield 76%) was obtained as a white powder from benzyl [5-{[(tert-butoxycarbonyl)amino]methyl}-6-isobutyl-2-methyl-4-(4-methylphenyl)pyridin-3-yl]carbamate (75 mg, 0.14 mmol) according to a method similar to the method of Example 2-3).

¹H-NMR (DMSO-d₆) δ:0.97 (6H, d, J=6.3 Hz), 2.15-2.22 (1H, m), 2.39 (3H, s), 2.56 (3H, s), 2.99 (2H, s), 3.79 (2H, s), 5.00 (2H, s), 7.14-7.18 (4H, m), 7.29-7.35 (5H, m), 8.29 (3H, brs), 9.08 (1H, brs).

### Example 98

### 5-(aminomethyl)-6-isobutyl-2-methyl-4-(4-methylphenyl)-3-pyridinamine trihydrochloride

1) To a solution (100 mL) of benzyl [5-{[(tert-butoxycarbonyl)amino]methyl}-6-isobutyl-2-methyl-4-(4-methylphenyl)pyridin-3-yl]carbamate (1500 mg, 2.9 mmol) in ethanol was added 5% palladium-carbon (150 mg) and the mixture was stirred under a hydrogen atmosphere at room temperature for 2 hrs. The reaction mixture was filtered and the filtrate was concentrated under reduced pressure. The obtained residue was purified by silica gel column chromatography to give tert-butyl {[5-amino-2-isobutyl-6-methyl-4-(4-methylphenyl)pyridin-3-yl]methyl}carbamate (1000 mg, yield 90%) as a white powder.

¹H-NMR (CDCl₃) δ:0.94 (6H, d, J=6.6 Hz), 1.38 (9H, s), 2.09-2.16 (1H, m), 2.41 (3H, s), 2.42 (3H, s), 2.65 (2H, d, J=7.2 Hz), 3.28 (2H, s), 4.02 (2H, brs), 4.22 (1H, brs), 7.06 (2H, d, J=8.1 Hz), 7.29 (2H, d, J=7.7 Hz).

2) 5-(Aminomethyl)-6-isobutyl-2-methyl-4-(4-methylphenyl)-3-pyridinamine trihydrochloride (34 mg, yield 62%) was obtained as a white powder from tert-butyl {[5-amino-2-isobutyl-6-methyl-4-(4-methylphenyl)pyridin-3-yl]methyl}carbamate (50 mg, 0.13 mmol) according to a method similar to the method of Example 2-3).

¹H-NMR (DMSO-d₆) δ:0.94 (6H, d, J=6.6 Hz), 1.97-2.08 (1H, m), 2.42 (3H, s), 2.65 (3H, s), 2.99 (2H, s), 3.69 (2H, s), 5.40 (3H, brs), 7.26 (2H, d, J=8.1 Hz), 7.44 (2H, d, J=8.1 Hz), 8.38 (3H, brs).

### Example 99

### N-[5-(aminomethyl)-6-isobutyl-2-methyl-4-(4-methylphenyl)pyridin-3-yl]methanesulfonamide dihydrochloride

To a solution of tert-butyl {[5-amino-2-isobutyl-6-methyl-4-(4-methylphenyl)pyridin-3-yl]methyl}carbamate (100 mg, 0.26 mmol) in tetrahydrofuran (2 mL) was added triethylamine (54 μL, 0.39 mmol) and methanesulfonyl chloride (30 μL, 0.39 mmol) was added at room temperature. Then the mixture was stirred for 3 hrs. Water was added to the reaction mixture, and the mixture was extracted with ethyl acetate. The organic layer was washed with saturated brine and dried over anhydrous magnesium sulfate. The solvent was evaporated under reduced pressure and the obtained residue was purified by silica gel column chromatography to give an oil. To a solution of the oil in ethyl acetate (1 mL) was added 4N hydrogen chloride ethyl acetate solution (1 mL) and the mixture was stirred at room temperature for 1 hr. The solvent was evaporated under reduced pressure and the obtained residue was crystallized from hexane to give N-[5-(aminomethyl)-6-isobutyl-2-methyl-4-(4-methylphenyl)pyridin-3-yl]methanesulfonamide dihydrochloride (25 mg, yield 22%) as a white powder.

¹H-NMR (DMSO-d₆) δ:0.97 (6H, d, J=6.6 Hz), 2.18-2.24 (1H, m), 2.20 (3H, s), 2.39 (3H, s), 2.71 (3H, s), 2.96 (2H, s), 3.79 (2H, s), 7.28 (2H, d, J=6.9 Hz), 7.34 (2H, d, J=6.9 Hz), 8.32 (3H, brs), 9.27 (1H, brs).

### Example 100

### N-[5-({[5-(aminomethyl)-6-isobutyl-2-methyl-4-(4-methylphenyl)pyridin-3-yl]amino}sulfonyl)-4-methyl-1,3-thiazol-2-yl]acetamide dihydrochloride

N-[5-({[5-(Aminomethyl)-6-isobutyl-2-methyl-4-(4-methylphenyl)pyridin-3-yl]amino}sulfonyl)-4-methyl-1,3-thiazol-2-yl]acetamide dihydrochloride (58 mg, yield 39%) was obtained as a white powder from tert-butyl {[5-amino-2-isobutyl-6-methyl-4-(4-methylphenyl)pyridin-3-yl]methyl}carbamate (100 mg, 0.26 mmol) and 2-(acetylamino)-4-methyl-1,3-thiazole-5-sulfonyl chloride (76 mg, 0.3 mmol) according to a method similar to the method of Example 99.

¹H-NMR (DMSO-d₆) δ:0.94 (6H, d, J=6.6 Hz), 2.02 (3H, s), 2.19 (3H, s), 2.18-2.23 (1H, m), 2.27 (3H, s), 2.53 (3H, s), 2.84 (2H, brs), 3.69 (2H, brs), 6.92-6.97 (4H, m), 8.10 (3H, brs), 9.89 (1H, brs).

### Example 101

### {[5-(aminomethyl)-2-isobutyl-6-methyl-4-(4-methylphenyl)pyridin-3-yl]methyl}amine trihydrochloride

1) A mixture of tert-butyl {[5-(hydroxymethyl)-2-isobutyl-6-methyl-4-(4-methylphenyl)pyridin-3-yl]methyl}carbamate (1.16 g, 2.91 mmol), triethylamine (0.8 mL, 5.82 mmol) and tetrahydrofuran (15 mL) was cooled to 0° C. and methanesulfonyl chloride (500 mg, 4.37 mmol) was added dropwise. After stirring at room temperature for 30 min., the reaction mixture was poured into saturated aqueous sodium hydrogen carbonate, and the mixture was extracted with ethyl acetate. The extract was dried over anhydrous magnesium sulfate and the solvent was evaporated under reduced pressure to give [5-{[(tert-butoxycarbonyl)amino]methyl}-6-isobutyl-2-methyl-4-(4-methylphenyl)pyridin-3-yl]methyl methanesulfonate as a crude product. The crude product was dissolved in N,N-dimethylformamide (30 mL) and sodium azide (379 mg, 5.82 mmol) was added. The mixture was stirred at 80° C. for 30 min. The reaction mixture was diluted with ethyl acetate (100 mL) and washed with saturated brine. The organic layer was dried over anhydrous magnesium sulfate and the solvent was evaporated under reduced pressure to give a residue. A mixture of the obtained residue, 10% palladium-carbon (304 mg, 0.291 mmol) and ethanol (15 mL) was stirred under a hydrogen atmosphere at room temperature for 2 hrs. After filtration, the solvent was evaporated under reduced pressure and the obtained residue was purified by silica gel column chromatography to give tert-butyl {[5-(aminomethyl)-2-isobutyl-6-methyl-4-(4-methylphenyl)pyridin-3-yl]methyl}carbamate (690 mg, yield 60%) as a yellow oil.

¹H-NMR (CDCl₃) δ:0.96 (6H, d, J=6.6 Hz), 1.38 (9H, s), 1.41 (2H, brs), 2.14-2.23 (1H, m), 2.41 (3H, s), 2.64 (3H, s), 4.02 (2H, d, J=5.1 Hz), 4.18 (1H, brs), 7.02 (2H, d, J=7.9 Hz), 7.25 (2H, d, J=7.0 Hz).

2) {[5-(Aminomethyl)-2-isobutyl-6-methyl-4-(4-methylphenyl)pyridin-3-yl]methyl}amine trihydrochloride (204 mg, yield 99%) was obtained as a white powder from tert-butyl {[5-(aminomethyl)-2-isobutyl-6-methyl-4-(4-methylphenyl)pyridin-3-yl]methyl}carbamate (200 mg, 0.503 mmol) according to a method similar to the method of Example 2-3).

¹H-NMR (DMSO-d₆) δ:0.97 (6H, d, J=6.6 Hz), 2.13-2.24 (1H, m), 2.43 (3H, s), 2.50 (3H, s), 2.98 (2H, brs), 3.76 (4H, brs), 7.34-7.45 (4H, m), 8.51 (6H, brs).

### Example 102

### N-{[5-(aminomethyl)-6-isobutyl-2-methyl-4-(4-methylphenyl)pyridin-3-yl]methyl}-4-(methylsulfonyl)benzenesulfonamide dihydrochloride

1) To a solution (10 mL) of tert-butyl {[5-(aminomethyl)-2-isobutyl-6-methyl-4-(4-methylphenyl)pyridin-3-yl]methyl}carbamate (290 mg, 0.729 mmol) and triethylamine (0.15 mL, 1.09 mmol) in tetrahydrofuran was added 4-(methylsulfonyl)benzenesulfonyl chloride (223 mg, 0.875 mmol) and the mixture was stirred at room temperature for 1 hr. The reaction mixture was diluted with ethyl acetate (100 mL) and washed successively with saturated aqueous sodium hydrogen carbonate and saturated brine. The organic layer was dried over anhydrous magnesium sulfate. The solvent was evaporated under reduced pressure and the obtained yellow solid was washed with diisopropyl ether to give tert-butyl ({2-isobutyl-6-methyl-4-(4-methylphenyl)-5-[({[4-(methylsulfonyl)phenyl]sulfonyl}amino)methyl]pyridin-3-yl}methyl)carbamate (391 mg, yield 87%) as a yellow powder.

¹H-NMR (CDCl₃) δ:0.95 (6H, d, J=6.6 Hz), 1.36 (9H, s), 2.13-2.22 (1H, m), 2.41 (3H, s), 2.61 (3H, s), 2.73 (2H, d, J=7.4 Hz), 3.08 (3H, s), 3.83 (2H, d, J=5.8 Hz), 3.97 (2H, d, J=4.9 Hz), 4.11-4.20 (2H, m), 6.84 (2H, d, J=8.1 Hz), 7.13 (2H, d, J=7.7 Hz), 7.77 (2H, d, J=8.7 Hz), 7.98 (2H, d, J=8.5 Hz).

2) N-{[5-(Aminomethyl)-6-isobutyl-2-methyl-4-(4-methylphenyl)pyridin-3-yl]methyl}-4-(methylsulfonyl)benzenesulfonamide dihydrochloride (370 mg, yield 99%) was obtained as a yellow powder from tert-butyl ({2-isobutyl-6-methyl-4-(4-methylphenyl)-5-[({[4-(methylsulfonyl)phenyl]sulfonyl}amino)methyl]pyridin-3-yl}methyl)carbamate (391 mg, 0.635 mmol) according to a method similar to the method of Example 2-3).

¹H-NMR (DMSO-d₆) δ:0.96 (6H, d, J=6.6 Hz), 2.11-2.19 (1H, m), 2.35 (3H, s), 2.50 (3H, s), 2.70-2.82 (2H, m), 3.31 (3H, s), 3.66 (2H, brs), 3.72 (2H, brs), 7.11-7.21 (4H, m), 7.83 (2H, dd, J=8.3, 1.3 Hz), 8.08 (2H, d, J=8.1 Hz), 8.31 (3H, brs).

### Example 103

### ethyl ({[5-(aminomethyl)-6-isobutyl-2-methyl-4-(4-methylphenyl)pyridin-3-yl]methyl}amino)acetate trihydrochloride

1) To a solution of [5-{[(tert-butoxycarbonyl)amino]methyl}-6-isobutyl-2-methyl-4-(4-methylphenyl)pyridin-3-yl]methyl methanesulfonate (300 mg, 0.63 mmol) in tetrahydrofuran (5 mL) were added triethylamine (223 μL, 1.6 mmol) and glycine ethyl ester hydrochloride (100 mg, 0.7 mmol) and the mixture was stirred at 60° C. for 3 days. Water was added to the reaction mixture, and the mixture was extracted with ethyl acetate. The organic layer was washed with saturated brine and dried over anhydrous magnesium sulfate. The solvent was evaporated under reduced pressure and the obtained residue was purified by silica gel column chromatography to give ethyl ({[5-{[(tert-butoxycarbonyl)amino]methyl}-6-isobutyl-2-methyl-4-(4-methylphenyl)pyridin-3-yl]methyl}amino)acetate (185 mg, yield 61%) as a white powder.

¹H-NMR (CDCl₃) δ:0.95 (6H, d, J=6.6 Hz), 1.22 (3H, t, J=6.9 Hz), 1.38 (9H, s), 2.15-2.22 (1H, m), 2.41 (3H, s), 2.67 (3H, s), 2.73 (2H, d, J=7.2 Hz), 3.18 (2H, s), 3.43 (2H, s), 4.02 (2H, s), 4.09 (2H, q, J=6.9 Hz), 4.18 (1H, brs), 7.03 (2H, d, J=7.8 Hz), 7.25 (2H, d, J=7.8 Hz).

2) Ethyl ({[5-(aminomethyl)-6-isobutyl-2-methyl-4-(4-methylphenyl)pyridin-3-yl]methyl}amino)acetate trihydrochloride (57 mg, yield 95%) was obtained as a white powder from ethyl ({[5-{[(tert-butoxycarbonyl)amino]methyl}-6-isobutyl-2-methyl-4-(4-methylphenyl)pyridin-3-yl]methyl}amino)acetate (60 mg, 0.12 mmol) according to a method similar to the method of Example 2-3).

¹H-NMR (DMSO-d₆) δ:0.97 (6H, d, J=6.6 Hz), 1.18 (3H, t, J=6.9 Hz), 2.11-2.24 (1H, m), 2.42 (3H, s), 2.92 (3H, brs), 3.03 (2H, brs), 3.61 (2H, s), 3.72 (2H, brs), 4.06 (2H, s), 4.08 (2H, q, J=6.9 Hz), 7.35 (2H, d, J=8.1 Hz), 7.40 (2H, d, J=8.1 Hz), 8.43 (3H, brs).

### Example 104

### ({[5-(aminomethyl)-6-isobutyl-2-methyl-4-(4-methylphenyl)pyridin-3-yl]methyl}amino)acetic acid trihydrochloride

1) To a solution of ethyl ({[5-([(tert-butoxycarbonyl)amino]methyl}-6-isobutyl-2-methyl-4-(4-methylphenyl)pyridin-3-yl]methyl}amino)acetate (100 mg, 0.2 mmol) in ethanol (3 mL) was added 8N aqueous sodium hydroxide solution (3 mL) and the mixture was stirred at 80° C. for 15 hrs. 1N Hydrochloric acid was added to neutralize the reaction mixture and the mixture was extracted with ethyl acetate. The organic layer was washed with saturated brine and dried over anhydrous magnesium sulfate. The solvent was evaporated under reduced pressure to give ({[5-{[(tert-butoxycarbonyl)amino]methyl}-6-isobutyl-2-methyl-4-(4-methylphenyl)pyridin-3-yl]methyl}amino)acetic acid (92 mg, yield 99%) as a white powder.

¹H-NMR (DMSO-d₆) δ:0.91 (6H, d, J=6.3 Hz), 1.35 (9H, s), 2.11-2.24 (1H, m), 2.36 (3H, s), 2.54 (2H, s), 2.57 (3H, s), 2.97 (2H, s), 3.39 (2H, s), 3.76 (2H, s), 6.78 (1H, brs), 7.18 (2H, d, J=7.8 Hz), 7.22 (2H, d, J=7.8 Hz).

2) ({[5-(Aminomethyl)-6-isobutyl-2-methyl-4-(4-methylphenyl)pyridin-3-yl]methyl}amino)acetic acid trihydrochloride (75 mg, yield 80%) was obtained as a white powder from ({[5-{[(tert-butoxycarbonyl)amino]methyl}-6-isobutyl-2-methyl-4-(4-methylphenyl)pyridin-3-yl]methyl}amino)acetic acid (90 mg, 0.2 mmol) according to a method similar to the method of Example 2-3).

¹H-NMR (DMSO-d₆) δ:0.97 (6H, d, J=6.6 Hz), 2.14-2.21 (1H, m), 2.42 (3H, s), 2.89 (3H, s), 3.01 (2H, brs), 3.52 (2H, s), 3.72 (2H, s), 4.04 (2H, s), 7.35 (2H, d, J=8.1 Hz), 7.39 (2H, d, J=8.1 Hz), 8.37 (3H, brs), 9.29 (1H, brs).

### Example 105

### 4-{[5-(aminomethyl)-6-isobutyl-2-methyl-4-(4-methylphenyl)pyridin-3-yl]methyl}-2-piperazinone trihydrochloride

1) tert-Butyl ({2-isobutyl-6-methyl-4-(4-methylphenyl)-5-[(3-oxo-1-piperazinyl)methyl]pyridin-3-yl}methyl)carbamate (78 mg, yield 77%) was obtained as a white powder from [5-{[(tert-butoxycarbonyl)amino]methyl}-6-isobutyl-2-methyl-4-(4-methylphenyl)pyridin-3-yl]methyl methanesulfonate (300 mg, 0.63 mmol) and 2-piperazinone (65 mg, 0.65 mmol) according to a method similar to the method of Example 103-1).

¹H-NMR (CDCl₃) δ:0.96 (6H, d, J=6.6 Hz), 1.39 (9H, s), 2.14-2.23 (1H, m), 2.49 (5H, s), 2.64 (3H, s), 2.73 (2H, d, J=7.2 Hz), 2.89 (2H, s), 3.22 (2H, brs), 3.28 (2H, s), 4.01 (2H, d, J=5.1 Hz), 4.20 (1H, brs), 5.69 (1H, brs), 6.96 (2H, d, J=7.8 Hz), 7.21 (2H, d, J=7.8 Hz).

2) 4-{[5-(Aminomethyl)-6-isobutyl-2-methyl-4-(4-methylphenyl)pyridin-3-yl]methyl}-2-piperazinone trihydrochloride (64 mg, yield 87%) was obtained as a white powder from tert-butyl ({2-isobutyl-6-methyl-4-(4-methylphenyl)-5-[(3-oxo-1-piperazinyl)methyl]pyridin-3-yl}methyl)carbamate (75 mg, 0.15 mmol) according to a method similar to the method of Example 2-3).

¹H-NMR (DMSO-d₆) δ:0.98 (6H, d, J=6.6 Hz), 1.91 (2H, s), 2.09-2.14 (1H, m), 2.42 (3H, s), 3.00 (3H, brs), 3.18 (4H, brs), 3.75 (2H, brs), 7.30 (2H, d, J=7.5 Hz), 7.41 (2H, d, J=7.5 Hz), 7.41 (1H, brs), 8.52 (3H, brs).

### Example 106

### 3-{[5-(aminomethyl)-6-isobutyl-2-methyl-4-(4-methylphenyl)pyridin-3-yl]methyl}-2,4-imidazolidinedione dihydrochloride

1) To a solution of tert-butyl {[5-(hydroxymethyl)-2-isobutyl-6-methyl-4-(4-methylphenyl)pyridin-3-yl]methyl}carbamate (100 mg, 0.25 mmol), hydantoin (38 mg, 0.38 mmol) and tributylphosphine (95 μL, 0.38 mmol) in tetrahydrofuran (3 mL) was added 1,1′-(azodicarbonyl)dipiperidine (96 mg, 0.38 mmol) and the mixture was stirred at room temperature for 4 hrs. The reaction mixture was concentrated and insoluble materials were filtered off. The filtrate was purified by silica gel column chromatography to give tert-butyl {[5-[(2,5-dioxo-1-imidazolidinyl)methyl]-2-isobutyl-6-methyl-4-(4-methylphenyl)pyridin-3-yl]methyl}carbamate (68 mg, yield 57%) as a white powder.

¹H-NMR (CDCl₃) δ:0.95 (6H, d, J=6.6 Hz), 1.38 (9H, s), 2.11-2.26 (1H, m), 2.39 (3H, s), 2.55 (3H, s), 2.73 (2H, d, J=7.5 Hz), 3.77 (2H, s), 3.99 (2H, d, J=5.1 Hz), 4.23 (1H, brs), 4.46 (2H, s), 5.10 (1H, brs), 7.07 (2H, d, J=7.8 Hz), 7.23 (2H, d, J=7.8 Hz).

2) 3-{[5-(Aminomethyl)-6-isobutyl-2-methyl-4-(4-methylphenyl)pyridin-3-yl]methyl}-2,4-imidazolidinedione dihydrochloride (54 mg, yield 95%) was obtained as a white powder from tert-butyl {[5-[(2,5-dioxo-1-imidazolidinyl)methyl]-2-isobutyl-6-methyl-4-(4-methylphenyl)pyridin-3-yl]methyl}carbamate according to a method similar to the method of Example 2-3).

¹H-NMR (DMSO-d₆) δ:0.96 (6H, d, J=6.6 Hz), 2.14-2.19 (1H, m), 2.37 (3H, s), 2.84 (3H, s), 3.11 (2H, brs), 3.71 (4H, s), 4.35 (2H, s), 7.18 (2H, d, J=8.1 Hz), 7.33 (2H, d, J=7.8 Hz), 8.00 (1H, brs), 8.30 (1H, brs).

### Example 107

### 1-{[5-(aminomethyl)-6-isobutyl-2-methyl-4-(4-methylphenyl)pyridin-3-yl]methyl}-2,5-piperazinedione dihydrochloride

1) To a solution of Z-glycine (1.2 g, 6 mmol) and N,N-dimethylformamide (10 μL) in tetrahydrofuran (5 mL) was added oxalyl chloride (530 μL, 6 mmol), and the mixture was stirred at room temperature for 30 min. The reaction mixture was added dropwise to a solution of ethyl ({[5-{[(tert-butoxycarbonyl)amino]methyl}-6-isobutyl-2-methyl-4-(4-methylphenyl)pyridin-3-yl]methyl}amino)acetate (1.4 g, 3 mmol), pyridine (970 μL, 12 mmol) and 4-dimethylaminopyridine (5 mg) in tetrahydrofuran (10 mL) under ice-cooling and the mixture was stirred for 3 hrs. Water was added to the reaction mixture and the mixture was extracted with ethyl acetate. The organic layer was washed with saturated brine and dried over anhydrous magnesium sulfate. The solvent was evaporated under reduced pressure and the obtained oil was dissolved in ethanol (10 mL). 5% Palladium-carbon (100 mg) was added and the mixture was stirred under a hydrogen atmosphere at room temperature for 2 hrs. The reaction mixture was filtered and the filtrate was concentrated under reduced pressure. The obtained residue was purified by silica gel column chromatography to give tert-butyl {[5-[(2,5-dioxo-1-piperazinyl)methyl]-2-isobutyl-6-methyl-4-(4-methylphenyl)pyridin-3-yl]methyl}carbamate (35 mg, yield 2.4%) as a white powder.

¹H-NMR (CDCl₃) δ:0.97 (6H, d, J=6.6 Hz), 1.39 (9H, s), 2.18-2.24 (1H, m), 2.40 (3H, s), 2.51 (3H, s), 2.76 (2H, d, J=7.5 Hz), 3.47 (2H, s), 3.93 (2H, s), 4.03 (2H, d, J=5.1 Hz), 4.24 (1H, brs), 4.51 (2H, s), 5.88 (1H, brs), 6.98 (2H, d, J=7.5 Hz), 7.25 (2H, d, J=7.5 Hz).

2) 1-{[5-(Aminomethyl)-6-isobutyl-2-methyl-4-(4-methylphenyl)pyridin-3-yl]methyl}-2,5-piperazinedione dihydrochloride (14 mg, yield 60%) was obtained as a white powder from tert-butyl {[5-[(2,5-dioxo-1-piperazinyl)methyl]-2-isobutyl-6-methyl-4-(4-methylphenyl)pyridin-3-yl]methyl}carbamate according to a method similar to the method of Example 2-3).

¹H-NMR (DMSO-d₆) δ:0.97 (6H, d, J=6.6 Hz), 2.15-2.19 (1H, m), 2.39 (3H, s), 2.69 (3H, s), 3.25 (2H, s), 3.67 (2H, s), 3.73 (2H, brs), 4.31 (2H, s), 7.18 (2H, d, J=8.1 Hz), 7.37 (2H, d, J=7.8 Hz), 8.06 (1H, brs), 8.24 (3H, brs).

### Example 108

### {[2-isobutyl-4-(4-methylphenyl)-6-phenylpyridin-3-yl]methyl}amine dihydrochloride

1) To a solution (140 mL) of acetophenone (8.40 g, 70 mmol) and p-tolualdehyde (8.40 g, 70 mmol) in ethanol was added sodium hydroxide (7.0 g, 175 mmol) and the mixture was stirred for 3 days. The reaction mixture was diluted with ethyl acetate (100 mL) and washed with saturated brine. The organic layer was dried over anhydrous magnesium sulfate. The solvent was evaporated under reduced pressure and the obtained yellow solid was washed with diisopropyl ether to give (2E)-3-(4-methylphenyl)-1-phenylprop-2-en-1-one (9.12 g, yield 59%) as a yellow powder.

¹H-NMR (CDCl₃) δ:2.40 (3H, s), 7.23 (2H, d, J=8.1 Hz), 7.47-7.62 (6H, m), 7.80 (1H, d, J=15.8 Hz), 8.00-8.03 (2H, m).

2) A mixture of 5-methyl-3-oxohexanenitrile (5.0 g, 40 mmol), acetic acid (2.3 mL, 40 mmol), ammonium acetate (15.4 g, 200 mmol) and toluene (250 mL) was heated under reflux using a Dean-Stark trap for 12 hrs. The reaction mixture was allowed to cool to room temperature, washed with saturated brine and dried over anhydrous magnesium sulfate. The solvent was evaporated under reduced pressure to give a residue (4.5 g). The residue (2.25 g) was dissolved in ethanol (100 mL) and (2E)-3-(4-methylphenyl)-1-phenylprop-2-en-1-one (3.69 g, 16.6 mmol) and sodium hydroxide (0.8 g, 20 mmol) were added. The mixture was heated under reflux for 3 hrs. The reaction mixture was diluted with ethyl acetate (100 mL) and washed with saturated aqueous ammonium chloride. The organic layer was dried over anhydrous magnesium sulfate and the solvent was evaporated under reduced pressure. The obtained residue was purified by silica gel column chromatography to give 2-isobutyl-4-(4-methylphenyl)-6-phenylnicotinonitrile (2.68 g, yield 49%) as a yellow oil.

¹H-NMR (CDCl₃) δ:1.07 (6H, d, J=6.8 Hz), 2.35-2.48 (4H, m), 3.06 (2H, d, J=7.2 Hz), 7.35 (2H, d, J=7.9 Hz), 7.49-7.56 (5H, m), 7.67 (1H, s), 8.07-8.13 (1H, m).

3) {[2-Isobutyl-4-(4-methylphenyl)-6-phenylpyridin-3-yl]methyl}amine (1.70 g, yield 63%) was obtained as a yellow oil from 2-isobutyl-4-(4-methylphenyl)-6-phenylnicotinonitrile (2.65 g, 8.12 mmol) according to a method similar to the method of Example 1-4). The oil was dissolved in 4N hydrogen chloride 1,4-dioxane solution (20 mL) and the solvent was evaporated under reduced pressure. The obtained yellow solid was washed with diisopropyl ether to give {[2-isobutyl-4-(4-methylphenyl)-6-phenylpyridin-3-yl]methyl}amine dihydrochloride (1.99 g, yield 96%) as a yellow powder.

¹H-NMR (DMSO-d₆) δ:1.03 (6H, d, J=6.6 Hz), 2.34-2.41 (4H, m), 2.94 (2H, d, J=7.0 Hz), 4.00 (2H, d, J=5.5 Hz), 7.36 (2H, d, J=8.2 Hz), 7.41 (2H, d, J=8.3 Hz), 7.47-7.54 (3H, m), 7.70 (1H, s), 8.15 (2H, dd, J=7.9, 1.5 Hz), 8.43 (3H, brs).

### Example 109

### 5-(aminomethyl)-6-isobutyl-2-methyl-4-(4-methylphenyl)nicotinic acid maleate

5-(Aminomethyl)-6-isobutyl-2-methyl-4-(4-methylphenyl)nicotinic acid (1.50 g, 4.80 mmol) was dissolved in a mixed solvent of water (15 mL) and acetonitrile (15 mL) and the mixture was heated under reflux for 10 min. Maleic acid (558 mg, 4.80 mmol) was added to the obtained solution and the mixture was stirred at the same temperature for 10 min. Acetonitrile (200 mL) was added to the obtained solution, and the mixture was allowed to cool to room temperature and stirred at 0° C. for 30 min. The precipitated solid was collected by filtration and washed with acetonitrile (30 mL) to give 5-(aminomethyl)-6-isobutyl-2-methyl-4-(4-methylphenyl)nicotinic acid maleate (667 mg, yield 32%) as a white powder.

¹H-NMR (DMSO-d₆) δ:0.96 (6H, d, J=6.6 Hz), 2.18-2.27 (1H, m), 2.37 (3H, s), 2.74 (2H, d, J=7.0 Hz), 3.79 (2H, s), 6.01 (2H, s), 7.19 (2H, d, J=7.9 Hz), 7.29 (2H, d, J=7.5 Hz).

### Example 110

### 5-(aminomethyl)-6-(methoxymethyl)-2-methyl-4-(4-methylphenyl)nicotinic acid dihydrochloride

1) A solution (40 mL) of methyl 4-methoxyacetoacetate (5.85 g, 40 mmol), p-tolualdehyde (4.81 g, 40 mmol), piperidine (340 mg, 4 mmol) and acetic acid (240 mg, 4 mmol) in isopropanol was stirred at room temperature for 3 days. The solvent was evaporated under reduced pressure to give a residue. 3-Methyl 5-tert-butyl 2-(methoxymethyl)-6-methyl-4-(4-methylphenyl)-1,4-dihydropyridine-3,5-dicarboxylate (5.85 g, yield 50%) was obtained as a yellow oil from the obtained residue and tert-butyl 3-aminocrotonate (4.71 g, 30.0 mol) according to a method similar to the method of Example 1-2). That is, the aforementioned residue and tert-butyl 3-aminocrotonate were dissolved in methanol (30 mL) and the mixture was heated under reflux for 1.5 hrs. The reaction mixture was concentrated under reduced pressure and the residue was purified by silica gel column chromatography to give 3-methyl 5-tert-butyl 2-(methoxymethyl)-6-methyl-4-(4-methylphenyl)-1,4-dihydropyridine-3,5-dicarboxylate.

¹H-NMR (CDCl₃) δ:1.40 (9H, s), 2.28 (3H, s), 2.32 (3H, s), 3.45-3.46 (3H, m), 3.62-3.63 (3H, m), 4.55-4.76 (2H, m), 4.89-4.95 (1H, m), 6.94 (1H, brs), 7.01 (2H, d, J=7.7 Hz), 7.15 (2H, d, J=8.1 Hz).

2) 3-Methyl 5-tert-butyl 2-(methoxymethyl)-6-methyl-4-(4-methylphenyl)pyridine-3,5-dicarboxylate (3.78 g, yield 65%) was obtained as a yellow oil from 3-methyl 5-tert-butyl 2-(methoxymethyl)-6-methyl-4-(4-methylphenyl)-1,4-dihydropyridine-3,5-dicarboxylate (5.85 g, 15.1 mmol) according to a method similar to the method of Example 23-3).

¹H-NMR (CDCl₃) δ:1.23 (9H, s), 2.37 (3H, s), 2.61 (3H, s), 3.36 (3H, s), 3.54 (3H, s), 4.66 (2H, s), 7.13-7.15 (2H, m), 7.17-7.19 (2H, m).

3) A suspension of 3-methyl 5-tert-butyl 2-(methoxymethyl)-6-methyl-4-(4-methylphenyl)pyridine-3,5-dicarboxylate (3.78 g, 9.81 mmol) in toluene (50 mL) was cooled to −78° C. and 1.50 M diisobutylaluminum hydride toluene solution (25 mL, 24.5 mmol) was added dropwise over 15 min. The mixture was stirred at −78° C. for 30 min., allowed to warm to 0° C. and further stirred for 10 min. Methanol (0.5 mL) was added to the reaction mixture and sodium sulfate 10 hydrate (8.1 g, 9.8 mmol) was added. The mixture was stirred at room temperature for 1 hr. The insoluble material was filtered off and the filtrate was concentrated under reduced pressure. The residue was purified by silica gel column chromatography to give tert-butyl 5-(hydroxymethyl)-6-(methoxymethyl)-2-methyl-4-(4-methylphenyl)nicotinate (810 mg, yield 23%) as a yellow oil.

¹H-NMR (CDCl₃) δ:1.21 (9H, s), 2.39 (3H, s), 2.59 (3H, s), 3.50 (3H, s), 4.39 (2H, d, J=6.8 Hz), 4.76 (2H, s), 7.21 (4H, s).

4) A mixture of tert-butyl 5-(hydroxymethyl)-6-(methoxymethyl)-2-methyl-4-(4-methylphenyl)nicotinate (810 mg, 2.27 rnmol), triethylamine (0.63 mL, 4.54 mmol) and tetrahydrofuran (30 mL) was cooled to 0° C. and methanesulfonyl chloride (0.26 mL, 3.40 mmol) was added dropwise. After stirring at room temperature for 30 min., the reaction mixture was diluted with ethyl acetate (100 mL) and washed with saturated aqueous sodium hydrogen carbonate. The organic layer was dried over anhydrous magnesium sulfate, and the solvent was evaporated under reduced pressure. The residue was dissolved in N,N-dimethylformamide (20 mL) and sodium azide (296 mg, 4.54 mmol) was added. The mixture was stirred at 80° C. for 1 hr. Ethyl acetate was added to the reaction mixture, and the mixture was washed successively with water and saturated brine and dried over anhydrous magnesium sulfate. The solvent was evaporated under reduced pressure. A mixture of the residue, 10% palladium-carbon (242 mg, 0.227 mmol) and ethanol (30 mL) was stirred under a hydrogen atmosphere at room temperature for 30 min. After filtration, the solvent was evaporated under reduced pressure and the obtained residue was purified by silica gel column chromatography to give tert-butyl 5-(aminomethyl)-6-(methoxymethyl)-2-methyl-4-(4-methylphenyl)nicotinate (600 mg, yield 74%) as a yellow oil.

¹H-NMR (CDCl₃) δ:1.19 (9H, s), 2.40 (3H, s), 2.57 (3H, s), 3.48 (3H, s), 3.63 (2H, s), 4.69 (2H, s), 7.12 (2H, d, J=8.1 Hz), 7.23 (2H, d, J=7.7 Hz).

5) 5-(Aminomethyl)-6-(methoxymethyl)-2-methyl-4-(4-methylphenyl)nicotinic acid dihydrochloride (533 mg, yield 84%) was obtained as a white powder from tert-butyl 5-(aminomethyl)-6-(methoxymethyl)-2-methyl-4-(4-methylphenyl)nicotinate (600 mg, 1.69 mmol) according to a method similar to the method of Example 24-1).

¹H-NMR (DMSO-d₆) δ:2.37 (3H, s), 2.53 (3H, s), 3.41 (3H, s), 3.86 (2H, d, J=5.7 Hz), 4.76 (2H, s), 7.24 (2H, d, J=8.1 Hz), 7.30 (2H, d, J=8.1 Hz), 8.10 (3H, brs).

### Example 111

### 5,6-bis(aminomethyl)-2-methyl-4-(4-methylphenyl)nicotinic acid trihydrochloride

1) Ethyl 3-amino-4-[(tert-butoxycarbonyl)amino]but-2-enoate (5.37g, yield 99%) was obtained as a yellow oil from ethyl 4-[(tert-butoxycarbonyl)amino]-3-oxobutanoate (5.4 g, 22.0 mmol) according to a method similar to the method of Example 108-2).

¹H-NMR (CDCl₃) δ:1.26 (3H, t, J=7.2 Hz), 1.46 (9H, s), 3.77 (2H, d, J=6.6 Hz), 4.12 (2H, q, J=7.1 Hz), 4.55 (1H, s).

2) A mixture of tert-butyl acetoacetate (4.75 g, 30 mmol), p-tolualdehyde (4.51 g, 37.5 mmol), piperidine (0.30 mL, 3.00 mmol) and ethanol (0.2 mL) was stirred at room temperature for one day. The reaction mixture was diluted with ethyl acetate (100 mL) and washed with saturated brine. The organic layer was dried over anhydrous magnesium sulfate and the solvent was evaporated under reduced pressure. The obtained residue and ethyl 3-amino-4-[(tert-butoxycarbonyl)amino]but-2-enoate (5.37 g, 22.0 mmol) were stirred at 80° C. for 30 min. and further stirred at 130° C. for 3 hrs. The obtained mixture was purified by silica gel column chromatography to give 3-ethyl 5-tert-butyl 2-{[(tert-butoxycarbonyl)amino]methyl}-6-methyl-4-(4-methylphenyl)-1,4-dihydropyridine-3,5-dicarboxylate (1.95 g, yield 18%) as a yellow oil.

¹H-NMR (CDCl₃) δ:1.22-1.28 (3H, m), 1.40 (9H, s), 1.46 (9H, s), 2.27 (6H, s), 4.04-4.18 (3H, m), 4.37-4.44 (1H, m), 4.87 (1H, s), 5.35 (1H, brs), 7.01 (2H, d, J=7.9 Hz), 7.15 (2H, d, J=8.1 Hz).

3) 3-Ethyl 5-tert-butyl 2-{[(tert-butoxycarbonyl)amino]methyl}-6-methyl-4-(4-methylphenyl)pyridine-3,5-dicarboxylate (1.94 g, yield 99%) was obtained as a yellow oil from 3-ethyl 5-tert-butyl 2-{[(tert-butoxycarbonyl)amino]methyl}-6-methyl-4-(4-methylphenyl)-1,4-dihydropyridine-3,5-dicarboxylate (1.95 g, 4.01 mmol) according to a method similar to the method of Example 23-3).

¹H-NMR (CDCl₃) δ:0.93 (3H, t, J=7.2 Hz), 1.23 (9H, s), 1.47 (9H, s), 2.37 (3H, s), 2.61 (3H, s), 4.02 (2H, q, J=7.1 Hz), 4.50 (2H, d, J=4.7 Hz), 5.87 (1H, brs), 7.13 (2H, d, J=8.3 Hz), 7.17 (2H, d, J=8.3 Hz).

4) tert-Butyl 6-{[(tert-butoxycarbonyl)amino]methyl}-5-(hydroxymethyl)-2-methyl-4-(4-methylphenyl)nicotinate (1.45 g, yield 82%) was obtained as a yellow oil from 3-ethyl 5-tert-butyl 2-{[(tert-butoxycarbonyl)amino]methyl}-6-methyl-4-(4-methylphenyl)pyridine-3,5-dicarboxylate (1.94 g, 4.00 mmol) according to a method similar to the method of Example 110-3).

¹H-NMR (CDCl₃) δ:1.20 (9H, s), 1.46 (9H, s), 2.39 (3H, s), 2.57 (3H, s), 3.38 (1H, brs), 4.46 (2H, d, J=6.0 Hz), 4.54 (2H, d, J=5.8 Hz), 5.87 (1H, brs), 7.18 (2H, d, J=8.3 Hz), 7.21 (2H, d, J=8.3 Hz).

5) tert-Butyl 5-(aminomethyl)-6-{[(tert-butoxycarbonyl)amino]methyl}-2-methyl-4-(4-methylphenyl)nicotinate (580 mg, yield 40%) was obtained as a white powder from tert-butyl 6-{[(tert-butoxycarbonyl)amino]methyl}-5-(hydroxymethyl)-2-methyl-4-(4-methylphenyl)nicotinate (1.45 g, 3.28 mmol) according to a method similar to the method of Example 110-4).

¹H-NMR (CDCl₃) δ:1.18 (9H, s), 1.49 (9H, s), 2.39 (3H, s), 2.56 (3H, s), 3.62 (2H, s), 4.58 (2H, d, J=4.7 Hz), 6.22 (1H, brs), 7.10 (2H, d, J=8.1 Hz), 7.22 (2H, d, J=7.9 Hz).

6) 5,6-Bis(aminomethyl)-2-methyl-4-(4-methylphenyl)nicotinic acid trihydrochloride (510 mg, yield 99%) was obtained as a yellow solid from tert-butyl 5-(aminomethyl)-6-{[(tert-butoxycarbonyl)amino]methyl}-2-methyl-4-(4-methylphenyl)nicotinate (580 mg, 1.31 mmol) according to a method similar to the method of Example 24-1).

¹H-NMR (DMSO-d₆) δ:2.37 (3H, s), 2.57 (3H, s), 3.84-3.89 (2H, m), 4.51-4.61 (2H, m), 7.23 (2H, d, J=7.9 Hz), 7.31 (2H, d, J=7.9 Hz), 8.42 (3H, brs), 8.54 (3H, brs).

### Example 112

### 5-(aminomethyl)-6-hydroxy-2-methyl-4-(4-methylphenyl)nicotinic acid hydrochloride

1) A mixture of tert-butyl acetoacetate (4.75 g, 30 mmol), p-tolualdehyde (4.51 g, 37.5 mmol), piperidine (0.30 mL, 3.00 mmol) and ethanol (0.2 mL) was stirred at room temperature for one day. The reaction mixture was diluted with ethyl acetate (100 mL) and washed with saturated brine. The organic layer was dried over anhydrous magnesium sulfate and the solvent was evaporated under reduced pressure. The obtained residue, ethyl cyanoacetate (6.79 g, 60.0 mmol) and ammonium acetate (11.6 g, 150 mmol) were stirred at 140° C. for 3 hrs. The reaction mixture was diluted with ethyl acetate (100 mL) and washed with saturated aqueous sodium hydrogen carbonate. The organic layer was dried over anhydrous magnesium sulfate and the solvent was evaporated under reduced pressure. The obtained residue was purified by silica gel column chromatography to give tert-butyl 5-cyano-6-hydroxy-2-methyl-4-(4-methylphenyl)nicotinate (0.87 g, yield 9%) as a white solid.

¹H-NMR (CDCl₃) δ:1.19 (9H, s), 2.41 (3H, s), 2.57 (3H, s), 7.24-7.31 (4H, m).

2) tert-Butyl 5-(aminomethyl)-6-hydroxy-2-methyl-4-(4-methylphenyl)nicotinate was obtained as a white solid from tert-butyl 5-cyano-6-hydroxy-2-methyl-4-(4-methylphenyl)nicotinate (0.50 g, 1.54 mmol) according to a method similar to the method of Example 1-4). Subsequently, tert-butyl 5-{[(tert-butoxycarbonyl)amino]methyl}-6-hydroxy-2-methyl-4-(4-methylphenyl)nicotinate (210 mg, yield 32%) was obtained as a colorless oil according to a method similar to the method of Example 2-1).

¹H-NMR (CDCl₃) δ:1.13 (9H, s), 1.39 (9H, s), 2.38 (3H, s), 2.43 (3H, s), 4.02 (2H, d, J=5.8 Hz), 7.10 (2H, d, J=7.9 Hz), 7.22 (2H, d, J=7.9 Hz), 12.39 (1H, brs).

3) 5-(Aminomethyl)-6-hydroxy-2-methyl-4-(4-methylphenyl)nicotinic acid hydrochloride (167 mg, yield 99%) was obtained as a white solid from tert-butyl 5-{[(tert-butoxycarbonyl)amino]methyl}-6-hydroxy-2-methyl-4-(4-methylphenyl)nicotinate (210 mg, 0.490 mmol) according to a method similar to the method of Example 24-1).

¹H-NMR (DMSO-d₆) δ:2.33 (3H, s), 2.35 (3H, s), 3.51 (2H, s), 7.15 (2H, d, J=7.9 Hz), 7.26 (2H, d, J=7.9 Hz), 7.94 (3H, brs), 12.42 (1H, s), 12.74 (1H, s).

### Example 113

### 5-(aminomethyl)-N,6-diisobutyl-2-methyl-4-(4-methylphenyl)nicotinamide ditrifluoroacetate

5-{[(tert-Butoxycarbonyl)amino]methyl}-6-isobutyl-2-methyl-4-(4-methylphenyl)nicotinic acid (23.9 mg, 0.06 mmol), isobutylamine (5.3 mg, 0.072 mmol), 1-hydroxy-1H-benzotriazole (11.0 mg, 0.072 mmol) and 1-ethyl-3-(3-dimethylaminopropyl)carbodiimide hydrochloride (13.8 mg, 0.072 mmol) were dissolved in a mixed solvent of N,N-dimethylformamide (1.25 mL)-dichloromethane (0.4 mL), and the mixture was stirred at 50° C. for 2 days. The reaction mixture was diluted with dichloromethane (3 mL) and washed successively with saturated aqueous sodium hydrogen carbonate (0.5 mL) and saturated brine (0.5 mL). Trifluoroacetic acid (2 mL) was added to the organic layer and the mixture was stirred for 2 hrs. The solvent was evaporated under reduced pressure and the residue was purified by preparative HPLC to give 5-(aminomethyl)-N,6-diisobutyl-2-methyl-4-(4-methylphenyl)nicotinamide ditrifluoroacetate (22.4 mg, yield 63%) as a yellow oil.

EIMS (M+1): 368

The compounds of Examples 114-168 were synthesized from nicotinic acids and amines corresponding to the following Tables 1-4 according to a method similar to the method of Example 113. The compounds of Examples 162-164 were obtained as free form by neutralizing the resulting trifluoroacetate of nicotinic amides with saturated aqueous sodium hydrogen carbonate.

TABLE 1

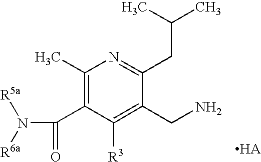

| Example | —NR^{5a}R^{6a} | —R³ | EIMS (M + 1) | HA |
|---|---|---|---|---|
| 113 | 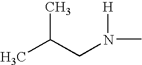 | 4-Me-Phenyl | 368 | 2CF₃COOH |
| 114 | 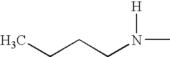 | 4-Me-Phenyl | 368 | 2CF₃COOH |
| 115 | 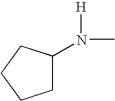 | 4-Me-Phenyl | 380 | 2CF₃COOH |
| 116 | 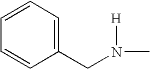 | 4-Me-Phenyl | 402 | 2CF₃COOH |
| 117 | 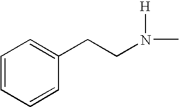 | 4-Me-Phenyl | 416 | 2CF₃COOH |
| 118 | 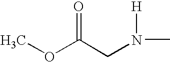 | 4-Me-Phenyl | 384 | 2CF₃COOH |
| 119 | 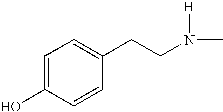 | 4-Me-Phenyl | 432 | 2CF₃COOH |
| 120 | 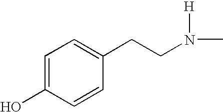 | 4-F-Phenyl | 436 | 2CF₃COOH |
| 121 | 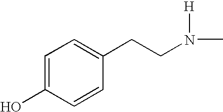 | 2,6-di-F- Phenyl | 454 | 2CF₃COOH |
| 122 | 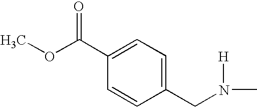 | 4-Me-Phenyl | 460 | 2CF₃COOH |
| 123 | 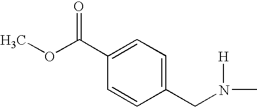 | 4-F-Phenyl | 464 | 2CF₃COOH |
| 124 | 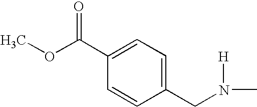 | 2,6-di-F- Phenyl | 482 | 2CF₃COOH |
| 125 | 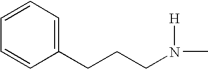 | 4-Me-Phenyl | 430 | 2CF₃COOH |
| 126 | 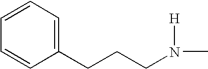 | 4-F-Phenyl | 434 | 2CF₃COOH |
| 127 | 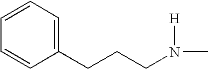 | 2,6-di-F- Phenyl | 452 | 2CF₃COOH |

TABLE 2

| | | | EIMS | |
|---|---|---|---|---|
| Example | —NR^{5a}R^{6a} | —R³ | (M + 1) | HA |
| 128 | 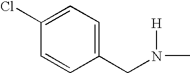 | 4-Me-Phenyl | 437 | 2CF₃COOH |
| 129 | 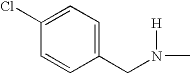 | 4-F-Phenyl | 440 | 2CF₃COOH |
| 130 | 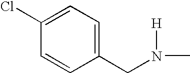 | 2,6-di-F- Phenyl | 458 | 2CF₃COOH |
| 131 | 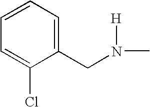 | 4-Me-Phenyl | 437 | 2CF₃COOH |
| 132 | 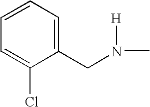 | 4-F-Phenyl | 440 | 2CF₃COOH |
| 133 | 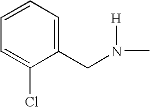 | 2,6-di-F- Phenyl | 458 | 2CF₃COOH |
| 134 | 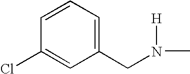 | 4-Me-Phenyl | 437 | 2CF₃COOH |
| 135 | 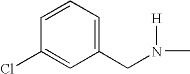 | 4-F-Phenyl | 440 | 2CF₃COOH |
| 136 | 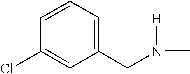 | 2,6-di-F- Phenyl | 458 | 2CF₃COOH |
| 137 | 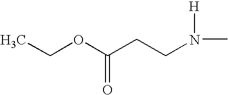 | 4-Me-Phenyl | 412 | 2CF₃COOH |
| 138 | 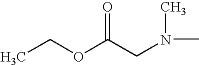 | 4-Me-Phenyl | 412 | 2CF₃COOH |
| 139 | 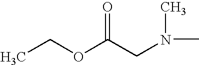 | 2,6-di-F- Phenyl | 434 | 2CF₃COOH |
| 140 | 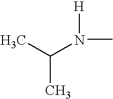 | 4-Me-Phenyl | 354 | 2CF₃COOH |
| 141 | 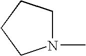 | 4-Me-Phenyl | 366 | 2CF₃COOH |
| 142 | 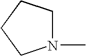 | 4-F-Phenyl | 370 | 2CF₃COOH |
| 143 | 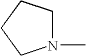 | 2,6-di-F- Phenyl | 388 | 2CF₃COOH |

TABLE 3

| | | | EIMS | |
|---|---|---|---|---|
| Example | —NR^{5a}R^{6a} | —R³ | (M + 1) | HA |
| 144 | 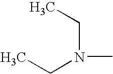 | 4-Me-Phenyl | 368 | 2CF₃COOH |
| 145 | 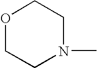 | 4-Me-Phenyl | 382 | 2CF₃COOH |
| 146 | 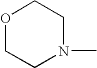 | 4-F-Phenyl | 386 | 2CF₃COOH |
| 147 | 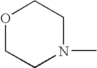 | 2,6-di-F- Phenyl | 404 | 2CF₃COOH |
| 148 | 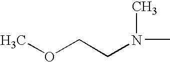 | 4-Me-Phenyl | 384 | 2CF₃COOH |
| 149 | 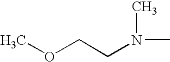 | 2,6-di-F- Phenyl | 406 | 2CF₃COOH |
| 150 | 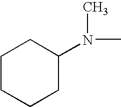 | 4-Me-Phenyl | 408 | 2CF₃COOH |
| 151 | 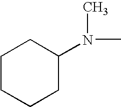 | 2,6-di-F- Phenyl | 430 | 2CF₃COOH |
| 152 | 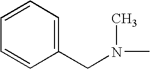 | 4-Me-Phenyl | 416 | 2CF₃COOH |
| 153 | 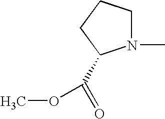 | 4-Me-Phenyl | 424 | 2CF₃COOH |
| 154 | 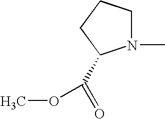 | 4-F-Phenyl | 428 | 2CF₃COOH |
| 155 | 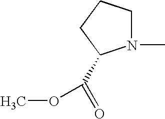 | 2,6-di-F- Phenyl | 446 | 2CF₃COOH |
| 156 | 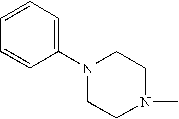 | 4-Me-Phenyl | 457 | 3CF₃COOH |
| 157 | 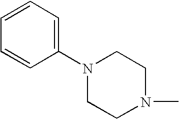 | 4-F-Phenyl | 461 | 3CF₃COOH |
| 158 | 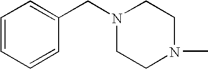 | 4-Me-Phenyl | 471 | 3CF₃COOH |

TABLE 4

| | | | EIMS | |
|---|---|---|---|---|
| Example | —NR^{5a}R^{6a} | —R³ | (M + 1) | HA |
| 159 | 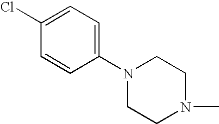 | 4-Me-Phenyl | 492 | 3CF₃COOH |
| 160 | 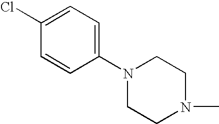 | 4-F-Phenyl | 496 | 3CF₃COOH |
| 161 | 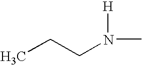 | 4-Me-Phenyl | 354 | 2CF₃COOH |
| 162 | 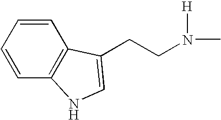 | 4-Me-Phenyl | 455 | |
| 163 | 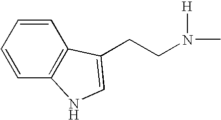 | 4-F-Phenyl | 459 | |
| 164 | 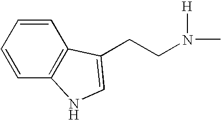 | 2,6-di-F- Phenyl | 477 | |
| 165 | 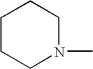 | 4-F-Phenyl | 384 | 2CF₃COOH |
| 166 | 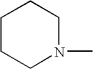 | 2,6-di-F- Phenyl | 402 | 2CF₃COOH |
| 167 | 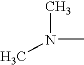 | 4-F-Phenyl | 344 | 2CF₃COOH |
| 168 | 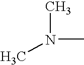 | 2,6-di-F- Phenyl | 362 | 2CF₃COOH |

### Example 169

### 4-(methoxycarbonyl)benzyl 5-(aminomethyl)-6-isobutyl-2-methyl-4-(4-methylphenyl)nicotinate dihydrochloride

1) To a solution (20 mL) of 5-{[(tert-butoxycarbonyl)amino]methyl}-6-isobutyl-2-methyl-4-(4-methylphenyl)nicotinic acid (2.00 g, 4.85 mmol) in N,N-dimethylformamide were added methyl 4-(bromomethyl)benzoate (1.22 g, 5.33 mmol) and potassium carbonate (1.01 g, 7.28 mmol) and the mixture was stirred at room temperature for 14 hrs. The reaction mixture was diluted with ethyl acetate (100 mL) and washed with saturated brine. The organic layer was dried over anhydrous magnesium sulfate and the solvent was evaporated under reduced pressure. The obtained residue was purified by silica gel column chromatography to give 4-(methoxycarbonyl)benzyl 5-{[(tert-butoxycarbonyl)amino]methyl}-6-isobutyl-2-methyl-4-(4-methylphenyl)nicotinate (2.50 g, yield 92%) as a colorless oil.

¹H-NMR (CDCl₃) δ:0.96 (6H, d, J=6.6 Hz), 1.38 (9H, s), 2.14-2.25 (1H, m), 2.35 (3H, s), 2.54 (3H, s), 2.78 (2H, d, J=7.2 Hz), 3.93 (3H, s), 4.12 (2H, d, J=7.0 Hz), 4.21 (1H, brs), 4.98 (2H, s), 7.01 (2H, d, J=7.9 Hz), 7.07-7.12 (4H, m), 7.93 (2H, d, J=8.3 Hz).

2) 4-(Methoxycarbonyl)benzyl 5-(aminomethyl)-6-isobutyl-2-methyl-4-(4-methylphenyl)nicotinate dihydrochloride (427 mg, yield 90%) was obtained as a white powder from 4-(methoxycarbonyl)benzyl 5-{[(tert-butoxycarbonyl)amino]methyl}-6-isobutyl-2-methyl-4-(4-methylphenyl)nicotinate (0.50 g, 0.892 mmol) according to a method similar to the method of Example 2-3).

¹H-NMR (DMSO-d₆) δ:0.96 (6H, d, J=6,8 Hz), 2.20 (1H, m), 2.34 (3H, s), 2.85 (2H, d, J=6.6 Hz), 3.80 (2H, d, J=5.3 Hz), 3.87 (3H, s), 5.07 (2H, s), 7.13-7.16 (4H, m), 7.20 (2H, d, J=7.9 Hz), 7.87 (2H, d, J=8.3 Hz), 8.22 (3H, brs).

### Example 170

### 4-[({[5-(aminomethyl)-6-isobutyl-2-methyl-4-(4-methylphenyl)pyridin-3-yl]carbonyl}oxy)methyl]benzoic acid dihydrochloride

1) 4-[({[5-{[(tert-Butoxycarbonyl)amino]methyl}-6-isobutyl-2-methyl-4-(4-methylphenyl)pyridin-3-yl]carbonyl}oxy)methyl]benzoic acid (340 mg, yield 32%) was obtained as a colorless oil from 4-(methoxycarbonyl)benzyl 5-{[(tert-butoxycarbonyl)amino]methyl}-6-isobutyl-2-methyl-4-(4-methylphenyl)nicotinate (1.10 g, 1.96 mmol) according to a method similar to the method of Example 9-1).

¹H-NMR (CDCl₃) δ:0.97 (6H, d, J=6.6 Hz), 1.39 (9H, s), 2.16-2.27 (1H, m), 2.35 (3H, s), 2.55 (3H, s), 2.79 (2H, d, J=7.4 Hz), 4.12 (2H, s), 4.22 (1H, brs), 5.00 (2H, s), 7.02 (2H, d, J=7.7 Hz), 7.06-7.14 (4H, m), 7.99 (2H, d, J=8.3 Hz).

2) 4-[({[5-(Aminomethyl)-6-isobutyl-2-methyl-4-(4-methylphenyl)pyridin-3-yl]carbonyl}oxy)methyl]benzoic acid dihydrochloride (326 mg, yield 93%) was obtained as a white powder from 4-[({[5-{[(tert-butoxycarbonyl)amino]methyl}-6-isobutyl-2-methyl-4-(4-methylphenyl)pyridin-3-yl]carbonyl}oxy)methyl]benzoic acid (370 mg, 0.677 mmol) according to a method similar to the method of Example 2-3).

¹H-NMR (DMSO-d₆) δ:0.95 (6H, d, J=6.6 Hz), 2.17-2.27 (1H, m), 2.34 (3H, s), 2.80 (2H, d, J=7.5 Hz), 3.80 (2H, d, J=5.8 Hz), 5.06 (2H, s), 7.10-7.14 (4H, m), 7.20 (2H, d, J=8.1 Hz), 8.10 (3H, brs).

### Example 171

### 2-amino-2-thioxoethyl 5-(aminomethyl)-6-isobutyl-2-methyl-4-(4-methylphenyl)nicotinate dihydrochloride

1) To a solution (50 mL) of 5-{[(tert-butoxycarbonyl)amino]methyl}-6-isobutyl-2-methyl-4-(4-methylphenyl)nicotinic acid (3.00 g, 7.27 mmol) in N,N-dimethylformamide were added bromoacetonitrile (0.66 mL, 9.45 mmol) and potassium carbonate (1.51 g, 10.9 mmol) and the mixture was stirred at room temperature for 1 hr. The reaction mixture was diluted with ethyl acetate (100 mL) and washed with saturated brine. The organic layer was dried over anhydrous magnesium sulfate and the solvent was evaporated under reduced pressure. The obtained residue was purified by silica gel column chromatography to give cyanomethyl 5-{[(tert-butoxycarbonyl)amino]methyl}-6-isobutyl-2-methyl-4-(4-methylphenyl)nicotinate (2.78 g, yield 85%) as a yellow solid.

¹H-NMR (CDCl₃) δ:0.98 (6H, d, J=6.6 Hz), 1.39 (9H, s), 2.19-2.28 (1H, m), 2.39 (3H, s), 2.56 (3H, s), 2.80 (2H, d, J=7.2 Hz), 4.17 (2H, d, J=4.9 Hz), 4.24 (1H, brs), 4.50 (2H, s), 7.05 (2H, d, J=8.1 Hz), 7.24 (2H, d, J=7.9 Hz).

2) Hydrogen sulfide was blown into a solution (25 mL) of cyanomethyl 5-{[(tert-butoxycarbonyl)amino]methyl}-6-isobutyl-2-methyl-4-(4-methylphenyl)nicotinate (2.78 g, 6.16 mmol) and triethylamine (0.94 mL, 6.77 mmol) in N,N-dimethylformamide for 1 hr. The solvent was evaporated under reduced pressure and the residue was diluted with ethyl acetate (100 mL). The solution was washed with saturated brine and dried over anhydrous magnesium sulfate. The solvent was evaporated under reduced pressure and the obtained yellow solid was washed with diisopropyl ether to give 2-amino-2-thioxoethyl 5-{[(tert-butoxycarbonyl)amino]methyl}-6-isobutyl-2-methyl-4-(4-methylphenyl)nicotinate (2.81 g, yield 94%) as a yellow brown solid.

¹H-NMR (CDCl₃) δ:0.98 (6H, d, J=6.6 Hz), 1.39 (9H, s), 2.19-2.28 (1H, m), 2.40 (3H, s), 2.56 (3H, s), 2.79 (2H, d, J=7.4 Hz), 4.14 (2H, d, J=4.5 Hz), 4.22 (1H, brs), 4.80 (2H, s), 6.21 (1H, brs), 6.98 (1H, brs), 7.13 (2H, d, J=7.9 Hz), 7.27 (2H, d, J=7.5 Hz).

3) 2-Amino-2-thioxoethyl 5-(aminomethyl)-6-isobutyl-2-methyl-4-(4-methylphenyl)nicotinate dihydrochloride (133 mg, yield 70%) was obtained as a yellow solid from 2-amino-2-thioxoethyl 5-{[(tert-butoxycarbonyl)amino]methyl}-6-isobutyl-2-methyl-4-(4-methylphenyl)nicotinate (200 mg, 0.412 mmol) according to a method similar to the method of Example 2-3).

¹H-NMR (DMSO-d₆) δ:0.97 (6H, d, J=6.6 Hz), 2.16-2.27 (1H, m), 2.37 (3H, s), 2.58 (3H, s), 2.83 (2H, d, J=6.2 Hz), 3.83 (2H, d, J=5.7 Hz), 4.45 (2H, s), 7.21 (2H, d, J=7.7 Hz), 7.29 (2H, d, J=7.9 Hz), 8.16 (3H, brs), 8.98 (1H, brs), 9.85 (1H, brs).

### Example 172

### [4-(ethoxycarbonyl)-1,3-thiazol-2-yl]methyl 5-(aminomethyl)-6-isobutyl-2-methyl-4-(4-methylphenyl)nicotinate dihydrochloride

1) To a mixed solution of 2-amino-2-thioxoethyl 5-(aminomethyl)-6-isobutyl-2-methyl-4-(4-methylphenyl)nicotinate dihydrochloride (2.02 g, 4.41 mmol) in tetrahydrofuran (30 mL)-saturated aqueous sodium hydrogen carbonate (10 mL) was added benzyl chloroformate (903 mg, 5.30 mmol) and the mixture was stirred at room temperature for 1 hr. The reaction mixture was diluted with ethyl acetate (100 mL) and washed with saturated brine. The organic layer was dried over anhydrous magnesium sulfate and the solvent was evaporated under reduced pressure. The obtained residue was purified by silica gel column chromatography to give 2-amino-2-thioxoethyl 5-({[(benzyloxy)carbonyl]amino}methyl)-6-isobutyl-2-methyl-4-(4-methylphenyl)nicotinate (2.00 g, yield 87%) as a pale-yellow solid.

¹H-NMR (CDCl₃) δ:0.97 (6H, d, J=6.6 Hz), 2.16-2.25 (1H, m), 2.39 (3H, s), 2.56 (3H, s), 2.81 (2H, d, J=7.4 Hz), 4.22 (2H, d, J=5.1 Hz), 4.43 (1H, brs), 4.79 (2H, s), 5.04 (2H, s), 6.23 (1H, brs), 6.97 (1H, brs), 7.11 (2H, d, J=8.1 Hz), 7.24 (2H, d, J=7.9 Hz), 7.29-7.36 (5H, m).

2) A solution (70 mL) of 2-amino-2-thioxoethyl 5-({[(benzyloxy)carbonyl]amino}methyl)-6-isobutyl-2-methyl-4-(4-methylphenyl)nicotinate (2.00 g, 3.85 mmol) and ethyl bromopyruvate (1.08 g, 5.00 mmol) in ethanol was heated under reflux for 1 hr. The reaction mixture was diluted with ethyl acetate (200 mL) and washed with saturated aqueous sodium hydrogen carbonate. The organic layer was dried over anhydrous magnesium sulfate. The solvent was evaporated under reduced pressure and the obtained residue was purified by silica gel column chromatography to give [4-(ethoxycarbonyl)-1,3-thiazol-2-yl]methyl 5-({[(benzyloxy)carbonyl]amino}methyl)-6-isobutyl-2-methyl-4-(4-methylphenyl)nicotinate (2.37 g, yield 100%) as a colorless oil.

¹H-NMR (CDCl₃) δ:0.96 (6H, d, J=6.6 Hz), 1.41 (3H, t, J=7.2 Hz), 2.10-2.26 (1H, m), 2.32 (3H, s), 2.56 (3H, s), 2.82 (2H, d, J=7.2 Hz), 4.21 (2H, d, J=5.3 Hz), 4.44 (2H, q, J=7.0 Hz), 5.03 (3H, s), 5.22 (2H, s), 7.00 (2H, d, J=8.1 Hz), 7.07 (2H, d, J=7.9 Hz), 7.22-7.38 (5H, m), 8.15 (1H, s).

3) [4-(Ethoxycarbonyl)-1,3-thiazol-2-yl]methyl 5-({[(benzyloxy)carbonyl]amino}methyl)-6-isobutyl-2-methyl-4-(4-methylphenyl)nicotinate (2.37 g, 3.85 mmol) was dissolved in 30% hydrogen bromide acetic acid solution (30 mL) and the mixture was stirred at room temperature for 30 min. The solvent was evaporated under reduced pressure and the obtained residue was dissolved by adding saturated aqueous sodium hydrogen carbonate (30 mL) and tetrahydrofuran (50 mL). Di-tert-butyl dicarbonate (1.02 g, 4.66 mmol) was added and the mixture was stirred at room temperature for 15 hrs. The reaction mixture was diluted with ethyl acetate (200 mL) and washed with saturated brine. The organic layer was dried over anhydrous magnesium sulfate. The solvent was evaporated under reduced pressure and the obtained residue was purified by silica gel column chromatography to give [4-(ethoxycarbonyl)-1,3-thiazol-2-yl]methyl 5-{[(tert-butoxycarbonyl)amino]methyl}-6-isobutyl-2-methyl-4-(4-methylphenyl)nicotinate (1.72 g, yield 78%) as a colorless oil.

¹H-NMR (CDCl₃) δ:0.97 (6H, d, J=6.6 Hz), 1.38 (9H, s), 1.42 (3H, t, J=7.2 Hz), 2.17-2.27 (1H, m), 2.33 (3H, s), 2.56 (3H, s), 2.79 (2H, d, J=7.4 Hz), 4.11-4.16 (2H, m), 4.24 (1H, brs), 4.44 (2H, q, J=7.2 Hz), 5.22 (2H, s), 7.02 (2H, d, J=8.1 Hz), 7.10 (2H, d, J=7.9 Hz), 8.16 (1H, s).

4) [4-(Ethoxycarbonyl)-1,3-thiazol-2-yl]methyl 5-(aminomethyl)-6-isobutyl-2-methyl-4-(4-methylphenyl)nicotinate dihydrochloride (322 mg, yield 90%) was obtained as a white powder from [4-(ethoxycarbonyl)-1,3-thiazol-2-yl]methyl 5-{[(tert-butoxycarbonyl)amino]methyl}-6-isobutyl-2-methyl-4-(4-methylphenyl)nicotinate (373 mg, 0.643 mmol) according to a method similar to the method of Example 2-3).

¹H-NMR (DMSO-d₆) δ:0.96 (6H, d, J=6.6 Hz), 1.32 (3H, t, J=7.2 Hz), 2.18-2.27 (1H, m), 2.29 (3H, s), 2.55 (3H, s), 2.80-2.92 (2H, m), 3.79 (2H, d, J=5.3 Hz), 4.32 (2H, q, J=7.1 Hz), 5.30 (2H, s), 7.12 (4H, s), 8.25 (3H, brs), 8.56 (1H, s).

### Example 173

### 2-[({[5-(aminomethyl)-6-isobutyl-2-methyl-4-(4-methylphenyl)pyridin-3-yl]carbonyl}oxy)methyl]-1,3-thiazole-4-carboxylic acid dihydrochloride

1) 2-[({[5-{[(tert-Butoxycarbonyl)amino]methyl}-6-isobutyl-2-methyl-4-(4-methylphenyl)pyridin-3-yl]carbonyl}oxy)methyl]-1,3-thiazole-4-carboxylic acid (1.21 g, yield 95%) was obtained as a colorless oil from [4-(ethoxycarbonyl)-1,3-thiazol-2-yl]methyl 5-{[(tert-butoxycarbonyl)amino]methyl}-6-isobutyl-2-methyl-4-(4-methylphenyl)nicotinate (1.34 g, 2.30 mmol) according to a method similar to the method of Example 9-1).

¹H-NMR (CDCl₃) δ:0.98 (6H, d, J=6.4 Hz), 1.38 (9H, s), 2.16-2.28 (1H, m), 2.33 (3H, s), 2.61 (3H, brs), 2.85 (2H, brs), 4.11-4.19 (2H, m), 4.23 (1H, brs), 5.22 (2H, s), 7.02 (2H, d, J =7.9 Hz), 7.10 (2H, d, J=7.4 Hz), 8.24 (1H, s). 2) 2-[({[5-(Aminomethyl)-6-isobutyl-2-methyl-4-(4-methylphenyl)pyridin-3-yl]carbonyl}oxy)methyl]-1,3-thiazole-4-carboxylic acid dihydrochloride (362 mg, yield 83%) was obtained as a pale-yellow powder from 2-[({[5-{[(tert-butoxycarbonyl)amino]methyl}-6-isobutyl-2-methyl-4-(4-methylphenyl)pyridin-3-yl]carbonyl}oxy)methyl]-1,3-thiazole-4-carboxylic acid (460 mg, 0.831 mmol) according to a method similar to the method of Example 2-3).

¹H-NMR (DMSO-d₆) δ:0.96 (6H, d, J=6.6 Hz), 2.16-2.27 (1H, m), 2.30 (3H, s), 2.53 (3H, s), 2.85 (2H, d, J=7.0 Hz), 3.80 (2H, d, J=5.1 Hz), 5.29 (2H, s), 7.12 (4H, s), 8.21 (3H, brs), 8.48 (1H, s).

### Example 174

### [4-(aminocarbonyl)-1,3-thiazol-2-yl]methyl 5-(aminomethyl)-6-isobutyl-2-methyl-4-(4-methylphenyl)nicotinate dihydrochloride

1) [4-(Aminocarbonyl)-1,3-thiazol-2-yl]methyl 5-{[(tert-butoxycarbonyl)amino]methyl}-6-isobutyl-2-methyl-4-(4-methylphenyl)nicotinate (420 mg, yield 70%) was obtained as a colorless oil from 2-[({[5-{[(tert-butoxycarbonyl)amino]methyl}-6-isobutyl-2-methyl-4-(4-methylphenyl)pyridin-3-yl]carbonyl}oxy)methyl]-1,3-thiazole-4-carboxylic acid (602 mg, 1.09 mmol) according to a method similar to the method of Example 3-1).

¹H-NMR (CDCl₃) δ:0.97 (6H, d, J=6.6 Hz), 1.38 (9H, s), 2.18-2.27 (1H, m), 2.33 (3H, s), 2.57 (3H, s), 2;79 (2H, d, J=7.4 Hz), 4.10-4.16 (2H, m), 4.22 (1H, brs), 5.17 (2H, s), 5.64 (1H, brs), 7.01 (2H, d, J=7.9 Hz), 7.09 (2H, d, J=7.9 Hz), 8.13 (1H, s).

2) [4-(Aminocarbonyl)-1,3-thiazol-2-yl]methyl 5-(aminomethyl)-6-isobutyl-2-methyl-4-(4-methylphenyl)nicotinate dihydrochloride (208 mg, yield 48%) was obtained as a white powder from [4-(aminocarbonyl)-1,3-thiazol-2-yl]methyl 5-{[(tert-butoxycarbonyl)amino]methyl}-6-isobutyl-2-methyl-4-(4-methylphenyl)nicotinate (460 mg, 0.832 mmol) according to a method similar to the method of Example 2-3).

¹H-NMR (DMSO-d₆) δ:0.96 (6H, d, J=6.6 Hz), 2.18-2.27 (1H, m), 2.30 (3H, s), 2.53 (3H, s), 2.79-2.89 (2H, m), 3.79 (2H, d, J=5.5 Hz), 5.28 (2H, s), 7.12 (4H, s), 7.62 (1H, brs), 7.66 (1H, brs), 8.22 (3H, brs), 8.48 (1H, s).

### Example 175

### [(2,2-dimethylpropanoyl)oxy]methyl 5-(aminomethyl)-6-isobutyl-2-methyl-4-(4-methylphenyl)nicotinate dihydrochloride

1) To a solution (20 mL) of 5-{[(tert-butoxycarbonyl)amino]methyl}-6-isobutyl-2-methyl-4-(4-methylphenyl)nicotinic acid (1.50 g, 3.37 mmol) in N,N-dimethylformamide were added chloromethyl pivalate (0.59 mL, 4.04 mmol) and potassium carbonate (0.93 g, 6.72 mmol) and the mixture was stirred at room temperature for 1 hr. The reaction mixture was diluted with ethyl acetate (100 mL) and washed with saturated brine. The organic layer was dried over anhydrous magnesium sulfate. The solvent was evaporated under reduced pressure and the obtained residue was purified by silica gel column chromatography to give [(2,2-dimethylpropanoyl)oxy]methyl 5-{[(tert-butoxycarbonyl)amino]methyl}-6-isobutyl-2-methyl-4-(4-methylphenyl)nicotinate (1.68 g, yield 95%) as a yellow oil.

¹H-NMR (CDCl₃) δ:0.97 (6H, d, J=6.6 Hz), 1.16 (9H, s), 1.39 (9H, s), 2.14-2.29 (1H, m), 2.38 (3H, s), 2.54 (3H, s), 2.78 (2H, d, J=7.4 Hz), 4.13 (2H, d, J=4.9 Hz), 4.21 (1H, brs), 5.57 (2H, s), 7.06 (2H, d, J=8.1 Hz), 7.20 (2H, d, J=7.9 Hz).

2) [(2,2-Dimethylpropanoyl)oxy]methyl 5-(aminomethyl)-6-isobutyl-2-methyl-4-(4-methylphenyl)nicotinate dihydrochloride (1.58 g , yield 99%) was obtained as a white solid from [(2,2-dimethylpropanoyl)oxy]methyl 5-{[(tert-butoxycarbonyl)amino]methyl}-6-isobutyl-2-methyl-4-(4-methylphenyl)nicotinate (1.68 g, 3.19 mmol) according to a method similar to the method of Example 2-3).

¹H-NMR (DMSO-d₆) δ:0.96 (6H, d, J=6.6 Hz), 1.09 (9H, s), 2.17-2.29 (1H, m), 2.37 (3H, s), 2.49 (3H, s), 2.84 (2H, d, J=7.0 Hz), 3.78 (2H, d, J=5.5 Hz), 5.61 (2H, s), 7.19 (2H, d, J=8.1 Hz), 7.28 (2H, d, J=8.1 Hz), 8.20 (3H, brs).

### Example 176

### (5-methyl-2-oxo-1,3-dioxol-4-yl)methyl 5-(aminomethyl)-6-isobutyl-2-methyl-4-(4-methylphenyl)nicotinate dihydrochloride

1) To a solution (20 mL) of 5-{[(tert-butoxycarbonyl)amino]methyl}-6-isobutyl-2-methyl-4-(4-methylphenyl)nicotinic acid (1.50 g, 3.37 mmol) in N,N-dimethylformamide were added 4-(chloromethyl)-5-methyl-1,3-dioxol-2-one (0.60 g, 4.04 mmol) and potassium carbonate (0.93 g, 6.72 mmol) and the mixture was stirred at room temperature for 1 hr. The reaction mixture was diluted with ethyl acetate (100 mL) and the mixture was washed with saturated brine. The organic layer was dried over anhydrous magnesium sulfate. The solvent was evaporated under reduced pressure and the obtained residue was purified by silica gel column chromatography to give (5-methyl-2-oxo-1,3-dioxol-4-yl)methyl 5-{[(tert-butoxycarbonyl)amino]methyl}-6-isobutyl-2-methyl-4-(4-methylphenyl)nicotinate (1.50 g, yield 85%) as a colorless oil.

¹H-NMR (CDCl₃) δ:0.97 (6H, d, J=6.8 Hz), 1.38 (9H, s), 1.97 (3H, s), 2.16-2.26 (1H, m), 2.40 (3H, s), 2.54 (3H, s), 2.79 (2H, d, J=7.4 Hz), 4.09 (2H, s), 4.74 (2H, s), 7.10 (2H, d, J=7.9 Hz), 7.17 (2H, d, J=7.9 Hz).

2) (5-Methyl-2-oxo-1,3-dioxol-4-yl)methyl 5-(aminomethyl)-6-isobutyl-2-methyl-4-(4-methylphenyl)nicotinate dihydrochloride (1.21 g, yield 85%) was obtained as a white powder from (5-methyl-2-oxo-1,3-dioxol-4-yl)methyl 5-{[(tert-butoxycarbonyl)amino]methyl}-6-isobutyl-2-methyl-4-(4-methylphenyl)nicotinate (1.50 g, 2.86 mmol) according to a method similar to the method of Example 2-3).

¹H-NMR (DMSO-d₆) δ:0.96 (6H, d, J=6.6 Hz), 1.97 (3H, s), 2.17-2.28 (1H, m), 2.35 (3H; s), 2.82 (2H, d, J=7.0 Hz), 3.79 (2H, d, J=5.5 Hz), 4.93 (2H, s), 7.12 (2H, d, J=8.1 Hz), 7.20 (2H, d, J=7.9 Hz), 8.15 (3H, brs).

### Example 177

### 3-oxo-1,3-dihydro-2-benzofuran-1-yl 5-(aminomethyl)-6-isobutyl-2-methyl-4-(4-methylphenyl)nicotinate dihydrochloride

1) To a solution (30 mL) of 5-{[(tert-butoxycarbonyl)amino]methyl}-6-isobutyl-2-methyl-4-(4-methylphenyl)nicotinic acid (1.50 g, 3.37 mmol) in N,N-dimethylformamide were added 3-chloro-2-benzofuran-1(3H)-one (0.86 g, 4.04 mmol) and potassium carbonate (0.93 g, 6.72 mmol) and the mixture was stirred at room temperature for 1 hr. The reaction mixture was diluted with ethyl acetate (100 mL) and the mixture was washed with saturated brine. The organic layer was dried over anhydrous magnesium sulfate. The solvent was evaporated under reduced pressure and the obtained residue was purified by silica gel column chromatography to give 3-oxo-1,3-dihydro-2-benzofuran-1-yl 5-{[(tert-butoxycarbonyl)amino]methyl}-6-isobutyl-2-methyl-4-(4-methylphenyl)nicotinate (1.83 g, yield 99%) as a colorless oil.

¹H-NMR (CDCl₃) δ:0.96 (6H, d, J=6.6 Hz), 1.38 (9H, s), 2.16-2.25 (1H, m), 2.42 (3H, s), 2.63 (3H, s), 2.78 (2H, d, J=7.4 Hz), 4.12 (2H, s), 6.98-7.08 (3H, m), 7.17 (2H, d, J=7.9 Hz), 7.24 (1H, s), 7.59-7.64 (2H, m), 7.83-7.88 (1H, m).

2) 3-Oxo-1,3-dihydro-2-benzofuran-1-yl 5-(aminomethyl)-6-isobutyl-2-methyl-4-(4-methylphenyl)nicotinate dihydrochloride was obtained as a white powder from 3-oxo-1,3-dihydro-2-benzofuran-1-yl 5-{[(tert-butoxycarbonyl)amino]methyl}-6-isobutyl-2-methyl-4-(4-methylphenyl)nicotinate (1.83 g, 3.36 mmol) according to a method similar to the method of Example 2-3).

¹H-NMR (DMSO-d₆) δ:0.95 (6H, d, J=6.6 Hz), 2.15-2.28 (1H, m), 2.38 (3H, s), 2.59 (3H, s), 2.81 (2H, d, J=7.2 Hz), 3.79 (2H, d, J=5.7 Hz), 7.07-7.15 (3H, m), 7.25-7.32 (2H, m), 7.40 (1H, s), 7.73-7.75 (1H, m), 7.79-7.84 (1H, m), 7.89 (1H, d, J=7.5 Hz), 8.12 (3H, brs).

### Example 178

### (2E)-2-(3-oxo-2-benzofuran-1(3H)-ylidene)ethyl 5-(aminomethyl)-6-isobutyl-2-methyl-4-(4-methylphenyl)nicotinate dihydrochloride

1) To a solution (10 mL) of 5-{[(tert-butoxycarbonyl)amino]methyl}-6-isobutyl-2-methyl-4-(4-methylphenyl)nicotinic acid (380 mg, 0.853 mmol) in N,N-dimethylformamide were added (3E)-3-(2-chloroethylidene)-2-benzofuran-1(3H)-one (170 mg, 0.711 mmol) and potassium carbonate (147 mg, 1.07 mmol) and the mixture was stirred at room temperature for 1 hr. The reaction mixture was diluted with ethyl acetate (100 mL) and the mixture was washed with saturated brine. The organic layer was dried over anhydrous magnesium sulfate. The solvent was evaporated under reduced pressure and the obtained residue was purified by silica gel column chromatography to give (2E)-2-(3-oxo-2-benzofuran-1(3H)-ylidene)ethyl 5-{[(tert-butoxycarbonyl)amino]methyl}-6-isobutyl-2-methyl-4-(4-methylphenyl)nicotinate (270 mg, yield 55%) as a colorless oil.

¹H-NMR (CDCl₃) δ:0.97 (6H, d, J=6.6 Hz), 1.38 (9H, s), 2.16-2.26 (4H, m), 2.58 (3H, s), 2.78 (2H, d, J=7.4 Hz), 4.12 (2H, s), 4.21 (1H, brs), 4.85 (2H, d, J=7.4 Hz), 5.25 (1H, t, J=7.4 Hz), 7.07 (2H, d, J=8.3 Hz), 7.12 (2H, d, J=8.1 Hz), 7.55-7.64 (2H, m), 7.72-7.78 (1H, m), 7.92-7.95 (1H, m). 2) (2E)-2-(3-Oxo-2-benzofuran-1(3H)-ylidene)ethyl 5-(aminomethyl)-6-isobutyl-2-methyl-4-(4-methylphenyl)nicotinate dihydrochloride (204 mg, yield 79%) was obtained as a white powder from (2E)-2-(3-oxo-2-benzofuran-1(3H)-ylidene)ethyl 5-{[(tert-butoxycarbonyl)amino]methyl}-6-isobutyl-2-methyl-4-(4-methylphenyl)nicotinate (270 mg, 0.473 mmol) according to a method similar to the method of Example 2-3).

¹H-NMR (DMSO-d₆) δ:0.95 (6H, d, J=6.6 Hz), 2.07 (3H, s), 2.18-2.29 (1H, m), 2.79 (2H, d, J=6.6 Hz), 3.78 (2H, d, J=7.4 Hz), 4.81 (2H, d, J=7.5 Hz), 5.68 (1H, t, J=7.5 Hz), 7.14 (4H, s), 7.71-7.77 (1H, m), 7.90-8.00 (3H, m), 8.06 (3H, brs).

### Example 179

### benzyl 5-(aminomethyl)-6-isobutyl-2-methyl-4-(4-methylphenyl)nicotinate

To a solution (30 mL) of 5-{[(tert-butoxycarbonyl)amino]methyl}-6-isobutyl-2-methyl-4-(4-methylphenyl)nicotinic acid (3.00 g, 6.73 mmol) in N,N-dimethylformamide were added benzyl bromide (0.80 mL, 6.73 mmol) and potassium carbonate (1.85 g, 13.4 mmol) and the mixture was stirred at room temperature for 1 hr. The reaction mixture was diluted with ethyl acetate (200 mL) and the mixture was washed with saturated brine. The organic layer was dried over anhydrous magnesium sulfate and the solvent was evaporated under reduced pressure. The obtained residue was dissolved in trifluoroacetic acid (50 mL) and the mixture was stirred at room temperature for 3 hrs. Trifluoroacetic acid was evaporated under reduced pressure, and the residue was neutralized with saturated aqueous sodium hydrogen carbonate. The mixture was extracted with ethyl acetate. The organic layer was washed with saturated brine and dried over anhydrous magnesium sulfate. The solvent was evaporated under reduced pressure and the obtained residue was purified by silica gel column chromatography to give benzyl 5-(aminomethyl)-6-isobutyl-2-methyl-4-(4-methylphenyl)nicotinate (2.70 g, yield 99%) as a yellow solid.

¹H-NMR (CDCl₃) δ:0.91 (6H, d, J=6.6 Hz), 2.07-2.18 (1H, m), 2.34 (3H, s), 2.51 (3H, s), 2.72 (2H, d, J=7.4 Hz), 3.84 (2H, s), 4.94 (2H, s), 7.02-7.12 (6H, m), 7.24-7.31 (3H, m).

### Example 180

### 2-oxo-1,3-dioxolan-4-yl 5-(aminomethyl)-6-isobutyl-2-methyl-4-(4-methylphenyl)nicotinate dihydrochloride

1) To a solution (30 mL) of 5-{[(tert-butoxycarbonyl)amino]methyl}-6-isobutyl-2-methyl-4-(4-methylphenyl)nicotinic acid (1.50 g, 3.37 mmol) in N,N-dimethylformamide were added 4-chloro-1,3-dioxolan-2-one (0.55 g, 4.04 mmol) and potassium carbonate (0.70 g, 5.05 mmol) and the mixture was stirred at room temperature for 1 hr. The reaction mixture was diluted with ethyl acetate (100 mL) and the mixture was washed with saturated brine. The organic layer was dried over anhydrous magnesium sulfate. The solvent was evaporated under reduced pressure and the obtained residue was purified by silica gel column chromatography to give 2-oxo-1,3-dioxolan-4-yl 5-{[(tert-butoxycarbonyl)amino]methyl}-6-isobutyl-2-methyl-4-(4-methylphenyl)nicotinate (1.39 g, yield 83%) as a colorless oil.

¹H-NMR (CDCl₃) δ:0.98 (6H, d, J=6.8 Hz), 1.39 (9H, S), 2.19-2.28 (1H, m), 2.41 (3H, s), 2.60 (3H, s), 2.81 (2H, d, J=7.4 Hz), 3.67 (1H, dd, J=10.2, 1.5 Hz), 4.16 (2H, d, J=4.9 Hz), 4.22 (1H, brs), 4.31 (1H, dd, J=10.0, 5.7 Hz), 4.63-4.82 (1H, m), 6.41-6.46 (1H, m), 7.01-7.10 (2H, m), 7.19-7.26 (2H, m).

2) 2-Oxo-1,3-dioxolan-4-yl 5-(aminomethyl)-6-isobutyl-2-methyl-4-(4-methylphenyl)nicotinate dihydrochloride (1.31 g, yield 99%) was obtained as a white powder from 2-oxo-1,3-dioxolan-4-yl 5-{[(tert-butoxycarbonyl)amino]methyl}-6-isobutyl-2-methyl-4-(4-methylphenyl)nicotinate (1.39 g, 2.79 mmol) according to a method similar to the method of Example 2-3).

¹H-NMR (DMSO-d₆) δ:0.96 (6H, d, J=6.6 Hz), 2.18-2.28 (1H, m), 2.36 (3H, s), 2.55 (3H, s), 2.85 (2H, d, J=7.0 Hz), 3.83 (2H, d, J=5.7 Hz), 4.04 (1H, dd, J=10.2, 1.7 Hz), 4.59 (1H, dd, J=10.1, 5.7 Hz), 6.59 (1H, dd, J=5.4 Hz), 7.14-7.20 (2H, m), 7.24-7.29 (2H, m), 8.23 (3H, brs).

### Example 181

### 5-(aminomethyl)-4-(4-hydroxyphenyl)-6-isobutyl-2-methylnicotinic acid dihydrochloride

1) tert-Butyl 4-[4-(benzyloxy)phenyl]-5-cyano-6-isobutyl-2-methyl-1,4-dihydropyridine-3-carboxylate (21.4 g, yield 77%) was obtained as pale-pink solid from 4-(benzyloxy)benzaldehyde (12.8 g, 60.4 mmol) according to a method similar to the method of Example 1-2).

¹H-NMR (CDCl₃) δ:0.94 (3H, d, J=6.6 Hz), 0.99 (3H, d, J=6.6 Hz), 1.28 (9H, s), 1.80-1.96 (1H, m), 2.14-2.29 (2H, m), 2.32 (3H, s), 4.51 (1H, s), 5.03 (2H, s), 5.49 (1H, s), 6.90 (2H, d, J=8.7 Hz), 7.15 (2H, d, J=8.7 Hz), 7.29-7.46 (5H, m).

2) tert-Butyl 4-[4-(benzyloxy)phenyl]-5-cyano-6-isobutyl-2-methylnicotinate (2.18 g, yield 94%) was obtained as a yellow solid from tert-butyl 4-[4-(benzyloxy)phenyl]-5-cyano-6-isobutyl-2-methyl-1,4-dihydropyridine-3-carboxylate (2.33 g, 5.08 mmol) according to a method similar to the method of Example 23-3).

¹H-NMR (CDCl₃) δ:1.01 (6H, d, J=6.6 Hz), 1.25 (9H, S), 2.17-2.33 (1H, m), 2.63 (3H, s), 2.93 (2H, d, J=7.4 Hz), 5.12 (2H, s), 7.06 (2H, d, J=8.9 Hz), 7.31 (2H, d, J=8.9 Hz), 7.39-7.49 (5H, m).

3) tert-Butyl 5-(aminomethyl)-4-(4-hydroxyphenyl)-6-isobutyl-2-methylnicotinate was obtained as a crude product from tert-butyl 4-[4-(benzyloxy)phenyl]-5-cyano-6-isobutyl-2-methylnicotinate (2.13 g, 4.67 mmol) according to a method similar to the method of Example 1-4). tert-Butyl 5-{[(tert-butoxycarbonyl)amino]methyl}-4-(4-hydroxyphenyl)-6-isobutyl-2-methylnicotinate (1.35 g, yield 61%) was obtained as a pale-yellow solid from the crude product according to a method similar to the method of Example 2-1).

¹H-NMR (CDCl₃) δ:0.97 (6H, d, J=6.6 Hz), 1.22 (9H, s), 1.40 (9H, s), 2.12-2.27 (1H, m), 2.55 (3H, s), 2.76 (2H, d, J=7.2 Hz), 4.14 (2H, d, J=4.9 Hz), 4.25 (1H, brs), 5.50 (1H, brs), 6.85 (2H, d, J=8.5 Hz), 7.07 (2H, d, J=8.5 Hz).

4) tert-Butyl 5-{[(tert-butoxycarbonyl)amino]methyl}-4-(4-hydroxyphenyl)-6-isobutyl-2-methylnicotinate (316 mg, 0.671 mmol) and anisole (218 mg, 2.01 mmol) were dissolved in trifluoroacetic acid (5 mL) and the mixture was stirred at room temperature for 5 hrs. Trifluoroacetic acid was evaporated under reduced pressure and 4N hydrogen chloride 1,4-dioxane solution (20 mL) was added to the residue. The mixture was stirred at room temperature for 30 min. The solvent was evaporated under reduced pressure and the obtained yellow solid was washed with diisopropyl ether to give 5-(aminomethyl)-4-(4-hydroxyphenyl)-6-isobutyl-2-methylnicotinic acid dihydrochloride (259 mg, yield 99%) as a yellow powder.

¹H-NMR (DMSO-d₆) δ:0.97 (6H, d, J=6.6 Hz), 2.14-2.27 (1H, m), 2.59 (3H, s), 2.92 (2H, d, J=5.7 Hz), 3.86 (2H, d, J=4.9 Hz), 6.87 (2H, d, J=8.5 Hz), 7.14 (2H, d, J=8.3 Hz), 8.26 (3H, brs).

### Example 182

### 5-(aminomethyl)-6-isobutyl-4-(4-methoxyphenyl)-2-methylnicotinic acid dihydrochloride

1) To a solution (20 mL) of tert-butyl 5-{[(tert-butoxycarbonyl)amino]methyl}-4-(4-hydroxyphenyl)-6-isobutyl-2-methylnicotinate (620 mg, 1.32 mmol) and potassium carbonate (365 mg, 2.64 mmol) in N,N-dimethylformamide was added iodomethane (374 mg, 2.64 mmol) and the mixture was stirred at room temperature for 30 min. The reaction mixture was diluted with ethyl acetate (100 mL) and the mixture was washed with saturated brine. The organic layer was dried over anhydrous magnesium sulfate. The solvent was evaporated under reduced pressure and the obtained residue was purified by silica gel column chromatography to give tert-butyl 5-{[(tert-butoxycarbonyl)amino]methyl}-6-isobutyl-4-(4-methoxyphenyl)-2-methylnicotinate (520 mg, yield 81%) as a colorless oil.

¹H-NMR (CDCl₃) δ:0.97 (6H, d, J=6.8 Hz), 1.21 (9H, s), 1.39 (9H, s), 2.13-2.26 (1H, m), 2.55 (3H, s), 2.76 (2H, d, J=7.4 Hz), 3.84 (3H, s), 4.12 (2H, s), 4.22 (1H, brs), 6.94 (2H, d, J=8.7 Hz), 7.12 (2H, d, J=8.7 Hz).

2) 5-(Aminomethyl)-6-isobutyl-4-(4-methoxyphenyl)-2-methylnicotinic acid dihydrochloride (429 mg, yield 99%) was obtained as a yellow powder from tert-butyl 5-{[(tert-butoxycarbonyl)amino]methyl}-6-isobutyl-4-(4-methoxyphenyl)-2-methylnicotinate (520 mg, 1.07 mmol) according to a method similar to the method of Example 181-4).

¹H-NMR (DMSO-d₆) δ:0.97 (6H, d, J=6.6 Hz), 2.16-2.27 (1H, m), 2.54 (3H, s), 2.85 (2H, d, J=6.6 Hz), 3.57(3H, s), 3.84 (2H, s), 7.05 (2H, d, J=8.7 Hz), 7.26 (2H, d, J=8.7 Hz), 8.17 (3H, brs).

### Example 183

### methyl 4-({[5-(aminomethyl)-6-isobutyl-2-methyl-4-(4-methylphenyl)pyridin-3-yl]methyl}thio)benzoate dihydrochloride

1) A mixture of tert-butyl {[5-(hydroxymethyl)-2-isobutyl-6-methyl-4-(4-methylphenyl)pyridin-3-yl]methyl}carbamate (1.00 g, 2.51 mmol), triethylamine (0.7 mL, 5.02 mmol) and tetrahydrofuran (20 mL) was cooled to 0° C. and methanesulfonyl chloride (432 mg, 3.77 mmol) was added dropwise. After stirring at room temperature for 30 min., the reaction mixture was poured into saturated aqueous sodium hydrogen carbonate, and the mixture was extracted with ethyl acetate. The extract was dried over anhydrous magnesium sulfate and the solvent was evaporated under reduced pressure to give [5-{[(tert-butoxycarbonyl)amino]methyl}-6-isobutyl-2-methyl-4-(4-methylphenyl)pyridin-3-yl]methyl methanesulfonate as a crude product. The crude product was dissolved in N,N-dimethylformamide (15 mL), and potassium carbonate (520 mg, 3.77 mmol) and methyl 4-mercaptobenzoate (422 mg, 2.51 mmol) were added. The mixture was stirred with heating at 50° C. for 1 hr. The reaction mixture was diluted with ethyl acetate (100 mL) and the mixture was washed with saturated brine. The organic layer was dried over anhydrous magnesium sulfate. The solvent was evaporated under reduced pressure and the obtained residue was purified by silica gel column chromatography to give methyl 4-({[5-{[(tert-butoxycarbonyl)amino]methyl}-6-isobutyl-2-methyl-4-(4-methylphenyl)pyridin-3-yl]methyl}thio)benzoate (1.01 g, yield 73%) as a colorless oil.

¹H-NMR (CDCl₃) δ:0.97 (6H, d, J=6.6 Hz), 1.38 (9H, s), 2.16-2.25 (1H, m), 2.37 (3H, s), 2.65 (3H, s), 2.75 (2H, d, J=7.4 Hz), 3.86 (2H, s), 3.89 (3H, s), 4.04 (2H, d, J=5.1 Hz), 4.20 (1H, brs), 7.04 (2H, d, J=7.9 Hz), 7.09 (2H, d, J=8.7 Hz), 7.19 (2H, d, J=7.7 Hz), 7.85 (2H, d, J=8.7 Hz).

2) Methyl 4-({[5-(aminomethyl)-6-isobutyl-2-methyl-4-(4-methylphenyl)pyridin-3-yl]methyl}thio)benzoate dihydrochloride (138 mg, yield 73%) was obtained as a pale-yellow powder from methyl 4-({[5-{[(tert-butoxycarbonyl)amino]methyl}-6-isobutyl-2-methyl-4-(4-methylphenyl)pyridin-3-yl]methyl}thio)benzoate (200 mg, 0.365 mmol) according to a method similar to the method of Example 2-3).

¹H-NMR (DMSO-d₆) δ:0.98 (6H, d, J=6.6 Hz), 2.12-2.23 (1H, m), 2.35 (3H, s), 2.81 (3H, s), 3.64 (2H, brs), 3.75 (2H, d, J=5.7 Hz), 3.83 (3H, s), 4.01 (2H, s), 7.24-7.33 (6H, m), 7.82 (2H, d, J=8.7 Hz), 8.30 (3H, brs).

### Example 184

### 4-({[5-(aminomethyl)-6-isobutyl-2-methyl-4-(4-methylphenyl)pyridin-3-yl]methyl}thio)benzoic acid dihydrochloride

1) 4-({[5-{[(tert-Butoxycarbonyl)amino]methyl}-6-isobutyl-2-methyl-4-(4-methylphenyl)pyridin-3-yl]methyl}thio)benzoic acid (0.97 g, yield 72%) was obtained as a white solid from methyl 4-({[5-{[(tert-butoxycarbonyl)amino]methyl}-6-isobutyl-2-methyl-4-(4-methylphenyl)pyridin-3-yl]methyl}thio)benzoate (1.37 g, 2.51 mmol) according to a method similar to the method of Example 9-1).

¹H-NMR (CDCl₃) δ:1.07 (6H, d, J=6.6 Hz), 1.38 (9H, s), 2.23-2.35 (1H, m), 2.42 (3H, s), 3.08 (3H, s), 3.30-3.40 (2H, m), 3.90 (2H, s), 4.12-4.18 (2H, m), 4.30 (1H, brs), 7.05 (2H, d, J=7.9 Hz), 7.13 (2H, d, J=8.5 Hz), 7.23-7.31 (2H, m), 7.93 (2H, d, J=8.5 Hz).

2) 4-({[5-(Aminomethyl)-6-isobutyl-2-methyl-4-(4-methylphenyl)pyridin-3-yl]methyl}thio)benzoic acid dihydrochloride (198 mg, yield 77%) was obtained as a white powder from 4-({[5-{[(tert-butoxycarbonyl)amino]methyl}-6-isobutyl-2-methyl-4-(4-methylphenyl)pyridin-3-yl]methyl}thio)benzoic acid (0.27 g, 0.505 mmol) according to a method similar to the method of Example 2-3).

¹H-NMR (DMSO-d₆) δ:0.98 (6H, d, J=6.6 Hz), 2.13-2.23 (1H, m), 2.36 (3H, s), 2.81 (3H, s), 3.05 (2H, brs), 3.71-3.80 (2H, m), 4.01 (2H, s), 7.23-7.27 (4H, m), 7.32 (2H, d, J=8.1 Hz), 7.80 (2H, d, J=8.3 Hz), 8.32 (3H, brs).

### Example 185

### methyl 4-({[5-(aminomethyl)-6-isobutyl-2-methyl-4-(4-methylphenyl)pyridin-3-yl]methyl}sulfonyl)benzoate dihydrochloride

1) Methyl 4-({[5-{[(tert-butoxycarbonyl)amino]methyl}-6-isobutyl-2-methyl-4-(4-methylphenyl)pyridin-3-yl]methyl}sulfonyl)benzoate (410 mg, yield 84%) was obtained as a colorless oil from methyl 4-({[5-{[(tert-butoxycarbonyl)amino]methyl}-6-isobutyl-2-methyl-4-(4-methylphenyl)pyridin-3-yl]methyl}thio)benzoate (0.46 g, 0.838 mmol) according to a method similar to the method of Example 91-1).

¹H-NMR (CDCl₃) δ:0.98 (6H, d, J=6.7 Hz), 1.38 (9H, s), 2.17-2.26 (1H, m), 2.41 (3H, s), 2.64 (3H, s), 2.77 (2H, d, J=7.4 Hz), 3.98 (3H, s), 4.00 (2H, d, J=5.3 Hz), 4.18 (1H, brs), 4.32 (2H, s), 6.87 (2H, d, J=7.7 Hz), 7.17 (2H, d, J=7.7 Hz), 7.56 (2H, d, J=8.5 Hz), 8.08 (2H, d, J=8.5 Hz).

2) Methyl 4-({[5-(aminomethyl)-6-isobutyl-2-methyl-4-(4-methylphenyl)pyridin-3-yl]methyl}sulfonyl)benzoate dihydrochloride (352 mg, yield 90%) was obtained as a pale-yellow powder from methyl 4-({[5-{[(tert-butoxycarbonyl)amino]methyl}-6-isobutyl-2-methyl-4-(4-methylphenyl)pyridin-3-yl]methyl}sulfonyl)benzoate (410 mg, 0.706 mmol) according to a method similar to the method of Example 2-3).

¹H-NMR (DMSO-d₆) δ:0.98 (6H, d, J=6.6 Hz), 2.17-2.27 (1H, m), 2.38 (3H, s), 2.78 (3H, s), 3.00 (2H, brs), 3.66-3.74 (2H, m), 3.93 (3H, s), 4.61 (2H, brs), 7.05 (2H, d, J=7.9 Hz), 7.23 (2H, d, J=7.9 Hz), 7.66 (2H, d, J=8.3 Hz), 8.09 (2H, d, J=8.7 Hz), 8.30 (3H, brs).

Example 186

### 4-({[5-(aminomethyl)-6-isobutyl-2-methyl-4-(4-methylphenyl)pyridin-3-yl]methyl}sulfonyl)benzoic acid dihydrochloride

1) 4-({[5-{[(tert-Butoxycarbonyl)amino]methyl}-6-isobutyl-2-methyl-4-(4-methylphenyl)pyridin-3-yl]methyl}sulfonyl)benzoic acid (300 mg, yield 93%) was obtained as a colorless oil from methyl 4-({[5-{[(tert-butoxycarbonyl)amino]methyl}-6-isobutyl-2-methyl-4-(4-methylphenyl)pyridin-3-yl]methyl}sulfonyl)benzoate (330 mg, 0.568 mmol) according to a method similar to the method of Example 9-1).

¹H-NMR (CDCl₃) δ:0.98 (6H, d, J=6.6 Hz), 1.39 (9H, s), 2.14-2.22 (1H, m), 2.34 (3H, s), 2.43 (3H, s), 2.86 (2H, d, J=7.4 Hz), 4.06 (2H, d, J=4.5 Hz), 4.28 (1H, brs), 4.35 (2H, s), 6.97 (2H, d, J=7.9 Hz), 7.23 (2H, d, J=7.7 Hz), 7.60 (2H, d, J=8.1 Hz), 8.17 (2H, d, J=8.1 Hz).

2) 4-({[5-(Aminomethyl)-6-isobutyl-2-methyl-4-(4-methylphenyl)pyridin-3-yl]methyl}sulfonyl)benzoic acid dihydrochloride (279 mg, yield 97%) was obtained as a white powder from 4-({[5-{[(tert-butoxycarbonyl)amino]methyl}-6-isobutyl-2-methyl-4-(4-methylphenyl)pyridin-3-yl]methyl}sulfonyl)benzoic acid (300 mg, 0.530 mmol) according to a method similar to the method of Example 2-3).

¹H-NMR (DMSO-d₆) δ:0.97 (6H, d, J=6.6 Hz), 2.17-2.24 (1H, m), 2.38 (3H, s), 2.76 (3H, brs), 2.95 (2H, brs), 3.70 (2H, brs), 7.05 (2H, d, J=7.9 Hz), 7.23 (2H, d, J=7.9 Hz), 7.62 (2H, d, J=8.3 Hz), 8.07 (2H, d, J=8.3 Hz), 8.24 (3H, brs).

### Example 187

### N-{[5-(aminomethyl)-6-isobutyl-2-methyl-4-(4-methylphenyl)pyridin-3-yl]methyl}methanesulfonamide dihydrochloride

1) To a solution (10 mL) of tert-butyl {[5-(aminomethyl)-2-isobutyl-6-methyl-4-(4-methylphenyl)pyridin-3-yl]methyl}carbamate (200 mg, 0.755 mmol) and triethylamine (0.14 mL, 1.00 mmol) in tetrahydrofuran was added methanesulfonyl chloride (86 mg, 0.875 mmol) and the mixture was stirred at room temperature for 1 hr. The reaction mixture was diluted with ethyl acetate (100 mL) and washed successively with saturated aqueous sodium hydrogen carbonate and saturated brine. The organic layer was dried over anhydrous magnesium sulfate. The solvent was evaporated under reduced pressure and the obtained yellow solid was washed with diisopropyl ether to give tert-butyl [(2-isobutyl-6-methyl-4-(4-methylphenyl)-5-{[(methylsulfonyl)amino]methyl}pyridin-3-yl)methyl]carbamate (210 mg, yield 87%) as a white solid.

¹H-NMR (CDCl₃) δ:0.96 (6H, d, J=6.6 Hz), 1.38 (9H, s), 2.16-2.25 (1H, m), 2.42 (3H, s), 2.61 (3H, s), 2.68 (3H, s), 2.76 (2H, d, J=7.4 Hz), 3.87 (1H, brs), 4.01 (2H, d, J=5.7 Hz), 4.03 (2H, d, J=5.3 Hz), 4.18 (1H, brs), 7.03 (2H, d, J=8.1 Hz), 7.29 (2H, d, J=7.9 Hz).

2) N-{[5-(Aminomethyl)-6-isobutyl-2-methyl-4-(4-methylphenyl)pyridin-3-yl]methyl}methanesulfonamide dihydrochloride (126 mg, yield 64%) was obtained as a white powder from tert-butyl [(2-isobutyl-6-methyl-4-(4-methylphenyl)-5-{[(methylsulfonyl)amino]methyl}pyridin-3-yl)methyl]carbamate (210 mg, 0.441 mmol) according to a method similar to the method of Example 2-3).

¹H-NMR (DMSO-d₆) δ:0.96 (6H, d, J=6.6 Hz), 2.12-2.23 (1H, m), 2.41 (3H, s), 2.71 (3H, s), 2.84 (3H, brs), 3.04 (2H, brs), 3.76 (2H, brs), 3.87 (2H, brs), 7.19 (1H, brs), 7.29 (2H, d, J=7.5 Hz), 7.38 (2H, d, J=7.7 Hz), 8.28 (3H, brs).

### Example 188

### {[4-(2,4-dichlorophenyl)-6-(4-fluorophenyl)-2-isobutylpyridin-3-yl]methyl}amine dihydrochloride

1) (2E)-3-(2,4-Dichlorophenyl)-1-(4-fluorophenyl)prop-2-en-1-one (10.3 g, yield 64%) was obtained as a pale-yellow solid from 4-fluoroacetophenone (6.91 g, 50 mmol) and 2,6-dichlorobenzamide (8.75 g, 59 mmol) according to a method similar to the method of Example 108-1).

¹H-NMR (CDCl₃) δ: 7.16-7.23 (2H, m), 7.31 (1H, dd, J=8.5, 2.1 Hz), 7.42-7.49 (2H, m), 7.68 (2H, d, J=8.5 Hz), 8.07 (3H, m).

2) 4-(2,4-Dichlorophenyl)-6-(4-fluorophenyl)-2-isobutylnicotinonitrile (2.94 g, yield 48%) was obtained as a yellow oil from (2E)-3-(2,4-dichlorophenyl)-1-(4-fluorophenyl)prop-2-en-1-one (4.54 g, 15.4 mmol) according to a method similar to the method of Example 108-2).

¹H-NMR (CDCl₃) δ:1.06 (6H, d, J=6.6 Hz), 2.32-2.45 (1H, m), 3.04 (2H, d, J=7.2 Hz), 7.09-7.24 (3H, m), 7.33 (1H, d, J=8.3 Hz), 7.37-7.44 (1H, m), 7.57 (1H, s), 7.59 (1H, d, J=1.9 Hz), 8.06-8.12 (1H, m).

3) {(4-(2,4-Dichlorophenyl)-6-(4-fluorophenyl)-2-isobutylpyridin-3-yl]methyl}amine (780 mg, yield 68%) was obtained as a pale-yellow oil from 4-(2,4-dichlorophenyl)-6-(4-fluorophenyl)-2-isobutylnicotinonitrile (1.14 g, 2.85 mmol) according to a method similar to the method of Example 23-4). The oil was dissolved in 4N hydrogen chloride 1,4-dioxane solution (20 mL) and the mixture was stirred at room temperature for 30 min. The solvent was evaporated under reduced pressure and the obtained pale-yellow solid was washed with diisopropyl ether to give {[4-(2,4-dichlorophenyl)-6-(4-fluorophenyl)-2-isobutylpyridin-3-yl]methyl}amine dihydrochloride (895 mg, yield 97%) as a pale-yellow powder.

¹H-NMR (DMSO-d₆) δ:0.97 (3H, d, J=6.6 Hz), 1.05 (3H, d, J=6.6 Hz), 2.29-2.38 (1H, m), 2.81-2.99 (2H, m), 3.57-3.64 (1H, m), 4.04-4.16 (1H, m), 7.33 (2H, t, J=8.8 Hz), 7.59-7.67 (2H, m), 7.73 (1H, s), 7.86 (1H, d, J=1.9 Hz), 8.21-8.30 (5H, m).

### Example 189

### methyl 3-[5-(aminomethyl)-6-isobutyl-4-(4-methylphenyl)pyridin-2-yl]benzoate dihydrochloride

1) (2E)-1-(3-Bromophenyl)-3-(4-methylphenyl)prop-2-en-1-one (7.09 g, yield 47%) was obtained as a pale-yellow powder from 3-bromoacetophenone (9.95 g, 50 mmol) according to a method similar to the method of Example 108-1).

2) 6-(3-Bromophenyl)-2-isobutyl-4-(4-methylphenyl)nicotinonitrile (2.20 g, yield 32%) was obtained as a pale-yellow solid from (2E)-1-(3-bromophenyl)-3-(4-methylphenyl)prop-2-en-1-one (5.03 g, 16.7 mmol) according to a method similar to the method of Example 108-2).

¹H-NMR (CDCl₃) δ:1.06 (6H, d, J=6.6 Hz), 2.35-2.42 (1H, m), 2.45 (3H, s), 3.06 (2H, d, J=7.4 Hz), 7.09-7.16 (3H, m), 7.30-7.40 (4H, m), 7.53-7.55 (1H, m), 7.64 (1H, s).

3) 6-(3-Bromophenyl)-2-isobutyl-4-(4-methylphenyl)nicotinonitrile (2.20 g, 5.40 mmol), triethylamine (0.70 mL, 10.0 mmol) and [1,1′-bis(diphenylphosphino)ferrocene]palladium(II) dichloride (410 mg, 0.500 mmol) were dissolved in a mixed solvent of methanol (10 mL)-N,N-dimethylformamide (30 mL) and the mixture was stirred under a carbon monoxide atmosphere for 15 hrs. The reaction mixture was diluted with ethyl acetate (100 mL) and the mixture was washed with saturated brine. The organic layer was dried over anhydrous magnesium sulfate and the solvent was evaporated under reduced pressure. The obtained residue was purified by silica gel column chromatography to give methyl 3-[5-cyano-6-isobutyl-4-(4-methylphenyl)pyridin-2-yl]benzoate (1.39 g, yield 72%) as a colorless oil. Methyl 3-[5-(aminomethyl)-6-isobutyl-4-(4-methylphenyl)pyridin-2-yl]benzoate (780 mg, yield 58%) was obtained as a colorless oil from methyl 3-[5-cyano-6-isobutyl-4-(4-methylphenyl)pyridin-2-yl]benzoate (1.30 g, 3.38 mmol) according to a method similar to the method of Example 1-4).

¹H-NMR (CDCl₃) δ:1.05 (6H, d, J=6.6 Hz), 2.37-2.48 (4H, m), 2.90 (2H, d, J=7.2 Hz), 3.84 (2H, s), 3.94 (3H, s), 7.27-7.33 (4H, m), 7.49 (1H, s), 7.54 (1H, t, J=7.9 Hz), 8.04-8.07 (1H, m), 8.32 (1H, m), 8.61-8.62 (1H, m).

4) Methyl 3-[5-{[(tert-butoxycarbonyl)amino]methyl}-6-isobutyl-4-(4-methylphenyl)pyridin-2-yl]benzoate (730 mg, yield 76%) was obtained as a white powder from methyl 3-[5-(aminomethyl)-6-isobutyl-4-(4-methylphenyl)pyridin-2-yl]benzoate (0.76 g, 1.96 mmol) according to a method similar to the method of Example 2-1).

¹H-NMR (CDCl₃) δ:1.04 (6H, d, J=6.6 Hz), 1,43 (9H, s), 2.37-2.46 (4H, m), 2.87 (2H, d, J=7.2 Hz), 3.94 (3H, s), 4.29-4.35 (2H, m), 4.38 (1H, brs), 7.23 (2H, d, J=8.3 Hz), 7.28 (2H, d, J=8.1 Hz), 7.50 (1H, s), 7.54 (1H, t, J=7.8 Hz), 8.05-8.08 (1H, m), 8.30-8.34 (1H, m), 8.62-8.63 (1H, m).

5) Methyl 3-[5-(aminomethyl)-6-isobutyl-4-(4-methylphenyl)pyridin-2-yl]benzoate dihydrochloride (188 mg, yield 99%) was obtained as a white powder from methyl 3-[5-{[(tert-butoxycarbonyl)amino]methyl}-6-isobutyl-4-(4-methylphenyl)pyridin-2-yl]benzoate (200 mg, 0.409 mmol) according to a method similar to the method of Example 2-3).

¹H-NMR (DMSO-d₆) δ:1.04 (6H, d, J=6.4 Hz), 2.33-2.44 (4H, m), 2.93 (2H, d, J=7.0 Hz), 3.90 (3H, s), 4.01 (2H, d, J=5.5 Hz), 7.36 (2H, d, J=8.1 Hz), 7.41 (2H, d, J=8.3 Hz), 7.66 (1H, t, J=7.8 Hz), 7.76 (1H, s), 8.01-8.08 (1H, m), 8.40 (3H, brs), 8.42-8.47 (1H, m), 8.71-8.75 (1H, m).

### Example 190

### 3-[5-(aminomethyl)-6-isobutyl-4-(4-methylphenyl)pyridin-2-yl]benzoic acid dihydrochloride

1) 3-[5-{[(tert-Butoxycarbonyl)amino]methyl}-6-isobutyl-4-(4-methylphenyl)pyridin-2-yl]benzoic acid (500 mg, yield 98%) was obtained as a white solid from methyl 3-[5-{[(tert-butoxycarbonyl)amino]methyl}-6-isobutyl-4-(4-methylphenyl)pyridin-2-yl]benzoate (530 mg, 1.08 mmol) according to a method similar to the method of Example 9-1).

¹H-NMR (CDCl₃) δ:1.05 (6H, d, J=6.6 Hz), 1,43 (9H, s), 2.35-2.47 (4H, m), 2.92 (2H, brs), 4.31-4.37 (2H, m), 4.42 (1H, brs), 7.22-7.30 (4H, m), 7.52 (1H, s), 7.58 (1H, t, J=7.5 Hz), 8.12 (1H, d, J=7.9 Hz), 8.36 (1H, d, J=7.4 Hz), 8.67 (1H, s).

2) 3-[5-(Aminomethyl)-6-isobutyl-4-(4-methylphenyl)pyridin-2-yl]benzoic acid dihydrochloride (188 mg, yield 99%) was obtained as a white powder from 3-[5-{[(tert-butoxycarbonyl)amino]methyl}-6-isobutyl-4-(4-methylphenyl)pyridin-2-yl]benzoic acid (200 mg, 0.421 mmol) according to a method similar to the method of Example 2-3).

¹H-NMR (DMSO-d₆) δ:1.03 (6H, d, J=7.4 Hz), 2.32-2.43 (4H, m), 2.92 (2H, d, J=7.0 Hz), 4.02 (2H, d, J=5.3 Hz), 7.36 (2H, d, J=8.1 Hz), 7.41 (2H, d, J=8.3 Hz), 7.63 (1H, t, J=7.8 Hz), 7.74 (1H, s), 8.01-8.04 (1H, m), 8.35 (3H, brs), 8.37-8.41 (1H, m), 8.71-8.72 (1H, m).

### Example 191

### 3-[5-(aminomethyl)-6-isobutyl-4-(4-methylphenyl)pyridin-2-yl]benzamide dihydrochloride

1) tert-Butyl {[6-[3-(aminocarbonyl)phenyl]-2-isobutyl-4-(4-methylphenyl)pyridin-3-yl]methyl}carbamate (160 mg, yield 53%) was obtained as a white solid from 3-[5-{[(tert-butoxycarbonyl)amino]methyl}-6-isobutyl-4-(4-methylphenyl)pyridin-2-yl]benzoic acid (300 mg, 0.632 mmol) according to a method similar to the method of Example 3-1).

¹H-NMR (CDCl₃) δ:1.04 (6H, d, J=6.6 Hz), 1,43 (9H, s), 2.34-2.48 (4H, m), 2.87 (2H, d, J=7.2 Hz), 4.32 (2H, d, J=4.7 Hz), 4.39 (1H, brs), 7.22 (2H, d, J=8.1 Hz), 7.25-7.29 (2H, m), 7.50 (1H, s), 7.55 (1H, t, J=7.8 Hz), 7.83-7.87 (1H, m), 8.21-8.25 (1H, m), 8.45-8.46 (1H, m).

2) 3-[5-(Aminomethyl)-6-isobutyl-4-(4-methylphenyl)pyridin-2-yl]benzamide dihydrochloride (127 mg, yield 84%) was obtained as a white powder from tert-butyl {[6-[3-(aminocarbonyl)phenyl]-2-isobutyl-4-(4-methylphenyl)pyridin-3-yl]methyl}carbamate (160 mg, 0.338 mmol) according to a method similar to the method of Example 2-3).

¹H-NMR (DMSO-d₆) δ:1.03 (6H, d, J=6.6 Hz), 2.34-2.44 (4H, m), 2.93 (2H, d, J=7.0 Hz), 4.01 (2H, d, J=5.5 Hz), 7.37 (2H, d, J=8.1 Hz), 7.42 (2H, d, J=8.1 Hz), 7.47 (1H, brs), 7.60 (1H, t, J=7.8 Hz), 7.81 (1H, s), 7.96 (1H, d, J=7.7 Hz), 8.14 (1H, brs), 8.33-8.44 (4H, m), 8.58 (1H, s).

### Example 192

### methyl 2-[5-(aminomethyl)-6-isobutyl-4-(4-methylphenyl)pyridin-2-yl]benzoate dihydrochloride

1) (2E)-1-(2-Bromophenyl)-3-(4-methylphenyl)prop-2-en-1-one (8.86 g, yield 44%) was obtained as a pale-yellow powder from 2-bromoacetophenone (9.95 g, 50 mmol) according to a method similar to the method of Example 108-1).

2) 6-(2-Bromophenyl)-2-isobutyl-4-(4-methylphenyl)nicotinonitrile (3.58 g, yield 53%) was obtained as a pale-yellow solid from (2E)-1-(2-bromophenyl)-3-(4-methylphenyl)prop-2-en-1-one (5.03 g, 16.7 mmol) according to a method similar to the method of Example 108-2).

¹H-NMR (CDCl₃) δ:1.06 (6H, d, J=6.6 Hz), 2.34-2.44 (4H, m), 3.07 (2H, d, J=7.4 Hz), 7.27-7.30 (1H, m), 7.32-7.36 (2H, m), 7.41-7.47 (1H, m), 7.53-7.60 (3H, m), 7.71 (1H, m).

3) Methyl 2-[5-cyano-6-isobutyl-4-(4-methylphenyl)pyridin-2-yl]benzoate (1.80 g, yield 76%) was obtained as a colorless oil from 6-(2-bromophenyl)-2-isobutyl-4-(4-methylphenyl)nicotinonitrile (2.50 g, 6.14 mmol) according to a method similar to the method of Example 189-3). That is, 6-(2-bromophenyl)-2-isobutyl-4-(4-methylphenyl)nicotinonitrile, triethylamine (1.7 mL, 12.2 mmol) and [1,1′-bis(diphenylphosphino)ferrocene]palladium(II) dichloride (501 mg, 0.614 mmol) were dissolved in methanol (7.5 mL)-N,N-dimethylformamide (15 mL) and the mixture was stirred under a carbon monoxide atmosphere for 13 hrs. The reaction mixture was diluted with ethyl acetate (100 mL) and the mixture was washed with saturated brine. The organic layer was dried over anhydrous magnesium sulfate and the solvent was evaporated under reduced pressure. The obtained residue was purified by silica gel column chromatography to give methyl 2-[5-cyano-6-isobutyl-4-(4-methylphenyl)pyridin-2-yl]benzoate.

¹H-NMR (CDCl₃) δ:1.03 (6H, d, J=6.8 Hz), 2.26-2.37 (1H, m), 2.44 (3H, s), 3.01 (2H, d, J=7.4 Hz), 3.74 (3H, s), 7.08-7.14 (1H, m), 7.34 (2H, d, J=7.9 Hz), 7.42 (1H, s), 7.48-7.61 (4H, m), 7.83-7.88 (1H, m).

4) Methyl 2-[5-(aminomethyl)-6-isobutyl-4-(4-methylphenyl)pyridin-2-yl]benzoate was obtained as a crude product from methyl 2-[5-cyano-6-isobutyl-4-(4-methylphenyl)pyridin-2-yl]benzoate (1.80 g, 4.68 mmol) according to a method similar to the method of Example 1-4). Methyl 2-[5-{[(tert-butoxycarbonyl)amino]methyl}-6-isobutyl-4-(4-methylphenyl)pyridin-2-yl]benzoate (1.70 g, yield 74%) was obtained as a colorless oil from the crude product according to a method similar to the method of Example 2-1).

¹H-NMR (CDCl₃) δ:0.99 (6H, d, J=6.6 Hz), 1.43 (9H, s), 2.26-2.37 (1H, m), 2.41 (3H, s), 2.80 (2H, d, J=7.4 Hz), 3.75 (3H, s), 4.32 (2H, d, J=4.9 Hz), 4.42 (1H, brs), 7.21-7.27 (5H, m), 7.41-7.46 (1H, m), 7.52-7.58 (2H, m), 7.76 (1H, dd, J=7.4, 1.1 Hz).

5) Methyl 2-[5-(aminomethyl)-6-isobutyl-4-(4-methylphenyl)pyridin-2-yl]benzoate dihydrochloride (345 mg, yield 95%) was obtained as a pale-pink powder from methyl 2-[5-{[(tert-butoxycarbonyl)amino]methyl}-6-isobutyl-4-(4-methylphenyl)pyridin-2-yl]benzoate (383 mg, 0.786 mmol) according to a method similar to the method of Example 2-3).

¹H-NMR (DMSO-d₆) δ:0.97 (6H, d, J=6.6 Hz), 2.18-2.32 (1H, m), 2.41 (3H, s), 2.89 (2H, d, J=6.6 Hz), 3.69 (3H, s), 3.99-4.09 (2H, m), 7.36 (2H, d, J=8.1 Hz), 7.43 (2H, d, J=8.1 Hz), 7.49 (1H, s), 7.57-7.70 (2H, m), 7.76 (2H, d, J=7.5 Hz), 8.51 (3H, brs).

### Example 193

### 2-[5-(aminomethyl)-6-isobutyl-4-(4-methylphenyl)pyridin-2-yl]benzoic acid dihydrochloride

1) 2-[5-{[(tert-Butoxycarbonyl)amino]methyl}-6-isobutyl-4-(4-methylphenyl)pyridin-2-yl]benzoic acid (0.85 g, yield 67%) was obtained as a colorless oil from methyl 2-[5-{[(tert-butoxycarbonyl)amino]methyl}-6-isobutyl-4-(4-methylphenyl)pyridin-2-yl]benzoate (1.31 g, 2.69 mmol) according to a method similar to the method of Example 9-1).

¹H-NMR (CDCl₃) δ:1.02 (6H, d, J=6.6 Hz), 1.42 (9H, s), 2.21-2.33 (1H, m), 2.44 (3H, s), 2.93 (2H, d, J=7.4 Hz), 4.39 (2H, brs), 7.22 (2H, d, J=8.1 Hz), 7.31 (2H, d, J=7.9 Hz), 7.48 (1H, s), 7.54-7.66 (3H, m), 8.31 (1H, m).

2) 2-[5-(Aminomethyl)-6-isobutyl-4-(4-methylphenyl)pyridin-2-yl]benzoic acid dihydrochloride (329 mg, yield 81%) was obtained as a white powder from 2-[5-{[(tert-butoxycarbonyl)amino]methyl}-6-isobutyl-4-(4-methylphenyl)pyridin-2-yl]benzoic acid (429 mg, 0.904 mmol) according to a method similar to the method of Example 2-3).

¹H-NMR (DMSO-d₆) δ:0.99 (6H, d, J=6.6 Hz), 2.27-2.36 (1H, m), 2.41 (3H, s), 2.90 (2H, d, J=6.6 Hz), 4.04 (2H, d, J=5.1 Hz), 7.36 (2H, d, J=8.3 Hz), 7.40-7.49 (3H, m), 7.54-7.70 (3H, m), 7.76-7.84 (1H, m), 8.44 (3H, brs).

### Example 194

### 2-[5-(aminomethyl)-6-isobutyl-4-(4-methylphenyl)pyridin-2-yl]benzamide dihydrochloride

1) tert-Butyl {[6-[2-(aminocarbonyl)phenyl]-2-isobutyl-4-(4-methylphenyl)pyridin-3-yl]methyl}carbamate (290 mg, yield 69%) was obtained as a colorless oil from 2-[5-{[(tert-butoxycarbonyl)amino]methyl}-6-isobutyl-4-(4-methylphenyl)pyridin-2-yl]benzoic acid (421 mg, 0.887 mmol) according to a method similar to the method of Example 3-1).

¹H-NMR (CDCl₃) δ:1.01 (6H, d, J=6.6 Hz), 1.43 (9H, s), 2.30-2.37 (1H, m), 2.41 (3H, s), 2.83 (2H, d, J=7.4 Hz), 4.34 (2H, d, J=4.7 Hz), 4.42 (1H, brs), 5.54 (1H, brs), 6.42 (1H, brs), 7.20 (2H, d, J=8.3 Hz), 7.24-7.25 (3H, m), 7.42-7.53 (3H, m), 7.70-7.75 (1H, m).

2) 2-[5-(Aminomethyl)-6-isobutyl-4-(4-methylphenyl)pyridin-2-yl]benzamide dihydrochloride (254 mg, yield 93%) was obtained as a yellow powder from tert-butyl {[6-[2-(aminocarbonyl)phenyl]-2-isobutyl-4-(4-methylphenyl)pyridin-3-yl]methyl}carbamate (290 mg, 0.612 mmol) according to a method similar to the method of Example 2-3).

¹H-NMR (DMSO-d₆) δ:1.01 (6H, d, J=6.6 Hz), 2.27-2.37 (1H, m), 2.40 (3H, s), 2.90-2.99 (2H, m), 4.04 (2H, m), 7.36 (2H, d, J=8.1 Hz), 7.41 (2H, d, J=8.3 Hz), 7.50 (1H, s), 7.56-7.71 (4H, m), 7.92-8.01 (1H, m), 8.61 (3H, brs).

### Example 195

### 5-(aminomethyl)-N,N-dicyclohexyl-6-isobutyl-2-methyl-4-(4-methylphenyl)nicotinamide dihydrochloride

1) 5-Cyano-6-isobutyl-2-methyl-4-(4-methylphenyl)nicotinic acid (2.16 g, yield 85%) was obtained as a white powder from tert-butyl 5-cyano-6-isobutyl-2-methyl-4-(4-methylphenyl)nicotinate (3.00 g, 8.23 mmol) according to a method similar to the method of Example 24-1).

¹H-NMR (CDCl₃) δ:1.00 (6H, d, J=6.6 Hz), 2.17-2.32 (1H, m), 2.42 (3H, s), 2.67 (3H, s), 2.95 (2H, d, J=7.4 Hz), 7.27-7.34 (4H, m).

2) To a solution of 5-cyano-6-isobutyl-2-methyl-4-(4-methylphenyl)nicotinic acid (2.00 g, 6.49 mmol) in dichloromethane were added oxalyl chloride (0.68 mL, 7.78 mmol) and N,N-dimethylformamide (0.05 mL) and the mixture was stirred at room temperature for 30 min. The solvent was evaporated under reduced pressure and the residue was dissolved in tetrahydrofuran. Subsequently, triethylamine (1.8 mL, 13.0 mmol) and dicyclohexylamine (1.55 mL, 7.78 mmol) were added and the mixture was stirred at room temperature for 30 min. The reaction mixture was diluted with ethyl acetate (100 mL) and washed with saturated brine. The organic layer was dried over anhydrous magnesium sulfate. The solvent was evaporated under reduced pressure and the obtained residue was purified by silica gel column chromatography to give 5-cyano-N,N-dicyclohexyl-6-isobutyl-2-methyl-4-(4-methylphenyl)nicotinamide (0.35 g, yield 11%) as a colorless oil.

¹H-NMR (CDCl₃) δ:0.79-0.96 (4H, m), 1.01 (6H, dd, J=11.1, 6.6 Hz), 1.07-1.34 (4H, m), 1.40-1.53 (5H, m), 1.58-1.68 (4H, m), 1.72-1.84 (3H, m), 2.22-2.31 (1H, m), 2.40 (3H, s), 2.59 (3H, s), 2.69-2.79 (2H, m), 2.87-3.04 (2H, m), 7.25 (2H, d, J=8.5 Hz), 7.46 (2H, d, J=8.1 Hz).

3) 5-(Aminomethyl)-N,N-dicyclohexyl-6-isobutyl-2-methyl-4-(4-methylphenyl)nicotinamide dihydrochloride (0.20 g, yield 49%) was obtained as a yellow powder from 5-cyano-N,N-dicyclohexyl-6-isobutyl-2-methyl-4-(4-methylphenyl)nicotinamide (0.35 g, 0.742 mmol) according to a method similar to the method of Example 108-3).

¹H-NMR (DMSO-d₆) δ:0.73-0.88 (2H, m), 0.90-1.15 (12H, m), 1.24-1.75 (10H, m), 2.13-2.27 (3H, m), 2.36 (3H, s), 2.78-2.86 (2H, m), 2.88-2.95 (2H, m), 3.68-3.81 (1H, m), 3.96-4.09 (1H, m), 7.26-7.37 (4H, m).

### Example 196

### methyl 1-{[5-(aminomethyl)-6-isobutyl-2-methyl-4-(4-methylphenyl)pyridin-3-yl]carbonyl}piperidine-4-carboxylate dihydrochloride

1) Methyl 1-{[5-cyano-6-isobutyl-2-methyl-4-(4-methylphenyl)pyridin-3-yl]carbonyl}piperidine-4-carboxylate (3.20 g, yield 91%) was obtained as a colorless oil from 5-cyano-6-isobutyl-2-methyl-4-(4-methylphenyl)nicotinic acid (2.50 g, 8.1 mmol) and methyl isonipecotate (1.3 mL, 9.73 mmol) according to a method similar to the method of Example 195-2).

¹H-NMR (CDCl₃) δ:1.01 (6H, dd, J=12.1, 6.6 Hz), 1.42-1.85 (4H, m), 2.19-2.37 (3H, m), 2.40 (3H, s), 2.55-2.60 (3H, m), 2.61-3.20 (5H, m), 3.63-3.66 (3H, m), 4.23-4.45 (1H, m), 7.25-7.42 (4H, m).

2) Methyl 1-{[5-(aminomethyl)-6-isobutyl-2-methyl-4-(4-methylphenyl)pyridin-3-yl]carbonyl}piperidine-4-carboxylate dihydrochloride (3.27 g, yield 87%) was obtained as a white powder from methyl 1-{[5-cyano-6-isobutyl-2-methyl-4-(4-methylphenyl)pyridin-3-yl]carbonyl}piperidine-4-carboxylate (3.20 g, 7.38 mmol) according to a method similar to the method of Example 108-3).

¹H-NMR (DMSO-d₆) δ:0.67-0.90 (1H, m), 0.98 (6H, t, J=5.9 Hz), 1.25-1.76 (3H, m), 2.16-2.28 (1H, m), 2.36-2.37 (3H, m), 2.63-2.76 (1H, m), 2.90-3.03 (2H, m), 3.17-3.34 (1H, m), 3.57 (3H, s), 3.58-3.60 (2H, m), 3.68-3.97 (2H, m), 4.05-4.10 (1H, m), 7.11-7.36 (4H, m), 8.34 (3H, brs).

### Example 197

### 5-(aminomethyl)-6-isobutyl-2-methyl-4-(4-methylphenyl)nicotinic acid tert-butylamine salt

5-(Aminomethyl)-6-isobutyl-2-methyl-4-(4-methylphenyl)nicotinic acid (0.10 g, 0.320 mmol) was dissolved in a mixed solvent of water (1.5 mL)-acetonitrile (1.5 mL) with heating under reflux for 10 min. tert-Butylamine (23.4 mg, 0.320 mmol) was added to the obtained solution and the mixture was stirred at the same temperature for 10 min. Acetonitrile (20 mL) was added, and the mixture was allowed to cool to room temperature and stirred at 0° C. for 30 min. The precipitated solid was collected by filtration and washed with acetonitrile (10 mL) to give 5-(aminomethyl)-6-isobutyl-2-methyl-4-(4-methylphenyl)nicotinic acid tert-butylamine salt (78.4 mg, yield 63%) as a white powder.

¹H-NMR (DMSO-d₆) δ:0.91 (6H, d, J=6.6 Hz), 1.12 (9H, s), 2.06-2.25 (1H, m), 2.31 (3H, s), 2.34 (3H, s), 2.66 (2H, d, J=7.0 Hz), 3.31 (2H, brs), 3.37 (2H, s), 7.10 (2H, d, J=8.1 Hz), 7.16 (2H, d, J=8.1 Hz).

### Example 198

### ({2-isobutyl-6-methyl-4-(4-methylphenyl)-5-[(methylthio)methyl]pyridin-3-yl}methyl)amine dihydrochloride

1) To a solution of [5-{[(tert-butoxycarbonyl)amino]methyl}-6-isobutyl-2-methyl-4-(4-methylphenyl)pyridin-3-yl]methyl methanesulfonate (476 mg, 1 mmol) in tetrahydrofuran (5 mL) was added 15% aqueous sodium methanethiolate solution (3 mL) and the mixture was stirred at 50° C. for 2 hrs. Water was added to the reaction mixture and the mixture was extracted with ethyl acetate. The organic layer was washed with saturated brine and dried over anhydrous magnesium sulfate. The solvent was evaporated under reduced pressure and the obtained residue was purified by silica gel column chromatography to give tert-butyl ({2-isobutyl-6-methyl-4-(4-methylphenyl)-5-[(methylthio)methyl]pyridin-3-yl}methyl)carbamate (312 mg, yield 72%) as a white powder.

¹H-NMR (CDCl₃) δ:0.96 (6H, d, J=6.6 Hz), 1.38 (9H, s), 1.94 (3H, s), 2.12-2.23 (1H, m), 2.42 (3H, s), 2.67 (3H, s), 2.75 (2H, d, J=6.9 Hz), 3.39 (2H, s), 4.02 (2H, d, J=5.7 Hz), 4.19 (1H, brs), 7.04 (2H, d, J=8.1 Hz), 7.24 (2H, d, J=8.1 Hz).

2) ({2-Isobutyl-6-methyl-4-(4-methylphenyl)-5-[(methylthio)methyl]pyridin-3-yl}methyl)amine dihydrochloride (36 mg, yield 96%) was obtained as a white powder from tert-butyl ({2-isobutyl-6-methyl-4-(4-methylphenyl)-5-[(methylthio)methyl]pyridin-3-yl}methyl)carbamate according to a method similar to the method of Example 2-3).

¹H-NMR (DMSO-d₆) δ:0.97 (6H, d, J=6.6 Hz), 1.93 (3H, s), 2.12-2.19(1H, m), 2.42 (3H, s), 2.89 (3H, s), 3.08 (2H, brs), 3.48 (2H, s), 3.75 (2H, s), 7.28 (2H, d, J=7.8 Hz), 7.39 (2H, d, J=7.8 Hz), 8.36 (3H, brs).

### Example 199

### ({2-isobutyl-6-methyl-4-(4-methylphenyl)-5-[(methylsulfonyl)methyl]pyridin-3-yl}methyl)amine dihydrochloride

1) To a solution of tert-butyl ({2-isobutyl-6-methyl-4-(4-methylphenyl)-5-[(methylthio)methyl]pyridin-3-yl}methyl)carbamate (200 mg, 0.46 mmol) in methanol-water (10:1, 5 mL) was added Oxone (trademark, 310 mg) and then sulfuric acid (50 μL) was added. The mixture was stirred at room temperature for 6 hrs. Aqueous saturated sodium hydrogen carbonate was added to the reaction mixture and the mixture was extracted with ethyl acetate. The organic layer was washed with saturated brine and dried over anhydrous magnesium sulfate. The solvent was evaporated under reduced pressure and the obtained residue was purified by silica gel column chromatography to give tert-butyl ({2-isobutyl-6-methyl-4-(4-methylphenyl)-5-((methylsulfonyl)methyl]pyridin-3-yl}methyl)carbamate (128 mg, yield 60%) as a white powder.

¹H-NMR (CDCl₃) δ:0.97 (6H, d, J=6.6 Hz), 1.38 (9H, s), 2.19-2.28 (1H, m), 2.41 (3H, s), 2.61 (3H, s), 2.74 (3H, s), 2.75 (2H, d, J=7.2 Hz), 4.25 (2H, d, J=5.1 Hz), 4.24 (1H, brs), 4.26 (2H, s), 7.71 (2H, d, J=7.8 Hz), 7.26 (2H, d, J=8.1 Hz).

2) ({2-Isobutyl-6-methyl-4-(4-methylphenyl)-5-[(methylsulfonyl)methyl]pyridin-3-yl}methyl)amine dihydrochloride (36 mg, yield 96%) was obtained as a white powder from tert-butyl ({2-isobutyl-6-methyl-4-(4-methylphenyl)-5-[(methylsulfonyl)methyl]pyridin-3-yl)methyl)carbamate according to a method similar to the method of Example 2-3).

¹H-NMR (DMSO-d₆) δ:0.97 (6H, d, J=6.6 Hz), 2.17-2.24 (1H, m), 2.40 (3H, s), 2.81 (3H, s), 2.87 (3H, s), 2.89 (2H, brs), 3.68 (2H, brs), 4.40 (2H, s), 7.24 (2H, d, J=8.1 Hz), 7.35 (2H, d, J=7.8 Hz), 8.20 (3H, brs).

### Example 200

### ({[5-(aminomethyl)-6-isobutyl-2-methyl-4-(4-methylphenyl)pyridin-3-yl]methyl}thio)acetic acid dihydrochloride

1) To a solution of [5-{[(tert-butoxycarbonyl)amino]methyl}-6-isobutyl-2-methyl-4-(4-methylphenyl)pyridin-3-yl]methyl methanesulfonate (952 mg, 2 mmol) in N,N-dimethylformamide (5 mL) was added potassium carbonate (415 mg, 3 mmol) and then ethyl mercaptoacetate (240 μL, 2.2 mmol) was added. The mixture was stirred at 50° C. for 1 hr. Water was added to the reaction mixture and the mixture was extracted with ethyl acetate. The organic layer was washed with saturated brine and dried over anhydrous magnesium sulfate. The solvent was evaporated under reduced pressure and the obtained residue was dissolved in ethanol (5 mL). 1N Aqueous sodium hydroxide solution (5 mL) was added and the mixture was stirred at room temperature for 2 hrs. 1N Hydrochloric acid (5 mL) was added to the reaction mixture and the mixture was extracted with ethyl acetate. The organic layer was washed with saturated brine and dried over anhydrous magnesium sulfate. The solvent was evaporated under reduced pressure and the obtained residue was purified by silica gel column chromatography to give ({[5-{[(tert-butoxycarbonyl)amino]methyl}-6-isobutyl-2-methyl-4-(4-methylphenyl)pyridin-3-yl]methyl}thio)acetic acid (265 mg, yield 27%) as a white powder.

¹H-NMR (DMSO-d₆) δ:0.91 (6H, d, J=6.6 Hz), 1.34 (9H, s), 2.13-2.27 (1H, m), 2.37 (3H, s), 2.55 (2H, d, J=6.0 Hz), 2.58 (3H, s), 3.09 (2H, s), 3.50 (2H, s), 3.74 (2H, d, J=4.2 Hz), 6.81 (1H, brs), 7.18 (2H, d, J=8.1 Hz), 7.24 (2H, d, J=8.1 Hz), 12.49 (1H, brs).

2) ({[5-(Aminomethyl)-6-isobutyl-2-methyl-4-(4-methylphenyl)pyridin-3-yl]methyl}thio)acetic acid dihydrochloride (106 mg, yield 96%) was obtained as a white powder from ({[5-{[(tert-butoxycarbonyl)amino]methyl}-6-isobutyl-2-methyl-4-(4-methylphenyl)pyridin-3-yl]methyl}thio)acetic acid according to a method similar to the method of Example 2-3).

¹H-NMR (DMSO-d₆) δ:0.96 (6H, d, J=6.6 Hz), 2.14-2.25 (1H, m), 2.42 (3H, s), 2.85 (3H, brs), 3.01 (2H, s), 3.20 (2H, s), 3.59 (2H, s), 3.70 (2H, s), 7.26 (2H, d, J=8.1 Hz), 7.37 (2H, d, J =8.1 Hz), 8.23 (3H, brs).

### Example 201

### ({[5-(aminomethyl)-6-isobutyl-2-methyl-4-(4-methylphenyl)pyridin-3-yl]methyl}sulfonyl)acetic acid dihydrochloride

1) To a solution of ({[5-{[(tert-butoxycarbonyl)amino]methyl}-6-isobutyl-2-methyl-4-(4-methylphenyl)pyridin-3-yl]methyl}thio)acetic acid (260 mg, 0.55 mmol) in methanol-water (10:1, 5 mL) was added Oxone (trademark, (508 mg) and then sulfuric acid (50 μL) was added. The mixture was stirred at room temperature for 6 hrs. Aqueous saturated sodium hydrogen carbonate was added to the reaction mixture and the mixture was extracted with ethyl acetate. The organic layer was washed with saturated brine and dried over anhydrous magnesium sulfate. The solvent was evaporated under reduced pressure and the obtained residue was purified by silica gel column chromatography to give an oil. ({[5-(Aminomethyl)-6-isobutyl-2-methyl-4-(4-methylphenyl)pyridin-3-yl]methyl}sulfonyl)acetic acid dihydrochloride (104 mg, yield 68%) was obtained as a white powder from the obtained oil according to a method similar to the method of Example 2-3).

¹H-NMR (DMSO-d₆) δ:0.95 (6H, d, J=6.6 Hz), 2.21-2.28 (1H, m), 2.39 (3H, s), 2.65 (3H, s), 2.74 (2H, s), 3.61(2H, s), 4.13 (2H, s), 4.55 (2H, s), 7.18 (2H, d, J=8.1 Hz), 7.29 (2H, d, J=7.8 Hz), 8.01 (3H, brs).

### Example 202

### {[2-isobutyl-6-methyl-4-(4-methylphenyl)-5-(1H-tetrazol-5-ylmethyl)pyridin-3-yl]methyl}amine dihydrochloride

1) To a solution of tert-butyl {[5-(cyanomethyl)-2-isobutyl-6-methyl-4-(4-methylphenyl)pyridin-3-yl]methyl}carbamate (300 mg, 0.74 mmol) in toluene (5 mL) were added dibutyltin oxide (37 mg, 0.15 mmol) and trimethylsilyl azide (292 μL, 2.2 mmol) and the mixture was stirred at 80° C. for 3 days. Water was added to the reaction mixture, and the mixture was extracted with ethyl acetate. The organic layer was washed with saturated brine and dried over anhydrous magnesium sulfate. The solvent was evaporated under reduced pressure and the obtained residue was purified by silica gel column chromatography to give tert-butyl {[2-isobutyl-6-methyl-4-(4-methylphenyl)-5-(1H-tetrazol-5-ylmethyl)pyridin-3-yl]methyl}carbamate (229 mg, yield 69%) as a white powder.

¹H-NMR (CDCl₃) δ:0.90 (6H, d, J=6.6 Hz), 1.36 (9H, s), 2.08-2.11 (1H, m), 2.35 (3H, s), 2.42 (3H, s), 2.83 (2H, s), 4.03(2H, s), 4.09 (2H, brs), 4.79 (1H, brs), 7.01 (2H, d, J=8.1 Hz), 7.18 (2H, d, J=7.8 Hz).

2) {[2-Isobutyl-6-methyl-4-(4-methylphenyl)-5-(1H-tetrazol-5-ylmethyl)pyridin-3-yl]methyl}amine dihydrochloride (181 mg, yield 87%) was obtained as a white powder from tert-butyl {[2-isobutyl-6-methyl-4-(4-methylphenyl)-5-(1H-tetrazol-5-ylmethyl)pyridin-3-yl]methyl}carbamate according to a method similar to the method of Example 2-3).

¹H-NMR (DMSO-d₆) δ:1.00 (6H, d, J=6.6 Hz), 2.15-2.23 (1H, m), 2.36 (3H, s), 2.74 (3H, s), 3.14 (2H, s), 3.78 (2H, s), 4.04 (2H, s), 7.06 (2H, d, J=8.1 Hz), 7.28 (2H, d, J=8.1 Hz), 8.35 (3H, brs).

### Example 203

### 3-{[5-(aminomethyl)-6-isobutyl-2-methyl-4-(4-methylphenyl)pyridin-3-yl]methyl}-1,2,4-oxadiazol-5(4H)-one dihydrochloride

1) To a solution of tert-butyl {[5-(cyanomethyl)-2-isobutyl-6-methyl-4-(4-methylphenyl)pyridin-3-yl]methyl}carbamate (400 mg, 1.0 mmol) in ethanol (5 mL) were added sodium carbonate (420 mg, 4.0 mmol) and hydroxy ammonium chloride (210 mg, 3.0 mmol) and the mixture was stirre at 80° C. for 3 days. Water was added to the reaction mixture and the mixture was extracted with ethyl acetate. The organic layer was washed with saturated brine and dried over anhydrous magnesium sulfate. The solvent was evaporated under reduced pressure and the obtained residue was dissolved in tetrahydrofuran (5 mL). N,N′-Carbonyldiimidazole (350 mg, 2.5 mmol) was added and the mixture was stirred at 80° C. for 4 hrs. The reaction mixture was concentrated and the obtained residue was purified by silica gel column chromatography to give tert-butyl ({2-isobutyl-6-methyl-4-(4-methylphenyl)-5-[(5-oxo-4,5-dihydro-1,2,4-oxadiazol-3-yl)methyl]pyridin-3-yl}methyl)carbamate (120 mg, yield 26%) as a white powder.

¹H-NMR (CDCl₃) δ:0.95 (6H, d, J=6.6 Hz), 1.38 (9H, s), 2.06-2.22 (1H, m), 2.40 (3H, s), 2.51 (3H, s), 2.73 (2H, d, J=7.2 Hz), 3.62(2H, s), 4.02 (2H, d, J=4.5 Hz), 4.45 (1H, brs), 7.02 (2H, d, J=8.1 Hz), 7.26 (2H, d, J=7.8 Hz).

2) 3-{[5-(Aminomethyl)-6-isobutyl-2-methyl-4-(4-methylphenyl)pyridin-3-yl]methyl}-1,2,4-oxadiazol-5(4H)-one dihydrochloride (181 mg, yield 87%) was obtained as a white powder from tert-butyl ({2-isobutyl-6-methyl-4-(4-methylphenyl)-5-[(5-oxo-4,5-dihydro-1,2,4-oxadiazol-3-yl)methyl]pyridin-3-yl}methyl)carbamate according to a method similar to the method of Example 2-3).

¹H-NMR (DMSO-d₆) δ:0.98 (6H, d, J=6.6 Hz), 2.13-2.21 (1H, m), 2.39 (3H, s), 2.75 (3H, s), 3.05 (2H, brs), 3.66 (2H, s), 3.76 (2H, brs), 7.16 (2H, d, J=7.8 Hz), 7.36 (2H, d, J=7.8 Hz), 8.26 (3H, brs).

### Example 204

### diethyl {[5-(aminomethyl)-6-isobutyl-2-methyl-4-(4-methylphenyl)pyridin-3-yl]methyl}phosphonate dihydrochloride

1) Triethyl phosphite (772 μL, 4.5 mmol) was added to [5-{[(tert-butoxycarbonyl)amino]methyl}-6-isobutyl-2-methyl-4-(4-methylphenyl)pyridin-3-yl]methyl methanesulfonate (692 mg, 1.45 mmol) and the mixture was stirred at 150° C. for 3 hrs. The reaction mixture was allowed to cool to room temperature and purified by silica gel column chromatography to give diethyl {[5-{[(tert-butoxycarbonyl)amino]methyl}-6-isobutyl-2-methyl-4-(4-methylphenyl)pyridin-3-yl]methyl}phosphonate (314 mg, yield 42%) as a white powder.

¹H-NMR (CDCl₃) δ:0.95 (6H, d, J=6.6 Hz), 1.17 (6H, t, J=7.2 Hz), 1.38 (9H, s), 2.14-2.24 (1H, m), 2.40 (3H, s), 2.66 (3H, s), 2.73 (2H, d, J=5.1 Hz), 2.96 (1H, s), 3.04 (1H, s), 3.86 (4H, q, J=7.2 Hz), 4.00 (2H, d, J=4.8 Hz), 4.17 (1H, brs), 7.07 (2H, d, J=8.1 Hz), 7.24 (2H, d, J=8.1 Hz).

2) Diethyl {[5-(aminomethyl)-6-isobutyl-2-methyl-4-(4-methylphenyl)pyridin-3-yl]methyl}phosphonate dihydrochloride (106 mg, yield 96%) was obtained as a white powder from diethyl {[5-{[(tert-butoxycarbonyl)amino]methyl}-6-isobutyl-2-methyl-4-(4-methylphenyl)pyridin-3-yl]methyl}phosphonate according to a method similar to the method of Example 2-3).

¹H-NMR (DMSO-d₆) δ:0.97 (6H, d, J=6.3 Hz), 1.21 (6H, t, J=7.2 Hz), 2.11-2.18 (1H, m), 2.42 (3H, s), 2.95 (3H, s), 3.09 (2H, s), 3.17 (2H, s), 3.78 (2H, s), 3.82 (4H, q, J=7.2 Hz), 7.26 (2H, d, J=7.8 Hz), 7.39 (2H, d, J=7.8 Hz), 8.43 (3H, brs).

### Example 205

### pyridin-2-ylmethyl 5-(aminomethyl)-6-isobutyl-2-methyl-4-(4-methylphenyl)nicotinate trihydrochloride

1) Pyridin-2-ylmethyl 5-{[(tert-butoxycarbonyl)amino]methyl}-6-isobutyl-2-methyl-4-(4-methylphenyl)nicotinate (1.21 g, yield 99%) was obtained as a colorless oil from 5-{[(tert-butoxycarbonyl)amino]methyl}-6-isobutyl-2-methyl-4-(4-methylphenyl)nicotinic acid (1.00 g, 2.42 mmol), 2-(bromomethyl)pyridine hydrobromide (0.92 g, 3.64 mmol) and potassium carbonate (1.00 g, 7.27 mmol) according to a method similar to the method of Example 169-1).

¹H-NMR (CDCl₃)δ:0.97 (6H, d, J=6.6 Hz), 1.39 (9H, s), 2.14-2.25 (1H, m), 2.35 (3H, s), 2.56 (3H, s), 2.78 (2H, d, J=7.2 Hz), 4.14 (2H, brs), 4.25 (1H, brs), 5.06 (2H, s), 6.89 (1H, d, J=7.7 Hz), 7.06 (2H, d, J=7.9 Hz), 7.13 (2H, d, J=7.9 Hz), 7.17-7.22 (1H, m), 7.57 (1H, t, J=7.7 Hz), 8.52 (1H, d, J=4.7 Hz).

2) Pyridin-2-ylmethyl 5-(aminomethyl)-6-isobutyl-2-methyl-4-(4-methylphenyl)nicotinate trihydrochloride (1.23 g, yield 99%) was obtained as a white solid from pyridin-2-ylmethyl 5-{[(tert-butoxycarbonyl)amino]methyl}-6-isobutyl-2-methyl-4-(4-methylphenyl)nicotinate (1.21 g, 2.40 mmol) according to a method similar to the method of Example 2-3).

¹H-NMR (DMSO-d₆)δ:0.97 (6H, d, J=6.4 Hz), 2.17-2.28 (1H, m), 2.34 (3H, s), 2.61 (3H, s), 2.94 (2H, d, J=6.9 Hz), 3.81 (2H, d, J=4.9 Hz), 5.20 (2H, s), 7.19 (4H, s), 7.23 (1H, brs), 7.62-7.66 (1H, m), 8.06 (1H, t, J=7.9 Hz), 8.39 (3H, brs), 8.68 (1H, d, J=4.9 Hz).

### Example 206

### benzyl [5-(aminomethyl)-6-isobutyl-2-methyl-4-(4-methylphenyl)pyridin-3-yl]acetate dihydrochloride

1) Benzyl [5-{[(tert-butoxycarbonyl)amino]methyl}-6-isobutyl-2-methyl-4-(4-methylphenyl)pyridin-3-yl]acetate (305 mg, yield 84%) was obtained as a white powder from [5-{[(tert-butoxycarbonyl)amino]methyl}-6-isobutyl-2-methyl-4-(4-methylphenyl)pyridin-3-yl]acetic acid (300 mg, 0.703 mmol) and benzyl bromide (180 mg, 1.05 mmol) according to a method similar to the method of Example 169-1).

¹H-NMR (CDCl₃) δ:0.97 (6H, d, J=6.8 Hz), 1.38 (9H, s), 2.12-2.28 (1H, m), 2.38 (3H, s), 2.49 (3H, s), 2.76 (2H, d, J=6.6 Hz), 3.39 (2H, s), 4.03 (2H, d, J=5.1 Hz), 4.20 (1H, brs), 5.05 (2H, s), 6.90 (2H, d, J=7.9 Hz), 7.14 (2H, d, J=7.9 Hz), 7.19-7.25 (2H, m), 7.31-7.40 (3H, m).

2) Benzyl [5-(aminomethyl)-6-isobutyl-2-methyl-4-(4-methylphenyl)pyridin-3-yl]acetate dihydrochloride (214.5 mg, yield 95%) was obtained as a white powder from benzyl [5-{[(tert-butoxycarbonyl)amino]methyl}-6-isobutyl-2-methyl-4-(4-methylphenyl)pyridin-3-yl]acetate (240 mg, 0.464 mmol) according to a method similar to the method of Example 2-3).

¹H-NMR (DMSO-d₆)δ:0.98 (6H, d, J=6.6 Hz), 2.11-2.27 (1H, m), 2.38 (3H, s), 2.78 (3H, s), 3.15 (2H, s), 3.78 (2H, d, J=5.1 Hz), 5.04 (2H, s), 7.10 (2H, d, J=8.1 Hz), 7.20-7.45 (7H, m), 8.40 (3H, brs).

### Example 207

### 4-({[5-(aminomethyl)-6-isobutyl-2-methyl-4-(4-ethylphenyl)pyridin-3-yl]methyl}thio)benzamide dihydrochloride

1) tert-Butyl {[5-({[4-(aminocarbonyl)phenyl]thio}methyl)-2-isobutyl-6-methyl-4-(4-methylphenyl)pyridin-3-yl]methyl}carbamate (360 mg, yield 72%) was obtained as a white solid from 4-({[5-{[(tert-butoxycarbonyl)amino]methyl}-6-isobutyl-2-methyl-4-(4-methylphenyl)pyridin-3-yl]methyl}thio)benzoic acid (0.50 g, 0.935 mmol) according to a method similar to the method of Example 3-1).

¹H-NMR (CDCl₃) δ:0.97 (6H, d, J=6.6 Hz), 1.38 (9H, s), 2.13-2.25 (1H, m), 2.38 (3H, s), 2.65 (3H, s), 2.76 (2H, d, J=7.4 Hz), 3.85 (2H, s), 4.04 (2H, d, J=5.1 Hz), 4.20 (1H, brs), 7.05 (2H, d, J=7.4 Hz), 7.12 (2H, d, J=8.5 Hz), 7.19 (2H, d, J=7.9 Hz), 7.64 (2H, d, J=8.5 Hz).

2) 4-({[5-(Aminomethyl)-6-isobutyl-2-methyl-4-(4-methylphenyl)pyridin-3-yl]methyl}thio)benzamide dihydrochloride (253 mg, yield 74%) was obtained as a white solid from tert-butyl {[5-({[4-(aminocarbonyl)phenyl]thio}methyl)-2-isobutyl-6-methyl-4-(4-methylphenyl)pyridin-3-yl]methyl}carbamate (360 mg, 0.674 mmol) according to a method similar to the method of Example 2-3).

¹H-NMR (DMSO-d₆) δ:0.99 (6H, d, J=6.5 Hz), 2.13-2.22 (1H, m), 2.37 (3H, s), 2.86 (3H, brs), 3.14 (2H, brs), 3.78 (2H, d, J=4.7 Hz), 3.99 (2H, s), 7.22 (2H, d, J=8.5 Hz), 7.26 (2H, d, J=8.1 Hz), 7.33 (2H, d, J=8.5 Hz), 7.37 (1H, brs) 7.98 (1H, brs), 8.39 (3H, brs).

### Example 208

### methyl 2-({[5-(aminomethyl)-6-isobutyl-2-methyl-4-(4-methylphenyl)pyridin-3-yl]methyl}thio)benzoate dihydrochloride

1) Methyl 2-({[5-{[(tert-butoxycarbonyl)amino]methyl}-6-isobutyl-2-methyl-4-(4-methylphenyl)pyridin-3-yl]methyl}thio)benzoate (1.19 g, yield 86%) was obtained as a colorless oil from tert-butyl {[5-(hydroxymethyl)-2-isobutyl-6-methyl-4-(4-methylphenyl)pyridin-3-yl]methyl}carbamate (1.00 g, 2.51 mmol) and methyl 2-mercaptobenzoate (422 mg, 2.51 mmol) according to a method similar to the method of Example 183-1).

¹H-NMR (CDCl₃) δ:0.98 (6H, d, J=6.6 Hz), 1.39 (9H, s), 2.12-2.26 (1H, m), 2.35 (3H, s), 2.66 (3H, s), 2.75 (2H, d, J=7.4 Hz), 3.77 (2H, s), 3.89 (3H, s), 4.03 (2H, d, J=4.9 Hz), 4.19 (1H, brs), 7.05 (1H, d, J=8.1 Hz), 7.09-7.13 (3H, m), 7.17 (2H, d, J=8.1 Hz), 7.32-7.38 (1H, m), 7.93 (1H, dd, J=7.7, 1.5 Hz).

2) Methyl 2-({[5-(aminomethyl)-6-isobutyl-2-methyl-4-(4-methylphenyl)pyridin-3-yl]methyl}thio)benzoate dihydrochloride (165 mg, yield 91%) was obtained as a white solid from methyl 2-({[5-{[(tert-butoxycarbonyl)amino]methyl}-6-isobutyl-2-methyl-4-(4-methylphenyl)pyridin-3-yl]methyl}thio)benzoate (190 mg, 0.346 mmol) according to a method similar to the method of Example 2-3).

¹H-NMR (DMSO-d₆) δ:0.98 (6H, d, J=6.6 Hz), 2.13-2.25 (1H, m), 2.34 (3H, s), 2.77 (3H, brs), 2.98 (2H, brs), 3.69-3.76 (2H, m), 3.80 (3H, s), 3.87 (2H, s), 7.22-7.27 (4H, m), 7.31 (2H, d, J=8.5 Hz), 7.47-7.52 (1H, m), 7.87 (1H, dd, J=7.7, 1.5 Hz), 8.18 (3H, brs).

### Example 209

### 2-({[5-(aminomethyl)-6-isobutyl-2-methyl-4-(4-methylphenyl)pyridin-3-yl]methyl}thio)benzoic acid

1) 2-({[5-{[(tert-Butoxycarbonyl)amino]methyl}-6-isobutyl-2-methyl-4-(4-methylphenyl)pyridin-3-yl]methyl}thio)benzoic acid (0.86 g, yield 88%) was obtained as a white solid from methyl 2-({[5-{[(tert-butoxycarbonyl)amino]methyl}-6-isobutyl-2-methyl-4-(4-methylphenyl)pyridin-3-yl]methyl}thio)benzoate (1.00 g, 1.82 mmol) according to a method similar to the method of Example 9-1).

¹H-NMR (CDCl₃) δ:0.98 (6H, d, J=6.6 Hz), 1.38 (9H, s), 2.13-2.24 (1H, m), 2.37 (3H, brs), 2.73 (3H, brs), 2.90 (2H, d, J=7.0 Hz), 3.77 (2H, s), 4.05 (2H, d, J=4.5 Hz), 4.32 (1H, brs), 7.01-7.10 (3H, m), 7.16-7.21 (3H, m), 7.30-7.36 (1H, m), 7.94-7.97 (1H, m).

2) 2-({[5-(Aminomethyl)-6-isobutyl-2-methyl-4-(4-methylphenyl)pyridin-3-yl]methyl}thio)benzoic acid (274 mg, yield 99%) was obtained as a white solid from 2-({[5-{[(tert-butoxycarbonyl)amino]methyl}-6-isobutyl-2-methyl-4-(4-methylphenyl)pyridin-3-yl]methyl}thio)benzoic acid (0.29 g, 0.542 mmol) according to a method similar to the method of Example 2-3).

¹H-NMR (DMSO-d₆) δ:0.99 (6H, d, J=6.4 Hz), 2.15-2.24 (1H, m), 2.34 (3H, s), 2.81 (3H, brs), 3.03 (2H, brs), 3.66-3.85 (4H, m), 7.19-7.35 (6H, m), 7.44-7.50 (1H, m), 7.88 (1H, d, J=7.5 Hz), 8.23 (3H, brs).

### Example 210

### 2-({[5-(aminomethyl)-6-isobutyl-2-methyl-4-(4-methylphenyl)pyridin-3-yl]methyl}thio)benzamide dihydrochloride

1) tert-Butyl {[5-({[2-(aminocarbonyl)phenyl]thio}methyl)-2-isobutyl-6-methyl-4-(4-methylphenyl)pyridin-3-yl]methyl}carbamate (0.23 g, yield 48%) was obtained as a white solid from 2-({[5-{[(tert-butoxycarbonyl)amino]methyl}-6-isobutyl-2-methyl-4-(4-methylphenyl)pyridin-3-yl]methyl}thio)benzoic acid (0.48 g, 0.898 mmol) according to a method similar to the method of Example 3-1).

¹H-NMR (CDCl₃) δ:0.97 (6H, d, J=6.8 Hz), 1.39 (9H, s), 2.14-2.26 (1H, m), 2.40 (3H, s), 2.64 (3H, s), 2.75 (2H, d, J=7.4 Hz), 3.82 (2H, s), 4.00 (2H, d, J=5.3 Hz), 4.27 (1H, brs), 5.39 (1H, brs), 6.68 (1H, brs), 6.99 (2H, d, J=7.9 Hz), 7.19-7.34 (5H, m), 7.75-7.78 (1H, m).

2) 2-({[5-(Aminomethyl)-6-isobutyl-2-methyl-4-(4-methylphenyl)pyridin-3-yl]methyl}thio)benzamide dihydrochloride (218 mg, yield 99%) was obtained as a white solid from tert-butyl {[5-({[2-(aminocarbonyl)phenyl]thio}methyl)-2-isobutyl-6-methyl-4-(4-methylphenyl)pyridin-3-yl]methyl}carbamate (0.23 g, 0.431 mmol) according to a method similar to the method of Example 2-3).

¹H-NMR (DMSO-d₆) δ:0.99 (6H, d, J=6.6 Hz), 2.10-2.24 (1H, m), 2.38 (3H, s), 2.83 (3H, s), 3.18 (2H, brs), 3.79 (2H, d, J=5.1 Hz), 3.86 (2H, s), 7.16 (2H, d, J=7.7 Hz), 7.23-7.36 (6H, m), 7.42 (1H, brs), 7.48 (1H, dd, J=7.4, 1.4 Hz), 7.84 (1H, brs), 8.41 (3H, brs).

### Example 211

### methyl 3-({[5-(aminomethyl)-6-isobutyl-2-methyl-4-(4-methylphenyl)pyridin-3-yl]methyl}thio)benzoate dihydrochloride

1) Methyl 3-({[5-{[(tert-butoxycarbonyl)amino]methyl}-6-isobutyl-2-methyl-4-(4-methylphenyl)pyridin-3-yl]methyl}thio)benzoate (1.35 g, yield 82%) was obtained as a brown solid from tert-butyl {[5-(hydroxymethyl)-2-isobutyl-6-methyl-4-(4-methylphenyl)pyridin-3-yl]methyl}carbamate (1.20 g, 3.01 mmol) and methyl 3-mercaptobenzoate (507 mg, 3.01 mmol) according to a method similar to the method of Example 183-1).

¹H-NMR (CDCl₃) δ:0.97 (6H, d, J=6.6 Hz), 1.39 (9H, s), 2.15-2.24 (1H, m), 2.38 (3H, s), 2.64 (3H, s), 2.75 (2H, d, J=7.4 Hz), 3.83 (2H, s), 3.90 (3H, s), 4.02 (2H, d, J=5.1 Hz), 4.22 (1H, brs), 7.00 (2H, d, J=8.1 Hz), 7.18 (2H, d, J=7.7 Hz), 7.28-7.30 (1H, m), 7.76-7.79 (1H, m), 7.80-7.84 (1H, m).

2) Methyl 3-({[5-(aminomethyl)-6-isobutyl-2-methyl-4-(4-methylphenyl)pyridin-3-yl]methyl}thio)benzoate dihydrochloride (268 mg, yield 87%) was obtained as a white solid from methyl 3-({[5-{[(tert-butoxycarbonyl)amino]methyl}-6-isobutyl-2-methyl-4-(4-methylphenyl)pyridin-3-yl]methyl}thio)benzoate (324 mg, 0.590 mmol) according to a method similar to the method of Example 2-3).

¹H-NMR (DMSO-d₆) δ:0.97 (6H, d, J=6.6 Hz), 2.11-2.23 (1H, m), 2.36 (3H, s), 2.75 (3H, s), 2.97 (2H, brs), 3.74 (2H, d, J=4.5 Hz), 3.85 (3H, s), 3.96 (2H, s), 7.19 (2H, d, J=7.4 Hz), 7.29 (2H, d, J=7.9 Hz), 7.43 (2H, d, J=5.1 Hz), 7.65 (1H, s), 7.79-7.83 (1H, m), 8.18 (3H, brs).

### Example 212

### 3-({[5-(aminomethyl)-6-isobutyl-2-methyl-4-(4-methylphenyl)pyridin-3-yl]methyl}thio)benzoic acid dihydrochloride

1) 3-({[5-{[(tert-Butoxycarbonyl)amino]methyl}-6-isobutyl-2-methyl-4-(4-methylphenyl)pyridin-3-yl]methyl}thio)benzoic acid (0.73 g, yield 73%) was obtained as a white solid from methyl 3-({[5-{[(tert-butoxycarbonyl)amino]methyl}-6-isobutyl-2-methyl-4-(4-methylphenyl)pyridin-3-yl]methyl}thio)benzoate (0.90 g, 1.64 mmol) according to a method similar to the method of Example 9-1).

¹H-NMR (CDCl₃) δ:0.97 (6H, d, J=6.6 Hz), 1.39 (9H, s), 2.13-2.26 (1H, m), 2.38 (3H, s), 2.68 (3H, s), 2.79 (2H, d, J=7.0 Hz), 3.85 (2H, s), 4.04 (2H, d, J=4.9 Hz), 4.24 (1H, brs), 7.00 (2H, d, J=7.2 Hz), 7.19 (2H, d, J=7.9 Hz), 7.30-7.35 (2H, m), 7.84 (1H, brs), 7.89 (1H, brs).

2) 3-({[5-(Aminomethyl)-6-isobutyl-2-methyl-4-(4-methylphenyl)pyridin-3-yl]methyl}thio)benzoic acid dihydrochloride (167 mg, yield 80%) was obtained as a white solid from 3-({[5-{[(tert-butoxycarbonyl)amino]methyl}-6-isobutyl-2-methyl-4-(4-methylphenyl)pyridin-3-yl]methyl}thio)benzoic acid (0.22 g, 0.441 mmol) according to a method similar to the method of Example 2-3).

¹H-NMR (DMSO-d₆) δ:0.98 (6H, d, J=6.6 Hz), 2.11-2.22 (1H, m), 2.37 (3H, s), 2.84 (3H, brs), 3.10 (2H, brs), 3.76 (2H, d, J=5.1 Hz), 3.97 (2H, s), 7.21 (2H, d, J=7.9 Hz), 7.30 (2H, d, J=7.9 Hz), 7.41-7.42 (2H, m), 7.65 (1H, s), 8.38 (3H, brs).

### Example 213

### 3-({[5-(aminomethyl)-6-isobutyl-2-methyl-4-(4-methylphenyl)pyridin-3-yl]methyl}thio)benzamide dihydrochloride

1) tert-Butyl {[5-({[3-(aminocarbonyl)phenyl]thio}methyl)-2-isobutyl-6-methyl-4-(4-methylphenyl)pyridin-3-yl]methyl}carbamate (460 mg, yield 92%) was obtained as a white solid from 3-({[5-{[(tert-butoxycarbonyl)amino]methyl}-6-isobutyl-2-methyl-4-(4-methylphenyl)pyridin-3-yl]methyl}thio)benzoic acid (0.50 g, 0.935 mmol) according to a method similar to the method of Example 3-1).

¹H-NMR (CDCl₃) δ:0.97 (6H, d, J=6.6 Hz), 1.39 (9H, s), 2.16-2.27 (1H, m), 2.38 (3H, s), 2.65 (3H, s), 2.75 (2H, d, J=7.2 Hz), 3.84 (2H, s), 4.02 (2H, d, J=5.1 Hz), 4.24 (1H, brs), 6.99 (2H, d, J=7.9 Hz), 7.19 (2H, d, J=7.7 Hz), 7.25-7.31 (2H, m), 7.49-7.53 (1H, m), 7.56-7.59 (1H, m).

2) 3-({[5-(Aminomethyl)-6-isobutyl-2-methyl-4-(4-methylphenyl)pyridin-3-yl]methyl}thio)benzamide dihydrochloride (439 mg, quant.) was obtained as a white solid from tert-butyl {[5-({[3-(aminocarbonyl)phenyl]thio}methyl)-2-isobutyl-6-methyl-4-(4-methylphenyl)pyridin-3-yl]methyl}carbamate (460 mg, 0.862 mmol) according to a method similar to the method of Example 2-3).

¹H-NMR (DMSO-d₆) δ:0.99 (6H, d, J=6.6 Hz), 2.13-2.22 (1H, m), 2.38 (3H, s), 2.86 (3H, s), 3.19 (2H, d, J=6.6 Hz), 3.78 (2H, d, J=4.9 Hz), 3.98 (2H, s), 7.23 (2H, d, J=8.1 Hz), 7.31-7.39 (4H, m), 7.45 (1H, brs), 7.70 (1H, brs), 7.75 (1H, d, J=7.4 Hz), 8.04 (1H, brs), 8.46 (3H, brs).

### Example 214

### 4-{[5-(aminomethyl)-6-isobutyl-2-methyl-4-(4-methylphenyl)pyridin-3-yl]methoxy}benzoic acid dihydrochloride

1) To a solution of tert-butyl {[5-(hydroxymethyl)-2-isobutyl-6-methyl-4-(4-methylphenyl)pyridin-3-yl]methyl}carbamate (0.50 g, 1.05 mmol), methyl 4-hydroxybenzoate (0.16 g, 1.05 mmol) and triphenylphosphine (0.36 g, 1.37 mmol) in tetrahydrofuran (10 mL) was added 40% solution (0.60 mL, 1.37 mmol) of diethyl azodicarboxylate in toluene and the mixture was stirred at room temperature for 30 min. The solvent was evaporated under reduced pressure and the obtained residue was purified by silica gel column chromatography to give methyl 4-{[5-{[(tert-butoxycarbonyl)amino]methyl}-6-isobutyl-2-methyl-4-(4-methylphenyl)pyridin-3-yl]methoxy}benzoate (380 mg, yield 68%) as a colorless oil.

¹H-NMR (CDCl₃) δ:0.99 (6H, d, J=6.6 Hz), 1.39 (9H, s), 2.16-2.27 (1H, m), 2.34 (3H, s), 2.62 (3H, s), 2.80 (2H, d, J=7.4 Hz), 3.87 (3H, s), 4.08-4.13 (2H, m), 4.30 (1H, brs), 4.68 (2H, s), 6.80 (2H, d, J=8.9 Hz), 7.04 (2H, d, J=7.9 Hz), 7.16 (2H, d, J=7.7 Hz), 7.93 (2H, d, J=8.9 Hz).

2) 4-{[5-{[(tert-Butoxycarbonyl)amino]methyl}-6-isobutyl-2-methyl-4-(4-methylphenyl)pyridin-3-yl]methoxy}benzoic acid (300 mg, yield 81%) was obtained as a white solid from methyl 4-{[5-{[(tert-butoxycarbonyl)amino]methyl}-6-isobutyl-2-methyl-4-(4-methylphenyl)pyridin-3-yl]methoxy}benzoate (380 mg, 0.713 mmol) according to a method similar to the method of Example 9-1).

¹H-NMR (CDCl₃) δ:1.00 (6H, d, J=6.6 Hz), 1.39 (9H, s), 2.17-2.29 (1H, m), 2.35 (3H, s), 2.66 (3H, brs), 2.84 (2H, brs), 4.08-4.14 (2H, m), 4.22-4.25 (1H, m), 4.70 (2H, s), 6.82 (2H, d, J=8.9 Hz), 7.04 (2H, d, J=7.9 Hz), 7.17 (2H, d, J=7.9 Hz), 7.99 (2H, d, J=8.9 Hz).

3) 4-{[5-(Aminomethyl)-6-isobutyl-2-methyl-4-(4-methylphenyl)pyridin-3-yl]methoxy}benzoic acid dihydrochloride (267 mg, yield 94%) was obtained as a white solid from 4-{(5-{[(tert-butoxycarbonyl)amino]methyl}-6-isobutyl-2-methyl-4-(4-methylphenyl)pyridin-3-yl]methoxy}benzoic acid (0.30 g, 0.578 mmol) according to a method similar to the method of Example 2-3).

¹H-NMR (CDCl₃) δ:1.00 (6H, d, J=6.6 Hz), 2.17-2.26 (1H, m), 2.34 (3H, s), 2.82 (3H, brs), 3.11 (2H, brs), 3.83 (2H, d, J=5.3 Hz), 4.79 (2H, s), 6.93 (2H, d, J=8.9 Hz), 7.26 (2H, d, J=8.1 Hz), 7.31 (2H, d, J=8.1 Hz), 7.85 (2H, d, J=8.9 Hz), 8.35 (3H, brs).

### Example 215

### methyl 4-{[5-(aminomethyl)-6-isobutyl-2-methyl-4-(4-methylphenyl)pyridin-3-yl]methoxy}benzoate dihydrochloride

Methyl 4-{[5-(aminomethyl)-6-isobutyl-2-methyl-4-(4-methylphenyl)pyridin-3-yl]methoxy}benzoate dihydrochloride (281 mg, yield 99%) was obtained as a white solid from methyl 4-{[5-{[(tert-butoxycarbonyl)amino]methyl}-6-isobutyl-2-methyl-4-(4-methylphenyl)pyridin-3-yl]methoxy}benzoate (0.30 mg, 0.563 mmol) according to a method similar to the method of Example 2-3).

¹H-NMR (DMSO-d₆) δ:1.00 (6H, d, J=6.6 Hz), 2.18-2.27 (1H, m), 2.33 (3H, s), 2.82 (3H, brs), 3.11 (2H, brs), 3.81-3.83 (5H, m), 4.80 (2H, s), 6.96 (2H, d, J=8.9 Hz), 7.26 (2H, d, J=7.9 Hz), 7.30 (2H, d, J=8.1 Hz), 7.87 (2H, d, J=8.9 Hz), 8.38 (3H, brs).

### Example 216

### {[2-isobutyl-6-methyl-4-(4-methylphenyl)pyridin-3-yl]methyl}amine dihydrochloride

1) To a solution of p-tolualdehyde (8.5 g, 78.3 mmol) and acetone (10 mL) in water (200 mL) was added sodium hydroxide (3.13 g, 78.3 mmol) and the mixture was stirred at room temperature for 3 days. The reaction mixture was diluted with ethyl acetate, washed successively with water and saturated brine and dried over anhydrous magnesium sulfate. The solvent was evaporated under reduced pressure to give 4-(4-methylphenyl)but-3-en-2-one (9.2 g, yield 80%) as an oil. The obtained oil (1.0 g, 6.24 mmol) was dissolved in ethanol (20 mL) and 3-amino-5-methylhex-2-enenitrile (0.93 g, 7.49 mmol) and sodium hydroxide (0.3 g, 7.49 mmol) were added. The mixture was heated under reflux for 2 hrs. The reaction mixture was diluted with ethyl acetate, washed successively with saturated aqueous ammonium chloride solution and saturated brine and dried over anhydrous magnesium sulfate. The solvent was evaporated under reduced pressure to give a residue. 2-Isobutyl-6-methyl-4-(4-methylphenyl)nicotinonitrile (0.45 g, yield 27%) was obtained as a yellow oil from the obtained residue according to a method similar to the method of Example 23-3).

¹H-NMR (CDCl₃) δ:1.01 (6H, d, J=6.6 Hz), 2.20-2.33 (1H, m), 2.43 (3H, s), 2.63 (3H, s), 2.96 (2H, d, J=7.4 Hz), 7.11 (1H, s), 7.31 (2H, d, J=7.9 Hz), 7.47 (2H, d, J=8.3 Hz).

2) {[2-Isobutyl-6-methyl-4-(4-methylphenyl)pyridin-3-yl]methyl}amine dihydrochloride (456 mg, yield 78%) was obtained as a white solid from 2-isobutyl-6-methyl-4-(4-methylphenyl)nicotinonitrile (0.45 g, 1.70 mmol) according to a method similar to the method of Example 108-3).

¹H-NMR (DMSO-d₆) δ:0.98 (6H, d, J=6.4 Hz), 2.13-2.22 (1H, m), 2.41 (3H, s), 2.72-2.82 (3H, m), 3.05-3.18 (2H, m), 4.02-4.11 (2H, m), 7.41 (4H, s), 7.67 (1H, brs), 8.47-8.58 (3H, m).

### Example 217

### ({2-isobutyl-6-methyl-4-(4-methylphenyl)-5-[(4-methylphenyl)sulfonyl]pyridin-3-yl}methyl)amine 4-methylbenzenesulfonate

1) To a solution of sodium 4-methylbenzenesulfinate (9.00 g, 50.5 mmol) in ethanol (50 mL) was added bromoacetone (6.9 g, 50 mmol) and the mixture was heated under reflux for 30 min. The reaction mixture was partitioned between ethyl acetate and water. The organic layer was washed with saturated brine and dried over anhydrous magnesium sulfate. The solvent was evaporated under reduced pressure and the residue was purified by silica gel column chromatography to give 1-[(4-methylphenyl)sulfonyl]acetone (8.0 g, yield 75%) as a colorless oil.

¹H-NMR (CDCl₃)δ: 2.41 (3H, s), 2.46 (3H, s), 4.14 (2H, s), 7.37 (2H, d, J=8.2 Hz), 7.77 (2H, d, J=8.2 Hz).

2) A mixture of 1-[(4-methylphenyl)sulfonyl]acetone (2.0 g, 9.4 mmol), p-tolualdehyde (1.1 g, 9.4 mmol), piperidine (0.093 mL, 0.94 mmol), acetic acid (0.11 mL, 1.9 mmol) and toluene (100 mL) was heated under reflux using a Dean-Stark trap for 3 hrs. The reaction mixture was allowed to cool to room temperature, washed with saturated brine and dried over anhydrous magnesium sulfate. The solvent was evaporated under reduced pressure to give 4-(4-methylphenyl)-3-[(4-methylphenyl)sulfonyl]but-3-en-2-one as a crude product (3.5 g). A mixture of the crude product (1.73 g), 3-amino-5-methylhex-2-enenitrile (0.65 g, 5.23 mmol) and ethanol (50 mL) was heated under reflux for 12 hrs. The reaction mixture was allowed to cool to room temperature, and the solvent was evaporated under reduced pressure. The residue was purified by silica gel column chromatography and the obtained solid was recrystallized from diisopropyl ether-ethyl acetate to give 2-isobutyl-6-methyl-4-(4-methylphenyl)-5-[(4-methylphenyl)sulfonyl]-1,4-dihydropyridine-3-carbonitrile (1.3 g, yield 64%) as a white powder.

melting point: 135-137° C.

3) 2-Isobutyl-6-methyl-4-(4-methylphenyl)-5-[(4-methylphenyl)sulfonyl]nicotinonitrile (0.77 g, yield 68%) was obtained as a white powder from 2-isobutyl-6-methyl-4-(4-methylphenyl)-5-[(4-methylphenyl)sulfonyl]-1,4-dihydropyridine-3-carbonitrile (1.1 g, 2.7 mmol) according to a method similar to the method of Example 23-3).

¹H-NMR (CDCl₃)δ: 0.99 (6H, d, J=6.6 Hz), 2.20-2.35 (1H, m), 2.38 (3H, s), 2.39 (3H, s), 2.91 (2H, d, J=7.2 Hz), 3.07 (3H, s), 6.86 (2H, d, J=8.1 Hz), 7.08 (4H, d, J=8.1 Hz), 7.23 (2H, d, J=8.1 Hz).

4) ({2-Isobutyl-6-methyl-4-(4-methylphenyl)-5-[(4-methylphenyl)sulfonyl]pyridin-3-yl}methyl)amine (0.64 g, yield 93%) was obtained as a colorless oil from 2-isobutyl-6-methyl-4-(4-methylphenyl)-5-[(4-methylphenyl)sulfonyl]nicotinonitrile (0.69 g, 1.6 mmol) according to a method similar to the method of Example 1-4).

¹H-NMR (CDCl₃)δ: 0.96 (6H, d, J=6.6 Hz), 1.41 (2H, brs), 2.20-2.35 (1H, m), 2.38 (6H, s), 2.79 (2H, d, J=7.2 Hz), 2.96 (3H, s), 3.40 (2H, s), 6.76 (2H, d, J=8.1 Hz), 7.03 (2H, d, J=8.3 Hz), 7.09 (2H, d, J=8.1 Hz), 7.27 (2H, d, J=8.3 Hz).

5) ({2-Isobutyl-6-methyl-4-(4-methylphenyl)-5-[(4-methylphenyl)sulfonyl]pyridin-3-yl}methyl)amine (0.64 g, 1.5 mmol) was dissolved in ethanol (5 mL) and a solution of p-toluenesulfonic acid hydrate (0.29 g, 1.5 mmol) in ethanol (5 mL) was added dropwise with stirring at room temperature. The mixture was stirred at room temperature for 10 min. The precipitate was collected by filtration, washed with cooled ethanol and dried to give ({2-isobutyl-6-methyl-4-(4-methylphenyl)-5-[(4-methylphenyl)sulfonyl]pyridin-3-yl}methyl)amine 4-methylbenzenesulfonate (0.57 g, yield 63%) as a white powder.

¹H-NMR (DMSO-d₆)δ: 0.94 (6H, d, J=6.6 Hz), 2.15-2.30 (1H, m), 2.29 (3H, s), 2.37 (6H, s), 2.78 (2H, d, J=7.0 Hz), 2.84 (3H, s), 3.57 (2H, s), 6.87 (2H, d, J=7.9 Hz), 7.11 (4H, d, J=8.5 Hz), 7.25-7.30 (4H, m), 7.47 (2H, d, J=7.9 Hz), 7.76 (3H, brs).

### Example 218

### {[2-isobutyl-6-methyl-4-(4-methylphenyl)-5-(methylsulfonyl)pyridin-3-yl]methyl}amine

1) A mixture of 1-(methylsulfonyl)acetone (3.68 g, 27 mmol), p-tolualdehyde (3.24 g, 27 mmol), piperidine (0.26 mL, 2.7 mmol), acetic acid (0.31 mL, 5.4 mmol) and toluene (200 mL) was heated under reflux using a Dean-Stark trap for 12 hrs. The reaction mixture was allowed to cool to room temperature, washed with saturated brine and dried over anhydrous magnesium sulfate. The solvent was evaporated under reduced pressure and the obtained residue was dissolved in methanol (20 mL). 3-Amino-5-methylhex-2-enenitrile (4.3 g, 35 mmol) was added and the mixture was heated under reflux for 6 hrs. The reaction mixture was concentrated under reduced pressure and the residue was purified by silica gel column chromatography to give 2-isobutyl-6-methyl-4-(4-methylphenyl)-5-(methylsulfonyl)-1,4-dihydropyridine-3-carbonitrile (6.38 g, yield 68%) as a yellow oil.

¹H-NMR (CDCl₃) δ:0.95 (3H, d, J=6.6 Hz), 1.01 (3H, d, J=6.6 Hz), 2.18-2.25 (1H, m), 2.32 (3H, s), 2.35 (3H, s), 2.40 (3H, s), 2.44 (1H, s), 3.04 (1H, s), 4.69 (1H, s), 5.80 (1H, s), 7.14 (2H, d, J=8.1 Hz), 7.21 (2H, d, J=8.3 Hz).

2) 2-Isobutyl-6-methyl-4-(4-methylphenyl)-5-(methylsulfonyl)nicotinonitrile (4.14 g, yield 65%) was obtained as a white solid from 2-isobutyl-6-methyl-4-(4-methylphenyl)-5-(methylsulfonyl)-1,4-dihydropyridine-3-carbonitrile (6.38 g, 18.6 mmol) according to a method similar to the method of Example 23-3).

¹H-NMR (CDCl₃) δ:1.02 (6H, d, J=6.8 Hz), 2.23-2.37 (1H, m), 2.44 (3H, s), 2.95 (2H, d, J=7.2 Hz), 3.05 (3H, s), 7.24 (2H, d, J=8.1 Hz), 7.33 (2H, d, J=7.9 Hz).

3) {[2-Isobutyl-6-methyl-4-(4-methylphenyl)-5-(methylsulfonyl)pyridin-3-yl]methyl}amine (0.81 g, yield 75%) was obtained as a white solid from 2-isobutyl-6-methyl-4-(4-methylphenyl)-5-(methylsulfonyl)nicotinonitrile (1.06 g, 3.09 mmol) according to a method similar to the method of Example 1-4).

¹H-NMR (CDCl₃) δ:0.99 (6H, d, J=6.8 Hz), 2.22-2.36 (1H, m), 2.43 (3H, s), 2.80 (3H, s), 2.82 (2H, d, J=7.4 Hz), 2.96 (3H, s), 3.50 (2H, s), 7.12 (2H, d, J=7.9 Hz), 7.26 (2H, d, J=7.7 Hz).

### Example 219

### methyl 3-{[5-(aminomethyl)-6-isobutyl-2-methyl-4-(4-methylphenyl)pyridin-3-yl]methoxy}benzoate dihydrochloride

1) Methyl 3-{[5-{[(tert-butoxycarbonyl)amino]methyl}-6-isobutyl-2-methyl-4-(4-methylphenyl)pyridin-3-yl]methoxy}benzoate (730 mg, yield 72%) was obtained as a colorless oil from tert-butyl {[5-(hydroxymethyl)-2-isobutyl-6-methyl-4-(4-methylphenyl)pyridin-3-yl]methyl}carbamate (0.75 g, 1.89 mmol) and methyl 3-hydroxybenzoate (0.29 g, 1.90 mmol) according to a method similar to the method of Example 214-1).

¹H-NMR (CDCl₃) δ:0.99 (6H, d, J=6.6 Hz), 1.39 (9H, s), 2.19-2.28 (1H, m), 2.35 (3H, s), 2.62 (3H, s), 2.79 (2H, d, J=7.2 Hz), 3.89 (3H, s), 4.07-4.11 (2H, m), 4.67 (2H, s), 6.98-7.02 (1H, m), 7.05 (2H, d, J=7.9 Hz), 7.16 (2H, d, J=7.7 Hz), 7.29-7.32 (1H, m), 7.42-7.43 (1H, m), 7.60-7.63 (1H, m).

2) Methyl 3-{[5-(aminomethyl)-6-isobutyl-2-methyl-4-(4-methylphenyl)pyridin-3-yl]methoxy}benzoate dihydrochloride (116 mg, yield 85%) was obtained as a white solid from methyl-3-{[5-{[(tert-butoxycarbonyl)amino]methyl}-6-isobutyl-2-methyl-4-(4-methylphenyl)pyridin-3-yl]methoxy}benzoate (144 mg, 0.270 mmol) according to a method similar to the method of Example 2-3).

¹H-NMR (DMSO-d₆) δ:1.00 (6H, d, J=6.6 Hz), 2.17-2.26 (1H, m), 2.34 (3H, s), 2.83 (3H, brs), 3.11 (2H, brs), 3.83 (5H, s), 4.79 (2H, s), 7.15 (1H, dd, J=7.8, 2.2 Hz), 7.27 (2H, d, J=8.3 Hz), 7.29-7.35 (3H, m), 7.42 (2H, t, J=7.9 Hz), 7.56 (1H, d, J=7.7 Hz), 8.38 (3H, brs).

### Example 220

### 3-{[5-(aminomethyl)-6-isobutyl-2-methyl-4-(4-methylphenyl)pyridin-3-yl]methoxy}benzoic acid dihydrochloride

1) 3-{[5-{[(tert-Butoxycarbonyl)amino]methyl}-6-isobutyl-2-methyl-4-(4-methylphenyl)pyridin-3-yl]methoxy}benzoic acid (460 mg, yield 80%) was obtained as a colorless oil from methyl 3-{[5-{[(tert-butoxycarbonyl)amino]methyl}-6-isobutyl-2-methyl-4-(4-methylphenyl)pyridin-3-yl]methoxy}benzoate (0.58 g, 1.10 mmol) according to a method similar to the method of Example 9-1).

¹H-NMR (CDCl₃) δ:0.99 (6H, d, J=6.6 Hz), 1.39 (9H, s), 2.17-2.28 (1H, m), 2.34 (3H, s), 2.65 (3H, s), 2.82 (2H, d, J=7.2 Hz), 4.11 (2H, brs), 4.28 (1H, brs), 4.68 (2H, s), 7.03-7.07 (3H, m), 7.16 (2H, d, J=7.9 Hz), 7.33 (1H, t, J=8.0 Hz), 7.47 (1H, brs), 7.64-7.70 (1H, m).

2) 3-{[5-(Aminomethyl)-6-isobutyl-2-methyl-4-(4-methylphenyl)pyridin-3-yl]methoxy}benzoic acid dihydrochloride (128 mg, yield 99%) was obtained as a white solid from 3-{[5-{[(tert-butoxycarbonyl)amino]methyl}-6-isobutyl-2-methyl-4-(4-methylphenyl)pyridin-3-yl]methoxy}benzoic acid (136 mg, 0.262 mmol) according to a method similar to the method of Example 2-3).

¹H-NMR (DMSO-d₆) δ:1.00 (6H, d, J=6.2 Hz), 2.18-2.27 (1H, m), 2.34 (3H, s), 2.73-2.79 (3H, m), 3.04 (2H, brs), 3.81 (2H, brs), 4.76 (2H, s), 7.11 (2H, d, J=8.1 Hz), 7.21-7.31 (5H, m), 7.38 (1H, t, J=7.7 Hz), 7.54 (1H, d, J=7.5 Hz), 8.27 (3H, brs).

### Example 221

### methyl 2-{[5-(aminomethyl)-6-isobutyl-2-methyl-4-(4-methylphenyl)pyridin-3-yl]methoxy}benzoate dihydrochloride

1) Methyl 2-{[5-{[(tert-butoxycarbonyl)amino]methyl}-6-isobutyl-2-methyl-4-(4-methylphenyl)pyridin-3-yl]methoxy}benzoate (700 mg, yield 70%) was obtained as a white solid from tert-butyl {[5-(hydroxymethyl)-2-isobutyl-6-methyl-4-(4-methylphenyl)pyridin-3-yl]methyl}carbamate (0.75 g, 1.89 mmol) and methyl 2-hydroxybenzoate (0.29 g, 1.90 mmol) according to a method similar to the method of Example 214-1).

¹H-NMR (CDCl₃) δ:0.99 (6H, d, J=6.6 Hz), 1.39 (9H, s), 2.19-2.28 (1H, m), 2.36 (3H, s), 2.67 (3H, s), 2.78 (2H, d, J=7.4 Hz), 3.81 (3H, s), 4.09 (2H, d, J=4.0 Hz), 4.23 (1H, brs), 4.71 (2H, s), 6.66 (1H, d, J=8.3 Hz), 6.93-6.98 (1H, m), 7.04 (2H, d, J=8.1 Hz), 7.16 (2H, d, J=7.7 Hz), 7.29-7.35 (1H, m), 7.72 (1H, dd, J=7.6, 1.8 Hz).

2) Methyl 2-{[5-(aminomethyl)-6-isobutyl-2-methyl-4-(4-methylphenyl)pyridin-3-yl]methoxy}benzoate dihydrochloride (42.3 mg, yield 56%) was obtained as a white solid from methyl 2-{[5-{[(tert-butoxycarbonyl)amino]methyl}-6-isobutyl-2-methyl-4-(4-methylphenyl)pyridin-3-yl]methoxy}benzoate (78.8 mg, 0.148 mmol) according to a method similar to the method of Example 2-3).

¹H-NMR (DMSO-d₆) δ:1.00 (6H, d, J=6.6 Hz), 2.18-2.29 (1H, m), 2.36 (3H, s), 2.83 (3H, brs), 3.07 (2H, brs), 3.74 (3H, s), 3.83 (2H, d, J=4.7 Hz), 4.78 (2H, s), 6.91 (1H, d, J=8.5 Hz), 7.03 (2H, t, J=7.4 Hz), 7.25 (2H, d, J=7.9 Hz), 7.30 (2H, d, J=8.1 Hz), 7.42-7.48 (1H, m), 7.64 (1H, dd, J=7.6, 1.6 Hz), 8.30 (3H, brs).

### Example 222

### 2-{[5-(aminomethyl)-6-isobutyl-2-methyl-4-(4-methylphenyl)pyridin-3-yl]methoxy}benzoic acid dihydrochloride

1) 2-{[5-{[(tert-Butoxycarbonyl)amino]methyl}-6-isobutyl-2-methyl-4-(4-methylphenyl)pyridin-3-yl]methoxy}benzoic acid (140 mg, yield 23%) was obtained as a white solid from methyl 2-{[5-{[(tert-butoxycarbonyl)amino]methyl}-6-isobutyl-2-methyl-4-(4-methylphenyl)pyridin-3-yl]methoxy}benzoate (0.62 g, 1.17 mmol) according to a method similar to the method of Example 9-1).

¹H-NMR (CDCl₃) δ:0.99 (6H, d, J=6.6 Hz), 1.39 (9H, s), 2.21-2.30 (1H, m), 2.34 (3H, s), 2.65 (3H, s), 2.81 (2H, d, J=7.4 Hz), 4.10 (2H, d, J=5.3 Hz), 4.92 (2H, s), 6.83 (1H, d, J=8.3 Hz), 7.01 (2H, d, J=8.1 Hz), 7.10-7.15 (1H, m), 7.17 (2H, d, J=7.7 Hz), 7.44-7.50 (1H, m), 8.17 (1H, dd, J=7.8, 1.8 Hz).

2) 2-{[5-(Aminomethyl)-6-isobutyl-2-methyl-4-(4-methylphenyl)pyridin-3-yl]methoxy}benzoic acid dihydrochloride (103 mg, yield 77%) was obtained as a white solid from 2-{[5-{[(tert-butoxycarbonyl)amino]methyl}-6-isobutyl-2-methyl-4-(4-methylphenyl)pyridin-3-yl]methoxy}benzoic acid (0.14 g, 0.270 mmol) according to a method similar to the method of Example 2-3).

¹H-NMR (DMSO-d₆) δ:1.00 (6H, d, J=6.6 Hz), 2.18-2.27 (1H, m), 2.37 (3H, s), 2.89 (3H, brs), 3.13 (2H, brs), 3.84 (2H, d, J=4.7 Hz), 4.78 (2H, s), 6.86 (1H, d, J=8.5 Hz), 7.02 (1H, t, J=7.4 Hz), 7.27 (2H, d, J=7.9 Hz), 7.32 (2H, d, J=8.1 Hz), 7.38-7.44 (1H, m), 7.61 (1H, dd, J=7.5, 1.7 Hz), 8.39 (3H, brs).

### Example 223

### N-[5-(aminomethyl)-6-isobutyl-2-methyl-4-(4-methylphenyl)pyridin-3-yl]benzamide dihydrochloride

To a solution of tert-butyl {[5-amino-2-isobutyl-6-methyl-4-(4-methylphenyl)pyridin-3-yl]methyl}carbamate (192 mg, 0.5 mmol) in tetrahydrofuran (3 mL) was added benzoyl chloride (88 μL, 0.75 mmol) and triethylamine (140 μL, 1.0 mmol) was added. The mixture was stirred for 30 min. Saturated aqueous sodium hydroxide solution (5 mL) was added to the reaction mixture and the mixture was extracted with ethyl acetate. The organic layer was washed with saturated brine and dried over anhydrous magnesium sulfate. The solvent was evaporated under reduced pressure and the obtained residue was purified by silica gel column chromatography to give an oil. To a solution of the obtained oil in ethyl acetate (1 mL) was added 4N hydrogen chloride ethyl acetate solution (1 mL) and the mixture was stirred at room temperature for 1 hr. The solvent was evaporated under reduced pressure and the obtained residue was crystallized from hexane to give N-[5-(aminomethyl)-6-isobutyl-2-methyl-4-(4-methylphenyl)pyridin-3-yl]benzamide dihydrochloride (203 mg, yield 96%) as a white powder.

¹H-NMR (DOSO-d₆)δ:1.00 (6H, d, J=6.6 Hz), 2.20-2.32 (1H, m), 2.31 (3H, s), 2.64 (3H, s), 3.11 (2H, s), 3.87 (2H, s), 7.17-7.66 (9H, m), 8.49 (3H, brs), 10.13 (1H, brs).

### Example 224

### N-[5-(aminomethyl)-6-isobutyl-2-methyl-4-(4-methylphenyl)pyridin-3-yl]-2-phenylacetamide dihydrochloride

N-[5-(Aminomethyl)-6-isobutyl-2-methyl-4-(4-methylphenyl)pyridin-3-yl]-2-phenylacetamide dihydrochloride (208 mg, yield 95%) was obtained as a white powder from tert-butyl {[5-amino-2-isobutyl-6-methyl-4-(4-methylphenyl)pyridin-3-yl]methyl}carbamate (192 mg, 0.5 mmol) and phenylacetyl chloride (100 μL, 0.75 mmol) according to a method similar to the method of Example 223.

¹H-NMR (DMSO-d₆) δ:0.97 (6H, d, J=6.6 Hz), 1.98-2.26 (1H, m), 2.40 (3H, s), 2.50 (3H, s), 3.04 (2H, s), 3.40 (2H, s), 3.78 (2H, s), 6.94-6.97 (2H, m), 7.12-7.53 (7H, m), 8.44 (3H, brs), 9.90 (1H, brs).

### Example 225

### N-[5-(aminomethyl)-6-isobutyl-2-methyl-4-(4-methylphenyl)pyridin-3-yl]-3-phenylpropanamide dihydrochloride

N-[5-(Aminomethyl)-6-isobutyl-2-methyl-4-(4-methylphenyl)pyridin-3-yl]-3-phenylpropanamide dihydrochloride (208 mg, yield 92%) was obtained as a white powder from tert-butyl {[5-amino-2-isobutyl-6-methyl-4-(4-methylphenyl)pyridin-3-yl]methyl}carbamate (192 mg, 0.5 mmol) and hydrocinnamoyl chloride (111 μL, 0.75 mmol) according to a method similar to the method of Example 223.

¹H-NMR (DMSO-d₆) δ:0.97 (6H, d, J=6.6 Hz), 2.15-2.23 (1H, m), 2.33 (2H, t, J=7.2 Hz), 2.37 (6H, s), 2.63 (2H, t, J=7.2 Hz), 2.94 (2H, brs), 3.79 (2H, s), 7.10-7.29 (9H, m), 8.26 (3H, brs), 9.43 (1H, brs).

### Example 226

### (2E)-N-[5-(aminomethyl)-6-isobutyl-2-methyl-4-(4-methylphenyl)pyridin-3-yl]-3-phenylacrylamide dihydrochloride

(2E)-N-[5-(Aminomethyl)-6-isobutyl-2-methyl-4-(4-methylphenyl)pyridin-3-yl]-3-phenylacrylamide dihydrochloride (208 mg, yield 92%) was obtained as a white powder from tert-butyl {[5-amino-2-isobutyl-6-methyl-4-(4-methylphenyl)pyridin-3-yl]methyl}carbamate (192 mg, 0.5 mmol) and cinnamoyl chloride (125 mg, 0.75 mmol) according to a method similar to the method of Example 223.

¹H-NMR (DMSO-d₆) δ:1.00 (6H, d, J=6.6 Hz), 2.15-2.28 (1H, m), 2.34 (3H, s), 2.55 (3H, s), 3.02 (2H, brs), 3.83 (2H, brs), 6.63 (1H, d, J=15.6 Hz), 7.16-7.23 (2H, m), 7.28-7.32 (2H, m), 7.39-7.46 (4H, m), 7.52-7.56 (2H, m), 8.36 (3H, brs), 9.76 (1H, brs).

### Example 227

### ethyl [({[5-(aminomethyl)-6-isobutyl-2-methyl-4-(4-methylphenyl) pyridin-3-yl]amino}carbonyl)oxy]acetate dihydrochloride

1) Ethyl [({[5-{[(tert-butoxycarbonyl)amino]methyl}-6-isobutyl-2-methyl-4-(4-methylphenyl)pyridin-3-yl]amino}carbonyl)oxy]acetate was obtained as an oil from 5-{[(tert-butoxycarbonyl)amino]methyl}-6-isobutyl-2-methyl-4-(4-methylphenyl)nicotinic acid (412 mg, 1.0 mmol) and ethyl hydroxyacetate (104 mg, 2.0 mmol) according to a method similar to the method of Example 95-1).

EIMS(M+1):514

2) Ethyl [({[5-(aminomethyl)-6-isobutyl-2-methyl-4-(4-methylphenyl)pyridin-3-yl]amino}carbonyl)oxy]acetate dihydrochloride (202 mg, yield 45%) was obtained as a white powder from the oil obtained in the aforementioned 1), according to a method similar to the method of Example 2-3).

¹H-NMR (DMSO-d₆)δ:0.96 (6H, d, J=6.3 Hz), 1.18 (3H, t, J=7.2 Hz), 2.11-2.29 (1H, m), 2.38 (3H, s), 2.86 (3H, s), 3.77 (2H, brs), 3.91 (2H, brs), 4.12 (2H, q, J=7.2 Hz), 4.52 (2H, s), 7.15 (2H, d, J=7.8 Hz), 7.29 (2H, d, J=7.8 Hz), 8.21 (3H, brs), 9.12 (1H, brs).

### Example 228

### N-[5-(aminomethyl)-6-isobutyl-2-methyl-4-(4-methylphenyl)pyridin-3-yl]-N′-benzylurea dihydrochloride

1) tert-Butyl {[5-{[(benzylamino)carbonyl]amino}-2-isobutyl-6-methyl-4-(4-methylphenyl)pyridin-3-yl]methyl}carbamate was obtained as an oil from 5-{[(tert-butoxycarbonyl)amino]methyl}-6-isobutyl-2-methyl-4-(4-methylphenyl)nicotinic acid (412 mg, 1.0 mmol) and benzylamine (218 μL, 2.0 mmol) according to a method similar to the method of Example 95-1).

EIMS(M+1):517

2) N-[5-(Aminomethyl)-6-isobutyl-2-methyl-4-(4-methylphenyl)pyridin-3-yl]-N′-benzylurea dihydrochloride (181 mg, yield 40%) was obtained as a white powder from the oil obtained in the aforementioned 1), according to a method similar to the method of Example 2-3).

¹H-NMR (DMSO-d₆) δ:0.96 (6H, d, J=6.3 Hz), 2.09-2.22 (1H, m), 2.41 (3H, s), 2.50 (3H, s), 2.65 (2H, brs), 3.81 (2H, brs), 4.19(2H, brs), 7.11-7.35 (9H, m), 8.43 (3H, brs).

### Example 229

### methyl 4-{[({[5-(aminomethyl)-6-isobutyl-2-methyl-4-(4-methylphenyl)pyridin-3-yl]amino}carbonyl)oxy]methyl}benzoate dihydrochloride

1) Methyl 4-{[({[5-{[(tert-butoxycarbonyl)amino]methyl}-6-isobutyl-2-methyl-4-(4-methylphenyl)pyridin-3-yl]amino}carbonyl)oxy]methyl}benzoate was obtained as an oil from 5-{[(tert-butoxycarbonyl)amino]methyl}-6-isobutyl-2-methyl-4-(4-methylphenyl)nicotinic acid (412 mg, 1.0 mmol) and methyl 4-hydroxymethylbenzoate (250 mg, 1.5 mmol) according to a method similar to the method of Example 95-1).

EIMS(M+1):576

2) Methyl 4-{[({[5-(aminomethyl)-6-isobutyl-2-methyl-4-(4-methylphenyl)pyridin-3-yl]amino}carbonyl)oxy]methyl}benzoate dihydrochloride (195 mg, yield 38%) was obtained as a white powder from the oil obtained in the aforementioned 1), according to a method similar to the method of Example 2-3).

¹H-NMR (DMSO-d₆)δ:0.97 (6H, d, J=6.3 Hz), 2.14-2.23 (1H, m), 2.39 (3H, s), 2.55 (3H, s), 2.97 (2H, brs), 3.78 (2H, brs), 3.87 (3H, s), 5.09 (2H, brs), 7.14-7.29 (6H, m), 7.92 (2H, d, J=8.4 Hz), 8.30 (3H, brs), 9.19 (1H, brs).

### Example 230

### 3-[({[5-(aminomethyl)-6-isobutyl-2-methyl-4-(4-methylphenyl)pyridin-3-yl]carbonyl}oxy)methyl]benzoic acid dihydrochloride

1) To a solution of 5-{[(tert-butoxycarbonyl)amino]methyl}-6-isobutyl-2-methyl-4-(4-methylphenyl)nicotinic acid (1.70 g, 4.12 mmol) in N,N-dimethylformamide (15 mL) were added methyl 3-(bromomethyl)benzoate (0.79 g, 3.43 mmol) and potassium carbonate (0.71 g, 5.15 mmol) and the mixture was stirred at room temperature for 1 hr. The reaction mixture was diluted with ethyl acetate, and the mixture was washed with saturated brine and dried over anhydrous magnesium sulfate. The solvent was evaporated under reduced pressure and the obtained residue was purified by silica gel column chromatography to give 3-(methoxycarbonyl)benzyl 5-{[(tert-butoxycarbonyl)amino]methyl}-6-isobutyl-2-methyl-4-(4-methylphenyl)nicotinate (1.80 g, yield 94%) as a colorless oil.

¹H-NMR (CDCl₃) δ:0.96 (6H, d, J=6.6 Hz), 1.38 (9H, s), 2.16-2.25 (1H, m), 2.33 (3H, s), 2.53 (3H, s), 2.77 (2H, d, J=7.4 Hz), 3.94 (3H, s), 4.13 (2H, brs), 4.20 (1H, brs), 4.95 (2H, s), 7.01 (2H, d, J=8.1 Hz), 7.09 (2H, d, J=7.9 Hz), 7.22 (1H, d, J=7.7 Hz), 7.35 (1H, t. J=7.7 Hz), 7.83 (1H, s), 7.98 (1H, d, J=7.7 Hz).

2) 3-[({[5-{[(tert-Butoxycarbonyl)amino]methyl}-6-isobutyl-2-methyl-4-(4-methylphenyl)pyridin-3-yl]carbonyl}oxy)methyl]benzoic acid (1.43 g, yield 87%) was obtained as a colorless oil from 3-(methoxycarbonyl)benzyl 5-{[(tert-butoxycarbonyl)amino]methyl}-6-isobutyl-2-methyl-4-(4-methylphenyl)nicotinate (1.69 g, 3.01 mmol) according to a method similar to the method of Example 9-1).

¹H-NMR (CDCl₃) δ:0.96 (6H, d, J=6.6 Hz), 1.38 (9H, s), 2.13-2.25 (1H, m), 2.34 (3H, s), 2.55 (3H, s), 2.80 (2H, d, J=7.4 Hz), 4.11-4.16 (2H, m), 4.22 (1H, brs), 4.98 (2H, s), 7.02 (2H, d, J=7.9 Hz), 7.11 (2H, d, J=7.7 Hz), 7.26-7.30 (1H, m), 7.39 (1H, t. J=7.7 Hz), 7.89 (1H, s), 8.04 (1H, d, J=7.5 Hz).

3) 3-[({[5-(Aminomethyl)-6-isobutyl-2-methyl-4-(4-methylphenyl)pyridin-3-yl]carbonyl}oxy)methyl]benzoic acid dihydrochloride (293 mg, yield 60%) was obtained as a white solid from 3-[({[5-{[(tert-butoxycarbonyl)amino]methyl}-6-isobutyl-2-methyl-4-(4-methylphenyl)pyridin-3-yl]carbonyl}oxy)methyl]benzoic acid (0.50 g, 0.927 mmol) according to a method similar to the method of Example 2-3).

¹H-NMR (DMSO-d₆) δ:0.96 (6H, d, J=6.6 Hz), 2.16-2.25 (1H, m), 2.32 (3H, s), 2.54 (3H, s), 2.90 (2H, d, J=6.6 Hz), 3.81 (2H, d, J=5.1 Hz), 5.04 (2H, s), 7.13 (2H, d, J=8.5 Hz), 7.17 (2H, d, J=8.3 Hz), 7.26-7.30 (1H, m), 7.44 (1H, t. J=7.6 Hz), 7.73-7.74 (1H, m), 7.89-7.92 (1H, m), 8.30 (3H, brs).

### Example 231

### 2-[({[5-(aminomethyl)-6-isobutyl-2-methyl-4-(4-methylphenyl)pyridin-3-yl]carbonyl}oxy)methyl]benzoic acid dihydrochloride

1) To a solution of 5-{[(tert-butoxycarbonyl)amino]methyl}-6-isobutyl-2-methyl-4-(4-methylphenyl)nicotinic acid (1.10 g, 2.67 mmol) in N,N-dimethylformamide (15 mL) were added 2-bromobenzyl bromide (0.61 g, 2.43 mmol) and potassium carbonate (0.51 g, 3.65 mmol) and the mixture was stirred at room temperature for 1 hr. The reaction mixture was diluted with ethyl acetate, washed with saturated brine and dried over anhydrous magnesium sulfate. The solvent was evaporated under reduced pressure and the obtained residue was purified by silica gel column chromatography to give 2-bromobenzyl 5-{[(tert-butoxycarbonyl)amino]methyl}-6-isobutyl-2-methyl-4-(4-methylphenyl)nicotinate (1.23 g, yield 87%) as a colorless oil.

¹H-NMR (CDCl₃) δ:0.97 (6H, d, J=6.8 Hz), 1.38 (9H, s), 2.14-2.25 (1H, m), 2.35 (3H, s), 2.56 (3H, s), 2.78 (2H, d, J=7.2 Hz), 4.11-4.13 (2H, m), 4.22 (1H, brs), 5.05 (2H, s), 7.02-7.05 (3H, m), 7.11 (2H, d, J=7.9 Hz), 7.16-7.21 (2H, m), 7.51-7.54 (1H, m).

2) 2-Bromobenzyl 5-{[(tert-butoxycarbonyl)amino]methyl}-6-isobutyl-2-methyl-4-(4-methylphenyl)nicotinate (1.23 g, 2.12 mmol), triethylamine (0.59 mL, 4.24 mmol) and [1,1′-bis(diphenylphosphino)ferrocene]palladium(II) dichloride (174 mg, 0.212 mmol) were dissolved in methanol (5 mL)-N,N-dimethylformamide (15 mL) and the resulting mixture was stirred under a carbon monoxide atmosphere for 14 hrs. The reaction mixture was diluted with ethyl acetate (100 mL) and the mixture was washed with saturated brine. The organic layer was dried over anhydrous magnesium sulfate and the solvent was evaporated under reduced pressure. The obtained residue was purified by silica gel column chromatography to give 2-(methoxycarbonyl)benzyl 5-{[(tert-butoxycarbonyl)amino]methyl}-6-isobutyl-2-methyl-4-(4-methylphenyl)nicotinate (0.88 g, yield 74%) was obtained as a yellow oil.

¹H-NMR (CDCl₃) δ:0.97 (6H, d, J=6.6 Hz), 1.38 (9H, s), 2.16-2.25 (1H, m), 2.35 (3H, s), 2.56 (3H, s), 2.78 (2H, d, J=7.2 Hz), 3.87 (3H, s), 4.11-4.16 (2H, m), 4.21 (1H, brs), 5.39 (2H, s), 7.01-7.06 (3H, m), 7.11 (2H, d, J=7.9 Hz), 7.32-7.42 (2H, m), 7.93-7.96 (1H, m).

3) 2-[({[5-{[(tert-Butoxycarbonyl)amino]methyl}-6-isobutyl-2-methyl-4-(4-methylphenyl)pyridin-3-yl]carbonyl}oxy)methyl]benzoic acid (0.75 g, yield 89%) was obtained as a colorless oil from 2-(methoxycarbonyl)benzyl 5-{[(tert-butoxycarbonyl)amino]methyl}-6-isobutyl-2-methyl-4-(4-methylphenyl)nicotinate (0.88 g, 1.54 mmol) according to a method similar to the method of Example 9-1).

¹H-NMR (CDCl₃) δ:0.96 (6H, d, J=6.6 Hz), 1.37 (9H, s), 2.12-2.21 (1H, m), 2.36 (3H, s), 2.54 (3H, s), 2.83 (2H, d, J=7.2 Hz), 4.13-4.18 (2H, m), 4.25 (1H, brs), 5.38 (2H, s), 7.01-7.04 (3H, m), 7.11 (2H, d, J=7.5 Hz), 7.38-7.46 (2H, m), 8.06-8.09 (1H, m).

4) 2-[({[5-(Aminomethyl)-6-isobutyl-2-methyl-4-(4-methylphenyl)pyridin-3-yl]carbonyl}oxy)methyl]benzoic acid dihydrochloride (278 mg, yield 65%) was obtained as a white solid from 2-[({[5-{[(tert-butoxycarbonyl)amino]methyl}-6-isobutyl-2-methyl-4-(4-methylphenyl)pyridin-3-yl]carbonyl}oxy)methyl]benzoic acid (0.45 g, 0.823 mmol) according to a method similar to the method of Example 2-3).

¹H-NMR (DMSO-d₆) δ:0.96 (6H, d, J=6.6 Hz), 2.18-2.27 (1H, m), 2.35 (3H, s), 2.84 (2H, d, J=7.2 Hz), 3.82 (2H, d, J=5.3 Hz), 5.32 (2H, s), 6.97-7.00 (1H, m), 7.18 (2H, d, J=8.3 Hz), 7.24 (2H, d, J=7.9 Hz), 7.41-7.51 (2H, m), 7.87-7.91 (1H, m), 8.19 (3H, brs).

### Example 232

### methyl 4-({[5-(aminomethyl)-6-isobutyl-2-methyl-4-(4-methylphenyl)pyridin-3-yl]amino}carbonyl)benzoate dihydrochloride

Methyl 4-({[5-(aminomethyl)-6-isobutyl-2-methyl-4-(4-methylphenyl)pyridin-3-yl]amino}carbonyl)benzoate dihydrochloride (230 mg, yield 89%) was obtained as a white powder from tert-butyl {[5-amino-2-isobutyl-6-methyl-4-(4-methylphenyl)pyridin-3-yl]methyl}carbamate (192 mg, 0.5 mmol) and terephthalic acid monomethyl ester chloride (149 mg, 0.75 mmol) according to a method similar to the method of Example 223.

¹H-NMR (DMSO-d₆) δ:1.00 (6H, d, J=6.6 Hz), 2.22-2.31 (1H, m), 2.31 (3H, s), 2.54 (3H, s), 2.95 (2H, brs), 3.85 (2H, brs), 3.87 (3H, s), 7.20-7.27 (4H, m), 7.72 (2H, d, J=8.4 Hz), 7.99 (2H, d, J=8.4 Hz), 8.26 (3H, brs), 10.13 (1H, brs).

### Example 233

### 4-({[5-(aminomethyl)-6-isobutyl-2-methyl-4-(4-methylphenyl)pyridin-3-yl]amino}carbonyl)benzoic acid dihydrochloride

1) 4-({[5-{[(tert-Butoxycarbonyl)amino]methyl}-6-isobutyl-2-methyl-4-(4-methylphenyl)pyridin-3-yl]amino}carbonyl)benzoic acid (248 mg, yield 98%) was obtained as a white powder from methyl 4-({[5-{[(tert-butoxycarbonyl)amino]methyl}-6-isobutyl-2-methyl-4-(4-methylphenyl)pyridin-3-yl]amino}carbonyl)benzoate (260 mg, 0.48 mmol) according to a method similar to the method of Example 36-1).

¹H-NMR (DMSO-d₆) δ:0.98 (6H, d, J=6.6 Hz), 1.35 (9H, s), 2.18-2.29 (1H, m), 2.29 (3H, s), 2.59 (3H, s), 2.88 (2H, brs), 3.99 (2H, brs), 7.14 (1H, s), 7.20 (4H, s), 7.70 (2H, d, J=8.4 Hz), 7.97 (2H, d, J=8.4 Hz), 10.13 (1H, brs).

2) 4-({[5-(Aminomethyl)-6-isobutyl-2-methyl-4-(4-methylphenyl)pyridin-3-yl]amino}carbonyl)benzoic acid dihydrochloride (230 mg, yield 99%) was obtained as a white powder from 4-({[5-{[(tert-butoxycarbonyl)amino]methyl}-6-isobutyl-2-methyl-4-(4-methylphenyl)pyridin-3-yl]amino}carbonyl)benzoic acid (248 mg, 0.47 mmol) according to a method similar to the method of Example 2-3).

¹H-NMR (DMSO-d₆) δ:1.00 (6H, d, J=6.6 Hz), 2.22-2.32 (1H, m), 2.31 (3H, s), 2.55 (3H, s), 2.96 (2H, brs), 3.83 (2H, brs), 7.20-7.27 (4H, m), 7.70 (2H, d, J=8.1 Hz), 7.96 (2H, d, J=8.1 Hz), 8.26 (3H, brs), 10.11 (1H, brs).

### Example 234

### methyl (4-{[5-(aminomethyl)-2-methyl-4-(4-methylphenyl)-6-neopentylpyridin-3-yl]methoxy}phenyl)acetate dihydrochloride

1) Methyl (4-{[5-{[(tert-butoxycarbonyl)amino]methyl}-2-methyl-4-(4-methylphenyl)-6-neopentylpyridin-3-yl]methoxy}phenyl)acetate (0.36 g, yield 61%) was obtained as a white powder from tert-butyl {[5-(hydroxymethyl)-6-methyl-4-(4-methylphenyl)-2-neopentylpyridin-3-yl]methyl}carbamate (0.44 g, 1.1 mmol) and methyl 4-hydroxyphenylacetate (0.18 g, 1.1 mmol) according to a method similar to the method of Example 214-1).

¹H-NMR (CDCl₃)δ: 1.03 (9H, s), 1.37 (9H, s), 2.36 (3H, s), 2.61 (3H, s), 2.87 (2H, s), 3.55 (2H, s), 3.68 (3H, s), 4.05-4.25 (3H, m), 4.59 (2H, s), 6.76 (2H, d, J=8.5 Hz), 7.05 (2H, d, J=8.5 Hz), 7.14 (2H, d, J=8.5 Hz), 7.17 (2H, d, J=8.5 Hz).

2) Methyl (4-{[5-(aminomethyl)-2-methyl-4-(4-methylphenyl)-6-neopentylpyridin-3-yl]methoxy}phenyl)acetate dihydrochloride (0.088 g, yield 74%) was obtained as a white powder from methyl (4-{[5-{[(tert-butoxycarbonyl)amino]methyl}-2-methyl-4-(4-methylphenyl)-6-neopentylpyridin-3-yl]methoxy}phenyl)acetate (0.13 g, 0.22 mmol) according to a method similar to the method of Example 2-3).

¹H-NMR (DMSO-d₆)δ: 1.04 (9H, s), 2.35 (3H, s), 2.77 (3H, brs), 3.14 (2H, brs), 3.58 (2H, d, J=7.0 Hz), 3.59 (3H, s), 3.87 (2H, s), 4.66 (2H, s), 6.80 (2H, d, J=8.7 Hz), 7.14 (2H, d, J=8.7 Hz), 7.25 (2H, d, J=7.7 Hz), 7.31 (2H, d, J=7.7 Hz), 8.20 (3H, brs).

### Example 235

### methyl 2-[5-(aminomethyl)-6-isobutyl-2-methyl-4-(4-methylphenyl)pyridin-3-yl]-1,3-oxazole-4-carboxylate dihydrochloride

1) Methyl N-{[5-cyano-6-isobutyl-2-methyl-4-(4-methylphenyl)pyridin-3-yl]carbonyl}serinate (5.37 g, yield 87%) was obtained as a colorless oil from 5-cyano-6-isobutyl-2-methyl-4-(4-methylphenyl)nicotinic acid (5.00 g, 11.2 mmol) and serine methyl ester hydrochloride (2.09 g, 13.4 mmol) according to a method similar to the method of Example 195-2).

¹H-NMR (CDCl₃) δ:0.97 (6H, d, J=5.7 Hz), 2.15-2.26 (1H, m), 2.38 (3H, s), 2.57 (3H, s), 2.80 (2H, d, J=7.0 Hz), 3.36-3.42 (1H, m), 3.61-3.69 (1H, m), 3.73 (3H, s), 4.19-4.29 (2H, m), 4.43-4.52 (2H, m), 5.03 (2H, s), 6.21 (1H, d, J=7.0 Hz), 7.12-7.17 (2H, m), 7.17-7.22 (2H, m), 7.29-7.38 (5H, m).

2) A solution of methyl N-{[5-cyano-6-isobutyl-2-methyl-4-(4-methylphenyl)pyridin-3-yl]carbonyl}serinate (5.37 g, 9.81 mmol) in dichloromethane (50 mL) was cooled to −78° C. and diethylaminosulfur trifluoride (1.72 mL, 11.8 mmol) was added. The mixture was stirred at the same temperature for 1 hr. Potassium carbonate (1.36 g, 14.7 mmol) was added and the mixture was stirred at room temperature for 30 min. The reaction mixture was diluted with ethyl acetate, washed with saturated aqueous sodium hydrogen carbonate and dried over anhydrous magnesium sulfate. The solvent was evaporated under reduced pressure and the residue was purified by silica gel column chromatography to give methyl 2-[5-cyano-6-isobutyl-2-methyl-4-(4-methylphenyl)pyridin-3-yl]-4,5-dihydro-1,3-oxazole-4-carboxylate (3.59 g, yield 69%) as a colorless oil.

¹H-NMR (CDCl₃) δ:0.95 (6H, d, J=6.6 Hz), 2.15-2.26 (1H, m), 2.37 (3H, s), 2.57 (3H, s), 2.81 (2H, d, J=7.2 Hz), 3.71 (3H, s), 4.11-4.16 (1H, m), 4.23 (2H, d, J=5.5 Hz), 4.33 (1H, dd, J=8.8, 7.4 Hz), 4.59-4.65 (1H, m), 5.03 (2H, s), 7.05 (2H, d, J=8.5 Hz), 7.13-7.21 (2H, m), 7.29-7.38 (5H, m).

3) A solution of methyl 2-[5-cyano-6-isobutyl-2-methyl-4-(4-methylphenyl)pyridin-3-yl]-4,5-dihydro-1,3-oxazole-4-carboxylate (0.83 g, 2.12 mmol) and 1,8-diazabicyclo[5.4.0]-7-undecene (1.11 mL, 7.42 mmol) in dichloromethane (10 mL) was cooled to 0° C. and bromotrichloromethane (0.73 mL, 7.42 mmol) was added. The mixture was stirred at the same temperature for 1 hr. The reaction mixture was diluted with ethyl acetate, washed with saturated aqueous ammonium chloride solution and dried over anhydrous magnesium sulfate. The solvent was evaporated under reduced pressure and the residue was purified by silica gel column chromatography to give methyl 2-[5-cyano-6-isobutyl-2-methyl-4-(4-methylphenyl)pyridin-3-yl]-1,3-oxazole-4-carboxylate (520 mg, yield 63%) as a colorless oil.

¹H-NMR (CDCl₃) δ:1.03 (6H, d, J=6.8 Hz), 2.24-2.34 (4H, m), 2.59 (3H, s), 3.00 (2H, d, J=7.4 Hz), 3.92 (3H, s), 7.11 (2H, d, J=8.5 Hz), 7.16 (2H, d, J=8.3 Hz), 8.08 (1H, s).

4) Methyl 2-[5-(aminomethyl)-6-isobutyl-2-methyl-4-(4-methylphenyl)pyridin-3-yl]-1,3-oxazole-4-carboxylate dihydrochloride (456 mg, yield 73%) was obtained as a white solid from methyl 2-[5-cyano-6-isobutyl-2-methyl-4-(4-methylphenyl)pyridin-3-yl]-1,3-oxazole-4-carboxylate (0.52 g, 1.34 mmol) according to a method similar to the method of Example 108-3).

¹H-NMR (DMSO-d₆) δ:1.00 (6H, d, J=6.6 Hz), 2.21-2.30 (4H, m), 2.45-2.48 (3H, m), 2.90-3.02 (2H, m), 3.78 (3H, s), 3.85 (2H, d, J=4.7 Hz), 7.11 (2H, dd, J=8.1, 2.1 Hz), 7.20 (2H, d, J=8.1 Hz), 8.30-8.47 (3H, m), 8.77 (1H, d, J=1.5 Hz).

### Example 236

### 2-(4-{[5-(aminomethyl)-2-methyl-4-(4-methylphenyl)-6-neopentylpyridin-3-yl]methoxy}phenyl)acetamide dihydrochloride

1) tert-Butyl {[5-{[4-(2-amino-2-oxoethyl)phenoxy]methyl}-6-methyl-4-(4-methylphenyl)-2-neopentylpyridin-3-yl]methyl}carbamate (0.14 g, yield 47%) was obtained as a white powder from tert-butyl {[5-(hydroxymethyl)-6-methyl-4-(4-methylphenyl)-2-neopentylpyridin-3-yl]methyl}carbamate (0.22 g, 0.53 mmol) and 4-hydroxyphenylacetamide (0.081 g, 0.53 mmol) according to a method similar to the method of Example 214-1).

¹H-NMR (CDCl₃)δ: 1.04 (9H, s), 1.37 (9H, s), 2.36 (3H, s), 2.62 (3H, s), 2.88 (2H, s), 3.51 (2H, s), 4.10-4.25 (3H, m), 4.61 (2H, s), 5.35 (2H, brs), 6.75-6.80 (2H, m), 7.05 (2H, d, J=7.9 Hz), 7.10-7.20 (4H, m).

2) 2-(4-{[5-(Aminomethyl)-2-methyl-4-(4-methylphenyl)-6-neopentylpyridin-3-yl]methoxy}phenyl)acetamide dihydrochloride (0.098 g, yield 92%) was obtained as a pale-yellow powder from tert-butyl {[5-{[4-(2-amino-2-oxoethyl)phenoxy]methyl}-6-methyl-4-(4-methylphenyl)-2-neopentylpyridin-3-yl]methyl}carbamate (0.11 g, 0.20 mmol) according to a method similar to the method of Example 2-3).

¹H-NMR (DMSO-d₆)δ: 1.05 (9H, s), 2.36 (3H, s), 2.79 (3H, brs), 3.05-3.25 (2H, m), 3.28 (2H, s), 3.88 (2H, brs), 4.66 (2H, s), 6.79 (2H, d, J=8.5 Hz), 6.83 (1H, brs), 7.14 (2H, d, J=8.5 Hz), 7.26 (2H, d, J=7.4 Hz), 7.33 (2H, d, J=7.4 Hz), 7.42 (1H, brs), 8.19 (3H, brs).

### Example 237

### methyl (4-{[5-(aminomethyl)-6-isobutyl-2-methyl-4-(4-methylphenyl)pyridin-3-yl]methoxy}phenyl)acetate

1) Methyl (4-{[5-{[(tert-butoxycarbonyl)amino]methyl}-6-isobutyl-2-methyl-4-(4-methylphenyl)pyridin-3-yl]methoxy}phenyl)acetate (570 mg, yield 83%) was obtained as a colorless oil from tert-butyl {[5-(hydroxymethyl)-2-isobutyl-6-methyl-4-(4-methylphenyl)pyridin-3-yl]methyl}carbamate (500 mg, 1.25 mmol) and methyl (4-hydroxyphenyl)acetate (250 mg, 1.51 mmol) according to a method similar to the method of Example 214-1).

¹H-NMR (CDCl₃) δ:0.99 (6H, d, J=6.6 Hz), 1.39 (9H, s), 2.17-2.30 (1H, m), 2.36 (3H, s), 2.62 (3H, s), 2.78 (2H, d, J=7.4 Hz), 3.51 (2H, s), 3.56 (3H, s), 4.10 (2H, d, J=4.7 Hz), 4.20 (1H, s), 4.61 (2H, s), 6.78 (2H, d, J=8.5 Hz), 7.06 (2H, d, J=8.5 Hz), 7.12-7.20 (4H, m).

2) Methyl (4-{[5-{[(tert-butoxycarbonyl)amino]methyl}-6-isobutyl-2-methyl-4-(4-methylphenyl)pyridin-3-yl]methoxy}phenyl)acetate (570 mg, 1.04 mmol) was dissolved in trifluoroacetic acid (10 mL) and the mixture was stirred for 1 hr. The reaction mixture was concentrated under reduced pressure and the residue was partitioned between ethyl acetate and saturated aqueous sodium hydrogen carbonate. The organic layer was dried over anhydrous magnesium sulfate and the solvent was evaporated under reduced pressure. The residue was purified by silica gel column chromatography to give methyl (4-{[5-(aminomethyl)-6-isobutyl-2-methyl-4-(4-methylphenyl)pyridin-3-yl]methoxy}phenyl)acetate (300 mg, yield 65%) as a colorless oil.

¹H-NMR (DMSO-d₆)δ:0.98 (6H, d, J=6.6 Hz), 2.18-2.25 (1H, m), 2.34 (3H, s), 2.60 (3H, s), 2.88 (2H, d, J=7.4 Hz), 3.30 (2H, d, J=5.3 Hz), 3.61 (3H, s), 4.20 (2H, d, J=4.7 Hz), 4.60 (2H, s),6.70 (2H, d, J=8.5 Hz), 6.79 (2H, d, J=8.5 Hz), 7.05 (2H, d, J=8.3 Hz), 7.15 (2H, d, J=8.3 Hz).

### Example 238

### 3-({[5-(aminomethyl)-6-isobutyl-2-methyl-4-(4-methylphenyl)pyridin-3-yl]amino}carbonyl)benzoic acid dihydrochloride

3-({[5-(Aminomethyl)-6-isobutyl-2-methyl-4-(4-methylphenyl)pyridin-3-yl]amino}carbonyl)benzoic acid dihydrochloride (230 mg, yield 89%) was obtained as a white powder from tert-butyl {[5-amino-2-isobutyl-6-methyl-4-(4-methylphenyl)pyridin-3-yl]methyl}carbamate (192 mg, 0.5 mmol) and isophthalic acid monomethyl ester chloride (149 mg, 0.75 mmol) according to a method similar to the method of Example 223.

¹H-NMR (DMSO-d₆) δ:1.01 (6H, d, J=6.6 Hz), 2.18-2.31 (1H, m), 2.31 (3H, s), 2.60 (3H, s), 3.04 (2H, brs), 3.85 (2H, brs), 7.25 (4H, s), 7.57 (1H, t, J=7.8 Hz), 7.86 (1H, d, J=7.8 Hz), 8.07 (1H, d, J=7.8 Hz), 8.16 (1H, s), 8.36 (3H, brs), 10.19 (1H, brs).

### Example 239

### methyl 3-{[5-(aminomethyl)-6-isobutyl-2-methyl-4-(4-methylphenyl)pyridin-3-yl]methoxy}-1H-indole-2-carboxylate

1) Methyl 3-{[5-{[(tert-butoxycarbonyl)amino]methyl}-6-isobutyl-2-methyl-4-(4-methylphenyl)pyridin-3-yl]methoxy}-1H-indole-2-carboxylate (0.41 g, yield 52%) was obtained as a pale-yellow solid from tert-butyl {[5-(hydroxymethyl)-2-isobutyl-6-methyl-4-(4-methylphenyl)pyridin-3-yl]methyl}carbamate (0.60 g, 1.49 mmol) and methyl 3-hydroxyindole-2-carboxylate (0.26 g, 1.36 mmol) according to a method similar to the method of Example 214-1).

¹H-NMR (CDCl₃) δ:0.97 (6H, d, J=6.8 Hz), 1.37 (9H, s), 2.17-2.26 (1H, m), 2.37 (3H, s), 2.77 (2H, d, J=7.2 Hz), 2.86 (3H, s), 3.82 (3H, s), 4.00 (2H, d, J=4.5 Hz), 4.09 (1H, brs), 5.03 (2H, s), 6.74-6.89 (4H, m), 7.09 (2H, d, J=7.9 Hz), 7.21-7.31 (2H, m), 8.28 (1H, brs).

2) Methyl 3-{[5-{[(tert-butoxycarbonyl)amino]methyl}-6-isobutyl-2-methyl-4-(4-methylphenyl)pyridin-3-yl]methoxy}-1H-indole-2-carboxylate (0.26 g, 1.36 mmol) was dissolved in 4N hydrogen chloride ethyl acetate solution (10 mL) and the mixture was stirred at room temperature for 30 min. The reaction mixture was neutralized with saturated aqueous sodium hydrogen carbonate and extracted with ethyl acetate. The extract was dried over anhydrous magnesium sulfate and the solvent was evaporated under reduced pressure. The obtained yellow solid was recrystallized from ethyl acetate-hexane to give methyl 3-{[5-(aminomethyl)-6-isobutyl-2-methyl-4-(4-methylphenyl)pyridin-3-yl]methoxy}-1H-indole-2-carboxylate (256 mg, yield 75%) as pale-yellow crystals.

¹H-NMR (CDCl₃) δ:0.98 (6H, d, J=6.6 Hz), 2.17-2.30 (1H, m), 2.38 (3H, s), 2.79 (2H, d, J=7.4 Hz), 2.86 (3H, s), 3.51 (2H, s), 3.83 (3H, s), 5.02 (2H, s), 6.77-6.88 (4H, m), 7.10 (2H, d, J=7.7 Hz), 7.22-7.28 (2H, m), 8.27 (1H, brs).

### Example 240

### 4-cyanobenzyl 5-(aminomethyl)-6-isobutyl-2-methyl-4-(4-methylphenyl)nicotinate

1) 4-Cyanobenzyl 5-{[(tert-butoxycarbonyl)amino]methyl}-6-isobutyl-2-methyl-4-(4-methylphenyl)nicotinate (2.32 g, yield 86%) was obtained as a yellow oil from 5-{[(tert-butoxycarbonyl)amino]methyl}-6-isobutyl-2-methyl-4-(4-methylphenyl)nicotinic acid (2.10 g, 5.10 mmol) and 4-cyanobenzyl bromide (1.00 g, 5.10 mmol) according to a method similar to the method of Example 169-1).

¹H-NMR (CDCl₃) δ:0.97 (6H, d, J=6.8 Hz), 1.38 (9H, s), 2.17-2.26 (1H, m), 2.37 (3H, s), 2.54 (3H, s), 2.78 (2H, d, J=7.2 Hz), 4.11-4.13 (2H, m), 4.20 (1H, brs), 4.98 (2H, s), 7.01 (2H, d, J=8.1 Hz), 7.10 (4H, d, J=8.1 Hz), 7.54 (2H, d, J=8.3 Hz).

2) 4-Cyanobenzyl 5-{[(tert-butoxycarbonyl)amino]methyl}-6-isobutyl-2-methyl-4-(4-methylphenyl)nicotinate (0.52 g, 0.985 mmol) was dissolved in trifluoroacetic acid (10 mL) and the mixture was stirred at room temperature for 1 hr. The reaction mixture was neutralized with saturated aqueous sodium hydrogen carbonate and extracted twice with ethyl acetate. The extract was dried over anhydrous magnesium sulfate to give 4-cyanobenzyl 5-(aminomethyl)-6-isobutyl-2-methyl-4-(4-methylphenyl)nicotinate (0.42 g, yield 99%) as a yellow oil.

¹H-NMR (CDCl₃) δ:0.90 (6H, d, J=6.6 Hz), 2.08-2.17 (1H, m), 2.32 (3H, s), 2.54 (3H, s), 2.70 (2H, d, J=7.0 Hz), 3.97 (2H, s), 4.99 (2H, s), 7.00 (2H, d, J=8.1 Hz), 7.08-7.14 (4H, m), 7.54 (2H, d, J=8.3 Hz).

### Example 241

### N-[5-(aminomethyl)-6-isobutyl-2-methyl-4-(4-methylphenyl)pyridin-3-yl]quinoxaline-2-carboxamide dihydrochloride

N-[5-(Aminomethyl)-6-isobutyl-2-methyl-4-(4-methylphenyl)pyridin-3-yl]quinoxaline-2-carboxamide dihydrochloride (137 mg, yield 50%) was obtained as a white powder from tert-butyl {[5-amino-2-isobutyl-6-methyl-4-(4-methylphenyl)pyridin-3-yl]methyl}carbamate (192 mg, 0.5 mmol) and quinoxaline-2-carbonyl chloride (144 mg, 0.75 mmol) according to a method similar to the method of Example 223.

¹H-NMR (DMSO-d₆) δ:1.02 (6H, d, J=6.6 Hz), 2.22-2.29 (1H, m), 2.23 (3H, s), 2.64 (3H, s), 3.06 (2H, brs), 3.86 (2H, brs), 7.22 (2H, d, J=8.1 Hz), 7.29 (2H, d, J=8.1 Hz), 7.96-8.04 (2H, m), 8.11-8.28 (2H, m), 8.39 (3H, brs), 9.34 (1H, s), 10.50 (1H, brs).

### Example 242

### N-[5-(aminomethyl)-6-isobutyl-2-methyl-4-(4-methylphenyl)pyridin-3-yl]-2,5-dimethylfuran-3-carboxamide dihydrochloride

N-[5-(Aminomethyl)-6-isobutyl-2-methyl-4-(4-methylphenyl)pyridin-3-yl]-2,5-dimethylfuran-3-carboxamide dihydrochloride (215 mg, yield 90%) was obtained as a white powder from tert-butyl {[5-amino-2-isobutyl-6-methyl-4-(4-methylphenyl)pyridin-3-yl]methyl}carbamate (192 mg, 0.5 mmol) and 2,5-dimethylfuran-3-carbonyl chloride (119 mg, 0.75 mmol) according to a method similar to the method of Example 223.

¹H-NMR (DMSO-d₆) δ:0.99 (6H, d, J=6.6 Hz), 2.17 (3H, s), 2.17-2.29 (1H, m), 2.29 (3H, s), 2.34 (3H, s), 2.54 (3H, s), 2.99 (2H, brs), 3.82 (2H, d, J=5.1 Hz), 6.25 (1H, s), 7.20 (2H, d, J=8.1 Hz), 7.26 (2H, d, J=8.1 Hz), 8.28 (3H, brs), 9.32 (1H, brs).

### Example 243

### N-[5-(aminomethyl)-6-isobutyl-2-methyl-4-(4-methylphenyl)pyridin-3-yl]-3-methylthiophene-2-carboxamide dihydrochloride

N-[5-(Aminomethyl)-6-isobutyl-2-methyl-4-(4-methylphenyl)pyridin-3-yl]-3-methylthiophene-2-carboxamide dihydrochloride (215 mg, yield 90%) was obtained as a white powder from tert-butyl {[5-amino-2-isobutyl-6-methyl-4-(4-methylphenyl)pyridin-3-yl]methyl}carbamate (192 mg, 0.5 mmol) and 3-methylthiophene-2-carbonyl chloride (120 mg, 0.75 mmol) according to a method similar to the method of Example 223.

¹H-NMR (DMSO-d₆) δ:0.98 (6H, d, J=6.6 Hz), 2.08 (3H, s), 2.09-2.33 (1H, m), 2.34 (3H, s), 2.51 (3H, s), 2.91 (2H, brs), 3.82 (2H, brs), 6.89 (1H, d, J=5.1 Hz), 7.19 (2H, d, J=7.8 Hz), 7.27 (2H, d, J=7.8 Hz), 7.55 (1H, d, J=5.1 Hz), 8.17 (3H, brs), 9.37 (1H, brs).

### Example 244

### N-[5-(aminomethyl)-6-isobutyl-2-methyl-4-(4-methylphenyl)pyridin-3-yl]-1-benzothiophene-2-carboxamide dihydrochloride

N-[5-(Aminomethyl)-6-isobutyl-2-methyl-4-(4-methylphenyl)pyridin-3-yl]-1-benzothiophene-2-carboxamide dihydrochloride (215 mg, yield 90%) was obtained as a white powder from tert-butyl {[5-amino-2-isobutyl-6-methyl-4-(4-methylphenyl)pyridin-3-yl]methyl}carbamate (192 mg, 0.5 mmol) and 1-benzothiophene-2-carbonyl chloride (150 mg, 0.75 mmol) according to a method similar to the method of Example 223.

¹H-NMR (DMSO-d₆) δ:1.00 (6H, d, J=6.6 Hz), 2.20-2.28 (1H, m), 2.28 (3H, s), 2.60 (3H, s), 3.00 (2H, brs), 3.84 (2H, d, J=5.4 Hz), 7.25 (4H, s), 7.41-7.50 (2H, m), 7.91 (1H, d, J=6.9 Hz), 8.00 (1H, d, J=6.9 Hz), 8.04 (1H, s), 8.33 (3H, brs), 10.34 (1H, brs).

### Example 245

### N-[5-(aminomethyl)-6-isobutyl-2-methyl-4-(4-methylphenyl)pyridin-3-yl]-3-methyl-1-benzofuran-2-carboxamide dihydrochloride

N-[5-(Aminomethyl)-6-isobutyl-2-methyl-4-(4-methylphenyl)pyridin-3-yl]-3-methyl-1-benzofuran-2-carboxamide dihydrochloride (213 mg, yield 90%) was obtained as a white powder from tert-butyl {[5-amino-2-isobutyl-6-methyl-4-(4-methylphenyl)pyridin-3-yl]methyl}carbamate (192 mg, 0.5 mmol) and 3-methyl-1-benzofuran-2-carbonyl chloride (150 mg, 0.75 mmol) according to a method similar to the method of Example 223.

¹H-NMR (DMSO-d₆) δ:1.00 (6H, d, J=6.6 Hz), 2.16-2.29 (1H, m), 2.29 (3H, s), 2.41 (3H, s), 2.60 (3H, s), 3.03 (2H, brs), 3.83 (2H, brs), 7.25 (4H, s), 7.35 (1H, t, J=6.9 Hz), 7.49 (1H, t, J=6.9 Hz), 7.56 (1H, d, J=6.9 Hz), 7.73 (1H, d, J=6.9 Hz), 8.35 (3H, brs), 10.08 (1H, brs).

### Example 246

### methyl [4-({[5-(aminomethyl)-6-isobutyl-2-methyl-4-(4-methylphenyl)pyridin-3-yl]amino}carbonyl)-2-oxopiperazin-1-yl]acetate dihydrochloride

1) Methyl [4-({[5-{[(tert-butoxycarbonyl)amino]methyl}-6-isobutyl-2-methyl-4-(4-methylphenyl)pyridin-3-yl]amino}carbonyl)-2-oxopiperazin-1-yl]acetate was obtained as an oil from 5-{[(tert-butoxycarbonyl)amino]methyl}-6-isobutyl-2-methyl-4-(4-methylphenyl)nicotinic acid (412 mg, 1.0 mmol) and methyl (2-oxopiperazin-1-yl)acetate (344 mg, 2.0 mmol) according to a method similar to the method of Example 95-1).

EIMS(M+1):582

2) Methyl [4-({[5-(aminomethyl)-6-isobutyl-2-methyl-4-(4-methylphenyl)pyridin-3-yl]amino}carbonyl)-2-oxopiperazin-1-yl]acetate dihydrochloride (271 mg, yield 49%) was obtained as a white powder from the oil obtained in the aforementioned 1), according to a method similar to the method of Example 2-3).

¹H-NMR (DMSO-d₆)δ:0.98 (6H, d, J=6.3 Hz), 1.99-2.28 (1H, m), 2.37 (3H, s), 2.50 (3H, s), 2.60 (2H, brs), 3.14 (2H, t, J=5.1 Hz), 3.46 (2H, t, J=5.1 Hz), 3.66 (3H, s), 3.81 (4H, brs), 4.08 (2H, s), 7.17 (2H, d, J=7.8 Hz), 7.29 (2H, d, J=7.8 Hz), 8.43 (3H, brs).

### Example 247

### [5-(methoxycarbonyl)pyridin-2-yl]methyl 5-(aminomethyl)-6-isobutyl-2-methyl-4-(4-methylphenyl)nicotinate

1) To a solution of 5-{[(tert-butoxycarbonyl)amino]methyl}-6-isobutyl-2-methyl-4-(4-methylphenyl)nicotinic acid (1.85 g, 4.48 mmol), methyl 6-(hydroxymethyl)nicotinate (0.68 g, 4.07 mmol) and triphenylphosphine (1.39 g, 5.29 mmol) in tetrahydrofuran (20 mL) was added 40% diethyl azodicarboxylate toluene solution (2.3 mL, 5.29 mmol) and the mixture was stirred at room temperature for 30 min. The solvent was evaporated under reduced pressure and the obtained residue was purified by silica gel column chromatography to give [5-(methoxycarbonyl)pyridin-2-yl]methyl 5-{[(tert-butoxycarbonyl)amino]methyl}-6-isobutyl-2-methyl-4-(4-methylphenyl)nicotinate (2.29 g, yield 99%) as a white solid.

¹H-NMR (CDCl₃) δ:0.97 (6H, d, J=6.6 Hz), 1.39 (9H, s), 2.17-2.26 (1H, m), 2.35 (3H, s), 2.58 (3H, s), 2.79 (2H, d, J=7.2 Hz), 3.96 (3H, s), 4.13-4.15 (2H, m), 4.21 (1H, brs), 5.11 (2H, s), 6.88 (1H, d, J=8.5 Hz), 7.06 (2H, d, J=8.1 Hz), 7.13 (2H, d, J=7.9 Hz), 8.14 (1H, dd, J=8.2, 2.2 Hz), 9.10 (1H, dd, J=2.1, 0.75 Hz).

2) [5-(Methoxycarbonyl)pyridin-2-yl]methyl 5-{[(tert-butoxycarbonyl)amino[methyl}-6-isobutyl-2-methyl-4-(4-methylphenyl)nicotinate (0.37 g, 0.659 mmol) was dissolved in 4N hydrogen chloride ethyl acetate solution (10 mL) and the mixture was stirred at room temperature for 30 min. The reaction mixture was neutralized with saturated aqueous sodium hydrogen carbonate and extracted with ethyl acetate. The extract was dried over anhydrous magnesium sulfate and the solvent was evaporated under reduced pressure to give [5-(methoxycarbonyl)pyridin-2-yl]methyl 5-(aminomethyl)-6-isobutyl-2-methyl-4-(4-methylphenyl)nicotinate (142 mg, yield 46%) as a colorless oil.

¹H-NMR (CDCl₃) δ:0.98 (6H, d, J=6.6 Hz), 2.17-2.29 (1H, m), 2.35 (3H, s), 2.57 (3H, s), 2.81 (2H, d, J=7.4 Hz), 3.65 (2H, s), 3.96 (3H, s), 5.11 (2H, s), 6.89 (1H, d, J=8.3 Hz), 7.10-7.16 (4H, m), 8.14 (1H, dd, J=8.2, 2.2 Hz), 9.10 (1H, d, J=1.3 Hz).

### Example 248

### 6-[({[5-(aminomethyl)-6-isobutyl-2-methyl-4-(4-methylphenyl)pyridin-3-yl]carbonyl}oxy)methyl]nicotinic acid trihydrochloride

1) 6-[({[5-{[(tert-Butoxycarbonyl)amino]methyl}-6-isobutyl-2-methyl-4-(4-methylphenyl)pyridin-3-yl]carbonyl}oxy)methyl]nicotinic acid (1.08 g, yield 58%) was obtained as a colorless oil from [5-(methoxycarbonyl)pyridin-2-yl]methyl 5-{[(tert-butoxycarbonyl)amino]methyl}-6-isobutyl-2-methyl-4-(4-methylphenyl)nicotinate (1.90 g, 3.38 mmol) according to a method similar to the method of Example 9-1).

¹H-NMR (CDCl₃) δ:0.98 (6H, d, J=6.8 Hz), 1.39 (9H, s), 2.27-2.35 (4H, m), 2.60 (3H, s), 2.81 (2H, d, J=7.2 Hz), 4.14-4.15 (2H, m), 4.25 (1H, brs), 5.14 (2H, s), 6.88-6.95 (1H, m), 7.06-7.19 (4H, m), 8.19 (1H, dd, J=8.2, 2.2 Hz), 9.16 (1H, s).

2) 6-[({[5-(Aminomethyl)-6-isobutyl-2-methyl-4-(4-methylphenyl)pyridin-3-yl]carbonyl}oxy)methyl]nicotinic acid trihydrochloride (413 mg, yield 81%) was obtained as a white solid from 6-[({[5-{[(tert-butoxycarbonyl)amino]methyl}-6-isobutyl-2-methyl-4-(4-methylphenyl)pyridin-3-yl]carbonyl}oxy)methyl]nicotinic acid (0.50 g, 0.913 mmol) according to a method similar to the method of Example 2-3).

¹H-NMR (DMSO-d₆) δ:0.97 (6H, d, J=6.6 Hz), 2.18-2.28 (1H, m), 2.33 (3H, s), 2.63 (3H, brs), 2.90-2.97 (2H, m), 3.82 (2H, d, J=5.1 Hz), 5.15 (2H, s), 7.03 (1H, d, J=8.1 Hz), 7.17-7.23 (4H, m), 8.17 (1H, dd, J=8.2, 2.0 Hz), 8.38 (3H, brs), 8.98 (1H, d, J=1.5 Hz).

### Example 249

### (5-(aminocarbonyl)pyridin-2-yl]methyl 5-(aminomethyl)-6-isobutyl-2-methyl-4-(4-methylphenyl)nicotinate

1) [5-(Aminocarbonyl)pyridin-2-yl]methyl 5-{[(tert-butoxycarbonyl)amino]methyl}-6-isobutyl-2-methyl-4-(4-methylphenyl)nicotinate (222 mg, yield 38%) was obtained as a colorless oil from 6-[({[5-{[(tert-butoxycarbonyl)amino]methyl}-6-isobutyl-2-methyl-4-(4-methylphenyl)pyridin-3-yl]carbonyl}oxy)methyl]nicotinic acid (0.58 g, 1.06 mmol) according to a method similar to the method of Example 3-1).

¹H-NMR (CDCl₃) δ:0.97 (6H, d, J=6.6 Hz), 1.39 (9H, s), 2.17-2.26 (1H, m), 2.36 (3H, s), 2.58 (3H, s), 2.79 (2H, d, J=7.4 Hz), 4.13-4.15 (2H, m), 4.22 (1H, brs), 5.10 (2H, s), 6.92 (1H, d, J=7.9 Hz), 7.07 (2H, d, J=8.1 Hz), 7.14 (2H, d, J=7.9 Hz), 8.03 (1H, dd, J=8.3, 2.3 Hz), 8.89 (1H, d, J=2.3 Hz).

2) [5-(Aminocarbonyl)pyridin-2-yl]methyl 5-(aminomethyl)-6-isobutyl-2-methyl-4-(4-methylphenyl)nicotinate (159 mg, yield 87%) was obtained as a colorless oil from [5-(aminocarbonyl)pyridin-2-yl]methyl 5-{[(tert-butoxycarbonyl)amino]methyl}-6-isobutyl-2-methyl-4-(4-methylphenyl)nicotinate (0.22 g, 0.406 mmol) according to a method similar to the method of Example 247-2).

¹H-NMR (CDCl₃) δ:0.98 (6H, d, J=6.6 Hz), 2.15-2.31 (1H, m), 2.36 (3H, s), 2.57 (3H, s), 2.81 (2H, d, J=7.4 Hz), 3.65 (2H, s), 5.10 (2H, s), 6.94 (1H, d, J=7.7 Hz), 7.11-7.17 (4H, m), 8.03 (1H, dd, J=8.1, 2.3 Hz), 8.89 (1H, d, J=2.3 Hz).

### Example 250

### ethyl 4-{[5-(aminomethyl)-6-isobutyl-2-methyl-4-(4-methylphenyl)pyridin-3-yl]methoxy}-2-ethylpyrimidine-5-carboxylate tetrahydrochloride

1) Ethyl 4-{[5-{[(tert-butoxycarbonyl)amino]methyl}-6-isobutyl-2-methyl-4-(4-methylphenyl)pyridin-3-yl]methoxy}-2-ethylpyrimidine-5-carboxylate (308 mg, yield 40%) was obtained as a white solid from tert-butyl {[5-(hydroxymethyl)-2-isobutyl-6-methyl-4-(4-methylphenyl)pyridin-3-yl]methyl}carbamate (0.53 g, 1.33 mmol) and ethyl 2-ethyl-4-hydroxypyrimidine-5-carboxylate (0.26 g, 1.33 mmol) according to a method similar to the method of Example 214-1).

¹H-NMR (CDCl₃) δ:0.99 (6H, d, J=6.8 Hz), 1.20-1.29 (6H, m), 1.39 (9H, s), 2.19-2.28 (1H, m), 2.34 (3H, s), 2.67 (3H, s), 2.75-2.83 (4H, m), 4.10 (2H, d, J=4.9 Hz), 4.27-4.34 (3H, m), 5.22 (2H, s), 7.06 (2H, d, J=8.1 Hz), 7.14 (2H, d, J=7.9 Hz), 8.86 (1H, s).

2) Ethyl 4-{[5-(aminomethyl)-6-isobutyl-2-methyl-4-(4-methylphenyl)pyridin-3-yl]methoxy}-2-ethylpyrimidine-5-carboxylate tetrahydrochloride (269 mg, yield 80%) was obtained as a white solid from ethyl 4-{[5-{[(tert-butoxycarbonyl)amino]methyl}-6-isobutyl-2-methyl-4-(4-methylphenyl)pyridin-3-yl]methoxy}-2-ethylpyrimidine-5-carboxylate (308 mg, 0.536 mmol) according to a method similar to the method of Example 2-3).

¹H-NMR (DMSO-d₆) δ:0.98 (6H, d, J=6.6 Hz), 1.19 (3H, t, J=7.5 Hz), 1.25 (3H, t, J=7.1 Hz), 2.14-2.23 (1H, m), 2.43 (3H, s), 2.58-2.67 (2H, m), 2.81-2.97 (3H, m), 3.13 (2H, brs), 3.73-3.83 (2H, m), 4.22 (2H, t, J=7.0 Hz), 4.42 (2H, s), 7.25-7.31 (2H, m), 7.38-7.43 (2H, m), 8.43 (3H, brs), 8.46 (1H, s).

### Example 251

### 4-(1H-tetrazol-5-yl)benzyl 5-(aminomethyl)-6-isobutyl-2-methyl-4-(4-methylphenyl)nicotinate dihydrochloride

1) A solution of 4-cyanobenzyl 5-{[(tert-butoxycarbonyl)amino]methyl}-6-isobutyl-2-methyl-4-(4-methylphenyl)nicotinate (1.28 g, 2.43 mmol) and tributyltin azide (2.3 mL, 8.49 mmol) in toluene (7.5 mL) was heated under reflux under an argon atmosphere for 3 hrs. The solvent was evaporated under reduced pressure and the obtained residue was purified by silica gel column chromatography to give 4-(1H-tetrazol-5-yl)benzyl 5-{[(tert-butoxycarbonyl)amino]methyl}-6-isobutyl-2-methyl-4-(4-methylphenyl)nicotinate (1.23 g, yield 88%) as a colorless oil.

¹H-NMR (CDCl₃) δ:0.96 (6H, d, J=6.6 Hz), 1.39 (9H, s), 2.15-2.24 (1H, m), 2.25 (3H, s), 2.54 (3H, s), 2.83 (2H, d, J=7.2 Hz), 4.18 (2H, d, J=4.9 Hz), 4.32 (1H, brs), 5.00 (2H, s), 7.01 (2H, d, J=7.9 Hz), 7.07 (2H, d, J=7.9 Hz), 7.18 (2H, d, J=8.1 Hz), 8.03 (2H, d, J=8.1 Hz).

2) 4-(1H-Tetrazol-5-yl)benzyl 5-(aminomethyl)-6-isobutyl-2-methyl-4-(4-methylphenyl)nicotinate dihydrochloride (688 mg, yield 95%) was obtained as a white solid from 4-(1H-tetrazol-5-yl)benzyl 5-{[(tert-butoxycarbonyl)amino]methyl}-6-isobutyl-2-methyl-4-(4-methylphenyl)nicotinate (0.75 g, 1.33 mmol) according to a method similar to the method of Example 2-3).

¹H-NMR (DMSO-d₆) δ:0.96 (6H, d, J=6.6 Hz), 2.17-2.26 (1H, m), 2.30 (3H, s), 2.54 (3H, s), 2.87 (2H, d, J=6.8 Hz), 3.81 (2H, 30 d, J=5.5 Hz), 5.08 (2H, s), 7.14-7.25 (6H, m), 8.02 (2H, d, J=8.1 Hz), 8.22 (3H, brs).

### Example 252

### 5-[({[5-(aminomethyl)-6-isobutyl-2-methyl-4-(4-methylphenyl)pyridin-3- yl]carbonyl}oxy)methyl]furan-2-carboxylic acid dihydrochloride

1) [5-(Methoxycarbonyl)-2-furyl]methyl 5-{[(tert-butoxycarbonyl)amino]methyl}-6-isobutyl-2-methyl-4-(4-methylphenyl)nicotinate (2.37 g, yield 88%) was obtained as a yellow oil from 5-{[(tert-butoxycarbonyl)amino]methyl}-6-isobutyl-2-methyl-4-(4-methylphenyl)nicotinic acid (2.00 g, 4.85 mmol) and methyl 5-(chloromethyl)furan-2-carboxylate (0.85 g, 4.85 mmol) according to a method similar to the method of Example 169-1).

¹H-NMR (CDCl₃) δ:0.96 (6H, d, J=6.6 Hz), 1.38 (9H, s), 2.13-2.24 (1H, m), 2.35 (3H, s), 2.52 (3H, s), 2.77 (2H, d, J=7.2 Hz), 3.91 (3H, s), 4.11 (2H, d, J=5.1 Hz), 4.19 (1H, brs), 4.94 (2H, s), 6.24 (1H, d, J=3.6 Hz), 7.00 (2H, d, J=8.1 Hz), 7.06 (1H, d, J=3.6 Hz), 7.11 (2H, d, J=7.9 Hz).

2) 5-[({[5-{[(tert-Butoxycarbonyl)amino]methyl}-6-isobutyl-2-methyl-4-(4-methylphenyl)pyridin-3-yl]carbonyl}oxy)methyl]furan-2-carboxylic acid (1.95 g, yield 95%) was obtained as a white solid from [5-(methoxycarbonyl)-2-furyl]methyl 5-{[(tert-butoxycarbonyl)amino]methyl}-6-isobutyl-2-methyl-4-(4-methylphenyl)nicotinate (2.11 g, 3.83 mmol) according to a method similar to the method of Example 9-1).

¹H-NMR (CDCl₃) δ:0.96 (6H, d, J=6.6 Hz), 1.38 (9H, s), 2.14-2.25 (1H, m), 2.36 (3H, s), 2.53 (3H, s), 2.86 (2H, d, J=7.0 Hz), 4.09-4.18 (2H, m), 4.26 (1H, brs), 4.99 (2H, s), 6.32 (1H, d, J=3.4 Hz), 7.03 (2H, d, J=8.1 Hz), 7.10-7.18 (3H, m).

3) 5-[({[5-(Aminomethyl)-6-isobutyl-2-methyl-4-(4-methylphenyl)pyridin-3-yl]carbonyl}oxy)methyl]furan-2-carboxylic acid dihydrochloride (460 mg, yield 79%) was obtained as a white solid from 5-[({[5-{[(tert-butoxycarbonyl)amino]methyl}-6-isobutyl-2-methyl-4-(4-methylphenyl)pyridin-3-yl]carbonyl}oxy)methyl]furan-2-carboxylic acid (0.61 g, 1.14 mmol) according to a method similar to the method of Example 2-3).

¹H-NMR (DMSO-d₆) δ:0.96 (6H, d, J=6.6 Hz), 2.16-2.27 (1H, m), 2.33 (3H, s), 2.90 (2H, brs), 3.80 (2H, d, J=5.3 Hz), 5.05 (2H, s), 6.46 (1H, d, J=3.4 Hz), 7.11-7.14 (3H, m), 7.17 (2H, d, J=8.1 Hz), 8.29 (3H, brs).

### Example 253

### [5-(aminocarbonyl)-2-furyl]methyl 5-(aminomethyl)-6-isobutyl-2-methyl-4-(4-methylphenyl)nicotinate dihydrochloride

1) [5-(Aminocarbonyl)-2-furyl]methyl 5-{[(tert-butoxycarbonyl)amino]methyl}-6-isobutyl-2-methyl-4-(4-methylphenyl)nicotinate (520 mg, yield 69%) was obtained as a colorless oil from 5-[({[5-{[(tert-butoxycarbonyl)amino]methyl}-6-isobutyl-2-methyl-4-(4-methylphenyl)pyridin-3-yl]carbonyl}oxy)methyl]-furan-2-carboxylic acid (0.75 g, 1.40 mmol) according to a method similar to the method of Example 3-1).

¹H-NMR (CDCl₃) δ:0.96 (6H, d, J=6.6 Hz), 1.38 (9H, s), 2.14-2.27 (1H, m), 2.35 (3H, s), 2.52 (3H, s), 2.78 (2H, d, J=7.4 Hz), 4.06-4.13 (2H, m), 4.19 (1H, brs), 4.94 (2H, s), 5.45 (1H, brs), 6.16 (1H, brs), 6.27 (1H, d, J=3.4 Hz), 6.98 (2H, d, J=8.1 Hz), 7.04 (1H, d, J=3.6 Hz), 7.09 (2H, d, J=7.9 Hz).

2) [5-(Aminocarbonyl)-2-furyl]methyl 5-(aminomethyl)-6-isobutyl-2-methyl-4-(4-methylphenyl)nicotinate dihydrochloride (471 mg, yield 95%) was obtained as a white solid from [5-(aminocarbonyl)-2-furyl]methyl 5-{[(tert-butoxycarbonyl)amino]methyl}-6-isobutyl-2-methyl-4-(4-methylphenyl)nicotinate (0.52 g, 0.971 mmol) according to a method similar to the method of Example 2-3).

¹H-NMR (DMSO-d₆) δ:0.96 (6H, d, J=6.6 Hz), 2.14-2.27 (1H, m), 2.34 (3H, s), 2.88 (2H, brs), 3.80 (2H, d, J=5.5 Hz), 5.02 (2H, s), 6.39 (2H, d, J=3.4 Hz), 7.06 (1H, d, J=3.4 Hz), 7.12 (2H, d, J=7.9 Hz), 7.18 (2H, d, J=8.3 Hz), 7.43 (1H, brs), 7.73 (1H, brs), 8.28 (3H, brs).

### Example 254

### methyl 3-{[[5-(aminomethyl)-6-isobutyl-2-methyl-4-(4-methylphenyl)pyridin-3-yl](methyl)amino]carbonyl}benzoate dihydrochloride

To a mixture of 3-({[5-{[(tert-butoxycarbonyl)amino]methyl}-6-isobutyl-2-methyl-4-(4-methylphenyl)pyridin-3-yl]amino}carbonyl)benzoic acid (212 mg, 0.4 mmol), potassium carbonate (138 mg, 1.0 mmol) and N,N-dimethylformamide (5 mL) was added methyl iodide (282 mg, 2.0 mmol) and the mixture was stirred at room temperature for 8 hrs. Water was added to the reaction mixture and the mixture was extracted with ethyl acetate. The organic layer was washed with saturated brine and dried over anhydrous magnesium sulfate. The solvent was evaporated under reduced pressure and the obtained residue was purified by silica gel column chromatography to give an oil. To a solution of the obtained oil in ethyl acetate (1 mL) was added a 4N hydrogen chloride ethyl acetate solution (1 mL) and the mixture was stirred at room temperature for 1 hr. The solvent was evaporated under reduced pressure and the obtained residue was crystallized from hexane to give methyl 3-{[[5-(aminomethyl)-6-isobutyl-2-methyl-4-(4-methylphenyl)pyridin-3-yl](methyl)amino]carbonyl}benzoate dihydrochloride (203 mg, yield 95%) as a white powder.

EIMS(M+1):460

### Example 255

### N-[5-(aminomethyl)-6-isobutyl-2-methyl-4-(4-methylphenyl)pyridin-3-yl]isophthalamide dihydrochloride

1) tert-Butyl {[5-{[3-(aminocarbonyl)benzoyl]amino}-2-isobutyl-6-methyl-4-(4-methylphenyl)pyridin-3-yl]methyl}carbamate (248 mg, yield 98%) was obtained as a white powder from 3-({[5-{[(tert-butoxycarbonyl)amino]methyl}-6-isobutyl-2-methyl-4-(4-methylphenyl)pyridin-3-yl]amino}carbonyl)benzoic acid (260 mg, 0.48 mmol) according to a method similar to the method of Example 3-1).

¹H-NMR (CDCl₃) δ:0.99 (6H, d, J=6.6 Hz), 1.38 (9H, s), 2.20-2.31 (1H, m), 2.33 (3H, s), 2.49 (3H, s), 2.78 (2H, brs), 4.13 (2H, brs), 4.40 (1H, brs), 5.79 (1H, brs), 6.38 (1H, brs), 7.03 (2H, d, J=8.1 Hz), 7.18 (2H, d, J=8.1 Hz), 7.7.39-7.45 (1H, brs), 7.60-7.63 (1H, m), 7.88-7.92 (2H, m).

2) N-[5-(Aminomethyl)-6-isobutyl-2-methyl-4-(4-methylphenyl)pyridin-3-yl]isophthalamide dihydrochloride (233 mg, yield 99%) was obtained as a white powder from tert-butyl {[5-{[3-(aminocarbonyl)benzoyl]amino}-2-isobutyl-6-methyl-4-(4-methylphenyl)pyridin-3-yl]methyl}carbamate (248 mg, 0.47 mmol) according to a method similar to the method of Example 2-3).

¹H-NMR (DMSO-d₆) δ:1.00 (6H, d, J=6.3 Hz), 2.22-2.30 (1H, m), 2.30 (3H, s), 2.51 (3H, s), 2.89 (2H, brs), 3.84 (2H, brs), 7.23 (4H, s), 7.56 (1H, t, J=7.8 Hz), 7.83 (2H, d, J=7.8 Hz), 8.06 (2H, d, J=7.8 Hz), 8.14 (1H, s), 8.16 (3H, brs), 10.04 (1H, brs).

### Example 256

### 4-[2-oxo-2-(2-oxo-2-phenylethoxy)ethyl]benzyl 5-(aminomethyl)-6-isobutyl-2-methyl-4-(4-methylphenyl)nicotinate dihydrochloride

1) 4-[2-Oxo-2-(2-oxo-2-phenylethoxy)ethyl]benzyl 5-{[(tert-butoxycarbonyl)amino]methyl}-6-isobutyl-2-methyl-4-(4-methylphenyl)nicotinate (2.85 g, yield 86%) was obtained as a colorless oil from 5-{[(tert-butoxycarbonyl)amino]methyl}-6-isobutyl-2-methyl-4-(4-methylphenyl)nicotinic acid (2.00 g, 4.85 mmol) and phenacyl 4-(bromomethyl)phenylacetate (1.69 g, 4.85 mmol) according to a method similar to the method of Example 169-1).

¹H-NMR (CDCl₃) δ:0.96 (6H, d, J=6.6 Hz), 1.38 (9H, s), 2.15-2.24 (1H, m), 2.38 (3H, s), 2.52 (3H, s), 2.77 (2H, d, J=7.4 Hz), 3.82 (2H, s), 4.11-4.16 (2H, m), 4.21 (1H, brs), 4.91 (2H, s), 5.36 (2H, s), 7.02-7.05 (4H, m), 7.15 (2H, d, J=7.7 Hz), 7.26-7.29 (2H, m), 7.46-7.51 (2H, m), 7.58-7.64 (1H, m), 7.88-7.91 (2H, m).

2) 4-[2-Oxo-2-(2-oxo-2-phenylethoxy)ethyl]benzyl 5-(aminomethyl)-6-isobutyl-2-methyl-4-(4-methylphenyl)nicotinate dihydrochloride (117 mg, yield 45%) was obtained as a white solid from 4-[2-oxo-2-(2-oxo-2-phenylethoxy)ethyl]benzyl 5-{[(tert-butoxycarbonyl)amino]methyl}-6-isobutyl-2-methyl-4-(4-methylphenyl)nicotinate (0.27 g, 0.398 mmol) according to a method similar to the method of Example 2-3).

¹H-NMR (DMSO-d₆) δ:0.96 (6H, d, J=6.6 Hz), 2.16-2.27 (1H, m), 2.38 (3H, s), 2.83 (2H, brs), 3.81 (2H, d, J=5.3 Hz), 3.85 (2H, s), 4.95 (2H, s), 5.53 (2H, s), 7.02 (2H, d, J=8.1 Hz), 7.15 (2H, d, J=7.5 Hz), 7.26 (4H, t, J=7.72), 7.56 (2H, d, J=7.9 Hz), 7.67-7.72 (1H, m), 7.92-7.98 (2H, m), 8.17 (3H, brs).

### Example 257

### 4-(2-methoxy-2-oxoethyl)benzyl 5-(aminomethyl)-6-isobutyl-2-methyl-4-(4-methylphenyl)nicotinate dihydrochloride

1) {4-[({[5-{[(tert-Butoxycarbonyl)amino]methyl}-6-isobutyl-2-methyl-4-(4-methylphenyl)pyridin-3-yl]carbonyl}oxy)methyl]phenyl}acetic acid (1.65 g, yield 77%) was obtained as a colorless oil from 4-[2-oxo-2-(2-oxo-2-phenylethoxy)ethyl]benzyl 5-{[(tert-butoxycarbonyl)amino]methyl}-6-isobutyl-2-methyl-4-(4-methylphenyl)nicotinate (2.58 g, 3.80 mmol) according to a method similar to the method of Example 9-1).

¹H-NMR (CDCl₃) δ:0.95 (6H, d, J=6.6 Hz), 1.38 (9H, s), 2.14-2.23 (1H, m), 2.37 (3H, s), 2.52 (3H, s), 2.77 (2H, d, J=7.2 Hz), 3.65 (2H, s), 4.09-4.16 (2H, m), 4.21 (1H, brs), 4.90 (2H, s), 7.00-7.06 (4H, m), 7.13 (2H, d, J=7.9 Hz), 7.21 (2H, d, J=8.1 Hz).

2) To a mixture of {4-[({[5-{[(tert-butoxycarbonyl)amino]methyl}-6-isobutyl-2-methyl-4-(4-methylphenyl)pyridin-3-yl]carbonyl}oxy)methyl]phenyl}acetic acid (0.65 g, 1.16 mmol), potassium carbonate (0.32 g, 2.32 mmol) and N,N-dimethylformamide (15 mL) was added methyl iodide (197 mg, 1.39 mmol) and the mixture was stirred at room temperature for 1 hr. The reaction mixture was diluted with ethyl acetate, washed with saturated brine and dried over anhydrous magnesium sulfate. The solvent was evaporated under reduced pressure and the obtained residue was purified by silica gel column chromatography to give 4-(2-methoxy-2-oxoethyl)benzyl 5-{[(tert-butoxycarbonyl)amino]methyl}-6-isobutyl-2-methyl-4-(4-methylphenyl)nicotinate (0.56 g, yield 84%) as a colorless oil.

¹H-NMR (CDCl₃) δ:0.96 (6H, d, J=6.6 Hz), 1.38 (9H, s), 2.13-2.26 (1H, m), 2.38 (3H, s), 2.52 (3H, s), 2.77 (2H, d, J=7.4 Hz), 3.62 (2H, s), 3.70 (3H, s), 4.12-4.13 (2H, m), 4.20 (1H, brs), 4.90 (2H, s), 7.01-7.04 (4H, m), 7.14 (2H, d, J=7.9 Hz), 7.20 (2H, d, J=8.1 Hz).

3) 4-(2-Methoxy-2-oxoethyl)benzyl 5-(aminomethyl)-6-isobutyl-2-methyl-4-(4-methylphenyl)nicotinate dihydrochloride (483 mg, yield 90%) was obtained as a white solid from 4-(2-methoxy-2-oxoethyl)benzyl 5-{[(tert-butoxycarbonyl)amino]methyl}-6-isobutyl-2-methyl-4-(4-methylphenyl)nicotinate (0.56 g, 0.974 mmol) according to a method similar to the method of Example 2-3).

¹H-NMR (DMSO-d₆) δ:0.95 (6H, d, J=6.6 Hz), 2.14-2.26 (1H, m), 2.37 (3H, s), 2.79-2.88 (2H, m), 3.62 (3H, s), 3.69 (2H, s), 3.81 (2H, d, J=5.3 Hz), 4.94 (2H, s), 7.00 (2H, d, J=8.1 Hz), 7.13-7.24 (6H, m), 8.21 (3H, brs).

### Example 258

### {4-[({[5-(aminomethyl)-6-isobutyl-2-methyl-4-(4-methylphenyl)pyridin-3-yl]carbonyl}oxy)methyl]phenyl}acetic acid dihydrochloride

{4-[({[5-(Aminomethyl)-6-isobutyl-2-methyl-4-(4-methylphenyl)pyridin-3-yl]carbonyl}oxy)methyl]phenyl}acetic acid dihydrochloride (348 mg, yield 73%) was obtained as a white solid from {4-[({[5-{[(tert-butoxycarbonyl)amino]methyl}-6-isobutyl-2-methyl-4-(4-methylphenyl)pyridin-3-yl]carbonyl}oxy)methyl]phenyl}acetic acid (0.50 g, 0.892 mmol) according to a method similar to the method of Example 2-3).

¹H-NMR (DMSO-d₆) δ:0.96 (6H, d, J=6.6 Hz), 2.16-2.27 (1H, m), 2.37 (3H, s), 2.53 (3H, s), 2.90 (2H, d, J=5.8 Hz), 3.57 (2H, s), 3.82 (2H, d, J=5.3 Hz), 4.95 (2H, s), 6.99 (2H, d, J=8.1 Hz), 7.15 (2H, d, J=8.1 Hz), 7.20 (2H, d, J=8.1 Hz), 7.23 (2H, d, J=8.1 Hz), 8.30 (3H, brs).

### Example 259

### 4-(2-amino-2-oxoethyl)benzyl 5-(aminomethyl)-6-isobutyl-2-methyl-4-(4-methylphenyl)nicotinate dihydrochloride

1) 4-(2-Amino-2-oxoethyl)benzyl 5-{[(tert-butoxycarbonyl)amino]methyl}-6-isobutyl-2-methyl-4-(4-methylphenyl)nicotinate (360 mg, yield 72%) was obtained as a colorless oil from {4-[({[5-{[(tert-butoxycarbonyl)amino]methyl}-6-isobutyl-2-methyl-4-(4-methylphenyl)pyridin-3-yl]carbonyl}oxy)methyl]phenyl}acetic acid (0.50 g, 0.892 mmol) according to a method similar to the method of Example 3-1).

¹H-NMR (CDCl₃) δ:0.95 (6H, d, J=6.6 Hz), 1.38 (9H, s), 2.13-2.26 (1H, m), 2.39 (3H, s), 2.52 (3H, s), 2.77 (2H, d, J=7.4 Hz), 3.58 (2H, s), 4.12-4.13 (2H, m), 4.21 (1H, brs), 4.91 (2H, s), 5.31 (2H, brs), 7.04-7.06 (4H, m), 7.16 (2H, d, J=7.9 Hz), 7.20 (2H, d, J=8.1 Hz).

2) 4-(2-Amino-2-oxoethyl)benzyl 5-(aminomethyl)-6-isobutyl-2-methyl-4-(4-methylphenyl)nicotinate dihydrochloride (231 mg, yield 67%) was obtained as a white solid from 4-(2-amino-2-oxoethyl)benzyl 5-{[(tert-butoxycarbonyl)amino]methyl}-6-isobutyl-2-methyl-4-(4-methylphenyl)nicotinate (0.36 g, 0.643 mmol) according to a method similar to the method of Example 2-3).

¹H-NMR (DMSO-d₆) δ:0.95 (6H, d, J=6.6 Hz), 2.14-2.25 (1H, m), 2.38 (3H, s), 2.86 (2H, brs), 3.37 (2H, s), 3.81 (2H, d, J=5.5 Hz), 4.93 (2H, s), 6.88 (1H, brs), 6.98 (2H, d, J=8.1 Hz), 7.13-7.25 (6H, m), 7.49 (1H, brs), 8.21 (3H, brs).

### Example 260

### 4-(methylsulfonyl)benzyl 5-(aminomethyl)-6-isobutyl-2-methyl-4-(4-methylphenyl)nicotinate dihydrochloride

1) 4-(Methylsulfonyl)benzyl 5-{[(tert-butoxycarbonyl)amino]methyl}-6-isobutyl-2-methyl-4-(4-methylphenyl)nicotinate (530 mg, yield 73%) was obtained as a colorless oil from 5-{[(tert-butoxycarbonyl)amino]methyl}-6-isobutyl-2-methyl-4-(4-methylphenyl)nicotinic acid (495 mg, 1.20 mmol) and 1-(bromomethyl)-4-(methylsulfonyl)benzene (300 mg, 1.20 mmol) according to a method similar to the method of Example 169-1).

¹H-NMR (CDCl₃) δ:0.97 (6H, d, J=6.6 Hz), 1.39 (9H, s), 2.19-2.28 (1H, m), 2.38 (3H, s), 2.55 (3H, s), 2.78 (2H, d, J=7.4 Hz), 3.04 (3H, s), 4.12-4.13 (2H, m), 4.21 (1H, brs), 5.01 (2H, s), 7.04 (2H, d, J=8.1 Hz), 7.14 (2H, d, J=7.9 Hz), 7.19 (2H, d, J=8.3 Hz), 7.83 (2H, d, J=8.5 Hz).

2) 4-(Methylsulfonyl)benzyl 5-(aminomethyl)-6-isobutyl-2-methyl-4-(4-methylphenyl)nicotinate dihydrochloride (466 mg, yield 92%) was obtained as a white solid from 4-(methylsulfonyl)benzyl 5-{[(tert-butoxycarbonyl)amino]methyl}-6-isobutyl-2-methyl-4-(4-methylphenyl)nicotinate (0.53 g, 0.913 mmol) according to a method similar to the method of Example 2-3).

¹H-NMR (DMSO-d₆) δ:0.96 (6H, d, J=6.6 Hz), 2.15-2.26 (1H, m), 2.36 (3H, s), 2.54-2.58 (3H, m), 2.87-2.97 (2H, m), 3.22 (3H, s), 3.81 (2H, d, J=5.1 Hz), 5.11 (2H, s), 7.15-7.28 (6H, m), 7.84 (2H, d, J=8.3 Hz), 8.23-8.40 (3H, m).

### Example 261

### ethyl 3-[4-({[5-(aminomethyl)-6-isobutyl-2-methyl-4-(4-methylphenyl)pyridin-3-yl]amino}carbonyl)-2-oxopiperazin-1-yl]propionate dihydrochloride

1) Ethyl 3-[4-({[5-{[(tert-butoxycarbonyl)amino]methyl}-6-isobutyl-2-methyl-4-(4-methylphenyl)pyridin-3-yl]amino}carbonyl)-2-oxopiperazin-1-yl]propionate was obtained as an oil from 5-{[(tert-butoxycarbonyl)amino]methyl}-6-isobutyl-2-methyl-4-(4-methylphenyl)nicotinic acid (412 mg, 1.0 mmol) and ethyl (2-oxopiperazin-1-yl)propionate (250 mg, 2.0 mmol) according to a method similar to the method of Example 95-1).

EIMS(M+1):610

2) Ethyl 3-[4-({[5-(aminomethyl)-6-isobutyl-2-methyl-4-(4-methylphenyl)pyridin-3-yl]amino}carbonyl)-2-oxopiperazin-1-yl]propionate dihydrochloride (278 mg, yield 49%) was obtained as a white powder from the oil obtained in aforementioned 1), according to a method similar to the method of Example 2-3).

¹H-NMR (DMSO-d₆)δ:0.99 (6H, d, J=6.3 Hz), 1.19 (3H, t, J=7.2 Hz), 2.14-2.23 (1H, m), 2.37 (3H, s), 2.64 (2H, s), 3.06 (4H, brs), 3.37-3.47 (4H, m), 3.74 (2H, s), 3.83 (2H, brs), 4.06 (2H, q, J=7.2 Hz), 7.18 (2H, d, J=7.8 Hz), 7.29 (2H, d, J=7.8 Hz), 8.40 (3H, brs).

### Example 262

### N-[5-(aminomethyl)-6-isobutyl-2-methyl-4-(4-methylphenyl)pyridin-3-yl]-2-methoxybenzamide dihydrochloride

N-[5-(Aminomethyl)-6-isobutyl-2-methyl-4-(4-methylphenyl)pyridin-3-yl]-2-methoxybenzamide dihydrochloride (209 mg, yield 95%) was obtained as a white powder from tert-butyl {[5-amino-2-isobutyl-6-methyl-4-(4-methylphenyl)pyridin-3-yl]methyl}carbamate (192 mg, 0.5 mmol) and 2-methoxybenzoyl chloride (128 mg, 0.75 mmol) according to a method similar to the method of Example 223.

¹H-NMR (DMSO-d₆) δ:1.00 (6H, d, J=6.6 Hz), 2.18-2.29 (1H, m), 2.36 (3H, s), 2.61 (3H, s), 3.03 (2H, s), 3.69 (3H, s), 3.84 (2H, brs), 6.98 (1H, t, J=7.5 Hz), 7.08 (1H, d, J=8.1 Hz), 7.24 (2H, d, J=8.1 Hz), 7.32 (2H, d, J=8.1 Hz), 7.39-7.49(2H, m), 8.32 (3H, brs), 9.55 (1H, brs).

### Example 263

### N-[5-(aminomethyl)-6-isobutyl-2-methyl-4-(4-methylphenyl)pyridin-3-yl]-2-fluorobenzamide dihydrochloride

N-[5-(Aminomethyl)-6-isobutyl-2-methyl-4-(4-methylphenyl)pyridin-3-yl]-2-fluorobenzamide dihydrochloride (204 mg, yield 95%) was obtained as a white powder from tert-butyl {[5-amino-2-isobutyl-6-methyl-4-(4-methylphenyl)pyridin-3-yl]methyl}carbamate (192 mg, 0.5 mmol) and 2-fluorobenzoyl chloride (122 mg, 0.75 mmol) according to a method similar to the method of Example 223.

¹H-NMR (DMSO-d₆) δ:0.99 (6H, d, J=6.6 Hz), 2.21-2.28 (1H, m), 2.37 (3H, s), 2.55 (3H, s), 2.92 (2H, s), 3.84 (2H, s), 7.13-7.32 (7H, m), 7.49-7.54 (1H, m), 8.20 (3H, brs), 9.86 (1H, brs).

### Example 264

### N-[5-(aminomethyl)-6-isobutyl-2-methyl-4-(4-methylphenyl)pyridin-3-yl]-3-methoxybenzamide dihydrochloride

N-[5-(Aminomethyl)-6-isobutyl-2-methyl-4-(4-methylphenyl)pyridin-3-yl]-3-methoxybenzamide dihydrochloride (196 mg, yield 80%) was obtained as a white powder from tert-butyl {[5-amino-2-isobutyl-6-methyl-4-(4-methylphenyl)pyridin-3-yl]methyl}carbamate (192 mg, 0.5 mmol) and 3-methoxybenzoyl chloride (128 mg, 0.75 mmol) according to a method similar to the method of Example 223.

¹H-NMR (DMSO-d₆) δ:1.00 (6H, d, J=6.6 Hz), 2.19-2.31 (1H, m), 2.32 (3H, s), 2.58 (3H, s), 3.02 (2H, s), 3.75 (3H, s), 3.85 (2H, brs), 7.08-7.10 (2H, m), 7.18-7.36 (6H, m), 8.33 (3H, brs), 9.96 (1H, brs).

### Example 265

### N-[5-(aminomethyl)-6-isobutyl-2-methyl-4-(4-methylphenyl)pyridin-3-yl]-3-fluorobenzamide dihydrochloride

N-[5-(Aminomethyl)-6-isobutyl-2-methyl-4-(4-methylphenyl)pyridin-3-yl]-3-fluorobenzamide dihydrochloride (186 mg, yield 78%) was obtained as a white powder from tert-butyl {[5-amino-2-isobutyl-6-methyl-4-(4-methylphenyl)pyridin-3-yl]methyl}carbamate (192 mg, 0.5 mmol) and 3-fluorobenzoyl chloride (122 mg, 0.75 mmol) according to a method similar to the method of Example 223.

¹H-NMR (DMSO-d₆) δ:1.01 (6H, d, J=6.6 Hz), 2.18-2.36 (1H, m), 2.31 (3H, s), 2.62 (3H, s), 3.08 (2H, s), 3.86 (2H, s), 7.26 (4H, s), 7.38-7.42 (2H, m), 7.50 (2H, s), 8.41 (3H, brs), 10.22 (1H, brs).

### Example 266

### N-[5-(aminomethyl)-6-isobutyl-2-methyl-4-(4-methylphenyl)pyridin-3-yl]-4-methoxybenzamide dihydrochloride

N-[5-(Aminomethyl)-6-isobutyl-2-methyl-4-(4-methylphenyl)pyridin-3-yl]-4-methoxybenzamide dihydrochloride (209 mg, yield 95%) was obtained as a white powder from tert-butyl {[5-amino-2-isobutyl-6-methyl-4-(4-methylphenyl)pyridin-3-yl]methyl}carbamate (192 mg, 0.5 mmol) and 4-methoxybenzoyl chloride (128 mg, 0.75 mmol) according to a method similar to the method of Example 223.

¹H-NMR (DMSO-d₆) δ:1.00 (6H, d, J=6.6 Hz), 2.19-2.26 (1H, m), 2.31 (3H, s), 2.63 (3H, s), 3.12 (2H, s), 3.79 (3H, s), 3.87 (2H, brs), 6.96 (1H, t, J=9.0 Hz), 7.25 (4H, s), 7.67 (2H, d, J=9.0 Hz), 8.43 (3H, brs), 9.92 (1H, brs).

### Example 267

### N-[5-(aminomethyl)-6-isobutyl-2-methyl-4-(4-methylphenyl)pyridin-3-yl]-4-fluorobenzamide dihydrochloride

N-[5-(Aminomethyl)-6-isobutyl-2-methyl-4-(4-methylphenyl)pyridin-3-yl]-4-fluorobenzamide dihydrochloride (204 mg, yield 95%) was obtained as a white powder from tert-butyl {[5-amino-2-isobutyl-6-methyl-4-(4-methylphenyl)pyridin-3-yl]methyl}carbamate (192 mg, 0.5 mmol) and 4-fluorobenzoyl chloride (122 mg, 0.75 mmol) according to a method similar to the method of Example 223.

¹H-NMR (DMSO-d₆) δ:1.00 (6H, d, J=6.6 Hz), 2.14-2.31 (1H, m), 2.31 (3H, s), 2.62 (3H, s), 3.08 (2H, s), 3.85 (2H, s), 7.25-7.30 (6H, m), 7.70-7.75 (2H, m), 8.41 (3H, brs), 10.14 (1H, brs).

### Example 268

### (5-methyl-2-oxo-1,3-dioxol-4-yl)methyl 5-(aminomethyl)-6-isobutyl-2-methyl-4-(4-methylphenyl)pyridin-3-yl]acetate dihydrochloride

1) (5-Methyl-2-oxo-1,3-dioxol-4-yl)methyl [5-{[(tert-butoxycarbonyl)amino]methyl}-6-isobutyl-2-methyl-4-(4-methylphenyl)pyridin-3-yl]acetate (540 mg, yield 86%) was obtained as a white powder from [5-{[(tert-butoxycarbonyl)amino]methyl}-6-isobutyl-2-methyl-4-(4-methylphenyl)pyridin-3-yl]acetic acid (500 mg, 1.17 mmol) and 4-(chloromethyl)-5-methyl-1,3-dioxol-2-one (209 mg, 1.41 mmol) according to a method similar to the method of Example 176-1).

¹H-NMR (CDCl₃) δ:0.97 (6H, d, J=6.8 Hz), 1.38 (9H, s), 2.14 (3H, s), 2.16-2.28 (1H, m), 2.40 (3H, s), 2.49 (3H, s), 2.75 (2H, d, J=7.4 Hz), 3.40 (2H, s), 4.04 (2H, d, J=5.1 Hz), 4.21 (1H, brs), 4.76 (2H, s), 6.93 (2H, d, J=7.9 Hz), 7.21 (2H, d, J=7.9 Hz).

2) (5-Methyl-2-oxo-1,3-dioxol-4-yl)methyl 5-(aminomethyl)-6-isobutyl-2-methyl-4-(4-methylphenyl)pyridin-3-yl]acetate dihydrochloride (500 mg, yield 99%) was obtained as a white powder from (5-methyl-2-oxo-1,3-dioxol-4-yl)methyl [5-{[(tert-butoxycarbonyl)amino]methyl}-6-isobutyl-2-methyl-4-(4-methylphenyl)pyridin-3-yl]acetate (530 mg, 0.984 mmol) according to a method similar to the method of Example 2-3).

¹H-NMR (DMSO-d₆)δ:0.99 (6H, d, J=6.6 Hz), 2.15 (3H, s), 2.18-2.25 (1H, m), 2.39 (3H, s), 2.88 (3H, s), 3.29 (2H, d, J=7.2 Hz), 3.54-3.64 (4H, m), 4.94 (2H, s), 7.16 (2H, d, J=7.9 Hz), 7.33 (2H, d, J=7.9 Hz), 8.63 (3H, brs).

### Example 269

### 2-[4-(methoxycarbonyl)phenyl]ethyl 5-(aminomethyl)-6-isobutyl-2-methyl-4-(4-methylphenyl)nicotinate dihydrochloride

1) 2-[4-(Methoxycarbonyl)phenyl]ethyl 5-{[(tert-butoxycarbonyl)amino]methyl}-6-isobutyl-2-methyl-4-(4-methylphenyl)nicotinate (1.77 g, yield 70%) was obtained as a colorless oil from 5-{[(tert-butoxycarbonyl)amino]methyl}-6-isobutyl-2-methyl-4-(4-methylphenyl)nicotinic acid (1.80 g, 4.37 mmol) and methyl 4-(2-bromoethyl)benzoate (1.06 g, 4.37 mmol) according to a method similar to the method of Example 169-1).

¹H-NMR (CDCl₃) δ:0.97 (6H, d, J=6.6 Hz), 1.39 (9H, s), 2.16-2.28 (1H, m), 2.37 (3H, s), 2.46 (3H, s), 2.66 (2H, t, J=7.0 Hz), 2.77 (2H, d, J=7.4 Hz), 3.91 (3H, s), 4.11-4.15 (4H, m), 4.22 (1H, brs), 7.02 (2H, d, J=8.1 Hz), 7.15 (4H, d, J=8.3 Hz), 7.95 (2H, d, J=8.5 Hz).

2) 2-[4-(Methoxycarbonyl)phenyl]ethyl 5-(aminomethyl)-6-isobutyl-2-methyl-4-(4-methylphenyl)nicotinate dihydrochloride (291 mg, yield 82%) was obtained as a white solid from 2-[4-(methoxycarbonyl)phenyl]ethyl 5-{[(tert-butoxycarbonyl)amino]methyl}-6-isobutyl-2-methyl-4-(4-methylphenyl)nicotinate (0.37 g, 0.644 mmol) according to a method similar to the method of Example 2-3).

¹H-NMR (DMSO-d₆) δ:0.96 (6H, d, J=6.8 Hz), 2.14-2.27 (1H, m), 2.35 (3H, s), 2.42 (3H, brs), 2.73 (2H, d, J=6.4 Hz), 2.91 (2H, brs), 3.81 (2H, d, J=5.3 Hz), 3.85 (3H, s), 4.17 (2H, t, J=6.5 Hz), 7.12 (2H, d, J=6.8 Hz), 7.22 (2H, d, J=7.9 Hz), 7.29 (2H, d, J=8.3 Hz), 7.89 (2H, d, J=8.3 Hz), 8.34 (3H, brs).

### Example 270

### 4-[2-({[5-(aminomethyl)-6-isobutyl-2-methyl-4-(4-methylphenyl)pyridin-3-yl]carbonyl}oxy)ethyl]benzoic acid dihydrochloride

1) 4-[2-({[5-{[(tert-Butoxycarbonyl)amino]methyl}-6-isobutyl-2-methyl-4-(4-methylphenyl)pyridin-3-yl]carbonyl}oxy)ethyl]benzoic acid (1.30 g, yield 95%) was obtained as a colorless oil from 2-[4-(methoxycarbonyl)phenyl]ethyl 5-{[(tert-butoxycarbonyl)amino]methyl}-6-isobutyl-2-methyl-4-(4-methylphenyl)nicotinate (1.40 g, 2.44 mmol) according to a method similar to the method of Example 9-1).

¹H-NMR (CDCl₃) δ:0.97 (6H, d, J=6.8 Hz), 1.39 (9H, s), 2.16-2.27 (1H, m), 2.37 (3H, s), 2.44 (3H, s), 2.70 (2H, d, J=6.9 Hz), 2.79 (2H, d, J=7.2 Hz), 4.11-4.18 (4H, m), 4.24 (1H, brs), 7.02 (2H, d, J=7.9 Hz), 7.15-7.20 (4H, m), 8.01 (2H, d, J=8.3 Hz).

2) 4-[2-({[5-(Aminomethyl)-6-isobutyl-2-methyl-4-(4-methylphenyl)pyridin-3-yl]carbonyl}oxy)ethyl]benzoic acid dihydrochloride (359 mg, yield 94%) was obtained as a white solid from 4-[2-({[5-{[(tert-butoxycarbonyl)amino]methyl}-6-isobutyl-2-methyl-4-(4-methylphenyl)pyridin-3-yl]carbonyl}oxy)ethyl]benzoic acid (0.40 g, 0.713 mmol) according to a method similar to the method of Example 2-3).

¹H-NMR (DMSO-d₆) δ:0.96 (6H, d, J=6.6 Hz), 2.14-2.25 (1H, m), 2.35 (3H, s), 2.42 (3H, s), 2.71 (2H, t, J=6.5 Hz), 2.87 (2H, d, J=7.0 Hz), 3.80 (2H, d, J=5.3 Hz), 4.16 (2H, t, J=6.5 Hz), 7.11 (2H, d, J=8.1 Hz), 7.21-7.26 (4H, m), 7.87 (2H, d, J=8.1 Hz), 8.28 (3H, brs).

### Example 271

### 2-[4-(aminocarbonyl)phenyl]ethyl 5-(aminomethyl)-6-isobutyl-2-methyl-4-(4-methylphenyl)nicotinate dihydrochloride

1) 2-[4-(Aminocarbonyl)phenyl]ethyl 5-{[(tert-butoxycarbonyl)amino]methyl}-6-isobutyl-2-methyl-4-(4-methylphenyl)nicotinate (598 mg, yield 99%) was obtained as a colorless oil from 4-[2-({[5-{[(tert-butoxycarbonyl)amino]methyl}-6-isobutyl-2-methyl-4-(4-methylphenyl)pyridin-3-yl]carbonyl}oxy)ethyl]benzoic acid (0.60 g, 1.07 mmol) according to a method similar to the method of Example 3-1).

¹H-NMR (CDCl₃) δ:0.97 (6H, d, J=6.6 Hz), 1.39 (9H, s), 2.16-2.27 (1H, m), 2.37 (3H, s), 2.47 (3H, s), 2.66 (2H, t, J=7.1 Hz), 2.78 (2H, d, J=7.2 Hz), 4.09-4.15 (4H, m), 4.24 (1H, brs), 5.67 (1H, brs), 6.06 (1H, brs), 7.02 (2H, d, J=7.9 Hz), 7.15-7.19 (4H, m), 7.73 (2H, d, J=8.1 Hz).

2) 2-[4-(Aminocarbonyl)phenyl]ethyl 5-(aminomethyl)-6-isobutyl-2-methyl-4-(4-methylphenyl)nicotinate dihydrochloride (508 mg, yield 90%) was obtained as a white solid from 2-[4-(aminocarbonyl)phenyl]ethyl 5-{[(tert-butoxycarbonyl)amino]methyl}-6-isobutyl-2-methyl-4-(4-methylphenyl)nicotinate (598 mg, 1.06 mmol) according to a method similar to the method of Example 2-3).

¹H-NMR (DMSO-d₆) δ:0.96 (6H, d, J=6.6 Hz), 2.16-2.25 (1H, m), 2.36 (3H, s), 2.42 (3H, brs), 2.67 (2H, t, J=6.4 Hz), 2.87 (2H, brs), 3.81 (2H, d, J=5.5 Hz), 4.16 (2H, t, J=6.5 Hz), 7.11 (2H, d, J=7.7 Hz), 7.18-7.25 (4H, m), 7.32 (1H, brs), 7.81 (2H, d, J=8.3 Hz), 7.95 (1H, brs), 8.27 (3H, brs).

### Example 272

### 3-{[5-(aminomethyl)-6-isobutyl-2-methyl-4-(4-methylphenyl)pyridin-3-yl]methoxy}benzamide

1) tert-Butyl {[5-{[3-(aminocarbonyl)phenoxy]methyl}-2-isobutyl-6-methyl-4-(4-methylphenyl)pyridin-3-yl]methyl}carbamate (240 mg, yield 80%) was obtained as a white solid from 3-{[5-{[(tert-butoxycarbonyl)amino]methyl}-6-isobutyl-2-methyl-4-(4-methylphenyl)pyridin-3-yl]methoxy}benzoic acid (0.30 g, 0.578 mmol) according to a method similar to the method of Example 3-1).

¹H-NMR (CDCl₃) δ:0.99 (6H, d, J=6.6 Hz), 1.39 (9H, s), 2.21-2.28 (1H, m), 2.35 (3H, s), 2.62 (3H, s), 2.79 (2H, d, J=7.2 Hz), 4.09-4.11 (2H, m), 4.22 (1H, brs), 4.68 (2H, s), 5.55 (1H, brs), 6.01 (1H, brs), 6.96-7.01 (1H, m), 7.04 (2H, d, J=7.9 Hz), 7.17 (2H, d, J=7.7 Hz), 7.29-7.32 (2H, m), 8.02 (1H, s).

2) 3-{[5-(Aminomethyl)-6-isobutyl-2-methyl-4-(4-methylphenyl)pyridin-3-yl]methoxy}benzamide (166 mg, yield 85%) was obtained as a white solid from tert-butyl {[5-{[3-(aminocarbonyl)phenoxy]methyl}-2-isobutyl-6-methyl-4-(4-methylphenyl)pyridin-3-yl]methyl}carbamate (240 mg, 0.463 mmol) according to a method similar to the method of Example 239-2).

¹H-NMR (CDCl₃) δ:1.00 (6H, d, J=6.8 Hz), 2.21-2.30 (1H, m), 2.36 (3H, s), 2.61 (3H, s), 2.81 (2H, d, J=7.2 Hz), 3.60 (2H, s), 4.68 (2H, s), 5.52 (1H, brs), 6.06 (1H, brs), 6.96-7.00 (1H, m), 7.09 (2H, d, J=7.9 Hz), 7.18 (2H, d, J=7.9 Hz), 7.25-7.31 (3H, m).

### Example 273

### methyl 2-{[5-(aminomethyl)-6-isobutyl-2-methyl-4-(4-methylphenyl)pyridin-3-yl]methoxy}-5-methylbenzoate dihydrochloride

1) Methyl 2-{[5-{[(tert-butoxycarbonyl)amino]methyl}-6-isobutyl-2-methyl-4-(4-methylphenyl)pyridin-3-yl]methoxy}-5-methylbenzoate (720 mg, yield 52%) was obtained as a white powder from tert-butyl {[5-(hydroxymethyl)-2-isobutyl-6-methyl-4-(4-methylphenyl)pyridin-3-yl]methyl}carbamate (1.0 g, 2.51 mmol) and methyl 2-hydroxy-5-methylbenzoate (500 mg, 3.01 mmol) according to a method similar to the method of Example 214-1).

¹H-NMR (CDCl₃) δ:0.98 (6H, d, J=6.6 Hz), 1.39 (9H, s), 2.17-2.26 (1H, m), 2.27 (3H, s), 2.37 (3H, s), 2.67 (3H, s), 2.78 (2H, d, J=7.2 Hz), 3.80 (3H, s), 4.09 (2H, d, J=4.9 Hz), 4.20 (1H, brs), 4.68 (2H, s), 7.02-7.06 (3H, m), 7.11 (1H, dd, J=8.5, 1.9 Hz), 7.16 (2H, d, J=7.7 Hz), 7.52 (1H, d, J=1.9 Hz).

2) Methyl 2-{[5-(aminomethyl)-6-isobutyl-2-methyl-4-(4-methylphenyl)pyridin-3-yl]methoxy}-5-methylbenzoate dihydrochloride (100 mg, yield 70%) was obtained as a white powder from methyl 2-{[5-{[(tert-butoxycarbonyl)amino]methyl}-6-isobutyl-2-methyl-4-(4-methylphenyl)pyridin-3-yl]methoxy}-5-methylbenzoate (150 mg, 0.274 mmol) according to a method similar to the method of Example 2-3).

¹H-NMR (DMSO-d₆)δ:1.03 (6H, d, J=6.2 Hz), 2.18-2.24 (1H, m), 2.24 (3H, s), 2.37 (3H, s), 2.99 (3H, s), 3.29 (2H, d, J=7.2 Hz), 3.70-3.76 (5H, m), 4.78 (2H, s), 6.78 (1H, d, J=8.5 Hz), 7.17-7.40 (5H, m), 7.46 (1H, s), 8.63 (3H, brs).

### Example 274

### methyl 2-{[5-(aminomethyl)-6-isobutyl-2-methyl-4-(4-methylphenyl)pyridin-3-yl]methoxy}-5-chlorobenzoate dihydrochloride

1) Methyl 2-{[5-{[(tert-butoxycarbonyl)amino]methyl}-6-isobutyl-2-methyl-4-(4-methylphenyl)pyridin-3-yl]methoxy}-5-chlorobenzoate (0.80 g, yield 71%) was obtained as a white powder from tert-butyl {[5-(hydroxymethyl)-2-isobutyl-6-methyl-4-(4-methylphenyl)pyridin-3-yl]methyl}carbamate (0.80 g, 2.0 mmol) and methyl 5-chlorosalicylate (0.56 g, 3.0 mmol) according to a method similar to the method of Example 106-1).

¹H-NMR (CDCl₃)δ: 0.98 (6H, d, J=6.6 Hz), 1.39 (9H, s), 2.15-2.30 (1H, m), 2.37 (3H, s), 2.66 (3H, s), 2.78 (2H, d, J=7.2 Hz), 3.81 (3H, s), 4.09 (2H, d, J=4.9 Hz), 4.15-4.25 (1H, m), 4.69 (2H, s), 6.57 (1H, d, J=8.9 Hz), 7.03 (2H, d, J=8.0 Hz), 7.17 (2H, d, J=8.0 Hz), 7.26 (1H, dd, J=2.7, 8.9 Hz), 7.69 (1H, d, J=2.7 Hz).

2) A mixture of methyl 2-{[5-{[(tert-butoxycarbonyl)amino]methyl}-6-isobutyl-2-methyl-4-(4-methylphenyl)pyridin-3-yl]methoxy}-5-chlorobenzoate (0.19 g, 0.33 mmol) and hydrogen chloride methanol solution (4 mL) was stirred at room temperature for 3 hrs. The reaction mixture was concentrated under reduced pressure and the obtained solid was washed with diisopropyl ether to give methyl 2-{[5-(aminomethyl)-6-isobutyl-2-methyl-4-(4-methylphenyl)pyridin-3-yl]methoxy}-5-chlorobenzoate dihydrochloride (0.17 g, yield 96%) as a white powder.

¹H-NMR (DMSO-d₆)δ: 0.99 (6H, d, J=6.6 Hz), 2.15-2.30 (1H, m), 2.35 (3H, s), 3.08 (3H, brs), 3.08 (2H, brs), 3.75 (3H, s), 3.82 (2H, d, J=4.5 Hz), 4.79 (2H, s), 6.97 (1H, d, J=9.0 Hz), 7.24 (2H, d, J=7.9 Hz), 7.29 (2H, d, J=7.9 Hz), 7.52 (1H, dd, J=2.8, 9.0 Hz), 7.65 (1H, d, J=2.8 Hz), 8.35 (3H, brs).

### Example 275

### methyl 2-{[5-(aminomethyl)-6-isobutyl-2-methyl-4-(4-methylphenyl)pyridin-3-yl]methoxy}-5-methoxybenzoate dihydrochloride

1) Methyl 2-{[5-{[(tert-butoxycarbonyl)amino]methyl}-6-isobutyl-2-methyl-4-(4-methylphenyl)pyridin-3-yl]methoxy}-5-methoxybenzoate (0.70 g, yield 62%) was obtained as a white powder from tert-butyl {[5-(hydroxymethyl)-2-isobutyl-6-methyl-4-(4-methylphenyl)pyridin-3-yl]methyl}carbamate (0.80 g, 2.0 mmol) and methyl 5-methoxysalicylate (0.55 g, 3.0 mmol) according to a method similar to the method of Example 106-1).

¹H-NMR (CDCl₃)δ: 0.98 (6H, d, J=6.6 Hz), 1.39 (9H, s), 2.15-2.30 (1H, m), 2.38 (3H, s), 2.69 (3H, s), 2.78 (2H, d, J=7.2 Hz), 3.77 (3H, s), 3.81 (3H, s), 4.09 (2H, d, J=4.7 Hz), 4.15-4.30 (1H, m), 4.68 (2H, s), 6.50 (1H, d, J=9.0 Hz), 6.85 (1H, dd, J=3.2, 9.0 Hz), 7.01 (2H, d, J=7.9 Hz), 7.17 (2H, d, J=7.9 Hz), 7.24 (1H, d, J=3.2 Hz).

2) Methyl 2-{[5-(aminomethyl)-6-isobutyl-2-methyl-4-(4-methylphenyl)pyridin-3-yl]methoxy}-5-methoxybenzoate dihydrochloride (0.20 g, yield 96%) was obtained as a white powder from methyl 2-{[5-{[(tert-butoxycarbonyl)amino]methyl}-6-isobutyl-2-methyl-4-(4-methylphenyl)pyridin-3-yl]methoxy}-5-methoxybenzoate (0.23 g, 0.40 mmol) according to a method similar to the method of Example 274-2).

¹H-NMR (DMSO-d₆)δ: 0.98 (6H, d, J=6.6 Hz), 2.15-2.30 (1H, m), 2.37 (3H, s), 2.73 (3H, brs), 2.93 (2H, brs), 3.72 (3H, s), 3.73 (3H, s), 3.79 (2H, d, J=4.9 Hz), 4.69 (2H, brs), 6.77 (1H, d, J=9.0 Hz), 7.01 (1H, dd, J=3.2, 9.0 Hz), 7.14 (1H, d, J=3.2 Hz), 7.20 (2H, d, J=7.8 Hz), 7.29 (2H, d, J=7.8 Hz), 8.11 (3H, brs).

### Example 276

### 2-{[5-(aminomethyl)-6-isobutyl-2-methyl-4-(4-methylphenyl)pyridin-3-yl]methoxy}-4-methoxybenzoic acid dihydrochloride

1) Methyl 2-{[5-{[(tert-butoxycarbonyl)amino]methyl}-6-isobutyl-2-methyl-4-(4-methylphenyl)pyridin-3-yl]methoxy}-4-methoxybenzoate (0.81 g, yield 72%) was obtained as a white powder from tert-butyl {[5-(hydroxymethyl)-2-isobutyl-6-methyl-4-(4-methylphenyl)pyridin-3-yl]methyl}carbamate (0.80 g, 2.0 mmol) and methyl 4-methoxysalicylate (0.55 g, 3.0 mmol) according to a method similar to the method of Example 106-1).

¹H-NMR (CDCl₃)δ: 0.98 (6H, d, J=6.6 Hz), 1.39 (9H, s), 2.15-2.30 (1H, m), 2.36 (3H, s), 2.68 (3H, s), 2.78 (2H, d, J=7.2 Hz), 3.75 (3H, s), 3.77 (3H, s), 4.09 (2H, d, J=4.7 Hz), 4.20-4.25 (1H, m), 4.68 (2H, s), 6.14 (1H, d, J=2.4 Hz), 6.48 (1H, dd, J=2.4, 8.7 Hz), 7.00-7.10 (2H, m), 7.15-7.20 (2H, m, 7.79 (1H, d, J=8.7 Hz).

2) 2-{[5-{[(tert-Butoxycarbonyl)amino]methyl}-6-isobutyl-2-methyl-4-(4-methylphenyl)pyridin-3-yl]methoxy}-4-methoxybenzoic acid (0.19 g, yield 37%) was obtained as a white powder from methyl 2-{[5-{[(tert-butoxycarbonyl)amino]methyl}-6-isobutyl-2-methyl-4-(4-methylphenyl)pyridin-3-yl]methoxy}-4-methoxybenzoate (0.51 g, 0.91 mmol) according to a method similar to the method of Example 36-1).

¹H-NMR (CDCl₃)δ: 0.99 (6H, d, J=6.8 Hz), 1.39 (9H, s), 2.15-2.35 (1H, m), 2.35 (3H, s), 2.64 (3H, s), 2.81 (2H, d, J=7.2 Hz), 3.82 (3H, s), 4.09 (2H, d, J=4.9 Hz), 4.15-4.30 (1H, m), 4.87 (2H, s), 6.30 (1H, d, J=2.3 Hz), 6.63 (1H, dd, J=2.3, 8.9 Hz), 7.00 (2H, d, J=7.9 Hz), 7.18 (2H, d, J=7.9 Hz), 8.12 (1H, d, J=8.9 Hz), 10.42 (1H, brs).

3) A mixture of 2-{[5-{[(tert-butoxycarbonyl)amino]methyl}-6-isobutyl-2-methyl-4-(4-methylphenyl)pyridin-3-yl]methoxy}-4-methoxybenzoic acid (0.15 g, 0.28 mmol) and 6N hydrochloric acid (4 mL) was stirred at room temperature for 6 hrs. The reaction mixture was concentrated under reduced pressure and the obtained solid was washed with acetonitrile to give 2-{[5-(aminomethyl)-6-isobutyl-2-methyl-4-(4-methylphenyl)pyridin-3-yl]methoxy}-4-methoxybenzoic acid dihydrochloride (0.12 g, yield 81%) as a white powder.

¹H-NMR (DMSO-d₆)δ: 0.99 (6H, d, J=6.6 Hz), 2.10-2.30 (1H, m), 2.37 (3H, s), 2.86 (3H, brs), 3.06 (2H, brs), 3.73 (3H, s), 3.82 (2H, brs), 4.76 (2H, brs), 6.31 (1H, d, J=2.1 Hz), 6.60 (1H, dd, J=2.1, 8.7 Hz), 7.26 (2H, d, J=7.2 Hz), 7.32 (2H, d, J=7.2 Hz), 7.68 (1H, d, J=8.7 Hz), 8.28 (3H, brs).

### Example 277

### methyl 6-({[5-(aminomethyl)-6-isobutyl-2-methyl-4-(4-methylphenyl)pyridin-3-yl]methoxy}methyl)nicotinate trihydrochloride

1) A mixture of tert-butyl {[5-(hydroxymethyl)-2-isobutyl-6-methyl-4-(4-methylphenyl)pyridin-3-yl]methyl}carbamate (1.50 g, 3.76 mmol), triethylamine (1.05 mL, 7.52 mmol) and tetrahydrofuran (50 mL) was cooled to 0° C. and methanesulfonyl chloride (647 mg, 5.65 mmol) was added dropwise. After stirring at room temperature for 30 min., the reaction mixture was poured into saturated aqueous sodium hydrogen carbonate, and the mixture was extracted with ethyl acetate. The extract was dried over anhydrous magnesium sulfate and the solvent was evaporated under reduced pressure to give [5-{[(tert-butoxycarbonyl)amino]methyl}-6-isobutyl-2-methyl-4-(4-methylphenyl)pyridin-3-yl]methyl methanesulfonate as a crude product. The crude product was added to a solution of (5-bromopyridin-2-yl)methanol (848 mg, 4.51 mmol) and sodium hydride (60% in oil, 226 mg, 5.65 mmol) in tetrahydrofuran (50 mL) and the mixture was stirred at 60° C. for 1 hr. The reaction mixture was diluted with ethyl acetate, washed with saturated brine and dried over anhydrous magnesium sulfate. The solvent was evaporated under reduced pressure and the obtained residue was purified by silica gel column chromatography to give tert-butyl {[5-{[(5-bromopyridin-2-yl)methoxy]methyl}-2-isobutyl-6-methyl-4-(4-methylphenyl)pyridin-3-yl]methyl}carbamate (1.35 g, yield 63%) as a white solid.

¹H-NMR (CDCl₃) δ:0.97 (6H, d, J=6.6 Hz), 1.38 (9H, s), 2.15-2.24 (1H, m), 2.41 (3H, s), 2.65 (3H, s), 2.75 (2H, d, J=7.4 Hz), 4.06 (2H, d, J=4.9 Hz), 4.23 (2H, s), 4.39 (2H, s), 7.01 (2H, d, J=7.9 Hz), 7.16-7.20 (3H, m), 7.73 (1H, dd, J=8.4, 2.4 Hz), 8.54 (1H, d, J=2.1 Hz).

2) Methyl 6-({[5-{[(tert-butoxycarbonyl)amino]methyl}-6-isobutyl-2-methyl-4-(4-methylphenyl)pyridin-3-yl]methoxy}methyl)nicotinate (1.15 g, yield 88%) was obtained as a yellow oil from tert-butyl {[5-{[(5-bromopyridin-2-yl)methoxy]methyl}-2-isobutyl-6-methyl-4-(4-methylphenyl)pyridin-3-yl]methyl}carbamate (1.35 g, 2.37 mmol) according to a method similar to the method of Example 231-2).

¹H-NMR (CDCl₃) δ:0.97 (6H, d, J=6.6 Hz), 1.38 (9H, s), 2.16-2.25 (1H, m), 2.40 (3H, s, 2.67 (3H, s), 2.76 (2H, d, J=7.2 Hz), 3.95 (3H, s), 4.06 (2H, d, J=4.9 Hz), 4.20 (1H, brs), 4.27 (2H, s), 4.50 (2H, s), 7.02 (2H, d, J=7.9 Hz), 7.19 (2H, d, J=7.7 Hz), 7.36 (1H, d, J=8.1 Hz), 8.21 (1H, dd, J=8.1, 2.1 Hz), 9.08 (1H, d, J=1.7 Hz).

3) Methyl 6-({[5-(aminomethyl)-6-isobutyl-2-methyl-4-(4-methylphenyl)pyridin-3-yl]methoxy}methyl)nicotinate trihydrochloride (114 mg, yield 58%) was obtained as a white solid from methyl 6-({[5-{[(tert-butoxycarbonyl)amino]methyl}-6-isobutyl-2-methyl-4-(4-methylphenyl)pyridin-3-yl]methoxy}methyl)nicotinate (0.19 g, 0.347 mmol) according to a method similar to the method of Example 2-3).

¹H-NMR (DMSO-d₆) δ:0.98 (6H, d, J=6.6 Hz), 2.11-2.22 (1H, m), 2.38 (3H, s), 3.14 (2H, brs), 3.81 (2H, d, J=5.3 Hz), 3.90 (3H, s), 4.29 (2H, s), 4.51 (2H, s), 7.23 (2H, d, J=7.9 Hz), 7.32 (2H, d, J=7.9 Hz), 7.38 (1H, d, J=8.1 Hz), 8.25 (1H, dd, J=8.1, 2.2 Hz), 8.38 (3H, brs), 8.98 (1H, d, J=1.5 Hz).

### Example 278

### 6-({[5-(aminomethyl)-6-isobutyl-2-methyl-4-(4-methylphenyl)pyridin-3-yl]methoxy}methyl)nicotinic acid trihydrochloride

1) 6-({[5-{[(tert-Butoxycarbonyl)amino]methyl}-6-isobutyl-2-methyl-4-(4-methylphenyl)pyridin-3-yl]methoxy}methyl)nicotinic acid (760 mg, yield 81%) was obtained as a colorless oil from methyl 6-({[5-{[(tert-butoxycarbonyl)amino]methyl}-6-isobutyl-2-methyl-4-(4-methylphenyl)pyridin-3-yl]methoxy}methyl)nicotinate (0.96 g, 1.75 mmol) according to a method similar to the method of Example 9-1).

¹H-NMR (CDCl₃) δ:0.97 (6H, d, J=6.6 Hz), 1.38 (9H, s), 2.14-2.26 (1H, m), 2.39 (3H, s), 2.71 (3H, s), 2.85 (2H, d, J=7.2 Hz), 4.05-4.10 (2H, m), 4.29 (3H, brs), 4.52 (2H, s), 7.03 (2H, d, J=7.9 Hz), 7.38 (1H, d, J=8.1 Hz), 8.29 (1H, dd, J=8.2, 1.8 Hz), 9.15 (1H, d, J=1.5 Hz).

2) 6-({[5-(Aminomethyl)-6-isobutyl-2-methyl-4-(4-methylphenyl)pyridin-3-yl]methoxy}methyl)nicotinic acid trihydrochloride (259 mg, yield 90%) was obtained as a white solid from 6-({[5-{[(tert-butoxycarbonyl)amino]methyl}-6-isobutyl-2-methyl-4-(4-methylphenyl)pyridin-3-yl]methoxy}methyl)nicotinic acid (0.28 g, 0.525 mmol) according to a method similar to the method of Example 2-3).

¹H-NMR (DMSO-d₆) δ:0.98 (6H, d, J=6.4 Hz), 2.11-2.22 (1H, m), 2.39 (3H, s), 2.94 (3H, brs), 3.13-3.22 (2H, m), 3.81 (2H, brs), 4.29 (2H, brs), 4.51 (2H, s), 7.19-7.25 (2H, m), 7.30-7.36 (3H, m), 8.19-8.24 (1H, m), 8.43 (3H, brs), 8.93-8.96 (1H, m).

### Example 279

### methyl 2-{2-[5-(aminomethyl)-6-isobutyl-2-methyl-4-(4-methylphenyl)pyridin-3-yl]ethyl}benzoate dihydrochloride

1) To a solution of tert-butyl {[5-formyl-2-isobutyl-6-methyl-4-(4-methylphenyl)pyridin-3-yl]methyl}carbamate (0.36 g, 0.908 mmol) and diethyl (2-bromobenzyl)phosphonate (363 mg, 1.18 mmol) in N,N-dimethylformamide (10 mL) was added sodium methoxide (165 mg, 4.08 mmol) and the mixture was stirred at room temperature for 1 hr. The reaction mixture was diluted with ethyl acetate, washed with saturated brine and dried over anhydrous magnesium sulfate. The solvent was evaporated under reduced pressure and the obtained residue was purified by silica gel column chromatography to give tert-butyl {[5-[(E)-2-(2-bromophenyl)vinyl]-2-isobutyl-6-methyl-4-(4-methylphenyl)pyridin-3-yl]methyl}carbamate (390 mg, yield 78%) as a white solid.

¹H-NMR (CDCl₃) δ:1.00 (6H, d, J=6.6 Hz), 1.39 (9H, s), 2.18-2.30 (1H, m), 2.39 (3H, s), 2.72 (3H, s), 2.78 (2H, d, J=7.4 Hz), 4.11 (2H, d, J=5.1 Hz), 4.24 (1H, brs), 6.55 (1H, d, J=16.6 Hz), 6.78 (1H, d, J=16.6 Hz), 7.02 (2H, d, J=7.9 Hz), 7.05-7.08 (1H, m), 7.15-7.18 (2H, m), 7.22 (2H, d, J=7.7 Hz), 7.50 (1H, d, J=7.5 Hz).

2) Methyl 2-{(E)-2-[5-{[(tert-butoxycarbonyl)amino]methyl}-6-isobutyl-2-methyl-4-(4-methylphenyl)pyridin-3-yl]vinyl}benzoate (280 mg, yield 74%) was obtained as a yellow oil from tert-butyl {[5-[(E)-2-(2-bromophenyl)vinyl]-2-isobutyl-6-methyl-4-(4-methylphenyl)pyridin-3-yl]methyl}carbamate (390 mg, 0.907 mmol) according to a method similar to the method of Example 231-2).

¹H-NMR (CDCl₃) δ:0.99 (6H, d, J=6.6 Hz), 1.39 (9H, s), 2.18-2.27 (1H, m), 2.39 (3H, s), 2.74 (3H, s), 2.78 (2H, d, J=7.4 Hz), 3.89 (3H, s), 4.11 (2H, d, J=5.3 Hz), 4.24 (1H, brs), 6.47 (1H, d, J=16.8 Hz), 7.02 (2H, d, J=7.9 Hz), 7.13 (1H, d, J=7.5 Hz), 7.20-7.29 (4H, m), 7.35-7.40 (1H, m), 7.86 (1H, dd, J=7.8, 1.4 Hz).

3) A mixture of methyl 2-{(E)-2-[5-{[(tert-butoxycarbonyl)amino]methyl}-6-isobutyl-2-methyl-4-(4-methylphenyl)pyridin-3-yl]vinyl}benzoate (0.28 g, 0.53 mmol), 10% palladium-carbon (57 mg, 0.053 mmol) and methanol (10 mL) was stirred in a sealed tube under a 0.5 Mpa hydrogen atmosphere at room temperature for 3 hrs. The reaction mixture was filtered and the filtrate was concentrated under reduced pressure. The obtained residue was purified by silica gel column chromatography to give methyl 2-{2-[5-{[(tert-butoxycarbonyl)amino]methyl}-6-isobutyl-2-methyl-4-(4-methylphenyl)pyridin-3-yl]ethyl}benzoate (250 mg, yield 88%) as a white solid.

¹H-NMR (CDCl₃) δ:0.97 (6H, d, J=6.6 Hz), 1.39 (9H, s), 2.14-2.23 (1H, m), 2.43 (3H, s), 2.60 (3H, s), 2.62-2.68 (2H, m), 2.73 (2H, d, J=7.4 Hz), 2.91-2.96 (2H, m), 3.82 (3H, s), 4.01 (2H, d, J=5.1 Hz), 4.21 (1H, brs), 6.54 (1H, dd, J=7.4, 1.2 Hz), 6.94 (2H, d, J=8.1 Hz), 7.15-7.25 (4H, m), 7.77 (1H, dd, J=7.6, 1.6 Hz).

4) Methyl 2-{2-[5-(aminomethyl)-6-isobutyl-2-methyl-4-(4-methylphenyl)pyridin-3-yl]ethyl}benzoate dihydrochloride (201 mg, yield 84%) was obtained as a white solid from methyl 2-{2-[5-{[(tert-butoxycarbonyl)amino]methyl}-6-isobutyl-2-methyl-4-(4-methylphenyl)pyridin-3-yl]ethyl}benzoate (0.25 g, 0.471 mmol) according to a method similar to the method of Example 2-3).

¹H-NMR (DMSO-d₆) δ:0.99 (6H, d, J=6.6 Hz), 2.11-2.20 (1H, m), 2.45 (3H, s), 2.63-2.72 (2H, m), 2.83-2.90 (5H, m), 2.91-2.96 (2H, m), 3.18 (2H, brs), 3.73-3.84 (5H, m), 6.65 (1H, d, J=7.4 Hz), 7.26 (2H, d, J=7.7 Hz), 7.31 (1H, dd, J=7.4, 1.4 Hz), 7.35 (1H, dd, J=7.4, 1.8 Hz), 7.42 (2H, d, J=7.9 Hz), 7.75 (1H, dd, J=7.5, 1.5 Hz), 8.46 (3H, brs).

### Example 280

### methyl 4-[({[5-(aminomethyl)-6-isobutyl-2-methyl-4-(4-methylphenyl)pyridin-3-yl]acetyl}oxy)methyl]benzoate dihydrochloride

1) Methyl 4-[({[5-{[(tert-butoxycarbonyl)amino]methyl}-6-isobutyl-2-methyl-4-(4-methylphenyl)pyridin-3-yl]acetyl}oxy)methyl]benzoate (258 mg, yield 64%) was obtained as a white powder from [5-{[(tert-butoxycarbonyl)amino]methyl}-6-isobutyl-2-methyl-4-(4-methylphenyl)pyridin-3-yl]acetic acid (300 mg, 0.703 mmol) and methyl 4-(bromomethyl)benzoate (209 mg, 0.914 mmol) according to a method similar to the method of Example 169-1).

¹H-NMR (CDCl₃) δ:0.97 (6H, d, J=6.8 Hz), 1.39 (9H, s), 2.17-2.26 (1H, m), 2.38 (3H, s), 2.49 (3H, s), 2.77 (2H, d, J=7.0 Hz), 3.42 (3H, s), 3.93 (3H, s), 4.03 (2H, d, J=5.1 Hz), 5.09 (2H, s), 6.92 (2H, d, J=8.1 Hz), 7.16 (2H, d, J=8.1 Hz), 7.28 (2H, d, J=8.1 Hz), 8.01 (2H, d, J=8.1 Hz).

2) Methyl 4-[({[5-(aminomethyl)-6-isobutyl-2-methyl-4-(4-methylphenyl)pyridin-3-yl]acetyl}oxy)methyl]benzoate dihydrochloride (60 mg, yield 92%) was obtained as a white powder from methyl 4-[({[5-{[(tert-butoxycarbonyl)amino]methyl}-6-isobutyl-2-methyl-4-(4-methylphenyl)pyridin-3-yl]acetyl}oxy)methyl]benzoate (68.6 mg, 0.119 mmol) according to a method similar to the method of Example 2-3).

¹H-NMR (DMSO-d₆)δ:0.98 (6H, d, J=6.6 Hz), 2.17-2.23 (1H, m), 2.38 (3H, s), 2.85 (3H, s), 3.25 (2H, d, J=6.8 Hz), 3.63 (2H, s), 3.79 (2H, d, J=4.5 Hz), 3.87 (3H, s), 5.13 (2H, s), 7.13 (2H, d, J=7.9 Hz), 7.30 (2H, d, J=7.9 Hz), 7.39 (2H, d, J=8.3 Hz), 7.97 (2H, d, J=8.3 Hz), 8.63 (3H, brs).

### Example 281

### 2-{[5-(aminomethyl)-6-isobutyl-2-methyl-4-(4-methylphenyl)pyridin-3-yl]methoxy}-5-methylbenzoic acid dihydrochloride

1) 2-{[5-{[(tert-Butoxycarbonyl)amino]methyl}-6-isobutyl-2-methyl-4-(4-methylphenyl)pyridin-3-yl]methoxy}-5-methylbenzoic acid (450 mg, yield 86%) was obtained as a white powder from methyl 2-{[5-{[(tert-butoxycarbonyl)amino]methyl}-6-isobutyl-2-methyl-4-(4-methylphenyl)pyridin-3-yl]methoxy}-5-methylbenzoate (537 mg, 0.982 mmol) according to a method similar to the method of Example 9-1).

¹H-NMR (CDCl₃) δ:0.99 (6H, d, J=6.6 Hz), 1.39 (9H, s), 2.18-2.30 (1H, m), 2.32 (3H, s), 2.34 (3H, s), 2.64 (3H, s), 2.80 (2H, d, J=7.4 Hz), 4.10 (2H, d, J=4.9 Hz), 4.20 (1H, s), 4.88 (2H, s), 6.72 (1H, d, J=8.5 Hz), 7.01 (2H, d, J=8.1 Hz), 7.18 (2H, d, J=8.1 Hz), 7.23-7.25 (1H, m), 7.97 (1H, d, J=2.26 Hz).

2) 2-{[5-(Aminomethyl)-6-isobutyl-2-methyl-4-(4-methylphenyl)pyridin-3-yl]methoxy}-5-methylbenzoic acid dihydrochloride (150 mg, yield 94%) was obtained as a white powder from 2-{[5-{[(tert-butoxycarbonyl)amino]methyl}-6-isobutyl-2-methyl-4-(4-methylphenyl)pyridin-3-yl]methoxy}-5-methylbenzoic acid (168 mg, 0.316 mmol) according to a method similar to the method of Example 2-3).

¹H-NMR (DMSO-d₆)δ:1.02 (6H, d, J=6.6 Hz), 2.18-2.30 (1H, m), 2.24 (3H, s), 2.38 (3H, s), 3.00 (3H, s), 3.30 (2H, d, J=6.8 Hz), 3.87 (2H, d, J=2.6 Hz), 4.78 (2H, s), 6.72 (1H, d, J=8.5 Hz), 7.20-7.22 (1H, m), 7.30-7.34 (4H, m), 7.43 (1H, d, J=1.5 Hz), 8.63 (3H, brs).

### Example 282

### methyl 3-[({[5-(aminomethyl)-6-isobutyl-2-methyl-4-(4-methylphenyl)pyridin-3-yl]acetyl}oxy)methyl]benzoate dihydrochloride

1) Methyl 3-[({[5-{[(tert-butoxycarbonyl)amino]methyl}-6-isobutyl-2-methyl-4-(4-methylphenyl)pyridin-3-yl]acetyl}oxy)methyl]benzoate (401 mg, yield 64%) was obtained as a white powder from [5-{[(tert-butoxycarbonyl)amino]methyl}-6-isobutyl-2-methyl-4-(4-methylphenyl)pyridin-3-yl]acetic acid (466 mg, 1.09 mmol) and methyl 3-(bromomethyl)benzoate (325 mg, 1.42 mmol) according to a method similar to the method of Example 169-1).

¹H-NMR (CDCl₃) δ:0.96 (6H, d, J=6.6 Hz), 1.38 (9H, s), 2.17-2.26 (1H, m), 2.36 (3H, s), 2.48 (3H, s), 2.74 (2H, d, J=7.4 Hz), 3.41 (2H, s), 3.93 (3H, s), 4.03 (2H, d, J=4.9 Hz), 4.20 (1H, brs), 5.08 (2H, s), 6.90-6.93 (2H, m), 7.14 (2H, d, J=7.7 Hz), 7.40-7.44 (2H, m), 7.93 (1H, d, J=0.8 Hz), 7.98-8.01 (1H, m).

2) Methyl 3-[({[5-(aminomethyl)-6-isobutyl-2-methyl-4-(4-methylphenyl)pyridin-3-yl]acetyl}oxy)methyl]benzoate dihydrochloride (80 mg, yield 99%) was obtained as a white powder from methyl 3-[({[5-{[(tert-butoxycarbonyl)amino]methyl}-6-isobutyl-2-methyl-4-(4-methylphenyl)pyridin-3-yl]acetyl}oxy)methyl]benzoate (84.6 mg, 0.147 mmol) according to a method similar to the method of Example 2-3).

¹H-NMR (DMSO-d₆)δ:0.98 (6H, d, J=6.6 Hz), 2.17-2.26 (1H, m), 2.36 (3H, s), 2.88 (3H, s), 3.30 (2H, d, J=6.8 Hz), 3.60 (2H, s), 3.80 (2H, d, J=3.8 Hz), 3.88 (3H, s), 5.13 (2H, s), 7.12 (2H, d, J=7.9 Hz), 7.27 (2H, d, J=7.9 Hz), 7.56-7.60 (2H, m), 7.89 (1H, s), 7.95-7.98 (1H, m), 8.63 (3H, brs).

### Example 283

### 2-{[5-(aminomethyl)-6-isobutyl-2-methyl-4-(4-methylphenyl)pyridin-3-yl]methoxy}-4-methoxybenzamide dihydrochloride

1) tert-Butyl {[5-{[2-(aminocarbonyl)-5-methoxyphenoxy]methyl}-2-isobutyl-6-methyl-4-(4-methylphenyl)pyridin-3-yl]methyl}carbamate (0.31 g, yield 82%) was obtained as a white powder from 2-{[5-{[(tert-butoxycarbonyl)amino]methyl}-6-isobutyl-2-methyl-4-(4-methylphenyl)pyridin-3-yl]methoxy}-4-methoxybenzoic acid (0.38 g, 0.68 mmol) according to a method similar to the method of Example 3-1).

¹H-NMR (CDCl₃)δ: 0.99 (6H, d, J=6.6 Hz), 1.39 (9H, s), 2.15-2.30 (1H, m), 2.36 (3H, s), 2.63 (3H, s), 2.80 (2H, d, J=7.2 Hz), 3.80 (3H, s), 4.10 (2H, d, J=5.1 Hz), 4.20-4.25 (1H, m), 4.75 (2H, s), 5.51 81H, brs), 6.26 (1H, d, J=2.3 Hz), 6.58 (1H, dd, J=2.3, 8.9 Hz), 7.00 (2H, d, J=7.9 Hz), 7.18 (2H, d, J=7.9 Hz), 7.41 (1H, brs), 8.18 (1H, d, J=8.9 Hz).

2) 2-{[5-(Aminomethyl)-6-isobutyl-2-methyl-4-(4-methylphenyl)pyridin-3-yl]methoxy}-4-methoxybenzamide dihydrochloride (0.22 g, yield 91%) was obtained as a white powder from tert-butyl {[5-{[2-(aminocarbonyl)-5-methoxyphenoxy]methyl}-2-isobutyl-6-methyl-4-(4-methylphenyl)pyridin-3-yl]methyl}carbamate (0.25 g, 0.46 mmol) according to a method similar to the method of Example 2-3).

¹H-NMR (DMSO-d₆)δ: 0.99 (6H, d, J=6.6 Hz), 2.10-2.30 (1H, m), 2.35 (3H, s), 2.78 (3H, brs), 3.01 (2H, brs), 3.74 (3H, s), 3.80 (2H, d, J=5.1 Hz), 4.82 (2H, s), 6.42 (1H, d, J=2.2 Hz), 6.63 (1H, dd, J=2.2, 8.7 Hz), 7.14 (2H, brs), 7.15-7.35 (4H, m), 7.74 (1H, d, J=8.7 Hz), 8.28 (3H, brs).

### Example 284

### methyl 3-{[5-(aminomethyl)-6-isobutyl-2-methyl-4-(4-methylphenyl)pyridin-3-yl]methoxy}-2-naphthoate dihydrochloride

1) Methyl 3-{[5-{[(tert-butoxycarbonyl)amino]methyl}-6-isobutyl-2-methyl-4-(4-methylphenyl)pyridin-3-yl]methoxy}-2-naphthoate (1.07 g, yield 73%) was obtained as a white powder from tert-butyl {[5-(hydroxymethyl)-2-isobutyl-6-methyl-4-(4-methylphenyl)pyridin-3-yl]methyl}carbamate (1.0 g, 2.51 mmol) and methyl 3-hydroxy-2-naphthoate (609 mg, 3.01 mmol) according to a method similar to the method of Example 214-1).

¹H-NMR (CDCl₃) δ:1.00 (6H, d, J=6.6 Hz), 1.38 (9H, s), 2.18-2.31 (1H, m), 2.34 (3H, s), 2.70 (3H, s), 2.79 (2H, d, J=7.4 Hz), 3.87 (3H, s), 4.11 (2H, d, J=4.7 Hz), 4.20 (1H, brs), 4.81 (2H, s), 6.91 (1H, s), 7.09 (2H, d, J=7.9 Hz), 7.16 (2H, d, J=7.9 Hz), 7.34-7.38 (1H, m), 7.46-7.50 (1H, m), 7.58-7.62 (1H, m), 7.79 (1H, d, J=8.1 Hz), 8.22 (1H, s).

2) Methyl 3-{[5-(aminomethyl)-6-isobutyl-2-methyl-4-(4-methylphenyl)pyridin-3-yl]methoxy}-2-naphthoate dihydrochloride (178 mg, yield 84%) was obtained as a white powder from methyl 3-{[5-{[(tert-butoxycarbonyl)amino]methyl}-6-isobutyl-2-methyl-4-(4-methylphenyl)pyridin-3-yl]methoxy}-2-naphthoate (220 mg, 0.378 mmol) according to a method similar to the method of Example 2-3).

¹H-NMR (DMSO-d₆)δ:1.05 (6H, d, J=6.2 Hz), 2.18-2.33 (1H, m), 2.34 (3H, s), 3.06 (3H, s), 3.36 (2H, d, J=6.0 Hz), 3.84 (3H, s), 3.91 (2H,s), 4.96 (2H, s), 7.35-7.45 (6H, m), 7.58 (1H, t, J=7.35 Hz), 7.79 (1H, d, J=8.1 Hz), 7.98 (1H, d, J=7.9 Hz), 8.32 (1H, s), 8.63 (3H, brs).

### Example 285

### 3-{[5-(aminomethyl)-6-isobutyl-2-methyl-4-(4-methylphenyl)pyridin-3-yl]methoxy}-2-naphthoic acid dihydrochloride

1) 3-{[5-{[(tert-Butoxycarbonyl)amino]methyl}-6-isobutyl-2-methyl-4-(4-methylphenyl)pyridin-3-yl]methoxy}-2-naphthoic acid (860 mg, yield 100%) was obtained as a white powder from methyl 3-{[5-{[(tert-butoxycarbonyl)amino]methyl}-6-isobutyl-2-methyl-4-(4-methylphenyl)pyridin-3-yl]methoxy}-2-naphthoate (817 mg, 1.40 mmol) according to a method similar to the method of Example 9-1).

¹H-NMR (CDCl₃) δ:1.02 (6H, d, J=6.6 Hz), 1.38 (9H, s), 2.20-2.30 (1H, m), 2.32 (3H, s), 2.81 (3H, s), 2.97 (2H, d, J=6.4 Hz), 4.15 (2H, d, J=3.0 Hz), 4.20 (1H, brs), 5.01 (2H, s), 7.06 (3H, d, J=7.7 Hz), 7.18 (2H, d, J=7.7 Hz) , 7.40-7.48 (1H, m), 7.52-7.58 (1H, m), 7.62-7.68 (1H, m), 7.89 (1H, d, J=8.1 Hz), 8.67 (1H, s).

2) 3-{[5-(Aminomethyl)-6-isobutyl-2-methyl-4-(4-methylphenyl)pyridin-3-yl]methoxy}-2-naphthoic acid dihydrochloride (300 mg, yield 98%) was obtained as a white powder from 3-{[5-{[(tert-butoxycarbonyl)amino]methyl}-6-isobutyl-2-methyl-4-(4-methylphenyl)pyridin-3-yl]methoxy}-2-naphthoic acid (320 mg, 0.563 mmol) according to a method similar to the method of Example 2-3).

¹H-NMR (DMSO-d₆)δ:1.00 (6H, d, J=6.4 Hz), 2.17-2.29 (1H, m), 2.33 (3H, s), 2.81 (3H, s), 2.90 (2H, s), 3.83 (2H, s), 4.86 (2H, s), 7.24 (1H, s), 7.26-7.33 (4H, m), 7.41 (1H, t, J=7.5 Hz), 7.53 (1H, t, J=7.5 Hz), 7.75 (1H, d, J=8.1 Hz), 7.94 (1H, d, J=8.1 Hz), 8.52 (1H, s), 8.63 (3H, brs).

### Example 286

### 2-{[5-(aminomethyl)-6-isobutyl-2-methyl-4-(4-methylphenyl)pyridin-3-yl]methoxy}-5-methylbenzamide dihydrochloride

1) tert-Butyl {[5-{[2-(aminocarbonyl)-4-methylphenoxy]methyl}-2-isobutyl-6-methyl-4-(4-methylphenyl)pyridin-3-yl]methyl}carbamate (250 mg, yield 91%) was obtained as a white powder from 2-{[5-{[(tert-butoxycarbonyl)amino]methyl}-6-isobutyl-2-methyl-4-(4-methylphenyl)pyridin-3-yl]methoxy}-5-methylbenzoic acid (276 mg, 0.518 mmol) according to a method similar to the method of Example 3-1).

¹H-NMR (CDCl₃) δ:0.99 (6H, d, J=6.6 Hz), 1.39 (9H, s), 2.17-2.28 (1H, m), 2.31 (3H, s), 2.35 (3H, s), 2.64 (3H, s), 2.81 (2H, s), 4.11 (2H, s), 4.20 (1H, s), 4.76 (2H, s), 6.66 (1H, d, J=8.5 Hz), 7.00 (2H, d, J=8.1 Hz), 7.17 (2H, d, J=8.1 Hz), 7.55 (2H, s), 8.00 (2H, s).

2) 2-{[5-(Aminomethyl)-6-isobutyl-2-methyl-4-(4-methylphenyl)pyridin-3-yl]methoxy}-5-methylbenzamide dihydrochloride (200 mg, yield 92%) was obtained as a white powder from tert-butyl {[5-{[2-(aminocarbonyl)-4-methylphenoxy]methyl}-2-isobutyl-6-methyl-4-(4-methylphenyl)pyridin-3-yl]methyl}carbamate (230 mg, 0.433 mmol) according to a method similar to the method of Example 2-3).

¹H-NMR (DMSO-d₆)δ:1.01 (6H, d, J=6.4 Hz), 2.10-2.30 (4H, m), 2.36 (3H, s), 2.96 (3H, s), 3.27 (2H, d, J=7.0 Hz), 3.86 (2H, d, J=4.5 Hz), 4.72-4.84 (2H, m), 6.76 (1H, d, J=8.5 Hz), 7.15 (1H, dd, J=8.5, 1.9 Hz), 7.25-7.38 (4H, m), 7.42 (1H, d, J=1.9 Hz), 8.64 (3H, brs).

### Example 287

### N-[5-(aminomethyl)-6-isobutyl-2-methyl-4-(4-methylphenyl)pyridin-3-yl]acetamide dihydrochloride

N-[5-(Aminomethyl)-6-isobutyl-2-methyl-4-(4-methylphenyl)pyridin-3-yl]acetamide dihydrochloride (198 mg, yield 95%) was obtained as a white powder from tert-butyl {[5-amino-2-isobutyl-6-methyl-4-(4-methylphenyl)pyridin-3-yl]methyl}carbamate (192 mg, 0.5 mmol) and acetyl chloride (53 μL, 0.75 mmol) according to a method similar to the method of Example 223.

¹H-NMR (DMSO-d₆) δ:0.98 (6H, d, J=6.6 Hz), 1.76 (3H, s), 2.13-2.22 (1H, m), 2.39 (3H, s), 2.55 (3H, s), 3.02 (2H, brs), 3.82 (2H, s), 7.17 (2H, d, J=7.5 Hz), 7.33 (2H, d, J=7.5 Hz), 8.31 (3H, brs), 9.50 (1H, brs).

### Example 288

### N-[5-(aminomethyl)-6-isobutyl-2-methyl-4-(4-methylphenyl)pyridin-3-yl]propanamide dihydrochloride

N-[5-(Aminomethyl)-6-isobutyl-2-methyl-4-(4-methylphenyl)pyridin-3-yl]propanamide dihydrochloride (195 mg, yield 93%) was obtained as a white powder from tert-butyl {[5-amino-2-isobutyl-6-methyl-4-(4-methylphenyl)pyridin-3-yl]methyl}carbamate (192 mg, 0.5 mmol) and propionyl chloride (65 μL, 0.75 mmol) according to a method similar to the method of Example 223.

¹H-NMR (DMSO-d₆) δ:0.82 (3H, t, J=6.9 Hz), 0.98 (6H, d, J=6.6 Hz), 2.02 (2H, q, J=6.9 Hz), 2.08-2.32 (1H, m), 2.38 (3H, s), 2.55 (3H, s), 3.06 (2H, brs), 3.83 (2H, s), 7.17 (2H, d, J=7.8 Hz), 7.32 (2H, d, J=7.8 Hz), 8.37 (3H, brs), 9.49 (1H, brs).

### Example 289

### N-[5-(aminomethyl)-6-isobutyl-2-methyl-4-(4-methylphenyl)pyridin-3-yl]-2,2-dimethylpropanamide dihydrochloride

N-[5-(Aminomethyl)-6-isobutyl-2-methyl-4-(4-methylphenyl)pyridin-3-yl]-2,2-dimethylpropanamide dihydrochloride (184 mg, yield 72%) was obtained as a white powder from tert-butyl {[5-amino-2-isobutyl-6-methyl-4-(4-methylphenyl)pyridin-3-yl]methyl}carbamate (192 mg, 0.5 mmol) and pivaloyl chloride (92 μL, 0.75 mmol) according to a method similar to the method of Example 223.

¹H-NMR (DMSO-d₆) δ:0.89 (9H, s), 0.98 (6H, d, J=6.6 Hz), 2.12-2.24 (1H, m), 2.36 (3H, s), 2.51 (3H, s), 2.97 (2H, brs), 3.81 (2H, s), 7.14 (2H, d, J=8.1 Hz), 7.28 (2H, d, J=8.1 Hz), 8.28 (3H, brs), 8.95 (1H, brs).

### Example 290

### N-[5-(aminomethyl)-6-isobutyl-2-methyl-4-(4-methylphenyl)pyridin-3-yl]cyclopropanecarboxamide dihydrochloride

N-[5-(Aminomethyl)-6-isobutyl-2-methyl-4-(4-methylphenyl)pyridin-3-yl]cyclopropanecarboxamide dihydrochloride (170 mg, yield 85%) was obtained as a white powder from tert-butyl {[5-amino-2-isobutyl-6-methyl-4-(4-methylphenyl)pyridin-3-yl]methyl}carbamate (192 mg, 0.5 mmol) and cyclopropanecarbonyl chloride (68 μL, 0.75 mmol) according to a method similar to the method of Example 223.

¹H-NMR (DMSO-d₆) δ:0.58-0.67 (4H, m), 0.98 (6H, d, J=6.6 Hz), 1.51-1.58 (1H, m), 2.17-2.26 (1H, m), 2.39 (3H, s), 2.54 (3H, s), 3.02 (2H, brs), 3.81 (2H, s), 7.16 (2H, d, J=7.5 Hz), 7.32 (2H, d, J=7.5 Hz), 8.32 (3H, brs), 9.70 (1H, brs).

### Example 291

### N-[5-(aminomethyl)-6-isobutyl-2-methyl-4-(4-methylphenyl)pyridin-3-yl]cyclopentanecarboxamide dihydrochloride

N-[5-(Aminomethyl)-6-isobutyl-2-methyl-4-(4-methylphenyl)pyridin-3-yl]cyclopentanecarboxamide dihydrochloride (137 mg, yield 62%) was obtained as a white powder from tert-butyl {[5-amino-2-isobutyl-6-methyl-4-(4-methylphenyl)pyridin-3-yl]methyl}carbamate (192 mg, 0.5 mmol) and cyclopentanecarbonyl chloride (68 μL, 0.75 mmol) according to a method similar to the method of Example 223.

¹H-NMR (DMSO-d₆) δ: 0.98 (6H, d, J=6.6 Hz), 1.30-1.62 (9H, m), 2.15-2.24 (1H, m), 2.38 (3H, s), 2.50 (3H, s), 3.02 (2H, brs), 3.81 (2H, s), 7.15 (2H, d, J=7.8 Hz), 7.30 (2H, d, J=7.8 Hz), 8.32 (3H, brs), 9.39 (1H, brs).

### Example 292

### N-[5-(aminomethyl)-6-isobutyl-2-methyl-4-(4-methylphenyl)pyridin-3-yl]pyridine-2-carboxamide trihydrochloride

N-[5-(Aminomethyl)-6-isobutyl-2-methyl-4-(4-methylphenyl)pyridin-3-yl]pyridine-2-carboxamide trihydrochloride (218 mg, yield 91%) was obtained as a white powder from tert-butyl {[5-amino-2-isobutyl-6-methyl-4-(4-methylphenyl)pyridin-3-yl]methyl}carbamate (192 mg, 0.5 mmol) and pyridine-2-carbonyl chloride (106 mg, 0.75 mmol) according to a method similar to the method of Example 223.

¹H-NMR (DMSO-d₆) δ: 1.01 (6H, d, J=6.6 Hz), 2.20-2.28 (1H, m), 2.28 (3H, s), 2.64 (3H, s), 3.14 (2H, brs), 3.86 (2H, s), 7.20-7.27 (4H, m), 7.06-7.65 (1H, m), 7.94-8.02 (2H, m), 8.43 (3H, brs), 8.61 (1H, d, J=4.8 Hz), 10.33 (1H, s).

### Example 293

### N-[5-(aminomethyl)-6-isobutyl-2-methyl-4-(4-methylphenyl)pyridin-3-yl]nicotinamide trihydrochloride

N-[5-(Aminomethyl)-6-isobutyl-2-methyl-4-(4-methylphenyl)pyridin-3-yl]nicotinamide trihydrochloride (225 mg, yield 94%) was obtained as a white powder from tert-butyl {[5-amino-2-isobutyl-6-methyl-4-(4-methylphenyl)pyridin-3-yl]methyl}carbamate (192 mg, 0.5 mmol) and nicotinoyl chloride (106 mg, 0.75 mmol) according to a method similar to the method of Example 223.

¹H-NMR (DMSO-d₆) δ: 1.02 (6H, d, J=6.6 Hz), 2.23-2.31 (1H, m), 2.31 (3H, s), 2.73 (3H, s), 3.19 (2H, brs), 3.90 (2H, s), 7.28 (4H, s), 7.73-7.78 (1H, m), 8.35 (2H, d, J=8.1 Hz), 8.53 (3H, brs), 8.85 (1H, d, J=3.6 Hz), 8.94 (1H, s), 10.90 (1H, brs).

### Example 294

### N-[5-(aminomethyl)-6-isobutyl-2-methyl-4-(4-methylphenyl)pyridin-3-yl]isonicotinamide trihydrochloride

N-[5-(Aminomethyl)-6-isobutyl-2-methyl-4-(4-methylphenyl)pyridin-3-yl]isonicotinamide trihydrochloride (215 mg, yield 91%) was obtained as a white powder from tert-butyl {[5-amino-2-isobutyl-6-methyl-4-(4-methylphenyl)pyridin-3-yl]methyl}carbamate (192 mg, 0.5 mmol) and isonicotinoyl chloride (106 mg, 0.75 mmol) according to a method similar to the method of Example 223.

¹H-NMR (DMSO-d₆) δ: 1.01 (6H, d, J=6.6 Hz), 2.22-2.31 (1H, m), 2.31 (3H, s), 2.70 (3H, s), 3.51 (2H, brs), 3.88 (2H, s), 7.28 (4H, s), 7.87 (2H, d, J=6.0 Hz), 8.51 (3H, brs), 8.88 (2H, d, J=6.0 Hz), 11.20 (1H, brs).

### Example 295

### {[2-isobutyl-6-methyl-4-(4-methylphenyl)-5-(phenoxymethyl)pyridin-3-yl]methyl}amine dihydrochloride

1) tert-Butyl {[2-isobutyl-6-methyl-4-(4-methylphenyl)-5-(phenoxymethyl)pyridin-3-yl]methyl}carbamate (270 mg, yield 56%) was obtained as a colorless oil from tert-butyl {[5-(hydroxymethyl)-2-isobutyl-6-methyl-4-(4-methylphenyl)pyridin-3-yl]methyl}carbamate (0.40 g, 1.00 mmol) and phenol (94.5 mg, 1.00 mmol) according to a method similar to the method of Example 214-1).

¹H-NMR (CDCl₃) δ:0.99 (6H, d, J=6.6 Hz), 1.39 (9H, s), 2.18-2.27 (1H, m), 2.36 (3H, s), 2.63 (3H, s), 2.78 (2H, d, J=7.4 Hz), 4.10 (2H, d, J=5.7 Hz), 4.22 (1H, brs), 4.62 (2H, s), 6.78-6.82 (2H, m), 6.93 (1H, t, J=7.4 Hz), 7.05 (2H, d, J=8.1 Hz), 7.17 (2H, d, J=7.7 Hz), 7.21-7.24 (2H, m).

2) {[2-Isobutyl-6-methyl-4-(4-methylphenyl)-5-(phenoxymethyl)pyridin-3-yl]methyl}amine dihydrochloride (132 mg, yield 51%) was obtained as a colorless oil from tert-butyl {[2-isobutyl-6-methyl-4-(4-methylphenyl)-5-(phenoxymethyl)pyridin-3-yl]methyl}carbamate (0.27 g, 0.569 mmol) according to a method similar to the method of Example 2-3).

¹H-NMR (DMSO-d₆) δ:1.00 (6H, d, J=6.6 Hz), 2.17-2.26 (1H, m), 2.35 (3H, s), 2.82 (3H, brs), 3.12 (2H, brs), 3.83 (2H, d, J=4.9 Hz), 4.70 (2H, s), 6.85 (2H, d, J=7.9 Hz), 6.95 (1H, t, J=7.4 Hz), 7.23-7.33 (6H, m), 8.38 (3H, brs).

### Example 296

### 6-({[5-(aminomethyl)-6-isobutyl-2-methyl-4-(4-methylphenyl)pyridin-3-yl]methoxy}methyl)nicotinamide trihydrochloride

1) tert-Butyl {[5-({[5-(aminocarbonyl)pyridin-2-yl]methoxy}methyl)-2-isobutyl-6-methyl-4-(4-methylphenyl)pyridin-3-yl]methyl}carbamate (370 mg, yield 77%) was obtained as a white solid from 6-({[5-{[(tert-butoxycarbonyl)amino]methyl}-6-isobutyl-2-methyl-4-(4-methylphenyl)pyridin-3-yl]methoxy}methyl)nicotinic acid (0.48 g, 0.899 mmol) according to a method similar to the method of Example 3-1).

¹H-NMR (CDCl₃) δ:0.96 (6H, d, J=6.6 Hz), 1.38 (9H, s), 2.13-2.23 (1H, m), 2.40 (3H, s), 2.67 (3H, s), 2.78 (2H, d, J=7.4 Hz), 4.07 (2H, d, J=5.1 Hz), 4.23 (1H, brs), 4.27 (2H, s), 4.49 (2H, s), 7.03 (2H, d, J=7.9 Hz), 7.20 (2H, d, J=7.7 Hz), 7.38 (1H, d, J=7.9 Hz), 8.08 (1H, dd, J=8.1, 2.3 Hz), 8.90 (1H, d, J=2.3 Hz).

2) 6-({[5-(Aminomethyl)-6-isobutyl-2-methyl-4-(4-methylphenyl)pyridin-3-yl]methoxy}methyl)nicotinamide trihydrochloride (282 mg, yield 75%) was obtained as a white solid from tert-butyl {[5-({[5-(aminocarbonyl)pyridin-2-yl]methoxy}methyl)-2-isobutyl-6-methyl-4-(4-methylphenyl)pyridin-3-yl]methyl}carbamate (0.37 g, 0.695 mmol) according to a method similar to the method of Example 2-3).

¹H-NMR (DMSO-d₆) δ:0.99 (6H, d, J=6.6 Hz), 2.11-2.24 (1H, m), 2.39 (3H, s), 2.97 (3H, brs), 3.23 (2H, d, J=5.8 Hz), 3.82 (2H, d, J=5.3 Hz), 4.30 (2H, s), 4.52 (2H, s), 7.25 (2H, d, J=8.1 Hz), 7.32 (2H, d, J=8.1 Hz), 7.39-7.42 (1H, m), 7.61-7.69 (1H, m), 8.27-8.30 (1H, m), 8.50 (3H, brs), 8.99 (1H, brs).

### Example 297

### 4-{[5-(aminomethyl)-6-isobutyl-2-methyl-4-(4-methylphenyl)pyridin-3-yl]methoxy}isophthalic acid dihydrochloride

1) Dimethyl 4-{[5-{[(tert-butoxycarbonyl)amino]methyl}-6-isobutyl-2-methyl-4-(4-methylphenyl)pyridin-3-yl]methoxy}isophthalate (1.12 g, yield 75%) was obtained as a white solid from tert-butyl {[5-(hydroxymethyl)-2-isobutyl-6-methyl-4-(4-methylphenyl)pyridin-3-yl]methyl}carbamate (1.00 g, 2.51 mmol) and dimethyl 4-hydroxyisophthalate (528 mg, 2.51 mmol) according to a method similar to the method of Example 214-1).

¹H-NMR (CDCl₃) δ:0.99 (6H, d, J=6.8 Hz), 1.39 (9H, s), 2.19-2.31 (1H, m), 2.35 (3H, s), 2.66 (3H, s), 2.78 (2H, d, J=7.4 Hz), 3.83 (3H, s), 3.89 (3H, s), 4.06-4.11 (2H, m), 4.23 (1H, brs), 4.77 (2H, s), 6.71 (1H, d, J=8.9 Hz), 7.05 (2H, d, J=8.1 Hz), 7.16 (2H, d, J=7.9 Hz), 8.01 (1H, dd, J=8.7, 2.3 Hz), 8.41 (1H, d, J=2.3 Hz).

2) 4-{[5-{[(tert-Butoxycarbonyl)amino]methyl}-6-isobutyl-2-methyl-4-(4-methylphenyl)pyridin-3-yl]methoxy}isophthalic acid (310 mg, yield 90%) was obtained as a white solid from dimethyl 4-{[5-{[(tert-butoxycarbonyl)amino]methyl}-6-isobutyl-2-methyl-4-(4-methylphenyl)pyridin-3-yl]methoxy}isophthalate (0.36 g, 0.609 mmol) according to a method similar to the method of Example 9-1).

¹H-NMR (CDCl₃) δ:1.03 (6H, d, J=6.4 Hz), 1.37 (9H, s), 2.35 (3H, s), 2.96 (3H, brs), 3.13 (2H, brs), 4.16 (2H, brs), 4.94 (2H, brs), 6.76 (1H, brs), 7.07 (2H, brs), 7.22 (2H, d, J=7.7 Hz), 8.01 (1H, brs), 8.53 (1H, brs).

3) 4-{[5-(Aminomethyl)-6-isobutyl-2-methyl-4-(4-methylphenyl)pyridin-3-yl]methoxy}isophthalic acid dihydrochloride (256 mg, yield 86%) was obtained as a white solid from 4-{[5-{[(tert-butoxycarbonyl)amino]methyl}-6-isobutyl-2-methyl-4-(4-methylphenyl)pyridin-3-yl]methoxy}isophthalic acid (0.31 g, 0.551 mmol) according to a method similar to the method of Example 2-3).

¹H-NMR (DMSO-d₆) δ:1.00 (6H, d, J=6.6 Hz), 2.16-2.28 (1H, m), 2.35 (3H, s), 2.85 (3H, brs), 3.08 (2H, brs), 3.83 (2H, brs), 4.86 (2H, s), 7.01 (1H, d, J=8.9 Hz), 7.27 (2H, d, J=8.1 Hz), 7.31 (2H, d, J=7.7 Hz), 7.97 (1H, dd, J=8.7, 2.3 Hz), 8.18 (1H, d, J=2.1 Hz), 8.34 (3H, brs).

### Example 298

### methyl 2-{(E)-2-[5-(aminomethyl)-6-isobutyl-2-methyl-4-(4-methylphenyl)pyridin-3-yl]vinyl}benzoate dihydrochloride

Methyl 2-{(E)-2-[5-(aminomethyl)-6-isobutyl-2-methyl-4-(4-methylphenyl)pyridin-3-yl]vinyl}benzoate dihydrochloride (31.4 mg, yield 33%) was obtained as a white solid from methyl 2-{(E)-2-[5-{[(tert-butoxycarbonyl)amino]methyl}-6-isobutyl-2-methyl-4-(4-methylphenyl)pyridin-3-yl]vinyl}benzoate (0.10 g, 0.189 mmol) according to a method similar to the method of Example 2-3).

¹H-NMR (DMSO-d₆) δ:1.01 (6H, d, J=6.4 Hz), 2.16-2.28 (1H, m), 2.38 (3H, s), 2.86 (3H, brs), 3.06 (2H, brs), 3.83-3.88 (5H, m), 6.53 (1H, d, J=16.8 Hz), 7.17 (1H, d, J=16.8 Hz), 7.24 (2H, d, J=7.7 Hz), 7.29 (1H, d, J=7.7 Hz), 7.35 (2H, d, J=7.9 Hz), 7.40 (1H, t, J=7.5 Hz), 7.53 (1H, t, J=7.5 Hz), 7.79 (1H, dd, J=7.8, 1.2 Hz), 8.32 (3H, brs).

### Example 299

### 4-[1-({[5-(aminomethyl)-6-isobutyl-2-methyl-4-(4-methylphenyl)pyridin-3-yl]carbonyl}oxy)ethyl]benzoic acid dihydrochloride

1) 1-[4-(Methoxycarbonyl)phenyl]ethyl 5-{[(tert-butoxycarbonyl)amino]methyl}-6-isobutyl-2-methyl-4-(4-methylphenyl)nicotinate (1.02 g, yield 73%) was obtained as a colorless oil from 5-{[(tert-butoxycarbonyl)amino]methyl}-6-isobutyl-2-methyl-4-(4-methylphenyl)nicotinic acid (1.00 g, 2.42 mmol) and methyl 4-(1-hydroxyethyl)benzoate (486 mg, 2.42 mmol) according to a method similar to the method of Example 247-1).

¹H-NMR (CDCl₃) δ:0.97 (6H, d, J=6.6 Hz), 1.25 (3H, d, J=7.0 Hz), 1.39 (9H, s), 2.16-2.24 (1H, m), 2.33 (3H, s), 2.48 (3H, s), 2.78 (2H, d, J=7.4 Hz), 3.92 (3H, s), 4.11-4.16 (2H, m), 4.22 (1H, brs), 5.73-5.79 (1H, m), 6.96-6.99 (1H, m), 7.04-7.09 (2H, m), 7.13-7.17 (3H, m), 7.93 (2H, d, J=8.3 Hz).

2) 4-[1-({[5-{[(tert-Butoxycarbonyl)amino]methyl}-6-isobutyl-2-methyl-4-(4-methylphenyl)pyridin-3-yl]carbonyl}oxy)ethyl]benzoic acid (950 mg, yield 95%) was obtained as a colorless oil from 1-[4-(methoxycarbonyl)phenyl]ethyl 5-{[(tert-butoxycarbonyl)amino]methyl}-6-isobutyl-2-methyl-4-(4-methylphenyl)nicotinate (1.02 g, 1.77 mmol) according to a method similar to the method of Example 9-1).

¹H-NMR (CDCl₃) δ:0.97 (6H, d, J=6.8 Hz), 1.26 (3H, d, J=6.8 Hz), 1.39 (9H, s), 2.15-2.26 (1H, m), 2.34 (3H, s), 2.50 (3H, s), 2.79 (2H, d, J=7.2 Hz), 4.11-4.16 (2H, m), 4.24 (1H, brs), 5.79 (1H, q, J=6.6 Hz), 7.00-7.13 (4H, m), 7.18 (2H, d, J=8.1 Hz), 7.99 (2H, d, J=8.3 Hz).

3) 4-[1-({[5-(Aminomethyl)-6-isobutyl-2-methyl-4-(4-methylphenyl)pyridin-3-yl]carbonyl}oxy)ethyl]benzoic acid dihydrochloride (259 mg, yield 93%) was obtained as a white solid from 4-[1-({[5-{[(tert-butoxycarbonyl)amino]methyl}-6-isobutyl-2-methyl-4-(4-methylphenyl)pyridin-3-yl]carbonyl}oxy)ethyl]benzoic acid (0.30 g, 0.522 mmol) according to a method similar to the method of Example 2-3).

¹H-NMR (DMSO-d₆) δ:0.97 (6H, d, J=6.8 Hz), 1.22 (3H, d, J=6.6 Hz), 2.17-2.26 (1H, m), 2.33 (3H, s), 2.47 (3H, brs), 2.88 (2H, d, J=5.7 Hz), 3.81 (2H, d, J=5.5 Hz), 5.76 (1H, q, J=6.6 Hz), 7.11-7.25 (6H, m), 8.27 (3H, brs).

### Example 300

### [(2-isobutyl-6-methyl-4-(4-methylphenyl)-5-{[2-(methylthio)phenoxy]methyl}pyridin-3-yl)methyl]amine dihydrochloride

1) tert-Butyl [(2-isobutyl-6-methyl-4-(4-methylphenyl)-5-{[2-(methylthio)phenoxy]methyl}pyridin-3-yl)methyl]carbamate (1.37 g, yield 70%) was obtained as a colorless oil from tert-butyl {[5-(hydroxymethyl)-2-isobutyl-6-methyl-4-(4-methylphenyl)pyridin-3-yl]methyl}carbamate (1.50 g, 3.76 mmol) and 2-(methylthio)phenol (573 mg, 3.76 mmol) according to a method similar to the method of Example 214-1).

¹H-NMR (CDCl₃) δ:0.99 (6H, d, J=6.6 Hz), 1.39 (9H, s), 2.19-2.31 (1H, m), 2.36 (3H, s), 2.37 (3H, s), 2.69 (3H, s), 2.78 (2H, d, J=7.4 Hz), 4.09-4.11 (2H, m), 4.21 (1H, brs), 4.68 (2H, s), 6.57 (1H, dd, J=7.9, 1.3 Hz), 6.91-7.04 (2H, m), 7.06-7.12 (3H, m), 7.17 (2H, d, J=7.7 Hz).

2) [(2-Isobutyl-6-methyl-4-(4-methylphenyl)-5-{[2-(methylthio)phenoxy]methyl}pyridin-3-yl)methyl]amine dihydrochloride (112 mg, yield 69%) was obtained as a white solid from tert-butyl [(2-isobutyl-6-methyl-4-(4-methylphenyl)-5-{[2-(methylthio)phenoxy]methyl}pyridin-3-yl)methyl]carbamate (0.17 mg, 0.326 mmol) according to a method similar to the method of Example 2-3).

¹H-NMR (DMSO-d₆) δ:1.00 (6H, d, J=6.6 Hz), 2.18-2.27 (1H, m), 2.35 (3H, s), 2.36 (3H, s), 2.88 (2H, brs), 3.15 (2H, brs), 3.83 (2H, brs), 4.75 (2H, s), 6.57 (1H, d, J=6.8 Hz), 6.96-7.07 (2H, m), 7.13-7.16 (1H, m), 7.28 (2H, d, J=8.3 Hz), 7.32 (2H, d, J=7.4 Hz), 8.41 (3H, brs).

### Example 301

### [(2-isobutyl-6-methyl-4-(4-methylphenyl)-5-{[2-(methylsulfonyl)phenoxy]methyl}pyridin-3-yl)methyl]amine dihydrochloride

1) tert-Butyl [(2-isobutyl-6-methyl-4-(4-methylphenyl)-5-{[2-(methylsulfonyl)phenoxy]methyl}pyridin-3-yl)methyl]carbamate (330 mg, yield 81%) was obtained as a white solid from tert-butyl [(2-isobutyl-6-methyl-4-(4-methylphenyl)-5-{[2-(methylthio)phenoxy]methyl}pyridin-3-yl)methyl]carbamate (0.38 g, 0.730 mmol) according to a method similar to the method of Example 91-1).

¹H-NMR (CDCl₃) δ:0.99 (6H, d, J=6.8 Hz), 1.39 (9H, s), 2.21-2.30 (1H, m), 2.35 (3H, s), 2.67 (3H, s), 2.79 (2H, d, J=7.4 Hz), 3.08 (3H, s), 4.11 (2H, d, J=5.1 Hz), 4.27 (1H, brs), 4.79 (2H, s), 6.76 (1H, d, J=8.1 Hz), 7.06-7.10 (3H, m), 7.18 (2H, d, J=7.9 Hz), 7.45-7.50 (1H, m), 7.97 (1H, dd, J=7.7, 1.7 Hz).

2) [(2-Isobutyl-6-methyl-4-(4-methylphenyl)-5-{[2-(methylsulfonyl)phenoxy]methyl}pyridin-3-yl)methyl]amine dihydrochloride (227 mg, yield 59%) was obtained as a white solid from tert-butyl [(2-isobutyl-6-methyl-4-(4-methylphenyl)-5-{[2-(methylsulfonyl)phenoxy]methyl}pyridin-3-yl)methyl]carbamate (0.33 g, 0.597 mmol) according to a method similar to the method of Example 2-3).

¹H-NMR (DMSO-d₆) δ:1.00 (6H, d, J=6.4 Hz), 2.17-2.28 (1H, m), 2.35 (3H, s), 2.84 (3H, brs), 3.05-3.17 (5H, m), 3.84 (2H, d, J=4.7 Hz), 4.87 (2H, s), 7.11 (1H, d, J=8.3 Hz), 7.18 (1H, t, J=7.6 Hz), 7.28-7.33 (4H, m), 7.60-7.66 (1H, m), 7.81 (1H, dd, J=7.7, 1.7 Hz), 8.40 (3H, brs).

### Example 302

### [(2-isobutyl-6-methyl-4-(4-methylphenyl-5-{[2-(methylsulfinyl)phenoxy]methyl}pyridin-3-yl)methyl]amine dihydrochloride

1) To a mixed solution of tert-butyl [(2-isobutyl-6-methyl-4-(4-methylphenyl)-5-{[2-(methylthio)phenoxy]methyl}pyridin-3-yl)methyl]carbamate (0.47 g, 0.902 mmol) in methanol (10 mL) and water (10 mL) was added sodium periodate (377 mg, 1.76 mmol) and the mixture was stirred at room temperature for 2 days. The reaction mixture was diluted with ethyl acetate, washed successively with water and saturated brine and dried over anhydrous magnesium sulfate. The solvent was evaporated under reduced pressure and the obtained residue was purified by silica gel column chromatography to give tert-butyl [(2-isobutyl-6-methyl-4-(4-methylphenyl)-5-{[2-(methylsulfinyl)phenoxy]methyl}pyridin-3-yl)methyl]carbamate (164 mg, yield 33%) as a yellow oil.

¹H-NMR (CDCl₃) δ:1.00 (6H, d, J=6.6 Hz), 1.39 (9H, s), 2.21-2.29 (1H, m), 2.35 (3H, s), 2.61 (3H, s), 2.69 (3H, s), 2.80 (2H, d, J=7.4 Hz), 4.09-4.11 (2H, m), 4.23 (1H, brs), 4.59 (1H, d, J=10.0 Hz), 4.83 (1H, d, J=10.0 Hz), 6.71 (1H, d, J=8.1 Hz), 6.95-6.98 (1H, m), 7.02-7.05 (1H, m), 7.16-7.21 (3H, m), 7.32-7.38 (1H, m), 7.82 (1H, dd, J=7.7, 1.7 Hz).

2) [(2-Isobutyl-6-methyl-4-(4-methylphenyl)-5-{[2-(methylsulfinyl)phenoxy]methyl}pyridin-3-yl)methyl]amine dihydrochloride (97.4 mg, yield 62%) was obtained as a white solid from tert-butyl [(2-isobutyl-6-methyl-4-(4-methylphenyl)-5-{[2-(methylsulfinyl)phenoxy]methyl}pyridin-3-yl)methyl]carbamate (164 mg, 0.306 mmol) according to a method similar to the method of Example 2-3).

¹H-NMR (DMSO-d₆) δ:1.00 (6H, d, J=6.6 Hz), 2.17-2.27 (1H, m), 2.34 (3H, s), 2.63 (3H, s), 2.77 (3H, brs), 3.06 (2H, brs), 3.82 (2H, brs), 4.70 (1H, d, J=10.6 Hz), 4.90 (1H, d, J=10.7 Hz), 6.99 (1H, d, J=8.1 Hz), 7.20-7.33 (5H, m), 7.42-7.47 (1H, m), 7.64 (1H, dd, J=7.5, 1.7 Hz), 8.31 (3H, brs).

### Example 303

### 3-{[5-(aminomethyl)-6-isobutyl-2-methyl-4-(4-methylphenyl)pyridin-3-yl]methoxy}-2-naphthamide dihydrochloride

1) tert-Butyl {[5-({[3-(aminocarbonyl)-2-naphthyl]oxy}methyl)-2-isobutyl-6-methyl-4-(4-methylphenyl)pyridin-3-yl]methyl}carbamate (230 mg, yield 46%) was obtained as a white powder from 3-{[5-{[(tert-butoxycarbonyl)amino]methyl}-6-isobutyl-2-methyl-4-(4-methylphenyl)pyridin-3-yl]methoxy}-2-naphthoic acid (500 mg, 0.879 mmol) according to a method similar to the method of Example 3-1).

¹H-NMR (CDCl₃) δ:0.89 (6H, d, J=6.6 Hz), 1.35 (9H, s), 2.07-2.22 (1H, m), 2.28 (3H, s), 2.79 (3H, s), 2.87 (2H, d, J=7.2 Hz), 4.14-4.21 (3H, m), 4.95 (2H, s), 7.04 (1H, s), 7.08-7.21 (4H, m), 7.42-7.52 (1H, m), 7.63 (1H, d, J=7.5 Hz), 7.74 (1H, d, J=7.5 Hz), 7.81 (1H, d, J=8.1 Hz), 8.67 (1H, s), 11.73 (2H, s).

2) 3-{[5-(Aminomethyl)-6-isobutyl-2-methyl-4-(4-methylphenyl)pyridin-3-yl]methoxy}-2-naphthamide dihydrochloride (200 mg, yield 91%) was obtained as a white powder from tert-butyl {[5-({[3-(aminocarbonyl)-2-naphthyl]oxy}methyl)-2-isobutyl-6-methyl-4-(4-methylphenyl)pyridin-3-yl]methyl}carbamate (230 mg, 0.405 mmol) according to a method similar to the method of Example 2-3).

¹H-NMR (DMSO-d₆)δ:1.00 (6H, d, J=6.4 Hz), 2.17-2.30 (1H, m), 2.32 (3H, s), 2.51 (3H, s), 2.81 (2H, s), 3.83 (2H, s), 4.88 (2H, s), 7.25-7.33 (4H, m), 7.40 (1H, t, J=7.5 Hz), 7.50 (1H, t, J=7.5 Hz), 7.75 (1H, d, J=8.1 Hz), 7.92 (1H, d, J=7.9 Hz), 8.12 (1H, s), 8.42 (1H, s), 8.62 (3H, brs).

### Example 304

### 5-(aminomethyl)-6-isobutyl-2-methyl-4-(4-methylphenyl)-N-phenylnicotinamide

To a solution of 5-({[(benzyloxy)carbonyl]amino}methyl)-6-isobutyl-2-methyl-4-(4-methylphenyl)nicotinic acid (523 mg, 1.17 mmol) in tetrahydrofuran (5 mL) was added oxalyl chloride (120 μL, 1.4 mmol) and one drop of N,N-dimethylformamide was added. The reaction solution was stirred for 3 hrs. and the reaction mixture was concentrated. The residue was dissolved in tetrahydrofuran (5 mL). Aniline (91 μL, 1.0 mmol) and triethylamine (210 μL, 1.5 mmol) were added and the mixture was stirred for 30 min. Water was added to the reaction mixture and the mixture was extracted with ethyl acetate. The organic layer was washed with saturated brine and dried over anhydrous magnesium sulfate. The solvent was evaporated under reduced pressure and the obtained residue was purified by silica gel column chromatography to give an oil. To a solution of the oil in ethanol (5 mL) was added 10% palladium—carbon (50 mg) and the mixture was stirred under a hydrogen atmosphere at room temperature for 3 hrs. The reaction mixture was filtered and the filtrate was concentrated. The obtained oil was crystallized from hexane and diethyl ether to give 5-(aminomethyl)-6-isobutyl-2-methyl-4-(4-methylphenyl)-N-phenylnicotinamide (320 mg, yield 83%) as a white powder.

¹H-NMR (CDCl₃) δ: 1.00 (6H, d, J=6.6 Hz), 2.17-2.31 (1H, m), 2.34 (3H, s), 2.65 (3H, s), 2.82 (2H, d, J=7.5 Hz), 3.69 (2H, s), 6.93 (1H, brs), 7.04-7.26 (9H, m).

### Example 305

### methyl 3-{[5-(aminomethyl)-6-isobutyl-2-methyl-4-(4-methylphenyl)pyridin-3-yl]methoxy}-1-methyl-1H-pyrazole-4-carboxylate dihydrochloride

1) Ethyl 3-{[5-{[(tert-butoxycarbonyl)amino]methyl}-6-isobutyl-2-methyl-4-(4-methylphenyl)pyridin-3-yl]methoxy}-1-methyl-1H-pyrazole-4-carboxylate (3.23 g, yield 79%) was obtained as a colorless oil from tert-butyl {[5-(hydroxymethyl)-2-isobutyl-6-methyl-4-(4-methylphenyl)pyridin-3-yl]methyl}carbamate (3.00 g, 7.52 mmol) and ethyl 3-hydroxy-1-methyl-1H-pyrazole-4-carboxylate (1.28 g, 7.52 mmol) according to a method similar to the method of Example 183-1).

¹H-NMR (CDCl₃) δ:0.98 (6H, d, J=6.8 Hz), 1.28 (3H, t, J=7.1 Hz), 1.39 (9H, s), 2.17-2.26 (1H, m), 2.36 (3H, s), 2.66 (3H, s), 2.77 (2H, d, J=7.4 Hz), 3.67 (3H, s), 4.08 (2H, d, J=4.7 Hz), 4.19-4.26 (3H, m), 4.90 (2H, s), 7.10 (2H, d, J=8.1 Hz), 7.16 (2H, d, J=8.1 Hz), 7.61 (1H, s).

2) 3-{[5-{[(tert-Butoxycarbonyl)amino]methyl}-6-isobutyl-2-methyl-4-(4-methylphenyl)pyridin-3-yl]methoxy}-1-methyl-1H-pyrazole-4-carboxylic acid (1.58 g, yield 51%) was obtained as a white solid from ethyl 3-{[5-{[(tert-butoxycarbonyl)amino]methyl}-6-isobutyl-2-methyl-4-(4-methylphenyl)pyridin-3-yl]methoxy}-1-methyl-1H-pyrazole-4-carboxylate (3.23 g, 5.86 mmol) according to a method similar to the method of Example 9-1).

¹H-NMR (CDCl₃) δ:0.99 (6H, d, J=6.8 Hz), 1.38 (9H, s), 2.15-2.28 (1H, m), 2.36 (3H, s), 2.66 (3H, s), 2.79 (2H, d, J=7.4 Hz), 3.71 (3H, s), 4.04-4.09 (2H, m), 4.23 (1H, brs), 4.98 (2H, s), 7.05 (2H, d, J=8.1 Hz), 7.19 (2H, d, J=7.7 Hz), 7.69 (1H, s).

3) 3-{[5-{[(tert-Butoxycarbonyl)amino]methyl}-6-isobutyl-2-methyl-4-(4-methylphenyl)pyridin-3-yl]methoxy}-1-methyl-1H-pyrazole-4-carboxylic acid (0.50 g, 0.957 mmol) was dissolved in N,N-dimethylformamide (5 mL) and methyl iodide (176 mg, 1.24 mmol) and potassium carbonate (0.20 g, 1.44 mmol) were added. The mixture was stirred at room temperature for 1 hr. Ethyl acetate was added to the reaction mixture, and the mixture was washed with saturated brine and dried over anhydrous magnesium sulfate. The solvent was evaporated under reduced pressure and the residue was purified by silica gel column chromatography to give methyl 3-{[5-{[( tert-butoxycarbonyl)amino]methyl}-6-isobutyl-2-methyl-4-(4-methylphenyl)pyridin-3-yl]methoxy}-1-methyl-1H-pyrazole-4-carboxylate (470 mg, yield 91%) as a white solid.

¹H-NMR (CDCl₃) δ:0.99 (6H, d, J=6.6 Hz), 1.39 (9H, s), 2.17-2.26 (1H, m), 2.36 (3H, s), 2.66 (3H, s), 2.77 (2H, d, J=7.4 Hz), 3.68 (3H, s), 3.76 (3H, s), 4.08 (2H, d, J=4.7 Hz), 4.23 (1H, brs), 4.90 (2H, s), 7.10 (2H, d, J=7.9 Hz), 7.16 (2H, d, J=7.9 Hz), 7.62 (1H, s).

4) Methyl 3-{[5-(aminomethyl)-6-isobutyl-2-methyl-4-(4-methylphenyl)pyridin-3-yl]methoxy}-1-methyl-1H-pyrazole-4-carboxylate dihydrochloride (382 mg, yield 85%) was obtained as a white solid from methyl 3-{[5-{[(tert-butoxycarbonyl)amino]methyl}-6-isobutyl-2-methyl-4-(4-methylphenyl)pyridin-3-yl]methoxy}-1-methyl-1H-pyrazole-4-carboxylate (0.47 g, 0.876 mmol) according to a method similar to the method of Example 2-3).

¹H-NMR (DMSO-d₆) δ:1.00 (6H, d, J=6.6 Hz), 2.14-2.28 (1H, m), 2.38 (3H, s), 2.90 (3H, brs), 3.16 (2H, brs), 3.65 (3H, s), 3.66 (3H, s), 3.82 (2H, d, J=5.1 Hz), 4.90 (2H, s), 7.27 (2H, d, J=8.1 Hz), 7.33 (2H, d, J=8.1 Hz), 8.09 (1H, s), 8.41 (3H, brs).

### Example 306

### 3-{[5-(aminomethyl)-6-isobutyl-2-methyl-4-(4-methylphenyl)pyridin-3-yl]methoxy}-1-methyl-1H-pyrazole-4-carboxylic acid dihydrochloride

3-{[5-(Aminomethyl)-6-isobutyl-2-methyl-4-(4-methylphenyl)pyridin-3-yl]methoxy}-1-methyl-1H-pyrazole-4-carboxylic acid dihydrochloride (268 mg, yield 94%) was obtained as a white solid from 3-{[5-{[(tert-butoxycarbonyl)amino]methyl}-6-isobutyl-2-methyl-4-(4-methylphenyl)pyridin-3-yl]methoxy}-1-methyl-1H-pyrazole-4-carboxylic acid (0.30 g, 0.574 mmol) according to a method similar to the method of Example 2-3).

¹H-NMR (DMSO-d₆) δ:0.99 (6H, d, J=6.4 Hz), 2.14-2.25 (1H, m), 2.39 (3H, s), 2.88 (3H, brs), 3.14 (2H, brs), 3.64 (3H, s), 3.82 (2H, d, J=4.7 Hz), 4.87 (2H, s), 7.28 (2H, d, J=7.9 Hz), 7.34 (2H, d, J=8.1 Hz), 8.00 (1H, s), 8.38 (3H, brs).

### Example 307

### 3-{[5-(aminomethyl)-6-isobutyl-2-methyl-4-(4-methylphenyl)pyridin-3-yl]methoxy}-1-methyl-1H-pyrazole-4-carboxamide dihydrochloride

1) tert-Butyl {[5-({[4-(aminocarbonyl)-1-methyl-1H-pyrazol-3-yl]oxy}methyl)-2-isobutyl-6-methyl-4-(4-methylphenyl)pyridin-3-yl]methyl}carbamate (307 mg, yield 61%) was obtained as a colorless oil from 3-{[5-{[(tert-butoxycarbonyl)amino]methyl}-6-isobutyl-2-methyl-4-(4-methylphenyl)pyridin-3-yl]methoxy}-1-methyl-1H-pyrazole-4-carboxylic acid (0.50 g, 0.957 mmol) according to a method similar to the method of Example 3-1).

¹H-NMR (CDCl₃) δ:0.99 (6H, d, J=6.6 Hz), 1.39 (9H, s), 2.19-2.28 (1H, m), 2.37 (3H, s), 2.65 (3H, s), 2.79 (2H, d, J=7.4 Hz), 3.69 (3H, s), 4.09 (2H, d, J=4.9 Hz), 4.22 (1H, brs), 4.98 (2H, s), 5.30 (1H, brs), 6.43 (1H, brs), 7.01 (2H, d, J=8.1 Hz), 7.20 (2H, d, J=7.7 Hz), 7.69 (1H, s).

2) 3-{[5-(Aminomethyl)-6-isobutyl-2-methyl-4-(4-methylphenyl)pyridin-3-yl]methoxy}-1-methyl-1H-pyrazole-4-carboxamide dihydrochloride (253 mg, yield 87%) was obtained as a white solid from tert-butyl {[5-({[4-(aminocarbonyl)-1-methyl-1H-pyrazol-3-yl]oxy}methyl)-2-isobutyl-6-methyl-4-(4-methylphenyl)pyridin-3-yl]methyl}carbamate (307 mg, 0.588 mmol) according to a method similar to the method of Example 2-3).

¹H-NMR (DMSO-d₆) δ:1.00 (6H, d, J=6.6 Hz), 2.14-2.27 (1H, m), 2.38 (3H, s), 2.93 (3H, brs), 3.17 (2H, brs), 3.63 (3H, s), 3.82 (2H, d, J=4.7 Hz), 4.93 (2H, s), 6.37 (1H, brs), 7.08 (1H, brs), 7.29 (2H, d, J=7.9 Hz), 7.35 (2H, d, J=8.1 Hz), 7.91 (1H, s), 8.42 (3H, brs).

### Example 308

### (3-{[5-(aminomethyl)-6-isobutyl-2-methyl-4-(4-methylphenyl)pyridin-3-yl]methoxy}-1-methyl-1H-pyrazol-4-yl)acetic acid dihydrochloride

1) To a solution of tert-butyl {[5-(hydroxymethyl)-2-isobutyl-6-methyl-4-(4-methylphenyl)pyridin-3-yl]methyl}carbamate (1.00 g, 2.51 mmol), methyl (3-hydroxy-1-methyl-1H-pyrazol-4-yl)acetate (0.43 g, 2.51 mmol) and tributylphosphine (0.61 g, 3.01 mmol) in tetrahydrofuran (20 mL) was added 1,1′-(azodicarbonyl)dipiperidine (0.76 g, 3.01 mmol) and the mixture was stirred at room temperature for 30 min. The reaction mixture was filtered and the solvent in the filtrate was evaporated under reduced pressure. The obtained residue was purified by silica gel column chromatography to give methyl (3-{[5-{[(tert-butoxycarbonyl)amino]methyl}-6-isobutyl-2-methyl-4-(4-methylphenyl)pyridin-3-yl]methoxy}-1-methyl-1H-pyrazol-4-yl)acetate (1.20 g, yield 86%) as a colorless oil. Then, (3-{[5-{[(tert-butoxycarbonyl)amino]methyl}-6-isobutyl-2-methyl-4-(4-methylphenyl)pyridin-3-yl]methoxy}-1-methyl-1H-pyrazol-4-yl)acetic acid (173 mg, yield 15%) was obtained as a white solid from methyl (3-{[5-{[(tert-butoxycarbonyl)amino]methyl}-6-isobutyl-2-methyl-4-(4-methylphenyl)pyridin-3-yl]methoxy}-1-methyl-1H-pyrazol-4-yl)acetate (1.20 g, 2.18 mmol) according to a method similar to the method of Example 9-1).

¹H-NMR (CDCl₃) δ:0.95 (6H, d, J=6.6 Hz), 1.38 (9H, s), 2.12-2.30 (1H, m), 2.36 (3H, s), 2.62 (3H, s), 2.80 (2H, d, J=7.2 Hz), 3.35 (2H, s), 3.66 (3H, s), 4.05-4.09 (2H, m), 4.27 (1H, brs), 4.84 (2H, s), 7.03 (2H, d, J=7.9 Hz), 7.12 (1H, s), 7.18 (2H, d, J=7.7 Hz).

2) (3-{[5-(Aminomethyl)-6-isobutyl-2-methyl-4-(4-methylphenyl)pyridin-3-yl]methoxy}-1-methyl-1H-pyrazol-4-yl)acetic acid dihydrochloride (84.2 mg, yield 51%) was obtained as a white solid from (3-{[5-{[(tert-butoxycarbonyl)amino]methyl}-6-isobutyl-2-methyl-4-(4-methylphenyl)pyridin-3-yl]methoxy}-1-methyl-1H-pyrazol-4-yl)acetic acid (173 mg, 0.323 mmol) according to a method similar to the method of Example 2-3).

¹H-NMR (DMSO-d₆) δ:0.98 (6H, d, J=6.6 Hz), 2.16-2.27 (1H, m), 2.38 (3H, s), 2.76 (3H, brs), 3.00 (2H, brs), 3.15 (2H, s), 3.58 (3H, s), 3.77-3.84 (2H, m), 4.76 (2H, s), 7.23 (2H, d, J=7.7 Hz), 7.33 (2H, d, J=7.5 Hz), 7.37 (1H, s), 8.18 (3H, brs).

### Example 309

### N-[5-(aminomethyl)-6-isobutyl-2-methyl-4-(4-methylphenyl)pyridin-3-yl]-3-(1H-tetrazol-5-yl)benzamide dihydrochloride

To a solution of tert-butyl {[5-amino-2-isobutyl-6-methyl-4-(4-methylphenyl)pyridin-3-yl]methyl}carbamate (383 mg, 1.0 mmol) in tetrahydrofuran (5 mL) was added 3-cyanobenzoyl chloride (245 mg, 1.5 mmol) and triethylamine (280 μL, 2.0 mmol) was added. The mixture was stirred for 18 hrs. Saturated aqueous sodium hydrogen carbonate solution (5 mL) was added to the reaction mixture and the mixture was extracted with ethyl acetate. The organic layer was washed with saturated brine and dried over anhydrous magnesium sulfate. The solvent was evaporated under reduced pressure and the obtained residue was purified by silica gel column chromatography to give an oil. To a solution of the obtained oil in dimethyl sulfoxide (3 mL) were added sodium azide (97 mg, 1.5 mmol) and ammonium chloride (312 mg, 2.0 mmol) and the mixture was stirred at 100° C. for 3 hrs. Distilled water (10 mL) was added to the reaction mixture and the mixture was extracted with ethyl acetate. The organic layer was washed with saturated brine and dried over anhydrous magnesium sulfate. The solvent was evaporated under reduced pressure and the obtained residue was purified by silica gel column chromatography to give an oil. To a solution of the obtained oil in ethyl acetate (2 mL) was added 4N hydrogen chloride ethyl acetate solution (2 mL) and the resulting mixture was stirred at room temperature for 3 hrs. The solvent was evaporated under reduced pressure and the obtained residue was crystallized from hexane to give N-[5-(aminomethyl)-6-isobutyl-2-methyl-4-(4-methylphenyl)pyridin-3-yl]-3-(1H-tetrazol-5-yl)benzamide dihydrochloride (86 mg, yield 16%) as a white powder.

¹H-NMR (DOSO-d₆)δ:0.99 (6H, d, J=6.6 Hz), 2.11-2.27 (1H, m), 2.27 (3H, s), 2.52 (3H, s), 2.93 (2H, s), 3.83 (2H, s), 7.22 (4H, s), 7.64 (1H, t, J=7.8 Hz), 7.76 (1H, d, J=7.8 Hz), 8.16 (4H, brs), 8.34 (1H, brs), 10.10 (1H, brs).

### Example 310

### methyl 2-{[5-(aminomethyl)-6-isobutyl-2-methyl-4-(4-methylphenyl)pyridin-3-yl]methoxy}-3-methylbenzoate dihydrochloride

1) Methyl 2-{[5-{[(tert-butoxycarbonyl)amino]methyl}-6-isobutyl-2-methyl-4-(4-methylphenyl)pyridin-3-yl]methoxy}-3-methylbenzoate (600 mg, yield 44%) was obtained as a white powder from tert-butyl {[5-(hydroxymethyl)-2-isobutyl-6-methyl-4-(4-methylphenyl)pyridin-3-yl]methyl}carbamate (1.0 g, 2.51 mmol) and methyl 2-hydroxy-3-methylbenzoate (500 mg, 3.01 mmol) according to a method similar to the method of Example 214-1).

¹H-NMR (CDCl₃) δ:0.98 (6H, d, J=6.6 Hz), 1.38 (9H, s), 1.80 (3H, s), 2.15-2.28 (1H, m), 2.34 (3H, s), 2.70 (3H, s), 2.77 (2H, d, J=7.4 Hz), 3.66 (3H, s), 3.97 (2H, d, J=4.9 Hz), 4.20 (1H, brs), 4.76 (2H, s), 6.52 (2H, d, J=7.9 Hz), 6.99 (2H, d, J=7.9 Hz), 7.01-7.06 (1H, m), 7.19 (1H, dd, J=7.4, 1.0 Hz), 7.44 (1H, dd, J=7.7, 1.0 Hz).

2) Methyl 2-{[5-(aminomethyl)-6-isobutyl-2-methyl-4-(4-methylphenyl)pyridin-3-yl]methoxy}-3-methylbenzoate dihydrochloride (215 mg, yield 94%) was obtained as a white powder from methyl 2-{[5-{[(tert-butoxycarbonyl)amino]methyl}-6-isobutyl-2-methyl-4-(4-methylphenyl)pyridin-3-yl]methoxy}-3-methylbenzoate (240 mg, 0.439 mmol) according to a method similar to the method of Example 2-3).

¹H-NMR (DMSO-d₆)δ:1.01 (6H, d, J=6.4 Hz), 1.82 (3H, s), 2.14-2.29 (1H, m), 2.36 (3H, s), 3.02 (3H, s), 3.31 (2H, d, J=6.8 Hz), 3.67 (3H, s), 3.78 (2H, d, J=2.45 Hz), 4.81 (2H, s), 6.89 (2H, d, J=7.7 Hz), 7.11-7.20 (3H, m), 7.33 (1H, d, J=7.0 Hz), 7.43 (1H, d, J=7.0 Hz), 8.63 (3H, brs).

### Example 311

### 2-[5-(aminomethyl)-6-isobutyl-2-methyl-4-(4-methylphenyl)pyridin-3-yl]-N-cyclopropylacetamide dihydrochloride

1) A mixture of [5-{[(tert-butoxycarbonyl)amino]methyl}-6-isobutyl-2-methyl-4-(4-methylphenyl)pyridin-3-yl]acetic acid (200 mg, 0.469 mmol), cyclopropylamine (80 mg, 1.41 mmol), 1-hydroxy-1H-benzotriazole (215 mg, 1.41 mmol), 1-ethyl-3-(3-dimethylaminopropyl)carbodiimide hydrochloride (270 mg, 0.65 mmol) and N,N-dimethylformamide (5 mL) was stirred at room temperature for 16 hrs. The reaction mixture was diluted with ethyl acetate and washed with saturated brine. The organic layer was dried over magnesium sulfate and the solvent was evaporated under reduced pressure. The residue was purified by silica gel column chromatography to give tert-butyl {[5-[2-(cyclopropylamino)-2-oxoethyl]-2-isobutyl-6-methyl-4-(4-methylphenyl)pyridin-3-yl]methyl}carbamate (150 mg, yield 69%) as a white powder.

¹H-NMR (CDCl₃) δ:0.33-0.39 (2H, m), 0.97 (6H, d, J=6.6 Hz), 1.38 (9H, s), 1.80 (3H, s), 2.13-2.29 (1H, m), 2.40 (3H, s), 2.54 (3H, s), 2.57-2.64 (1H, m), 2.75 (2H, d, J=7.4 Hz), 3.23 (2H, s), 4.05 (2H, s), 4.20 (1H, brs), 6.94 (2H, d, J=7.9 Hz), 7.23 (2H, d, J=7.9 Hz).

2) 2-[5-(Aminomethyl)-6-isobutyl-2-methyl-4-(4-methylphenyl)pyridin-3-yl]-N-cyclopropylacetamide dihydrochloride (100 mg, yield 89%) was obtained as a white powder from tert-butyl {[5-[2-(cyclopropylamino)-2-oxoethyl]-2-isobutyl-6-methyl-4-(4-methylphenyl)pyridin-3-yl]methyl}carbamate (120 mg, 0.258 mmol) according to a method similar to the method of Example 2-3).

¹H-NMR (DMSO-d₆)δ:0.34 (2H, s), 0.57 (2H, d, J=5.5 Hz), 0.99 (6H, d, J=6.2 Hz), 2.11-2.25 (1H, m), 2.41 (3H, s), 2.53-2.58 (1H, m), 2.81 (2H, s), 3.24 (2H, S), 3.6-3.9 (5H, m), 7.20 (2H, d, J=7.7 Hz), 7.37 (2H, d, J=7.7 Hz), 8.08 (1H, d, J=3.4 Hz), 8.56 (3H, brs).

### Example 312

### {[2-isobutyl-6-methyl-4-(4-methylphenyl)-5-(2-morpholin-4-yl-2-oxoethyl)pyridin-3-yl]methyl}amine dihydrochloride

1) tert-Butyl {[2-isobutyl-6-methyl-4-(4-methylphenyl)-5-(2-morpholin-4-yl-2-oxoethyl)pyridin-3-yl]methyl}carbamate (50 mg, yield 22%) was obtained as a white powder from [5-{[(tert-butoxycarbonyl)amino]methyl}-6-isobutyl-2-methyl-4-(4-methylphenyl)pyridin-3-yl]acetic acid (200 mg, 0.469 mmol) and morpholine (123 mg, 1.41 mmol) according to a method similar to the method of Example 311-1).

¹H-NMR (CDCl₃) δ: 0.97 (6H, d, J=6.6 Hz), 1.37 (9H, s), 2.09-2.27 (1H, m), 2.41 (3H, s), 2.50 (3H, s), 2.73 (2H, d, J=7.4 Hz), 3.17 (2H, d, J=4.1 Hz), 3.30 (2H, s), 3.41 (2H, d, J=4.1 Hz), 3.56 (4H, dd, J=16.5, 4.1 Hz), 4.04 (2H, d, J=4.52 Hz), 4.20 (1H, brs), 6.98 (2H, d, J=7.9 Hz), 7.22 (2H, d, J=7.9 Hz).

2) {[2-Isobutyl-6-methyl-4-(4-methylphenyl)-5-(2-morpholin-4-yl-2-oxoethyl)pyridin-3-yl]methyl}amine dihydrochloride (40 mg, yield 94%) was obtained as a white powder from tert-butyl {[2-isobutyl-6-methyl-4-(4-methylphenyl)-5-(2-morpholin-4-yl-2-oxoethyl)pyridin-3-yl]methyl}carbamate (45 mg, 0.0908 mmol) according to a method similar to the method of Example 2-3).

¹H-NMR (DMSO-d₆)δ:0.99 (6H, d, J=6.4 Hz), 2.09-2.30 (1H, m), 2.41 (3H, s), 2.50 (3H, s), 2.79 (2H, s), 3.09-3.42 (10H, m), 3.82 (2H, d, J=3.8 Hz), 7.16 (2H, d, J=7.7 Hz), 7.39 (2H, d, J=7.7 Hz), 8.52 (3H, brs).

### Example 313

### 2-[5-(aminomethyl)-6-isobutyl-2-methyl-4-(4-methylphenyl)pyridin-3-yl]-N-benzylacetamide dihydrochloride

1) tert-Butyl {[5-[2-(benzylamino)-2-oxoethyl]-2-isobutyl-6-methyl-4-(4-methylphenyl)pyridin-3-yl]methyl}carbamate (150 mg, yield 62%) was obtained as a white powder from [5-{[(tert-butoxycarbonyl)amino]methyl}-6-isobutyl-2-methyl-4-(4-methylphenyl)pyridin-3-yl]acetic acid (200 mg, 0.469 mmol) and benzylamine (151 mg, 1.41 mmol) according to a method similar to the method of Example 311-1).

¹H-NMR (CDCl₃) δ: 0.96 (6H, d, J=6.6 Hz), 1.37 (9H, s), 2.12-2.27 (1H, m), 2.37 (3H, s), 2.56 (3H, s), 2.74 (2H, d, J=7.2 Hz), 3.32 (2H, s), 4.02 (2H, d, J=5.1 Hz), 4.20 (1H, brs), 4.34 (2H, d, J=5.8 Hz), 5.45 (1H, brs), 6.88 (2H, d, J=7.9 Hz), 7.10-7.20 (4H, m), 7.25-7.35 (3H, m).

2) 2-[5-(Aminomethyl)-6-isobutyl-2-methyl-4-(4-methylphenyl)pyridin-3-yl]-N-benzylacetamide dihydrochloride (125 mg, yield 100%) was obtained as a white powder from tert-butyl {[5-[2-(benzylamino)-2-oxoethyl]-2-isobutyl-6-methyl-4-(4-methylphenyl)pyridin-3-yl]methyl}carbamate (130 mg, 0.252 mmol) according to a method similar to the method of Example 2-3).

¹H-NMR (DMSO-d₆)δ:0.99 (6H, d, J=6.4 Hz), 2.07-2.28 (1H, m), 2.40 (3H, s), 2.83 (3H, s), 3.28 (2H, d, J=7.0 Hz), 3.42 (2H s), 3.81 (2H, d, J=3.0 Hz), 4.21 (2H, d, J=5.7 Hz), 7.10-7.44 (9H, m), 8.52 (3H, brs).

### Example 314

### [(2-isobutyl-6-methyl-4-(4-methylphenyl)-5-{[2-(1H-tetrazol-5-yl) phenoxy]methyl}pyridin-3-yl)methyl]amine dihydrochloride

1) tert-Butyl {[5-[(2-cyanophenoxy)methyl]-2-isobutyl-6-methyl-4-(4-methylphenyl)pyridin-3-yl]methyl}carbamate (586 mg, yield 70%) was obtained as a colorless oil from tert-butyl {[5-(hydroxymethyl)-2-isobutyl-6-methyl-4-(4-methylphenyl)pyridin-3-yl]methyl}carbamate (0.67 g, 1.68 mmol) and 2-hydroxybenzonitrile (221 mg, 1.85 mmol) according to a method similar to the method of Example 214-1).

¹H-NMR (CDCl₃) δ:1.00 (6H, d, J=6.6 Hz), 1.39 (9H, s), 2.19-2.28 (1H, m), 2.34 (3H, s), 2.66 (3H, s), 2.79 (2H, d, J=7.2 Hz), 4.09-4.11 (2H, m), 4.26 (1H, brs), 4.73 (2H, s), 6.76 (1H, d, J=8.5 Hz), 6.96-7.01 (2H, m), 7.09 (2H, d, J=8.1 Hz), 7.18 (2H, d, J=7.9 Hz), 7.40-7.46 (1H, m), 7.50-7.56 (1H, m).

2) tert-Butyl [(2-isobutyl-6-methyl-4-(4-methylphenyl)-5-{[2-(1H-tetrazol-5-yl)phenoxy]methyl}pyridin-3-yl)methyl]carbamate (400 mg, yield 63%) was obtained as a white solid from tert-butyl {[5-[(2-cyanophenoxy)methyl]-2-isobutyl-6-methyl-4-(4-methylphenyl)pyridin-3-yl]methyl}carbamate (586 mg, 1.17 mmol) according to a method similar to the method of Example 251-1).

¹H-NMR (CDCl₃) δ:0.99 (6H, d, J=6.6 Hz), 1.39 (9H, s), 2.17-2.28 (1H, m), 2.32 (3H, s), 2.59 (3H, s), 2.82 (2H, d, J=7.4 Hz), 4.09-4.13 (2H, m), 4.31 (1H, brs), 4.92 (2H, s), 6.91-6.95 (3H, m), 7.12 (2H, d, J=7.7 Hz), 7.18 (1H, t, J=7.6 Hz), 7.43-7.49 (1H, m), 8.42 (2H, dd, J=7.9, 1.7 Hz).

3) [(2-Isobutyl-6-methyl-4-(4-methylphenyl)-5-{[2-(1H-tetrazol-5-yl)phenoxy]methyl}pyridin-3-yl)methyl]amine dihydrochloride (327 mg, yield 86%) was obtained as a white solid from tert-butyl [(2-isobutyl-6-methyl-4-(4-methylphenyl)-5-{[2-(1H-tetrazol-5-yl)phenoxy]methyl}pyridin-3-yl)methyl]carbamate (400 mg, 0.737 mmol) according to a method similar to the method of Example 2-3).

¹H-NMR (DMSO-d₆) δ:1.01 (6H, d, J=6.6 Hz), 2.17-2.29 (4H, m), 2.88 (3H, brs), 3.16 (2H, brs), 3.80 (2H, brs), 4.89 (2H, s), 7.03-7.10 (3H, m), 7.13-7.17 (3H, m), 7.46-7.52 (1H, m), 7.87 (1H, d, J=7.7 Hz), 8.41 (3H, brs).

### Example 315

### 5-{[5-(aminomethyl)-6-isobutyl-2-methyl-4-(4-methylphenyl)pyridin-3-yl]methylene}-1,3-thiazolidine-2,4-dione dihydrochloride

1) A mixture of tert-butyl {[5-formyl-2-isobutyl-6-methyl-4-(4-methylphenyl)pyridin-3-yl]methyl}carbamate (600 mg, 1.51 mmol), 1,3-thiazolidine-2,4-dione (177 mg, 1.51 mmol), piperidine (0.015 mL) and ethanol (10 mL) was stirred with heating at 80° C. for 3.5 days. After allowing to cool to room temperature, the solvent was evaporated under reduced pressure. The residue was purified by silica gel column chromatography to give tert-butyl {[5-[(2,4-dioxo-1,3-thiazolidin-5-ylidene)methyl]-2-isobutyl-6-methyl-4-(4-methylphenyl)pyridin-3-yl]methyl}carbamate (400 mg, yield 53%) as a white powder.

¹H-NMR (CDCl₃) δ: 0.98 (6H, d, J=6.6 Hz), 1.39 (9H, s), 2.12-2.31 (1H, m), 2.38 (3H, s), 2.50 (3H, s), 2.78 (2H, d, J=7.4 Hz), 4.12 (2H, d, J=5.1 Hz), 4.20 (1H, brs), 6.96 (2H, d, J=8.1 Hz), 7.19 (2H, d, J=8.1 Hz), 7.51 (1H, s).

2) 5-{[5-(Aminomethyl)-6-isobutyl-2-methyl-4-(4-methylphenyl)pyridin-3-yl]methylene}-1,3-thiazolidine-2,4-dione dihydrochloride (155 mg, yield 100%) was obtained as a white powder from tert-butyl {[5-[(2,4-dioxo-1,3-thiazolidin-5-ylidene)methyl]-2-isobutyl-6-methyl-4-(4-methylphenyl)pyridin-3-yl]methyl}carbamate (157 mg, 0.316 mmol) according to a method similar to the method of Example 2-3).

¹H-NMR (DMSO-d₆)δ:0.99 (6H, d, J=6.4 Hz), 2.14-2.29 (1H, m), 2.37 (3H, s), 2.51 (3H, s), 3.08 (2H, d, J=6.4 Hz), 3.83 (2H, d, J=4.7 Hz), 7.23 (2H, d, J=8.1 Hz), 7.28-7.40 (3H, m), 8.49 (3H, brs).

### Example 316

### 2-{[5-(aminomethyl)-6-isobutyl-2-methyl-4-(4-methylphenyl)pyridin-3-yl]methoxy}-3-methylbenzoic acid dihydrochloride

1) 2-{[5-{[(tert-Butoxycarbonyl)amino]methyl}-6-isobutyl-2-methyl-4-(4-methylphenyl)pyridin-3-yl]methoxy}-3-methylbenzoic acid (280 mg, yield 93%) was obtained as a white powder from methyl 2-{[5-{[(tert-butoxycarbonyl)amino]methyl}-6-isobutyl-2-methyl-4-(4-methylphenyl)pyridin-3-yl]methoxy}-3-methylbenzoate (300 mg, 0.563 mmol) according to a method similar to the method of Example 9-1).

¹H-NMR (CDCl₃) δ:1.07 (6H, d, J=6.4 Hz), 1.38 (9H, s), 1.96 (3H, s), 2.24-2.32 (1H, m), 2.36 (3H, s), 3.14 (3H, s), 3.31 (2H, d, J=6.8 Hz), 4.06 (2H, d, J=4.3 Hz), 4.20 (1H, brs), 4.83 (2H, s), 6.60 (2H, d, J=7.5 Hz), 7.02-7.13 (3H, m), 7.19-7.24 (1H, m), 7.45-7.54 (1H, m).

2) 2-{[5-(Aminomethyl)-6-isobutyl-2-methyl-4-(4-methylphenyl)pyridin-3-yl]methoxy}-3-methylbenzoic acid dihydrochloride (55 mg, yield 100%) was obtained as a white powder from 2-{[5-{[(tert-butoxycarbonyl)amino]methyl}-6-isobutyl-2-methyl-4-(4-methylphenyl)pyridin-3-yl]methoxy}-3-methylbenzoic acid (58.4 mg, 0.110 mmol) according to a method similar to the method of Example 2-3).

¹H-NMR (DMSO-d₆)δ:1.00 (6H, d, J=6.4 Hz), 1.79 (3H, s), 2.14-2.28 (1H, m), 2.36 (3H, s), 2.97 (3H, s), 3.26 (2H, d, J=6.8 Hz), 3.77 (2H, d, J=4.0 Hz), 4.81 (2H, s), 6.93 (2H, d, J=7.9 Hz), 7.09 (1H, t, J=7.5 Hz), 7.19 (2H, d, J=7.9 Hz), 7.29 (1H, d, J=6.6 Hz), 7.38-7.46 (1H, m), 8.57 (3H, brs).

### Example 317

### 2-{[5-(aminomethyl)-6-isobutyl-2-methyl-4-(4-methylphenyl)pyridin-3-yl]methoxy}-5-chlorobenzamide dihydrochloride

1) 2-{[5-{[(tert-Butoxycarbonyl)amino]methyl}-6-isobutyl-2-methyl-4-(4-methylphenyl)pyridin-3-yl]methoxy}-5-chlorobenzoic acid (0.54 g, yield 97%) was obtained as a white powder from methyl 2-{[5-{[(tert-butoxycarbonyl)amino]methyl}-6-isobutyl-2-methyl-4-(4-methylphenyl)pyridin-3-yl]methoxy}-5-chlorobenzoate (0.57 g, 1.0 mmol) according to a method similar to the method of Example 43-1).

¹H-NMR (CDCl₃)δ: 1.04 (6H, d, J=6.6 Hz), 1.37 (9H, s), 2.20-2.35 (1H, m), 2.40 (3H, s), 3.00 (3H, s), 3.21 (2H, d, J=5.2 Hz), 4.17 (2H, d, J=5.8 Hz), 4.50-4.65 (1H, m), 4.88 (2H, s), 6.62 (1H, d, J=8.9 Hz), 7.05 (2H, d, J=7.8 Hz), 7.25 (2H, d, J=7.8 Hz), 7.33 (1H, dd, J=2.6, 8.9 Hz), 7.90 (1H, d, J=8.9 Hz).

2) tert-Butyl {[5-{[2-(aminocarbonyl)-4-chlorophenoxy]methyl}-2-isobutyl-6-methyl-4-(4-methylphenyl)pyridin-3-yl]methyl}carbamate (0.20 g, yield 71%) was obtained as a white powder from 2-{[5-{[(tert-butoxycarbonyl)amino]methyl}-6-isobutyl-2-methyl-4-(4-methylphenyl)pyridin-3-yl]methoxy}-5-chlorobenzoic acid (0.28 g, 0.51 mmol) according to a method similar to the method of Example 3-1).

¹H-NMR (CDCl₃)δ: 0.99 (6H, d, J=6.6 Hz), 1.39 (9H, s), 2.15-2.35 (1H, m,) 2.36 (3H, s), 2.63 (3H, s), 2.80 (2H, d, J=7.4 Hz), 4.10 (2H, d, J=5.1 Hz), 4.15-4.30 (1H, m), 4.77 (2H, s), 5.65 (1H, brs), 6.69 (1H, d, J=8.9 Hz), 6.99 (2H, d, J=7.9 Hz), 7.18 (2H, d, J=7.9 Hz), 7.31 (1H, dd, J=2.8, 8.9 Hz), 7.48 (1H, brs), 8.18 (1H, d, J=2.8 Hz).

3) 2-{[5-(Aminomethyl)-6-isobutyl-2-methyl-4-(4-methylphenyl)pyridin-3-yl]methoxy}-5-chlorobenzamide dihydrochloride (0.16 g, yield 99%) was obtained as a white powder from tert-butyl {[5-{[2-(aminocarbonyl)-4-chlorophenoxy]methyl}-2-isobutyl-6-methyl-4-(4-methylphenyl)pyridin-3-yl]methyl}carbamate (0.17 g, 0.31 mmol) according to a method similar to the method of Example 2-3).

¹H-NMR (DMSO-d₆)δ:0.99 (6H, d, J=6.6 Hz), 2.15-2.35 (1H, m), 2.36 (3H, s), 2.84 (3H, brs), 3.08 (2H, brs), 3.82 (2H, d, J=2.6 Hz), 4.79 (2H, s), 6.83 (1H, d, J=9.0 Hz), 7.25 (2H, d, J=7.9 Hz), 7.31 (2H, d, J=7.9 Hz), 7.41 (1H, dd, J=2.7, 9.0 Hz), 7.52 (2H, brs), 7.55 (1H, d, J=2.7 Hz), 8.36 (3H, brs).

### Example 318

### 2-{[5-(aminomethyl)-6-isobutyl-2-methyl-4-(4-methylphenyl)pyridin-3-yl]methoxy}-5-chlorobenzoic acid dihydrochloride

2-{[5-(Aminomethyl)-6-isobutyl-2-methyl-4-(4-methylphenyl)pyridin-3-yl]methoxy}-5-chlorobenzoic acid dihydrochloride (0.16 g, yield 85%) was obtained as a white powder from 2-{[5-{[(tert-butoxycarbonyl)amino]methyl}-6-isobutyl-2-methyl-4-(4-methylphenyl)pyridin-3-yl]methoxy}-5-chlorobenzoic acid (0.20 g, 0.36 mmol) according to a method similar to the method of Example 276-3).

¹H-NMR (DMSO-d₆)δ:0.99 (6H, d, J=6.6 Hz), 2.15-2.30 (1H, m), 2.36 (3H, s), 2.83 (3H, brs), 3.05 (2H, brs), 3.75-3.90 (2H, m), 4.77 (2H, brs), 6.92 (1H, d, J=8.9 Hz), 7.24 (2H, d, J=7.8 Hz), 7.31 (2H, d, J=7.8 Hz), 7.47 (1H, dd, J=2.8, 8.9 Hz), 7.61 (1H, d, J=2.8 Hz), 8.30 (3H, brs).

### Example 319

### 4′-[({[5-(aminomethyl)-6-isobutyl-2-methyl-4-(4-methylphenyl)pyridin-3-yl]carbonyl}oxy)methyl]biphenyl-4-carboxylic acid dihydrochloride

1) 4-Bromobenzyl 5-{[(tert-butoxycarbonyl)amino]methyl}-6-isobutyl-2-methyl-4-(4-methylphenyl)nicotinate (1.92 g, yield 75%) was obtained as a colorless oil from 5-{[(tert-butoxycarbonyl)amino]methyl}-6-isobutyl-2-methyl-4-(4-methylphenyl)nicotinic acid (1.82 g, 4.41 mmol) and 4-bromobenzyl bromide (1.10 g, 4.41 mmol) according to a method similar to the method of Example 169-1).

¹H-NMR (CDCl₃) δ:0.96 (6H, d, J=6.6 Hz), 1.38 (9H, s), 2.15-2.26 (1H, m), 2.38 (3H, s), 2.53 (3H, s), 2.77 (2H, d, J=7.2 Hz), 4.11 (2H, d, J=4.9 Hz), 4.19 (1H, brs), 4.89 (2H, s), 6.91 (2H, d, J=8.5 Hz), 6.99 (2H, d, J=8.1 Hz), 7.09 (2H, d, J=7.7 Hz), 7.39 (2H, d, J=8.5 Hz).

2) A solution of 4-bromobenzyl 5-{[(tert-butoxycarbonyl)amino]methyl}-6-isobutyl-2-methyl-4-(4-methylphenyl)nicotinate (1.09 g, 1.87 mmol), [4-(methoxycarbonyl)phenyl]boronic acid (675 mg, 3.75 mmol), potassium carbonate (388 mg, 2.81 mmol) and tetrakis(triphenylphosphine)palladium(0) (216 mg, 0.187 mmol) in dioxane (15 mL) and water (2.5 mL) was stirred under an argon atmosphere for 12 hrs. The reaction mixture was diluted with ethyl acetate, washed with saturated brine and dried over anhydrous magnesium sulfate. The solvent was evaporated under reduced pressure and the obtained residue was purified by silica gel column chromatography to give [4′-(methoxycarbonyl)biphenyl-4-yl]methyl 5-{[(tert-butoxycarbonyl)amino]methyl}-6-isobutyl-2-methyl-4-(4-methylphenyl)nicotinate (570 mg, yield 48%) as a colorless oil.

¹H-NMR (CDCl₃) δ:0.96 (6H, d, J=6.6 Hz), 1.38 (9H, s), 2.17-2.26 (1H, m), 2.29 (3H, s), 2.55 (3H, s), 2.78 (2H, d, J=7.4 Hz), 3.91 (3H, s), 4.16 (2H, d, J=4.5 Hz), 4.60 (1H, brs), 4.98 (2H, s), 7.07 (2H, d, J=8.1 Hz), 7.12-7.16 (4H, m), 7.53 (2H, d, J=8.3 Hz), 7.64 (2H , J=8.7 Hz), 8.10 (2H, d, J=8.5 Hz).

3) 4′-[({[5-{[(tert-Butoxycarbonyl)amino]methyl}-6-isobutyl-2-methyl-4-(4-methylphenyl)pyridin-3-yl]carbonyl}oxy)methyl]biphenyl-4-carboxylic acid (380 mg, yield 68%) was obtained as a white solid from [4′-(methoxycarbonyl)biphenyl-4-yl]methyl 5-{[(tert-butoxycarbonyl)amino]methyl}-6-isobutyl-2-methyl-4-(4-methylphenyl)nicotinate (570 mg, 0.895 mmol) according to a method similar to the method of Example 9-1).

¹H-NMR (CDCl₃) δ:0.96 (6H, d, J=6.6 Hz), 1.39 (9H, s), 2.15-2.26 (1H, m), 2.34 (3H, s), 2.56 (3H, s), 2.79 (2H, d, J=7.4 Hz), 4.11-4.16 (2H, m), 4.23 (1H, brs), 4.99 (2H, s), 7.05 (2H, d, J=7.9 Hz), 7.13-7.18 (4H, m), 7.55 (2H, d, J=8.3 Hz), 7.68 (2H, d, J=8.5 Hz), 8.18 (2H, d, J=8.3 Hz).

4) 4′-[({[5-(Aminomethyl)-6-isobutyl-2-methyl-4-(4-methylphenyl)pyridin-3-yl]carbonyl}oxy)methyl]biphenyl-4-carboxylic acid dihydrochloride (255 mg, yield 70%) was obtained as a white solid from 4′-[({[5-{[(tert-butoxycarbonyl)amino]methyl}-6-isobutyl-2-methyl-4-(4-methylphenyl)pyridin-3-yl]carbonyl}oxy)methyl]biphenyl-4-carboxylic acid (380 mg, 0.610 mmol) according to a method similar to the method of Example 2-3).

¹H-NMR (DMSO-d₆) δ:0.96 (6H, d, J=6.6 Hz), 2.15-2.26 (1H, m), 2.33 (3H, s), 2.57 (3H, brs), 2.92 (2H, brs), 3.82 (2H, d, J=4.3 Hz), 5.04 (2H, s), 7.18 (4H, d, J=8.3 Hz), 7.24 (2H, d, J=8.1 Hz), 7.68 (2H, d, J=8.3 Hz), 7.82 (2H, d, J=8.5 Hz), 8.04 (2H, d, J=8.5 Hz), 8.34 (3H, brs).

### Example 320

### pyridin-4-ylmethyl 5-(aminomethyl)-6-isobutyl-2-methyl-4-(4-methylphenyl)nicotinate trihydrochloride

1) Pyridin-4-ylmethyl 5-{[(tert-butoxycarbonyl)amino]methyl}-6-isobutyl-2-methyl-4-(4-methylphenyl)nicotinate (322 mg, yield 53%) was obtained as a colorless oil from 5-{[(tert-butoxycarbonyl)amino]methyl}-6-isobutyl-2-methyl-4-(4-methylphenyl)nicotinic acid (0.50 g, 1.21 mmol), 4-(chloromethyl)pyridine hydrochloride (0.20 g, 1.21 mmol) and potassium carbonate (0.42 g, 3.0 mmol) according to a method similar to the method of Example 169-1).

¹H-NMR (CDCl₃)δ:0.97 (6H, d, J=6.6 Hz), 1.39 (9H, s), 2.17-2.27 (1H, m), 2.36 (3H, s), 2.56 (3H, s), 2.78 (2H, d, J=7.4 Hz), 4.14 (2H, d, J=4.9 Hz), 4.42 (1H, brs), 4.94 (2H, s), 6.89 (2H, d, J=5.8 Hz), 7.04 (2H, d, J=8.1 Hz), 7.12 (2H, d, J=7.9 Hz), 8.48 (2H, d, J=5.3 Hz).

2) Pyridin-4-ylmethyl 5-(aminomethyl)-6-isobutyl-2-methyl-4-(4-methylphenyl)nicotinate trihydrochloride (260 mg, yield 79%) was obtained as a white solid from pyridin-4-ylmethyl 5-{[(tert-butoxycarbonyl)amino]methyl}-6-isobutyl-2-methyl-4-(4-methylphenyl)nicotinate (322 mg, 0.639 mmol) according to a method similar to the method of Example 2-3).

¹H-NMR (DMSO-d₆) δ:0.97 (6H, d, J=6.6 Hz), 2.19-2.27 (1H, m), 2.33 (3H, s), 2.57 (3H, brs), 2.89 (2H, brs), 3.81 (2H, d, J=5.5 Hz), 5.29 (2H, s), 7.17-7.24 (4H, m), 7.60 (2H, brs), 8.35 (3H, brs), 8.83-8.84 (2H, brs).

### Example 321

### pyridin-3-ylmethyl 5-(aminomethyl)-6-isobutyl-2-methyl-4-(4-methylphenyl)nicotinate trihydrochloride

1) Pyridin-3-ylmethyl 5-{[(tert-butoxycarbonyl)amino]methyl}-6-isobutyl-2-methyl-4-(4-methylphenyl)nicotinate (454 mg, yield 74%) was obtained as a colorless oil from 5-{[(tert-butoxycarbonyl)amino]methyl}-6-isobutyl-2-methyl-4-(4-methylphenyl)nicotinic acid (0.50 g, 1.21 mmol), 3-(bromomethyl)pyridine hydrobromide (0.46 g, 1.81 mmol) and potassium carbonate (0.50 g, 3.6 mmol) according to a method similar to the method of Example 169-1).

¹H-NMR (CDCl₃)δ:0.96 (6H, d, J=6.6 Hz), 1.38 (9H, s), 2.15-2.24 (1H, m), 2.36 (3H, s), 2.54 (3H, s), 2.77 (2H, d, J=7.4 Hz), 4.12 (2H, d, J=4.1 Hz), 4.20 (1H, brs), 4.94 (2H, s), 6.99 (2H, d, J=8.1 Hz), 7.09 (2H, d, J=7.9 Hz), 7.17-7.21 (1H, m), 7.32-7.37 (1H, m), 8.34 (1H, d, J=1.7 Hz), 8.55 (1H, dd, J=4.8, 1.6 Hz).

2) Pyridin-3-ylmethyl 5-(aminomethyl)-6-isobutyl-2-methyl-4-(4-methylphenyl)nicotinate trihydrochloride (183 mg, yield 39%) was obtained as a white solid from pyridin-3-ylmethyl 5-{[(tert-butoxycarbonyl)amino]methyl}-6-isobutyl-2-methyl-4-(4-methylphenyl)nicotinate (454 mg, 0.903 mmol) according to a method similar to the method of Example 2-3).

¹H-NMR (DMSO-d₆) δ:0.96 (6H, d, J=6.8 Hz), 2.17-2.26 (1H, m), 2.31 (3H, s), 2.59 (3H, s), 2.93 (2H, d, J=6.0 Hz), 3.78 (2H, d, J=5.5 Hz), 5.22 (2H, s), 7.12 (4H, s), 7.95 (1H, t, J=6.7 Hz), 8.14 (1H, d, J=7.9 Hz), 8.41 (3H, brs), 8.67 (1H, s), 8.90 (1H, d, J=5.5 Hz).

### Example 322

### methyl 2-{[5-(aminomethyl)-6-isobutyl-2-methyl-4-(4-methylphenyl)pyridin-3-yl]methoxy}-3-methoxybenzoate dihydrochloride

1) Methyl 2-{[5-{[(tert-butoxycarbonyl)amino]methyl}-6-isobutyl-2-methyl-4-(4-methylphenyl)pyridin-3-yl]methoxy}-3-methoxybenzoate (0.62 g, yield 55%) was obtained as a white powder from tert-butyl {[5-(hydroxymethyl)-2-isobutyl-6-methyl-4-(4-methylphenyl)pyridin-3-yl]methyl}carbamate (0.80 g, 2.0 mmol) and methyl 3-methoxysalicylate (0.55 g, 3.0 mmol) according to a method similar to the method of Example 106-1).

¹H-NMR (CDCl₃)δ: 0.98 (6H, d, J=6.6 Hz), 1.38 (9H, s), 2.15-2.30 (1H, m), 2.34 (3H, s), 2.73 (3H, s), 2.75 (2H, d, J=7.4 Hz), 3.54 (3H, s), 3.64 (3H, s), 3.97 (2H, d, J=5.1 Hz), 4.20-4.30 (1H, m), 4.86 (2H, s), 6.60 (2H, d, J=8.1 Hz), 6.85 (1H, dd, J=1.5, 8.1 Hz), 7.01 (2H, d, J=8.1 Hz), 7.06 (1H, d, J=8.1 Hz), 7.14 (1H, dd, J=1.5, 8.1 Hz).

2) Methyl 2-{[5-(aminomethyl)-6-isobutyl-2-methyl-4-(4-methylphenyl)pyridin-3-yl]methoxy}-3-methoxybenzoate dihydrochloride (0.12 g, yield 66%) was obtained as a white powder from methyl 2-{[5-{[(tert-butoxycarbonyl)amino]methyl}-6-isobutyl-2-methyl-4-(4-methylphenyl)pyridin-3-yl]methoxy}-3-methoxybenzoate (0.19 g, 0.34 mmol) according to a method similar to the method of Example 274-2).

¹H-NMR (DMSO-d₆)δ: 0.99 (6H, d, J=6.6 Hz), 2.10-2.30 (1H, m), 2.37 (3H, s), 2.94 (3H, brs), 3.00-3.20 (2H, m), 3.51 (3H, s), 3.63 (3H, s), 3.72 (2H, brs), 4.88 (2H, brs), 6.77 (2H, d, J=7.9 Hz), 7.00-7.22 (3H, m), 7.17 (2H, d, J=7.9 Hz), 8.27 (3H, brs).

### Example 323

### methyl 2-({[5-(aminomethyl)-2-methyl-4-(4-methylphenyl)-6-neopentylpyridin-3-yl]methyl}thio)benzoate dihydrochloride

1) Methyl 2-({[5-{[(tert-butoxycarbonyl)amino]methyl}-2-methyl-4-(4-methylphenyl)-6-neopentylpyridin-3-yl]methyl}thio)benzoate (1.46 g, yield 63%) was obtained as a powder from [5-{[(tert-butoxycarbonyl)amino]methyl}-2-methyl-4-(4-methylphenyl)-6-neopentylpyridin-3-yl]methyl methanesulfonate (2.0 g, 4.7 mmol) and methyl thiosalicylate (757 mg, 45 mmol) according to a method similar to the method of Example 33-1).

¹H-NMR (CDCl₃) δ:1.02 (9H, s), 1.37 (9H, s), 2.34 (3H, s), 2.65 (3H, s), 2.83 (2H, s), 3.89 (3H, s), 4.07 (2H, d, J=4.9 Hz), 4.17 (1H, brs), 7.04-7.18 (6H, m), 7.32-7.38 (1H, m), 7.91-7.95 (1H, m).

2) Methyl 2-({[5-(aminomethyl)-2-methyl-4-(4-methylphenyl)-6-neopentylpyridin-3-yl]methyl}thio)benzoate dihydrochloride (254 mg, yield 89%) was obtained as a powder from methyl 2-({[5-{[(tert-butoxycarbonyl)amino]methyl}-2-methyl-4-(4-methylphenyl)-6-neopentylpyridin-3-yl]methyl}thio)benzoate (300 mg, 0.533 mmol) according to a method similar to the method of Example 2-3).

¹H-NMR (DMSO-d₆) δ:1.03 (9H, s), 2.34 (3H, s), 2.83 (3H, s), 3.18 (2H, brs), 3.80 (3H, s), 3.88 (2H, s), 4.00 (2H, s), 7.23-7.32 (6H, m), 7.47-7.52 (1H, m), 7.85-7.88 (1H, m), 8.21 (3H, brs).

### Example 324

### 2-({[5-(aminomethyl)-2-methyl-4-(4-methylphenyl)-6-neopentylpyridin-3-yl]methyl}thio)benzoic acid dihydrochloride

1) 4-({[5-{[(tert-Butoxycarbonyl)amino]methyl}-2-methyl-4-(4-methylphenyl)-6-neopentylpyridin-3-yl]methyl}thio)benzoic acid (897 mg, yield 92%) was obtained as a white solid from methyl 4-({[5-{[(tert-butoxycarbonyl)amino]methyl}-2-methyl-4-(4-methylphenyl)-6-neopentylpyridin-3-yl]methyl}thio)benzoate (1.0 g, 1.78 mmol) according to a method similar to the method of Example 9-1).

¹H-NMR (CDCl₃) δ:1.12 (9H, s), 1.38 (9H, s), 2.38 (3H, s), 3.09 (3H, s), 3.47 (2H, s), 3.79 (2H, s), 4.14 (2H, d, J=4.3 Hz), 4.52 (1H, brs), 6.85-6.92 (2H, m), 7.08-7.13 (1H, m), 7.19-7.21 (2H, m), 7.29-7.33 (1H, m), 7.37-7.41 (1H, m), 7.94-7.97 (1H, m)

2) 2-({[5-(Aminomethyl)-2-methyl-4-(4-methylphenyl)-6-neopentylpyridin-3-yl]methyl}thio)benzoic acid dihydrochloride (158 mg, yield 83%) was obtained as a white powder from 4-({[5-{[(tert-butoxycarbonyl)amino]methyl}-2-methyl-4-(4-methylphenyl)-6-neopentylpyridin-3-yl]methyl}thio)benzoic acid (200 mg, 0.364 mmol) according to a method similar to the method of Example 2-3).

¹H-NMR (DMSO-d₆) δ:1.03 (9H, s), 2.34 (3H, s), 2.81 (3H, s), 3.15 (2H, brs), 3.80 (2H, s), 3.85 (2H, s), 7.19-7.33 (6H, m), 7.44-7.49 (1H, m), 7.86-7.89 (1H, m), 8.17 (3H, brs).

### Example 325

### 2-({[5-(aminomethyl)-2-methyl-4-(4-methylphenyl)-6-neopentylpyridin-3-yl]methyl}thio)benzamide dihydrochloride

1) 4-({[5-{[(tert-Butoxycarbonyl)amino]methyl}-2-methyl-4-(4-methylphenyl)-6-neopentylpyridin-3-yl]methyl}thio)benzamide (349 mg, yield 70%) was obtained as a white solid from 4-({[5-{[(tert-butoxycarbonyl)amino]methyl}-2-methyl-4-(4-methylphenyl)-6-neopentylpyridin-3-yl]methyl}thio)benzoic acid (500 mg, 0.911 mmol) according to a method similar to the method of Example 3-1).

¹H-NMR (CDCl₃) δ:1.02 (9H, s), 1.37 (9H, s), 2.39 (3H, s), 2.63 (3H, s), 2.83 (2H, s), 3.81 (2H, s), 4.04 (2H, d, J=5.1 Hz), 4.24 (1H, brs), 5.45 (1H, brs), 6.68 (1H, brs), 6.96-6.99 (2H, m), 7.18-7.22 (3H, m), 7.28-7.32 (2H, m), 7.75-7.78 (1H, m). 2) 2-({[5-(Aminomethyl)-2-methyl-4-(4-methylphenyl)-6-neopentylpyridin-3-yl]methyl}thio)benzamide dihydrochloride (160 mg, yield 84%) was obtained as a white powder from 4-({[5-{[(tert-butoxycarbonyl)amino]methyl}-2-methyl-4-(4-methylphenyl)-6-neopentylpyridin-3-yl]methyl}thio)benzamide (200 mg, 0.365 mmol) according to a method similar to the method of Example 2-3).

¹H-NMR (DMSO-d₆) δ:1.03 (9H, s), 2.37 (3H, s), 2.76 (3H, s), 3.17 (2H, brs), 3.75-3.85 (4H, m), 7.14-7.35 (7H, m), 7.40 (1H, s), 7.50-7.48 (1H, m), 7.81 (1H, s), 8.20 (3H, brs).

### Example 326

### 2-{[5-(aminomethyl)-6-isobutyl-2-methyl-4-(4-methylphenyl)pyridin-3-yl]methoxy}-3-methylbenzamide dihydrochloride

1) tert-Butyl {[5-{[2-(aminocarbonyl)-6-methylphenoxy]methyl}-2-isobutyl-6-methyl-4-(4-methylphenyl)pyridin-3-yl]methyl}carbamate (190 mg, yield 95%) was obtained as a white powder from 2-{[5-{[(tert-butoxycarbonyl)amino]methyl}-6-isobutyl-2-methyl-4-(4-methylphenyl)pyridin-3-yl]methoxy}-3-methylbenzoic acid (200 mg, 0.375 mmol) according to a method similar to the method of Example 3-1).

¹H-NMR (CDCl₃) δ:1.05 (6H, d, J=6.2 Hz), 1.40 (9H, s), 1.93 (3H, s), 2.21-2.32 (1H, m), 2.36 (3H, s), 3.01 (3H, s), 3.16 (2H, d, J=6.8 Hz), 4.04 (2H, s), 4.20 (1H, brs), 4.81 (2H, s),5.80 (1H, brs), 6.40 (1H, brs), 6.65 (2H, s), 7.02-7.23 (4H, m), 7.56 (1H, s).

2) 2-{[5-(Aminomethyl)-6-isobutyl-2-methyl-4-(4-methylphenyl)pyridin-3-yl]methoxy}-3-methylbenzamide dihydrochloride (100 mg, yield 70%) was obtained as a white powder from tert-butyl {[5-{[2-(aminocarbonyl)-6-methylphenoxy]methyl}-2-isobutyl-6-methyl-4-(4-methylphenyl)pyridin-3-yl]methyl}carbamate (150 mg, 0.282 mmol) according to a method similar to the method of Example 2-3).

¹H-NMR (DMSO-d₆) δ:1.00 (6H, d, J=6.4 Hz), 1.76 (3H, s), 2.13-2.29 (1H, m), 2.37 (3H, s), 2.96 (3H, s), 3.21 (2H, d, J=6.6 Hz), 3.76 (2H, d, J=4.9 Hz), 4.78 (2H, s), 7.01 (2H, d, J=7.9 Hz), 7.04-7.08 (1H, m), 7.15-7.26 (4H, m), 7.34 (1H, brs), 7.53 (1H, brs), 8.52 (3H, brs).

### Example 327

### 2-[5-(aminomethyl)-6-isobutyl-2-methyl-4-(4-methylphenyl)pyridin-3-yl]-N-phenylacetamide dihydrochloride

1) tert-Butyl {[5-(2-anilino-2-oxoethyl)-2-isobutyl-6-methyl-4-(4-methylphenyl)pyridin-3-yl]methyl}carbamate (220 mg, yield 94%) was obtained as a white powder from [5-{[(tert-butoxycarbonyl)amino]methyl}-6-isobutyl-2-methyl-4-(4-methylphenyl)pyridin-3-yl]acetic acid (200 mg, 0.469 mmol) and aniline (150 mg, 1.41 mmol) according to a method similar to the method of Example 311-1).

¹H-NMR (CDCl₃) δ:0.98 (6H, d, J=6.6 Hz), 1.38 (9H, s), 2.15-2.29 (1H, m), 2.40 (3H, s), 2.63 (3H, s), 2.77 (2H, d, J=7.2 Hz), 3.66 (3H, s), 4.06 (2H, d, J=4.9Hz), 4.20 (1H, brs), 7.02 (2H, d, J=7.9 Hz), 7.06-7.14 (1H, m), 7.24 (2H, d, J=7.9 Hz), 7.27-7.39 (4H, m).

2) 2-[5-(Aminomethyl)-6-isobutyl-2-methyl-4-(4-methylphenyl)pyridin-3-yl]-N-phenylacetamide dihydrochloride (200 mg, yield 100%) was obtained as a white powder from tert-butyl {[5-(2-anilino-2-oxoethyl)-2-isobutyl-6-methyl-4-(4-methylphenyl)pyridin-3-yl]methyl}carbamate (210 mg, 0.419 mmol) according to a method similar to the method of Example 2-3).

¹H-NMR (DMSO-d₆)δ:1.00 (6H, d, J=5.5 Hz), 2.13-2.28 (1H, m), 2.38 (3H, s), 2.85 (3H, s), 3.25 (2H, s), 3.62 (2H, s), 3.83 (2H, s), 7.04 (1H, t, J=6.7 Hz), 7.15-7.42 (6H, m), 7.50 (2H, d, J=7.4 Hz), 8.53 (3H, brs), 10.20 (1H, s).

### Example 328

### N-[5-(aminomethyl)-6-isobutyl-2-methyl-4-(4-methylphenyl)pyridin-3-yl]cyclohexanecarboxamide dihydrochloride

N-[5-(Aminomethyl)-6-isobutyl-2-methyl-4-(4-methylphenyl)pyridin-3-yl]cyclohexanecarboxamide dihydrochloride (230 mg, yield 98%) was obtained as a white powder from tert-butyl {[5-amino-2-isobutyl-6-methyl-4-(4-methylphenyl)pyridin-3-yl]methyl}carbamate (192 mg, 0.5 mmol) and cyclohexanecarbonyl chloride (100 μL, 0.75 mmol) according to a method similar to the method of Example 223.

¹H-NMR (DMSO-d₆) δ: 0.98 (6H, d, J=6.6 Hz), 1.00-1.25 (6H, m), 1.41 (2H, brs), 1.59 (2H, brs), 2.08-2.22 (2H, m), 2.37 (3H, s), 2.53 (3H, s), 3.03 (2H, brs), 3.81 (2H, s), 7.14 (2H, d, J=7.8 Hz), 7.30 (2H, d, J=7.8 Hz), 8.33 (3H, brs), 9.37 (1H, brs).

### Example 329

### N-[5-(aminomethyl)-6-isobutyl-2-methyl-4-(4-methylphenyl)pyridin-3-yl]piperidine-1-carboxamide dihydrochloride

1) tert-Butyl ({2-isobutyl-6-methyl-4-(4-methylphenyl)-5-[(piperidin-1-ylcarbonyl)amino]pyridin-3-yl}methyl)carbamate was obtained as an oil from 5-{[(tert-butoxycarbonyl)amino]methyl}-6-isobutyl-2-methyl-4-(4-methylphenyl)nicotinic acid (412 mg, 1.0 mmol) and piperidine (150 μL, 1.5 mmol) according to a method similar to the method of Example 95-1).

EIMS(M+1):495

2) N-[5-(Aminomethyl)-6-isobutyl-2-methyl-4-(4-methylphenyl)pyridin-3-yl]piperidine-1-carboxamide dihydrochloride (218 mg, yield 47%) was obtained as a white powder from the oil obtained in aforementioned 1), according to a method similar to the method of Example 2-3).

¹H-NMR (DMSO-d₆)δ:0.98 (6H, d, J=6.3 Hz), 1.07-1.19 (4H, m), 1.44 (2H, brs), 2.12-2.27 (1H, m), 2.37 (3H, s), 2.60 (3H, s), 3.05 (2H, brs), 3.15 (4H, brs), 3.83 (2H, s), 7.19 (2H, d, J=7.8 Hz), 7.31 (2H, d, J=7.8 Hz), 7.96 (1H, brs), 8.27 (3H, brs).

### Example 330

### N-[5-(aminomethyl)-6-isobutyl-2-methyl-4-(4-methylphenyl)pyridin-3-yl]tetrahydro-2H-pyran-4-carboxamide dihydrochloride

N-[5-(Aminomethyl)-6-isobutyl-2-methyl-4-(4-methylphenyl)pyridin-3-yl]tetrahydro-2H-pyran-4-carboxamide dihydrochloride (232 mg, yield 98%) was obtained as a white powder from tert-butyl {[5-amino-2-isobutyl-6-methyl-4-(4-methylphenyl)pyridin-3-yl]methyl}carbamate (192 mg, 0.5 mmol) and tetrahydro-2H-pyran-4-carbonyl chloride (111 mg, 0.75 mmol) according to a method similar to the method of Example 223.

¹H-NMR (DMSO-d₆) δ: 0.98 (6H, d, J=6.6 Hz), 1.00-1.25 (6H, m), 1.41 (2H, brs), 1.59 (2H, brs), 2.08-2.22 (2H, m), 2.37 (3H, s), 2.53 (3H, s), 3.03 (2H, brs), 3.81 (2H, s), 7.14 (2H, d, J=7.5 Hz), 7.30 (2H, d, J=7.8 Hz), 8.27 (3H, brs), 9.43 (1H, brs).

### Example 331

### N-[5-(aminomethyl)-6-isobutyl-2-methyl-4-(4-methylphenyl)pyridin-3-yl]morpholine-4-carboxamide dihydrochloride

1) tert-Butyl ({2-isobutyl-6-methyl-4-(4-methylphenyl)-5-[(morpholin-4-ylcarbonyl)amino]pyridin-3-yl}methyl)carbamate was obtained as an oil from 5-{[(tert-butoxycarbonyl)amino]methyl}-6-isobutyl-2-methyl-4-(4-methylphenyl)nicotinic acid (412 mg, 1.0 mmol) and morpholine (130 μL, 1.5 mmol) according to a method similar to the method of Example 95-1).

EIMS(M+1):497

2) N-[5-(Aminomethyl)-6-isobutyl-2-methyl-4-(4-methylphenyl)pyridin-3-yl]morpholine-4-carboxamide dihydrochloride (278 mg, yield 59%) was obtained as a white powder from the oil obtained in the aforementioned 1), according to a method similar to the method of Example 2-3).

¹H-NMR (DMSO-d₆)δ:0.99 (6H, d, J=6.3 Hz), 2.10-2.27 (1H, m), 2.39 (3H, s), 2.70 (3H, s), 3.14 (6H, brs), 3.19 (4H, brs), 3.86 (2H, brs), 7.21 (2H, d, J=7.8 Hz), 7.34 (2H, d, J=7.8 Hz), 8.44 (4H, brs).

### Example 332

### N-[5-(aminomethyl)-6-isobutyl-2-methyl-4-(4-methylphenyl)pyridin-3-yl]piperidine-4-carboxamide trihydrochloride

N-[5-(Aminomethyl)-6-isobutyl-2-methyl-4-(4-methylphenyl)pyridin-3-yl]piperidine-4-carboxamide trihydrochloride (246 mg, yield 98%) was obtained as a white powder from tert-butyl {[5-amino-2-isobutyl-6-methyl-4-(4-methylphenyl)pyridin-3-yl]methyl}carbamate (192 mg, 0.5 mmol) and benzyl 4-(chlorocarbonyl)piperidine-1-carboxylate (210 mg, 0.75 mmol) according to a method similar to the method of Example 223.

¹H-NMR (DMSO-d₆) δ: 0.98 (6H, d, J=6.6 Hz), 1.44 (4H, brs), 2.15-2.26 (1H, m), 2.38 (3H, s), 2.38-2.57 (1H, m), 2.57 (3H, s), 2.76 (2H, brs), 3.07 (4H, brs), 3.81 (2H, brs), 7.17 (2H, d, J=8.1 Hz), 7.30 (2H, d, J=8.1 Hz), 8.41 (3H, brs), 8.80 (1H, brs), 9.09 (1H, brs), 9.84 (1H, brs).

### Example 333

### N-[5-(aminomethyl)-6-isobutyl-2-methyl-4-(4-methylphenyl)pyridin-3-yl]piperazine-1-carboxamide trihydrochloride

1) tert-Butyl 4-({[5-{[(tert-butoxycarbonyl)amino]methyl}-6-isobutyl-2-methyl-4-(4-methylphenyl)pyridin-3-yl]amino}carbonyl)piperazine-1-carboxylate was obtained as an oil from 5-{[(tert-butoxycarbonyl)amino]methyl}-6-isobutyl-2-methyl-4-(4-methylphenyl)nicotinic acid (412 mg, 1.0 mmol) and tert-butyl piperazine-l-carboxylate (140 mg, 1.5 mmol) according to a method similar to the method of Example 95-1).

EIMS(M+1):596

2) N-[5-(Aminomethyl)-6-isobutyl-2-methyl-4-(4-methylphenyl)pyridin-3-yl]piperazine-1-carboxamide trihydrochloride (250 mg, yield 97%) was obtained as a white powder from the oil obtained in the aforementioned 1), according to a method similar to the method of Example 2-3).

¹H-NMR (DMSO-d₆)δ:0.98 (6H, d, J=6.3 Hz), 2.15-2.26 (1H, m), 2.42 (3H, s), 2.62 (2H, s), 2.72 (3H, s), 3.05 (2H, brs), 3.42 (4H, brs), 3.82 (2H, brs), 7.19 (2H, d, J=7.5 Hz), 7.31 (2H, d, J=7.5 Hz), 8.37 (3H, brs), 8.60 (1H, brs), 9.41 (2H, brs).

### Example 334

### (5-{[5-(aminomethyl)-6-isobutyl-2-methyl-4-(4-methylphenyl)pyridin-3-yl]methylene}-4-oxo-2-thioxo-1,3-thiazolidin-3-yl)acetic acid dihydrochloride

1) (5-{[5-{[(tert-Butoxycarbonyl)amino]methyl}-6-isobutyl-2-methyl-4-(4-methylphenyl)pyridin-3-yl]methylene}-4-oxo-2-thioxo-1,3-thiazolidin-3-yl)acetic acid (355 mg, yield 50%) was obtained as a yellow powder from tert-butyl {[5-formyl-2-isobutyl-6-methyl-4-(4-methylphenyl)pyridin-3-yl]methyl}carbamate (500 mg, 1.26 mmol) and (4-oxo-2-thioxo-1,3-thiazolidin-3-yl)acetic acid (241 mg, 1.26 mmol) according to a method similar to the method of Example 315-1).

¹H-NMR (CDCl₃) δ: 0.98 (6H, d, J=6.6 Hz), 1.39 (9H, s), 2.09-2.27 (1H, m), 2.36 (3H, s), 2.50 (3H, s), 2.8 (2H, d, J=7.4 Hz), 4.01-4.18 (4H, m), 4.20 (1H, brs), 6.96 (2H, d, J=7.9 Hz), 7.20 (2H, d, J=7.9 Hz), 7.38 (1H, s).

2) (5-{[5-(Aminomethyl)-6-isobutyl-2-methyl-4-(4-methylphenyl)pyridin-3-yl]methylene}-4-oxo-2-thioxo-1,3-thiazolidin-3-yl)acetic acid dihydrochloride (198 mg, yield 100%) was obtained as a yellow powder from (5-{[5-{[(tert-butoxycarbonyl)amino]methyl}-6-isobutyl-2-methyl-4-(4-methylphenyl)pyridin-3-yl]methylene}-4-oxo-2-thioxo-1,3-thiazolidin-3-yl)acetic acid (210 mg, 0.386 mmol) according to a method similar to the method of Example 2-3).

¹H-NMR (DMSO-d₆)δ:0.98 (6H, d, J=6.4 Hz), 2.17-2.31 (1H, m), 2.36 (3H, s), 2.55 (3H, s), 2.95 (2H, d, J=6.6 Hz), 3.80 (2H, d, J=7.4 Hz), 4.63 (2H, s), 7.22 (2H, d, J=8.1 Hz), 7.30 (2H, d, J=8.1 Hz), 7.55 (1H, s), 8.35 (3H, brs).

### Example 335

### 5-{[5-(aminomethyl)-6-isobutyl-2-methyl-4-(4-methylphenyl)pyridin-3-yl]methylene}-2-thioxo-1,3-thiazolidin-4-one dihydrochloride

1) tert-Butyl ({2-isobutyl-6-methyl-4-(4-methylphenyl)-5-[(4-oxo-2-thioxo-1,3-thiazolidine-5-ylidene)methyl]pyridin-3-yl}methyl)carbamate (310 mg, yield 48%) was obtained as a yellow powder from tert-butyl {[5-formyl-2-isobutyl-6-methyl-4-(4-methylphenyl)pyridin-3-yl]methyl}carbamate (500 mg, 1.26 mmol) and 2-thioxo-1,3-thiazolidin-4-one (168 mg, 1.26 mmol) according to a method similar to the method of Example 315-1).

¹H-NMR (CDCl₃) δ: 0.98 (6H, d, J=6.8 Hz), 1.39 (9H, s), 2.15-2.31 (1H, m), 2.37 (3H, s), 2.50 (3H, s), 2.80 (2H, d, J=7.4 Hz), 4.13 (2H, d, J=7.4 Hz), 4.20 (1H, brs), 6.95 (2H, d, J=7.7 Hz), 7.20 (2H, d, J=7.7 Hz), 7.34 (1H, s).

2) 5-{[5-(Aminomethyl)-6-isobutyl-2-methyl-4-(4-methylphenyl)pyridin-3-yl]methylene}-2-thioxo-1,3-thiazolidin-4-one dihydrochloride (173 mg, yield 100%) was obtained as a yellow powder from tert-butyl ({2-isobutyl-6-methyl-4-(4-methylphenyl)-5-[(4-oxo-2-thioxo-1,3-thiazolidin-5-ylidene)methyl]pyridin-3-yl}methyl)carbamate (200 mg, 0.390 mmol) according to a method similar to the method of Example 2-3).

¹H-NMR (DMSO-d₆) δ:0.97 (6H, d, J=6.6 Hz), 2.11-2.31 (1H, m), 2.36 (3H, s), 2.52 (2H, s), 2.90 (3H, s), 3.79 (2H, s), 7.19 (2H, d, J=8.1 Hz), 7.26-7.37 (3H, m), 8.27 (3H, brs).

### Example 336

### methyl 3-({[5-(aminomethyl)-6-isobutyl-2-methyl-4-(4-methylphenyl)pyridin-3-yl]acetyl}amino)benzoate dihydrochloride

1) Methyl 3-({[5-{[(tert-Butoxycarbonyl)amino]methyl}-6-isobutyl-2-methyl-4-(4-methylphenyl)pyridin-3-yl]acetyl}amino)benzoate (230 mg, yield 35%) was obtained as a white powder from [5-{[(tert-butoxycarbonyl)amino]methyl}-6-isobutyl-2-methyl-4-(4-methylphenyl)pyridin-3-yl]acetic acid (500 mg, 1.17 mmol) and methyl 3-aminobenzoate (532 mg, 3.52 mmol) according to a method similar to the method of Example 311-1).

¹H-NMR (CDCl₃) δ: 0.98 (6H, d, J=6.6 Hz), 1.38 (9H, s), 2.16-2.31 (1H, m), 2.41 (3H, s), 2.64 (3H, s), 2.77 (2H, d, J=7.4 Hz), 3.47 (2H, s), 3.91 (3H, s), 4.07 (2H, d, J=4.5 Hz), 4.20 (1H, brs), 5.50 (1H, brs), 7.02 (2H, d, J=7.9 Hz), 7.24 (2H, d, J=7.9 Hz), 7.38 (1H, t, J=7.9 Hz), 7.72-7.86 (3H, m).

2) Methyl 3-({[5-(Aminomethyl)-6-isobutyl-2-methyl-4-(4-methylphenyl)pyridin-3-yl]acetyl}amino)benzoate dihydrochloride (65 mg, yield 91%) was obtained as a white powder from methyl 3-({[5-{[(tert-butoxycarbonyl)amino]methyl}-6-isobutyl-2-methyl-4-(4-methylphenyl)pyridin-3-yl]acetyl}amino)benzoate (75.2 mg, 0.134 mmol) according to a method similar to the method of Example 2-3).

¹H-NMR (DMSO-d₆)δ:0.98 (6H, d, J=6.6 Hz), 2.11-2.30 (1H, m), 2.36 (3H, s), 2.53 (3H, s), 2.68 (2H, s), 2.98 (2H, s), 3.78 (2H, s), 3.84 (3H, s), 7.19 (2H, d, J=8.1 Hz), 7.32 (2H, d, J=8.1 Hz), 7.44 (1H, t, J=7.9 Hz), 7.61-7.71 (2H, m), 8.10 (3H, brs), 8.20 (1H, s), 10.6 (1H, brs).

### Example 337

### methyl 3-({[5-(aminomethyl)-6-isobutyl-2-methyl-4-(4-methylphenyl)pyridin-3-yl]methyl}thio)pyridine-2-carboxylate trihydrochloride

1) Methyl 3-({[5-{[(tert-butoxycarbonyl)amino]methyl}-6-isobutyl-2-methyl-4-(4-methylphenyl)pyridin-3-yl]methyl}thio)pyridine-2-carboxylate (1.43 g, 2.60 mmol) was obtained as a yellow oil from tert-butyl {[5-(hydroxymethyl)-2-isobutyl-6-methyl-4-(4-methylphenyl)pyridin-3-yl]methyl}carbamate (2.08 y, 5.22 mmol) and methyl 3-mercaptopyridine-2-carboxylate (883 mg, 5.22 mmol) according to a method similar to the method of Example 183-1).

¹H-NMR (CDCl₃) δ:0.98 (6H, d, J=6.6 Hz), 1.39 (9H, s), 2.14-2.26 (1H, m), 2.35 (3H, s), 2.66 (3H, s), 2.76 (2H, d, J=7.2 Hz), 3.76 (2H, s), 3.99 (3H, s), 4.03 (2H, d, J=5.3 Hz), 4.19 (1H, brs), 7.04-7.07 (1H, m), 7.09 (2H, d, J=8.1 Hz), 7.18 (2H, d, J=7.7 Hz), 7.28-7.31 (1H, m), 7.40-7.44 (1H, m), 8.43 (1H, dd, J=4.5, 1.5 Hz).

2) Methyl 3-({[5-(aminomethyl)-6-isobutyl-2-methyl-4-(4-methylphenyl)pyridin-3-yl]methyl}thio)pyridine-2-carboxylate trihydrochloride (161 mg, yield 80%) was obtained as a pale-yellow solid from methyl 3-({[5-{[(tert-butoxycarbonyl)amino]methyl}-6-isobutyl-2-methyl-4-(4-methylphenyl)pyridin-3-yl]methyl}thio)pyridine-2-carboxylate (197 mg, 0.359 mmol) according to a method similar to the method of Example 2-3).

¹H-NMR (DMSO-d₆) δ:1.00 (6H, d, J=6.4 Hz), 2.15-2.26 (1H, m), 2.35 (3H, s), 2.89 (3H, brs), 3.18 (2H, brs), 3.77 (2H, d, J=5.1 Hz), 3.83 (3H, s), 3.94 (2H, s), 7.25 (2H, d, J=7.9 Hz), 7.31 (2H, d, J=8.1 Hz), 7.51 (1H, dd, J=8.3, 4,5 Hz), 7.76 (1H, d, J=8.1 Hz), 8.35-8.53 (4H, m).

### Example 338

### 3-({[5-(aminomethyl)-6-isobutyl-2-methyl-4-(4-methylphenyl)pyridin-3-yl]methyl}thio)pyridine-2-carboxylic acid trihydrochloride

1) 3-({[5-{[(tert-Butoxycarbonyl)amino]methyl}-6-isobutyl-2-methyl-4-(4-methylphenyl)pyridin-3-yl]methyl}thio)pyridine-2-carboxylic acid (1.19 g, yield 99%) was obtained as a colorless oil from methyl 3-({[5-{[(tert-butoxycarbonyl)amino]methyl}-6-isobutyl-2-methyl-4-(4-methylphenyl)pyridin-3-yl]methyl}thio)pyridine-2-carboxylate (1.23 g, 2.24 mmol) according to a method similar to the method of Example 9-1).

¹H-NMR (CDCl₃) δ:1.06 (6H, d, J=6.6 Hz), 1.38 (9H, s), 2.21-2.32 (1H, m), 2.37 (3H, s), 2.97 (3H, brs), 3.17 (2H, brs), 3.81 (2H, s), 4.08-4.13 (2H, m), 4.31 (1H, brs), 7.14 (2H, d, J=7.9 Hz), 7.24 (2H, d, J=8.3 Hz), 7.42-7.46 (1H, m), 7.50-7.53 (1H, m), 8.35 (1H, dd, J=4.4, 1.2 Hz).

2) 3-({[5-(Aminomethyl)-6-isobutyl-2-methyl-4-(4-methylphenyl)pyridin-3-yl]methyl}thio)pyridine-2-carboxylic acid trihydrochloride (265 mg, yield 69%) was obtained as a pale-yellow solid from 3-({[5-{[(tert-butoxycarbonyl)amino]methyl}-6-isobutyl-2-methyl-4-(4-methylphenyl)pyridin-3-yl]methyl}thio)pyridine-2-carboxylic acid (0.38 g, 0.709 mmol) according to a method similar to the method of Example 2-3).

¹H-NMR (DMSO-d₆) δ:0.99 (6H, d, J=6.6 Hz), 2.13-2.24 (1H, m), 2.34 (3H, s), 2.79-2.82 (3H, m), 3.05 (2H, brs), 3.75 (2H, brs), 3.89 (2H, brs), 7.26 (2H, d, J=6.4 Hz), 7.31 (2H, d, J=8.3 Hz), 7.48 (1H, dd, J=8.3, 4.5 Hz), 7.72 (1H, d, J=8.3 Hz), 8.19-8.36 (3H, m), 8.43 (1H, d, J=4.5 Hz).

### Example 339

### 3-({[5-(aminomethyl)-6-isobutyl-2-methyl-4-(4-methylphenyl)pyridin-3-yl]methyl}thio)pyridine-2-carboxamide trihydrochloride

1) tert-Butyl {[5-({[2-(aminocarbonyl)pyridin-3-yl]thio}methyl)-2-isobutyl-6-methyl-4-(4-methylphenyl)pyridin-3-yl]methyl}carbamate (720 mg, yield 88%) was obtained as a colorless oil from 3-({[5-{[(tert-butoxycarbonyl)amino]methyl}-6-isobutyl-2-methyl-4-(4-methylphenyl)pyridin-3-yl]methyl}thio)pyridine-2-carboxylic acid (0.82 g, 1.53 mmol) according to a method similar to the method of Example 3-1).

¹H-NMR (CDCl₃) δ:0.98 (6H, d, J=6.6 Hz), 1.38 (9H, s), 2.14-2.26 (1H, m), 2.33 (3H, s), 2.67 (3H, s), 2.75 (2H, d, J=7.2 Hz), 3.71 (2H, s), 4.03 (2H, d, J=4.9 Hz), 4.18 (1H, brs), 5.44 (1H, brs), 7.12-7.18 (4H, m), 7.25-7.29 (1H, m), 7.42 (1H, dd, J=8.3, 1.3 Hz), 7.82 (1H, brs), 8.24 (1H, dd, J=4.3, 1.3 Hz).

2) 3-({[5-(Aminomethyl)-6-isobutyl-2-methyl-4-(4-methylphenyl)pyridin-3-yl]methyl}thio)pyridine-2-carboxamide trihydrochloride (546 mg, yield 74%) was obtained as a pale-yellow solid from tert-butyl {[5-({[2-(aminocarbonyl)pyridin-3-yl]thio}methyl)-2-isobutyl-6-methyl-4-(4-methylphenyl)pyridin-3-yl]methyl}carbamate (720 mg, 1.35 mmol) according to a method similar to the method of Example 2-3).

¹H-NMR (DMSO-d₆) δ:1.01 (6H, d, J=6.6 Hz), 2.13-2.26 (1H, m), 2.34 (3H, s), 2.96 (3H, s), 3.25 (2H, brs), 3.79 (2H, d, J=5.1 Hz), 3.86 (2H, s), 7.29-7.40 (4H, m), 7.46 (1H, dd, J=8.1, 4.5 Hz), 7.64 (1H, brs), 7.69 (1H, d, J=7.5 Hz), 8.09 (1H, brs), 8.36 (1H, dd, J=4.5, 1.2 Hz), 8.51 (3H, brs).

### Example 340

### 4-[({[5-(aminomethyl)-6-isobutyl-2-methyl-4-(4-methylphenyl)pyridin-3-yl]carbonyl}oxy)methyl]cyclohexanecarboxylic acid dihydrochloride

1) A mixture of methyl 4-(hydroxymethyl)cyclohexanecarboxylate (0.40 g, 2.32 mmol), triethylamine (0.65 mL, 4.64 mmol) and tetrahydrofuran (10 mL) was cooled to 0° C. and methanesulfonyl chloride (0.27 mL, 3.48 mmol) was added dropwise. After stirring at room temperature for 30 min., the reaction mixture was poured into saturated aqueous sodium hydrogen carbonate, and the mixture was extracted with ethyl acetate. The extract was dried over anhydrous magnesium sulfate and the solvent was evaporated under reduced pressure to give methyl 4-{[(methylsulfonyl)oxy]methyl}cyclohexanecarboxylate as a crude product. The crude product was dissolved in N,N-dimethylformamide (15 mL), and potassium carbonate (480 mg, 3.48 mmol) and 5-{[(tert-butoxycarbonyl)amino]methyl}-6-isobutyl-2-methyl-4-(4-methylphenyl)nicotinic acid (0.95 g, 2.32 mmol) were added. The mixture was stirred with heating at 70° C. for 1 hr. The reaction mixture was diluted with ethyl acetate, washed with saturated brine and dried over anhydrous magnesium sulfate. The solvent was evaporated under reduced pressure and the obtained residue was purified by silica gel column chromatography to give [4-(methoxycarbonyl)cyclohexyl]methyl 5-{[(tert-butoxycarbonyl)amino]methyl}-6-isobutyl-2-methyl-4-(4-methylphenyl)nicotinate (750 mg, yield 57%) as a colorless oil.

¹H-NMR (CDCl₃) δ:0.97 (6H, d, J=6.6 Hz), 1.07-1.18 (2H, m), 1.33-1.49 (14H, m), 1.83-1.96 (2H, m), 2.16-2.25 (1H, m), 2.39 (3H, s), 2.48-2.56 (4H, m), 2.78 (2H, d, J=7.4 Hz), 3.67 (3H, s), 3.78 (2H, d, J=6.8 Hz), 4.13-4.17 (2H, m), 4.23 (1H, brs), 7.07 (2H, d, J=7.9 Hz), 7.20 (2H, d, J=7.7 Hz).

2) 4-[({[5-{[(tert-Butoxycarbonyl)amino]methyl}-6-isobutyl-2-methyl-4-(4-methylphenyl)pyridin-3-yl]carbonyl}oxy)methyl]cyclohexanecarboxylic acid (550 mg, yield 75%) was obtained as a white solid from [4-(methoxycarbonyl)cyclohexyl]methyl 5-{[(tert-butoxycarbonyl)amino]methyl}-6-isobutyl-2-methyl-4-(4-methylphenyl)nicotinate (750 mg, 1.32 mmol) according to a method similar to the method of Example 9-1).

¹H-NMR (CDCl₃) δ:0.97 (6H, d, J=6.6 Hz), 1.08-1.20 (2H, m), 1.33-1.68 (14H, m), 1.86-1.96 (2H, m), 2.15-2.28 (1H, m), 2.38 (3H, s), 2.54-2.60 (4H, m), 2.78 (2H, brs), 3.78 (2H, d, J=6.6 Hz), 4.12-4.16 (2H, m), 4.24 (1H, brs), 7.07 (2H, d, J=7.9 Hz), 7.20 (2H, d, J=7.7 Hz).

3) 4-[({[5-(Aminomethyl)-6-isobutyl-2-methyl-4-(4-methylphenyl)pyridin-3-yl]carbonyl}oxy)methyl]cyclohexanecarboxylic acid dihydrochloride (254 mg, yield 83%) was obtained as a white solid from 4-[({[5-{[(tert-butoxycarbonyl)amino]methyl}-6-isobutyl-2-methyl-4-(4-methylphenyl)pyridin-3-yl]carbonyl}oxy)methyl]cyclohexanecarboxylic acid (320 mg, 0.579 mmol) according to a method similar to the method of Example 2-3).

¹H-NMR (DMSO-d₆) δ:0.97 (6H, d, J=6.6 Hz), 1.17-1.42 (7H, m), 1.66 1.82 (2H, m), 2.14-2.24 (1H, m), 2.37 (3H, s), 2.41-2.45 (1H, m), 2.54 (3H, s), 2.86-2.97 (2H, m), 3.76 (2H, d, J=6.6 Hz), 3.83 (2H, d, J=4.7 Hz), 7.20 (2H, d, J=7.9 Hz), 7.30 (2H, d, J=8.1 Hz), 8.34 (3H, brs).

### Example 341

### N-[5-(aminomethyl)-6-isobutyl-2-methyl-4-(4-methylphenyl)pyridin-3-yl]thiophene-2-carboxamide dihydrochloride

N-[5-(Aminomethyl)-6-isobutyl-2-methyl-4-(4-methylphenyl)pyridin-3-yl]thiophene-2-carboxamide dihydrochloride (171 mg, yield 75%) was obtained as a white powder from tert-butyl {[5-amino-2-isobutyl-6-methyl-4-(4-methylphenyl)pyridin-3-yl]methyl}carbamate (192 mg, 0.5 mmol) and thiophene-2-carbonyl chloride (110 mg, 0.75 mmol) according to a method similar to the method of Example 223.

¹H-NMR (DMSO-d₆) δ: 1.00 (6H, d, J=6.6 Hz), 2.20-2.31 (1H, m), 2.31 (3H, s), 2.63 (3H, s), 3.07 (2H, brs), 3.86 (2H, s), 7.12 (1H, dd, J=3.3, 4.8 Hz), 7.25 (4H, s), 7.74 (1H, d, J=3.3 Hz), 7.79 (1H, d, J=4.8 Hz), 8.42 (3H, brs), 10.18 (1H, brs).

### Example 342

### 3-({[5-(aminomethyl)-6-isobutyl-2-methyl-4-(4-methylphenyl)pyridin-3-yl]acetyl}amino)benzoic acid dihydrochloride

1) 3-({[5-{[(tert-Butoxycarbonyl)amino]methyl}-6-isobutyl-2-methyl-4-(4-methylphenyl)pyridin-3-yl]acetyl}amino)benzoic acid (110 mg, yield 87%) was obtained as a white powder from methyl 3-({[5-{[(tert-butoxycarbonyl)amino]methyl}-6-isobutyl-2-methyl-4-(4-methylphenyl)pyridin-3-yl]acetyl}amino)benzoate (130 mg, 0.232 mmol) according to a method similar to the method of Example 9-1).

¹H-NMR (CDCl₃) δ: 0.94 (6H, d, J=6.6 Hz), 1.38 (9H, s), 2.10-2.27 (1H, m), 2.36 (3H, s), 2.89-3.10 (5H, m), 3.90 (2H, d, J=5.7 Hz), 4.10 (2H, d, J=7.2 Hz), 4.20 (1H, brs), 4.90 (1H, brs), 7.13 (2H, d, J=8.1 Hz), 7.24 (2H, d, J=8.1 Hz), 7.32 (1H, t, J=8.0 Hz), 7.65 (1H, d J=7.7 Hz), 7.89 (1H, s), 8.17 (1H, s).

2) 3-({[5-(Aminomethyl)-6-isobutyl-2-methyl-4-(4-methylphenyl)pyridin-3-yl]acetyl}amino)benzoic acid dihydrochloride (95 mg, yield 95%) was obtained as a white powder from 3-({[5-{[(tert-butoxycarbonyl)amino]methyl}-6-isobutyl-2-methyl-4-(4-methylphenyl)pyridin-3-yl]acetyl}amino)benzoic acid (105 mg, 0.192 mmol) according to a method similar to the method of Example 2-3).

¹H-NMR (DMSO-d₆) δ:1.00 (6H, d, J=6.8 Hz), 2.08-2.25 (1H, m), 2.37 (3H, s), 2.51 (3H, s), 2.83 (2H, s), 3.20 (2H, s), 3.82 (2H, s), 7.09-7.51 (5H, m), 7.54-7.79 (2H, m), 8.14 (1H, s), 8.44 (3H, s), 10.34 (1H, brs).

### Example 343

### methyl 4-[({[5-(aminomethyl)-6-isobutyl-2-methyl-4-(4-methylphenyl)pyridin-3-yl]acetyl}amino)methyl]benzoate dihydrochloride

1) Methyl 4-[({[5-{[(tert-butoxycarbonyl)amino]methyl}-6-isobutyl-2-methyl-4-(4-methylphenyl)pyridin-3-yl]acetyl}amino)methyl]benzoate (350 mg, yield 67%) was obtained as a white powder from [5-{[(tert-butoxycarbonyl)amino]methyl}-6-isobutyl-2-methyl-4-(4-methylphenyl)pyridin-3-yl]acetic acid (390 mg, 0.914 mmol) and methyl 4-(aminomethyl)benzoate (553 mg, 2.74 mmol) according to a method similar to the method of Example 311-1).

¹H-NMR (CDCl₃) δ: 0.96 (6H, d, J=6.6 Hz), 1.37 (9H, s), 2.11-2.29 (1H, m), 2.39 (3H, s), 2.55 (3H, s), 2.74 (2H, d, J=7.2 Hz), 3.35 (2H, s), 3.93 (3H, s), 4.02 (2H, d, J=5.1 Hz), 4.20 (1H, brs), 4.39 (2H, d, J=5.8 Hz), 5.49 (1H, brs), 6.90 (2H, d, J=7.9 Hz), 7.16 (2H, d, J=7.9 Hz), 7.23 (2H, d, J=8.1 Hz), 7.99 (2H, d, J=8.1 Hz).

2) Methyl 4-[({[5-(aminomethyl)-6-isobutyl-2-methyl-4-(4-methylphenyl)pyridin-3-yl]acetyl}amino)methyl]benzoate dihydrochloride (51 mg, yield 89%) was obtained as a white powder from methyl 4-[({[5-{[(tert-butoxycarbonyl)amino]methyl}-6-isobutyl-2-methyl-4-(4-methylphenyl)pyridin-3-yl]acetyl}amino)methyl]benzoate (60 mg, 0.105 mmol) according to a method similar to the method of Example 2-3).

¹H-NMR (DMSO-d₆) δ:0.99 (6H, d, J=6.6 Hz), 2.11-2.27 (1H, m), 2.40 (3H, s), 2.81 (3H, s), 3.24 (2H, d, J=6.0 Hz), 3.44 (2H, s), 3.78-3.89 (5H, m), 4.28 (2H, d, J=5.5 Hz), 7.20 (2H, d, J=7.9 Hz), 7.27-7.38 (5H, m),7.94 (2H, d, J=7.9 Hz), 8.54 (3H, brs).

### Example 344

### 5-[({[5-(aminomethyl)-6-isobutyl-2-methyl-4-(4-methylphenyl)pyridin-3-yl]carbonyl}oxy)methyl]pyrazine-2-carboxylic acid dihydrochloride

1) Methyl 5-[({[5-{[(tert-butoxycarbonyl)amino]methyl}-6-isobutyl-2-methyl-4-(4-methylphenyl)pyridin-3-yl]carbonyl}oxy)methyl]pyrazine-2-carboxylate (1.35 g, yield 98%) was obtained as a colorless oil from 5-{[(tert-butoxycarbonyl)amino]methyl}-6-isobutyl-2-methyl-4-(4-methylphenyl)nicotinic acid (1.00 g, 2.43 mmol) and methyl 5-(bromomethyl)pyrazine-2-carboxylate (0.51 g, 2.21 mmol) according to a method similar to the method of Example 169-1).

¹H-NMR (CDCl₃) δ:0.97 (6H, d, J=6.6 Hz), 1.39 (9H, s), 2.17-2.27 (1H, m), 2.31 (3H, s), 2.58 (3H, s), 2.79 (2H, d, J=7.2 Hz), 4.06 (3H, s), 4.12-4.16 (2H, m), 4.22 (1H, brs), 5.13 (2H, s), 7.02 (2H, d, J=8.1 Hz), 7.10 (2H, d, J=7.9 Hz), 8.36 (1H, d, J=1.3 Hz), 9.19 (1H, d, J=1.3 Hz).

2) 5-[({[5-{[(tert-Butoxycarbonyl)amino]methyl}-6-isobutyl-2-methyl-4-(4-methylphenyl)pyridin-3-yl]carbonyl}oxy)methyl]pyrazine-2-carboxylic acid (600 mg, yield 45%) was obtained as a colorless oil from methyl 5-[({[5-{[(tert-butoxycarbonyl)amino]methyl}-6-isobutyl-2-methyl-4-(4-methylphenyl)pyridin-3-yl]carbonyl}oxy)methyl]pyrazine-2-carboxylate (1.35 g, 2.40 mmol) according to a method similar to the method of Example 9-1).

¹H-NMR (CDCl₃) δ:0.97 (6H, d, J=6.8 Hz), 1.39 (9H, s), 2.16-2.28 (1H, m), 2.33 (3H, s), 2.59 (3H, s), 2.82 (2H, d, J=7.4 Hz), 4.11-4.19 (2H, m), 4.24 (1H, brs), 5.18 (2H, s), 7.04 (2H, d, J=7.9 Hz), 7.12 (2H, d, J=7.2 Hz), 8.20 (1H, s), 9.30 (1H, s).

3) 5-[({[5-(Aminomethyl)-6-isobutyl-2-methyl-4-(4-methylphenyl)pyridin-3-yl]carbonyl}oxy)methyl]pyrazine-2-carboxylic acid dihydrochloride (497 mg, yield 76%) was obtained as a yellow solid from 5-[({[5-{[(tert-butoxycarbonyl)amino]methyl}-6-isobutyl-2-methyl-4-(4-methylphenyl)pyridin-3-yl]carbonyl}oxy)methyl]pyrazine-2-carboxylic acid (600 mg, 1.09 mmol) according to a method similar to the method of Example 2-3).

¹H-NMR (DMSO-d₆) δ:0.97 (6H, d, J=6.6 Hz), 2.17-2.26 (1H, m), 2.29 (3H, s), 2.62 (3H, brs), 2.94 (2H, brs), 3.80 (2H, d, J=4.7 Hz), 5.23 (2H, s), 7.08-7.18 (4H, m), 8.38 (3H, brs), 8.43 (1H, d, J=1.3 Hz), 9.10 (1H, d, J=1.3 Hz).

### Example 345

### 4-bromobenzyl 5-(aminomethyl)-6-isobutyl-2-methyl-4-(4-methylphenyl)nicotinate dihydrochloride

4-Bromobenzyl 5-(aminomethyl)-6-isobutyl-2-methyl-4-(4-methylphenyl)nicotinate dihydrochloride (628 mg, yield 90%) was obtained as a white solid from 4-bromobenzyl 5-{[(tert-butoxycarbonyl)amino]methyl}-6-isobutyl-2-methyl-4-(4-methylphenyl)nicotinate (0.73 g, 1.26 mmol) according to a method similar to the method of Example 2-3).

¹H-NMR (DMSO-d₆) δ:0.96 (6H, d, J=6.8 Hz), 2.14-2.27 (1H, m), 2.36 (3H, s), 2.87 (2H, brs), 3.80 (2H, d, J=5.3 Hz), 4.97 (2H, s), 7.00 (2H, d, J=8.5 Hz), 7.12 (2H, d, J=8.1 Hz), 7.19 (2H, d, J=8.1 Hz), 7.50 (2H, d, J=8.5 Hz), 8.26 (3H, brs).

### Example 346

### {[5[(2-bromophenoxy)methyl[-2-isobutyl-6-methyl-4-(4-methylphenyl)pyridin-3-yl]methyl}amine dihydrochloride

1) tert-Butyl {[5-[(2-bromophenoxy)methyl]-2-isobutyl-6-methyl-4-(4-methylphenyl)pyridin-3-yl]methyl}carbamate (640 mg, yield 46%) was obtained as a white solid from tert-butyl {[5-(hydroxymethyl)-2-isobutyl-6-methyl-4-(4-methylphenyl)pyridin-3-yl]methyl}carbamate (1.00 g, 2.51 mmol) and 2-bromophenol (478 mg, 2.76 mmol) according to a method similar to the method of Example 214-1).

¹H-NMR (CDCl₃) δ:0.99 (6H, d, J=6.6 Hz), 1.39 (9H, s), 2.19-2.28 (1H, m), 2.37 (3H, s), 2.69 (3H, s), 2.79 (2H, d, J=7.4 Hz), 4.08-4.11 (2H, m), 4.24 (1H, brs), 4.67 (2H, s), 6.65 (1H, dd, J=8.1, 1.3 Hz), 6.79-6.84 (1H, m), 7.07 (2H, d, J=8.1 Hz), 7.12-7.19 (3H, m), 7.51 (1H, dd, J=7.9, 1.5 Hz).

2) {[5-[(2-Bromophenoxy)methyl]-2-isobutyl-6-methyl-4-(4-methylphenyl)pyridin-3-yl]methyl}amine dihydrochloride (458 mg, yield 75%) was obtained as a white solid from tert-butyl {[5-[(2-bromophenoxy)methyl]-2-isobutyl-6-methyl-4-(4-methylphenyl)pyridin-3-yl]methyl}carbamate (640 mg, 1.16 mmol) according to a method similar to the method of Example 2-3).

¹H-NMR (DMSO-d₆) δ:1.01 (6H, d, J=6.6 Hz), 2.16-2.30 (1H, m), 2.36 (3H, s), 2.91 (3H, brs), 3.20 (2H, brs), 3.79-3.90 (2H, m), 4.79 (2H, s), 6.89-6.95 (2H, m), 7.25-7.36 (5H, m), 7.58 (1H, dd, J=7.7, 1.5 Hz), 8.48 (3H, brs).

### Example 347

### 4-[({[5-(aminomethyl)-6-isobutyl-2-methyl-4-(4-methylphenyl)pyridin-3-yl]carbonyl}oxy)methyl]-3-methoxybenzoic acid dihydrochloride

1) 2-Methoxy-4-(methoxycarbonyl)benzyl 5-{[(tert-butoxycarbonyl)amino]methyl}-6-isobutyl-2-methyl-4-(4-methylphenyl)nicotinate (1.15 g, yield 100%) was obtained as a colorless oil from 5-{[(tert-butoxycarbonyl)amino]methyl}-6-isobutyl-2-methyl-4-(4-methylphenyl)nicotinic acid (0.80 g, 1.94 mmol) and methyl 4-(bromomethyl)-3-methoxybenzoate (503 mg, 1.94 mmol) according to a method similar to the method of Example 169-1).

¹H-NMR (CDCl₃) δ:0.96 (6H, d, J=6.6 Hz), 1.38 (9H, s), 2.15-2.24 (1H, m), 2.34 (3H, s), 2.54 (3H, s), 2.77 (2H, d, J=7.2 Hz), 3.85 (3H, s), 3.93 (3H, s), 4.10-4.16 (2H, m), 4.20 (1H, brs), 5.06 (2H, s), 6.96 (1H, d, J=7.9 Hz), 7.03 (2H, d, J=8.1 Hz), 7.10 (2H, d, J=7.9 Hz), 7.48-7.53 (2H, m).

2) 4-[({[5-{[(tert-Butoxycarbonyl)amino]methyl}-6-isobutyl-2-methyl-4-(4-methylphenyl)pyridin-3-yl]carbonyl}oxy)methyl]-3-methoxybenzoic acid (1.10 g, yield 97%) was obtained as a colorless oil from 2-methoxy-4-(methoxycarbonyl)benzyl 5-{[(tert-butoxycarbonyl)amino]methyl}-6-isobutyl-2-methyl-4-(4-methylphenyl)nicotinate (1.15 g, 1.94 mmol) according to a method similar to the method of Example 9-1).

¹H-NMR (CDCl₃) δ:0.97 (6H, d, J=6.6 Hz), 1.39 (9H, s), 2.16-2.26 (1H, m), 2.35 (3H, s), 2.56 (3H, s), 2.80 (2H, d, J=7.2 Hz), 3.86 (3H, s), 4.11-4.16 (2H, m), 4.23 (1H, brs), 5.08 (2H, s), 6.97 (1H, d, J=7.9 Hz), 7.04 (2H, d, J=7.7 Hz), 7.11 (2H, d, J=7.7 Hz), 7.53 (1H, s), 7.58 (1H, d, J=7.9 Hz).

3) 4-[({[5-(Aminomethyl)-6-isobutyl-2-methyl-4-(4-methylphenyl)pyridin-3-yl]carbonyl}oxy)methyl]-3-methoxybenzoic acid dihydrochloride (247 mg, yield 74%) was obtained as a white solid from 4-[({[5-{[(tert-butoxycarbonyl)amino]methyl}-6-isobutyl-2-methyl-4-(4-methylphenyl)pyridin-3-yl]carbonyl}oxy)methyl]-3-methoxybenzoic acid (0.35 g, 0.607 mmol) according to a method similar to the method of Example 2-3).

¹H-NMR (DMSO-d₆) δ:0.96 (6H, d, J=6.6 Hz), 2.17-2.26 (1H, m), 2.32 (3H, s), 2.84 (2H, brs), 3.79 (2H, d, J=5.7 Hz), 3.83 (3H, s), 5.03 (2H, s), 6.96 (1H, d, J=7.7 Hz), 7.13 (2H, d, J=8.1 Hz), 7.18 (2H, d, J=8.1 Hz), 7.42-7.45 (1H, m), 7.46 (1H, s), 8.19 (3H, brs).

### Example 348

### 4-[({[5-(aminomethyl)-6-isobutyl-2-methyl-4-(4-methylpehyl)pyridin-3-yl]carbonyl}oxy)methyl]-2-methoxybenzoic acid dihydrochloride

1) 3-Methoxy-4-(methoxycarbonyl)benzyl 5-{[(tert-butoxycarbonyl)amino]methyl}-6-isobutyl-2-methyl-4-(4-methylphenyl)nicotinate (680 mg, yield 94%) was obtained as a colorless oil from 5-{[(tert-butoxycarbonyl)amino]methyl}-6-isobutyl-2-methyl-4-(4-methylphenyl)nicotinic acid (0.50 g, 1.22 mmol) and methyl 4-(bromomethyl)-2-methoxybenzoate (315 mg, 1.22 mmol) according to a method similar to the method of Example 169-1).

¹H-NMR (CDCl₃) δ:0.96 (6H, d, J=6.6 Hz), 1.38 (9H, s), 2.16-2.25 (1H, m), 2.33 (3H, s), 2.54 (3H, s), 2.78 (2H, d, J=7.4 Hz), 3.86 (3H, s), 3.90 (3H, s), 4.11-4.13 (2H, m), 4.21 (1H, brs), 4.94 (2H, s), 6.65 (1H, dd, J=8.0, 1.4 Hz), 6.75 (1H, d, J=1.1 Hz), 6.99 (2H, d, J=8.1 Hz), 7.08 (2H, d, J=7.7 Hz), 7.70 (1H, d, J=7.9 Hz).

2) 4-[({[5-{[(tert-Butoxycarbonyl)amino]methyl}-6-isobutyl-2-methyl-4-(4-methylphenyl)pyridin-3-yl]carbonyl}oxy)methyl]-2-methoxybenzoic acid (550 mg, yield 83%) was obtained as a colorless oil from 3-methoxy-4-(methoxycarbonyl)benzyl 5-{[(tert-butoxycarbonyl)amino]methyl}-6-isobutyl-2-methyl-4-(4-methylphenyl)nicotinate (680 mg, 1.15 mmol) according to a method similar to the method of Example 9-1).

¹H-NMR (CDCl₃) δ:0.97 (6H, d, J=6.6 Hz), 1.38 (9H, s), 2.16-2.25 (1H, m), 2.33 (3H, s), 2.54 (3H, s), 2.78 (2H, d, J=7.4 Hz), 4.04 (3H, s), 4.11-4.13 (2H, m), 4.20 (1H, brs), 4.98 (2H, s), 6.77 (1H, d, J=9.4 Hz), 6.84 (1H, s), 6.99 (2H, d, J=8.1 Hz), 7.07 (2H, d, J=7.9 Hz), 8.08 (1H, d, J=7.9 Hz).

3) 4-[({[5-(Aminomethyl)-6-isobutyl-2-methyl-4-(4-methylphenyl)pyridin-3-yl]carbonyl}oxy)methyl]-2-methoxybenzoic acid dihydrochloride (240 mg, yield 85%) was obtained as a white solid from 4-[({[5-{[(tert-butoxycarbonyl)amino]methyl}-6-isobutyl-2-methyl-4-(4-methylphenyl)pyridin-3-yl]carbonyl}oxy)methyl]-2-methoxybenzoic acid (293 mg, 0.509 mmol) according to a method similar to the method of Example 2-3).

¹H-NMR (DMSO-d₆) δ:0.96 (6H, d, J=6.6 Hz), 2.14-2.26 (1H, m), 2.33 (3H, s), 2.58 (3H, brs), 2.93 (2H, brs), 3.78 (3H, s), 3.81 (2H, d, J=4.5 Hz), 5.01 (2H, s), 6.62 (1H, d, J=7.9 Hz), 6.92 (1H, d, J=0.9 Hz), 7.12-7.22 (4H, m), 7.55 (1H, d, J=7.7 Hz), 8.37 (3H, brs).

### Example 349

### 4-[({[5-(aminomethyl)-6-isobutyl-2-methyl-4-(4-methylphenyl)pyridin-3-yl]acetyl}amino)methyl]benzoic acid dihydrochloride

1) 4-[({[5-{[(tert-Butoxycarbonyl)amino]methyl}-6-isobutyl-2-methyl-4-(4-methylphenyl)pyridin-3-yl]acetyl}amino)methyl]benzoic acid (182 mg, yield 94%) was obtained as a white powder from methyl 4-[({[5-{[(tert-butoxycarbonyl)amino]methyl}-6-isobutyl-2-methyl-4-(4-methylphenyl)pyridin-3-yl]acetyl}amino)methyl]benzoate (200 mg, 0.349 mmol) according to a method similar to the method of Example 9-1).

¹H-NMR (CDCl₃) δ: 0.92 (6H, d, J=6.6 Hz), 1.34 (9H, s), 2.10-2.24 (1H, m), 2.35 (3H, s), 2.38 (3H, s), 2.58 (2H, s), 3.22 (2H, s), 3.77 (2H, d, J=3.0 Hz), 4.20 (1H, brs), 4.27 (2H, d, J=5.8 Hz), 6.74 (1H, s), 7.09 (2H, d, J=8.1 Hz), 7.17 (2H, d, J=8.1 Hz), 7.28 (2H, d, J=8.3 Hz), 7.90 (2H, d, J=8.3 Hz), 8.17 (1H, s).

2) 4-[({[5-(Aminomethyl)-6-isobutyl-2-methyl-4-(4-methylphenyl)pyridin-3-yl]acetyl}amino)methyl]benzoic acid dihydrochloride (135 mg, yield 95%) was obtained as a white powder from 4-[({[5-{[(tert-butoxycarbonyl)amino]methyl}-6-isobutyl-2-methyl-4-(4-methylphenyl)pyridin-3-yl]acetyl}amino)methyl]benzoic acid (150 mg, 0.268 mmol) according to a method similar to the method of Example 2-3).

¹H-NMR (DMSO-d₆)δ:0.98 (6H, d, J=6.6 Hz), 2.07-2.24 (1H, m), 2.40 (3H, s), 2.78 (3H, s), 3.10 (2H, s), 3.41 (2H, s), 3.78 (2H, s), 4.27 (2H, d, J=5.7 Hz), 7.16 (2H, d, J=7.9 Hz), 7.26-7.34 (4H, m), 7.92 (2H, d, J=8.3 Hz), 8.33 (3H, brs), 8.45 (1H, brs).

### Example 350

### N-[5-(aminomethyl)-6-isobutyl-2-methyl-4-(4-methylphenyl)pyridin-3-yl]isoxazole-4-carboxamide dihydrochloride

N-[5-(Aminomethyl)-6-isobutyl-2-methyl-4-(4-methylphenyl)pyridin-3-yl]isoxazole-4-carboxamide dihydrochloride (173 mg, yield 76%) was obtained as a white powder from tert-butyl {[5-amino-2-isobutyl-6-methyl-4-(4-methylphenyl)pyridin-3-yl]methyl}carbamate (192 mg, 0.5 mmol) and isoxazole-4-carbonyl chloride (100 mg, 0.75 mmol) according to a method similar to the method of Example 223.

¹H-NMR (DMSO-d₆) δ: 0.99 (6H, d, J=6.6 Hz), 2.20-2.31 (1H, m), 2.53 (3H, s), 2.94 (2H, s), 3.82 (2H, brs), 7.09 (1H, s), 7.20 (2H, d, J=8.1 Hz), 7.25 (2H, d, J=8.1 Hz), 8.28 (3H, brs), 8.73 (1H, brs), 10.59 (1H, brs).

### Example 351

### N-[5-(aminomethyl)-6-isobutyl-2-methyl-4-(4-methylphenyl)pyridin-3-yl]furan-2-carboxamide dihydrochloride

N-[5-(Aminomethyl)-6-isobutyl-2-methyl-4-(4-methylphenyl)pyridin-3-yl]furan-2-carboxamide dihydrochloride (190 mg, yield 85%) was obtained as a white powder from tert-butyl {[5-amino-2-isobutyl-6-methyl-4-(4-methylphenyl)pyridin-3-yl]methyl}carbamate (192 mg, 0.5 mmol) and furan-2-carbonyl chloride (100 mg, 0.75 mmol) according to a method similar to the method of Example 223.

¹H-NMR (DMSO-d₆) δ: 1.00 (6H, d, J=6.6 Hz), 2.09-2.30 (1H, m), 2.32 (3H, s), 2.58 (3H, s), 3.04 (2H, brs), 3.83 (2H, s), 6.61 (1H, dd, J=1.8, 3.3 Hz), 7.14 (1H, d, J=3.3 Hz), 7.21 (2H, d, J=7.8 Hz), 7.25 (2H, d, J=7.8 Hz), 7.84 (1H, s), 8.37 (3H, brs), 9.98 (1H, brs).

### Example 352

### N-[5-(aminomethyl) 6 isobutyl-2-methyl-4-(4-methylphenyl)pyridin-3-yl]-4-methylbenzamide dihydrochloride

N-[5-(Aminomethyl)-6-isobutyl-2-methyl-4-(4-methylphenyl)pyridin-3-yl]-4-methylbenzamide dihydrochloride (211 mg, yield 87%) was obtained as a white powder from tert-butyl {[5-amino-2-isobutyl-6-methyl-4-(4-methylphenyl)pyridin-3-yl]methyl}carbamate (192 mg, 0.5 mmol) and 4-methylbenzoyl chloride (116 mg, 0.75 mmol) according to a method similar to the method of Example 223.

¹H-NMR (DMSO-d₆) δ: 1.00 (6H, d, J=6.6 Hz), 2.22-2.32 (1H, m), 2.31 (3H, s), 2.32 (3H, s), 2.57 (3H, s), 3.01 (2H, brs), 3.84 (2H, s), 7.21-7.27 (6H, m), 7.55 (2H, d, J=8.1 Hz), 8.32 (3H, brs), 9.88 (1H, brs).

### Example 353

### N-[5-(aminomethyl)-6-isobutyl-2-methyl-4-(4-methylphenyl)pyridin-3-yl]-4-tert-butylbenzamide dihydrochloride

N-[5-(Aminomethyl)-6-isobutyl-2-methyl-4-(4-methylphenyl)pyridin-3-yl]-4-tert-butylbenzamide dihydrochloride (211 mg, yield 83%) was obtained as a white powder from tert-butyl {[5-amino-2-isobutyl-6-methyl-4-(4-methylphenyl)pyridin-3-yl]methyl}carbamate (192 mg, 0.5 mmol) and 4-tert-butylbenzoyl chloride (147 mg, 0.75 mmol) according to a method similar to the method of Example 223.

¹H-NMR (DMSO-d₆) δ: 1.00 (6H, d, J=6.6 Hz), 1.27 (9H, s), 2.22-2.31 (1H, m), 2.31 (3H, s), 2.56 (3H, s), 3.01 (2H, brs), 3.84 (2H, s), 7.21-7.26 (4H, m), 7.44 (2H, d, J=8.4 Hz), 7.60 (2H, d, J=8.4 Hz), 8.32 (3H, brs), 9.91 (1H, brs).

### Example 354

### N-[5-(aminomethyl)-6-isobutyl-2-methyl-4-(4-methylphenyl)pyridin-3-yl]-4-chlorobenzamide dihydrochloride

N-[5-(Aminomethyl)-6-isobutyl-2-methyl-4-(4-methylphenyl)pyridin-3-yl]-4-chlorobenzamide dihydrochloride (203 mg, yield 82%) was obtained as a white powder from tert-butyl {[5-amino-2-isobutyl-6-methyl-4-(4-methylphenyl)pyridin-3-yl]methyl}carbamate (192 mg, 0.5 mmol) and 4-chlorobenzoyl chloride (131 mg, 0.75 mmol) according to a method similar to the method of Example 223.

¹H-NMR (DMSO-d₆) δ: 1.00 (6H, d, J=6.6 Hz), 2.20-2.30 (1H, m), 2.31 (3H, s), 2.62 (3H, s), 3.08 (2H, brs), 3.86 (2H, s), 7.25 (4H, s), 7.52 (2H, d, J=8.4 Hz), 7.67 (2H, d, J=8.4 Hz), 8.41 (3H, brs), 10.20 (1H, brs).

### Example 355

### N-[5-(aminomethyl)-6-isobutyl-2-methyl-4-(4-methylphenyl)pyridin-3-yl]-4-cyanobenzamide dihydrochloride

N-[5-(Aminomethyl)-6-isobutyl-2-methyl-4-(4-methylphenyl)pyridin-3-yl]-4-cyanobenzamide dihydrochloride (209 mg, yield 86%) was obtained as a white powder from tert-butyl {[5-amino-2-isobutyl-6-methyl-4-(4-methylphenyl)pyridin-3-yl]methyl}carbamate (192 mg, 0.5 mmol) and 4-cyanobenzoyl chloride (126mg, 0.75 mmol) according to a method similar to the method of Example 223.

¹H-NMR (DMSO-d₆) δ: 1.00 (6H, d, J=6.6 Hz), 2.10-2.31(1H, m), 25 2.31 (3H, s), 2.59 (3H, s), 3.02 (2H, brs), 3.85 (2H, s), 7.24 (4H, s), 7.76 (2H, d, J=8.1 Hz), 7.94 (2H, d, J=8.1 Hz), 8.36(3H, brs), 10.36 (1H, brs).

### Example 356

### N-[5-(aminomethyl)-6-isobutyl-2-methyl-4-(4-methylphenyl)pyridin-3-yl]-4-trifluoromethylbenzamide dihydrochloride

N-[5-(Aminomethyl)-6-isobutyl-2-methyl-4-(4-methylphenyl)pyridin-3-yl]-4-trifluoromethylbenzamide dihydrochloride (209 mg, yield 86%) was obtained as a white powder from tert-butyl {[5-amino-2-isobutyl-6-methyl-4-(4-methylphenyl)pyridin-3-yl]methyl}carbamate (192 mg, 0.5 mmol) and 4-trifluoromethylbenzoyl chloride (156 mg, 0.75 mmol) according to a method similar to the method of Example 223.

¹H-NMR (DMSO-d₆) δ: 1.00 (6H, d, J=6.6 Hz), 2.21-2.32 (1H, m), 2.31 (3H, s), 2.55 (3H, s), 2.96 (2H, brs), 3.83 (2H, s), 7.22 (2H, d, J=7.8 Hz), 7.26 (2H, d, J=7.8 Hz), 7.78 (2H, d, J=7.8 Hz), 7.82 (2H, d, J=7.8 Hz), 8.27 (3H, brs), 10.21 (1H, brs).

### Example 357

### N-[5-(aminomethyl)-6-isobutyl-2-methyl-4-(4-methylphenyl)pyridin-3-yl]furan-3-carboxamide dihydrochloride

N-[5-(Aminomethyl)-6-isobutyl-2-methyl-4-(4-methylphenyl)pyridin-3-yl]furan-3-carboxamide dihydrochloride (190 mg, yield 85%) was obtained as a white powder from tert-butyl {[5-amino-2-isobutyl-6-methyl-4-(4-methylphenyl)pyridin-3-yl]methyl}carbamate (192 mg, 0.5 mmol) and furan-3-carbonyl chloride (100 mg, 0.75 mmol) according to a method similar to the method of Example 223.

¹H-NMR (DMSO-d₆) δ: 0.99 (6H, d, J=6.6 Hz), 2.21-2.32 (1H, m), 2.55 (3H, s), 2.98 (3H, s), 3.82 (2H, brs), 6.74 (1H, s), 7.20 (2H, d, J=7.8 Hz), 7.25 (2H, d, J=7.8 Hz), 7.69 (1H, s), 8.15 (1H, s), 8.30 (3H, brs), 9.74 (1H, brs).

### Example 358

### N-[5-(aminomethyl)-6-isobutyl-2-methyl-4-(4-methylphenyl)pyridin-3-yl]thiophene-3-carboxamide dihydrochloride

N-[5-(Aminomethyl)-6-isobutyl-2-methyl-4-(4-methylphenyl)pyridin-3-yl]thiophene-3-carboxamide dihydrochloride (233 mg, yield 99%) was obtained as a white powder from tert-butyl {[5-amino-2-isobutyl-6-methyl-4-(4-methylphenyl)pyridin-3-yl]methyl}carbamate (192 mg, 0.5 mmol) and thiophene-3-carbonyl chloride (110 mg, 0.75 mmol) according to a method similar to the method of Example 223.

¹H-NMR (DMSO-d₆) δ: 0.99 (6H, d, J=6.6 Hz), 2.20-2.31 (1H, m), 2.31 (3H, s), 2.59 (3H, s), 3.05 (2H, brs), 3.84 (2H, s), 7.24 (4H, s), 7.36 (1H, dd, J=1.2, 5.1 Hz), 7.56 (1H, dd, J=5.1, 2.7 Hz), 8.10 (1H, d, J=2.7 Hz), 8.35 (3H, brs), 9.91 (1H, brs)

### Example 359

### 4-[({[5-(aminomethyl)-6-isobutyl-2-methyl-4-(4-methylphenyl)pyridin-3-yl]carbonyl}oxy)methyl]-3-fluorobenzoic acid dihydrochloride

1) 2-Fluoro-4-(methoxycarbonyl)benzyl 5-{[(tert-butoxycarbonyl)amino]methyl}-6-isobutyl-2-methyl-4-(4-methylphenyl)nicotinate (650 mg, yield 92%) was obtained as a colorless oil from 5-{[(tert-butoxycarbonyl)amino]methyl}-6-isobutyl-2-methyl-4-(4-methylphenyl)nicotinic acid (0.50 g, 1.21 mmol) and methyl 4-(bromomethyl)-3-fluorobenzoate (299 mg, 1.21 mmol) according to a method similar to the method of Example 169-1).

¹H-NMR (CDCl₃) δ:0.96 (6H, d, J=6.8 Hz), 1.38 (9H, s), 2.16-2.25 (1H, m), 2.33 (3H, s), 2.54 (3H, s), 2.77 (2H, d, J=7.4 Hz), 3.94 (3H, s), 4.09-4.13 (2H, m), 4.20 (1H, brs), 5.05 (2H, s), 6.98-7.09 (5H, m), 7.64-7.71 (2H, m).

2) 4-[({[5-{[(tert-Butoxycarbonyl)amino]methyl}-6-isobutyl-2-methyl-4-(4-methylphenyl)pyridin-3-yl]carbonyl}oxy)methyl]-3-fluorobenzoic acid (450 mg, yield 71%) was obtained as a colorless oil from 2-fluoro-4-(methoxycarbonyl)benzyl 5-{[(tert-butoxycarbonyl)amino]methyl}-6-isobutyl-2-methyl-4-(4-methylphenyl)nicotinate (650 mg, 1.12 mmol) according to a method similar to the method of Example 9-1).

¹H-NMR (CDCl₃) δ:0.97 (6H, d, J=6.8 Hz), 1.38 (9H, s), 2.13-2.25 (1H, m), 2.33 (3H, s), 2.56 (3H, s), 2.80 (2H, d, J=7.2 Hz), 4.09-4.16 (2H, m), 4.22 (1H, brs), 5.07 (2H, s), 7.00-7.12 (5H, m), 7.70-7.76 (2H, m).

3) 4-[({[5-(Aminomethyl)-6-isobutyl-2-methyl-4-(4-methylphenyl)pyridin-3-yl]carbonyl}oxy)methyl]-3-fluorobenzoic acid dihydrochloride (329 mg, yield 76%) was obtained as a white solid from 4-[({[5-{[(tert-butoxycarbonyl)amino]methyl}-6-isobutyl-2-methyl-4-(4-methylphenyl)pyridin-3-yl]carbonyl}oxy)methyl]-3-fluorobenzoic acid (450 mg, 0.797 mmol) according to a method similar to the method of Example 2-3).

¹H-NMR (DMSO-d₆) δ:0.95 (6H, d, J=6.6 Hz), 2.16-2.23 (1H, m), 2.29 (3H, s), 2.86 (2H, brs), 3.78 (2H, d, J=5.5 Hz), 5.11 (2H, s), 7.07-7.13 (4H, m), 7.18 (1H, t, J=7.6 Hz), 7.60-7.69 (2H, m), 8.23 (3H, brs).

### Example 360

### 4-[({[5-(aminomethyl)-6-isobutyl-2-methyl-4-(4-methylphenyl)pyridin-3-yl]carbonyl}oxy)methyl]-3-chlorobenzoic acid dihydrochloride

1) 2-Chloro-4-(methoxycarbonyl)benzyl 5-{[(tert-butoxycarbonyl)amino]methyl}-6-isobutyl-2-methyl-4-(4-methylphenyl)nicotinate (518 mg, yield 99%) was obtained as a colorless oil from 5-{[(tert-butoxycarbonyl)amino]methyl}-6-isobutyl-2-methyl-4-(4-methylphenyl)nicotinic acid (0.36 g, 0.873 mmol) and methyl 4-(bromomethyl)-3-chlorobenzoate (230 mg, 0.873 mmol) according to a method similar to the method of Example 169-1).

¹H-NMR (CDCl₃) δ:0.97 (6H, d, J=6.8 Hz), 1.38 (9H, s), 2.17-2.26 (1H, m), 2.32 (3H, s), 2.56 (3H, s), 2.78 (2H, d, J=7.4 Hz), 3.94 (3H, s), 4.11-4.13 (2H, m), 4.22 (1H, brs), 5.11 (2H, s), 7.02-7.04 (3H, m), 7.09 (2H, d, J=8.1 Hz), 7.78 (1H, dd, J=8.0, 1.6 Hz), 7.99 (1H, d, J=1.5 Hz).

2) 4-[({[5-{[(tert-Butoxycarbonyl)amino]methyl}-6-isobutyl-2-methyl-4-(4-methylphenyl)pyridin-3-yl]carbonyl}oxy)methyl]-3-chlorobenzoic acid (420 mg, yield 83%) was obtained as a white solid from 2-chloro-4-(methoxycarbonyl)benzyl 5-{[(tert-butoxycarbonyl)amino]methyl}-6-isobutyl-2-methyl-4-(4-methylphenyl)nicotinate (518 mg, 0.870 mmol) according to a method similar to the method of Example 9-1).

¹H-NMR (CDCl₃) δ:0.98 (6H, d, J=6.6 Hz), 1.39 (9H, s), 2.22-2.33 (4H, m), 2.59 (3H, brs), 2.82 (2H, brs), 4.09-4.17 (2H, m), 4.25 (1H, brs), 5.13 (2H, s), 7.01-7.14 (5H, m), 7.83 (1H, dd, J=8.0, 1.6 Hz), 8.04 (1H, d, J=1.5 Hz).

3) 4-[({[5-(Aminomethyl)-6-isobutyl-2-methyl-4-(4-methylphenyl)pyridin-3-yl]carbonyl}methyl]-3-chlorobenzoic acid dihydrochloride (265 mg, yield 66%) was obtained as a white solid from 4-[({[5-{[(tert-butoxycarbonyl)amino]methyl}-6-isobutyl-2-methyl-4-(4-methylphenyl)pyridin-3-yl]carbonyl}oxy)methyl]-3-chlorobenzoic acid (420 mg, 0.722 mmol) according to a method similar to the method of Example 2-3).

¹H-NMR (DMSO-d₆) δ:0.96 (6H, d, J=6.6 Hz), 2.15-2.24 (1H, m), 2.29 (3H, s), 2.54 (3H, s), 2.86 (2H, brs), 3.79 (2H, d, J=5.3 Hz), 5.14 (2H, s), 7.13 (4H, s), 7.16 (1H, d, J=7.9 Hz), 7.78 (1H, dd, J=7.9, 1.5 Hz), 7.90 (1H, d, J=1.5 Hz), 8.25 (3H, brs).

### Example 361

### 4-[({[5-(aminomethyl)-6-isobutyl-2-methyl-4-(4-methylphenyl)pyridin-3-yl]carbonyl}oxy)methyl]isophthalic acid dihydrochloride

1) Dimethyl 4-[({[5-{[(tert-butoxycarbonyl)amino]methyl}-6-isobutyl-2-methyl-4-(4-methylphenyl)pyridin-3-yl]carbonyl}oxy)methyl]isophthalate (1.12 g, yield 99%) was obtained as a colorless oil from 5-{[(tert-butoxycarbonyl)amino]methyl}-6-isobutyl-2-methyl-4-(4-methylphenyl)nicotinic acid (0.75 g, 1.82 mmol) and dimethyl 4-(bromomethyl)isophthalate (522 mg, 1.82 mmol) according to a method similar to the method of Example 169-1).

¹H-NMR (CDCl₃) δ:0.98 (6H, d, J=6.6 Hz), 1.39 (9H, s), 2.15-2.26 (1H, m), 2.35 (3H, s), 2.57 (3H, s), 2.79 (2H, d, J=7.4 Hz), 3.91 (3H, s), 3.96 (3H, s), 4.11-4.16 (2H, m), 4.23 (1H, brs), 5.45 (2H, s), 6.99 (1H, d, J=8.1 Hz), 7.06 (2H, d, J=8.3 Hz), 7.13 (2H, d, J=7.9 Hz), 7.99 (1H, dd, J=8.1, 1.9 Hz), 8.59 (1H, d, J=1.9 Hz).

2) 4-[({[5-{[(tert-Butoxycarbonyl)amino]methyl}-6-isobutyl-2-methyl-4-(4-methylphenyl)pyridin-3-yl]carbonyl}oxy)methyl]isophtalic acid (750 mg, yield 68%) was obtained as a colorless oil from dimethyl 4-[({[5-{[(tert-butoxycarbonyl)amino]methyl}-6-isobutyl-2-methyl-4-(4-methylphenyl)pyridin-3-yl]carbonyl}oxy)methyl]isophthalate (1.12 g, 1.81 mmol) according to a method similar to the method of Example 9-1).

¹H-NMR (CDCl₃) δ:0.97 (6H, d, J=6.4 Hz), 1.38 (9H, s), 2.23-2.35 (4H, m), 2.58 (3H, s), 2.86 (2H, d, J=5.1 Hz), 4.11-4.21 (2H, m), 4.35 (1H, brs), 5.48 (2H, s), 7.01-7.17 (5H, m), 7.96-8.08 (1H, m), 8.64-8.75 (1H, m).

3) 4-[({[5-(Aminomethyl)-6-isobutyl-2-methyl-4-(4-methylphenyl)pyridin-3-yl]carbonyl}oxy)methyl]isophthalic acid dihydrochloride (362 mg, yield 90%) was obtained as a white solid from 4-[({[5-{[(tert-butoxycarbonyl)amino]methyl}-6-isobutyl-2-methyl-4-(4-methylphenyl)pyridin-3-yl]carbonyl}oxy)methyl]isophthalic acid (420 mg, 0.711 mmol) according to a method similar to the method of Example 2-3).

¹H-NMR (DMSO-d₆) δ:0.97 (6H, d, J=6.6 Hz), 2.16-2.27 (1H, m), 2.33 (3H, s), 2.57 (3H, brs), 2.90 (2H, brs), 3.82 (2H, d, J=5.1 Hz), 5.42 (2H, s), 7.01 (1H, d, J=8.1 Hz), 7.19 (2H, d, J=8.7 Hz), 7.23 (2H, d, J=8.3 Hz), 7.97 (1H, dd, J=8.1, 1.9 Hz), 8.31 (3H, brs), 8.42 (1H, d, J=1.9 Hz).

### Example 362

### 2-[5-(aminomethyl)-6-isobutyl-2-methyl-4-(4-methylphenyl)pyridin-3-yl]-N-[4-(dimethylamino)phenyl]acetamide trihydrochloride

1) tert-Butyl {[5-(2-{[4-(dimethylamino)phenyl]amino}-2-oxoethyl)-2-isobutyl-6-methyl-4-(4-methylphenyl)pyridin-3-yl]methyl}carbamate (450 mg, yield 71%) was obtained as a white powder from [5-{[(tert-butoxycarbonyl)amino]methyl}-6-isobutyl-2-methyl-4-(4-methylphenyl)pyridin-3-yl]acetic acid (500 mg, 1.17 mmol) and 4-(dimethylamino)aniline (500 mg, 3.67 mmol) according to a method similar to the method of Example 311-1).

¹H-NMR (CDCl₃) δ: 0.98 (6H, d, J=6.6 Hz), 1.38 (9H, s), 2.16-2.27 (1H, m), 2.40 (3H, s), 2.63 (3H, s), 2.77 (2H, d, J=7.4 Hz), 2.90 (6H, s), 3.42 (2H, s), 4.06 (2H, d, J=5.1 Hz), 4.20 (1H, brs), 6.58 (1H, brs), 6.66 (2H, d, J=8.1 Hz), 7.02 (2H, d, J=7.7 Hz), 7.18 (2H, d, J=8.1 Hz), 7.24 (2H, d, J=7.7 Hz).

2) 2-[5-(Aminomethyl)-6-isobutyl-2-methyl-4-(4-methylphenyl)pyridin-3-yl]-N-[4-(dimethylamino)phenyl]acetamide trihydrochloride (62 mg, yield 42%) was obtained as a violet powder from tert-butyl {[5-(2-{[4-(dimethylamino)phenyl]amino}-2-oxoethyl)-2-isobutyl-6-methyl-4-(4-methylphenyl)pyridin-3-yl]methyl}carbamate (100 mg, 0.268 mmol) according to a method similar to the method of Example 2-3).

¹H-NMR (DMSO-d₆)δ:0.99 (6H, d, J=6.4 Hz), 2.13-2.28 (1H, m), 2.38 (3H, s), 2.76 (3H, s), 3.01 (6H, s), 3.13 (2H, s), 3.77-3.86 (5H, m), 7.20 (2H, d, J=8.1 Hz), 7.35 (2H, d, J=8.1 Hz), 7.51 (2H, d, J=8.1 Hz), 8.30 (2H, d, J=8.1 Hz) 8.56 (3H, brs).

### Example 363

### ethyl 5-(aminomethyl)-4-(4-methylphenyl)-2,6-dineopentylnicotinate

1) A mixture of potassium 3-ethoxy-3-oxopropionate (7.6 g, 45 mmol), magnesium chloride (2.8 g, 30 mmol) and tetrahydrofuran (75 mL) was stirred at 50° C. for 4 hrs. The obtained suspension was allowed to cool to room temperature, and a reaction mixture obtained by stirring a mixture of tert-butylacetic acid (3.5 g, 30 mmol), N,N′-carbonyldiimidazole (5.8 g, 36 mmol) and tetrahydrofuran (50 mL) at room temperature for 1 hr was added dropwise to the suspension. The resulting mixture was stirred at room temperature for 3 days. The reaction mixture was partitioned between ethyl acetate and 0.5N hydrochloric acid. The organic layer was washed successively with saturated aqueous sodium hydrogen carbonate and saturated brine and dried over anhydrous magnesium sulfate. The solvent was evaporated under reduced pressure to give ethyl 5,5-dimethyl-3-oxohexanoate as a crude product (5.9 g). A mixture of the crude product (5.9 g), ammonium acetate (9.8 g, 127 mmol), acetic acid (1.45 mL, 25 mmol) and toluene (200 mL) was heated under reflux using a Dean-Stark trap for 17 hrs. The reaction mixture was allowed to cool to room temperature, washed with saturated brine and dried over anhydrous magnesium sulfate. The solvent was evaporated under reduced pressure and the residue was purified by silica gel column chromatography to give ethyl 3-amino-5,5-dimethylhex-2-enoate (2.5 g, yield 52%) as a white powder.

¹H-NMR (CDCl₃)δ: 1.00 (9H, s), 1.27 (3H, t, J=7.2 Hz), 1.98 (2H, s), 4.11 (2H, q, J=7.2 Hz), 4.45 (2H, brs), 8.05 (1H, s).

2) Ethyl 5-cyano-4-(4-methylphenyl)-2,6-dineopentyl-1,4-dihydropyridine-3-carboxylate (3.5 g, yield 65%) was obtained as a white powder from 5,5-dimethyl-3-oxohexanenitrile (2.4 g, 13 mmol), p-tolualdehyde (1.6 g, 13 mol) and ethyl 3-amino-5,5-dimethylhex-2-enoate (2.5 g, 13 mmol) according to a method similar to the method of Example 1-2).

¹H-NMR (CDCl₃)δ: 1.01 (9H, s), 1.03 (9H, s), 1.17 (3H, t, J=7.2 Hz), 2.06 (1H, d, J=13.7 Hz), 2.27 (1H, d, J=13.7 Hz), 2.31 (3H, s), 2.52 (1H, d, J=13.7 Hz), 3.34 (1H, d, J=13.7 Hz), 3.95-4.10 (2H, m), 4.63 (1H, s), 5.44 (1H, brs), 7.09 (2H, d, J=8.0 Hz), 7.17 (2H, d, J=8.0 Hz).

3) Ethyl 5-cyano-4-(4-methylphenyl)-2,6-dineopentylnicotinate (3.2 g, yield 96%) was obtained as a white powder from ethyl 5-cyano-4-(4-methylphenyl)-2,6-dineopentyl-1,4-dihydropyridine-3-carboxylate (3.4 g, 8.2 mmol) according to a method similar to the method of Example 23-3).

¹H-NMR (CDCl₃)δ: 0.91 (3H, t, J=7.2 Hz), 1.01 (9H, s), 1.08 (9H, s), 2.40 (3H, s), 2.87 (2H, s), 3.02 (2H, s), 3.99 (2H, q, J=7.2 Hz), 7.20-7.30 (4H, m).

4) Ethyl 5-(aminomethyl)-4-(4-methylphenyl)-2,6-dineopentylnicotinate (0.91 g, yield 90%) was obtained as a colorless oil from ethyl 5-cyano-4-(4-methylphenyl)-2,6-dineopentylnicotinate (1.0 g, 2.5 mmol) according to a method similar to the method of Example 1-4).

¹H-NMR (CDCl₃)δ: 0.89 (3H, t, J=7.2 Hz), 0.99 (9H, s), 1.04 (9H, s), 1.33 (2H, brs), 2.38 (3H, s), 2.78 (2H, s), 2.88 (2H, s), 3.72 (2H, s), 3.89 (2H, q, J=7.2 Hz), 7.12 (2H, d, J=8.0 Hz), 7.20 (2H, d, J=8.0 Hz).

### Example 364

### 3-{[5-(aminomethyl)-6-isobutyl-2-methyl-4-(4-methylphenyl)pyridin-3-yl]methoxy}propane-1-ol dihydrochloride

1) A mixture of [5-{[(tert-butoxycarbonyl)amino]methyl}-6-isobutyl-2-methyl-4-(4-methylphenyl)pyridin-3-yl]methyl methanesulfonate (1.91 g, 4.01 mmol), 1,3-propanediol (3.05 g, 40.1 mmol), sodium hydride (60% in oil, 1.60 g, 40.1 mmol) and tetrahydrofuran (5 mL) was stirred at 55° C. for 16 hrs. The reaction mixture was allowed to cool to room temperature and 1N hydrochloric acid was added to stop the reaction. The reaction mixture was diluted with ethyl acetate and washed with saturated brine. The organic layer was dried over magnesium sulfate and the solvent was evaporated under reduced pressure. The residue was purified by silica gel column chromatography to give tert-butyl {[5-[(3-hydroxypropoxy)methyl]-2-isobutyl-6-methyl-4-(4-methylphenyl)pyridin-3-yl]methyl}carbamate (840 mg, yield 46%) as a white powder.

¹H-NMR (CDCl₃) δ: 0.96 (6H, d, J=6.6 Hz), 1.38 (9H, s), 1.70-1.80 (2H, m), 2.16-2.27 (1H, m), 2.42 (3H, s), 2.63 (3H, s), 2.75 (2H, d, J=7.4 Hz), 3.40 (2H, t, J=5.8 Hz), 3.70 (2H, t, J=5.8 Hz), 4.06 (2H, d, J=4.7 Hz), 4.10 (2H, s), 4.20 (1H, brs), 7.03 (2H, d, J=7.9 Hz), 7.24 (2H, d, J=7.9 Hz).

2) 3-{[5-(Aminomethyl)-6-isobutyl-2-methyl-4-(4-methylphenyl)pyridin-3-yl]methoxy}propane-1-ol dihydrochloride (15 mg, yield 100%) was obtained as a white powder from tert-butyl {[5-[(3-hydroxypropoxy)methyl]-2-isobutyl-6-methyl-4-(4-methylphenyl)pyridin-3-yl]methyl}carbamate (18 mg, 0.0394 mmol) according to a method similar to the method of Example 2-3).

¹H-NMR (DMSO-d₆) δ:0.99 (6H, d, J=6.4 Hz), 1.70-2.3 (2H, m), 2.38 (3H, s), 2.75 (2H, s), 3.35-4.20 (6H, m), 4.06 (2H, d, J=4.5 Hz), 4.11 (2H, d, J=4.5 Hz), 7.00 (2H, d, J=8.1 Hz), 7.30 (2H, d, J=8.1 Hz), 7.51 (2H, d, J=8.1 Hz), 8.56 (3H, brs).

### Example 365

### 4-[({[5-(aminomethyl)-6-isobutyl-2-methyl-4-(4-methylphenyl)pyridin-3-yl]carbonyl}oxy)methyl]phthalic acid dihydrochloride

1) Dimethyl 4-[({[5-{[(tert-butoxycarbonyl)amino]methyl}-6-isobutyl-2-methyl-4-(4-methylphenyl)pyridin-3-yl]carbonyl}oxy)methyl]phthalate (1.68 g, yield 95%) was obtained as a colorless oil from 5-{[(tert-butoxycarbonyl)amino]methyl}-6-isobutyl-2-methyl-4-(4-methylphenyl)nicotinic acid (1.18 g, 2.86 mmol) and dimethyl 4-(bromomethyl)phthalate (820 mg, 2.86 mmol) according to a method similar to the method of Example 169-1).

¹H-NMR (CDCl₃) δ:0.97 (6H, d, J=6.6 Hz), 1.38 (9H, s), 2.17-2.26 (1H, m), 2.33 (3H, s), 2.54 (3H, s), 2.78 (2H, d, J=7.4 Hz), 3.92 (3H, s), 3.93 (3H, s), 4.11-4.15 (2H, m), 4.21 (1H, brs), 4.95 (2H, s), 7.00 (2H, d, J=8.1 Hz), 7.09 (2H, d, J=7.9 Hz), 7.16 (1H, dd, J=7.9, 1.7 Hz), 7.47 (1H, d, J=1.5 Hz), 7.62 (1H, d, J=7.7 Hz).

2) 4-[({[5-{[(tert-Butoxycarbonyl)amino]methyl}-6-isobutyl-2-methyl-4-(4-methylphenyl)pyridin-3-yl]carbonyl}oxy)methyl]phthalic acid (1.60 g, yield 99%) was obtained as a colorless oil from dimethyl 4-[({[5-{[(tert-butoxycarbonyl)amino]methyl}-6-isobutyl-2-methyl-4-(4-methylphenyl)pyridin-3-yl]carbonyl}oxy)methyl]phthalate (1.68 g, 2.72 mmol) according to a method similar to the method of Example 9-1).

¹H-NMR (CDCl₃) δ:1.00 (6H, d, J=6.6 Hz), 1.38 (9H, s), 2.16-2.27 (1H, m), 2.39 (3H, s), 2.67 (3H, brs), 3.10 (2H, d, J=7.0 Hz), 4.23 (2H, d, J=4.9 Hz), 4.51 (1H, brs), 5.01 (2H, S), 7.07 (2H, s), 7.21-7.24 (3H, m), 8.03 (1H, s), 8.13 (1H, d, J=7.9 Hz).

3) 4-[({[5-(Aminomethyl)-6-isobutyl-2-methyl-4-(4-methylphenyl)pyridin-3-yl]carbonyl}oxy)methyl]phthalic acid dihydrochloride (396 mg, yield 84%) was obtained as a white solid from 4-[({[5-{[(tert-butoxycarbonyl)amino]methyl}-6-isobutyl-2-methyl-4-(4-methylphenyl)pyridin-3-yl]carbonyl}oxy)methyl]phthalic acid (0.49 g, 0.830 mmol) according to a method similar to the method of Example 2-3).

¹H-NMR (DMSO-d₆) δ:0.96 (6H, d, J=6.6 Hz), 2.17-2.26 (1H, m), 2.33 (3H, s), 2.56 (3H, brs), 2.91 (2H, brs), 3.81 (2H, d, J=4.9 Hz), 5.05 (2H, s), 7.13 (2H, d, J=7.9 Hz), 7.17-7.21 (3H, m), 7.39 (1H, d, J=1.5 Hz), 7.59 (1H, d, J=7.9 Hz), 8.32 (3H, brs).

### Example 366

### 4-[({[5-(aminomethyl)-6-isobutyl-2-methyl-4-(4-methylphenyl)pyridin-3-yl]carbonyl}oxy)methyl]-2-fluorobenzoic acid dihydrochloride

1) 4-Bromo-3-fluorobenzyl 5-{[(tert-butoxycarbonyl)amino]methyl}-6-isobutyl-2-methyl-4-(4-methylphenyl)nicotinate (1.36 g, yield 78%) was obtained as a colorless oil from 5-{[(tert-butoxycarbonyl)amino]methyl}-6-isobutyl-2-methyl-4-(4-methylphenyl)nicotinic acid (1.20 g, 2.91 mmol) and (4-bromo-3-fluorophenyl)methanol (597 mg, 2.91 mmol) according to a method similar to the method of Example 247-1).

¹H-NMR (CDCl₃) δ:0.97 (6H, d, J=6.8 Hz), 1.39 (9H, s), 2.16-2.25 (1H, m), 2.36 (3H, s), 2.55 (3H, s), 2.78 (2H, d, J=7.2 Hz), 4.11-4.16 (2H, m), 4.21 (1H, brs), 4.86 (2H, s), 6.61-6.65 (1H, m), 7.00-7.06 (3H, m), 7.12-7.19 (3H, m).

2) 3-Fluoro-4-(methoxycarbonyl)benzyl 5-{[(tert-butoxycarbonyl)amino]methyl}-6-isobutyl-2-methyl-4-(4-methylphenyl)nicotinate (520 mg, yield 39%) was obtained as a yellow oil from 4-bromo-3-fluorobenzyl 5-{[(tert-butoxycarbonyl)amino]methyl}-6-isobutyl-2-methyl-4-(4-methylphenyl)nicotinate (1.36 g, 2.27 mmol) according to a method similar to the method of Example 231-2).

¹H-NMR (CDCl₃) δ:0.96 (6H, d, J=6.6 Hz), 1.38 (9H, s), 2.15-2.25 (1H, m), 2.33 (3H, s), 2.55 (3H, s), 2.78 (2H, d, J=7.4 Hz), 3.94 (3H, s), 4.09-4.15 (2H, m), 4.21 (1H, brs), 4.94 (2H, s), 6.81-6.85 (1H, m), 7.00 (2H, d, J=8.1 Hz), 7.10 (2H, d, J=7.9 Hz), 7.63-7.67 (2H, m).

3) 4-[({[5-{[(tert-Butoxycarbonyl)amino]methyl}-6-isobutyl-2-methyl-4-(4-methylphenyl)pyridin-3-yl]carbonyl}oxy)methyl]-2-fluorobenzoic acid (480 mg, yield 94%) was obtained as a colorless oil from 3-fluoro-4-(methoxycarbonyl)benzyl 5-{[(tert-butoxycarbonyl)amino]methyl}-6-isobutyl-2-methyl-4-(4-methylphenyl)nicotinate (520 mg, 0.899 mmol) according to a method similar to the method of Example 9-1).

¹H-NMR (CDCl₃) δ:0.97 (6H, d, J=6.6 Hz), 1.38 (9H, s), 2.16-2.26 (1H, m), 2.33 (3H, s), 2.56 (3H, s), 2.81 (2H, d, J=7.4 Hz), 4.09-4.16 (2H, m), 4.24 (1H, brs), 4.96 (2H, s), 6.88-6.92 (1H, m), 7.02 (2H, d, J=7.9 Hz), 7.11 (2H, d, J=7.9 Hz), 7.69-7.73 (2H, m).

4) 4-[({[5-(Aminomethyl)-6-isobutyl-2-methyl-4-(4-methylphenyl)pyridin-3-yl]carbonyl}oxy)methyl]-2-fluorobenzoic acid dihydrochloride (192 mg, yield 42%) was obtained as a white solid from 4-[({[5-{[(tert-butoxycarbonyl)amino]methyl}-6-isobutyl-2-methyl-4-(4-methylphenyl)pyridin-3-yl]carbonyl}oxy)methyl]-2-fluorobenzoic acid (480 mg, 0.850 mmol) according to a method similar to the method of Example 2-3).

¹H-NMR (DMSO-d₆) δ:0.96 (6H, d, J=6.8 Hz), 2.12-2.26 (1H, m), 2.30 (3H, s), 2.53 (3H, s), 2.86 (2H, d, J=7.0 Hz), 3.79 (2H, d, J=5.7 Hz), 5.05 (2H, s), 7.05-7.16 (5H, m), 7.59-7.64 (2H, m), 8.24 (3H, brs).

### Example 367

### N-[5-(aminomethyl)-6-isobutyl-2-methyl-4-(4-methylphenyl)pyridin-3-yl]-4-oxo-4,5,6,7-tetrahydro-1-benzofuran-3-carboxamide dihydrochloride

N-[5-(Aminomethyl)-6-isobutyl-2-methyl-4-(4-methylphenyl)pyridin-3-yl]-4-oxo-4,5,6,7-tetrahydro-1-benzofuran-3-carboxamide dihydrochloride (172 mg, yield 66%) was obtained as a white powder from tert-butyl {[5-amino-2-isobutyl-6-methyl-4-(4-methylphenyl)pyridin-3-yl]methyl}carbamate (192 mg, 0.5 mmol) and 4-oxo-4,5,6,7-tetrahydro-1-benzofuran-3-carbonyl chloride (150 mg, 0.75 mmol) according to a method similar to the method of Example 223.

¹H-NMR (DMSO-d₆) δ: 1.10 (6H, d, J=6.6 Hz), 2.00-2.09 (2H, m), 2.11-2.31 (1H, m), 2.31 (3H, s), 2.44 (2H, t, J=6.3 Hz), 2.59 (3H, s), 2.93 (2H, t, J=6.3 Hz), 3.06 (2H, s), 3.85 (2H, s), 7.24 (4H, s), 8.35 (1H, s), 8.36 (3H, brs), 11.42 (1H, brs).

### Example 368

### N-[5-(aminomethyl)-6-isobutyl-2-methyl-4-(4-methylphenyl)pyridin-3-yl]-2-phenyl-1,3-thiazole-4-carboxamide dihydrochloride

N-[5-(Aminomethyl)-6-isobutyl-2-methyl-4-(4-methylphenyl)pyridin-3-yl]-2-phenyl-1,3-thiazole-4-carboxamide dihydrochloride (155 mg, yield 57%) was obtained as a white powder from tert-butyl {[5-amino-2-isobutyl-6-methyl-4-(4-methylphenyl)pyridin-3-yl]methyl}carbamate (192 mg, 0.5 mmol) and 2-phenyl-1,3-thiazole-4-carbonyl chloride (167 mg, 0.75 mmol) according to a method similar to the method of Example 223.

¹H-NMR (DMSO-d₆) δ: 1.00 (6H, d, J=6.6 Hz), 2.20-2.29 (1H, m), 2.28 (3H, s), 2.61 (3H, s), 3.04 (2H, s), 3.85 (2H, s), 7.26 (4H, s), 7.53-7.55 (3H, m), 7.95-7.98 (2H, m), 8.35 (1H, s), 8.36 (3H, brs), 9.85 (1H, brs).

### Example 369

### N-[5-(aminomethyl)-6-isobutyl-2-methyl-4-(4-methylphenyl)pyridin-3-yl]pyrazine-2-carboxamide dihydrochloride

N-[5-(Aminomethyl)-6-isobutyl-2-methyl-4-(4-methylphenyl)pyridin-3-yl]pyrazine-2-carboxamide dihydrochloride (157 mg, yield 63%) was obtained as a white powder from tert-butyl {[5-amino-2-isobutyl-6-methyl-4-(4-methylphenyl)pyridin-3-yl]methyl}carbamate (192 mg, 0.5 mmol) and pyrazine-2-carbonyl chloride (107 mg, 0.75 mmol) according to a method similar to the method of Example 223.

¹H-NMR (DMSO-d₆) δ: 1.01 (6H, d, J=6.6 Hz), 2.18-2.28 (1H, m), 2.27 (3H, s), 2.63 (3H, s), 3.12 (2H, s), 3.85 (2H, s), 7.21 (2H, d, J=8.1 Hz), 7.26 (2H, d, J=8.1 Hz), 8.46 (3H, brs), 8.70 (1H, s), 8.88 (1H, s), 9.08 (1H, s), 10.48 (1H, brs).

### Example 370

### 4-[({[5-(aminomethyl)-2-methyl-4-(4-methylphenyl)-6-neopentylpyridin-3-yl]acetyl}oxy)methyl]benzoic acid dihydrochloride

1) 6N Hydrochloric acid (200 mL) was added to tert-butyl {[5-(cyanomethyl)-6-methyl-4-(4-methylphenyl)-2-neopentylpyridin-3-yl]methyl}carbamate (16 g, 37 mmol) and the mixture was stirred at 90° C. for 24 hrs. The reaction mixture was washed with a mixed solvent of tetrahydrofuran-toluene (1:2) and concentrated under reduced pressure. The residue was dissolved in water and alkalified by adding 4N aqueous sodium hydroxide solution. The obtained alkalified solution was washed with ethyl acetate and concentrated under reduced pressure. Tetrahydrofuran (100 mL) and water (50 mL) were added to the residue and the mixture was stirred vigorously. Di-tert-butyl dicarbonate (8.5 mL, 37 mmol) was added dropwise and the mixture was stirred at room temperature for 17 hrs. 1N Hydrochloric acid was added to the reaction mixture to acidify the aqueous layer and the mixture was extracted with ethyl acetate. The extracts were combined, washed with saturated brine and dried over anhydrous magnesium sulfate. The solvent was evaporated under reduced pressure and the residue was crystallized from hexane-ethyl acetate to give [5-{[(tert-butoxycarbonyl)amino]methyl}-2-methyl-4-(4-methylphenyl)-6-neopentylpyridin-3-yl]acetic acid (13 g, yield 80%) as a white powder.

¹H-NMR (CDCl₃)δ: 1.09 (9H, s), 1.39 (9H, s), 2.43 (3H, s), 2.82 (3H, d, J=20 Hz), 3.34 (2H, brs), 3.43 (2H, brs), 4.05-4.25 (2H, m), 4.35-4.50 (1H, m), 6.97 (2H, dd, J=7.5, 24 Hz), 7.26 (2H, dd, J=7.5, 29 Hz).

2) A mixture of [5-{[(tert-butoxycarbonyl)amino]methyl}-2-methyl-4-(4-methylphenyl)-6-neopentylpyridin-3-yl]acetic acid (0.50 g, 1.1 mmol), triethylamine (0.17 mL, 1.3 mmol) and tetrahydrofuran (20 mL) was ice-cooled and a solution of 2,4,6-trichlorobenzoyl chloride (0.31 g, 1.3 mmol) in tetrahydrofuran (2 mL) was added dropwise. The obtained mixture was stirred at room temperature for 14 hrs. The reaction mixture was filtered and the filtrate was concentrated under reduced pressure. The residue was dissolved in tetrahydrofuran (20 mL), and 2-oxo-2-phenylethyl 4-(hydroxymethyl)benzoate (0.37 g, 1.4 mmol) and 4-dimethylaminopyridine (0.17 g, 1.4 mmol) were added. The obtained solution was stirred at room temperature for 30 min. The reaction mixture was partitioned between ethyl acetate and water. The organic layer was washed successively with 0.1 M aqueous citric acid solution, saturated aqueous sodium hydrogen carbonate and saturated brine, and dried over anhydrous magnesium sulfate. The solvent was evaporated under reduced pressure and the residue was purified by silica gel column chromatography to give 2-oxo-2-phenylethyl 4-[({[5-{[(tert-butoxycarbonyl)amino]methyl}-2-methyl-4-(4-methylphenyl)-6-neopentylpyridin-3-yl]acetyl}oxy)methyl]benzoate (0.63 g, yield 80%) as a white powder.

¹H-NMR (CDCl₃)δ: 1.02 (9H, s), 1.37 (9H, s), 2.39 (3H, s), 2.49 (3H, s), 2.84 (2H, s), 3.43 (2H, s), 4.08 (2H, d, J=4.0 Hz), 4.15-4.25 (1H, m), 5.11 (2H, s), 5.59 (2H, s), 6.94 (2H, d, J=7.9 Hz), 7.17 (2H, d, J=7.9 Hz), 7.31 (2H, d, J=8.3 Hz), 7.45-7.55 (2H, m), 7.60-7.70 (1H, m), 7.95-8.00 (2H, m), 8.11 (2H, d, J=8.3 Hz).

3) 2-Oxo-2-phenylethyl 4-[({[5-{[(tert-butoxycarbonyl)amino]methyl}-2-methyl-4-(4-methylphenyl)-6-neopentylpyridin-3-yl]acetyl}oxy)methyl]benzoate (0.61 g, 0.88 mmol) was dissolved in ethyl acetate (2 mL) and water (2 mL), acetic acid (5 mL) and zinc powder (0.42 g, 6.4 mmol) were successively added to the obtained solution. The resulting mixture was stirred at 55° C. for 24 hrs. The reaction mixture was filtered and the filtrate was concentrated under reduced pressure. The obtained residue was partitioned between ethyl acetate and water. The organic layer was washed with saturated brine and dried over anhydrous magnesium sulfate. The solvent was evaporated under reduced pressure, and the residue was purified by silica gel column chromatography and further recrystallized from hexane-ethyl acetate to give 4-[({(5-{[(tert-butoxycarbonyl)amino]methyl}-2-methyl-4-(4-methylphenyl)-6-neopentylpyridin-3-yl]acetyl}oxy)methyl]benzoic acid (0.29 g, yield 48%) as a white powder.

¹H-NMR (CDCl₃)δ: 1.02 (9H, s), 1.36 (9H, s), 2.38 (3H, s), 2.47 (3H, s), 2.88 (2H, s), 3.43 (2H, s), 4.10 (2H, d, J=5.1 Hz), 4.15-4.25 (1H, m), 5.11 (2H, s), 6.94 (2H, d, J=7.7 Hz), 7.17 (2H, d, J=7.7 Hz), 7.30 (2H, d, J=8.1 Hz), 8.07 (2H, d, J=8.1 Hz).

4) 4-[({[5-(Aminomethyl)-2-methyl-4-(4-methylphenyl)-6-neopentylpyridin-3-yl]acetyl}oxy)methyl]benzoic acid dihydrochloride (0.22 g, yield 92.4%) was obtained as a pale-yellow powder from 4-[({[5-{[(tert-butoxycarbonyl)amino]methyl}-2-methyl-4-(4-methylphenyl)-6-neopentylpyridin-3-yl]acetyl}oxy)methyl]benzoic acid (0.25 g, 0.44 mmol) according to a method similar to the method of Example 276-3).

¹H-NMR (DMSO-d₆)δ: 1.01 (9H, s), 2.37 (3H, s), 2.73 (3H, brs), 3.00-3.30 (2H, m), 3.57 (2H, brs), 3.82 (2H, brs), 5.11 (2H, s), 7.09 (2H, d, J=7.9 Hz), 7.28 (2H, d, J=7.9 Hz), 7.34 (2H, d, J=8.2 Hz), 7.94 (2H, d, J=8.2 Hz), 8.19 (3H, brs).

### Example 371

### 2-[({[5-(aminomethyl)-6-isobutyl-2-methyl-4-(4-methylphenyl)pyridin-3-yl]carbonyl}oxy)methyl]furan-3-carboxylic acid dihydrochloride

1) [3-(Methoxycarbonyl)-2-furyl]methyl 5-{[(tert-butoxycarbonyl)amino]methyl}-6-isobutyl-2-methyl-4-(4-methylphenyl)nicotinate (320 mg, yield 47%) was obtained as a colorless oil from 5-{[(tert-butoxycarbonyl)amino]methyl}-6-isobutyl-2-methyl-4-(4-methylphenyl)nicotinic acid (0.50 g, 1.22 mmol) and methyl 2-(bromomethyl)furan-3-carboxylate (266 mg, 1.22 mmol) according to a method similar to the method of Example 169-1).

¹H-NMR (CDCl₃) δ:0.96 (6H, d, J=6.8 Hz), 1.38 (9H, s), 2.15-2.26 (1H, m), 2.37 (3H, s), 2.55 (3H, s), 2.77 (2H, d, J=7.4 Hz), 3.82 (3H, s), 4.09-4.13 (2H, m), 4.19 (1H, brs), 5.27 (2H, s), 6.68 (1H, d, J=1.9 Hz), 7.00 (2H, d, J=8.1 Hz), 7.11 (2H, d, J=7.9 Hz), 7.31 (1H, d, J=1.9 Hz).

2) 2-[({[5-{[(tert-Butoxycarbonyl)amino]methyl}-6-isobutyl-2-methyl-4-(4-methylphenyl)pyridin-3-yl]carbonyl}oxy)methyl]furan-3-carboxylic acid (310 mg, yield 99%) was obtained as a colorless oil from [3-(methoxycarbonyl)-2-furyl]methyl 5-{[(tert-butoxycarbonyl)amino]methyl}-6-isobutyl-2-methyl-4-(4-methylphenyl)nicotinate (320 mg, 0.581 mmol) according to a method similar to the method of Example 9-1).

¹H-NMR (CDCl₃) δ:0.96 (6H, d, J=6.6 Hz), 1.38 (9H, s), 2.13-2.22 (1H, m), 2.37 (3H, s), 2.55 (3H, s), 2.80 (2H, d, J=7.4 Hz), 4.09-4.16 (2H, m), 4.23 (1H, brs), 5.27 (2H, s), 6.72 (1H, d, J=1.9 Hz), 7.02 (2H, d, J=7.9 Hz), 7.13 (2H, d, J=7.4 Hz), 7.34 (1H, d, J=1.9 Hz).

3) 2-[({[5-(Aminomethyl)-6-isobutyl-2-methyl-4-(4-methylphenyl)pyridin-3-yl]carbonyl}oxy)methyl]furan-3-carboxylic acid dihydrochloride (241 mg, yield 81%) was obtained as a pale-yellow solid from 2-[({[5-{[(tert-butoxycarbonyl)amino]methyl}-6-isobutyl-2-methyl-4-(4-methylphenyl)pyridin-3-yl]carbonyl}oxy)methyl]furan-3-carboxylic acid (310 mg, 0.577 mmol) according to a method similar to the method of Example 2-3).

¹H-NMR (DMSO-d₆)δ:0.96 (6H, d, J=6.6 Hz), 2.16-2.25 (1H, m), 2.35 (3H, s), 2.53 (3H, brs), 2.90 (2H, brs), 3.80 (2H, d, J=5.1 Hz), 5.26 (2H, s), 6.71 (1H, d, J=1.9 Hz), 7.12 (2H, d, J=7.9 Hz), 7.19 (2H, d, J=7.9 Hz), 7.72 (1H, d, J=1.9 Hz), 8.32 (3H, brs).

### Example 372

### 4-[({[5-(aminomethyl)-6-isobutyl-2-methyl-4-(4-methylphenyl)pyridin-3-yl]carbonyl}oxy)methyl]-3-nitrobenzoic acid dihydrochloride

1) 4-(Methoxycarbonyl)-2-nitrobenzyl 5-{[(tert-butoxycarbonyl)amino]methyl}-6-isobutyl-2-methyl-4-(4-methylphenyl)nicotinate (1.91 g, yield 63%) was obtained as a colorless oil from 5-{[(tert-butoxycarbonyl)amino]methyl}-6-isobutyl-2-methyl-4-(4-methylphenyl)nicotinic acid (1.91 g, 4.63 mmol) and methyl 4-(hydroxymethyl)-3-nitrobenzoate (978 mg, 4.63 mmol) according to a method similar to the method of Example 247-1).

¹H-NMR (CDCl₃) δ:0.98 (6H, d, J=6.6 Hz), 1.39 (9H, s), 2.18-2.28 (1H, m), 2.34 (3H, s), 2.57 (3H, s), 2.79 (2H, d, J=7.4 Hz), 3.99 (3H, s), 4.10-4.17 (2H, m), 4.23 (1H, brs), 5.41 (2H, s), 7.03-7.09 (3H, m), 7.13 (2H, d, J=7.9 Hz), 8.08 (1H, dd, J=8.1, 1.5 Hz), 8.68 (1H, d, J=1.5 Hz).

2) 4-[({[5-{[(tert-Butoxycarbonyl)amino]methyl}-6-isobutyl-2-methyl-4-(4-methylphenyl)pyridin-3-yl]carbonyl}oxy)methyl]-3-nitrobenzoic acid (300 mg, yield 93%) was obtained as a colorless oil from 4-(methoxycarbonyl)-2-nitrobenzyl 5-{[(tert-butoxycarbonyl)amino]methyl}-6-isobutyl-2-methyl-4-(4-methylphenyl)nicotinate (0.33 g, 0.545 mmol) according to a method similar to the method of Example 9-1).

¹H-NMR (CDCl₃) δ:0.99 (6H, d, J=6.6 Hz), 1.39 (9H, s), 2.18-2.34 (4H, m), 2.59 (3H, s), 2.83 (2H, d, J=6.8 Hz), 4.10-4.18 (2H, m), 4.26 (1H, brs), 5.42 (2H, s), 7.02-7.20 (5H, m), 8.12-8.16 (1H, m), 8.73 (1H, s).

3) 4-[({[5-(Aminomethyl)-6-isobutyl-2-methyl-4-(4-methylphenyl)pyridin-3-yl]carbonyl}oxy)methyl]-3-nitrobenzoic acid dihydrochloride (247 mg, yield 86%) was obtained as a white solid from 4-[({[5-{[(tert-butoxycarbonyl)amino]methyl}-6-isobutyl-2-methyl-4-(4-methylphenyl)pyridin-3-yl]carbonyl}oxy)methyl]-3-nitrobenzoic acid (300 mg, 0.507 mmol) according to a method similar to the method of Example 2-3).

¹H-NMR (DMSO-d₆)δ:0.97 (6H, d, J=6.8 Hz), 2.16-2.25 (1H, m), 2.29 (3H, s), 2.60 (3H, brs), 2.94-3.00 (2H, m), 3.81 (2H, d. J=5.5 Hz), 5.42 (2H, s), 7.17 (4H, s), 7.24 (1H, d, J=8.1 Hz), 8.13 (1H, dd, J=8.1, 1.7 Hz), 8.39 (3H, brs), 8.48 (1H, d, J=1.7 Hz).

### Example 373

### methyl 3-{[5-(aminomethyl)-2-methyl-4-(4-methylphenyl)-6-neopentylpyridin-3-yl]methoxy}-1-methyl-1H-pyrazole-4-carboxylate dihydrochloride

1) Ethyl 3-{[5-{[(tert-butoxycarbonyl)amino]methyl}-2-methyl-4-(4-methylphenyl)-6-neopentylpyridin-3-yl]methoxy}-1-methyl-1H-pyrazole-4-carboxylate (2.34 g, yield 81%) was obtained as a colorless oil from tert-butyl {[5-(hydroxymethyl)-6-methyl-4-(4-methylphenyl)-2-neopentylpyridin-3-yl]methyl}carbamate (2.09 g, 5.07 mmol) and ethyl 3-hydroxy-1-methyl-1H-pyrazole-4-carboxylate (863 mg, 5.07 mmol) according to a method similar to the method of Example 183-1).

¹H-NMR (CDCl₃) δ:1.03 (9H, s), 1.26-1.28 (3H, m), 1.37 (9H, s), 2.36 (3H, s), 2.66 (3H, s), 2.86 (2H, s), 3.68 (3H, s), 4.13 (1H, brs), 4.23 (2H, q, J=7.1 Hz), 4.90 (2H, s), 7.11 (2H, d, J=8.3 Hz), 7.16 (2H, d, J=8.1 Hz), 7.62 (1H, s).

2) 3-{[5-{[(tert-Butoxycarbonyl)amino]methyl}-2-methyl-4-(4-methylphenyl)-6-neopentylpyridin-3-yl]methoxy}-1-methyl-1H-pyrazole-4-carboxylic acid (2.22 g, yield 99%) was obtained as a colorless oil from ethyl 3-{[5-{[(tert-butoxycarbonyl)amino]methyl}-2-methyl-4-(4-methylphenyl)-6-neopentylpyridin-3-yl]methoxy}-1-methyl-1H-pyrazole-4-carboxylate (2.34 g, 4.14 mmol) according to a method similar to the method of Example 9-1).

¹H-NMR (CDCl₃) δ:1.04 (9H, s), 1.37 (9H, s), 2.35 (3H, s), 2.66 (3H, s), 2.88 (2H, s), 3.70 (3H, s), 4.09-4.18 (2H, m), 4.24 (1H, brs), 4.95 (2H, s), 7.08 (2H, d, J=7.5 Hz), 7.18 (2H, d, J=7.7 Hz), 7.68 (1H, s).

3) Methyl 3-{[5-{[(tert-butoxycarbonyl)amino]methyl}-2-methyl-4-(4-methylphenyl)-6-neopentylpyridin-3-yl]methoxy}-1-methyl-1H-pyrazole-4-carboxylate (480 mg, yield 91%) was obtained as a colorless oil from 3-{[5-{[(tert-butoxycarbonyl)amino]methyl}-2-methyl-4-(4-methylphenyl)-6-neopentylpyridin-3-yl]methoxy}-1-methyl-1H-pyrazole-4-carboxylic acid (0.51 g, 0.950 mmol) according to a method similar to the method of Example 305-3).

¹H-NMR (CDCl₃) δ:1.03 (9H, s), 1.37 (9H, s), 2.36 (3H, s), 2.66 (3H, s), 2.86 (2H, s), 3.68 (3H, s), 3.76 (3H, s), 4.09-4.17 (2H, m), 4.19 (1H, brs), 4.90 (2H, s), 7.10 (2H, d, J=8.1 Hz), 7.16 (2H, d, J=8.1 Hz), 7.62 (1H, s).

4) Methyl 3-{[5-(aminomethyl)-2-methyl-4-(4-methylphenyl)-6-neopentylpyridin-3-yl]methoxy}-1-methyl-1H-pyrazole-4-carboxylate dihydrochloride (349 mg, yield 76%) was obtained as a white solid from methyl 3-{[5-{[(tert-butoxycarbonyl)amino]methyl}-2-methyl-4-(4-methylphenyl)-6-neopentylpyridin-3-yl]methoxy}-1-methyl-1H-pyrazole-4-carboxylate (480 mg, 0.872 mmol) according to a method similar to the method of Example 2-3).

¹H-NMR (DMSO-d₆)δ:1.05 (9H, s), 2.38 (3H, s), 2.91 (3H, brs), 3.28 (2H, brs), 3.65 (3H, s), 3.66 (3H, s), 3.89 (2H, brs), 4.90 (2H, s), 7.27 (2H, d, J=7.9 Hz), 7.33 (2H, d, J=8.1 Hz), 8.09 (1H, s), 8.32 (3H, brs).

### Example 374

### 3-{[5-(aminomethyl)-2-methyl-4-(4-methylphenyl)-6-neopentylpyridin-3-yl]methoxy}-1-methyl-1H-pyrazole-4-carboxylic acid dihydrochloride

3-{[5-(Aminomethyl)-2-methyl-4-(4-methylphenyl)-6-neopentylpyridin-3-yl]methoxy}-1-methyl-1H-pyrazole-4-carboxylic acid dihydrochloride (210 mg, yield 76%) was obtained as a white solid from 3-{[5-{[(tert-butoxycarbonyl)amino]methyl}-2-methyl-4-(4-methylphenyl)-6-neopentylpyridin-3-yl]methoxy}-1-methyl-1H-pyrazole-4-carboxylic acid (0.29 g, 0.540 mmol) according to a method similar to the method of Example 2-3).

¹H-NMR (DMSO-d₆)δ:1.04 (9H, s), 2.38 (3H, s), 2.87 (3H, brs), 3.23 (2H, brs), 3.64 (3H, s), 3.89 (2H, brs), 4.86 (2H, s), 7.27 (2H, d, J=7.9 Hz), 7.33 (2H, d, J=7.9 Hz), 8.00 (1H, s), 8.26 (3H, brs).

### Example 375

### 3-{[5-(aminomethyl)-2-methyl-4-(4-methylphenyl)-6-neopentylpyridin-3-yl]methoxy}-1-methyl-1H-pyrazole-4-carboxamide dihydrochloride

1) tert-Butyl {[5-({[4-(aminocarbonyl)-1-methyl-1H-pyrazol-3-yl]oxy}methyl)-6-methyl-4-(4-methylphenyl)-2-neopentylpyridin-3-yl]methyl}carbamate (110 mg, yield 18%) was obtained as a colorless oil from 3-{[5-{[(tert-butoxycarbonyl)amino]methyl}-2-methyl-4-(4-methylphenyl)-6-neopentylpyridin-3-yl]methoxy}-1-methyl-1H-pyrazole-4-carboxylic acid (0.60 g, 1.12 mmol) according to a method similar to the method of Example 3-1).

¹H-NMR (CDCl₃) δ:1.04 (9H, s), 1.37 (9H, s), 2.37 (3H, s), 2.64 (3H, s), 2.87 (2H, s), 3.69 (3H, s), 4.11-4.16 (2H, m), 4.97 (2H, s), 5.24 (1H, brs), 6.43 (1H, brs), 7.01 (2H, d, J=7.7 Hz), 7.20 (2H, d, J=8.3 Hz), 7.69 (1H, s).

2) 3-{[5-(Aminomethyl)-2-methyl-4-(4-methylphenyl)-6-neopentylpyridin-3-yl]methoxy}-1-methyl-1H-pyrazole-4-carboxamide dihydrochloride (70.3 mg, yield 67%) was obtained as a white solid from tert-butyl [[5-({[4 (aminocarbonyl)-1-methyl-1H-pyrazol-3-yl]oxy}methyl)-6-methyl-4-(4-methylphenyl)-2-neopentylpyridin-3-yl]methyl}carbamate (110 mg, 0.205 mmol) according to a method similar to the method of Example 2-3).

¹H-NMR (DMSO-d₆)δ:1.04 (9H, s), 2.38 (3H, s), 2.91 (3H, brs), 3.25 (2H, brs), 3.63 (3H, s), 3.88 (2H, brs), 4.92 (2H, s), 6.35 (1H, brs), 7.09 (1H, brs), 7.27 (2H, d, J=7.0 Hz), 7.34 (2H, d, J=7.5 Hz), 7.91 (1H, s), 8.29 (3H, brs).

### Example 376

### {2-[({[5-(aminomethyl)-6-isobutyl-2-methyl-4-(4-methylphenyl)pyridin-3-yl]carbonyl}oxy)methyl]phenyl}acetic acid dihydrochloride

1) 2-(2-Ethoxy-2-oxoethyl)benzyl 5-{[(tert-butoxycarbonyl)amino]methyl}-6-isobutyl-2-methyl-4-(4-methylphenyl)nicotinate (980 mg, yield 70%) was obtained as a colorless oil from 5-{[(tert-butoxycarbonyl)amino]methyl}-6-isobutyl-2-methyl-4-(4-methylphenyl)nicotinic acid (1.00 g, 2.42 mmol) and ethyl [2-(bromomethyl)phenyl]acetate (624 mg, 2.42 mmol) according to a method similar to the method of Example 169-1).

¹H-NMR (CDCl₃)δ:0.96 (6H, d, J=6.8 Hz), 1.20 (3H, t, J=7.2 Hz), 1.38 (9H, s), 2.15-2.26 (1H, m), 2.35 (3H, s), 2.51 (3H, s), 2.77 (2H, d, J=7.4 Hz), 3.51 (2H, s), 4.02-4.09 (2H, m), 4.09-4.13 (2H, m), 4.19 (1H, brs), 5.02 (2H, s), 6.99 (2H, d, J=8.3 Hz), 7.06-7.08 (3H, m), 7.16-7.21 (2H, m), 7.26-7.31 (1H, m).

2) {2-[({[5-{[(tert-Butoxycarbonyl)amino]methyl}-6-isobutyl-2-methyl-4-(4-methylphenyl)pyridin-3-yl]carbonyl}oxy)methyl]phenyl}acetic acid (600 mg, yield 62%) was obtained as a colorless oil from 2-(2-ethoxy-2-oxoethyl)benzyl 5-{[(tert-butoxycarbonyl)amino]methyl}-6-isobutyl-2-methyl-4-(4-methylphenyl)nicotinate (980 mg, 1.71 mmol) according to a method similar to the method of Example 9-1).

¹H-NMR (CDCl₃) δ:0.93 (6H, d, J=6.8 Hz), 1.37 (9H, s), 2.10-2.21 (1H, m), 2.34 (3H, s), 2.49 (3H, s), 2.76 (2H, d, J=7.2 Hz), 3.53 (2H, s), 4.05-4.13 (2H, m), 4.29 (1H, brs), 5.01 (2H, s), 6.98 (2H, d, J=8.3 Hz), 7.02-7.11 (3H, m), 7.18-7.32 (3H, m).

3) {2-[({[5-(Aminomethyl)-6-isobutyl-2-methyl-4-(4-methylphenyl)pyridin-3-yl]carbonyl}oxy)methyl]phenyl}acetic acid dihydrochloride (125 mg, yield 62%) was obtained as a white solid from {2-[({[5-{[(tert-butoxycarbonyl)amino]methyl}-6-isobutyl-2-methyl-4-(4-methylphenyl)pyridin-3-yl]carbonyl}oxy)methyl]phenyl}acetic acid (210 mg, 0.374 mmol) according to a method similar to the method of Example 2-3).

¹H-NMR (DMSO-d₆)δ:0.96 (6H, d, J=6.6 Hz), 2.16-2.28 (1H, m), 2.36 (3H, s), 2.88 (2H, brs), 3.47 (2H, s), 3.81 (2H, d, J=5.1 Hz), 4.99 (2H, s), 6.98 (1H, d, J=7.5 Hz), 7.13-7.32 (7H, m), 8.27 (3H, brs)

### Example 377

### 2-(2-amino-2-oxoethyl)benzyl 5-(aminomethyl)-6-isobutyl-2-methyl-4-(4-methylphenyl)nicotinate dihydrochloride

1) 2-(2-Amino-2-oxoethyl)benzyl 5-{[(tert-butoxycarbonyl)amino]methyl}-6-isobutyl-2-methyl-4-(4-methylphenyl)nicotinate (323 mg, yield 83%) was obtained as a colorless oil from {2-[({[5-{[(tert-butoxycarbonyl)amino]methyl}-6-isobutyl-2-methyl-4-(4-methylphenyl)pyridin-3-yl]carbonyl}oxy)methyl]phenyl}acetic acid (0.39 g, 0.695 mmol) according to a method similar to the method of Example 3-1).

¹H-NMR (CDCl₃)δ:0.96 (6H, d, J=6.6 Hz), 1.38 (9H, s), 2.13-2.26 (1H, m), 2.35 (3H, s), 2.50 (3H, s), 2.76 (2H, d, J=7.4 Hz), 3.47 (2H, s), 4.06-4.13 (2H, m), 4.24 (1H, brs), 5.01 (2H, s), 6.99 (2H, d, J=8.1 Hz), 7.06-7.10 (3H, m), 7.19-7.35 (3H, m).

2) 2-(2-Amino-2-oxoethyl)benzyl 5-(aminomethyl)-6-isobutyl-2-methyl-4-(4-methylphenyl)nicotinate dihydrochloride (209 mg, yield 68%) was obtained as a white solid from 2-(2-amino-2-oxoethyl)benzyl 5-{[(tert-butoxycarbonyl)amino]methyl}-6-isobutyl-2-methyl-4-(4-methylphenyl)nicotinate (323 mg, 0.577 mmol) according to a method similar to the method of Example 2-3).

¹H-NMR (DMSO-d₆)δ:0.96 (6H, d, J=6.6 Hz), 2.14-2.25 (1H, m), 2.36 (3H, s), 2.55 (3H, s), 2.93 (2H, brs), 3.32 (2H, s), 3.82 (2H, d, J=5.1 Hz), 5.08 (2H, s), 6.94 (2H, d, J=7.4 Hz), 7.14-7.30 (7H, m), 7.51 (1H, brs), 8.35 (3H, brs).

### Example 378

### methyl 3-{[5-(aminomethyl)-6-isobutyl-2-methyl-4-(4-methylphenyl)pyridin-3-yl]methoxy}thiophene-2-carboxylate dihydrochloride

1) Methyl 3-{[5-{[(tert-butoxycarbonyl)amino]methyl}-6-isobutyl-2-methyl-4-(4-methylphenyl)pyridin-3-yl]methoxy}thiophene-2-carboxylate (460 mg, yield 68%) was obtained as a colorless oil from tert-butyl {[5-(hydroxymethyl)-2-isobutyl-6-methyl-4-(4-methylphenyl)pyridin-3-yl]methyl}carbamate (0.50 g, 1.25 mmol) and methyl 3-hydroxythiophene-2-carboxylate (0.20 g, 1.25 mmol) according to a method similar to the method of Example 214-1).

¹H-NMR (CDCl₃) δ:0.98 (6H, d, J=6.8 Hz), 1.39 (9H, s), 2.18-2.27 (1H, m), 2.38 (3H, s), 2.72 (3H, s), 2.77 (2H, d, J=7.4 Hz), 3.80 (3H, s), 4.06-4.11 (2H, m), 4.20 (1H, brs), 4.79 (2H, s), 6.50 (1H, d, J=5.5 Hz), 7.06 (2H, d, J=8.1 Hz), 7.18 (2H, d, J=7.9 Hz), 7.29 (1H, d, J=5.5 Hz).

2) Methyl 3-{[5-(aminomethyl)-6-isobutyl-2-methyl-4-(4-methylphenyl)pyridin-3-yl]methoxy}thiophene-2-carboxylate dihydrochloride (126 mg, yield 84%) was obtained as a white solid from methyl 3-{[5-{[(tert-butoxycarbonyl)amino]methyl}-6-isobutyl-2-methyl-4-(4-methylphenyl)pyridin-3-yl]methoxy}thiophene-2-carboxylate (158 mg, 0.293 mmol) according to a method similar to the method of Example 2-3).

¹H-NMR (DMSO-d₆)δ:0.99 (6H, d, J=6.4 Hz), 2.15-2.28 (1H, m), 2.37 (3H, s), 2.88 (3H, s), 3.11 (2H, brs), 3.71 (3H, s), 3.82 (2H, s), 4.87 (2H, s), 6.86 (1H, d, J=5.7 Hz), 7.21-7.34 (4H, m), 7.77 (1H, d, J=5.5 Hz), 8.36 (3H, brs).

### Example 379

### methyl 4-{[5-(aminomethyl)-6-isobutyl-2-methyl-4-(4-methylphenyl)pyridin-3-yl]methoxy}-2-methyl-1,3-thiazole-5-carboxylate dihydrochloride

1) Ethyl 4-{[5-{[(tert-butoxycarbonyl)amino]methyl}-6-isobutyl-2-methyl-4-(4-methylphenyl)pyridin-3-yl]methoxy}-2-methyl-1,3-thiazole-5-carboxylate (910 mg, yield 96%) was obtained as a colorless oil from tert-butyl {[5-(hydroxymethyl)-2-isobutyl-6-methyl-4-(4-methylphenyl)pyridin-3-yl]methyl}carbamate (0.66 g, 1.66 mmol) and ethyl 4-hydroxy-2-methyl-1,3-thiazole-5-carboxylate (0.31 g, 1.66 mmol) according to a method similar to the method of Example 214-1).

¹H-NMR (CDCl₃) δ:0.98 (6H, d, J=6.6 Hz), 1.28 (3H, t, J=7.2 Hz), 1.39 (9H, s), 2.17-2.26 (1H, m), 2.37 (3H, s), 2.53 (3H, s), 2.77 (2H, d, J=7.2 Hz), 4.08 (2H, d, J=4.5 Hz), 4.25 (2H, q, J=7.0 Hz), 5.13 (2H, s), 7.09 (2H, d, J=8.1 Hz), 7.16 (2H, d, J=7.9 Hz).

2) 4-{[5-{[(tert-Butoxycarbonyl)amino]methyl}-6-isobutyl-2-methyl-4-(4-methylphenyl)pyridin-3-yl]methoxy}-2-methyl-1,3-thiazole-5-carboxylic acid (750 mg, yield 87%) was obtained as a colorless oil from ethyl 4-{[5-{[(tert-butoxycarbonyl)amino]methyl}-6-isobutyl-2-methyl-4-(4-methylphenyl)pyridin-3-yl]methoxy}-2-methyl-1,3-thiazole-5-carboxylate (910 mg, 1.60 mmol) according to a method similar to the method of Example 9-1).

¹H-NMR (CDCl₃) δ:1.01 (6H, d, J=6.6 Hz), 1.38 (9H, s), 2.18-2.30 (1H, m), 2.38 (3H, s), 2.57 (3H, s), 2.81 (3H, brs), 2.95 (2H, d, J=7.0 Hz), 4.09-4.15 (2H, m), 4.31 (1H, brs), 5.22 (2H, s), 7.05 (2H, d, J=7.9 Hz), 7.22 (2H, d, J=7.9 Hz).

3) Methyl 4-{[5-{[(tert-butoxycarbonyl)amino]methyl}-6-isobutyl-2-methyl-4-(4-methylphenyl)pyridin-3-yl]methoxy}-2-methyl-1,3-thiazole-5-carboxylate (420 mg, yield 77%) was obtained as a pale-yellow solid from 4-{[5-{[(tert-butoxycarbonyl)amino]methyl}-6-isobutyl-2-methyl-4-(4-methylphenyl)pyridin-3-yl]methoxy}-2-methyl-1,3-thiazole-5-carboxylic acid (530 mg, 0.982 mmol) according to a method similar to the method of Example 305-3).

¹H-NMR (CDCl₃) δ:0.98 (6H, d, J=6.6 Hz), 1.39 (9H, s), 2.15-2.27 (1H, m), 2.37 (3H, s), 2.54 (3H, s), 2.68 (3H, s), 2.77 (2H, d, J=7.4 Hz), 3.79 (3H, s), 4.08 (2H, d, J=4.9 Hz), 4.21 (1H, brs), 5.14 (2H, s), 7.09 (2H, d, J=8.1 Hz), 7.16 (2H, d, J=7.9 Hz).

4) Methyl 4-{[5-(aminomethyl)-6-isobutyl-2-methyl-4-(4-methylphenyl)pyridin-3-yl]methoxy}-2-methyl-1,3-thiazole-5-carboxylate dihydrochloride (342 mg, yield 85%) was obtained as a pale-yellow solid from methyl 4-{[5-{[(tert-butoxycarbonyl)amino]methyl}-6-isobutyl-2-methyl-4-(4-methylphenyl)pyridin-3-yl]methoxy}-2-methyl-1,3-thiazole-5-carboxylate (420 mg, 0.759 mmol) according to a method similar to the method of Example 2-3).

¹H-NMR (DMSO-d₆)δ:0.99 (6H, d, J=6.6 Hz), 2.13-2.28 (1H, m), 2.38 (3H, s), 2.55 (3H, s), 2.93 (3H, brs), 3.13 (2H, brs), 3.70 (3H, s), 3.80 (2H, brs), 5.17 (2H, s), 7.20-7.26 (2H, m), 7.31 (2H, d, J=7.4 Hz), 8.38 (3H, brs).

### Example 380

### 4-{[5-(aminomethyl)-6-isobutyl-2-methyl-4-(4-methylphenyl)pyridin-3-yl]methoxy}-2-methyl-1,3-thiazole-5-carboxylic acid dihydrochloride

4-{[5-(Aminomethyl)-6-isobutyl-2-methyl-4-(4-methylphenyl)pyridin-3-yl]methoxy}-2-methyl-1,3-thiazole-5-carboxylic acid dihydrochloride (145 mg, yield 69%) was obtained as a white solid from 4-{[5-{[(tert-butoxycarbonyl)amino]methyl}-6-isobutyl-2-methyl-4-(4-methylphenyl)pyridin-3-yl]methoxy}-2-methyl-1,3-thiazole-5-carboxylic acid (220 mg, 0.408 mmol) according to a method similar to the method of Example 2 3).

¹H-NMR (DMSO-d₆)δ:0.99 (6H, d, J=6.6 Hz), 2.15-2.28 (1H, m), 2.38 (3H, s), 2.53 (3H, s), 2.90 (3H, brs), 3.10 (2H, brs), 3.75-3.85 (2H, m), 5.11 (2H, s), 7.25 (2H, d, J=6.4 Hz), 7.32 (2H, d, J=7.7 Hz), 8.15-8.42 (3H, m).

### Example 381

### 3-{[5-(aminomethyl)-6-isobutyl-2-methyl-4-(4-methylphenyl)pyridin-3-yl]methoxy}-1-(carboxymethyl)-1H-pyrazole-4-carboxylic acid dihydrochloride

1) Ethyl 1-acetyl-3-{[5-{[(tert-butoxycarbonyl)amino]methyl}-6-isobutyl-2-methyl-4-(4-methylphenyl)pyridin-3-yl]methoxy}-1H-pyrazole-4-carboxylate (1.12 g, yield 77%) was obtained as a white solid from tert-butyl {[5-(hydroxymethyl)-2-isobutyl-6-methyl-4-(4-methylphenyl)pyridin-3-yl]methyl}carbamate (1.00 g, 2.51 mmol) and ethyl 1-acetyl-3-hydroxy-1H-pyrazole-4-carboxylate (597 mg, 3.01 mmol) according to a method similar to the method of Example 214-1).

¹H-NMR (CDCl₃) δ:0.98 (6H, d, J=6.8 Hz), 1.31 (3H, t, J=7.2 Hz), 1.39 (9H, s), 2.14-2.27 (1H, m), 2.36 (3H, s), 2.51 (3H, S), 2.67 (3H, s), 2.78 (2H, d, J=7.4 Hz), 4.09 (2H, d, J=5.1 Hz), 4.20 (1H, brs), 4.28 (2H, q, J=7.1 Hz), 5.01 (2H, s), 7.09 (2H, d, J=8.1 Hz), 7.17 (2H, d, J=7.9 Hz), 8.49 (1H, s).

2) To a solution of ethyl 1-acetyl-3-{[5-{[(tert-butoxycarbonyl)amino]methyl}-6-isobutyl-2-methyl-4-(4-methylphenyl)pyridin-3-yl]methoxy}-1H-pyrazole-4-carboxylate (0.86 g, 1.49 mmol) in tetrahydrofuran (10 mL)—methanol (5 mL) was added saturated aqueous sodium hydrogen carbonate (10 mL) and the mixture was stirred at room temperature for 30 min. The reaction mixture was diluted with ethyl acetate, washed with saturated brine and dried over anhydrous magnesium sulfate. The solvent was evaporated under reduced pressure and the obtained residue was purified by silica gel column chromatography to give ethyl 3-{[5-{[(tert-butoxycarbonyl)amino]methyl}-6-isobutyl-2-methyl-4-(4-methylphenyl)pyridin-3-yl]methoxy}-1H-pyrazole-4-carboxylate (798 mg, yield 99%) as a colorless oil.

¹H-NMR (CDCl₃) δ:0.91 (3H, d, J=6.6 Hz), 0.97 (3H, d, J=6.6 Hz), 1.24-1.29 (3H, m), 1.40-1.46 (9H, m), 2.19-2.28 (1H, m), 2.36 (3H, brs), 2.65-2.78 (5H, m), 3.87-4.04 (2H, m), 4.08-4.35 (5H, m), 4.87 (1H, brs), 6.91-7.01 (2H, m), 7.07-7.15 (2H, m), 7.84 (1H, s).

3) To a solution of ethyl 3-{[5-{[(tert-butoxycarbonyl)amino]methyl}-6-isobutyl-2-methyl-4-(4-methylphenyl)pyridin-3-yl]methoxy}-1H-pyrazole-4-carboxylate (1.09 g, 2.03 mmol) in N,N-dimethylformamide (20 mL) was added sodium hydride (60% in oil, 98 mg, 2.44 mmol) and the mixture was stirred at room temperature for 30 min. tert-Butyl bromoacetate (0.36 mL, 2.44 mmol) was added to the reaction mixture and the mixture was stirred with heating at 60° C. for 30 min. The reaction mixture was diluted with ethyl acetate, washed with saturated brine and dried over anhydrous magnesium sulfate. The solvent was evaporated under reduced pressure and the obtained residue was purified by silica gel column chromatography to give ethyl 3-{[5-{[(tert-butoxycarbonyl)amino]methyl}-6-isobutyl-2-methyl-4-(4-methylphenyl)pyridin-3-yl]methoxy}-1-(2-tert-butoxy-2-oxoethyl)-1H-pyrazole-4-carboxylate (960 mg, yield 72%) as a colorless oil.

¹H-NMR (CDCl₃) δ:0.98 (6H, d, J=6.6 Hz), 1.28 (3H, t, J=7.1 Hz), 1.39 (9H, s), 1.44 (9H, s), 2.14-2.25 (1H, m), 2.36 (3H, s), 2.66 (3H, s), 2.76 (2H, d, J=7.4 Hz), 4.08 (2H, d, J=4.9 Hz), 4.17-4.27 (3H, m), 4.52 (2H, s), 4.91 (2H, s), 7.09 (2H, d, J=8.1 Hz), 7.16 (2H, d, J=8.1 Hz), 7.73 (1H, s).

4) To a mixed solution of ethyl 3-{[5-{[(tert-butoxycarbonyl)amino]methyl}-6-isobutyl-2-methyl-4-(4-methylphenyl)pyridin-3-yl]methoxy}-1-(2-tert-butoxy-2-oxoethyl)-1H-pyrazole-4-carboxylate (960 mg, 1.48 mmol) in tetrahydrofuran (15 mL) methanol (10 mL) was added 1N aqueous sodium hydroxide solution (10 mL) and the mixture was heated under reflux for 1 hr. The reaction mixture was allowed to cool to room temperature and acidified with 0.5N hydrochloric acid. The mixture was extracted with ethyl acetate, and the extract was washed with saturated brine and dried over anhydrous magnesium sulfate. The solvent was evaporated under reduced pressure to give 3-{[5-{[(tert-butoxycarbonyl)amino]methyl}-6-isobutyl-2-methyl-4-(4-methylphenyl)pyridin-3-yl]methoxy}-1-(carboxymethyl)-1H-pyrazole-4-carboxylic acid (838 mg, yield 99%) as an oil. 3-{[5-(Aminomethyl)-6-isobutyl-2-methyl-4-(4-methylphenyl)pyridin-3-yl]methoxy}-1-(carboxymethyl)-1H-pyrazole-4-carboxylic acid dihydrochloride (58.2 mg, yield 59%) was obtained as a white solid from the obtained oil (107 mg, 0.189 mmol) according to a method similar to the method of Example 2-3).

¹H-NMR (DMSO-d₆)δ:0.99 (6H, d, J=6.6 Hz), 2.14-2.28 (1H, m), 2.38 (3H, s), 2.82 (3H, brs), 3.04 (2H, brs), 3.76-3.86 (2H, m), 4.77 (2H, s), 4.86 (2H, s), 7.26 (2H, d, J=8.1 Hz), 7.32 (2H, d, J=7.9 Hz), 8.04 (1H, s), 8.27 (3H, brs).

### Example 382

### methyl 3-{[5-(aminomethyl)-6-isobutyl-2-methyl-4-(4-methylphenyl)pyridin-3-yl]methoxy}-1-(2-methoxy-2-oxoethyl)-1H-pyrazole-4-carboxylate dihydrochloride

1) Methyl 3-{[5-{[(tert-butoxycarbonyl)amino]methyl}-6-isobutyl-2-methyl-4-(4-methylphenyl)pyridin-3-yl]methoxy}-1-(2-methoxy-2-oxoethyl)-1H-pyrazole-4-carboxylate (560 mg, 0.636 mmol) was obtained as a colorless oil from 3-{[5-{[(tert-butoxycarbonyl)amino]methyl}-6-isobutyl-2-methyl-4-(4-methylphenyl)pyridin-3-yl]methoxy}-1-(carboxymethyl)-1H-pyrazole-4-carboxylic acid (870 mg, 1.48 mmol) according to a method similar to the method of Example 305-3).

¹H-NMR (CDCl₃) δ:0.98 (6H, d, J=6.6 Hz), 1.39 (9H, s), 2.15-2.25 (1H, m), 2.36 (3H, s), 2.66 (3H, s), 2.76 (2H, d, J=7.2 Hz), 3.76 (3H, s), 3.77 (3H, s), 4.08 (2H, d, J=4.7 Hz), 4.22 (1H, brs), 4.65 (2H, s), 4.91 (2H, s), 7.08 (2H, d, J=8.1 Hz), 7.16 (2H, d, J=7.9 Hz), 7.74 (1H, s).

2) Methyl 3-{[5-(aminomethyl)-6-isobutyl-2-methyl-4-(4-methylphenyl)pyridin-3-yl]methoxy}-1-(2-methoxy-2-oxoethyl)-1H-pyrazole-4-carboxylate dihydrochloride (59.8 mg, yield 63%) was obtained as a white solid from methyl 3-{[5-{[(tert-butoxycarbonyl)amino]methyl}-6-isobutyl-2-methyl-4-(4-methylphenyl)pyridin-3-yl]methoxy}-1-(2-methoxy-2-oxoethyl)-1H-pyrazole-4-carboxylate (98.7 mg, 0.166 mmol) according to a method similar to the method of Example 2-3).

¹H-NMR (DMSO-d₆)δ:0.98 (6H, d, J=6.6 Hz), 2.15-2.28 (1H, m), 2.37 (3H, s), 2.74 (3H, brs), 2.94 (2H, brs), 3.67 (3H, s), 3.68 (3H, s), 4.86 (2H, s), 4.91 (2H, s), 7.23 (2H, d, J=8.1 Hz), 7.29 (2H, d, J=8.1 Hz), 8.09-8.19 (4H, m).

### Example 383

### [3-{[5-(aminomethyl)-6-isobutyl-2-methyl-4-(4-methylphenyl)pyridin-3-yl]methoxy}-4-(methoxycarbonyl)-1H-pyrazol-1-yl]acetic acid dihydrochloride

1) To a solution of methyl 3-{[5-{[(tert-butoxycarbonyl)amino]methyl}-6-isobutyl-2-methyl-4-(4-methylphenyl)pyridin-3-yl]methoxy}-1-(2-methoxy-2-oxoethyl)-1H-pyrazole-4-carboxylate (0.46 g, 0.775 mmol) in tetrahydrofuran was added 1N aqueous sodium hydroxide solution (1 mL) and the mixture was stirred at room temperature for 30 min. The reaction mixture was acidified with 0.5N hydrochloric acid and extracted with ethyl acetate. The extract was washed with saturated brine and dried over anhydrous magnesium sulfate. The solvent was evaporated under reduced pressure to give [3-{[5-{[(tert-butoxycarbonyl)amino]methyl}-6-isobutyl-2-methyl-4-(4-methylphenyl)pyridin-3-yl]methoxy}-4-(methoxycarbonyl)-1H-pyrazol-1-yl]acetic acid (450 mg, yield 99%) as a colorless oil.

¹H-NMR (CDCl₃) δ:1.03 (6H, d, J-6.6 Hz), 1.38 (9H, s), 2.21-2.34 (1H, m), 2.43 (3H, s), 3.02-3.26 (5H, m), 3.76 (3H, s), 4.13-4.19 (2H, m), 4.62 (2H, s), 4.99-5.11 (2H, m), 7.12 (2H, d, J=7.0 Hz), 7.30 (2H, d, J=7.5 Hz), 7.68-7.75 (1H, m).

2) [3-{[5-(Aminomethyl)-6-isobutyl-2-methyl-4-(4-methylphenyl)pyridin-3-yl]methoxy}-4-(methoxycarbonyl)-1H-pyrazol-1-yl]acetic acid dihydrochloride (42.4 mg, yield 44%) was obtained as a white solid from [3-{[5-{[(tert-butoxycarbonyl)amino]methyl}-6-isobutyl-2-methyl-4-(4-methylphenyl)pyridin-3-yl]methoxy}-4-(methoxycarbonyl)-1H-pyrazol-1-yl]acetic acid (100 mg, 0.172 mmol) according to a method similar to the method of Example 2-3).

¹H-NMR (DMSO-d₆)δ:0.99 (6H, d, J=6.6 Hz), 2.14-2.28 (1H, m), 2.38 (3H, s), 2.85 (3H, brs), 3.07 (2H, brs), 3.68 (3H, s), 3.75-3.85 (2H, m), 4.78 (2H, s), 4.90 (2H, s), 7.25 (2H, d, J=7.9 Hz), 7.31 (2H, d, J=7.9 Hz), 8.12 (1H, s), 8.31 (3H, brs).

### Example 384

### methyl 3-{[5-(aminomethyl)-6-isobutyl-2-methyl-4-(4-methylphenyl)pyridin-3-yl]methoxy}-1-(2-amino-2-oxoethyl)-1H-pyrazole-4-carboxylate dihydrochloride

1) Methyl 1-(2-amino-2-oxoethyl)-3-{[5-{[(tert-butoxycarbonyl)amino]methyl}-6-isobutyl-2-methyl-4-(4-methylphenyl)pyridin-3-yl]methoxy}-1H-pyrazole-4-carboxylate (150 mg, yield 37%) was obtained as a white solid from [3-{[5-{[(tert-butoxycarbonyl)amino]methyl}-6-isobutyl-2-methyl-4-(4-methylphenyl)pyridin-3-yl]methoxy}-4-(methoxycarbonyl)-1H-pyrazol-1-yl]acetic acid (400 mg, 0.689 mmol) according to a method similar to the method of Example 3-1).

¹H-NMR (CDCl₃) δ:0.99 (6H, d, J=6.6 Hz), 1.39 (9H, s), 2.18-2.29 (1H, m), 2.36 (3H, s), 2.68 (3H, s), 2.77 (2H, d, J=7.4 Hz), 3.78 (3H, s), 4.08 (2H, d, J=5.1 Hz), 4.22 (1H, brs), 4.54 (2H, s), 4.94 (2H, s), 7.09 (2H, d, J=8.1 Hz), 7.16 (2H, d, J=7.9 Hz), 7.74 (1H, s).

Methyl 3-{[5-(aminomethyl)-6-isobutyl-2-methyl-4-(4-methylphenyl)pyridin-3-yl]methoxy}-1-(2-amino-2-oxoethyl)-1H-pyrazole-4-carboxylate dihydrochloride (141 mg, yield 98%) was obtained as a white solid from methyl 1-(2-amino-2-oxoethyl)-3-{[5-{[(tert-butoxycarbonyl)amino]methyl}-6-isobutyl-2-methyl-4-(4-methylphenyl)pyridin-3-yl]methoxy}-1H-pyrazole-4-carboxylate (150 mg, 0.259 mmol) according to a method similar to the method of Example 2-3).

¹H-NMR (DMSO-d₆)δ:0.99 (6H, d, J=6.6 Hz), 2.14-2.27 (1H, m), 2.39 (3H, s), 2.86 (3H, brs), 3.09 (2H, brs), 3.67 (3H, s), 3.81 (2H, d, J=4.7 Hz), 4.58 (2H, s), 4.89 (2H, s), 7.26 (2H, d, J=8.1 Hz), 7.32 (2H, d, J=8.1 Hz), 7.58 (1H, s), 8.33 (3H, brs).

### Example 385

### N-[5-(aminomethyl)-6-isobutyl-2-methyl-4-(4-methylphenyl)pyridin-3-yl]terephthalamide dihydrochloride

1) tert-Butyl {[5-{[4-(aminocarbonyl)benzoyl]amino}-2-isobutyl-6-methyl-4-(4-methylphenyl)pyridin-3-yl]methyl}carbamate (248 mg, yield 98%) was obtained as a white powder from 4-({[5-{[(tert-butoxycarbonyl)amino]methyl}-6-isobutyl-2-methyl-4-(4-methylphenyl)pyridin-3-yl]amino}carbonyl)benzoic acid (260 mg, 0.48 mmol) according to a method similar to the method of Example 3-1).

¹H-NMR (CDCl₃) δ:0.99 (6H, d, J=6.6 Hz), 1.38 (9H, s), 2.15-2.29 (1H, m), 2.34 (3H, s), 2.50 (3H, s), 2.79 (2H, brs), 4.13 (2H, brs), 4.37 (1H, brs), 5.84 (1H, brs), 6.33 (1H, brs), 7.05 (2H, d, J=8.1 Hz), 7.19 (2H, d, J=8.1 Hz), 7.37 (1H, brs), 7.47 (2H, d, J=8.1 Hz), 7.73 (2H, d, J=8.1 Hz).

2) N-[5-(Aminomethyl)-6-isobutyl-2-methyl-4-(4-methylphenyl)pyridin-3-yl]terephthalamide dihydrochloride (233 mg, yield 99%) was obtained as a white powder from tert-butyl {[5-{[4-(aminocarbonyl)benzoyl]amino}-2-isobutyl-6-methyl-4-(4-methylphenyl)pyridin-3-yl]methyl}carbamate (248 mg, 0.47 mmol) according to a method similar to the method of Example 2-3).

¹H-NMR (DMSO-d₆) δ:1.01 (6H, d, J=6.3 Hz), 2.19-2.31 (1H, m), 2.31 (3H, s), 2.61 (3H, s), 3.05 (2H, brs), 3.85 (2H, brs), 7.25 (4H, s), 7.51 (1H, brs), 7.68 (2H, d, J=8.1 Hz), 7.89 (2H, d, J=8.1 Hz), 8.09 (1H, brs), 8.37 (3H, brs), 10.16 (1H, brs).

### Example 386

### ethyl 1-({[5-(aminomethyl)-6-isobutyl-2-methyl-4-(4-methylphenyl)pyridin-3-yl]amino}carbonyl)piperidine-4-carboxylate dihydrochloride

1) Ethyl 1-({[5-{[(tert-butoxycarbonyl)amino]methyl}-6-isobutyl-2-methyl-4-(4-methylphenyl)pyridin-3-yl]amino}carbonyl)piperidine-4-carboxylate was obtained as an oil from 5-{[(tert-butoxycarbonyl)amino]methyl}-6-isobutyl-2-methyl-4-(4-methylphenyl)nicotinic acid (412 mg, 1.0 mmol) and ethyl isonipecotate (314 mg, 2.0 mmol) according to a method similar to the method of Example 95-1).

EIMS(M+1):567

2) Ethyl 1-({[5-(aminomethyl)-6-isobutyl-2-methyl-4-(4-methylphenyl)pyridin-3-yl]amino}carbonyl)piperidine-4-carboxylate dihydrochloride (324 mg, yield 69%) was obtained as a white powder from the oil obtained in the aforementioned 1), according to a method similar to the method of Example 2-3).

¹H-NMR (DMSO-d₆)δ:0.98 (6H, d, J=6.3 Hz), 1.20 (3H, t, J=6.9 Hz), 1.54-1.59 (2H, m), 2.10-2.28 (1H, m), 2.34 (3H, s), 2.36-2.46 (1H, m), 2.62-2.76 (4H, m), 3.09 (2H, brs), 3.74-3.82 (4H, m), 4.07 (2H, q, J=6.9 Hz), 7.19 (2H, d, J=7.5 Hz), 7.26 (2H, d, J=7.5 Hz), 8.17 (1H, brs), 8.45 (3H, brs).

### Example 387

### ethyl 2-[({[5-(aminomethyl)-6-isobutyl-2-methyl-4-(4-methylphenyl)pyridin-3-yl]amino)carbonyl}amino]-1,3-oxazole-4-carboxylate dihydrochloride

1) Ethyl 2-[({[5-{[(tert-butoxycarbonyl)amino]methyl}-6-isobutyl-2-methyl-4-(4-methylphenyl)pyridin-3-yl]amino}carbonyl)amino]-1,3-oxazole-4-carboxylate was obtained as an oil from 5-{[(tert-butoxycarbonyl)amino]methyl}-6-isobutyl-2-methyl-4-(4-methylphenyl)nicotinic acid (412 mg, 1.0 mmol) and ethyl 2-amino-1,3-oxazole-4-carboxylate (312 mg, 2.0 mmol) according to a method similar to the method of Example 95-1).

EIMS(M+1):566

2) Ethyl 2-[({[5-(aminomethyl)-6-isobutyl-2-methyl-4-(4-methylphenyl)pyridin-3-yl]amino}carbonyl)amino]-1,3-oxazole-4-carboxylate dihydrochloride (224 mg, yield 48%) was obtained as a white powder from the oil obtained in the aforementioned 1), according to a method similar to the method of Example 2-3).

¹H-NMR (DMSO-d₆) δ:0.99 (6H, d, J=6.6 Hz), 1.29 (3H, t, J=7.2 Hz), 2.13-2.26 (1H, m), 2.29 (3H, s), 2.63 (3H, s), 3.06 (2H, brs), 3.82 (2H, s), 4.27 (2H, q, J=7.2 Hz), 7.15-7.29 (4H, m), 8.44 (3H, brs), 8.45 (1H, s), 9.32 (1H, brs), 11.14 (1H, brs).

### Example 388

### ethyl 2-[({[5-(aminomethyl)-6-isobutyl-2-methyl-4-(4-methylphenyl)pyridin-3-yl]amino)carbonyl)amino]-1,3-thiazole-4-carboxylate dihydrochloride

1) Ethyl 2-[({[5-{[(tert-butoxycarbonyl)amino]methyl}-6-isobutyl-2-methyl-4-(4-methylphenyl)pyridin-3-yl]amino}carbonyl)amino]-1,3-thiazole-4-carboxylate was obtained as an oil from 5-{[(tert-butoxycarbonyl)amino]methyl}-6-isobutyl-2-methyl-4-(4-methylphenyl)nicotinic acid (412 mg, 1.0 mmol) and ethyl 2-amino-1,3-thiazole-4-carboxylate (344 mg, 2.0 mmol) according to a method similar to the method of Example 95-1).

EIMS(M+1):582

2) Ethyl 2-[({[5-(aminomethyl)-6-isobutyl-2-methyl-4-(4-methylphenyl)pyridin-3-yl]amino}carbonyl)amino]-1,3-thiazole-4-carboxylate dihydrochloride (282 mg, yield 51%) was obtained as a white powder from the oil obtained in the aforementioned 1), according to a method similar to the method of Example 2-3).

1H-NMR (DMSO-d₆) δ:1.00 (6H, d, J=6.6 Hz), 1.27 (3H, t, J=7.2 Hz), 2.11-2.30 (1H, m), 2.35 (3H, s), 2.62 (3H, s), 3.06 (2H, brs), 3.81 (2H, s), 4.24 (2H, q, J=7.2 Hz), 7.21 (2H, d, J=7.8 Hz), 7.30 (2H, d, J=7.8 Hz), 7.91 (1H, s), 8.42 (3H, S), 8.76 (1H, brs), 11.21 (1H, brs).

### Example 389

### N-[5-(aminomethyl)-6-isobutyl-2-methyl-4-(4-methylphenyl)pyridin-3-yl]-4-phenylpiperidine-1-carboxamide dihydrochloride

1) tert-Butyl [(2-isobutyl-6-methyl-4-(4-methylphenyl)-5-{[(4-phenylpiperidin-1-yl)carbonyl]amino}pyridin-3-yl)methyl]carbamate was obtained as an oil from 5-{[(tert-butoxycarbonyl)amino]methyl}-6-isobutyl-2-methyl-4-(4-methylphenyl)nicotinic acid (412 mg, 1.0 mmol) and 4-phenylpiperidine (322 mg, 2.0 mmol) according to a method similar to the method of Example 95-1).

EIMS(M+1):571

2) N-[5-(Aminomethyl)-6-isobutyl-2-methyl-4-(4-methylphenyl)pyridin-3-yl]-4-phenylpiperidine-1-carboxamide dihydrochloride (240 mg, yield 44%) was obtained as a white powder from the oil obtained in the aforementioned 1), according to a method similar to the method of Example 2-3).

¹H-NMR (DMSO-d₆)δ:0.99 (6H, d, J=6.6 Hz), 1.54-1.58 (2H, m), 2.14-2.26 (1H, m), 2.26 (3H, s), 2.50 (3H, s), 2.58-2.75 (5H, m), 3.12 (2H, brs), 3.82 (2H, brs), 3.95-3.99 (2H, m), 7.11-7.37 (9H, m), 8.19 (1H, brs), 8.44 (1H, brs).

### Example 390

### methyl 1-({[5-(aminomethyl)-6-isobutyl-2-methyl-4-(4-methylphenyl)pyridin-3-yl]amino}carbonyl)pyrrolidine-2-carboxylate dihydrochloride

1) Methyl 1-({[5-{[(tert-butoxycarbonyl)amino]methyl}-6-isobutyl-2-methyl-4-(4-methylphenyl)pyridin-3-yl]amino}carbonyl)pyrrolidine-2-carboxylate was obtained as an oil from 5-{[(tert-butoxycarbonyl)amino]methyl}-6-isobutyl-2-methyl-4-(4-methylphenyl)nicotinic acid (412 mg, 1.0 mmol) and DL-proline methyl ester (286 mg, 2.0 mmol) according to a method similar to the method of Example 95-1).

EIMS(M+1):539

2) Methyl 1-({[5-(aminomethyl)-6-isobutyl-2-methyl-4-(4-methylphenyl)pyridin-3-yl]amino}carbonyl)pyrrolidine-2-carboxylate dihydrochloride (400 mg, yield 78%) was obtained as a white powder from the oil obtained in the aforementioned 1), according to a method similar to the method of Example 2-3).

¹H-NMR (DMSO-d₆)δ:0.98 (6H, d, J=6.6 Hz), 1.80 (3H, brs), 2.04-2.09 (1H, m), 2.11-2.23 (1H, m), 2.39 (3H, s), 2.63 (2H, s), 3.07 (4H, brs), 3.59 (3H, s), 3.86 (2H, brs), 4.11-4.17 (1H, m), 4.11-4.17 (1H, m), 7.21 (2H, d, J=7.8 Hz), 7.32 (2H, d, J=7.8 Hz), 7.93 (1H, brs), 8.39 (3H, brs).

### Example 391

### ethyl 1-({[5-(aminomethyl)-6-isobutyl-2-methyl-4-(4-methylphenyl)pyridin-3-yl]amino}carbonyl)piperidine-3-carboxylate dihydrochloride

1) Ethyl 1-({[5-{[(tert-butoxycarbonyl)amino]methyl}-6-isobutyl-2-methyl-4-(4-methylphenyl)pyridin-3-yl]amino}carbonyl)piperidine-3-carboxylate was obtained as an oil from 5-{[(tert-butoxycarbonyl)amino]methyl}-6-isobutyl-2-methyl-4-(4-methylphenyl)nicotinic acid (412 mg, 1.0 mmol) and ethyl 3-piperidinecarboxylate (314 mg, 2.0 mmol) according to a method similar to the method of Example 95-1).

EIMS(M+1):567

2) Ethyl 1-({[5-(aminomethyl)-6-isobutyl-2-methyl-4-(4-methylphenyl)pyridin-3-yl]amino}carbonyl)piperidine-3-carboxylate dihydrochloride (256 mg, yield 48%) was obtained as a white powder from the oil obtained in the aforementioned 1), according to a method similar to the method of Example 2-3).

¹H-NMR (DMSO-d₆)δ:0.98 (6H, d, J=6.6 Hz), 1.19 (3H, t, J=6.9 Hz), 1.39-1.46 (2H, m), 1.78 (2H, brs), 2.16-2.23 (1H, m), 2.37 (3H, s), 2.57 (2H, s), 3.03 (2H, s), 3.66-3.72 (1H, m), 3.82 (2H, brs), 4.05 (2H, q, J=6.9 Hz), 7.17 (2H, d, J-8.1 Hz), 7.30 (2H, d, J=8.1 Hz), 8.11 (1H, brs), 8.29 (3H, brs).

### Example 392

### 2-[5-(aminomethyl)-6-isobutyl-2-methyl-4-(4-methylphenyl)pyridin-3-yl]tetrahydroimidazo[1,5-a]pyridine-1,3(2H,5H)-dione dihydrochloride

1) tert-Butyl {[5-(1,3-dioxohexahydroimidazo[1,5-a]pyridin-2(3H)-yl)-2-isobutyl-6-methyl-4-(4-methylphenyl)pyridin-3-yl]methyl}carbamate was obtained as an oil from 5-{[(tert-butoxycarbonyl)amino]methyl}-6-isobutyl-2-methyl-4-(4-methylphenyl)nicotinic acid (412 mg, 1.0 mmol) and ethyl 2-piperidinecarboxylate (314 mg, 2.0 mmol) according to a method similar to the method of Example 95-1).

EIMS(M+1):553

2) 2-[5-(Aminomethyl)-6-isobutyl-2-methyl-4-(4-methylphenyl)pyridin-3-yl]tetrahydroimidazo[1,5-a]pyridine-1,3(2H,5H)-dione dihydrochloride (282 mg, yield 57%) was obtained as a white powder from the oil obtained in the aforementioned 1), according to a method similar to the method of Example 2-3).

¹H-NMR (DMSO-d₆)δ:0.99 (6H, d, J=6.6 Hz), 1.20-1.35 (1H, m), 1.36-1.50 (1H, m), 1.59-1.65 (1H, m), 1.79 (1H, brs), 1.99 (1H, brs), 2.22-2.31 (1H, m), 2.32 (6H, s), 2.35 (3H, s), 2.70-2.74 (1H, m), 2.82 (2H, d, J=6.9 Hz), 3.72-3.78 (4H, m), 7.05-7.09 (2H, m), 7.10-7.27 (2H, m), 8.13 (3H, brs).

### Example 393

### 2-[5-(aminomethyl)-6-isobutyl-2-methyl-4-(4-methylphenyl)pyridin-3-yl]-10,10a-dihydroimidazo[1,5-b]isoquinoline-1,3(2H,5H)-dione dihydrochloride

1) tert-Butyl {[5-(1,3-dioxo-1,5,10,10a-tetrahydroimidazo[1,5-b]isoquinolin-2(3H)-yl)-2-isobutyl-6-methyl-4-(4-methylphenyl)pyridin-3-yl]methyl}carbamate was obtained as an oil from 5-{[(tert-butoxycarbonyl)amino]methyl}-6-isobutyl-2-methyl-4-(4-methylphenyl)nicotinic acid (412 mg, 1.0 mmol) and ethyl 1,2,3,4 tetrahydroisoquinoline-3-carboxylate (410 mg, 2.0 mmol) according to a method similar to the method of Example 95-1).

EIMS(M+1):569

2) 2-[5-(Aminomethyl)-6-isobutyl-2-methyl-4-(4-methylphenyl)pyridin-3-yl]-10,10a-dihydroimidazo[1,5-b]isoquinoline-1,3(2H,5H)-dione dihydrochloride (368 mg, yield 68%) was obtained as a white powder from the oil obtained in the aforementioned 1), according to a method similar to the method of Example 2-3).

¹H-NMR (DMSO-d₆)δ:0.99 (6H, d, J=6.6 Hz), 2.18-2.34 (1H, m), 2.30 (3H, s), 2.34 (3H, s), 2.34 (2H, d, J=7.2 Hz), 2.95 (1H, dd, J=9.9, 17.1 Hz), 3.16 (1H, dd, J=4.8, 15.6 Hz), 3.78 (2H, m), 4.06 (1H, dd, J=9.9, 4.8 Hz), 4.08 (1H, d, J=17.1 Hz), 4.79 (1H, d, J=15.6 Hz), 7.07-7.31 (8H, m), 8.18 (3H, brs).

### Example 394

### methyl 2-({[5-(aminomethyl)-6-isobutyl-2-methyl-4-(4-methylphenyl)pyridin-3-yl]amino}carbonyl)benzoate dihydrochloride

Methyl 2-({[5-(aminomethyl)-6-isobutyl-2-methyl-4-(4-methylphenyl)pyridin-3-yl]amino}carbonyl)benzoate dihydrochloride (230 mg, yield 89%) was obtained as a white powder from tert-butyl {[5-amino-2-isobutyl-6-methyl-4-(4-methylphenyl)pyridin-3-yl]methyl}carbamate (192 mg, 0.5 mmol) and methyl 2-(chlorocarbonyl)benzoate (149 mg, 0.75 mmol) according to a method similar to the method of Example 223.

¹H-NMR (DMSO-d₆) δ:1.00 (6H, d, J=6.6 Hz), 2.18-2.27 (1H, m), 2.40 (3H, s), 2.66 (3H, s), 2.95 (2H, brs), 3.77 (3H, s), 3.79 (2H, brs), 6.58 (1H, d, J=7.5 Hz), 7.23 (2H, d, J=7.8 Hz), 7.34 (2H, d, J=7.8 Hz), 7.49 (1H, t, J=7.5 Hz), 7.53 (1H, t, J=7.5 Hz), 7.70 (1H, d, J=7.5 Hz), 8.25 (3H, brs), 10.03 (1H, brs).

### Example 395

### 2-({[5-(aminomethyl)-6-isobutyl-2-methyl-4-(4-methylphenyl)pyridin-3-yl]amino}carbonyl)benzoic acid dihydrochloride

1) 2-({[5-{[(tert-Butoxycarbonyl)amino]methyl}-6-isobutyl-2-methyl-4-(4-methylphenyl)pyridin-3-yl]amino}carbonyl)benzoic acid (247 mg, yield 98%) was obtained as a white powder from methyl 2-({[5-{[(tert-butoxycarbonyl)amino]methyl}-6-isobutyl-2-methyl-4-(4-methylphenyl)pyridin-3-yl]amino}carbonyl)benzoate (260 mg, 0.48 mmol) according to a method similar to the method of Example 36-1).

¹H-NMR (CDCl₃) δ:0.92 (6H, d, J=6.6 Hz), 1.38 (9H, s), 2.04-2.18 (1H, m), 2.41 (3H, s), 2.55 (3H, s), 2.82 (2H, brs), 4.09 (3H, brs), 6.17 (1H, brs), 6.91 (1H, brs), 7.09 (2H, brs), 7.25-7.27 (3H, m), 7.37 (1H, brs), 7.88 (1H, brs).

2) 2-({[5-(Aminomethyl)-6-isobutyl-2-methyl-4-(4-methylphenyl)pyridin-3-yl]amino}carbonyl)benzoic acid dihydrochloride (220 mg, yield 94%) was obtained as a white powder from 2-({[5-{[(tert-butoxycarbonyl)amino]methyl}-6-isobutyl-2-methyl-4-(4-methylphenyl)pyridin-3-yl]amino}carbonyl)benzoic acid (247 mg, 0.47 mmol) according to a method similar to the method of Example 2-3).

¹H-NMR (DMSO-d₆) δ:1.00 (6H, d, J=6.6 Hz), 2.18-2.26 (1H, m), 2.43 (3H, s), 2.74 (3H, s), 3.05 (2H, brs), 3.86 (2H, brs), 6.38 (1H, d, J=6.9 Hz), 7.25 (2H, d, J=8.1 Hz), 7.37 (2H, d, J=8.1 Hz), 7.41 (1H, t, J=6.9 Hz), 7.49 (1H, t, J=6.9 Hz), 7.76 (1H, d, J-=6.9 Hz), 8.35 (3H, brs), 10.02 (1H, brs).

### Example 396

### 2-[5-(aminomethyl)-6-isobutyl-2-methyl-4-(4-methylphenyl)pyridin-3-yl]-1H-isoindole-1,3(2H)-dione dihydrochloride

1) tert-Butyl {[5-(1,3-dioxo-1,3-dihydro-2H-isoindol-2-yl)-2-isobutyl-6-methyl-4-(4-methylphenyl)pyridin-3-yl]methyl}carbamate (221 mg, yield 94%) was obtained as a white powder from 2-({[5-{[(tert-butoxycarbonyl)amino]methyl}-6-isobutyl-2-methyl-4-(4-methylphenyl)pyridin-3-yl]amino}carbonyl)benzoic acid (260 mg, 0.48 mmol) according to a method similar to the method of Example 3-1).

¹H-NMR (CDCl₃) δ:1.03 (6H, d, J=6.6 Hz), 1.39 (9H, s), 2.02 (3H, s), 2.21-2.31 (1H, m), 2.40 (3H, s), 2.83 (2H, d, J=7.5 Hz), 4.20 (2H, d, J=5.4 Hz), 4.30 (1H, brs), 6.98 (2H, d, J=8.1 Hz), 7.03 (2H, d, J=8.1 Hz), 7.67-7.72 (2H, m), 7.75-7.79 (2H, m).

2) 2-[5-(Aminomethyl)-6-isobutyl-2-methyl-4-(4-methylphenyl)pyridin-3-yl]-1H-isoindole-1,3(2H)-dione dihydrochloride (213 mg, yield 99%) was obtained as a white powder from tert-butyl {[5-(1,3-dioxo-1,3-dihydro-2H-isoindol-2-yl)-2-isobutyl-6-methyl-4-(4-methylphenyl)pyridin-3-yl]methyl}carbamate (221 mg, 0.45 mmol) according to a method similar to the method of Example 2-3).

¹H-NMR (DMSO-d₆) δ:1.01 (6H, d, J=6.3 Hz), 2.19 (3H, s), 2.19-2.32 (1H, m), 2.35 (3H, s), 2.83 (2H, d, J=6.3 Hz), 3.69 (2H, brs), 7.05 (2H, d, J=7.8 Hz), 7.13 (2H, d, J=7.8 Hz), 7.85 (4H, brs), 8.03 (3H, brs).

### Example 397

### methyl 3-[({[5-(aminomethyl)-6-isobutyl-2-methyl-4-(4-methylphenyl)pyridin-3-yl]amino}carbonyl)oxy]benzoate dihydrochloride

1) Methyl 3-[({[5-{[(tert-butoxycarbonyl)amino]methyl}-6-isobutyl-2-methyl-4-(4-methylphenyl)pyridin-3-yl]amino}carbonyl)oxy]benzoate was obtained as an oil from 5-{[(tert-butoxycarbonyl)amino]methyl}-6-isobutyl-2-methyl-4-(4-methylphenyl)nicotinic acid (412 mg, 1.0 mmol) and methyl 3-hydroxybenzoate (304 mg, 2.0 mmol) according to a method similar to the method of Example 95-1).

EIMS(M+1):562

2) Methyl 3-[({[5-(aminomethyl)-6-isobutyl-2-methyl-4-(4-methylphenyl)pyridin-3-yl]amino}carbonyl)oxy]benzoate dihydrochloride (172 mg, yield 32%) was obtained as a white powder from the oil obtained in the aforementioned 1), according to a method similar to the method of Example 2-3).

¹H-NMR (DMSO-d₆)δ:0.98 (6H, d, J=6.6 Hz), 2.14-2.28 (1H, m), 2.44 (3H, s), 2.67 (3H, s), 3.02 (2H, s), 3.85 (2H, s), 3.89 (3H, s), 7.26 (2H, d, J=8.1 Hz), 7.36 (1H, s), 7.39 (2H, d, J=8.1 Hz), 7.53 (1H, t, J=7.8 Hz), 7.80 (1H, d, J=7.8 Hz), 8.37 (3H, brs), 9.75 (1H, brs).

### Example 398

### methyl 4-[({[5-(aminomethyl)-6-isobutyl-2-methyl-4-(4-methylphenyl)pyridin-3-yl]amino}carbonyl)oxy]benzoate dihydrochloride

1) Methyl 4-[({[5-{[(tert-butoxycarbonyl)amino]methyl}-6-isobutyl-2-methyl-4-(4-methylphenyl)pyridin-3-yl]amino}carbonyl)oxy]benzoate was obtained as an oil from 5-{[(tert-butoxycarbonyl)amino]methyl}-6-isobutyl-2-methyl-4-(4-methylphenyl)nicotinic acid (412 mg, 1.0 mmol) and methyl 4-hydroxybenzoate (304 mg, 2.0 mmol) according to a method similar to the method of Example 95-1).

EIMS(M+1) :562

2) Methyl 4-[({[5-(aminomethyl)-6-isobutyl-2-methyl-4-(4-methylphenyl)pyridin-3-yl]amino}carbonyl)oxy]benzoate dihydrochloride (182 mg, yield 34%) was obtained as a white powder from the oil obtained in the aforementioned 1), according to a method similar to the method of Example 2-3).

¹H-NMR (DMSO-d₆)δ:0.98 (6H, d, J=6.6 Hz), 2.14-2.29 (1H, m), 2.43 (3H, s), 2.62 (3H, s), 2.93 (2H, brs), 3.84 (2H, s), 3.85 (3H, s), 7.00 (2H, d, J=8.7 Hz), 7.24 (2H, d, J=8.1 Hz), 7.39 (2H, d, J=8.1 Hz), 7.96 (2H, t, J=8.7 Hz), 8.29 (3H, brs), 9.71 (1H, brs).

### Example 399

### methyl 5-(aminomethyl)-6-isobutyl-2-methyl-4-(4-methylphenyl)pyridin-3-yl]carbamate dihydrochloride

1) Methyl 5-{[(tert-butoxycarbonyl)amino]methyl}-6-isobutyl-2-methyl-4-(4-methylphenyl)pyridin-3-yl]carbamate was obtained as an oil from 5 {[(tert-butoxycarbonyl)amino]methyl}-6-isobutyl-2-methyl-4-(4-methylphenyl)nicotinic acid (412 mg, 1.0 mmol) and methanol (62 mg, 2.0 mmol) according to a method similar to the method of Example 95-1).

### EIMS(M+1):443

2) Methyl 5-(aminomethyl)-6-isobutyl-2-methyl-4-(4-methylphenyl)pyridin-3-yl]carbamate dihydrochloride (330 mg, yield 80%) was obtained as a white powder from the oil obtained in the aforementioned 1), according to a method similar to the method of Example 2-3).

¹H-NMR (DMSO-d₆)δ:0.98 (6H, d, J=6.6 Hz), 2.11-2.18 (1H, m), 2.39 (3H, s), 2.63 (3H, s), 3.11 (2H, s), 3.48 (3H, s), 3.82 (2H, s), 7.18 (2H, d, J=7.8 Hz), 7.33 (2H, d, J=7.8 Hz), 8.44 (3H, brs), 9.03 (1H, brs).

### Example 400

### ethyl {3-[5-(aminomethyl)-6-isobutyl-2-methyl-4-(4-methylphenyl)pyridin-3-yl]-2,4-dioxoimidazolidin-1-yl}acetate dihydrochloride

1) Diethyl 2,2′-[({[5-{[(tert-butoxycarbonyl)amino]methyl}-6-isobutyl-2-methyl-4-(4-methylphenyl)pyridin-3-yl]amino}carbonyl)imino]diacetate was obtained as white crystals (260 mg, yield 43%) from 5-{[(tert-butoxycarbonyl)amino]methyl}-6-isobutyl-2-methyl-4-(4-methylphenyl)nicotinic acid (412 mg, 1.0 mmol) and diethyl 2,2′-iminodiacetate (380 mg, 2.0 mmol) according to a method similar to the method of Example 95-1).

¹H-NMR (CDCl₃)δ:0.96 (6H, d, J=6.6 Hz), 1.24 (6H, t, J=6.9 Hz), 1.38 (9H, s), 2.09-2.24 (1H, m), 2.40 (3H, s), 2.49 (3H, s), 2.75 (2H, d, J=6.9 Hz), 3.87 (4H, s), 4.12 (4H, q, J=6.9 Hz), 4.23 (1H, brs), 6.33 (1H, brs), 7.04 (2H, d, J=7.8 Hz), 7.25 (2H, d, J=7.8 Hz).

2) Ethyl {3-[5-(aminomethyl)-6-isobutyl-2-methyl-4-(4-methylphenyl)pyridin-3-yl]-2,4-dioxoimidazolidin-1-yl}acetate dihydrochloride (240 mg, yield 98%) was obtained as a white powder from diethyl 2,2′-[({[5-{[(tert-butoxycarbonyl)amino]methyl}-6-isobutyl-2-methyl-4-(4-methylphenyl)pyridin-3-yl]amino}carbonyl)imino]diacetate (260 mg, 0.43 mmol) according to a method similar to the method of Example 2-3).

¹H-NMR (DMSO-d₆)δ:0.99 (6H, d, J=6.6 Hz), 1.19 (6H, t, J=7.2 Hz), 2.22-2.35 (1H, m), 2.35 (3H, s), 2.50 (3H, s), 2.86 (2H, d, J=7.2 Hz), 3.74-3.80 (3H, m), 4.02-4.17 (5H, m), 7.04-7.11 (2H, m), 7.21-7.27 (2H, m), 8.25 (3H, brs).

### Example 401

### ethyl 6-({[5-(aminomethyl)-6-isobutyl-2-methyl-4-(4-methylphenyl)pyridin-3-yl]amino}carbonyl)pyridine-2-carboxylate dihydrochloride

Ethyl 6-({[5-(aminomethyl)-6-isobutyl-2-methyl-4-(4-methylphenyl)pyridin-3-yl]amino}carbonyl)pyridine-2-carboxylate dihydrochloride (230 mg, yield 89%) was obtained as a white powder from tert-butyl {[5-amino-2-isobutyl-6-methyl-4-(4-methylphenyl)pyridin-3-yl]methyl}carbamate (192 mg, 0.5 mmol) and ethyl 6-(chlorocarbonyl)pyridine-2-carboxylate (149 mg, 0.75 mmol) according to a method similar to the method of Example 223.

¹H-NMR (DMSO-d₆) δ:1.00 (6H, d, J=6.6 Hz), 1.35 (3H, t, J=7.2 Hz), 2.11-2.28 (1H, m), 2.27 (3H, s), 2.60 (3H, s), 3.05 (2H, brs), 3.84 (2H, brs), 4.37 (2H, q, J=7.2 Hz), 7.22 (1H, d, J=7.8 Hz), 7.26 (2H, d, J=7.8 Hz), 8.21-8.31 (3H, m), 8.38 (3H, brs), 9.90 (1H, brs).

### Example 402

### 6-({[5-(aminomethyl)-6-isobutyl-2-methyl-4-(4-methylphenyl)pyridin-3-yl]amino}carbonyl)pyridine-2-carboxylic acid dihydrochloride

1) 6-({[5-{[(tert-Butoxycarbonyl)amino]methyl}-6-isobutyl-2-methyl-4-(4-methylphenyl)pyridin-3-yl]amino}carbonyl)pyridine-2-carboxylic acid (247 mg, yield 98%) was obtained as a white powder from ethyl 6-({[5-{[(tert-butoxycarbonyl)amino]methyl}-6-isobutyl-2-methyl-4-(4-methylphenyl)pyridin-3-yl]amino}carbonyl)pyridine-2-carboxylate (260 mg, 0.48 mmol) according to a method similar to the method of Example 36-1).

¹H-NMR (CDCl₃) δ:0.98 (6H, d, J=6.6 Hz), 1.38 (9H, s), 2.14-2.26 (1H, m), 2.28 (3H, s), 2.52 (3H, s), 2.84 (2H, brs), 4.15 (2H, s), 4.42 (1H, brs), 7.01 (2H, d, J=7.8 Hz), 7.10 (2H, d, J=7.8 Hz), 7.99 (1H, brs), 8.21-8.31 (2H, m), 9.36 (1H, brs).

2) 6-({[5-(Aminomethyl)-6-isobutyl-2-methyl-4-(4-methylphenyl)pyridin-3-yl]amino}carbonyl)pyridine-2-carboxylic acid dihydrochloride (221 mg, yield 94%) was obtained as a white powder from 6-({[5-{[(tert-butoxycarbonyl)amino]methyl}-6-isobutyl-2-methyl-4-(4-methylphenyl)pyridin-3-yl]amino}carbonyl)pyridine-2-carboxylic acid (247 mg, 0.47 mmol) according to a method similar to the method of Example 2-3).

¹H-NMR (DMSO-d₆) δ:1.00 (6H, d, J=6.6 Hz), 2.11-2.29 (1H, m), 2.25 (3H, s), 2.60 (3H, s), 3.04 (2H, brs), 3.85 (2H, brs), 7.19 (1H, d, J=7.8 Hz), 7.26 (2H, d, J=7.8 Hz), 8.17-8.26 (3H, m), 8.37 (3H, brs), 10.67 (1H, brs).

### Example 403

### N-[5-(aminomethyl)-6-isobutyl-2-methyl-4-(4-methylphenyl)pyridin-3-yl]pyridine-2,6-dicarboxamide dihydrochloride

1) tert-Butyl {[5-({[6-(aminocarbonyl)pyridin-2-yl]carbonyl}amino)-2-isobutyl-6-methyl-4-(4-methylphenyl)pyridin-3-yl]methyl}carbamate (221 mg, yield 94%) was obtained as a white powder from 6-({[5-{[(tert-butoxycarbonyl)amino]methyl}-6-isobutyl-2-methyl-4-(4-methylphenyl)pyridin-3-yl]amino}carbonyl)pyridine-2-carboxylic acid (260 mg, 0.48 mmol) according to a method similar to the method of Example 3-1).

¹H-NMR (CDCl₃) δ:1.00 (6H, d, J=6.6 Hz), 1.39 (9H, s), 2.18-2.29 (1H, m), 2.35 (3H, s), 2.57 (3H, s), 2.79 (2H, d, J=7.5 Hz), 4.15 (2H, brs), 4.29 (1H, brs), 5.53 (1H, brs), 6.75 (1H, brs), 7.07 (2H, d, J=7.8 Hz), 7.19 (2H, d, J=7.8 Hz), 8.02 (1H, t, J=7.8 Hz), 8.29 (1H, dd, J=1.2, 7.8 Hz), 8.31 (1H, dd, J=1.2, 7.8 Hz), 8.74 (1H, s).

2) N-[5-(Aminomethyl)-6-isobutyl-2-methyl-4-(4-methylphenyl)pyridin-3-yl]pyridine-2,6-dicarboxamide dihydrochloride (206 mg, yield 94%) was obtained as a white powder from tert-butyl {[5-({[6-(aminocarbonyl)pyridin-2-yl]carbonyl}amino)-2-isobutyl-6-methyl-4-(4-methylphenyl)pyridin-3-yl]methyl}carbamate (221 mg, 0.45 mmol) according to a method similar to the method of Example 2-3).

¹H-NMR (DMSO-d₆) δ:1.00 (6H, d, J=6.3 Hz), 2.12-2.28 (1H, m), 2.25 (3H, s), 2.63 (3H, s), 3.07 (2H, brs), 3.86 (2H, brs), 7.19 (2H, d, J=7.8 Hz), 7.28 (2H, d, J=7.8 Hz), 7.76 (1H, s), 8.08-8.20 (3H, m), 8.43 (3H, brs), 8.80 (1H, s), 10.77 (1H, brs).

### Example 404

### N-[5-(aminomethyl)-6-isobutyl-2-methyl-4-(4-methylphenyl)pyridin-3-yl]-1-benzyl-4-methoxy-1H-pyrazole-3-carboxamide dihydrochloride

N-[5-(Aminomethyl)-6-isobutyl-2-methyl-4-(4-methylphenyl)pyridin-3-yl]-1-benzyl-4-methoxy-1H-pyrazole-3-carboxamide dihydrochloride (230 mg, yield 81%) was obtained as a white powder from tert-butyl {[5-amino-2-isobutyl-6-methyl-4-(4-methylphenyl)pyridin-3-yl]methyl}carbamate (192 mg, 0.5 mmol) and 1-benzyl-4-methoxy-1H-pyrazole-3-carbonyl chloride (188 mg, 0.75 mmol) according to a method similar to the method of Example 223.

¹H-NMR (DMSO-d₆) δ:0.98 (6H, d, J=6.6 Hz), 2.18-2.26 (1H, m), 2.35 (3H, s), 2.51 (3H, s), 2.91 (2H, brs), 3.67 (3H, s), 3.81 (2H, brs), 5.15 (2H, s), 7.16-7.39 (9H, m), 8.11 (1H, s), 8.21 (3H, brs).

### Example 405

### N-[5-(aminomethyl)-6-isobutyl-2-methyl-4-(4-methylphenyl)pyridin-3-yl]-1,5-dimethyl-1H-pyrazole-3-carboxamide dihydrochloride

N-[5-(Amninomethyl)-6-isobutyl-2-methyl-4-(4-methylphenyl)pyridin-3-yl]-1,5-dimethyl-1H-pyrazole-3-carboxamide dihydrochloride (235 mg, yield 97%) was obtained as a white powder from tert-butyl {[5-amino-2-isobutyl-6-methyl-4-(4-methylphenyl)pyridin-3-yl]methyl}carbamate (192 mg, 0.5 mmol) and 1,5-dimethyl-1H-pyrazole-3-carbonyl chloride (118 mg, 0.75 mmol) according to a method similar to the method of Example 223.

¹H-NMR (DMSO-d₆) δ:0.99 (6H, d, J=6.6 Hz), 2.18-2.25 (1H, m), 2.32 (3H, s), 2.33 (3H, s), 2.53 (2H, brs), 3.73 (3H, s), 3.82 (2H, brs), 6.38 (1H, s), 7.20 (2H, d, J=7.8 Hz), 7.24 (2H, d, J=7.8 Hz), 8.31 (3H, s), 9.58 (1H, brs).

### Example 406

### [5-(aminomethyl)-2-methyl-4-(4-methylphenyl)-6-neopentylpyridin-3-yl]acetic acid dihydrochloride

[5-(Aminomethyl)-2-methyl-4-(4-methylphenyl)-6-neopentylpyridin-3-yl]acetic acid dihydrochloride (0.56 g, yield 94%) was obtained as a white powder from [5-{[(tert-butoxycarbonyl)amino]methyl}-2-methyl-4-(4-methylphenyl)-6-neopentylpyridin-3-yl]acetic acid (0.63 g, 1.43 mmol) according to a method similar to the method of Example 2-3).

¹H-NMR (DMSO-d₆)δ: 1.03 (9H, s), 2.41 (3H, s), 2.73 (3H, brs), 3.19 (2H, brs), 3.35-3.45 (2H, m), 3.75-3.90 (2H, m), 7.16 (2H, d, J=7.4 Hz), 7.38 (2H, d, J=7.4 Hz), 8.16 (3H, brs).

### Example 407

### (5-methyl-2-oxo-1,3-dioxol-4-yl)methyl [5-(aminomethyl)-2-methyl-4-(4-methylphenyl)-6-neopentylpyridin-3-yl]acetate dihydrochloride

1) (5-Methyl-2-oxo-1,3-dioxol-4-yl)methyl [5-{[(tert-butoxycarbonyl)amino]methyl}-2-methyl-4-(4-methylphenyl)-6-neopentylpyridin-3-yl]acetate (0.091 g, yield 28%) was obtained as a white powder from [5-{[(tert-butoxycarbonyl)amino]methyl}-2-methyl-4-(4-methylphenyl)-6-neopentylpyridin-3-yl]acetic acid (0.63 g, 1.43 mmol) according to a method similar to the method of Example 176-1).

¹H-NMR (CDCl₃)δ: 1.02 (9H, s), 1.37 (9H, s), 2.14 (3H, s), 2.40 (3H, s), 2.48 (3H, s), 2.83 (2H, s), 4.09 (2H, d, J=4.9 Hz), 4.10-4.25 (1H, m), 4.76 (2H, s), 6.94 (2H, d, J=7.9 Hz), 7.21 (2H, d, J=7.9 Hz).

2) (5-Methyl-2-oxo-1,3-dioxol-4-yl)methyl [5-(aminomethyl)-2-methyl-4-(4-methylphenyl)-6-neopentylpyridin-3-yl]acetate dihydrochloride (0.085 g, yield 99%) was obtained as a pale-yellow powder from (5-methyl-2-oxo-1,3-dioxol-4-yl)methyl [5-{[(tert-butoxycarbonyl)amino]methyl}-2-methyl-4-(4-methylphenyl)-6-neopentylpyridin-3-yl]acetate (0.090 g, 0.16 mmol) according to a method similar to the method of Example 2-3).

¹H-NMR (DMSO-d₆)δ: 1.01 (9H, s), 2.14 (3H, s), 2.38 (3H, s), 2.71 (3H, brs), 3.13 (2H, brs), 3.52 (2H, brs), 3.73 (2H, brs), 4.92 (2H, s), 7.10 (2H, d, J=7.5 Hz), 7.31 (2H, d, J=7.5 Hz), 8.15 (3H, brs).

### Example 408

### methyl 5-(aminomethyl)-6-isobutyl-2-(methoxymethyl)-4-(4-methylphenyl)nicotinate

1) A mixture of methyl 4-methoxyacetate (5.85 g, 40 mmol), ammonium acetate (15.4 g, 200 mmol), acetic acid (2.3 mL, 40 mmol) and toluene (100 mL) was heated under reflux using a Dean-Stark trap for 10 hrs. The reaction mixture was allowed to cool to room temperature, washed with saturated brine and dried over anhydrous magnesium sulfate. The solvent was evaporated under reduced pressure to give methyl 3-amino-4-methoxybut-2-enoate as a crude product (5.8 g). Methyl 5-cyano-6-isobutyl-2-(methoxymethyl)-4-(4-methylphenyl)-1,4-dihydropyridine-3-carboxylate (7.8 g, yield 55%) was obtained as a pale-yellow powder from the crude product (5.8 g), 5-methyl-3-oxohexanenitrile (5.7 g, purity 87.5%, 40 mmol) and p-tolualdehyde (4.8 g, 40 mmol) according to a method similar to the method of Example 1-2).

¹H-NMR (CDCl₃)δ: 0.97 (6H, dd, J=6.6, 12.8 Hz), 1.80-2.00 (1H, m), 2.20-2.40 (2H, m), 2.31 (3H, s), 3.48 (3H, s), 3.57 (3H, s), 4.56 (1H, s), 4.64 (1H, d, J=16.4 Hz), 4.73 (1H, d, J=16.4 Hz), 7.05-7.15 (5H, m).

2) Methyl 5-cyano-6-isobutyl-2-(methoxymethyl)-4-(4-methylphenyl)nicotinate (7.5 g, yield 99%) was obtained as a white powder from methyl 5-cyano-6-isobutyl-2-(methoxyymethyl)-4-(4-methylphenyl)-1,4-dihydropyridine-3-carboxylate (7.7 g, 22 mmol) according to a method similar to the method of Example 23-3).

¹H-NMR (CDCl₃)δ: 1.00 (6H, d, J=6.6 Hz), 2.20-2.35 (1H, m), 2.41 (3H, s), 2.97 (2H, d, J=7.2 Hz), 3.37 (3H, s), 3.59 (3H, s), 4.71 (2H, s), 7.15-7.35 (4H, m).

3) Methyl 5-(aminomethyl)-6-isobutyl-2-(methoxymethyl)-4-(4-methylphenyl)nicotinate (7.1 g, yield 93%) was obtained as a pale-yellow oil from methyl 5-cyano-6-isobutyl-2-(methoxymethyl)-4-(4-methylphenyl)nicotinate (7.4 g, 21 mmol) according to a method similar to the method of Example 1-4).

¹H-NMR (CDCl₃)δ: 0.97 (6H, d, J=6.8 Hz), 1.22 (2H, brs), 2.15-2.30 (1H, m), 2.39 (3H, s), 2.82 (2H, d, J=7.4 Hz), 3.36 (3H, s), 3.49 (3H, s), 3.67 (2H, s), 4.65 (2H, s), 7.11 (2H, d, J=8.1 Hz), 7.21 (2H, d, J=8.1 Hz).

### Example 409

### {6-methyl-4-(4-methylphenyl)-2-neopentyl-5-[(pyridin-2-ylthio)methyl]pyridin-3-yl}methylamine trihydrochloride

1) tert-Butyl ({6-methyl-4-(4-methylphenyl)-2-neopentyl-5-[(pyridin-2-ylthio)methyl]pyridin-3-yl}methyl)carbamate (480 mg, yield 78%) was obtained as a powder from [5-{[(tert-butoxycarbonyl)amino]methyl}-2-methyl-4-(4-methylphenyl)-6-neopentylpyridin-3-yl]methyl methanesulfonate (600 mg, 1.2 mmol) and 2-mercaptopyridine (145 mg, 1.3 mmol) according to a method similar to the method of Example 33-1).

¹H-NMR (CDCl₃) δ:1.02 (9H, s), 1.37 (9H, s), 2.35 (3H, s), 2.62 (3H, s), 2.83 (2H, s), 4.08 (2H, d, J=4.9 Hz), 4.14 (2H, s), 4.19 (1H, s), 6.91-6.95 (1H, m), 7.03-7.06 (3H, m), 7.17 (2H, d, J=7.91 Hz), 7.39-7.45 (1H, m), 8.31 (1H, d, J=4.1 Hz).

2) {6-Methyl-4-(4-methylphenyl)-2-neopentyl-5-[(pyridin-2-ylthio)methyl]pyridin-3-yl}methylamine trihydrochloride (167 mg, yield 82%) was obtained as a powder from tert-butyl ({6-methyl-4-(4-methylphenyl)-2-neopentyl-5-[(pyridin-2-ylthio)methyl]pyridin-3-yl}methyl)carbamate (200 mg, 0.395 mmol) according to a method similar to the method of Example 2-3).

¹H-NMR (DMSO-d₆) δ:1.03 (9H, s), 2.36 (3H, s), 2.90 (3H, s), 3.28 (2H, s), 3.83 (2H, d, J=4.7 Hz), 4.19 (2H, s), 7.11-7.16 (1H, m), 7.23-7.33 (5H, m), 7.62-7.67 (1H, m), 8.31 (3H, brs), 8.33-8.34 (1H, m).

### Example 410

### {6-methyl-4-(4-methylphenyl)-2-neopentyl-5-[(1H-1,2,4-triazol-3-ylthio)methyl]pyridin-3-yl}methylamine dihydrochloride

1) tert-Butyl ({6-methyl-4-(4-methylphenyl)-2-neopentyl-5-[(4H-1,2,4-triazol-3-ylthio)methyl]pyridin-3-yl}methyl)carbamate (455 mg, yield 2%) was obtained as a powder from [5-{[(tert-butoxycarbonyl)amino]methyl}-2-methyl-4-(4-methylphenyl)-6-neopentylpyridin-3-yl]methyl methanesulfonate (600 mg, 1.2 mmol) and 3-mercapto-1,2,4-triazole (131 mg, 1.3 mmol) according to a method similar to the method of Example 33-1).

¹H-NMR (CDCl₃) δ:1.01 (9H, s), 1.37 (9H, s), 2.37 (3H, s), 2.64 (3H, s), 2.83 (2H, s), 4.08 (2H, d, J=4.9 Hz), 4.12 (2H, s), 4.22 (1H, s), 7.04 (2H, d, J=7.7 Hz), 7.20 (2H, d, J=7.7 Hz), 8.02 (1H, s).

2) {6-Methyl-4-(4-methylphenyl)-2-neopentyl-5-[(1H-1,2,4-triazol-3-ylthio)methyl]pyridin-3-yl}methylamine dihydrochloride (160 mg, yield 85%) was obtained as a powder from tert-butyl ({6-methyl-4-(4-methylphenyl)-2-neopentyl-5-[(4H-1,2,4-triazol-3-ylthio)methyl]pyridin-3-yl}methyl)carbamate (200 mg, 0.403 mmol) according to a method similar to the method of Example 2-3).

¹H-NMR (DMSO-d₆) δ:1.02 (9H, s), 2.39 (3H, s), 2.86 (3H, s), 3.21 (2H, s), 3.81 (2H, d, J=4.1 Hz), 4.08 (2H, s), 7.24 (2H, d, J=8.0 Hz), 7.35 (2H, m, J=8.0 Hz), 8.23 (3H, brs), 8.45 (1H, s).

### Example 411

### [5-[(1H-imidazol-2-ylthio)methyl]-6-methyl-4-(4-methylphenyl)-2-neopentylpyridin-3-yl]methylamine trihydrochloride

1) tert-Butyl {[5-[(1H-imidazol-2-ylthio)methyl]-6-methyl-4-(4-methylphenyl)-2-neopentylpyridin-3-yl]methyl}carbamate (373 mg, yield 75%) was obtained as a powder from [5-{[(tert-butoxycarbonyl)amino]methyl}-2-methyl-4-(4-methylphenyl)-6-neopentylpyridin-3-yl]methyl methanesulfonate (500 mg, 1.0 mmol) and 2-mercaptoimidazole (110 mg, 1.1 mmol) according to a method similar to the method of Example 33-1).

¹H-NMR (CDCl₃) δ:1.01 (9H, s), 1.37 (9H, s), 2.41 (3H, s), 2.55 (3H, s), 2.82 (2H, s), 3.94 (2H, s), 4.06 (2H, d, J=4.9 Hz), 4.20 (1H, s), 6.94 (1H, brs), 7.01 (2H, d, J=7.9 Hz), 7.06 (1H, brs), 7.23 (2H, d, J=7.9 Hz).

2) [5-[(1H-Imidazol-2-ylthio)methyl]-6-methyl-4-(4-methylphenyl)-2-neopentylpyridin-3-yl]methylamine trihydrochloride (160 mg, yield 79%) was obtained as a powder from tert-butyl {[5-[(1H-imidazol-2-ylthio)methyl]-6-methyl-4-(4-methylphenyl)-2-neopentylpyridin-3-yl]methyl}carbamate (200 mg, 0.404 mmol) according to a method similar to the method of Example 2-3).

¹H-NMR (DMSO-d₆) δ:1.01 (9H, s), 2.40 (3H, s), 2.67 (3H, s), 3.07 (2H, brs), 3.74 (2H, brs), 4.17 (2H, s), 7.18 (2H, d, J=7.9 Hz), 7.33 (2H, d, J=7.9 Hz), 7.70 (2H, s), 8.26 (3H, brs).

### Example 412

### {6-methyl-4-(4-methylphenyl)-2-neopentyl-5-[(pyrimidin-2-ylthio)methyl]pyridin-3-yl}methylamine trihydrochloride

1) tert-Butyl ({6-methyl-4-(4-methylphenyl)-2-neopentyl-5-[(pyrimidin-2-ylthio)methyl]pyridin-3-yl}methyl)carbamate (380 mg, yield 77%) was obtained as a powder from [5-{[(tert-butoxycarbonyl)amino]methyl}-2-methyl-4-(4-methylphenyl)-6-neopentylpyridin-3-yl]methyl methanesulfonate (500 mg, 1.0 mmol) and 2-mercaptopyrimidine (123 mg, 1.1 mmol) according to a method similar to the method of Example 33-1).

¹H-NMR (CDCl₃) δ:1.02 (9H, s), 1.37 (9H, s), 2.35 (3H, s), 2.65 (3H, s), 2.83 (2H, s), 4.08 (2H, d, J=4.9 Hz), 4.14 (2H, s), 4.19 (1H, brs), 6.92 (1H, t, J=4.9 Hz), 7.06 (2H, d, J=7.8 Hz), 7.18 (2H, d, J=7.8 Hz), 8.43 (2H, d, J=4.9 Hz).

2) {6-Methyl-4-(4-methylphenyl)-2-neopentyl-5-[(pyrimidin-2-ylthio)methyl]pyridin-3-yl}methylamine trihydrochloride (180 mg, yield 88%) was obtained as a powder from tert-butyl ({6-methyl-4-(4-methylphenyl)-2-neopentyl-5-[(pyrimidin-2-ylthio)methyl]pyridin-3-yl}methyl)carbamate (200 mg, 0.395 mmol) according to a method similar to the method of Example 2-3).

¹H-NMR (DMSO-d₆) δ:1.02 (9H, s), 2.35 (3H, s), 2.85 (3H, s), 3.17 (2H, brs), 3.80 (2H, s), 4.18 (2H, s), 7.21-7.13 (5H, m), 8.22 (3H, brs), 8.57 (2H, d, J=4.9 Hz).

### Example 413

### [5-{[(5-methoxy-1H-benzimidazol-2-yl)thio]methyl}-6-methyl-4-(4-methylphenyl)-2-neopentylpyridin-3-yl]methylamine trihydrochloride

1) tert-Butyl {[5-{[(5-methoxy-1H-benzimidazol-2-yl)thio]methyl}-6-methyl-4-(4-methylphenyl)-2-neopentylpyridin-3-yl]methyl}carbamate (530 mg, yield 92%) was obtained as a powder from [5-{[(tert-butoxycarbonyl)amino]methyl}-2-methyl-4-(4-methylphenyl)-6-neopentylpyridin-3-yl]methyl methanesulfonate (500 mg, 1.0 mmol) and 5-methoxy-2-benzimidazolethiol (198 mg, 1.1 mmol) according to a method similar to the method of Example 33-1).

¹H-NMR (CDCl₃) δ:1.02 (9H, s), 1.37 (9H, s), 2.33 (3H, s), 2.64 (3H, s), 2.83 (2H, s), 3.82 (3H, s), 4.07 (2H, d, J=5.1 Hz), 4.22 (2H, s), 4.25 (1H, s), 6.77-6.84 (2H, m), 7.01 (2H, d, J=7.9 Hz), 7.14-7.16 (3H, m), 7.49 (1H, d, J=8.9 Hz).

2) [5-{[(5-Methoxy-1H-benzimidazol-2-yl)thio]methyl}-6-methyl-4-(4-methylphenyl)-2-neopentylpyridin-3-yl]methylamine trihydrochloride (194 mg, yield 91%) was obtained as a powder from tert-butyl {[5-{[(5-methoxy-1H-benzimidazol-2-yl)thio]methyl}-6-methyl-4-(4-methylphenyl)-2-neopentylpyridin-3-yl]methyl}carbamate (200 mg, 0.365 mmol) according to a method similar to the method of Example 2-3).

¹H-NMR (DMSO-d₆) δ:1.02 (9H, s), 2.30 (3H, s), 2.83 (3H, s), 3.12 (2H, brs), 3.77 (2H, s), 3.81 (3H, s), 4.37 (2H, s), 6.94-7.02 (2H, m), 7.20-7.27 (4H, m), 7.46 (1H, d, J=8.9 Hz), 8.23 (3H, brs).

### Example 414

### methyl 3-{[5-(aminomethyl)-2-methyl-4-(4-methylphenyl)-6-neopentylpyridin-3-yl]methoxy}-1H-pyrazole-5-carboxylate dihydrochloride

1) Methyl 3-{[5-{[(tert-butoxycarbonyl)amino]methyl}-2-methyl-4-(4-methylphenyl)-6-neopentylpyridin-3-yl]methoxy}-1H-pyrazole-5-carboxylate (800 mg, yield 52%) was obtained as a powder from [5-{[(tert-butoxycarbonyl)amino]methyl}-2-methyl-4-(4-methylphenyl)-6-neopentylpyridin-3-yl]methyl methanesulfonate (1.4 g, 2.85 mmol) and methyl 3-hydroxy-1H-pyrazole-5-carboxylate (426 mg, 3.0 mmol) according to a method similar to the method of Example 33-1).

¹H-NMR (CDCl₃) δ:1.02 (9H, s), 1.37 (9H, s), 2.36 (3H, s), 2.62 (3H, s), 2.86 (2H, s), 3.89 (3H, s), 4.13 (2H, d, J=4.5 Hz), 4.20 (1H, brs), 4.84 (2H, s), 6.13 (1H, s), 7.04 (2H, d, J=7.8 Hz), 7.16 (2H, d, J=7.8 Hz), 9.89 (1H, brs).

2) Methyl 3-{[5-(aminomethyl)-2-methyl-4-(4-methylphenyl)-6-neopentylpyridin-3-yl]methoxy}-1H-pyrazole-5-carboxylate dihydrochloride (142 mg, yield 75%) was obtained as a powder from methyl 3-{[5-{[(tert-butoxycarbonyl)amino]methyl}-2-methyl-4-(4-methylphenyl)-6-neopentylpyridin-3-yl]methoxy}-1H-pyrazole-5-carboxylate (200 mg, 0.373 mmol) according to a method similar to the method of Example 2-3).

¹H-NMR (DMSO-d₆) δ:1.03 (9H, s), 2.37 (3H, s), 2.84 (3H, s), 3.23 (2H, brs), 3.81 (3H, s), 3.87 (2H, brs), 4.83 (2H, s), 6.17 (1H, s), 7.25 (2H, d, J=7.9 Hz), 7.33 (1H, d, J=7.9 Hz), 8.29 (3H, brs).

### Example 415

### 3-{[5-(aminomethyl)-2-methyl-4-(4-methylphenyl)-6-neopentylpyridin-3-yl]methoxy}-1H-pyrazole-5-carboxylic acid dihydrochloride

1) 3-{[5-{[(tert-Butoxycarbonyl)amino]methyl}-2-methyl-4-(4-methylphenyl)-6-neopentylpyridin-3-yl]methoxy}-1H-pyrazole-5-carboxylic acid (914 mg, yield 81%) was obtained as a white solid from methyl 3-{[5-{[(tert-butoxycarbonyl)amino]methyl}-2-methyl-4-(4-methylphenyl)-6-neopentylpyridin-3-yl]methoxy}-1H-pyrazole-5-carboxylate (1.16 g, 2.16 mmol) according to a method similar to the method of Example 9-1).

¹H-NMR (DMSO-d₆) δ:1.00 (9H, s), 1.34 (9H, s), 2.32 (3H, s), 2.53 (3H, s), 2.69 (2H, s), 3.87 (2H, d, J=3.2 Hz), 4.73 (2H, s), 6.06 (1H, s), 6.83 (1H, t, J=4.1 Hz), 7.13-7.21 (4H, m), 12.91 (1H, s).

2) 3-{[5-(Aminomethyl)-2-methyl-4-(4-methylphenyl)-6-neopentylpyridin-3-yl]methoxy}-1H-pyrazole-5-carboxylic acid dihydrochloride (180 mg, yield 95%) was obtained as a white powder from 3-{[5-{[(tert-butoxycarbonyl)amino]methyl}-2-methyl-4-(4-methylphenyl)-6-neopentylpyridin-3-yl]methoxy}-1H-pyrazole-5-carboxylic acid (200 mg, 0.383 mmol) according to a method similar to the method of Example 2-3).

¹H-NMR (DMSO-d₆) δ:1.03 (9H, s), 2.37 (3H, s), 2.51 (3H, s), 2.78 (2H, s), 3.85 (2H, s), 4.80 (2H, s), 6.09 (1H, s), 7.23 (2H, d, J=7.9 Hz), 7.32 (2H, d, J=7.9 Hz), 8.16 (3H, brs).

### Example 416 4-(methoxycarbonyl)benzyl 5-(aminomethyl)-2-methyl-4-(4-methylphenyl)-6-neopentylnicotinate dihydrochloride

1) 4-(Methoxycarbonyl)benzyl 5-{[(tert-butoxycarbonyl)amino]methyl}-2-methyl-4-(4-methylphenyl)-6-neopentylnicotinate (7.36 g, yield 70%) was obtained as a white solid from 5-{[(tert-butoxycarbonyl)amino]methyl}-2-methyl-4-(4-methylphenyl)-6-neopentylnicotinic acid (7.8 g, 18.3 mmol) according to a method similar to the method of Example 169-1).

¹H-NMR (CDCl₃) δ:1.01 (9H, s), 1.36 (9H, s), 2.35 (3H, s), 2.53 (3H, s), 2.87 (2H, s), 3.93 (3H, s), 4.17 (2H, s), 4.98 (2H, s), 7.02 (2H, d, J=7.9 Hz), 7.09 (2H, d, J=8.2 Hz), 7.11 (2H, d, J=7.9 Hz), 7.93 (2H, d, J=8.2 Hz).

2) 4-(Methoxycarbonyl)benzyl 5-(aminomethyl)-2-methyl-4-(4-methylphenyl)-6-neopentylnicotinate dihydrochloride (181 mg, yield 95%) was obtained as a white powder from 4-(methoxycarbonyl)benzyl 5-{[(tert-butoxycarbonyl)amino]methyl}-2-methyl-4-(4-methylphenyl)-6-neopentylnicotinate (200 mg, 0.348 mmol) according to a method similar to the method of Example 2-3).

### ¹H-NMR (DMSO-d₆) δ:1.00 (9H, s), 2.33 (3H, s), 2.51 (3H, s), 2.90 (2H, s), 3.83 (2H, s), 3.86 (3H, s), 5.07 (2H, s), 7.12-7.21 (6H, m), 7.87 (2H, d, J=8.3 Hz), 8.13 (3H, brs).

### Example 417

### 4-[({[5-(aminomethyl)-2-methyl-4-(4-methylphenyl)-6-neopentylpyridin-3-yl]carbonyl}oxy)methyl]benzoic acid dihydrochloride

1) 4-[({[5-{[(tert-Butoxycarbonyl)amino]methyl}-2-methyl-4-(4-methylphenyl)-6-neopentylpyridin-3-yl]carbonyl}oxy)methyl]benzoic acid (1.68 g, yield 86%) was obtained as a white solid from 4-(methoxycarbonyl)benzyl 5-{[(tert-butoxycarbonyl)amino]methyl}-2-methyl-4-(4-methylphenyl)-6-neopentylnicotinate (2.0 g, 3.48 mmol) according to a method similar to the method of Example 9-1).

¹H-NMR (CDCl₃) δ:1.01 (9H, s), 1.37 (9H, s), 2.35 (3H, s), 2.55 (3H, s), 2.89 (2H, s), 4.16-4.20 (3H, m), 5.01 (2H, s), 7.02-7.13 (6H, m), 7.99 (2H, d, J=8.3 Hz).

2) 4-[({[5-(Aminomethyl)-2-methyl-4-(4-methylphenyl)-6-neopentylpyridin-3-yl]carbonyl}oxy)methyl]benzoic acid dihydrochloride (150 mg, yield 79%) was obtained as a white powder from 4-[({[5-{[(tert-butoxycarbonyl)amino]methyl}-2-methyl-4-(4-methylphenyl)-6-neopentylpyridin-3-yl]carbonyl}oxy)methyl]benzoic acid (200 mg, 0.357 mmol) according to a method similar to the method of Example 2-3).

¹H-NMR (DMSO-d₆) δ:1.00 (9H, s), 2.34 (3H, s), 2.51 (3H, s), 2.90 (2H, s), 3.84 (2H, d, J=5.7 Hz), 5.06 (2H, s), 7.10-7.18 (6H, m), 7.85 (2H, d, J=8.3 Hz), 8.11 (3H, brs).

### Example 418

### 4-(trifluoromethyl)benzyl [5-(aminomethyl)-6-isobutyl-2-methyl-4-(4-methylphenyl)pyridin-3-yl]acetate dihydrochloride

1) 4-(Trifluoromethyl)benzyl [5-{[(tert-butoxycarbonyl)amino]methyl}-6-isobutyl-2-methyl-4-(4-methylphenyl)pyridin-3-yl]acetate (350 mg, yield 85%) was obtained as a white powder from [5-{[(tert-butoxycarbonyl)amino]methyl}-6-isobutyl-2-methyl-4-(4-methylphenyl)pyridin-3-yl]acetic acid (300 mg, 0.703 mmol) and 1-(bromomethyl)-4-(trifluoromethyl)benzene (250 mg, 1.05 mmol) according to a method similar to the method of Example 169-1).

¹H-NMR (CDCl₃) δ:0.97 (6H, d, J=6.8 Hz), 1.37 (9H, s), 2.11-2.29 (1H, m), 2.37 (3H, s), 2.48 (3H, s), 2.75 (2H, d, J=6.6 Hz), 3.42 (2H, s), 4.03 (2H, d, J=5.1 Hz), 4.20 (1H, brs), 5.09 (2H, s), 6.91 (2H, d, J=7.7 Hz), 7.14 (2H, d, J=7.7 Hz), 7.33 (2H, d, J=8.1 Hz), 7.60 (2H, d, J=8.1 Hz).

2) 4-(Trifluoromethyl)benzyl [5-(aminomethyl)-6-isobutyl-2-methyl-4-(4-methylphenyl)pyridin-3-yl]acetate dihydrochloride (283 mg, yield 66%) was obtained as a white powder from 4-(trifluoromethyl)benzyl [5-{[(tert-butoxycarbonyl)amino]methyl}-6-isobutyl-2-methyl-4-(4-methylphenyl)pyridin-3-yl]acetate (330 mg, 0.564 mmol) according to a method similar to the method of Example 2-3).

¹H-NMR (DMSO-d₆)δ:0.97 (6H, d, J=6.6 Hz), 2.09-2.25 (1H, m), 2.36 (3H, s), 2.77 (3H, s), 3.12 (2H,s), 3.77 (2H, d, J=5.1 Hz), 5.14 (2H, s), 7.09 (2H, d, J=8.1 Hz), 7.24 (2H, d, J=8.1 Hz), 7.47 (2H, d, J=8.1 Hz), 7.76 (2H, d, J=8.1 Hz), 8.35 (3H, brs).

### Example 419

### 4-fluorobenzyl [5-(aminomethyl)-6-isobutyl-2-methyl-4-(4-methylphenyl)pyridin-3-yl]acetate dihydrochloride

1) 4-Fluorobenzyl [5-{[(tert-butoxycarbonyl)amino]methyl}-6-isobutyl-2-methyl-4-(4-methylphenyl)pyridin-3-yl]acetate (325 mg, yield 86%) was obtained as a white powder from [5-{[(tert-butoxycarbonyl)amino]methyl}-6-isobutyl-2-methyl-4-(4-methylphenyl)pyridin-3-yl]acetic acid (300 mg, 0.703 mmol) and 1-(bromomethyl)-4-fluorobenzene (198 mg, 1.05 mmol) according to a method similar to the method of Example 169-1).

¹H-NMR (CDCl₃) δ:0.96 (6H, d, J=6.6 Hz), 1.38 (9H, s), 2.11-2.26 (1H, m), 2.38 (3H, s), 2.46 (3H, s), 2.74 (2H, d, J=7.4 Hz), 3.38 (2H, s), 4.02 (2H, d, J=4.9 Hz), 4.20 (1H, brs), 5.00 (2H, s), 6.90 (2H, d, J=7.9 Hz), 6.88-7.07 (2H, m),7.14 (2H, d, J=7.9 Hz), 7.15-7.25 (2H, m).

2) 4-Fluorobenzyl [5-(aminomethyl)-6-isobutyl-2-methyl-4-(4-methylphenyl)pyridin-3-yl]acetate dihydrochloride (234 mg, yield 82%) was obtained as a white powder from 4-fluorobenzyl [5-{[(tert-butoxycarbonyl)amino]methyl}-6-isobutyl-2-methyl-4-(4-methylphenyl)pyridin-3-yl]acetate (300 mg, 0.561 mmol) according to a method similar to the method of Example 2-3).

¹H-NMR (DMSO-d₆)δ:0.98 (6H, d, J=6.6 Hz), 2.12-2.26 (1H, m), 2.38 (3H, s), 2.84 (3H, s), 3.26 (2H, d, J=6.8 Hz), 3.79 (2H, d, J=4.5 Hz), 5.03 (2H, s), 7.12 (2H, d, J=7.9 Hz), 7.17-7.39 (6H, m), 8.57 (3H, brs).

### Example 420

### {[2-isobutyl-6-methyl-4-(4-methylphenyl)-5-(2-oxo-2-pyrrolidin-1-ylethyl)pyridin-3-yl]methyl}amine dihydrochloride

1) tert-Butyl {[2-isobutyl-6-methyl-4-(4-methylphenyl)-5-(2-oxo-2-pyrrolidin-1-ylethyl)pyridin-3-yl]methyl}carbamate (120 mg, yield 36%) was obtained as a white powder from [5-{[(tert-butoxycarbonyl)amino]methyl}-6-isobutyl-2-methyl-4-(4-methylphenyl)pyridin-3-yl]acetic acid (300 mg, 0.703 mmol) and pyrrolidine (440 mg, 2.11 mmol) according to a method similar to the method of Example 311-1).

¹H-NMR (CDCl₃) δ:0.97 (6H, d, J=6.6 Hz), 1.37 (9H, s), 2.12-2.25 (1H, m), 2.39 (3H, s), 2.55 (3H, s), 2.74 (2H, d, J=7.4 Hz), 2.86-2.97 (4H, m), 3.28 (2H, s), 3.36 (2H, t, J=6.5 Hz), 4.03 (2H, d, J=4.7 Hz), 4.20 (1H, brs), 7.01 (2H, d, J=7.9 Hz), 7.21 (2H, d, J=7.9 Hz).

2) {[2-Isobutyl-6-methyl-4-(4-methylphenyl)-5-(2-oxo-2-pyrrolidin-1-ylethyl)pyridin-3-yl]methyl}amine dihydrochloride (62.4 mg, yield 66%) was obtained as a white powder from tert-butyl {[2-isobutyl-6-methyl-4-(4-methylphenyl)-5-(2-oxo-2-pyrrolidin-1-ylethyl)pyridin-3-yl]methyl}carbamate (100 mg, 0.208 mmol) according to a method similar to the method of Example 2-3).

¹H-NMR (DMSO-d₆)δ:0.99 (6H, d, J=6.6 Hz), 2.11-2.26 (1H, m), 2.40 (3H, s), 2.80 (3H, s), 2.88 (2H, t, J=6.1 Hz), 3.12-3.29 (4H, m), 3.42 (2H, s), 3.81 (2H, s), 7.17 (2H, d, J=7.9 Hz), 7.38 (2H, d, J=7.9 Hz), 8.43 (3H, brs).

### Example 421

### ethyl 1-{[5-(aminomethyl)-6-isobutyl-2-methyl-4-(4-methylphenyl)pyridin-3-yl]acetyl}piperidine-4-carboxylate dihydrochloride

1) Ethyl 1-{[5-{[(tert-butoxycarbonyl)amino]methyl}-6-isobutyl-2-methyl-4-(4-methylphenyl)pyridin-3-yl]acetyl}piperidine-4-carboxylate (330 mg, yield 50%) was obtained as a white powder from [5-{[(tert-butoxycarbonyl)amino]methyl}-6-isobutyl-2-methyl-4-(4-methylphenyl)pyridin-3-yl]acetic acid (500 mg, 1.17 mmol) and ethyl piperidine-4-carboxylate (553 mg, 3.52 mmol) according to a method similar to the method of Example 311-1).

¹H-NMR (CDCl₃) δ:0.97 (6H, d, J=6.6 Hz), 1.27 (3H, t, J=7.2 Hz), 1.37 (9H, s), 1.54 (1H, dd, J=13.2, 9.8 Hz), 1.64-1.75 (1H, m), 1.87 (1H, dd, J=13.2, 2,6 Hz), 2.12-2.27 (1H, m), 2.38 (3H, s), 2.49 (3H, s), 2.74 (2H, d, J=7.2 Hz), 2.81-3.01 (3H, m), 3.30 (2H, s), 3.49-3.60 (1H, m), 4.15 (2H, q, J=7.2 Hz), 4.20 (1H, brs), 6.98 (2H, d, J=8.1 Hz), 7.21 (2H, d, J=8.1 Hz).

2) Ethyl 1-{[5-(aminomethyl)-6-isobutyl-2-methyl-4-(4-methylphenyl)pyridin-3-yl]acetyl}piperidine-4-carboxylate dihydrochloride (8.2 mg, yield 43%) was obtained as a white powder from ethyl 1-{[5-{[(tert-butoxycarbonyl)amino]methyl}-6-isobutyl-2-methyl-4-(4-methylphenyl)pyridin-3-yl]acetyl}piperidine-4-carboxylate (20 mg, 0.0354 mmol) according to a method similar to the method of Example 2-3).

EIMS(M+1):466.

### Example 422

### 1-{[5-(aminomethyl)-6-isobutyl-2-methyl-4-(4-methylphenyl)pyridin-3-yl]acetyl}piperidine-4-carboxylic acid dihydrochloride

1) 1-{[5-{[(tert-Butoxycarbonyl)amino]methyl}-6-isobutyl-2-methyl-4-(4-methylphenyl)pyridin-3-yl]acetyl}piperidine-4-carboxylic acid (240 mg, yield 87%) was obtained as a white powder from ethyl 1-{[5-{[(tert-butoxycarbonyl)amino]methyl}-6-isobutyl-2-methyl-4-(4-methylphenyl)pyridin-3-yl]acetyl}piperidine-4-carboxylate (290 mg, 0.513 mmol) according to a method similar to the method of Example 9-1).

¹H-NMR (CDCl₃) δ:1.01 (6H, d, J=6.4 Hz), 1.37 (9H, s), 1.48-1.62 (1H, m), 1.73 (1H, d, J=11.1 Hz), 1.89 (1H, d, J=10.6 Hz), 2.14-2.29 (1H, m), 2.40 (3H, s), 2.74 (3H, s), 2.77-3.00 (2H, m), 3.06 (2H, d, J=6.0 Hz), 3.42 (2H, s), 3.53 (1H, d, J=12.8 Hz), 4.10 (2H, d, J=5.09 Hz), 4.20 (1H, brs), 4.26 (1H, d, J=12.6 Hz), 4.65 (1H, s), 7.01 (2H, d, J=7.5 Hz), 7.27 (2H, d, J=7.5 Hz).

2) 1-{[5-(Aminomethyl)-6-isobutyl-2-methyl-4-(4-methylphenyl)pyridin-3-yl]acetyl}piperidine-4-carboxylic acid dihydrochloride (220 mg, yield 100%) was obtained as a white powder from 1-{[5-{[(tert-butoxycarbonyl)amino]methyl}-6-isobutyl-2-methyl-4-(4-methylphenyl)pyridin-3-yl]acetyl}piperidine-4-carboxylic acid (230 mg, 0.428 mmol) according to a method similar to the method of Example 2-3).

EIMS(M+1):438

### Example 423

### N-2-adamantyl-2-[5-(aminomethyl)-6-isobutyl-2-methyl-4-(4-methylphenyl)pyridin-3-yl]acetamide dihydrochloride

1) tert-Butyl {[5-[2-(2-adamantylamino)-2-oxoethyl]-2-isobutyl-6-methyl-4-(4-methylphenyl)pyridin-3-yl]methyl}carbamate (50 mg, yield 13%) was obtained as a white powder from [5-{[(tert-butoxycarbonyl)amino]methyl}-6-isobutyl-2-methyl-4-(4-methylphenyl)pyridin-3-yl]acetic acid (300 mg, 0.703 mmol) and 2-adamantanamine (396 mg, 2.11 mmol) according to a method similar to the method of Example 311-1).

¹H-NMR (CDCl₃) δ:0.95 (6H, d, J=6.6 Hz), 1.38 (9H, s), 1.53-1.63 (2H, m), 1.67-1.84 (9H, m), 2.12-2.26 (1H, m), 2.39 (3H, s), 2.57 (3H, s), 2.77 (2H, d, J=7.4 Hz), 3.30 (2H, s), 3.97 (2H, d, J=8.1 Hz), 4.06 (2H, d, J=5.09 Hz), 4.20 (1H, brs), 4.22 (1H, s), 5.45 (1H, d, J=8.3 Hz), 6.96 (2H, d, J=7.9 Hz), 7.22 (2H, d, J=7.9 Hz).

2) N-2-Adamantyl-2-[5-(aminomethyl)-6-isobutyl-2-methyl-4-(4-methylphenyl)pyridin-3-yl]acetamide dihydrochloride (45.1 mg, yield 100%) was obtained as a white powder from tert-butyl {[5-[2-(2-adamantylamino)-2-oxoethyl]-2-isobutyl-6-methyl-4-(4-methylphenyl)pyridin-3-yl]methyl}carbamate (48 mg, 0.0857 mmol) according to a method similar to the method of Example 2-3).

¹H-NMR (DMSO-d₆)δ:0.98 (6H, d, J=6.4 Hz), 1.47 (2H, d, J=12.1 Hz), 1.63-1.94 (12H, m), 2.08-2.26 (1H, m), 2.40 (3H, s), 2.80 (3H, s), 3.22 (2H, d, J=5.84 Hz), 3.44 (2H, s), 3.81 (2H, s), 7.19 (2H, d, J=7.9 Hz), 7.34 (2H, d, J=7.9 Hz), 7.87 (1H, d, J=7.7 Hz), 8.49 (3H, brs).

### Example 424

### 2-[5-(aminomethyl)-6-isobutyl-2-methyl-4-(4-methylphenyl)pyridin-3-yl]-N-(2-thienylmethyl)acetamide dihydrochloride

1) [5-{[(tert-Butoxycarbonyl)amino]methyl}-6-isobutyl-2-methyl-4-(4-methylphenyl)pyridin-3-yl]acetic acid (500 mg, 1.17 mmol) and thiophene-2-methylamine (133 mg, 1.17 mmol) were dissolved in tetrahydrofuran (5 mL) and diethyl cyanophosphonate (286 mg, 1.75 mmol) was added under ice-cooling. The obtained reaction mixture was stirred at room temperature for 16 hrs. The reaction mixture was poured into saturated brine, and the mixture was extracted with ethyl acetate. The extract was washed with saturated aqueous sodium hydrogen carbonate and dried over anhydrous magnesium sulfate. The solvent was evaporated under reduced pressure and the residue was purified by silica gel column chromatography to give tert-butyl [(2-isobutyl-6-methyl-4-(4-methylphenyl)-5-{2-oxo-2-[(2-thienylmethyl)amino]ethyl}pyridin-3-yl)methyl]carbamate (493 mg, yield 81%) as a white powder.

1H-NMR (CDCl₃) δ:0.96 (6H, d, J=6.6 Hz), 1.38 (9H, s), 2.11-2.27 (1H, m), 2.37 (3H, s), 2.56 (3H, s), 2.76 (2H, d, J=7.2 Hz), 3.30 (2H, s), 4.03 (2H, d, J=4.9 Hz), 4.20 (1H, brs), 4.51 (2H, d, J=5.7 Hz), 6.85-7.00 (4H, m), 7.16 (2H, d, J=7.9 Hz), 7.23 (1H, dd, J=5.1, 1,1 Hz).

2) 2-[5-(Aminomethyl)-6-isobutyl-2-methyl-4-(4-methylphenyl)pyridin-3-yl]-N-(2-thienylmethyl)acetamide dihydrochloride (300 mg, yield 66%) was obtained as a white powder from tert-butyl [(2-isobutyl-6-methyl-4-(4-methylphenyl)-5-{2-oxo-2-[(2-thienylmethyl)amino]ethyl}pyridin-3-yl)methyl]carbamate (480 mg, 0.92 mmol) according to a method similar to the method of Example 2-3).

¹H-NMR (DMSO-d₆)δ:0.97 (6H, d, J=6.6 Hz), 2.12-2.33 (1H, m), 2.37 (3H, s), 2.47 (3H, s), 2.59 (2H, s), 3.28 (2H, s), 3.76 (2H, s), 4.37 (2H, d, J=5.8 Hz), 6.89-6.94 (1H, m), 6.97 (1H, dd, J=5.0, 3.5 Hz), 7.43 (1H, dd, J=5.0, 1.2 Hz), 8.04 (3H, brs).

### Example 425

### 2-[5-(aminomethyl)-6-isobutyl-2-methyl-4-(4-methylphenyl)pyridin-3-yl]-N-(pyridin-3-ylmethyl)acetamide trihydrochloride

1) tert-Butyl [(2-isobutyl-6-methyl-4-(4-methylphenyl)-5-{2-oxo-2-[(pyridin-3-ylmethyl)amino]ethyl}pyridin-3-yl)methyl]carbamate (394 mg, yield 65%) was obtained as a white powder from [5-{[(tert-butoxycarbonyl)amino]methyl}-6-isobutyl-2-methyl-4-(4-methylphenyl)pyridin-3-yl]acetic acid (500 mg, 1.17 mmol) and 3-(aminomethyl)pyridine (133 mg, 1.17 mmol) according to a method similar to the method of Example 424-1).

¹H-NMR (CDCl₃) δ:0.97 (6H, d, J=6.6 Hz), 1.37 (9H, s), 2.14-2.29 (1H, m), 2.38 (3H, s), 2.55 (3H, s), 2.75 (2H, d, J=7.2 Hz), 4.02 (2H, d, J=4.9 Hz), 4.20 (1H, brs), 4.35 (2H, d, J=5.8 Hz), 5.47 (1H, s), 6.88 (2H, d, J=7.9 Hz), 7.15 (2H, d, J=7.7 Hz), 7.54 (1H, d, J=7.7 Hz), 8.45 (1H, d, J=1.5 Hz), 8.55 (1H, dd, J=4.7, 1.3 Hz).

2) 2-[5-(Aminomethyl)-6-isobutyl-2-methyl-4-(4-methylphenyl)pyridin-3-yl]-N-(pyridin-3-ylmethyl)acetamide trihydrochloride (380 mg, yield 98%) was obtained as a white powder from tert-butyl [(2-isobutyl-6-methyl-4-(4-methylphenyl)-5-{2-oxo-2-[(pyridin-3-ylmethyl)amino]ethyl}pyridin-3-yl)methyl]carbamate (380 mg, 0.74 mmol) according to a method similar to the method of Example 2-3).

¹H-NMR (DMSO-d₆)δ:0.98 (6H, d, J=6.6 Hz), 2.11-2.24 (1H, m), 2.40 (3H, S), 2.78 (3H, s), 3.20 (2H, d, J=7.4 Hz), 3.43 (2H, s), 4.37 (2H, d, J=5.7 Hz), 7.16 (2H, d, J=8.1 Hz), 7.33 (2H, d, J=8.1 Hz), 8.00 (1H, dd, J=8.0, 5.6 Hz), 8.28 (1H, d, J=8.1 Hz), 8.48 (3H, brs), 8.70-8.77 (1H, m), 8.80-8.85 (1H, m).

### Example 426

### methyl 4-({[5-(aminomethyl)-6-isobutyl-2-methyl-4-(4-methylphenyl)pyridin-3-yl]acetyl}amino)thiophene-3-carboxylate dihydrochloride

1) [5-{[(tert-Butoxycarbonyl)amino]methyl}-6-isobutyl-2-methyl-4-(4-methylphenyl)pyridin-3-yl]acetic acid (500 mg, 1.17 mmol), methyl 4-aminothiophene-3-carboxylate (184 mg, 1.17 mmol) and 0-(7-azabenzotriazol-1-yl)-1,1,3,3-tetramethyluromium hexafluorophosphate (HATU, 1.0 g, 1.75 mmol) were dissolved in N,N-dimethylformamide (10 mL) and the mixture was stirred at room temperature for 24 hrs. The reaction mixture was poured into saturated brine, and the mixture was extracted with ethyl acetate. The extract was washed with saturated aqueous sodium hydrogen carbonate and dried over anhydrous magnesium sulfate. The solvent was evaporated under reduced pressure and the residue was purified by silica gel column chromatography to give methyl 4-({[5-{[(tert-butoxycarbonyl)amino]methyl}-6-isobutyl-2-methyl-4-(4-methylphenyl)pyridin-3-yl]acetyl}amino)thiophene-3-carboxylate (440 mg, yield 66%) as a white powder.

¹H-NMR (CDCl₃) δ:0.98 (6H, d, J=6.4 Hz), 1.40 (9H, s), 2.24-2.33 (1H, m), 2.35 (3H, s), 2.53 (3H, s), 2.77 (2H, d, J=7.2 Hz), 3.52 (2H, s), 3.79 (3H, s), 4.06 (2H, d, J=4.1 Hz), 4.20 (1H, brs), 7.02 (2H, d, J=7.9 Hz), 7.17 (2H, d, J=7.9 Hz), 7.95-7.98 (1H, m), 7.98-8.02 (1H, m).

2) Methyl 4-({[5-(aminomethyl)-6-isobutyl-2-methyl-4-(4-methylphenyl)pyridin-3-yl]acetyl}amino)thiophene-3-carboxylate dihydrochloride (161 mg, yield 65%) was obtained as a white powder from methyl 4-({[5-{[(tert-butoxycarbonyl)amino]methyl}-6-isobutyl-2-methyl-4-(4-methylphenyl)pyridin-3-yl]acetyl}amino)thiophene-3-carboxylate (262 mg, 0.46 mmol) according to a method similar to the method of Example 2-3).

¹H-NMR (DMSO-d₆)δ:0.98 (6H, d, J=6.6 Hz), 2.11-2.27 (1H, m), 2.35 (3H, s), 2.48 (3H, s), 2.80 (2H, s), 3.14 (2H, s), 3.76-3.86 (5H, m), 7.17 (2H, d, J=7.9 Hz), 7.32 (2H, d, J=7.9 Hz), 7.80 (1H, d, J=3.2 Hz), 8.26-8.45 (3H brs), 9.69 (s, 1H).

### Example 427

### 4-({[5-(aminomethyl)-6-isobutyl-2-methyl-4-(4-methylphenyl)pyridin-3-yl]acetyl}amino)thiophene-3-carboxylic acid dihydrochloride

1) 4-({[5-{[(tert-Butoxycarbonyl)amino]methyl}-6-isobutyl-2-methyl-4-(4-methylphenyl)pyridin-3-yl]acetyl}amino)thiophene-3-carboxylic acid (183 mg, yield 67%) was obtained as a white powder from methyl 4-({[5-{[(tert-butoxycarbonyl)amino]methyl}-6-isobutyl-2-methyl-4-(4-methylphenyl)pyridin-3-yl]acetyl}amino)thiophene-3-carboxylate (280 mg, 0.495 mmol) according to a method similar to the method of Example 9-1).

¹H-NMR (CDCl₃) δ:0.98 (6H, d, J=6.6 Hz), 1.40 (9H, s), 2.11-2.24 (1H, m), 2.36 (3H, s), 2.52 (3H, s), 2.78 (2H, s), 3.49 (2H, s), 4.03 (2H, s), 4.20 (1H, brs), 6.98-7.25 (4H, m), 7.85-8.05 (2H, m).

2) 4-({[5-(Aminomethyl)-6-isobutyl-2-methyl-4-(4-methylphenyl)pyridin-3-yl]acetyl}amino)thiophene-3-carboxylic acid dihydrochloride (143 mg, yield 64%) was obtained as a white powder from 4-({[5-{[(tert-butoxycarbonyl)amino]methyl}-6-isobutyl-2-methyl-4-(4-methylphenyl)pyridin-3-yl]acetyl}amino)thiophene-3-carboxylic acid (170 mg, 0.428 mmol) according to a method similar to the method of Example 2-3).

¹H-NMR (DMSO-d₆)δ:0.99 (6H, d, J=6.6 Hz), 2.11-2.27 (1H, m), 2.35 (3H, s), 2.50 (3H, s), 2.79 (2H, s), 3.14 (2H, s), 3.81 (2H, s), 7.17 (2H, d, J=8.1 Hz), 7.30 (2H, d, J=8.1 Hz), 7.79 (1H, d, J=3.6 Hz), 8.29 (1H, d, J=3.6 Hz), 8.33-8.44 (3H, s), 9.89 (1H, s).

### Example 428

### methyl 4-({[5-(aminomethyl)-6-isobutyl-2-methyl-4-(4-methylphenyl)pyridin-3-yl]acetyl}amino)benzoate dihydrochloride

1) Methyl 4-({[5-{[(tert-butoxycarbonyl)amino]methyl}-6-isobutyl-2-methyl-4-(4-methylphenyl)pyridin-3-yl]acetyl}amino)benzoate (442 mg, yield 67%) was obtained as a white powder from [5-{[(tert-butoxycarbonyl)amino]methyl}-6-isobutyl-2-methyl-4-(4-methylphenyl)pyridin-3-yl]acetic acid (500 mg, 1.17 mmol) and methyl 4-aminobenzoate (177 mg, 1.17 mmol) according to a method similar to the method of Example 426-1).

¹H-NMR (CDCl₃) δ:0.98 (6H, d, J=6.6 Hz), 1.38 (9H, s), 2.15-2.28 (1H, m), 2.63 (3H, s), 2.77 (2H, d, J=7.4 Hz), 3.47 (2H, s), 3.89 (3H, s), 4.06 (2H, d, J=5.1 Hz), 4.20 (1H, brs), 7.01 (2H, d, J=7.9 Hz), 7.23 (2H, d, J=7.9 Hz), 7.42 (2H, d, J=8.7 Hz), 7.97 (2H, d, J=8.7 Hz).

2) Methyl 4-({[5-(aminomethyl)-6-isobutyl-2-methyl-4-(4-methylphenyl)pyridin-3-yl]acetyl}amino)benzoate dihydrochloride (142 mg, yield 97%) was obtained as a white powder from methyl 4-({[5-{[(tert-butoxycarbonyl)amino]methyl}-6-isobutyl-2-methyl-4-(4-methylphenyl)pyridin-3-yl]acetyl}amino)benzoate (154 mg, 0.275 mmol) according to a method similar to the method of Example 2-3).

¹H-NMR (DMSO-d₆)δ:0.99 (6H, d, J=6.6 Hz), 2.10-2.30 (1H, m), 2.36 (3H, s), 2.49 (3H, s), 2.71 (2H, s), 3.01 (2H, s), 3.77 (2H, s), 3.82 (3H, s), 7.17 (2H, d, J=8.1 Hz), 7.32 (2H, d, J=8.1 Hz), 7.62 (2H, d, J=8.9 Hz), 7.90 (2H, d, J=8.9 Hz), 8.15 (3H, brs).

### Example 429

### 4-({[5-(aminomethyl)-6-isobutyl-2-methyl-4-(4-methylphenyl)pyridin-3-yl]acetyl}amino)benzoic acid dihydrochloride

1) 4-({[5-{[(tert-Butoxycarbonyl)amino]methyl}-6-isobutyl-2-methyl-4-(4-methylphenyl)pyridin-3-yl]acetyl}amino)benzoic acid (275 mg, yield 100%) was obtained as a white powder from methyl 4-({[5-{[(tert-butoxycarbonyl)amino]methyl}-6-isobutyl-2-methyl-4-(4-methylphenyl)pyridin-3-yl]acetyl}amino)benzoate (280 mg, 0.500 mmol) according to a method similar to the method of Example 9-1).

¹H-NMR (CDCl₃) δ:0.99 (6H, d, J=6.2 Hz), 1.37 (9H, s), 2.12-2.27 (1H, m), 2.35 (3H, s), 2.87 (3H, s), 3.19 (2H, s), 3.87 (2H, s), 4.15 (2H, d, J=6.2 Hz), 4.20 (1H, brs), 7.10 (2H, d, J=8.1 Hz), 7.25 (2H, d, J=8.1 Hz), 7.68 (2H, d, J=8.5 Hz), 8.68 (2H, d, J=8.5 Hz).

2) 4-({[5-(Aminomethyl)-6-isobutyl-2-methyl-4-(4-methylphenyl)pyridin-3-yl]acetyl}amino)benzoic acid dihydrochloride (235 mg, yield 92%) was obtained as a white powder from 4-({[5-{[(tert-butoxycarbonyl)amino]methyl}-6-isobutyl-2-methyl-4-(4-methylphenyl)pyridin-3-yl]acetyl}amino)benzoic acid (270 mg, 0.495 mmol) according to a method similar to the method of Example 2-3).

¹H-NMR (DMSO-d₆)δ:1.00 (6H, d, J=6.6 Hz), 2.12-2.28 (1H, m), 2.37 (3H, s), 2.50 (3H, s), 2.80 (2H, s), 3.15 (2H, s), 3.82 (2H, s), 7.20 (2H, d, J=8.1 Hz), 7.34 (2H, d, J=8.1 Hz), 7.60 (2H, d, J=8.9 Hz), 7.87 (2H, d, J=8.9 Hz), 8.35 (3H, brs).

### Example 430

### ethyl 2-[({[5-(aminomethyl)-6-isobutyl-2-methyl-4-(4-methylphenyl)pyridin-3-yl]acetyl}amino)methyl]-1,3-thiazole-4-carboxylate dihydrochloride

1) Ethyl 2-({[(benzyloxy)carbonyl]amino}methyl)-1,3-thiazole-4-carboxylate (3.5 g, 10.9 mmol) was dissolved in 30% hydrogen bromide acetic acid solution (50 mL), and the solution was stirred at room temperature for 2 hrs. White precipitate was collected by filtration and dissolved in saturated aqueous sodium hydrogen carbonate. The obtained solution was concentrated under reduced pressure, and the residue was dissolved in ethyl acetate. The insoluble material was filtered off and the filtrate was concentrated under reduced pressure to give ethyl 2-(aminomethyl)-1,3-thiazole-4-carboxylate (793 mg, yield 40%) as an oil. Ethyl 2-[({[5-{[(tert-butoxycarbonyl)amino]methyl}-6-isobutyl-2-methyl-4-(4-methylphenyl)pyridin-3-yl]acetyl}amino)methyl]-1,3-thiazole-4-carboxylate (649 mg, yield 100%) was obtained as a white powder from the oil (793 mg) and [5-{[(tert-butoxycarbonyl)amino]methyl}-6-isobutyl-2-methyl-4-(4-methylphenyl)pyridin-3-yl]acetic acid (454 mg, 1.07 mmol) according to a method similar to the method of Example 424-1).

¹H-NMR (CDCl₃) δ:0.97 (6H, d, J=6.6 Hz), 1.35-1.47 (12H, m), 2.13-2.28 (1H, m), 2.36 (3H, s), 2.53 (3H, s), 2.75 (2H, d, J=7.2 Hz), 3.34 (2H, s), 4.03 (2H, d, J=5.3 Hz), 4.20 (1H, brs), 4.43 (2H, q, J=7.2 Hz), 4.66 (2H, d, J=6.0 Hz), 6.93 (2H, d, J=7.9 Hz), 7.14 (2H, d, J=7.9 Hz), 8.14 (1H, s).

2) Ethyl 2-[({[5-(aminomethyl)-6-isobutyl-2-methyl-4-(4-methylphenyl)pyridin-3-yl]acetyl}amino)methyl]-1,3-thiazole-4-carboxylate dihydrochloride (138 mg, yield 81%) was obtained as a white powder from ethyl 2-[({[5-{[(tert butoxycarbonyl)amino]methyl}-6-isobutyl-2-methyl-4-(4-methylphenyl)pyridin-3-yl]acetyl}amino)methyl]-1,3-thiazole-4-carboxylate (178 mg, 0.299 mmol) according to a method similar to the method of Example 2-3).

¹H-NMR (DMSO-d₆)δ:0.98 (6H, d, J=6.4 Hz), 1.31 (3H, t, J=7.2 Hz), 2.10-2.23 (1H, m), 2.38 (3H, s), 2.49 (3H, s), 2.77 (2H, s), 3.14 (2H, s), 3.41 (2H, s), 3.80 (2H, s), 4.31 (2H, q, J=7.2 Hz), 4.51 (2H, d, J=5.8 Hz), 7.17 (2H, d, J=8.1 Hz), 7.32 (2H, d, J=8.1 Hz), 8.36 (3H, brs), 8.91 (1H, s).

### Example 431

### 2-[({[5-(aminomethyl)-6-isobutyl-2-methyl-4-(4-methylphenyl)pyridin-3-yl]acetyl}amino)methyl]-1,3-thiazole-4-carboxylic acid dihydrochloride

1) 2-[({[5-{[(tert-Butoxycarbonyl)amino]methyl}-6-isobutyl-2-methyl-4-(4-methylphenyl)pyridin-3-yl]acetyl}amino)methyl]-1,3-thiazole-4-carboxylic acid (438 mg, yield 100%) was obtained as a white powder from ethyl 2-[({[5-{[(tert-butoxycarbonyl)amino]methyl}-6-isobutyl-2-methyl-4-(4-methylphenyl)pyridin-3-yl]acetyl}amino)methyl]-1,3-thiazole-4-carboxylate (460 mg, 0.773 mmol) according to a method similar to the method of Example 9-1).

¹H-NMR (CDCl₃) δ: 0.93 (6H, d, J=6.6 Hz), 1.34 (9H, s), 2.09-2.26 (1H, m), 2.34 (3H, s), 2.40 (2H, s), 2.48 (3H, s), 3.24 (2H, s), 3.80 (2H, s), 4.20 (1H, brs), 4.48 (2H, d, J=5.8 Hz), 7.09 (2H, d, J=7.0 Hz), 7.19 (2H, d, J=7.0 Hz), 8.39 (1H, s).

2) 2-[({[5-(Aminomethyl)-6-isobutyl-2-methyl-4-(4-methylphenyl)pyridin-3-yl]acetyl}amino)methyl]-1,3-thiazole-4-carboxylic acid dihydrochloride (235 mg, yield 91%) was obtained as a white powder from 2-[({[5-{[(tert-butoxycarbonyl)amino]methyl}-6-isobutyl-2-methyl-4-(4-methylphenyl)pyridin-3-yl]acetyl}amino)methyl]-1,3-thiazole-4-carboxylic acid (270 mg, 0.495 mmol) according to a method similar to the method of Example 2-3).

¹H-NMR (DMSO-d₆)δ: 1.00 (6H, d, J=6.6 Hz), 2.12-2.28 (1H, m), 2.37 (3H, s), 2.50 (3H, s), 2.80 (2H, s), 3.15 (2H, s), 3.82 (2H, s), 7.20 (2H, d, J=8.1 Hz), 7.34 (2H, d, J=8.1 Hz), 7.60 (2H, d, J=8.9 Hz), 7.87 (2H, d, J=8.9 Hz), 8.35 (3H, brs).

### Example 432

### methyl 1-{[5-(aminomethyl)-6-isobutyl-2-methyl-4-(4-methylphenyl)pyridin-3-yl]acetyl}prolinate dihydrochloride

1) Methyl 1-{[5-{[(tert-butoxycarbonyl)amino]methyl}-6-isobutyl-2-methyl-4-(4-methylphenyl)pyridin-3-yl]acetyl}prolinate (456 mg, yield 72%) was obtained as a white powder from [5-{[(tert-butoxycarbonyl)amino]methyl}-6-isobutyl-2-methyl-4-(4-methylphenyl)pyridin-3-yl]acetic acid (500 mg, 1.17 mmol) and methyl proline monohydrochloride (194 mg, 1.17 mmol) according to a method similar to the method of Example 426-1).

¹H-NMR (CDCl₃) δ:0.98 (6H, d, J=6.6 Hz), 1.37 (9H, s), 1.84-2.00 (3H, m), 2.05 (3H, s), 2.08-2.24 (2H, m), 2.75 (3H, s), 3.15-3.26 (2H, m), 3.48 (2H, s), 3.71 (3H, s), 4.11-4.21 (3H, m), 4.31-4.55 (2H, m), 7.02-7.15 (2H, m), 7.28-7.41 (2H, m).

2) Methyl 1-{[5-(aminomethyl)-6-isobutyl-2-methyl-4-(4-methylphenyl)pyridin-3-yl]acetyl}prolinate dihydrochloride (277.5 mg, yield 64%) was obtained as a white powder from methyl 1-{[5-{[(tert-butoxycarbonyl)amino]methyl}-6-isobutyl-2-methyl-4-(4-methylphenyl)pyridin-3-yl]acetyl}prolinate (456 mg, 0.848 mmol) according to a method similar to the method of Example 2-3).

¹H-NMR (DMSO-d₆)δ:0.97 (6H, d, J=6.4 Hz), 1.76-1.91 (3H, m), 2.04-2.24 (2H, m), 2.40 (3H, s), 2.65 (3H, s), 2.96 (2H, s), 3.17 (2H, t, J=6.7 Hz), 3.42 (2H, s), 3.61 (3H, s), 3.77 (2H, s), 4.19-4.32 (2H, m), 7.15 (2H, d, J=7.4 Hz), 7.37 (2H, d, J=7.4 Hz), 8.10 (3H, s).

### Example 433

### N-[5-(aminomethyl)-6-isobutyl-2-methyl-4-(4-methylphenyl)pyridin-3-yl]-3-(5-oxo-4,5-dihydro-1,2,4-oxadiazol-3-yl)benzamide dihydrochloride

To a solution of tert-butyl {[5-amino-2-isobutyl-6-methyl-4-(4-methylphenyl)pyridin-3-yl]methyl}carbamate (383 mg, 1.0 mmol) in tetrahydrofuran (5 mL) was added 3-cyanobenzoyl chloride (245 mg, 1.5 mmol) and triethylamine (280 μL, 2.0 mmol) was added. The mixture was stirred for 18 hrs. Saturated aqueous sodium hydrogen carbonate solution (5 mL) was added to the reaction mixture and the mixture was extracted with ethyl acetate. The organic layer was washed with saturated brine and dried over anhydrous magnesium sulfate. The solvent was evaporated under reduced pressure and the obtained residue was purified by silica gel column chromatography to give an oil. To a solution of the obtained oil in ethanol (5 mL) was added hydoxylamine hydrochloride (192 mg, 3.0 mmol) and sodium carbonate (420 mg, 4.0 mmol) and the mixture was stirred at 80° C. for 15 hrs. Distilled water (10 mL) was added to the reaction mixture and the mixture was extracted with ethyl acetate. The organic layer was washed with saturated brine and dried over anhydrous magnesium sulfate. The solvent was evaporated under reduced pressure to give an oil. To a solution of the obtained oil in tetrahydrofuran (3 mL) was added N,N′-carbonyldiimidazole (324 mg, 2.0 mmol) and the mixture was stirred at 65° C. for 2 hrs. Saturated aqueous sodium carbonate solution (5 mL) was added to the reaction mixture and the mixture was extracted with ethyl acetate. The organic layer was washed with saturated brine and dried over anhydrous magnesium sulfate. The solvent was evaporated under reduced pressure and the obtained residue was purified by silica gel column chromatography to give an oil.

To a solution of the obtained oil in ethyl acetate (2 mL) was added 4N hydrogen chloride ethyl acetate solution (2 mL) and the mixture was stirred at room temperature for 3 hrs. The solvent was evaporated under reduced pressure and the obtained residue was crystallized from hexane to give N-[5-(aminomethyl)-6-isobutyl-2-methyl-4-(4-methylphenyl)pyridin-3-yl]-3-(5-oxo-4,5-dihydro-1,2,4-oxadiazol-3-yl)benzamide dihydrochloride (115 mg, yield 21%) as a white powder.

¹H-NMR (DOSO-d₆)δ:0.99 (6H, d, J=6.6 Hz), 2.21-2.29 (1H, m), 2.29 (3H, s), 2.50 (3H, s), 2.96 (2H, s), 3.82 (2H, s), 7.21 (4H, s), 7.62 (1H, t, J=7.5 Hz), 7.79 (1H, d, J=7.5 Hz), 7.93 (1H, d, J=7.5 Hz), 8.25 (3H, brs), 10.13 (1H, brs), 13.12 (1H, brs).

### Experimental Example 1

Determination of Dipeptidyl Peptidase IV Inhibitory Activity in Rat Plasma

The reaction was carried out according to the method of Raymond et al. (Diabetes, vol. 47, pp. 1253-1258, 1998) using a 96 well flat-bottomed plate at 30° C. An N,N-dimethylformamide solution (1 μL) containing the test compound was added to a mixture of water (69 μL), 1 M Tris-hydrochloride buffer (10 μL, pH 7.5) and 1 mM aqueous Gly-Pro-p-NA solution (100 μL) to prepare a mixed solution. Plasma (20 μL) prepared from blood of SD rat by a conventional method was added to the above-mentioned mixed solution and the enzyme reaction was started at 30° C. The absorbance after 0 hr. and 1 hr. was measured using a microplate reader at a wavelength of 405 nm and an increase (ΔODs) was determined. At the same time, an increase (ΔODc) in absorbance of the reaction mixture without the test compound, and an increase (ΔODb) in absorbance of the reaction mixture without the test compound and the enzyme were determined and percent inhibition of dipeptidyl peptidase IV enzyme activity was calculated from the following formula:
{1-[(ΔODs−ΔODb)/(ΔODc−ΔODb)]}×100

The dipeptidyl peptidase IV inhibitory activity of the test compound group is expressed in IC₅₀ value (nM) and shown Table 5.

TABLE 5

| Test compound (Example No.) | IC₅₀ value (nM) |
|---|---|
| 1 | 520 |

As shown above, the compound of the present invention has a superior dipeptidyl peptidase IV inhibitory activity, and is useful as an agent for the prophylaxis or treatment of diabetes and the like.

### Experimental Example 2

Determination of Dipeptidyl Peptidase IV Inhibitory Activity in Rat Plasma

In the same manner as in Experimental Example 1, the dipeptidyl peptidase IV inhibitory activity of the test compound was determined. The results are shown in Table 6.

TABLE 6

| Test compound (Example No.) | IC₅₀ value (nM) |
|---|---|
| 13 | 25 |
| 22 | 12 |
| 26 | 5.1 |
| 40 | 56 |
| 170 | 100 |
| 210 | 12 |
| 267 | 7.4 |
| 305 | 3.5 |
| 312 | 20 |
| 336 | 19 |
| 350 | 15 |
| 421 | 16 |
| 422 | 7.3 |

As mentioned above, the compound of the present invention has a superior dipeptidyl peptidase IV inhibitory activity, and therefore, is useful as an agent for the prophylaxis or treatment of diabetes and the like.

### Formulation Example 1

### Production of Capsules

| | |
|---|---|
| 1) compound of Example 1 | 30 mg |
| 2) fine cellulose powder | 10 mg |
| 3) lactose | 19 mg |
| 4) magnesium stearate | 1 mg |
| total | 60 mg |

1), 2), 3) and 4) are mixed and filled in gelatin capsules.

### Formulation Example 2

### Production of Tablets

| | |
|---|---|
| 1) compound of Example 1 | 30 g |
| 2) lactose | 50 g |
| 3) corn starch | 15 g |
| 4) carboxymethylcellulose calcium | 44 g |
| 5) magnesium stearate | 1 g |
| total of 1000 tablets | 140 g |

The entire amounts of 1), 2) and 3), and 30 g of 4) are kneaded with water, dried in vacuo and granulated. The granules are mixed with 14 g of 4) and 1 g of 5) and the mixture is compressed with a tableting machine, whereby 1000 tablets containing 30 mg of compound of Example 1 per tablet are obtained.

### INDUSTRIAL APPLICABILITY

The compound of the present invention shows a superior peptidase-inhibitory activity and is useful as an agent for the prophylaxis or treatment of diabetes and the like.

This application is based on patent application Nos. 373776/2003, 30491/2004 and 165977/2004 filed in Japan, the contents of which are hereby incorporated by reference.

1. A compound represented by the formula 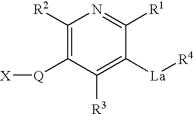
wherein
R¹ and R² are the same or different and each is an optionally substituted hydrocarbon group or an optionally substituted hydroxy group;
R³ is an optionally substituted aromatic group;
R⁴ is an optionally substituted amino group;
L is a divalent chain hydrocarbon group;
Q is a bond or a divalent chain hydrocarbon group; and
X is a hydrogen atom, a cyano group, a nitro group, an acyl group, a substituted hydroxy group, an optionally substituted thiol group, an optionally substituted amino group or an optionally substituted cyclic group; provided that
when X is an ethoxycarbonyl group, then Q is a divalent chain hydrocarbon group, and that the compound is not 2,6-diisopropyl-3-methylaminomethyl-4-(4-fluorophenyl)-5-pentylpyridine;
2,6-diisopropyl-3-aminomethyl-4-(4-fluorophenyl)-5-pentylpyridine;
2,6-diisopropyl-3-(dimethylamino)methyl-4-(4-fluorophenyl)-5-pentylpyridine;
2,6-diisopropyl-3-(ethylamino)methyl-4-(4-fluorophenyl)-5-pentylpyridine; and
3-(tert-butyldimethylsilyloxymethyl)-2,6-diisopropyl-4-(4-fluorophenyl)-5-(indolyl-5-aminomethyl)pyridine, or a salt thereof.

2. The compound of claim 1, wherein R¹ and R² are the same or different and each is an optionally substituted hydrocarbon group, and X is a cyano group, a nitro group, an acyl group, a substituted hydroxy group, an optionally substituted thiol group or an optionally substituted cyclic group.

3. The compound of claim 1, wherein the acyl group for X is a carboxyl group.

4. The compound of claim 1, wherein R¹ and R² are the same or different and each is a C₁₋₁₀ alkyl group optionally substituted by 1 to 3 substituent(s) selected from a C₃₋₁₀ cycloalkyl group, a C₁₋₆ alkoxy-carbonyl group and a C₁₋₆ alkoxy group.

5. The compound of claim 1, wherein R³ is a C₆₋₁₄ aryl group optionally substituted by 1 to 3 substituent(s) selected from a C₁₋₆ alkyl group optionally substituted by 1 to 3 halogen atom(s) and a halogen atom.

6. The compound of claim 1, wherein R⁴ is an amino group.

7. The compound of claim 1, wherein L is a C₁₋₁₀ alkylene group.

8. The compound of claim 1, wherein Q is a bond.

9. The compound of claim 1, wherein X is an acyl group, a substituted hydroxy group, an optionally substituted thiol group or an optionally substituted amino group.

10. The compound of claim 1, wherein X is a carboxyl group.

11. The compound of claim 1, which is 5-(aminomethyl)-2-methyl-4-(4-methylphenyl)-6-neopentylnicotinic acid;
5-(aminomethyl)-6-isobutyl-2-methyl-4-(4-methylphenyl)nicotinic acid;
methyl 3-{[5-(aminomethyl)-6-isobutyl-2-methyl-4-(4-methylphenyl)pyridin-3-yl]methoxy}-1-methyl-1H-pyrazole-4-carboxylate;
{[2-isobutyl-6-methyl-4-(4-methylphenyl)-5-(2-morpholin-4-yl-2-oxoethyl)pyridin-3-yl]methyl}amine;
methyl 3-({[5-(aminomethyl)-6-isobutyl-2-methyl-4-(4-methylphenyl)pyridin-3-yl]acetyl}amino)benzoate;
N-[5-(aminomethyl)-6-isobutyl-2-methyl-4-(4-methylphenyl)pyridin-3-yl]isoxazole-4-carboxamide, or a salt thereof.

12. A prodrug of a compound of claim 1 or a salt thereof.

13. A pharmaceutical agent comprising a compound of claim 1 or a salt thereof or a prodrug thereof.

14. The pharmaceutical agent of claim 13, which is an agent for the prophylaxis or treatment of diabetes, diabetic complications, impaired glucose tolerance or obesity.

15. A peptidase inhibitor comprising a compound of claim 1 or a salt thereof or a prodrug thereof.

16. The inhibitor of claim 15, wherein the peptidase is dipeptidyl dipeptidase-IV.

17.-18. (canceled)

19. A method for the prophylaxis or treatment of diabetes, diabetic complications, impaired glucose tolerance or obesity in a mammal, which comprises administering a compound of claim 1 or a salt thereof or a prodrug thereof to the mammal.

20. A method of inhibiting peptidase in a mammal, which comprises administering a compound of claim 1 or a salt thereof or a prodrug thereof to the mammal.

21. A production method of a compound represented by the formula 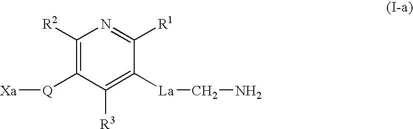
wherein
R¹, R², R³ and Q
are as defined in claim 1;
La is a bond or a divalent chain hydrocarbon group; and
Xa is a hydrogen atom, a nitro group, an acyl group, a substituted hydroxy group, an optionally substituted thiol group, an optionally substituted amino group or an optionally substituted cyclic group;
or a salt thereof, which comprises subjecting a compound represented by the formula 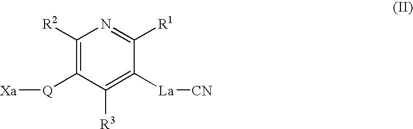
wherein each symbol is as defined above, or a salt thereof to a reduction reaction.
